SEQUENCE LISTING (135 sequences)

SEQ ID NO: 1 - DNA, 822 bp, Homo sapiens
  misc_feature (327)..(327): unknown nucleotide
  misc_feature (396)..(396): unknown nucleotide
gaggcgccttgggaccgcgtgggagccgcagccgaaccgagtagggaccgggaccgcgcggcgccgccgtccccggccgggcccggcccccgcgagccgagcgcgcgcccccgtcgcccacccgggcgcggctggatgcggcggggtccccgcggcggcgacccccggccccgagcgcccggagcgcccagaggcggcgtgcggggcccggggacgccgcgccctstbgtgcgccgaggcgcgccccgagacagccgggggcccgcgccgcagccgccgcccgcgctgagccccggcccggcccgcggcccgcgcccggcggcagcntgagccaggccgagctgtccacctgctccgcgccgcagacgcagcgcatcttccaggaggctgtgcgcnagggcaacacgcaggagctgcagtygctgctgcagaacatgaccaactgcgagttcaacgtgaactcgttcgggcccgagggccagacggcgctgcaccagtcggtcatcgtcggcaacctggtgctcgtgaagctgctggtcaagttcggcgccgacatccgcctggccaaccgcgacggctggagcgcgctgcamatcgccgcgttcggtggccaccaggacatcgtgctctatctcatcaccaaggcgaagtacgcggccagcgcsggtgtatgcccgccgggaccccggaccccggccctgcgcccgcgtcgtctctgctgtaccttcccgccaactacctcggtgcgcgcmcggctcgcaggccccgccagaaggcccgtggcaacggcgaatacggcgcgtgcgtcmcggccccagggtc

SEQ ID NO: 2 - DNA, 9046 bp, Homo sapiens
ccttgtttgtgtgctaggctgggggggagagagggcgagagagagcgggcgagagtgggcaagcaggacgccgggctgagtgctaactgcgggacgcagagagtgcggaggggagtcgggtcggagagaggcggcaggggccagaacagtggcagggggcccggggcgcacgggctgaggcgacccccagccccctcccgtccgcacacacccccaccgcggtccagcagccgggccggcgtcgacgctaggggggaccattacataacccgcgccccggccgtcttctcccgccgccgcggcgcccgaactgagcccggggcgggcgctccagcactggccgccggcgtggggcgtagcagcggccgtattattatttcgcggaaaggaaggcgaaggaggggagcgccggcgcgaggaggggccgcctgcgcccgccgccggagcggggcctcctcggtgggctccgcgtcggcgcgggcgtgcgggcggcgctgctcggcccggccccctcggccctctggtccggccagctccgctcccggcgtccttgccgcgcctccgccggccgccgcgcgatgtgaggcggcggcgccagcctggctctcggctcgggcgagttctctgcggccattaggggccggtgcggcggcggcgcggagcgcggcggcaggaggagggttcggagggtgggggcgcaggcccgggagggggcaccgggaggaggtgagtgtctcttgtcgcctcctcctctccccccttttcgcccccgcctccttgtggcgatgagaaggaggaggacagcgccgaggaggaagaggttgatggcggcggcggagctccgagagacctcggctgggcaggggccggccgtggcgggccggggactgcgcctctagagccgcgagttctcgggaattcgccgcagcggaccggcctcggcgaatttgtgctcttgtgccctcctccgggcttgggccaggccggcccctcgcacttgcccttaccttttctatcgagtccgcatccctctccagccactgcgacccggcgaagagaaaaaggaacttcccccaccccctcgggtgccgtcggagccccccagcccacccctgggtgcggcgcggggaccccgggccgaagaagagatttcctgaggattctggttttcctcgcttgtatctccgaaagaattaaaaatggccgagaatgtggtggaaccggggccgccttcagccaagcggcctaaactctcatctccggccctctcggcgtccgccagcgatggcacagattttggctctctatttgacttggagcacgacttaccagatgaattaatcaactctacagaattgggactaaccaatggtggtgatattaatcagcttcagacaagtcttggcatggtacaagatgcagcttctaaacataaacagctgtcagaattgctgcgatctggtagttcccctaacctcaatatgggagttggtggcccaggtcaagtcatggccagccaggcccaacagagcagtcctggattaggtttgataaatagcatggtcaaaagcccaatgacacaggcaggcttgacttctcccaacatggggatgggcactagtggaccaaatcagggtcctacgcagtcaacaggtatgatgaacagtccagtaaatcagcctgccatgggaatgaacacagggacgaatgcgggcatgaatcctggaatgttggctgcaggcaatggacaagggataatgcctaatcaagtcatgaacggttcaattggagcaggccgagggcgacaggatatgcagtacccaaacccaggcatgggaagtgctggcaacttactgactgagcctcttcagcagggctctccccagatgggaggacaaacaggattgagaggcccccagcctcttaagatgggaatgatgaacaaccccaatccttatggttcaccatatactcagaatcctggacagcagattggagccagtggccttggtctccagattcagacaaaaactgtactatcaaataacttatctccatttgctatggacaaaaaggcagttcctggtggaggaatgcccaacatgggtcaacagccagccccgcaggtccagcagccaggtctggtgactccagttgcccaagggatgggttctggagcacatacagctgatccagagaagcgcaagctcatccagcagcagcttgttctccttttgcatgctcacaagtgccagcgccgggaacaggccaatggggaagtgaggcagtgcaaccttccccactgtcgcacaatgaagaatgtcctaaaccacatgacacactgccagtcaggcaagtcttgccaagtggcacactgtgcatcttctcgacaaatcatttcacactggaagaattgtacaagacatgattgtcctgtgtgtctccccctcaaaaatgctggtgataagagaaatcaacagccaattttgactggagcacccgttggacttggaaatcctagctctctaggggtgggtcaacagtctgcccccaacctaagcactgttagtcagattgatcccagctccatagaaagagcctatgcagctcttggactaccctatcaagtaaatcagatgccgacacaaccccaggtgcaagcaaagaaccagcagaatcagcagcctgggcagtctccccaaggcatgcggcccatgagcaacatgagtgctagtcctatgggagtaaatggaggtgtaggagttcaaacgccgagtcttctttctgactcaatgttgcattcagccataaattctcaaaacccaatgatgagtgaaaatgccagtgtgccctccctgggtcctatgccaacagcagctcaaccatccactactggaattcggaaacagtggcacgaagatattactcaggatcttcgaaatcatcttgttcacaaactcgtccaagccatatttcctacgccggatcctgctgctttaaaagacagacggatggaaaacctagttgcatatgctcggaaagttgaaggggacatgtatgaatctgcaaacaatcgagcggaatactaccaccttctagctgagaaaatctataagatccagaaagaactagaagaaaaacgaaggaccagactacagaagcagaacatgctaccaaatgctgcaggcatggttccagtttccatgaatccagggcctaacatgggacagccgcaaccaggaatgacttctaatggccctctacctgacccaagtatgatccgtggcagtgtgccaaaccagatgatgcctcgaataactccacaatctggtttgaatcaatttggccagatgagcatggcccagccccctattgtaccccggcaaacccctcctcttcagcaccatggacagttggctcaacctggagctctcaacccgcctatgggctatgggcctcgtatgcaacagccttccaaccagggccagttccttcctcagactcagttcccatcacagggaatgaatgtaacaaatatccctttggctccgtccagcggtcaagctccagtgtctcaagcacaaatgtctagttcttcctgcccggtgaactctcctataatgcctccagggtctcaggggagccacattcactgtccccagcttcctcaaccagctcttcatcagaattcaccctcgcctgtacctagtcgtacccccacccctcaccatactcccccaagcataggggctcagcagccaccagcaacaacaattccagcccctgttcctacaccaccagccatgccacctgggccacagtcccaggctctacatccccctccaaggcagacacctacaccaccaacaacacaacttccccaacaagtgcagccttcacttcctgctgcaccttctgctgaccagccccagcagcagcctcgctcacagcagagcacagcagcgtctgttcctaccccaaacgcaccgctgcttcctccgcagcctgcaactccactttcccagccagctgtaagcattgaaggacaggtatcaaatcctccatctactagtagcacagaagtgaattctcaggccattgctgagaagcagccttcccaggaagtgaagatggaggccaaaatggaagtggatcaaccagaaccagcagatacgcagccggaggatatttcagagtctaaagtggaagactgtaaaatggaatctaccgaaacagaagagagaagcactgagttaaaaactgaaataaaagaggaggaagaccagccaagtacttcagctacccagtcatctccggctccaggacagtcaaagaaaaagattttcaaaccagaagaactacgacaggcactgatgccaacattggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggaccgctttgtctacacctgcaatgaatgcaagcaccatgtggagacacgctggcactgtactgtctgtgaggattatgacttgtgtatcacctgctataacactaaaaaccatgaccacaaaatggagaaactaggccttggcttagatgatgagagcaacaaccagcaggctgcagccacccagagcccaggcgattctcgccgcctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgcctaaacatcaagcagaagctccggcagcaacagctgcagcaccgactacagcaggcccaaatgcttcgcaggaggatggccagcatgcagcggactggtgtggttgggcagcaacagggcctcccttcccccactcctgccactccaacgacaccaactggccaacagccaaccaccccgcagacgccccagcccacttctcagcctcagcctacccctcccaatagcatgccaccctacttgcccaggactcaagctgctggccctgtgtcccagggtaaggcagcaggccaggtgacccctccaacccctcctcagactgctcagccaccccttccagggcccccacctacagcagtggaaatggcaatgcagattcagagagcagcggagacgcagcgccagatggcccacgtgcaaatttttcaaaggccaatccaacaccagatgcccccgatgactcccatggcccccatgggtatgaacccacctcccatgaccagaggtcccagtgggcatttggagccagggatgggaccgacagggatgcagcaacagccaccctggagccaaggaggattgcctcagccccagcaactacagtctgggatgccaaggccagccatgatgtcagtggcccagcatggtcaacctttgaacatggctccacaaccaggattgggccaggtaggtatcagcccactcaaaccaggcactgtgtctcaacaagccttacaaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacgacagcaaatgatgcaacagcagcagcaacagggagcagggccaggaataggccctggaatggccaaccataaccagttccagcaaccccaaggagttggctacccaccacagccgcagcagcggatgcagcatcacatgcaacagatgcaacaaggaaatatgggacagataggccagcttccccaggccttgggagcagaggcaggtgccagtctacaggcctatcagcagcgactccttcagcaacagatggggtcccctgttcagcccaaccccatgagcccccagcagcatatgctcccaaatcaggcccagtccccacacctacaaggccagcagatccctaattctctctccaatcaagtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgataactcagacttgaattcaaacctctcacagagtacactagacatacactagagacaccttgtattttgggagcaaaaaaattattttctcttaacaagactttttgtactgaaaacaatttttttgaatctttcgtagcctaaaagacaattttccttggaacacataagaactgtgcagtagccgtttgtggtttaaagcaaacatgcaagatgaacctgagggatgatagaatacaaagaatatatttttgttatgggctggttaccaccagcctttcttcccctttgtgtgtgtggttcaagtgtgcactgggaggaggctgaggcctgtgaagccaaacaatatgctcctgccttgcacctccaataggttttattattttttttaaattaatgaacatatgtaatattaatgaacatatgtaatattaatagttattatttactggtgcagatggttgacatttttccctattttcctcactttatggaagagttaaaacatttctaaaccagaggacaaaaggggttaatgttactttgaaattacattctatatatatataaatatatataaatatatattaaaataccagttttttttctctgggtgcaaagatgttcattcttttaaaaaatgtttaaaaaaaa

SEQ ID NO: 3 - DNA, 1918 bp, Homo sapiens
cggtgccgcggggatggcgggagccggagctggagccggagctcgcggcggagcggcggcgggggtcgaggctcgagctcgcgatccaccgcccgcgcaccgcgcacatcctcgccaccctcggcctgcggctcagccctcggcccgcaggatggatggcgggtcagggggcctggggtctggggacaacgccccgaccactgaggctcttttcgtggcactgggcgcgggcgtgacggcgctcagccatcccctgctctacgtgaagctgctcatccaggtgggtcatgagccgatgccccccacccttgggaccaatgtgctggggaggaaggtcctctatctgccgagcttcttcacctacgccaagtacatcgtgcaagtggatggtaagatagggctgttccgaggcctgagtccccggctgatgtccaacgccctctctactgtgactcggggtagcatgaagaaggttttccctccagatgagattgagcaggtttccaacaaggatgatatgaagacttccctgaagaaagttgtgaaggagacctcctacgagatgatgatgcagtgtgtgtcccgcatgttggcccaccccctgcatgtcatctcaatgcgctgcatggtccagtttgtgggacgggaggccaagtacagtggtgtgctgagctccattgggaagattttcaaagaggaagggctgctgggattcttcgttggattaatccctcacctcctgggcgatgtggttttcttgtggggctgtaacctgctggcccacttcatcaatgcctacctggtggatgacagcgtgagtgacaccccaggggggctgggaaacgaccagaatccaggttcccagttcagccaggccctggccatccggagctataccaagttcgtgatggggattgcagtgagcatgctgacctaccccttcctgctagttggcgacctcatggctgtgaacaactgcgggctgcaagctgggctccccccttactccccagtgttcaaatcctggattcactgctggaagtacctgagtgtgcagggccagctcttccgaggctccagcctgcttttccgccgggtgtcatcaggatcatgctttgccctggagtaacctgaatcatctaaaaaacacggtctcaacctggccaccgtgggtgaggcctgaccaccttgggacacctgcaagacgactccaacccaacaacaaccagatgtgctccagcccagccgggcttcagttccatatttgccatgtgtctgtccagatgtggggttgagcgggggtggggctgcacccagtggattgggtcacccggcagacctagggaaggtgaggcgaggtggggagttggcagaatccccatacctcgcagatttgctgagtctgtcttgtgcagagggccagagaatggcttatgggggcccaggttggatggggaaaggctaatggggtcagaccccaccccgtctacccctccagtcagcccagcgcccatcctgcagctcagctgggagcatcattctcctgctttgtacatagggtgtggtcccctggcacgtggccaccatcatgtctaggcctatgctaggaggcaaatggccagctctgcctgtgtttttctcaacactacttttctgatatgagggcagcacctgcctctgaatgggaaatcatgcaactactcagaatgtgtcctcctcatctaatgctcatctgtttaatggtgatgcctcgcgtacaggatctggttacctgtgcagttgtgaatacccagaggttgggcagatcagtgtctctagtcctacccagttttaaagttcatggtaagatttgacctcatctcccgcaaataaatgtattggtgatttggaaaaaaaaaaaaaaaaaaaaaaaaaaaaa

SEQ ID NO: 4 - DNA, 1254 bp, Homo sapiens
atgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagggagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcggtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagagcctgtctgcctcctgctgccatcaacctgctgtctatagctcccatggcacccaggctgttcatgccaaagggtgcctgcaggcccacggcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattt

SEQ ID NO: 5 - DNA, 1380 bp, Homo sapiens
cgagcggcggcggagcaggcatttccagcagtgaggagacagccagaagcaagctattggagctgaaggaacctgagacagaagctagtcccccctctgaattttactgatgaagaaactgaggccacagagctaaagtgacttttcccaaggtcgcccagcgaggacgtgggacttctcagacgtcaggagagtgatgtgagggagctgtgtgaccatagaaagtgacgtgttaaaaaccagcgctgccctctttgaaagccagggagcatcattcatttagcctgctgagaagaagaaaccaagtgtccgggattcagacctctctgcggccccaagtgttcgtggtgcttccagaggcagggctatgctcacattcatggcctctgacagcgaggaagaagtgtgtgatgagcggacgtccctaatgtcggccgagagccccacgccgcgctcctgccaggagggcaggcagggcccagaggatggagagaacactgcccagtggagaagccaggagaacgaggaggacggtgaggaggaccctgaccgctatgtctgtagtggggttcccgggcggccgccaggcctggaggaagagctgaccctcaaatacggagcgaagcacgtgatcatgctgtttgtgcctgtcactctgtgcatgatcgtggtggtagccaccatcaagtctgtgcgcttctacacagagaagaatggacagctcatctacacgacattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatctgccatggtgtggacggttggcatggcgaagctggacccctcctctcagggtgccctccagctcccctacgacccggagatggaagaagactcctatgacagttttggggagccttcataccccga

SEQ ID NO: 6 - DNA, 3571 bp, Homo sapiens
  misc_feature (2506)..(2506): unknown nucleotide
  misc_feature (3393)..(3393): unknown nucleotide
ctcagcaggtcaggacatttggtaggggaaggttgaaagacaaaagcagcaggccttgggttctcagccttttaaaaactattattaaatatatatttttaaaatttagtggttagagcttttagtaatgtgcctgtattacatgtagagagtattcgtcaaccaagaggagttttaaaatgtcaaaaccgggaaaacctactctaaaccatggcttggttcctgttgatcttaaaagtgcaaaagagcctctaccacatcaaaccgtgatgaagatatttagcattagcatcattgcccaaggcctccctttttgtcgaagacggatgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaagtgaagagcagaaaagatgaatttgtccaattgtgtagacgatatactccctgcccttccccctgcaaaaagaaaaaaagaaaagaaaaagaaaaaaatcccttgttctcagtggtgcaggatgttggcttggggcaacagattgaaaagacctacagactaagaaggaaaagaagaagagatgacaaaccataactgacaagagaggcgtctgctgtctcatcacataaaaggcttcacttttgactgagggcagctttgcaaaatgagactttctaaatcaaaccaggtgcaattatttctttattttcttctccagtggtcattggggcagtgttaatgctgaaacatcattacagattctgctagcctgttcttttaccactgacagctagacacctagaaaggaactgcaataatatcaaaacaagtactggttgactttctaattagagagcatctgcaacaaaaagtcatttttctggagtggaaaagcttaaaaaaattactgtgaattgtttttgtacagttatcatgaaaagcttttttttttattttttngccaaccattgccaatgtcaatcaatcacagtattagcctctgttaatctatttactgttgcttccatatacattcttcaatgcatatgttgctcaaaggtggcaagttgtcctgggttctgtgagtcctgagatggatttaattcttgatgctggtgctagaagtaggtcttcaaatatgggattgttgtcccaaccctgtactgtactcccagtggccaaacttatttatgctgctaaatgaaagaaagaaaaaagcaaattattttttttattttttttctgctgtgacgttttagtcccagactgaattccaaatttgctctagtttggttatggaaaaaagactttttgccactgaaacttgagccatctgtgcctctaagaggctgagaatggaagagtttcagataataaagagtgaagtttgcctgcaagtaaagaattgagagtgtgtgcaaagcttattttcttttatctgggcaaaaattaaaacacattccttggaacagagctattacttgcctgttctgtggagaaacttttctttttgagggctgtggtgaatggatgaacgtacatcgtaaaactgacaaaatattttaaaaatatataaaacacaaaattaaaataaagttgctggtcagtcttagtgttttacagtatttgggaaaacaactgttacagttttattgctctgagtaactgacaaagcagaaactattcagtttttgtagtaaaggcgtcacatgcaaacaaacaaaatgaatgaaacagtcaaatggtttgcctcattctccaagagccacaactcaagctgaactgtgaaagtggtttaacactgtatcctaggcgatcttttttcctccttctgtttatttttttgnttgttttatttatagtctgatttaaaacaatcagattcaagttggttaattttagttatgtaacaacctgacatgatggaggaaaacaacctttaaagggattgtgtctatggtttgattcacttagaaattttattttcttataacttaagtgcaataaaatgtgttttttcatgtt

SEQ ID NO: 7 - DNA, 1257 bp, Homo sapiens
gtcgaccgcctgcccaggcccggggagggaggaggcgggcgtcagggtgctgcgccccgctcggcgtccgagcttccggccgggctgtgccccgcgcggtcttcgccgggatgaagcgcccctgcgaggagacgacctccgagagcgacatggacgagaccatcgacgtggggagcgagaacaattactcggggcaaagtactagctctgtgattagattgaattctccaacaacaacatctcagattatggcaagaaagaaaaggagagggattatagagaaaaggcgtcgggatcggataaataacagtttatctgagttgagaagacttgtgccaactgcttttgaaaaacaaggatctgcaaagttagaaaaagctgaaatattgcaaatgacagtggatcatttgaagatgcttcaggcaacagggggtaaaggctactttgacgcacacgctcttgccatggacttcatgagcataggattccgagagtgcctaacagaagttgcgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcggccacagccatcagcccgcccttgtcagtatcagccacgtccagtcctcagcagaccagcagtggaacaaacaataaaccttaccgaccctgggggacagaagttggagctttttaaatttttcttgaacttcttgcaatagtaactgaatgtcctccatttcagagtcagcttaaaacctctgcaccctgaaggtagccatacagatgccgacagatccacaaaggaacaataaagctatttgagacac

SEQ ID NO: 8 - DNA, 2223 bp, Homo sapiens
tcagtgtgtgcggaacgcaagcagccgagagcggagaggcgccgctgtagttaactcctccctgcccgccgcgccgaccctccccaggaacccccagggagccagcatgaagcgagctcaccccgagtacagctcctcggacagcgagctggacgagaccatcgaggtggagaaggagagtgcggacgagaatggaaacttgagttcggctctaggttccatgtccccaactacatcttcccagattttggccagaaaaagacggagaggaataattgagaagcgccgacgagaccggatcaataacagtttgtctgagctgagaaggctggtacccagtgcttttgagaagcagggatctgctaagctagaaaaagccgagatcctgcagatgaccgtggatcacctgaaaatgctgcatacggcaggagggaaaggttactttgacgcgcacgcccttgctatggactatcggagtttgggatttcgggaatgcctggcagaagttgcgcgttatctgagcatcattgaaggactagatgcctctgacccgcttcgagttcgactggtttcgcatctcaacaactacgcttcccagcgggaagccgcgagcggcgcccacgcgggcctcggacacattccctgggggaccgtcttcggacatcacccgcacatcgcgcacccgctgttgctgccccagaacggccacgggaacgcgggcaccacggcctcacccacggaaccgcaccaccagggcaggctgggctcggcacatccggaggcgcctgctttgcgagcgccccctagcggcagcttcggaccggtgctccctgtggtcacctccgcctccaaactgtcgctgcctctgctctcctcagtggcctccctgtcggccttccccttctctttcggctccttccacttactgtctcccaatgcactgagcccttcagcacccacgcaggctgcaaaccttggcaagccctatagaccttgggggacggagatcggagctttttaaagaactgatgtagaatgagggaggggaaagtttaaaatcccagctgggctggactgttgccaacatcaccttaaagtcgtcagtaaaagtaaaaaggaaaaaggtacactttcagataattttttttttaaagactaaaggtttgttggtttacttttatcttttttaatgtttttttcatcatgtcatgtattagcagtttttaaaaactagttgttaaattttgttcaagacattaaattgaaatagtgagtataagccaacactttgtgataggtttgtactgtgcctaatttactttgtaaaccagaatgattccgtttttgcctcaaaatttggggaatcttaacatttaggtatttttggtctgtttttctccttgtatagttatggtctgtttttagaattaattttccaaaccactatgcttaatgttaacatgattctgtttgttaatattttgacagattaaggtgttgtataaataatattcttttggggggaggggaactatattgaattttatatttctgagcaaagcgttgacaaatcagatgatcagctttatccaagaaagaagactagtaaattgtctgcctcctatagcagaaaggtgaatgtacaaactgttggtggcctgaatccatctgaccagctgctggtatctgccaggactggcagttctgatttagttaggaggaccgctgataggttaggtctcatttggagtgttggtggaaaggaaactgaaggtaattgaatagaatacgcctgcatttaccagccccagcaacacaaagaatttttaatcacacggatctcaaattcacaaatgttaacatggataagtgatcatggtgtgcgagtggtcaattgagtagtacagtggaaactgttaaatgcataacctaattttcctgggactgccatattttcttttaactggaaatttttatgtgagttttccttttggtgcatggaactgtggttgccaaggtatttaaaagggctttcctgcctccttctctttgatttatttaatttgatttgggctataaaatatcatttttcaggtttattcttttagcaggtgtagttaaacgacctccactgaactgggtttgacctctgttgtactgatgtgttgtgactaaataaaaaagaaagaacaaagtaaaaaaaaaaaaaaaaaaaaaaaaaaa

SEQ ID NO: 9 - DNA, 1471 bp, Homo sapiens
atcacacaggatccggagctggtgctgataacagcggaatcccccgtctacctctctccttggtcctggaacagcgctactgatcaccaagtagccacaaaatataataaaccctcagcacttgctcagtagttttgtgaaagtctcaagtaaaagagacacaaacaaaaaattctttttcgtgaagaactccaaaaataaaattctctagagataaaaaaaaaaaaaaaaggaaaatgccagctgatataatggagaaaaattcctcgtccccggtggctgctaccccagccagtgtcaacacgacaccggataaaccaaagacagcatctgagcacagaaagtcatcaaagcctattatggagaaaagacgaagagcaagaataaatgaaagtctgagccagctgaaaacactgattttggatgctctgaagaaagatagctcgcggcattccaagctggagaaggcggacattctggaaatgacagtgaagcacctccggaacctgcagcgggcgcagatgacggctgcgctgagcacagacccaagtgtgctggggaagtaccgagccggcttcagcgagtgcatgaacgaggtgacccgcttcctgtccacgtgcgagggcgttaataccgaggtgcgcactcggctgctcggccacctggccaactgcatgacccagatcaatgccatgacctaccccgggcagccgcaccccgccttgcaggcgccgccaccgcccccaccgggacccggcggcccccagcacgcgccgttcgcgccgccgccgccactcgtgcccatccccgggggcgcggcgccccctcccggcggcgccccctgcaagctgggcagccaggctggagaggcggctaaggtgtttggaggcttccaggtggtaccggctcccgatggccagtttgctttcctcattcccaacggggccttcgcgcacagcggccctgtcatccccgtctacaccagcaacagcggcacctccgtgggccccaacgcagtgtcaccttccagcggcccctcgcttacggcggactccatgtggaggccgtggcggaactgagggggctcaggccacccctcctcctaaactccccaacccacctctcttccctccggactctaaacaggaacttgaatactgggagagaagaggacttttttgattaagtggttactttgtgtttttttaatttctaagaagttactttttgtagagagagctgtattaagtgactgaccatgcactatatttgtatatattttatatgttcatattggattgcgcctttgtattataaaagctcagatgacatttcgttttttacacgagatttcttttttatgtgatgccaaagatgtttgaaaatgctcttaaaatatcttcctttggggaagtttatttgagaaaatataataaaagaaaaaagtaaaggcaaaaaaaaaaaaaaaaaa

SEQ ID NO: 10 - DNA, 642 bp, Homo sapiens
  misc_feature (400)..(400): unknown nucleotide
gcgcggggacactcgtgcgactggggcaaggtgcccacgggcttctctcctaatttgcctcacgtaacagttgagaccccagagggcagcaaaactgggttcgaattggagagccgtccaggcacagacaaattcattcatctgcccagtgcccgctaactgcggttccaggcgccggcgacgcaattccagagctgccaccgcttccccgcggagcatggggctgcctcgccgggcaggggacgcggcggagctgcgcaagagcctgaagccgctgctggagaagcgccggcgcgcgcgcatcaaccagagcctgagccagcttaaggggctcatcctgccgctgctgggccgggaggatgcttctggctggcacacctggcttcccctccatgctcangaactgcttcctactctacatccaggctcctgagcagcccccagcttaagcttcagcagctccagggtaaatccatctccgcccagagctgactccggatctgccccagtgacaataacaagataaatgccttccattctcttcgtacttacaggatgtaatccacatatttacacagtgtttcatttgcggtaatgtttgtatagtttcttgttccttcgtacctatctgggaagcctgtg

SEQ ID NO: 11 - DNA, 666 bp, Homo sapiens
atggccgcagacacgccggggaaaccgagcgcctcgccgatggcaggagcgccggccagcgccagccggaccccagacaagccccggagcgcggccgagcaccgcaagtcctccaagccggtcatggagaagcggcgccgagcgcgtattaacgagagcctcgctcagctcaaaaccctcatcctggacgccctcagaaaagagagctcccgccactcgaagctggagaaggcggacatcctggagatgaccgtgagacacctgcggagcctgcgtcgcgtgcaggtgacggccgcgctcagcgccgacccggccgttctgggcaagtaccgcgccggcttccacgagtgtctggcggaggtgaaccgcttcctggccggctgcgagggcgtcccggccgacgtgcgctcccgcctgctgggccacctggcagcctgcctgcgccagctgggaccctcccgccgcccggcctcgctgtccccggctgcccccgcagaggccccagcgcccgaggtctacgcgggccgcccgctgctgccatcgctcggcggccccttccctctgctcgcgccgccgctgctgccgggtctgacccgggcgctgcccgccgcccccagggcggggccgcagggcccgggtgggccctggaggccgtggctgcgctga

SEQ ID NO: 12 - DNA, 2103 bp, Homo sapiens
cccaggaacccccagggagccagcatgaagcgagctcaccccgagtacagctcctcggacagcgagctggacgagaccatcgaggtggagaaggagagtgcggacgagaatggaaacttgagttcggctctaggttccatgtccccaactacatcttcccagattttggccagaaaaagacggagaggaataattgagaagcgccgacgagaccggatcaataacagtttgtctgagctgagaaggctggtacccagtgcttttgagaagcagggatctgctaagctagaaaaagccgagatcctgcagatgaccgtggatcacctgaaaatgctgcatacggcaggagggaaaggttactttgacgcgcacgcccttgctatggactatcggagtttgggatttcgggaatgcctggcagaagttgcgcgttatctgagcatcattgaaggactagatgcctctgacccgcttcgagttcgactggtttcgcatctcaacaactacgcttcccagcgggaagccgcgagcggcgcccacgcgggcctcggacacattccctgggggaccgtcttcggacatcacccgcacatcgcgcacccgctgttgctgccccagaacggccacgggaacgcgggcaccacggcctcacccacggaaccgcaccaccagggcaggctgggctcggcacatccggaggcgcctgctttgcgagcgccccctagcggcagcctcggaccggtgctccctgtggtcacctccgcctccaaactgtcgccgcctctgctctcctcagtggcctccctgtcggccttccccttctctttcggctccttccacttactgtctcccaatgcactgagcccttcagcacccacgcaggctgcaaaccttggcaagccctatagaccttgggggacggagatcggagctttttaaagaactgatgtagaatgagggaggggaaagtttaaaatcccagctgggctggactgttgccaacatcaccttaaagtcgtcagtaaaagtaaaaaggaaaaaggtacactttcagataattttttttttaaagactaaaggtttgttggtttacttttatcttttttaatgtttttttcatcatgtcatgtattagcagtttttaaaaactagttgttaaattttgttcaagacattaaattgaaatagtgagtataagccaacactttgtgataggtttgtactgtgcctaatttactttgtaaaccagaatgattccgtttttgcctcaaaatttggggaatcttaacatttagtatttttggtctgtttttctccttgtatagttatggtctgtttttagaattaattttccaaaccactatgcttaatgttaacatgattctgtttgttaatattttgacagattaaggtgttgtataaataatattcttttggggggaggggaactatattgaattttatatttctgagcaaagcgttgacaaatcagatgatcagctttatccaagaaagaagactagtaaattgtctgcctcctatagcagaaaggtgaatgtacaaactgttggtggccctgaatccatctgaccagctgctggtatctgccaggactggcagttctgatttagttaggagagagccgctgataggttaggtctcatttggagtgttggtggaaaggaaactgaaggtaattgaatagaatacgcctgcatttaccagccccagcaacacaaagaatttttaatcacacggatctcaaattcacaaatgttaacatggataagtgatcatggtgtgcgagtggtcaattgagtagtacagtggaaactgttaaatgcataacctaattttcctgggactgccatattttcttttaactggaaatttttatgtgagttttccttttggtgcatggaactgtggttgccaaggtatttaaaagggctttcctgcctccttctctttgatttatttaatttgatttgggctataaaatatcatttttcaggtttattcttttagcaggtgtagttaaacgacctccactgaactgggtttgacctctgttgtactgatgtgttgtgactaaataaaaaa

SEQ ID NO: 13 - DNA, 2223 bp, Homo sapiens; identical to SEQ ID NO: 8

SEQ ID NO: 14 - DNA, 2533 bp, Homo sapiens
tcggcgtccgagcttccggccgggctgtgccccgcgcggtcttcgccgggatgaagcgcccctgcgaggagacgacctccgagagcgacatggacgagaccatcgacgtggggagcgagaacaattactcggggcaaagtactagctctgtgattagattgaattctccaacaacaacatctcagattatggcaagaaagaaaaggagagggattatagagaaaaggcgtcgggatcggataaataacagtttatctgagttgagaagacttgtgccaactgcttttgaaaaacaaggatctgcaaagttagaaaaagctgaaatattgcaaatgacagtggatcatttgaagatgcttcaggcaacagggggtaaaggctactttgacgcacacgctcttgccatggacttcatgagcataggattccgagagtgcctaacagaagttgcgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcggccacagccatcagcccgcccttgtcagtatcagccacgtccagtcctcagcagaccagcagtggaacaaacaataaaccttaccgaccctgggggacagaagttggagctttttaaatttttcttgaacttcttgcaatagtaactgaatgtcctccatttcagagtcagcttaaaacctctgcaccctgaaggtagccatacagatgccgacagatccacaaaggaacaataaagctatttgagacacaaacctcacgagtggaaatgtggtattctcttttttttctctcccttttttgtttggttcaaggcagctcggtaactgacatcagcaacttttgaaaacttcacacttgttaccatttagaagtttcctggaaaatatatggaccgtaccatccagcagtgcatcagtatgtctgaattggggaagtaaaatgccctgactgaattctcttgagactagatgggacatacatatatagagagagagtgagagagtcgtgtttcgtaagtgcctgagcttaggaagttttcttctggatatataacattgcacaagggaagacgagtgtggaggataggttaagaaaggaaagggacagaagtcttgcaataggctgcagacattttaataccatgccagagaagagtattctgctgaaaccaacaggttttactggtcaaaatgactgctgaaaataattttcaagttgaaagatctagttttatcttagtttgccttctttgtacagacatgccaagaggtgacatttagcagtgcattggtataagcaattatttcatcagttctcagattaacaagcatttctgctctgcctgcaggcccccaggcacttttttttttggatggctcaaaatatggtgctgctttatataaaccttacatttatatagtgcacctatgagcagttgcctaccatgtgtccaccagaggctatttaattcatgccaacttgaaaactctccagtttgtaggagtttggtttaatttattcagtttcattaggactatttttatatatttatcctcttcattttctcctaatgatgcaacatctattcttgtcaccctttgggagaagttacatttctggaggtgatgaagcaaggagggagcactaggaagagaaaagctacaatttttaaagctctttgtcaagttagtgattgcatttgatcccaaaacaagatgaatgtatgcaatgggatgtacataagttatttttgcccatgcctaaactagtgctatgtaatggggttgtggttttgtttttttcgatttcgtttaatgacaaaataatctcttaatatgctgaaatcaagcacgtgagagtttttgtttaaaagataagagacacagcatgtattatgcacttcatttctctactgtgtggagaaagcaataaacattatgagaatgttaaacgttatgcaaaattatacttttaaatatttgttttgaaattactgtacctagtcttttttgcattactttgtaacctttttctatgcaagagtctttacataccactaattaaatgaagtcctttttgactatt

SEQ ID NO: 15 - DNA, 3416 bp, Homo sapiens
accaggcagcctgcgttcgccatgaagcgacccaaggagccgagcggctccgacggggagtccgacggacccatcgacgtgggccaagagggccagctgagccagatggccaggccgctgtccacccccagctcttcgcagatgcaagccaggaagaaacgcagagggatcatagagaaacggcgtcgagaccgcatcaacagtagcctttctgaattgcgacgcttggtccccactgcctttgagaaacagggctcttccaagctggagaaagccgaggtcttgcagatgacggtggatcacttgaaaatgctccatgccactggtgggacaggattctttgatgcccgagccctggcagttgacttccggagcattggttttcgggagtgcctcactgaggtcatcaggtacctgggggtccttgaagggcccagcagccgtgcagaccccgtccggattcgccttctctcccacctcaacagctacgcagccgagatggagccttcgcccacgcccactggccctttggccttccctgcctggccctggtctttcttccatagctgtccagggctgccagccctgagcaaccagctcgccatcctgggaagagtgcccagccctgtcctccccggtgtctcctctcctgcttaccccatcccagccctccgaaccgctccccttcgcagagccacaggcatcatcctgccagcccggaggaatgtgctgcccagtcgaggggcatcttccacccggagggcccgccccctagagaggccagcgacccctgtgcctgtcgcccccagcagcagggctgccaggagcagccacatcgctcccctcctgcagtcttcctccccaacaccccctggtcctacagggtcggctgcttacgtggctgttcccacccccaactcatcctccccagggccagctgggaggccagcgggagccatgctctaccactcctgggtctctgaaatcactgaaatcggggctttctgagctgccccttcaccaccccgccccaaggaataaggaaggttcttttaccaggagcccaaaaaagggcactgccttttctgctttgcttcgtggactggctcatatgtgaaggcacgttctccagccatcagaggccccctcctcctccaacccatctctccttctcactgttatcccagcttatccacccagctctcctggagctgttctggtctcagaggcttggttccatttctcacctgaacagatgagtcctgggagagaccctcagagatccgcccagacccctctcctgccctctgcacaccagcagcaggcatgaaccttgggtctgggaaaaagctttaacctgcagggcaccaggacccaaggcaggctgttccttggggcggtcagaccccagtcaggagcaatgactgactggctgcagccttcccacgccaagaggctggaacatagtgtctgcctcgcttcctggagatagtaactgagcaggggctacaaagaggtctcctgggaaccctgtctgccccttcccacctgtccttgggccacaccatcacactgaaccacaggacagaccctttctccaccacagccaaggcctggagactgggggcccagcagagcctgctcccaccctcctcccagcagcagacacccaccctctcactgactaacaggtccctgcacacagctggcctggtaaacccagctgggaggtttctaggcagcagcaaaactctgtgacagggtgtcctcacaccaggccttggacagctctcccagacaggagccagggttgagcaatggagagcccagcccccacgtcttacagtcgccatcctccaggcgtgtggtccctccccattgggtgcacagtgcagaggggccgtggccccatgtgatggtgcgcagagaggaacctcttgggattcagcaccagacgtctgtgctgcctggtttgcatccggctcacagagcccagactgctggaacagccaaggactgtcaggctggacaaaaataactgcaaggaggggcaagagaaaggatgattcgaggcaccttggcccttcaaggtcatgcagtgggtcgagcgcctgagatcctgttcaccaggactccacagagctggctctgctcagaagccatttcattccccggctccaccctaggccactttttctaacagaggaaacaaatggtccagcagtcgttcccagcagaacagcggagcctggactgacacccagtgggaccagtgttgccacaccagttgataaaatgcagaaacccttctgtactcgttggtaaatatctactcccccaagtgactccaggtgccccccaccgcctggcacttcccccaggactcctacgatctggttactgcctggccgatccaaggctgtggagtcccagagccagcagttcactggtgctcattccacactggttagatacttcagttgtcacccctgggaagattctcccacctcctccctttgatggaaccaccctccccagaggctgcattgaggagactccacagactgaaaagtgagtttgcagaaaccttggggaaaagggccctttcaaagaagtggataagagggaggagatcattgagtgacccagaaagctcttttgaaaagacagactcctcaaggagagataaagaggaaagcacctctttcattttttagtgtgagctaattccatcagactgctgtcctcctggacccatctgagatgtgcagtagcaaggagaggggggatcattttagagagtgggtcattggcagggagtgctccggagggaggcagaggggagactgtggtagaaggaagacagaactcacacatgctcccaggattggggacagggacagaggaggtaacagaaggcaaaggccagtttccccgttatcatgaaggggcccactcaggacaggaacaaggacaactcctcctcctcctcctcctctcctgctgctcctgggataccaggtcagtgatgtagtcttgcagtttggcaacttcctagcctgagaatccctagtggggctgtgggaaacacatttccacgttgcaagcatgcaactccaaagaatctgtgatgccactgaaatgagatgggaatgatccagctctttcagcatcttggttgaacttgctttcattgtccctgggatattgtggaaggaaaggtgactgtgtgatctgattctgtggtcaaggacttgcatcttgtgtttctatccccaagccttcctggtgtctccaactcctaccccattgcatgggttgttgcggacatccaataaagatttttttagtgcttctggaaaaaaaaaaaaaaaaaaaaaaaaaaaaaaaaaaaaaaaaaaaaaaaaaaaaaaacc

SEQ ID NO: 16 - DNA, 2533 bp, Homo sapiens; identical to SEQ ID NO: 14

SEQ ID NO: 17 - DNA, 3416 bp, Homo sapiens; identical to SEQ ID NO: 15

SEQ ID NO: 18 - DNA, 915 bp, Homo sapiens
atgaagcgagctcaccccgagtacagctcctcggacagcgagctggacgagaccatcgaggtggagaaggagagtgcggacgagaatggaaacttgagttcggctctaggttccatgtccccaactacatcttcccagattttggccagaaaaagacggagaggaataattgagaagcgccgacgagaccggatcaataacagtttgtctgagctgagaaggctggtacccagtgcttttgagaagcagggatctgctaagctagaaaaagccgagatcctgcagatgaccgtggatcacctgaaaatgctgcatacggcaggagggaaaggttactttgacgcgcacgcccttgctatggactatcggagtttgggatttcgggaatgcctggcagaagttgcgcgttatctgagcatcattgaaggactagatgcctctgacccgcttcgagttcgactggtttcgcatctcaacaactacgcttcccagcgggaagccgcgagcggcgcccacgcgggcctcggacacattccctgggggaccgtcttcggacatcacccgcacatcgcgcacccgctgttgctgccccagaacggccacgggaacgcgggcaccacggcctcacccacggaaccgcaccaccagggcaggctgggctcggcacatccggaggcgcctgctttgcgagcgccccctagcggcagcctcggaccggtgctccctgtggtcacctccgcctccaaactgtcgccgcctctgctctcctcagtggcctccctgtcggccttccccttctctttcggctccttccacttactgtctcccaatgcactgagcccttcagcacccacgcaggctgcaaaccttggcaagccctatagaccttgggggacggagatcggagctttttaa

SEQ ID NO: 19 - DNA, 1014 bp, Homo sapiens
atgaagcgcccctgcgaggagacgacctccgagagcgacatggacgagaccatcgacgtggggagcgagaacaattactcggggcaaagtactagctctgtgattagattgaattctccaacaacaacatctcagattatggcaagaaagaaaaggagagggattatagagaaaaggcgtcgggatcggataaataacagtttatctgagttgagaagacttgtgccaactgcttttgaaaaacaaggatctgcaaagttagaaaaagctgaaatattgcaaatgacagtggatcatttgaagatgcttcaggcaacagggggtaaaggctactttgacgcacacgctcttgccatggacttcatgagcataggattccgagagtgcctaacagaagttgcgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcggccacagccatcagcccgcccttgtcagtatcagccacgtccagtcctcagcagaccagcagtggaacaaacaataaaccttaccgaccctgggggacagaagttggagctttttaa

SEQ ID NO: 20 - DNA, 987 bp, Homo sapiens
atgaagcgacccaaggagccgagcggctccgacggggagtccgacggacccatcgacgtgggccaagagggccagctgagccagatggccaggccgctgtccacccccagctcttcgcagatgcaagccaggaagaaacgcagagggatcatagagaaacggcgtcgagaccgcatcaacagtagcctttctgaattgcgacgcttggtccccactgcctttgagaaacagggctcttccaagctggagaaagccgaggtcttgcagatgacggtggatcacttgaaaatgctccatgccactggtgggacaggattctttgatgcccgagccctggcagttgacttccggagcattggttttcgggagtgcctcactgaggtcatcaggtacctgggggtccttgaagggcccagcagccgtgcagaccccgtccggattcgccttctctcccacctcaacagctacgcagccgagatggagccttcgcccacgcccactggccctttggccttccctgcctggccctggtctttcttccatagctgtccagggctgccagccctgagcaaccagctcgccatcctgggaagagtgcccagccctgtcctccccggtgtctcctctcctgcttaccccatcccagccctccgaaccgctccccttcgcagagccacaggcatcatcctgccagcccggaggaatgtgctgcccagtcgaggggcatcttccacccggagggcccgccccctagagaggccagcgacccctgtgcctgtcgcccccagcagcagggctgccaggagcagccacatcgctcccctcctgcagtcttcctccccaacaccccctggtcctacagggtcggctgcttacgtggctgttcccacccccaactcatcctccccagggccagctgggaggccagcgggagccatgctctaccactcctgggtctctgaaatcactgaaatcggggctttctga

SEQ ID NO: 21 - DNA, 1331 bp, Homo sapiens
cggccgcgtcgacgggaaagagccgctagagcagaccgcgccgccgccggagccgcgcctgcccaggcccggggagggaggaggcgggcgtcagggtgctgcgccccgctcggcgtccgagcttccggccgggctgtgccccgcgcggtcttcgccgggatgaagcgcccctgcgaggagacgacctccgagagcgacatggacgagaccatcgacgtggggagcgagaacaattactcggggcaaagtactagctctgtgattagattgaattctccaacaacaacatctcagattatggcaagaaagaaaaggagagggattatagagaaaaggcgtcgggatcggataaataacagtttatctgagttgagaagacttgtgccaactgcttttgaaaaacaaggatctgcaaagttagaaaaagctgaaatattgcaaatgacagtggatcatttgaagatgcttcaggcaacagggggtaaaggctactttgacgcacacgctcttgccatggacttcatgagcataggattccgagagtgcctaacagaagttgcgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcggccacagccatcagcccgcccttgtcagtatcagccacgtccagtcctcagcagaccagcagtggaacaaacaataaaccttaccgaccctgggggacagaagttggagctttttaaatttttcttgaacttcttgcaatagtaactgaatgtcctccatttcagagtcagcttaaaacctctgcaccctgaaggtagccatacagatgccgacagatccacaaaggaacaataaagctatttgagacacaaaaagtcgacgcggccgcgaattc

SEQ ID NO: 22 - DNA, 2234 bp, Homo sapiens
gaattcgcggccgcgtcgaccgagagcggagaggcgccgctgtagttaactcctccctgcccgccgcgccgaccctccccaggaacccccagggagccagcatgaagcgagctcaccccgagtacagctcctcggacagcgagctggacgagaccatcgaggtggagaaggagagtgcggacgagaatggaaacttgagttcggctctaggttccatgtccccaactacatcttcccagattttggccagaaaaagacggagaggaataattgagaagcgccgacgagaccggatcaataacagtttgtctgagctgagaaggctggtacccagtgcttttgagaagcagggatctgctaagctagaaaaagccgagatcctgcagatgaccgtggatcacctgaaaatgctgcatacggcaggagggaaaggttactttgacgcgcacgcccttgctatggactatcggagtttgggatttcgggaatgcctggcagaagttgcgcgttatctgagcatcattgaaggactagatgcctctgacccgcttcgagttcgactggtttcgcatctcaacaactacgcttcccagcgggaagccgcgagcggcgcccacgcgggcctcggacacattccctgggggaccgtcttcggacatcacccgcacatcgcgcacccgctgttgctgccccagaacggccacgggaacgcgggcaccacggcctcacccacggaaccgcaccaccagggcaggctgggctcggcacatccggaggcgcctgctttgcgagcgccccctagcggcagcctcggaccggtgctccctgtggtcacctccgcctccaaactgtcgccgcctctgctctcctcagtggcctccctgtcggccttccccttctctttcggctccttccacttactgtctcccaatgcactgagcccttcagcacccacgcaggctgcaaaccttggcaagccctatagaccttgggggacggagatcggagctttttaaagaactgatgtagaatgagggaggggaaagtttaaaatccagctgggctggactgttgccaacatcaccttaaagtcgtcagtaaaagtaaaaaggaaaaaggtacactttcagataattttttttttaaagactaaaggtttgttggtttacttttttcttttttaatgtttttttcatcatgtcatgtattagcagtttttaaaaaactagttgttaaattttgttcaagacattaaattgaaatagtgagtataagccaacactttgtgataggtttgtactgtgcctaatttactttgtaaaccagaatgattccgtttttgcctcaaaatttggggaatcttaacatttagtatttttggtctgtttttctccttgtatagttatggtctgtttttagaattaattttccaaaccactatgcttaatgttaacatgattctgtttgttaatattttgacagattaaggtgttgtataaataatattcttttggggggaggggaactatattgaattttatatttctgagcaaagcgttgacaaatcagatgatcagctttatccaagaaagaagactagtaaattgtctgcctcctatagcagaaaggtgaatgtacaaactgttggtggccctgaatccatctgaccagctgctggtatctgccaggactggcagttctgatttagttaggagagagccgctgataggttaggtctcatttggagtgttggtggaaaggaaactgaaggtaattgaatagaatacgcctgcatttaccagccccagcaacacaaagaatttttaatcacacggatctcaaattcacaaatgttaacatggataagtgatcatggtgtgcgagtggtcaattgagtagtacagtggaacctgttaaatgcataacctaattttcctgggactgccatattttcttttaactggaaatttttatgtgagttttccttttggtgcatggaactgtggttgccaaggtatttaaaagggctttcctgcctccttctctttgatttatttaatttgatttgggctataaaatatcatttttcaggtttattcttttagcaggtgtagttaaacgacctccactgaactgggtttgacctctgttgtactgatgtgttgtgactaaataaaaaagaaagaacaaagaaaaaaaaaaaaaaaaaaaaaaaaaaaaaaaaaaaaaaaaaa

SEQ ID NO: 23 - DNA, 1596 bp, Homo sapiens
agggtgctgcgccccgctcggcgtccgagcttccggccgggctgtgccccgcgcggtcttcgccgggatgaagcgcccctgcgaggagacgacctccgagagcgacatggacgagaccatcgacgtggggagcgagaacaattactcggggcaaagtactagctctgtgattagattgaattctccaacaacaacatctcagattatggcaagaaagaaaaggagagggattatagagaaaaggcgtcgggatcggataaataacagtttatctgagttgagaagacttgtgccaactgcttttgaaaaacaaggatctgcaaagttagaaaaagctgaaatattgcaaatgacagtggatcatttgaagatgcttcaggcaacagggggtaaaggctactttgacgcacacgctcttgccatggacttcatgagcataggattccgagagtgcctaacagaagttgcgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcggccacagccatcagcccgcccttgtcagtatcagccacgtccagtcctcagcagaccagcagtggaacaaacaataaaccttaccgaccctgggggacagaagttggagctttttaaatttttcttgaacttcttgcaatagtaactgaatgtcctccatttcagagtcagcttaaaacctctgcaccctgaaggtagccatacagatgccgacagatccacaaaggaacaataaagctatttgagacacaaacctcacgagtggaaatgtggtattctcttttttttctctcccttttttgtttggttcaaggcagctcggtaactgacatcagcaacttttgaaaacttcacacttgttaccatttagaagtttcctggaaaatatatggaccgtaccatccagcagtgcatcagtatgtctgaattggggaagtaaaatgccctgactgaattctcttgagactagatgggacatacatatatagagagagagtgagagagtcgtgtttcgtaagtgcctgagcttaggaagttttcttctggatatataacattgcacaagggaagacgagtgtggaggataggttaagaaaggaaagggacagaagtcttgcaataggctgcagaca

SEQ ID NO: 24 - DNA, 1286 bp, Homo sapiens
ctccggtccccgccgctccccgtccccgctgctcctagcccctgccgcgtccccggcggagcgggcatggcgccacccgcggcgcctggccgggaccgtgtgggccgtgaggatgaggacggctgggagacgcgaggggaccgcaaggcccggaagcccctggtggagaagaagcggcgcgcgcggatcaacgagagcctgcaggagctgcggctgctgctggcgggcgccgaggccaagctggagaacgccgaagtgctggagctgacggtgcggcgggtccagggtgtgctgcggggccgggcgcgcgagcgcgagcagctgcaggcggaagcgagcgagcgcttcgctgccggctacatccagtgcatgcacgaggtgcacacgttcgtgtccacgtgccaggccatcgacgctaccgtcgctgccgagctcctgaaccatctgctcgagtccatgccgctgcgtgagggcagcagcttccaggatctgctgggggacgccctggcggggccacctagagcccctggacggagtggctggcctgcggggggcgctccgggatccccaatacccagccccccgggtcctggggacgacctgtgctccgacctggaggaggcccctgaggctgaactgagtcaggctcctgctgaggggcccgacttggtgcccgcagccctgggcagcctgaccacagcccaaattgcccggagtgtctggaggccttggtgaccaatgccagccagagtcctgcgggggtgggcccggccctccctggatctcctccctcctcccaggggttcagatgtggtggggtagggccctggaagtctcccaggtcttccctccctcctctgatggatggcttgcagggcagcccctggtaaccagcccagtcaggccccagccccgtttcttaagaaacttttagggaccctgcagctctggagtgggtggagggagggagctacgggcaggaggaagaattttgtagagctgccagcgctctcccaggttcacccacccaggcttcaccagccctgtgcgggctctgggggcagaggtggcagaaatggtgctgggcactagtgttccaggcagccctgggctaaacaaaagcttgaacttgccacttcagcggggagatgagaggcaggtgcactgagctgcactgcccagagctgtgatgctctgtacatcttgtttgtagcacacttgagtttgtgtattccattgacatcaaatgtgacaattttactaaataaagaattttggagttagtt

SEQ ID NO: 25 - DNA, 678 bp, Homo sapiens
atggtcacccgggatcgagctgagaatagggacggccccaagatgctcaagccgcttgtggagaagcggcgccgggaccgcatcaaccgcagcctggaagagctgaggctgctgctgctggagcggacccgggaccagaacctccggaacccgaagctggagaaagcggagatattggagttcgccgtgggctacttgagggagcgaagccgggtggagcccccgggggttccccggtccccagtccaggacgccaaggcgctcgccagctgctacttgtccggtttccgcgagtgcctgcttcgcttggcggccatcgcgcacgacgccagcccgaccgcccgcgcccagctcttctccgcgctgcacggctatctgcgccccaaaccgccccggcccaagccggtagatccgaggcctccagcgccgcgcccatccctggaccccgccgcaccggcccttggccctgcgctgcaccagcgccccccagtgcaccagggccaccctagcccgcgctgcgcatggtccccatccctctgctccccgcgcgccggggattctggcgcgccggcgcccctcaccggactgctgccgccgccaccgccgcctcacagacaagacggggcgcccaaggccccgctgcccccgccgcccgctttctggagaccttggccctga

SEQ ID NO: 26 - DNA, 1307 bp, Homo sapiens
cagcggtgcgagctccaggcccatgcactgaggaggcggaaacaaggggagcccccagagctccatcaagccccctccaaaggctcccctacccggtccacgccccccaccccccctccccgcctcctcccaattgtgcatttttgcagccggaggcggctccgagatggggctgtgagcttcgcccggggagggggaaagagcagcgaggagtgaagcgggggggtggggtgaagggtttggatttcggggcagggggcgcacccccgtcagcaggccctccccaaggggctcggaactctacctcttcacccacgcccctggtgcgctttgccgaaggaaagaataagaacagagaaggaggagggggaaaggaggaaaagggggaccccccaactggggggggtgaaggagagaagtagcaggaccagaggggaaggggctgctgcttgcatcagcccacaccatgctgaccccgccgttgctcctgctgctgcccctgctctcagctctggtcgcggcggctatcgacgcccctaagacttgcagccccaagcagtttgcctgcagagatcaaataacctgtatctcaaagggctggcggtgcgacggtgagagggactgcccagacggatctgacgaggcccctgagatttgtccacagagtaaggcccagcgatgccagccaaacgagcataactgcctgggtactgagctgtgtgttcccatgtcccgcctctgcaatggggtccaggactgcatggacggctcagatgaggggccccactgccgagagctccaaggcaactgctctcgcctgggctgccagcaccattgtgtccccacactcgatgggcccacctgctactgcaacagcagctttcagcttcaggcagatggcaagacctgcaaagattttgatgagtgctcagtgtacggcacctgcagccagctatgcaccaacacagacggctccttcatatgtggctgtgttgaaggatacctcctgcagccggataaccgctcctgcaaggccaagaacgagccagtagaccggccccctgtgctgttgatagccaactcccagaacatcttggccacgtacctgagtggggcccaggtgtctaccatcacacctacgagcacgcggcagaccacagccatggacttcagctatgccaacgagaccgtatgctgggtgcatgttggggacagtgctgctcagacgcagctcaagtgtgcccgcatgcctggcctaaagggcttcgtggatgagcacaccatcaacatctccctcagtctgcacc

SEQ ID NO: 27 - DNA, 1307 bp, Homo sapiens; identical to SEQ ID NO: 26

SEQ ID NO: 28 - DNA, 1307 bp, Homo sapiens; identical to SEQ ID NO: 26

SEQ ID NO: 29 - DNA, 4700 bp, Homo sapiens
caggcatgaacagtttcctcatcttcgccaggaggatagacattcgcatggtctccctggacatcccttattttgctgatgtggtggtaccaatcaacattaccatgaagaacaccattgccattggagtagacccccaggaaggaaaggtgtactggtctgacagcacactgcacaggatcagtcgtgccaatctggatggctcacagcatgaggacatcatcaccacagggctacagaccacagatgggctcgcggttgatgccattggccggaaagtatactggacagacacgggaacaaaccggattgaagtgggcaacctggacgggtccatgcggaaagtgttggtgtggcagaaccttgacagtccccgggccatcgtactgtaccatgagatggggtttatgtactggacagactggggggagaatgccaagttagagcggtccggaatggatggctcagaccgcgcggtgctcatcaacaacaacctaggatggcccaatggactgactgtggacaaggccagctcccaactgctatgggccgatgcccacaccgagcgaattgaggctgctgacctgaatggtgccaatcggcatacattggtgtcaccggtgcagcacccatatggcctcaccctgctcgactcctatatctactggactgactggcagactcggagcatccaccgtgctgacaagggtactggcagcaatgtcatcctcgtgaggtccaacctgccaggcctcatggacatgcaggctgtggaccgggcacagccactaggttttaacaagtgcggctcgagaaatggcggctgctcccacctctgcttgcctcggccttctggcttctcctgtgcctgccccactggcatccagctgaagggagatgggaagacctgtgatccctctcctgagacctacctgctcttctccagccgtggctccatccggcgtatctcactggacaccagtgaccacaccgatgtgcatgtccctgttcctgagctcaacaatgtcatctccctggactatgacagcgtggatggaaaggtctattacacagatgtgttcctggatgttatcaggcgagcagacctgaacggcagcaacatggagacagtgatcgggcgagggctgaagaccactgacgggctggcagtggactgggtggccaggaacctgtactggacagacacaggtcgaaataccattgaggcgtccaggctggatggttcctgccgcaaagtactgatcaacaatagcctggatgagccccgggccattgctgttttccccaggaaggggtacctcttctggacagactggggccacattgccaagatcgaacgggcaaacttggatggttctgagcggaaggtcctcatcaacacagacctgggttggcccaatggccttaccctggactatgatacccgcaggatctactgggtggatgcgcatctggaccggatcgagagtgctgacctcaatgggaaactgcggcaggtcttggtcagccatgtgtcccacccctttgccctcacacagcaagacaggtggatctactggacagactggcagaccaagtcaatccagcgtgttgacaaatactcaggccggaacaaggagacagtgctggcaaatgtggaaggactcatggatatcatcgtggtttcccctcagcggcagacagggaccaatgcctgtggtgtgaacaatggtggctgcacccacctctgctttgccagagcctcggacttcgtatgtgcctgtcctgacgaacctgatagccggccctgctcccttgtgcctggcctggtaccaccagctcctagggctactggcatgagtgaaaagagcccagtgctacccaacacaccacctaccaccttgtattcttcaaccacccggacccgcacgtctctggaggaggtggaaggaagatgctctgaaagggatgccaggctgggcctctgtgcacgttccaatgacgctgttcctgctgctccaggggaaggacttcatatcagctacgccattggtggactcctcagtattctgctgattttggtggtgattgcagctttgatgctgtacagacacaaaaaatccaagttcactgatcctggaatggggaacctcacctacagcaacccctcctaccgaacatccacacaggaagtgaagattgaagcaatccccaaaccagccatgtacaaccagctgtgctataagaaagagggagggcctgaccataactacaccaaggagaagatcaagatcgtagagggaatctgcctcctgtctggggatgatgctgagtgggatgacctcaagcaactgcgaagctcacgggggggcctcctccgggatcatgtatgcatgaagacagacacggtgtccatccaggccagctctggctccctggatgacacagagacggagcagctgttacaggaagagcagtctgagtgtagcagcgtccatactgcagccactccagaaagacgaggctctctgccagacacgggctggaaacatgaacgcaagctctcctcagagagccaggtctaaatgcccacattctcttccctgcctgcctgttccttctcctttatggacgtctagtccttgtgctcgcttacaccgcaggccccgcttctgtgtgcttgtcctcctcctcctcccaccccataactgttcctaagccttcaccggagctgtttaccacgtgagtccataactacctgtgcacaagaaatgatggcacatcacgagaatttagacctggattttaccatgaacctcacatcttgtactccatcctgggccccctgaaactgcttattcgtgattcctcaccagcgtagagctccacctcccctttccccagtaccctcagtgcctgcttctcagtgctgatgcagctgatgacccaggactgcgctctgccccatcacagccagcatgactgcttctctgagagaacttgcccatcaggggctgggacatgggggtgtgggtaaagacagggatgaaggatagaggctgagagaagaaggaagaatcagcccagcaggtatgggcatctgggaaacctccagcctcaagtgtgttagtaacatgaaaaagctttggggggtagttggatctgggtgtctggtccattgctggcagtggacattattcttgccctaagagacactgccttttcagcagcagatactggtgagatgggggtggctcaggctgttcttcctcctcctagaatgtctggagctgtttctacattcagataactgggtcccctatcacaaggctactggctaataggaattccctcctggtgccaccactggccagtacctttcctaagtctttgctcaaattaaccaggttgtgagccagtggcttgagtgaatgttaggccttgggggctgagtctctgaaaagtctaagaagctctgcctagaccaaatatggtatacctcctgacccctctctccctcatgtcctgggattctggggaagagacctagaaacaagctttcaaagaaaaaccagaagttgtcataaatggtcagaaagaacgatcaggttggagacttgggaaacccagggcctaaagagaagtatccatgagggtcaaacttcctgttgaacttcctatgttctttctcaagtgctcagggatctaagttagtggacagcaagcctgtggctacggggtggtgatgttcctcttccagctgtcccctcagctaaggggcttagtttccatgtgggatgccatcacttggttcatgctcattcacacaaagggcacgtgtctcagcctggtatcagggaaattgagacttatttttgccctaaaacgtctccctagctgttcttcgtggggtttttttgtttgtttttttgcctaatttgctttttctgaccaagccttgtggcaccagcaatctccaaagtcctgtggtgggagggctgaataaataaaaatacaaagaggtgggtaaggagtaggaaggtagagagcaccactgatgaggccctcctagcccatggcagacccagacctcttctcccccaggaattagaagtggcaggagagaacaacaggggctgggaatggaggggagaatttctaggggaagtttcctgagttgaaacttctcctgtggttactggtattgagaaatcagctaccaaagtgaaaaaggacaagatcaattcttttctagtcagttctaagactgctagagagagataccaggcccttagccttgctctcagtagcgtcagccccagttctgagcctccccacattacacttaacaagcagtaaaggagtgagcactttgggtccttagactcacgtctggggaggaagagcaagtagaaaagtggcattttcttgattggaaagggggaaggatcttattgcacttgggctgttcagaatgtagaaaggacatatttgaggaagtatctatttgagcactgatttactctgtaaaaagcaaaatctctctgtcctaaactaatggaagcgattctcccatgctcatgtgtaatggttttaacgttactcactggagagattggactttctggagttatttaaccactatgttcagtattttaggactttatgataatttaatataaatttagcttttcttaatc

SEQ ID NO: 30 - DNA, 5100 bp, Homo sapiens
atggagcccgagtgagcgcggcgcgggcccgtccggccgccggacaacatggaggcagcgccgcccgggccgccgtggccgctgctgctgctgctgctgctgctgctggcgctgtgcggctgcccggcccccgccgcggcctcgccgctcctgctatttgccaaccgccgggacgtacggctggtggacgccggcggagtcaagctggagtccaccatcgtggtcagcggcctggaggatgcggccgcagtggacttccagttttccaagggagccgtgtactggacagacgtgagcgaggaggccatcaagcagacctacctgaaccagacgggggccgccgtgcagaacgtggtcatctccggcctggtctctcccgacggcctcgcctgcgactgggtgggcaagaagctgtactggacggactcagagaccaaccgcatcgaggtggccaacctcaatggcacatcccggaaggtgctcttctggcaggaccttgaccagccgagggccatcgccttggaccccgctcacgggtacatgtactggacagactggggtgagacgccccggattgagcgggcagggatggatggcagcacccggaagatcattgtggactcggacatttactggcccaatggactgaccatcgacctggaggagcagaagctctactgggctgacgccaagctcagcttcatccaccgtgccaacctggacggctcgttccggcagaaggtggtggagggcagcctgacgcaccccttcgccctgacgctctccggggacactctgtactggacagactggcagacccgctccatccatgcctgcaacaagcgcactggggggaagaggaaggagatcctgagtgccctctactcacccatggacatccaggtgctgagccaggagcggcagcctttcttccacactcgctgtgaggaggacaatggcggctgctcccacctgtgcctgctgtccccaagcgagcctttctacacatgcgcctgccccacgggtgtgcagctgcaggacaacggcaggacgtgtaaggcaggagccgaggaggtgctgctgctggcccggcggacggacctacggaggatctcgctggacacgccggactttaccgacatcgtgctgcaggtggacgacatccggcacgccattgccatcgactacgacccgctagagggctatgtctactggacagatgacgaggtgcgggccatccgcagggcgtacctggacgggtctggggcgcagacgctggtcaacaccgagatcaacgaccccgatggcatcgcggtcgactgggtggcccgaaacctctactggaccgacacgggcacggaccgcatcgaggtgacgcgcctcaacggcacctcccgcaagatcctggtgtcggaggacctggacgagccccgagccatcgcactgcaccccgtgatgggcctcatgtactggacagactggggagagaaccctaaaatcgagtgtgccaacttggatgggcaggagcggcgtgtgctggtcaatgcctccctcgggtggcccaacggcctggccctggacctgcaggaggggaagctctactggggagacgccaagacagacaagatcgaggtgatcaatgttgatgggacgaagaggcggaccctcctggaggacaagctcccgcacattttcgggttcacgctgctgggggacttcatctactggactgactggcagcgccgcagcatcgagcgggtgcacaaggtcaaggccagccgggacgtcatcattgaccagctgcccgacctgatggggctcaaagctgtgaatgtggccaaggtcgtcggaaccaacccgtgtgcggacaggaacggggggtgcagccacctgtgcttcttcacaccccacgcaacccggtgtggctgccccatcggcctggagctgctgagtgacatgaagacctgcatcgtgcctgaggccttcttggtcttcaccagcagagccgccatccacaggatctccctcgagaccaataacaacgacgtggccatcccgctcacgggcgtcaaggaggcctcagccctggactttgatgtgtccaacaaccacatctactggacagacgtcagcctgaagaccatcagccgcgccttcatgaacgggagctcggtggagcacgtggtggagtttggccttgactaccccgagggcatggccgttgactggatgggcaagaacctctactgggccgacactgggaccaacagaatcgaagtggcgcggctggacgggcagttccggcaagtcctcgtgtggagggacttggacaacccgaggtcgctggccctggatcccaccaagggctacatctactggaccgagtggggcggcaagccgaggatcgtgcgggccttcatggacgggaccaactgcatgacgctggtggacaaggtgggccgggccaacgacctcaccattgactacgctgaccagcgcctctactggaccgacctggacaccaacatgatcgagtcgtccaacatgctgggtcaggagcgggtcgtgattgccgacgatctcccgcacccgttcggtctgacgcagtacagcgattatatctactggacagactggaatctgcacagcattgagcgggccgacaagactagcggccggaaccgcaccctcatccagggccacctggacttcgtgatggacatcctggtgttccactcctcccgccaggatggcctcaatgactgtatgcacaacaacgggcagtgtgggcagctgtgccttgccatccccggcggccaccgctgcggctgcgcctcacactacaccctggaccccagcagccgcaactgcagcccgcccaccaccttcttgctgttcagccagaaatctgccatcagtcggatgatcccggacgaccagcacagcccggatctcatcctgcccctgcatggactgaggaacgtcaaagccatcgactatgacccactggacaagttcatctactgggtggatgggcgccagaacatcaagcgagccaaggacgacgggacccagccctttgttttgacctctctgagccaaggccaaaacccagacaggcagccccacgacctcagcatcgacatctacagccggacactgttctggacgtgcgaggccaccaataccatcaacgtccacaggctgagcggggaagccatgggggtggtgctgcgtggggaccgcgacaagcccagggccatcgtcgtcaacgcggagcgagggtacctgtacttcaccaacatgcaggaccgggcagccaagatcgaacgcgcagccctggacggcaccgagcgcgaggtcctcttcaccaccggcctcatccgccctgtggccctggtggtagacaacacactgggcaagctgttctgggtggacgcggacctgaagcgcattgagagctgtgacctgtcaggggccaaccgcctgaccctggaggacgccaacatcgtgcagcctctgggcctgaccatccttggcaagcatctctactggatcgaccgccagcagcagatgatcgagcgtgtggagaagaccaccggggacaagcggactcgcatccagggccgtgtcgcccacctcactggcatccatgcagtggaggaagtcagcctggaggagttctcagcccacccatgtgcccgtgacaatggtggctgctcccacatctgtattgccaagggtgatgggacaccacggtgctcatgcccagtccacctcgtgctcctgcagaacctgctgacctgtggagagccgcccacctgctccccggaccagtttgcatgtgccacaggggagatcgactgtatccccggggcctggcgctgtgacggctttcccgagtgcgatgaccagagcgacgaggagggctgccccgtgtgctccgccgcccagttcccctgcgcgcggggtcagtgtgtggacctgcgcctgcgctgcgacggcgaggcagactgtcaggaccgctcagacgaggcggactgtgacgccatctgcctgcccaaccagttccggtgtgcgagcggccagtgtgtcctcatcaaacagcagtgcgactccttccccgactgtatcgacggctccgacgagctcatgtgtgaaatcaccaagccgccctcagacgacagcccggcccacagcagtgccatcgggcccgtcattggcatcatcctctctctcttcgtcatgggtggtgtctattttgtgtgccagcgcgtggtgtgccagcgctatgcgggggccaacgggcccttcccgcacgagtatgtcagcgggaccccgcacgtgcccctcaatttcatagccccgggcggttcccagcatggccccttcacaggcatcgcatgcggaaagtccatgatgagctccgtgagcctgatggggggccggggcggggtgcccctctacgaccggaaccacgtcacaggggcctcgtccagcagctcgtccagcacgaaggccacgctgtacccgccgatcctgaacccgccgccctccccggccacggacccctccctgtacaacatggacatgttctactcttcaaacattccggccactgtgagaccgtacaggccctacatcattcgaggaatggcgcccccgacgacgccctgcagcaccgacgtgtgtgacagcgactacagcgccagccgctggaaggccagcaagtactacctggatttgaactcggactcagacccctatccacccccacccacgccccacagccagtacctgtcggcggaggacagctgcccgccctcgcccgccaccgagaggagctacttccatctcttcccgccccctccgtccccctgcacggactcatcctgacctcggccgggccactctggcttctctgtgcccctgtaaatagttttaaatatgaacaaagaaaaaaatatattttatgatttaaaaaataaatataattgggattttaaaaacatgagaaatgtgaactgtgatggggtgggcagggctgggagaactttgtacagtggaacaaatatttataaacttaattttgtaaaacag

SEQ ID NO: 31 - DNA, 5301 bp, Homo sapiens
gcggccgccccggctcctcgcctcccccacttctggccacccctcgccggtgagagaagagaacgcgagaagggaagatgggggccgtcctgaggagcctcctggcctgcagcttctgtgtgctcctgagagcggcccctttgttgctttatgcaaacagacgggacttgcgattggttgatgctacaaatggcaaagagaatgctacgattgtagttggaggcttggaggatgcagctgcggtggactttgtgtttagtcatggcttgatatactggagtgatgtcagcgaagaagccattaaacgaacagaatttaacaaaactgagagtgtgcagaatgttgttgtttctggattattgtcccccgatgggctggcatgtgattggcttggagaaaaattgtactggacagattctgaaactaatcggattgaagtttctaatttagatggatctttacgaaaagttttattttggcaagagttggatcaacccagagctattgccttagatccttcaagtgggttcatgtactggacagactggggagaagtgccaaagatagaacgtgctggaatggatggttcaagtcgcttcattataataaacagtgaaatttactggccaaatggactgactttggattatgaagaacaaaagctttattgggcagatgcaaaacttaatttcatccacaaatcaaatctggatggaacaaatcggcaggcagtggttaaaggttcccttccacatccttttgccttgacgttatttgaggacatattgtactggactgactggagcacacactccattttggcttgcaacaagtatactggtgagggtctgcgtgaaatccattctgacatcttctctcccatggatatacatgccttcagccaacagaggcagccaaatgccacaaatccatgtggaattgacaatgggggttgttcccatttgtgtttgatgtctccagtcaagcctttttatcagtgtgcttgccccactggggtcaaactcctggagaatggaaaaacctgcaaagatggtgccacagaattattgcttttagctcgaaggacagacttgagacgcatttctttggatacaccagattttacagacattgttctgcagttagaagacatccgtcatgccattgccatagattacgatcctgtggaaggctacatctactggactgatgatgaagtgagggccatacgccgttcatttatagatggatctggcagtcagtttgtggtcactgctcaaattgcccatcctgatggtattgctgtggactgggttgcacgaaatctttattggacagacactggcactgatcgaatagaagtgacaaggctcaatgggaccatgaggaagatcttgatttcagaggacttagaggaaccccgggctattgtgttagatcccatggttgggtacatgtattggactgactggggagaaattccgaaaattgagcgagcagctctggatggttctgaccgtgtagtattggttaacacttctcttggttggccaaatggtttagccttggattatgatgaaggcaaaatatactggggagatgccaaaacagacaagattgaggttatgaatactgatggcactgggagacgagtactagtggaagacaaaattcctcacatatttggatttactttgttgggtgactatgtttactggactgactggcagaggcgtagcattgaaagagttcataaacgaagtgcagagagggaagtgatcatagatcagctgcctgacctcatgggcctaaaggctacaaatgttcatcgagtgattggttccaacccctgtgctgaggaaaacgggggatgtagccatctctgcctctatagacctcagggccttcgctgtgcttgccctattggctttgaactcatcagtgacatgaagacctgcattgtcccagaggctttccttttgttttcacggagagcagatatcagacgaatttctctggaaacaaacaataataatgtggctattccactcactggtgtcaaagaagcttctgctttggattttgatgtgacagacaaccgaatttattggactgatatatcactcaagaccatcagcagagcctttatgaatggcagtgcactggaacatgtggtagaattcggcttagattatccagaaggcatggcagtagactggcttgggaagaacttgtactgggcagacacaggaacgaatcgaattgaggtgtcaaagttggatgggcagcaccgacaagttttggtgtggaaagacctagatagtcccagagctctcgcgttggaccctgccgaaggatttatgtattggactgaatggggtggaaaacctaagatagacagagctgcaatggatggaagtgaacgtactaccttagttccaaatgtggggcgggcaaacggcctaactattgattatgctaaaaggaggctttattggacagacctggacaccaacttaatagaatcttcaaatatgcttgggctcaaccgtgaagttatagcagatgacttgcctcatccttttggcttaactcagtaccaagattatatctactggacggactggagccgacgcagcattgagcgtgccaacaaaaccagtggccaaaaccgcaccatcattcagggccatttggattatgtgatggacatcctcgtctttcactcatctcgacagtcagggtggaatgaatgtgcttccagcaatgggcactgctcccacctctgcttggctgtgccagttgggggttttgtttgtggatgccctgcccactactctcttaatgctgacaacaggacttgtagtgctcctacgactttcctgctcttcagtcaaaagagtgccatcaaccgcatggtgattgatgaacaacagagccccgacatcatccttcccatccacagccttcggaatgtccgggccattgactatgacccactggacaagcaactctattggattgactcacgacaaaacatgatccgaaaggcacaagaagatggcagccagggctttactgtggttgtgagctcagttccgagtcagaacctggaaatacaaccctatgacctcagcattgatatttacagccgctacatctactggacttgtgaggctaccaatgtcattaatgtgacaagattagatgggagatcagttggagtggtgctgaaaggcgagcaggacagacctcgagccattgtggtaaacccagagaaagggtatatgtattttaccaatcttcaggaaaggtctcctaaaattgaacgggctgctttggatgggacagaacgggaggtcctctttttcagtggcttaagtaaaccaattgctttagcccttgatagcaggctgggcaagctcttttgggctgattcagatctccggcgaattgaaagcagtgatctctcaggtgctaaccggatagtattagaagactccaatatcttgcagcctgtgggacttactgtgtttgaaaactggctctattggattgataaacagcagcaaatgattgaaaaaattgacatgacaggtcgagagggtagaaccaaagtccaagctcgaattgcccagcttagtgacattcatgcagtaaaggagctgaaccttcaagaatacagacagcacccttgtgctcaggataatggtggctgttcacatatttgtcttgtaaagggggatggtactacaaggtgttcttgccccatgcacctggttctacttcaagatgagctatcatgtggagaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcctgaggaggggccctcctcctctgactgcctccaacgtaaaaatgtaaatataaatttggttgagatctggagggggggagggagctattagagaaggatgaggcagaccatgtacagttaaaattataaaatggggtagggaatactggagatatttgtacagaagaaaaggatatttatatattttcttaaaacagcagatttgctgcttgtgccataaaagtttgtataaaaaaaatttgtactaaaagttttatttttgcaaactaaatacacaaagcatgccttaaacccagtgaagcaactgagtacaaaggaaacaggaataataaaggcatcactgaccaggaatatctgggctttattgataccaaaaaaaaaaaaa

SEQ ID NO: 32 - DNA, 3941 bp, Homo sapiens
tgctgctgctgctgctgctgctgctgctgcagctccagcatcttgcggcggcagcggctgatccgctgctcggcggccaagggccggccaaggattgcgaaaaggaccaattccagtgccggaacgagcgctgcatcccctctgtgtggagatgcgacgaggacgatgactgcttagaccacagcgacgaggacgactgccccaagaagacctgtgcagacagtgacttcacctgtgacaacggccactgcatccacgaacggtggaagtgtgacggcgaggaggagtgtcctgatggctccgatgagtccgaggccacttgcaccaagcaggtgtgtcctgcagagaagctgagctgtggacccaccagccacaagtgtgtacctgcctcgtggcgctgcgacggggagaaggactgcgagggtggagccggatgaggccggctgtgctacctcactgggcacctgccgtggggacgagttccagtgtggggatgggacatgtgtccttgcaatcaagcactgcaaccaggagcaggactgtccagatgggagtgatgaagctggctgcctacaggggctgaacgagtgtctgcacaacaatggcggctgctcacacatctgcactgacctcaagattggctttgaatgcacgtgcccagcaggcttccagctcctggaccagaagacctgtggcgacattgatgagtgcaaggacccagatgcctgcagccagatctgtgtcaattacaagggctattttaagtgtgagtgctaccctggctacgagatggacctactgaccaagaactgcaaggctgctggtggcaagagcccatccctaatcttcaccaaccggcacgaggtgcggaggatcgacctggtgaagcggaactattcacgcctcatccccatgctcaagaatgtcgtggcactagatgtggaagttgccaccaatcgcatctactggtgtgacctctcctaccgtaagatctatagcgcctacatggacaaggccagtgacccgaaagagcaggaggtcctcattgacgagcagttgcactctccagagggcctggcagtggactgggtccacaagcacatctactggactgactcgggcaataagaccatctcagtggccacagttgatggtggccgccgacgcactctcttcagccgtaacctcagtgaaccccgggccatcgctgttgaccccctgcgagggttcatgtattggtctgactggggggaccaggccaagattgagaaatctgggctcaacggtgtggaccggcaaacactggtgtcagacaatattgaatggcccaacggaatcaccctggatctgctgagccagcgcttgtactgggtagactccaagctacaccaactgtccagcattgacttcagtggaggcaacagaaagacgctgatctcctccactgacttcctgagccacccttttgggatagctgtgtttgaggacaaggtgttctggacagacctggagaacgaggccattttcagtgcaaatcggctcaatggcctggaaatctccatcctggctgagaacctcaacaacccacatgacattgtcatcttccatgagctgaagcagccaagagctccagatgcctgtgagctgagtgtccagcctaatggaggctgtgaatacctgtgccttcctgctcctcagatctccagccactctcccaagtacacatgtgcctgtcctgacacaatgtggctgggtccagacatgaagaggtgctaccgagcacctcaatctacctcaactacgacgttagcttctaccatgacgaggacagtacctgccaccacaagagcccccgggaccaccgtccacagatccacctaccagaaccacagcacagagacaccaagcctgacagctgcagtcccaagctcagttagtgtccccagggctcccagcatcagcccgtctaccctaagccctgcaaccagcaaccactcccagcactatgcaaatgaagacagtaagatgggctcaacagtcactgccgctgttatcgggatcatcgtgcccatagtggtgatagccctcctgtgcatgagtggatacctgatctggagaaactggaagcggaagaacaccaaaagcatgaattttgacaacccagtctacaggaaaacaacagaagaagaagacgaagatgagctccatatagggagaactgctcagattggccatgtctatcctgcagcaatcagcagctttgatcgcccactgtgggcagagccctgtcttggggagaccagagaaccggaagacccagcccctgccctcaaggagctttttgtcttgccgggggaaccaaggtcacagctgcaccaactcccgaagaaccctctttccgagctgcctgtcgtcaaatccaagcgagtggcattaagccttgaagatgatggactaccctgaggatgggatcacccccttcgtgcctcatggaattcagtcccatgcactacactctggatggtgtatgactggatgaatgggtttctatatatgggtctgtgtgagtgtatgtgtgtgtgtgattttttttttaaatttatgttgcggaaaggtaaccacaaagttatgatgaactgcaaacatccaaaggatgtgagagtttttctatgtataatgttttatacactttttaactggttgcactacccatgaggaattcgtggaatggctactgctgactaacatgatgcacataaccaaatgggggccaatggcacagtaccttactcatcatttaaaaactatatttacagaagatgtttggttgctgggggggcttttttaggttttggggcatttgttttttgtaaataagatgattatgctttgtggctatccatcaacataagtaaaaaaaaaaaaaaaacacttcaactccctcccccatttagattatttattaacatattttaaaaatcagatgagttctataaataatttagagaagtgagagtatttatttttggcatgtttggcccaccacacagactctgtgtgtgtatgtgtgtgtttatatgtgtatgtgtgtgacagaaaaatctgtagagaagaggcacatctatggctactgttcaaatacataaagataaatttattttcacacagtccacaaggggtatatcttgtagttttcagaaaagcctttggaaatctggatcagaaaatagataccatggtttgtgcaattatgtagtaaaaaaggcaaatcttttcacctctggctattcctgagaccccaggaagtcaggaaaagcctttcagctcacccatggctgctgtgactcctaccagggctttcttggctttggcgaaggtcagtgtacagacattccatggtaccagagtgctcagaaactcaagataggatatgcctcaccctcagctactccttgttttaaagttcagctctttgagtaacttcttcaatttctttcaggacacttgggttgaattcagtaagtttcctctgaagcaccctgaagggtgccatccttacagagctaagtggagacgtttccagatcagcccaagtttactatagagactggcccaggcactgaatgtctaggacatgctgtggatgaagataaagatggtggaataggttttatcacatctcttatttctcttttccccttactctctaccatttcctttatgtggggaaacattttaaggtaataaataggttacttaccatcatatgttcatatagatgaaactaatttttggcttaagtcagaacaactggccaaaattgaagtcatatttgaggggggaaatggcatacgcaatattatattatattggatatttatgttcacacaggaatttggtttactgctttgtaaataaaaggaaaaactccgggtatatgt

SEQ ID NO: 33 - DNA, 1727 bp, Homo sapiens
gcaatggccacctttagccgccaggaatttttccagcagctactgcaaggctgtctcctgcctactgcccagcagggccttgaccagatctggctgctccttgccatctgcctcgcctgccgcctcctctggaggctcgggttgccatcctacctgaagcatgcaagcaccgtggcaggcgggttcttcagcctctaccacttcttccagctgcacatggtttgggtcgtgctgctcagcctcctgtgctacctcgtgctgttcctctgccgacattcctcccatcgaggcgtcttcctatccgtcaccatcctcatctacctactcatgggtgagatgcacatggtagacaccgtgacatggcacaagatgcgaggggcacagatgattgtggccatgaaggcagtgtctctgggcttcgacctggaccggggcgaggtgggtacggtgccctcgccagtggagttcatgggctacctctacttcgtgggcaccatcgtcttcgggccctggatatccttccacagctacctacaaactgtccaaggccgcccactgagctgccggtggctgcagaaggtggcccggagcctggcactggccctgctgtgccttgtgctgtccacttgcgtgggcccctacctcttcccgtacttcatccccctcaacggtgaccgcctccttcgcaacaagaaacgcaaagccaggggcaccatggtaaggtggctgcgagcctacgagagtgctgtctccttccacttcagcaactattttgtgggctttctttccgaggccacggccacgttggcgggggctggctttaccgaggagaaggatcacctggaatgggacctgacggtgtccaagccactgaatgtggagctgcctcggtcaatggtggaagttgtcacaagctggaacctgcccatgtcttattggctaaataactatgttttcaagaatgctctccgcctggggaccttctcggctgtgctggtcacctatgcagccagcgccctcctacatggcttcagtttccacctggctgcggtcctgctgtccctggcttttatcacttacgtggagcatgtcctccggaagcgcctggctcggatcctcagtgcctgtgtcttgtcaaagcggtgcccgccagactgttcgcaccagcatcgcttgggcctgggggtgcgagccttaaacttgctctttggagctctggccatcttccacctggcctacctgggctccctgtttgatgtcgatgtggatgacaccacagaggagcagggctacggcatggcatacactgtccacaagtggtcagagctcagctgggccagtcactgggtcacttttggatgctggatcttctaccgtctcataggctgaggcacatctgtggaccctcataaccctcttaagacccctctcagggtgccactgatgggggatgagggaaggccctctctctactccttgaccccctccatccttgacccccaacacctcaacacacacacacacacacacacacaaaatcacaccattttcatgcctgtcaatccccaccccaccacaaggcaggaagggggtggtgcctgctggggcctagagggggatgcttggggaaacagagaaggggagatccagggcctccccgccttccttcttcctttttatatacaatttgttattgtcaaataaaagtaggaaatattcaataggct

SEQ ID NO: 34 - DNA, 1475 bp, Homo sapiens
cggcacggtttcgtggggacccaggcttgcaaagtgacggtcattttctctttctttctccctcttgagtccttctgagatgatggctctgggcgcagcgggagctacccgggtctttgtcgcgatggtagcggcggctctcggcggccaccctctgctgggagtgagcgccaccttgaactcggttctcaattccaacgctatcaagaacctgcccccaccgctgggcggcgctgcggggcacccaggctctgcagtcagcgccgcgccgggaatcctgtacccgggcgggaataagtaccagaccattgacaactaccagccgtacccgtgcgcagaggacgaggagtgcggcactgatgagtactgcgctagtcccacccgcggaggggacgcaggcgtgcaaatctgtctcgcctgcaggaagcgccgaaaacgctgcatgcgtcacgctatgtgctgccccgggaattactgcaaaaatggaatatgtgtgtcttctgatcaaaatcatttccgaggagaaattgaggaaaccatcactgaaagctttggtaatgatcatagcaccttggatgggtattccagaagaaccaccttgtcttcaaaaatgtatcacaccaaaggacaagaaggttctgtttgtctccggtcatcagactgtgcctcaggattgtgttgtgctagacacttctggtccaagatctgtaaacctgtcctgaaagaaggtcaagtgtgtaccaagcataggagaaaaggctctcatggactagaaatattccagcgttgttactgtggagaaggtctgtcttgccggatacagaaagatcaccatcaagccagtaattcttctaggcttcacacttgtcagagacactaaaccagctatccaaatgcagtgaactccttttatataatagatgctatgaaaaccttttatgaccttcatcaactcaatcctaaggatatacaagttctgtggtttcagttaagcattccaataacaccttccaaaaacctggagtgtaagagctttgtttctttatggaactcccctgtgattgcagtaaattactgtattgtaaattctcagtgtggcacttacctgtaaatgcaatgaaacttttaattatttttctaaaggtgctgcactgcctatttttcctcttgttatgtaaatttttgtacacattgattgttatcttgactgacaaatattctatattgaactgaagtaaatcatttcagcttatagttcttaaaagcataaccctttaccccatttaattctagagtctagaacgcaaggatctcttggaatgacaaatgataggtacctaaaatgtaacatgaaaatactagcttattttctgaaatgtactatcttaatgcttaaattatatttccctttaggctgtgatagtttttgaaataaaatttaacatttaatatcatgaaatgttataagtagacat

SEQ ID NO: 35 - DNA, 1122 bp, Homo sapiens
gcgggtctcgcttgggttccgctaatttctgtcctgaggcgtgagactgagttcatagggtcctgggtccccgaaccaggaagggttgagggaacacaatctgcaagcccccgcgacccaagtgaggggccccgtgttggggtcctccctccctttgcattcccacccctccgggctttgcgtcttcctggggaccccctcgccgggagatggccgcgttgatgcggagcaaggattcgtcctgctgcctgctcctactggccgcggtgctgatggtggagagctcacagatcggcagttcgcgggccaaactcaactccatcaagtcctctctgggcggggagacgcctggtcaggccgccaatcgatctgcgggcatgtaccaaggactggcattcggcggcagtaagaagggcaaaaacctggggcaggcctacccttgtagcagtgataaggagtgtgaagttgggaggtattgccacagtccccaccaaggatcatcggcctgcatggtgtgtcggagaaaaaagaagcgctgccaccgagatggcatgtgctgccccagtacccgctgcaataatggcatctgtatcccagttactgaaagcatcttaacccctcacatcccggctctggatggtactcggcacagagatcgaaaccacggtcattactcaaaccatgacttgggatggcagaatctaggaagaccacacactaagatgtcacatataaaagggcatgaaggagacccctgcctacgatcatcagactgcattgaagggttttgctgtgctcgtcatttctggaccaaaatctgcaaaccagtgctccatcagggggaagtctgtaccaaacaacgcaagaagggttctcatgggctggaaattttccagcgttgcgactgtgcgaagggcctgtcttgcaaagtatggaaagatgccacctactcctccaaagccagactccatgtgtgtcagaaaatttgatcaccattgaggaacatcatcaattgcagactgtgaagttgtgtatttaatgcattatagcatggtggaaaataaggttcagatgcagaagaatggctaaaataagaaacgtgataagaatatagatgatcac

SEQ ID NO: 36 - DNA, 2124 bp, Homo sapiens
  misc_feature (1432)..(1432): unknown nucleotide
ctatcacaatgagaccaacacagacacgaaggttggaaataataccatccatgtgcaccgagaaattcacaagataaccaacaaccagactggacaaatggtcttttcagagacagttatcacatctgtgggagacgaagaaggcagaaggagccacgagtgcatcatcgacgaggactgtgggcccagcatgtactgccagtttgccagcttccagtacacctgccagccatgccggggccagaggatgctctgcacccgggacagtgagtgctgtggagaccagctgtgtgtctggggtcactgcaccaaaatggccaccaggggcagcaatgggaccatctgtgacaaccagagggactgccagccggggctgtgctgtgccttccagagaggcctgctgttccctgtgtgcacacccctgcccgtggagggcgagctttgccatgaccccgccagccggcttctggacctcatcacctgggagctagagcctgatggagccttggaccgatgcccttgtgccagtggcctcctctgccagccccacagccacagcctggtgtatgtgtgcaagccgaccttcgtggggagccgtgaccaagatggggagatcctgctgcccagagaggtccccgatgagtatgaagttggcagcttcatggaggaggtgcgccaggagctggaggacctggagaggagcctgactgaagagatggcgctgggggagcctgcggctgccgccgctgcactgctgggaggggaagagatttagatctggaccaggctgtgggtagatgtgcaatagaaatagctaatttatttccccaggtgtgtgctttaggcgtgggctgaccaggcttcttcctacatcttcttcccagtaagtttcccctctggcttgacagcatgaggtgttgtgcatttgttcagctcccccaggctgttctccaggcttcacagtctggtgcttgggagagtcaggcagggttaaactgcaggagcagtttgccacccctgtccagattattggctgctttgcctctaccagttggcagacagccgtttgttctacatggctttgataattgtttgaggggaggagatggaaacaatgtggagtctccctctgattggttttggggaaatgtggagaagagtgccctgctttgcaaacatcaacctggcaaaaatgcaacaaatgaattttccacgcagttctttccatgggcataggtaagctgtgccttcagctgttgcagatgaaatgttctgttcaccctgcattacatgtgtttattcatccagcagtgttgctcagctcctacctctgtgccagggcagcattttcatatccaagatcaattccctctctcagcacagcctggggagggggtcattgttctcctcgtccatcagggatctcagaggnctcagagactgcaagctgcttgcccaagtcacacagctagtgaagaccagagcagtttcatctggttgtgactctaagctcagtgctctctccactaccccacaccagccttggtgccaccaaaagtgctccccaaaaggaaggagaatgggatttttcttttgaggcatgcacatctggaattaaggtcaaactaattctcacatccctctaaaagtaaactactgttaggaacagcagtgttctcacagtgtggggcagccgtccttctaatgaagacaatgatattgacactgtccctctttggcagttgcattagtaactttgaaaggtatatgactgagcgtagcatacaggttaacctgcagaaacagtacttaggtaattgtagggcgaggattataaatgaaatttgcaaaatcacttagcagcaactgaagacaattatcaaccacgtggagaaaatcaaaccgagcagggctgtgtgaaacatggttgtaatatgcgactgcgaacactgaactctacgccactccacaaatgatgttttcaggtgtcatggactgttgccaccatgtattcatccagagttcttaaagtttaaagttgcacatgattgtataagcatgctttctttgagttttaaattatgtataaacataagttgcatttagaaatcaagcataaatcac

SEQ ID NO: 37 - DNA, 816 bp, Homo sapiens
agacgacgtgctgagctgccagcttagtggaagctctgctctgggtggagagcagcctcgctttggtgacgcacagtgctgggaccctccaggagccccgggattgaaggatggtggcggccgtcctgctggggctgagctggctctgctctcccctgggagctctggtcctggacttcaacaacatcaggagctctgctgacctgcatggggcccggaagggctcacagtgcctgtctgacacggactgcaataccagaaagttctgcctccagccccgcgatgagaagccgttctgtgctacatgtcgtgggttgcggaggaggtgccagcgagatgccatgtgctgccctgggacactctgtgtgaacgatgtttgtactacgatggaagatgcaaccccaatattagaaaggcagcttgatgagcaagatggcacacatgcagaaggaacaactgggcacccagtccaggaaaaccaacccaaaaggaagccaagtattaagaaatcacaaggcaggaagggacaagagggagaaagttgtctgagaacttttgactgtggccctggactttgctgtgctcgtcatttttggacgaaaatttgtaagccagtccttttggagggacaggtctgctccagaagagggcataaagacactgctcaagctccagaaatcttccagcgttgcgactgtggccctggactactgtgtcgaagccaattgaccagcaatcggcagcatgctcgattaagagtatgccaaaaaatagaaaagctataaatatttcaaaataaagaagaatccacattgc

SEQ ID NO: 38 - DNA, 1476 bp, Homo sapiens
acggggcctgggcggsaggggcggtggctggagctcggtaaagctcgtgggaccccattgggggaatttgatccaaggaagcggtgattgccgggggaggagaagctcccagatccttgtgtccacttgcagcgggggaggcggagacgcggagcgggccttttggcgtccactgcgcggctgcaccctgccccatcctgccgggatcatggtctgcggcagcccgggagggatgctgctgctgcgggccgggctgcttgccctggctgctctctgcctgctccgggtgcccggggctcgggctgcagcctgtgagcccgtccgcatccccctgtgcaagtccctgccctggaacatgactaagatgcccaaccacctgcaccacagcactcaggccaacgccatcctggccatcgagcagttcgaaggtctgctgggcacccactgcagccccgatctgctcttcttcctctgtgccatgtacgcgcccatctgcaccattgacttccagcacgagcccatcaacccctgtaagtctgtgtgcgagcgggcccggcagggctgtgagcccatactcatcaagtaccgccactcgtggccggagaacctggcctgcgaggagctgccagtgtacgacaggggcgtgtgcatctctcccgaggccatcgttactgcggacggagctgattttcctatggattctagtaacggaaactgtagaggggcaagcagtgaacgctgtaaatgtaagcctattagagctacacagaagacctatttccggaacaattacaactatgtcattcgggctaaagttaaagagataaagactaagtgccatgatgtgactgcagtagtggaggtgaaggagattctaaagtcctctctggtaaacattccacgggacactgtcaacctctataccagctctggctgcctctgccctccacttaatgttaatgaggaatatatcatcatgggctatgaagatgaggaacgttccagattactcttggtggaaggctctatagctgagaagtggaaggatcgactcggtaaaaaagttaagcgctgggatatgaagcttcgtcatcttggactcagtaaaagtgattctagcaatagtgattccactcagagtcagaagtctggcaggaactcgaacccccggcaagcacgcaactaaatcccgaaatacaaaaagtaacacagtggacttcctattaagacttacttgcattgctggactagcaaaggaaaattgcactattgcacatcatattctattgtttactataaaaatcatgtgataactgattattacttctgtttctcttttggtttctgcttctctcttctctcaacccctttgtaatggtttgggggcagactcttaagtatattgtgagttttctatttcactaatcatgagaaaaactgttcttttgcaataataataaattaaacatgctgtta

SEQ ID NO: 39 - DNA, 882 bp, Homo sapiens
  misc_feature (615)..(615): unknown nucleotide
gaattcgttcagcctggttaagtccaagctggctcattctgctcccccgggtcggagccccccggagctgcgcgcgggcttgcagcgcctcgcccgcgctgtcctcccggtgtcccgcttctccgcgccccagccgccggctgccagcttttcggggccccgagtcgcacccagcgaagagagcgggcccgggacaagctcgaactccggccgcctcgcccttaaccagctccgtccctctaccccctaggggtcgcgcccacgatgctgcagggccctggctcgctgctgctgctcttcctcgcctcgcactgctgcctgggctcggcgcgcgggctcttcctctttggccagcccgacttctcctacaagcgcagcaattgcaagcccatcccggccaacctgcagctgtgccacggcatcgaataccagaacatgcggctgcccaacctgctgggccacgagaccatgaaggaggtgctggagcaggccggcgcttggatcccgctggtcatgaagcagtgccacccggacaccaagaagttcctgtgctcgctcttcgcccccgtctgcctcgatgacctagacgagaccatccagccatgccactctcgntgcgtgcaggtgaaggatcgctgcgccccggtcatgtccgccttcccctggcccgacatgcttgagtgcgaccgtttcccccaggacaacgacctttgcatccccctcgctagcagcgaccacctcctgccagccaccgaggaagctccaaaggtatgtgaagcctgcaaaaataaaaatgatgatgacaacgacataatggaaacgctttgtaaaaatgattttgcactgaaaataaaagtgaaggagataacctacatcaaccgt

SEQ ID NO: 40 - DNA, 10363 bp, Homo sapiens
aggtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtatgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcgagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggggcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcttcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggtcttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccggtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaaaagagaggaagaatgaaactaagaaaattctatgttaattacaactgctatatagacattttgtttcaaatgaaactttaaaagactgaaaaattttgtaaataggtttgattcttgttagagggtttttgttctggaagccatatttgatagtatactttgtcttcactggtcttattttgggaggcactcttgatggttaggaaaaaaatagtaaagccaagtatgtttgtacagtatgttttacatgtatttaaagtagcatcccatcccaacttcctttaattattgcttgtcttaaaataatgaacactacagatagaaaatatgatatattgctgttatcaatcatttctagattataaactgactaaacttacatcagggaaaaattggtatttatgcaaaaaaaaatgtttttgtccttgtgagtccatctaacatcataattaatcatgtggctgtgaaattcacagtaatatggttcccgatgaacaagtttacccagcctgctttgctttactgcatgaatgaaactgatggttcaatttcagaagtaatgattaacagttatgtggtcacatgatgtgcatagagatagctacagtgtaataatttacactattttgtgctccaaacaaaacaaaaatctgtgtaactgtaaaacattgaatgaaactattttacctgaactagattttatctgaaagtaggtagaatttttgctatgctgtaatttgttgtatattctggtatttgaggtgagatggctgctcttttattaatgagacatgaattgtgtctcaacagaaactaaatgaacatttcagaataaattattgctgtatgtaaactgttactgaaattggtatttgtttgaagggtcttgtttcacatttgtattaataattgtttaaaatgcctcttttaaaagcttatataaatttttttcttcagcttctatgcattaagagtaaaattcctcttactgtaataaaaacaattgaagaagactgttgccacttaaccattccatgcgttggcacttatctattcctgaaatttcttttatgtgattagctcatcttgatttttaatatttttccacttaaacttttttttcttactccactggagctcagtaaaagtaaattcatgtaatagcaatgcaagcagcctagcacagactaagcattgagcataataggcccacataatttcctctttcttaatattatagaattctgtacttgaaattgattcttagacattgcagtctcttcgaggctttacagtgtaaactgtcttgccccttcatcttcttgttgcaactgggtctgacatgaacactttttatcaccctgtatgttagggcaagatctcagcagtgaagtataatcagcactttgccatgctcagaaaattcaaatcacatggaactttagaggtagatttaatacgattaagatattcagaagtatattttagaatccctgcctgttaaggaaactttatttgtggtaggtacagttctggggtacatgttaagtgtccccttatacagtggagggaagtcttccttcctgaaggaaaataaactgacacttattaactaagataatttacttaatatatcttccctgatttgttttaaaagatcagagggtgactgatgatacatgcatacatatttgttgaataaatgaaaatttatttttagtgataagattcatacactctgtatttggggagggaaaacctttttaagcatggtggggcactcagataggagtgaatacacctacctggtg

SEQ ID NO: 41 - DNA, 3381 bp, Homo sapiens
gggcccggggtcccgccaccaccgcgcgcgggacagattgattcactttggagctgtaagtactgatgtattagggtgcagcgctcattgttccttgacgcagagtcccaaaatgaatatccaagagcagggtttccccttggacctcggagcaagtttcaccgaagatgctccccgacccccagtgcctggtgaggagggagaactggtgtccacagacccgaggcccgccagctacagtttctgctccgggaaaggtgttggcattaaaggtgagacttcgacggccactccgaggcgctcggatctggacctggggtatgagcctgagggcagtgcctcccccaccccaccatacttgaagtgggctgagtcactgcattccctgctggatgaccaagatgggataagcctgttcaggactttcctgaagcaggagggctgtgccgacttgctggacttctggtttgcctgcactggcttcaggaagctggagccctgtgactcgaacgaggagaagaggctgaagctggcgagagccatctaccgaaagtacattcttgataacaatggcatcgtgtcccggcagaccaagccagccaccaagagcttcataaagggctgcatcatgaagcagctgatcgatcctgccatgtttgaccaggcccagaccgaaatccaggccactatggaggaaaacacctatccctccttccttaagtctgatatttatttggaatatacgaggacaggctcggagagccccaaagtctgtagtgaccagagctctgggtcagggacagggaagggcatatctggatacctgccgaccttaaatgaagatgaggaatggaagtgtgaccaggacatggatgaggacgatggcagagacgctgctccccccggaagactccctcagaagctgctcctggagacagctgccccgagggtctcctccagtagacggtacagcgaaggcagagagttcaggtatggatcctggcgggagccagtcaacccctattatgtcaatgccggctatgccctggccccagccaccagtgccaacgacagcgagcagcagagcctgtccagcgatgcagacaccctgtccctcacggacagcagcgtggatgggatccccccatacaggatccgtaagcagcaccgcagggagatgcaggagagcgtgcaggtcaatgggcgggtgcccctacctcacattcccgtaagtaccgggtgccgaaggaggtccgcgtggagcctcagaagttcgcggaggagctcatccaccgcctggaggctgtgcagcgcacgcgggaggccgaggagaagctggaggagcggctgaagcgcgtgcgcatggaggaggaaggtgaggacggcgatccatcgtcagggcccccagggccgtgtcacaagctgcctcccgcccccgcttggcaccacttcccgccccgctgtgtggacatgggctgtgccgggctccgggatgcacacgaggagaaccctgagagcatcctggacgagcacgtacagcgtgtgctgaggacacctggccgccagtcgcctgggcctggccatcgctccccggacagtgggcacgtggccaagatgccagtggcactggggggtgccgcctcggggcacgggaagcacgtacccaagtcaggggcgaagctggacgcggccggcctgcaccaccaccgacacgtccaccaccacgtccaccacagcacagcccggcccaaggagcaggtggaggccgaggccacccgcagggcccagagcagcttcgcctggggcctggaaccacacagccatggggcaaggtcccgaggctactcagagagtgttggcgctgcccccaacgccagtgatggcctcgcccacagtgggaaggtgggcgtggcgtgcaaaagaaatgccaagaaggctgagtcggggaagagcgccagcaccgaggtgccaggtgcctcggaggatgcggagaagaaccagaaaatcatgcagtggatcattgagggggaaaaggagatcagcaggcaccgcaggaccggccacgggtcttcggggacgaggaagccacagccccatgagaactccagacccttgtcccttgagcacccctgggccggccctcagctccggacctccgtgcagccctcccacctcttcatccaagaccccaccatgccaccccacccagctcccaaccccctaacccagctggaggaggcgcgccgacgtctggaggaggaagaaaagagagccagccgagcaccctccaagcagaggtatgtgcaggaggttatgcggcggggacgcgcctgcgtcaggccagcgtgcgcgccggtgctgcacgtggtaccagccgtgtcggacatggagctctccgagacagagacaagatcgcagaggaaggtgggcggcgggagtgcccagccgtgtgacagcatcgttgtggcgtactacttctgcggggaacccatcccctaccgcaccctggtgaggggccgcgctgtcaccctgggccagttcaaggagctgctgaccaaaaagggcagctacagatactacttcaagaaagtgagcgacgagtttgactgtggggtggtgtttgaggaggttcgagaggacgaggccgtcctgcccgtctttgaggagaagatcatcggcaaagtggagaaggtggactgataggctggtgggctggccgctgtgccaggcgaggcccttggcgggcacgggtgtcacggccaggcagatgacctcgtactcaggagcccgatggggaacagtgttgggtgtaccacccatccctgtggtctacccgtgtctagaggcaggtagggggtccctccaagtggtccacaagcttctgtcctgcccccaaggaggcagcctggaccactcctcatagcaatacttggagggcccagcccaagtgaggcagccgaggtccctgctgccagcttcaggtgacccccccccatcccccggcacctcccttgggcacgtgtgctgggatctactttccctctgggatttgcccacgtacccaggtctgggtggggcccaggcccggatgcagaggcctgcagggcctctgtcaattgtacgcgccaccgagtgccttcaacacagcttgtctcttgcctgccactgtgtgaatcggcgacggagcactgcacctgcctccagccgccggctgtgcagtcctgggtcctcctttctgagggcccgtgtaaatatgtacatttctcaggctaggccagcaggggctgcccgagtctgtttttcatgcgatgacacttgtacaattaattatcttttcaaaggtacttggataataatgaaataaaactgtttttgaacctg

SEQ ID NO: 42 - DNA, 2538 bp, Homo sapiens
gttcagatgagcccctgctgacttgagagagacagagagaccacgccgattgctgagaggaactggaagaagaagagattccagactcagtgggaagagctccctcaccatgagtagcgctatgttggtgacttgcctcccggaccccagcagcagcttccgtgaggatgccccgcggcccccagtgccaggggaagaaggggagaccccaccgtgtcaccatggtgggcaagggccaggtcaccaaacccatgtctgtctcttccaacaccaggcggaacgaagatgggttggggagccggaggggcgggcatctccggattcccctctgacccggtggaccaagtccttacactccttattgggcgatcaagacggtgcttacctgttccgaactttcctggagagggagaaatgcgtggataccttagacttctggtttgcctgcaatggattcaggcagatgaacctgaaggataccaaaactttacgagtagccaaagcgatctacaaaaggtacattgagaacaacagcattgtctccaagcagctgaagcctgccaccaagacctacataagagatggcatcaagaagcagcagattgattccatcatgtttgaccaggcgcagaccgagatccagtcggtgatggaggaaaatgcctaccagatgtttttgacttctgatatatacctcgaatatgtgaggagtgggggagaaaacacagcttacatgagtaatgggggactcgggagcctaaaggtcgtgtgtggctatctccccaccttgaatgaagaagaggagtggacttgtgccgacttcaagtgcaaactttcgccaaccgtggttggcttgtccagcaaaactctgagggccacggcgagtgtgaggtccacggaaactgttgacagtggatacaggtccttcaagaggagcgatcctgttaatccttatcacataggttctggctatgtctttgcaccagccaccagcgccaacgacagtgagatatccagtgatgcgctgacggatgattccatgtccatgacggacagcagtgtagatggaattcctccttatcgtgtgggcagtaagaaacagctccagagagaaatgcatcgcagtgtgaaggccaatggcagagtgtctctacctcatttcccgagaacccaccgcctgcccaaggagatgacccccgtggaacccgccacctttgcagctgagctgatctcgaggctggaaaagctgaagctggagttggagagccgccacagcctggaggagcgcctgcagcagatccgagaggatgaagagagagagggctccgagctcacactcaattcgcgggagggggcgcccacgcagcaccccctctccctactgccctccggcagctacgaggaagacccgcagacgatactggacgatcacctgtccagggtcctcaagacccctggctgccagtctccgggcgtaggccgctatagccctcgctcccgctccccggaccaccaccaccaccaccattcgcagtaccactccctgctcccgcccggtggcaagctgcctcccgcggccgcctcgccgggcgcctgccccctcctcgggggcaaaggctttgtgaccaagcagacgacgaagcatgtccaccaccactacatccaccaccatgccgtccccaagaccaaggaggagatcgaggcggaggccacgcagcgggtgcactgcttctgccctgggggcagcgagtattactgctactcgaaatgcaaaagccactccaaggctccggaaaccatgcccagcgagcagtttggtgcccaaagcacaaaaaaggcctaccccttggagtctgcccgctcgtctccaggcgaacgagccagccggcaccatctgtgggggggcaacagcgggcacccccgcaccaccccccgtgcccacctgttcacccaggaccctgcgatgccttccctgaccccacccaacacgctggctcatctggaggaggcctgtcgcaggctagctgaggtgtcgaagcccccaaagcagcggtgctgtgtggccagtcagcagagggacaggaatcattcggccactgttcagacgggagccacacccttctccaatccaagcctggctccagaagatcacaaagagccaaagaaactggcaggtgtccacgcgctccaggccagtgagttggttgtcacttactttttctgtggggaagaaattccataccggaggatgctgaaggctcagagcttgaccctgggccactttaaagagcagctcagcaaaaagggaaattataggtattacttcaaaaaagcaagcgatgagtttgcctgtggagcggtgtttgaggagatctgggaggatgagacggtgctcccgatgtatgaaggccggattctgggcaaagtggagcggatcgattgagccctggggtctggctttggtgaactgttggagcccgaagctcttgtgaactgtcttggctgtgagcaactgcgacaaaacattttgaaggaaaa

SEQ ID NO: 43 - DNA, 2557 bp, Homo sapiens
attccggctcccgcggctgcaggcgcgcggctagagtgcctggcgggctccggcttccgcgtccgccccggccccggtccagacttagtcttcagctccgcgcccgctccgccgcggcccaccgcgcccgccggcagccgagcccccagcgacgcccgcacagctccgggtgcccagacagggggccatgccgtgccggagggaggaggaagaggaagccggcgaggaggcggagggggaggaagaggaggaggacagcttcctcctactgcagcagtcagtggcgctgggcagctcgggcgaggtggaccggctggtggcccagatcggcgagacgctgcagctggacgcggcgcagcacagcccggcctcgccgtgcgggcccccgggggcgccgctgcgggccccggggcccctggctgcggcggtgccggcggacaaggccaggtccccggcggtgccgctgctgctgccgcccgcgttggcggagactgtgggcccggcgccccctggggtcctgcgctgcgccctgggggaccgcggccgcgtgcggggccgcgctgcgccctactgcgtggccgagctcgccacaggccccagcgcgctgtccccactgccccctcaggccgaccttgatgggcctccgggagctggcaagcagggcatcccgcagccgctgtcgggtccgtgccggcgaggatggctccggggcgccgccgcctcccgccgcctgcagcagcgacgcgggtcccaaccagaaacccgcacaggcgacgacgacccgcaccggcttctgcagcagctagtgctctctggaaacctcatcaaggaggccgtgcgaaggcttcattcgcgacggctgcagttacgtgcaaagcttccccaacgcccgctcctgggacctctgtcggccccggtgcatgaacccccttcgcctcgcagccctcgcgcggcctgcagtgaccctggcgcctccgggagggcgcagctcagaactggcgacggcgttcttgtgcctggcagctaacacgcccggggtggccacagcgccagcctcagactggagggcaaggggttcccttgagggctgcagttctactcaggctggtggagaactctggcttttggaagcgagagtaaaaagctaatgacgaggaaccgaaaaatcgcgagtgtttcgcgggtaactggggttgagggccaaaatatttggaatgaaggactttggccctatttaaggcagattttacagagcgcacctcaaacgtacaagtcagtaggactccttatttggcgtgacccgacctggccgcggagcctgcatttcctcgcagcctctcagtgccctccagccccgcgaccatgtggccacaatccacgcttctccggatcgcggtgcgccggaaccacggaggatgatgccagttacttgctttaccttttcagggctggctcctgatccactttgggggaggagaacatgagtagataatttcagggtgcagcccaatctgccagacttaaaaaaaccatcttgtgtctttggaggtgctgcttaataccaaacatgcggtgccatgaagggaccctttgggggttgaataggagttaacccctgcgctctctttgcaactgtctctcttctcagagtggtgggggaaggctgtacgacacgggtggggaaaggaggtgggggcggggagtattgaatggtggtggaagggtagagaggcgcggagtgaaccccacgccctgtctaaagtgtattttcagagccggcccgcctctcctcggttcaaggtcactgtttcctgggcacgcactgggttgcgggacagagtagccaggttctgccggtgctcggagaagagcgcagtgttttgcaagtgctggagtctcctgaggacacgcgcgtcgccgccaccgcgggtgtgggaaagcgcggacgtgctgggcggctgtgcttcggtaggcgaccaccgcccctggccgcgctccgggctttcacggaaactcccgagaccgggccctgggttcctcctctcctactcggctctgcagtcctactcaagcgggtggctctgggatcctgggggcctgggttgggggctagggagacgccatgtgatggacactccagggacacacagcctagcacagcagcttataatgggctctccggggccatttgcaataacagctgcaattccctggatagacgagttgatttcctccctctgcccctcccccagccatgccagctggcctttgtaagtgcaggaaaccgagtagaaaatgtgaccctccaaatggagaagctgcaggctttgccattgtgaaccatggtgaagtgcttggaacatactgttcactcactctaaaggcgctgagactgtgctgttgttctcgtttttatagtcaatggcttgttcatcatccagatgtggctactgacatatctacacttcgcaccggagtgtctggaattgtggctatcctgattataggattttaacttaactgaaatgcctgctttgaataaatgtgttgggttttt

SEQ ID NO: 44 - DNA, 1231 bp, Homo sapiens
gaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcag

SEQ ID NO: 45 - DNA, 2980 bp, Homo sapiens
  misc_feature (2974)..(2974): unknown nucleotide
ggccgctcctgctgctgctactgccgccgccgcagcggctgctcgggctgagcacgccccggaacaggccgccgcgcgctgcgcgccggacccgctgcccctgccggcccggccgggtcgggcggcccagggaccgacagacttgacaacggtgacagcactggggcggcaccttcctacttctgcccagccacagccctcccctcacagttgagcacctgtttgcctgaagttaatttccagaagcaggagtccccagagccaggcagggggatgaaccgcgagggagctcccgggaagagtccggaggagatgtacattcagcagaaggtccgagtgctgctcatgctgcggaagatgggatcaaacctgacagccagcgaggaggagttcctgcgcacctatgcaggggtggtcaacagccagctcagccagctgcctccgcactccatcgaccagggtgcagaggacgtggtgatggcgttttccaggtcggagacggaagaccggaggcagtagctgcaaagcccttggaacaccctggatgctgttgaagggccaagagatctgtgtggctcctgggccggctgaatggcagcagccccccttgccccacctcccccttccctacccaaccctgccctgccccaccccacctcacagctactcagtggggctggcatcaagggagacaccagtggtgcgtttataattggcttaaagggatggacttgtgattggctgcaggaagaaacttttttattttttaaatcttgaccaacagaaaccttttatttttatttctgactcttattttttaaaaaatttgcgcctcggtatctggcttccctggaagctctccgagctctggtgctttagttaggtcatttttttagaaatgtgaagaggtctgattggctgcttaaactggaaagggactgtgattggctggttaatgggaaacggtttttttctttggctgcaggtgttctgctgatatcaacagcttccctattttgaatgcagaaaacagggtctgggacattagtcgttatatttgacttgaaaagaaagaaaccaagtgcgctttgcaatatttattacacaaagaacttgctgctgccttcacatttggggtttgtgtttgattggctttcgatgcgtgtgtttggtttcccattggttcacctgtgactcctgttgccatggattcacccccctctgctgccggctctgggcctgagggtccacctggagagtacatttgctttaatgagtgcacctgcctccaccagcaaggggaccccgagaaccctgagcagggtccacagctggaaagttgggcccctgaggagctttgtgtcgtcttgaacgagcagcccagggcctagaggtaaccgttaggcgggatttatgtgcactgcctgcatgagctggcaaccagccagcgtcccttggtgagaaagggattgctgaggcaccgtccaggccccaccggccaggccgcgcccagcagaggcgtactacccagctctgtcctcttggccatccttctgtgtaccacttcctgaggcctcattttgggggtcatcttggaaaggggaggagcttctcccagtgtgagaccccaaagactctggaggtcatctggcggaggtctctgggagcccagaacccacataaaagccccagcttggctcacaaggcccaggagacctccagctaaacaccaacccctgacctaccccagccaggctcctacctgtctgctgccagcacagtaggtcccggccagctctggagttctctcatcggaggcccatgccctccactccactgcctttggaagggtctctctccaggtcagcctggaagggacagtatcgtttgtttatgaaatgccactgggacagctggctgggccttcaccaagcaagtcccttcagactggcccttaagccaaactcaggcccagaattgcagttcagaatggcagtcctggaggcagggggtgaggggcaggtctagtgttcctgcaccaaacctaagtccttccacctgccacccccttccctgggagggaggtggtcctcctatctccctggctcactggcaggtgtgggatctggggagagcggctggagaaagatgcagtcctcaggaagggggccgccaccctcccctatgctggtagatgctgaggcccctaggtgcccagggccagtgggaccctctcagaaccaaatctttcccctttctcggggcttggggctcgggccgtaggggctcctgagtgtcatgaagtgcacaggagccaaatgaccgagccctggagagccccatggtgggtaggtggttcgtgctgtgctctggcaccatcagcctgttccagaaggaggattcgagcatcaggctaagaccctgtgtcctccaccatgcactcacccctagccctggttagctgacagtcagctgtggggaacacagctacaaccctaccctggcagggacctgagagcatctcaggaggggcagcgcatgtgtgcatgtgctgtgtgagtgagcacacccgtgtgcacactcatacacatgtgcacacacacgcactctccccgctcaggggcctggaggtctggctgagcccctggggaaaggtgagttctttcatctccctcctccaggtcggagtgcctggagtcaggtgtcgaggccacattgctggctgccccctctttgtagctcctataaagggcccacacctggtggatacctggttgagcgtgtggtctctgccccagcctgtccttgtcacgatcacaggccttgcttttgtaacaatgatgaccccggcctgtctcatcttctgaagaggaaaagtcaaagtgttgctgtggctccatatttcaactaaaaatatatctgttggagaaagaaattaacaataaagaattttcataggttaaaaaaaaaaaanaaaaag

SEQ ID NO: 46 - DNA, 761 bp, Homo sapiens
ggcggcaggaccagcatgcaccaccgaaacgactcccagaggctggggaaagctggctgcccgccagagccgtcgttgcaaatggcaaatactaatttcctctccaccttatcccctgaacactgcagacctttggcgggggaatgcatgaacaagctcaaatgcggcgctgctgaagcagagataatgaatctccccgagcgcgtggggactttttccgctatcccggctttagggggcatctcattacctccaggggtcatcgtcatgacagcccttcactcccccgcagcagcctcagcagccgtcacagacagtgcgtttcaaattgccaatctggcagactgcccgcagaatcattcctcctcctcctcgtcctcctcagggggagctggcggagccaacccagccaagaagaagaggaaaaggtgtggggtctgcgtgccctgcaagaggctcatcaactgtggcgtctgcagcagttgcaggaaccgcaaaacgggacaccagatctgcaaatttagaaaatgtgaagagctaaagaaaaaacctggcacttcactagagagaacacctgttcccagcgctgaagcattccgatggttcttttaaagcagtagtatatcttattttcaaggcatttggaaatgaagggcaaactaatgtcttgttttaagaaactgcttagtccaccactgaagaaaatatccagaaattattttcattttatgtatagggatttcttcaaaaaaaaaaaaaaa

SEQ ID NO: 47 - DNA, 2905 bp, Homo sapiens
cctctgaaggttctagaatcgatagtgaattcgtgggacgggaagaggaagcactgcctttacttcagtgggaatctcggcctcagcctgcaagccaagtgttcacagtgaaaaaagcaagagaataagctaatactcctgtcctgaacaaggcagcggctccttggtaaagctactccttgatcgatcctttgcaccggattgttcaaagtggaccccaggggagaagtcggagcaaagaacttaccaccaagcagtccaagaggcccagaagcaaacctggaggtgagacccaaagaaagctggaaccatgctgactttgtacactgtgaggacacagagtctgttcctggaaagcccagtgtcaacgcagatgaggaagtcggaggtccccaaatctgccgtgtatgtggggacaaggccactggctatcacttcaatgtcatgacatgtgaaggatgcaagggctttttcaggagggccatgaaacgcaacgcccggctgaggtgccccttccggaagggcgcctgcgagatcacccggaagacccggcgacagtgccaggcctgccgcctgcgcaagtgcctggagagcggcatgaagaaggagatgatcatgtccgacgaggccgtggaggagaggcgggccttgatcaagcggaagaaaagtgaacggacagggactcagccactgggagtgcaggggctgacagaggagcagcggatgatgatcagggagctgatggacgctcagatgaaaacctttgacactaccttctcccatttcaagaatttccggctgccaggggtgcttagcagtggctgcgagttgccagagtctctgcaggccccatcgagggaagaagctgccaagtggagccaggtccggaaagatctgtgctctttgaaggtctctctgcagctgcggggggaggatggcagtgtctggaactacaaacccccagccgacagtggcgggaaagagatcttctccctgctgccccacatggctgacatgtcaacctacatgttcaaaggcatcatcagctttgccaaagtcatctcctacttcagggacttgcccatcgaggaccagatctccctgctgaagggggccgctttcgagctgtgtcaactgagattcaacacagtgttcaacgcggagactggaacctgggagtgtggccggctgtcctactgcttggaagacactgcaggtggcttccagcaacttctactggagcccatgctgaaattccactacatgctgaagaagctgcagctgcatgaggaggagtatgtgctgatgcaggccatctccctcttctccccagaccgcccaggtgtgctgcagcaccgcgtggtggaccagctgcaggagcaattcgccattactctgaagtcctacattgaatgcaatcggccccagcctgctcataggttcttgttcctgaagatcatggctatgctcaccgagctccgcagcatcaatgctcagcacacccagcggctgctgcgcatccaggacatacacccctttgctacgcccctcatgcaggagttgttcggcatcacaggtagctgagcggctgcccttgggtgacacctccgagaggcagccagacccagagccctctgagccgccactcccgggccaagacagatggacactgccaagagccgacaatgccctgctggcctgtctccctagggaattcctgctatgacagctggctagcattcctcaggaaggacatgggtgccccccacccccagttcagtctgtagggagtgaagccacagactcttacgtggagagtgcactgacctgtaggtcaggaccatcagagaggcaaggttgccctttccttttaaaaggccctgtggtctggggagaaatccctcagatcccactaaagtgtcaaggtgtggaagggaccaagcgaccaaggataggccatctggggtctatgcccacatacccacgtttgttcgcttcctgagtcttttcattgctacctctaatagtcctgtctcccacttcccactcgttcccctcctcttccgagctgctttgtgggctcaaggcctgtactcatcggcaggtgcatgagtatctgtgggagtcctctagagagatgagaagccaggaggcctgcaccaaatgtcagaagcttggcatgacctcattccggccacatcattctgtgtctctgcatccatttgaacacattattaagcactgataataggtagcctgctgtggggtatacagcattgactcagatatagatcctgagctcacagagtttatagttaaaaaaacaaacagaaacacaaacaatttggatcaaaaggagaaaatgataagtgacaaaagcagcacaaggaatttccctgtgtggatgctgagctgtgatggcaggcactgggtacccaagtgaaggttcccgaggacatgagtctgtaggagcaagggcacaaactgcagctgtgagtgcgtgtgtgtgatttggtgtaggtaggtctgtttgccacttgatggggcctgggtttgttcctggggctggaatgctgggtatgctctgtgacaaggctacgctgacaatcagttaaacacaccggagaagaaccatttacatgcaccttatatttctgtgtacacatctattctcaaagctaaagggtatgaaagtgcctgccttgtttatagccacttgtgagtaaaaatttttttgcattttcacaaattatactttatataaggcattccacacctaagaactagttttgggaaatgtagccctgggtttaatgtcaaatcaaggcaaaaggaattaaataatgtacttttggctaaaaaaaaaaaaaaaaaaaaaaaaaaaaaaaaaaaaaaaaaaaaaaaa

SEQ ID NO: 48 - DNA, 3089 bp, Homo sapiens
cccgcaggggttcctccaagatggcggcgcagaggaggagcttgctgcagagtgagcagcagccaagctggacagatgacctgcctctctgccacctctctggggttggctcagcctccaaccgcagctactctgctgatggcaagggcactgagagccacccgccagaggacagctggctcaagttcaggagtgagaacaactgcttcctgtatggggtcttcaacggctatgatggcaaccgagtgaccaacttcgtggcccagcggctgtccgcagagctcctgctgggccagctgaatgccgagcacgccgaggccgatgtgcggcgtgtgctgctgcaggccttcgatgtggtggagaggagcttcctggagtccattgacgacgccttggctgagaaggcaagcctccagtcgcaattgccagagggagtccctcagcaccagctgcctcctcagtatcagaagatccttgagagactcaagacgttagagagggaaatttcgggaggggccatggccgttgtggcggtccttctcaacaacaagctctacgtcgccaatgtcggtacaaaccgtgcacttttatgcaaatcgacagtggatgggttgcaggtgacacagctgaacgtggaccacaccacagagaacgaggatgagctcttccgtctttcgcagctgggcttggatgctggaaagatcaagcaggtggggatcatctgtgggcaggagagcacccggcggatcggggattacaaggttaaatatggctacacggacattgaccttctcagcgctgccaagtccaaaccaatcatcgcagagccagaaatccatggggcacagccgctggatggggtgacgggcttcttggtgctgatgtcggaggggttgtacaaggccctagaggcagcccatgggcctgggcaggccaaccaggagattgctgcgatgattgacactgagtttgccaagcagacctccctggacgcagtggcccaggccgtcgtggaccgggtgaagcgcatccacagcgacaccttcgccagtggtggggagcgtgccaggttctgcccccggcacgaggacatgaccctgctagtgaggaactttggctacccgctgggcgaaatgagccagcccacaccgagcccagccccagctgcaggaggacgagtgtaccctgtgtctgtgccatactccagcgcccagagcaccagcaagaccagcgtgaccctctcccttgtcatgccctcccagggccagatggtcaacggggctcacagtgcttccaccctggacgaagccacccccaccctcaccaaccaaagcccgaccttaaccctgcagtccaccaacacgcacacgcagagcagcagctccagctctgacggaggcctcttccgctcccggcccgcccactcgctcccgcctggcgaggacggtcgtgttgagccctatgtggactttgctgagttttaccgcctctggagcgtggaccatggcgagcagagcgtggtgacagcaccgtagggcagccggagaatgcagcccaagcagggcctggcatggggcaggacagggtccagccttttcctaacatctgcctgtgccacaacggccagcaggtgccccatcctctgcccacagcagactctgtcccatggctctccgggcagtagagtgtgtgagtgcagactggacctgtggttcataccttgtcaccacccgggaagctgaaggccacttcctcccagatggcctcagccaggaccatcgccctttctcagagcagagggccaggtatagaaaccgcagtgggcctgcaagccgcccgagcctccccagcagcctcctacagagcaggaagaggcgccctgtgaaccctgagtgttgcaggcccagcagaccctgctgtcccaagcccacccctcctcccaccatcacctccctcacctcgggacagtagccctccacttctccagcctctcagccctgtgctcctgcatccagagtggaacccaggctggtgtccgcatctgtccctgggccccacccctggacctgccttggttgtgtcatctgttgtaaacgttcaggaggaccagggcagcatctggggcctgggatggccacagaaggggcaggccaggtggaaaggagccagggggaagtggtctaagagacctggaactgccagaggatggcggcctgggcttccccagagccaggcgtgcgggagaggtgaggactggccccggtgggctgaggcaggggccgctgtcgtcaggcctgagccagggtgagctggtgcctgccttgcattttccttctggtgctgtgaagaccataggctggcaggcagctgagatgaactgtctttaccactgatgaggggcctctgccggctgagggtagcaagcaggggttgtgagtcaggctgggggacttgtttgaaagaaagaggagtggaaaatggttccaggagggaagaggttctttgagacacagtaccctgggaggcataggagaagggtcgggccagcccagcccagggcctgagttagactatttcccacatgttctctgccttcagtggggagggggtgccaccagggctgtcggccaggattgccactcctgtttcagaggaagcaggccgagagacttgcaccttggccaagccacacaatcagtggggcagccagagctcagacctgagccattttgtcagtatccaggaccccccggattctccacgccctccccatctcccagtctccctgccccccatgccccagaccggcccaccagggactagccgctgtcgcacagcctctggggtgcttggtctctgcgaagtcaaaggcctgacagctctgtggcctgggaatccattttcctgcggcagagcagggcctggtgtggaaccagggagctgtgggaagccacagcagaaatggaagaaaaacaggtctcagcccagggtcctcgctcactccctcactccccactttgaagccatctctgttctgcaggtgagaggatttaaagtcagtcacaaaggcttgggaacaaaaggaattc

SEQ ID NO: 49 - DNA, 3362 bp, Homo sapiens
aagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttagcaaaccctagccttgcttgttaaaatttttttttttttttttttaagaatatctgtaatggtactgactttgcttgctttgaagtagctctttttttttttttttttttttttttttgcagtaactgttttttaagtctctcgtagtgttaagttatagtgaatactgctacagcaatttctaatttttaagaattgagtaatggtgtagaacactaattaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcatt

SEQ ID NO: 50 - DNA, 1818 bp, Homo sapiens
atggacccggccgaggcggtgctgcaagagaaggcactcaagtttatgtgctctatgcccaggtctctgtggctgggctgctccagcctggcggacagcatgccttcgctgcgatgcctgtataacccagggactggcgcactcacagctttccagaattcctcagagagagaagactgtaataatggcgaaccccctaggaagataataccagagaagaattcacttagacagacatacaacagctgtgccagactctgcttaaaccaagaaacagtatgtttagcaagcactgctatgaagactgagaattgtgtggccaaaacaaaacttgccaatggcacttccagtatgattgtgcccaagcaacggaaactctcagcaagctatgaaaaggaaaaggaactgtgtgtcaaatactttgagcagtggtcagagtcagatcaagtggaatttgtggaacatcttatatcccaaatgtgtcattaccaacatgggcacataaactcgtatcttaaacctatgttgcagagagatttcataactgctctgccagctcggggattggatcatatcgctgagaacattctgtcatacctggatgccaaatcactatgtgctgctgaacttgtgtgcaaggaatggtaccgagtgacctctgatggcatgctgtggaagaagcttatcgagagaatggtcaggacagattctctgtggagaggcctggcagaacgaagaggatggggacagtatttattcaaaaacaaacctcctgacgggaatgctcctcccaactctttttatagagcactttatcctaaaattatacaagacattgagacaatagaatctaattggagatgtggaagacatagtttacagagaattcactgccgaagtgaaacaagcaaaggagtttactgtttacagtatgatgatcagaaaatagtaagcggccttcgagacaacacaatcaagatctgggataaaaacacattggaatgcaagcgaattctcacaggccatacaggttcagtcctctgtctccagtatgatgagagagtgatcataacaggatcatcggattccacggtcagagtgtgggatgtaaatacaggtgaaatgctaaacacgttgattcaccattgtgaagcagttctgcacttgcgtttcaataatggcatgatggtgacctgctccaaagatcgttccattgctgtatgggatatggcctccccaactgacattaccctccggagggtgctggtcggacaccgagctgctgtcaatgttgtagactttgatgacaagtacattgtttctgcatctggggatagaactataaaggtatggaacacaagtacttgtgaatttgtaaggaccttaaatggacacaaacgaggcattgcctgtttgcagtacagggacaggctggtagtgagtggctcatctgacaacactatcagattatgggacatagaatgtggtgcatgtttacgagtgttagaaggccatgaggaattggtgcgttgtattcgatttgataacaagaggatagtcagtggggcctatgatggaaaaattaaagtgtgggatcttgtggctgctttggacccccgtgctcctgcagggacactctgtctacggacccttgtggagcattccggaagagtttttcgactacagtttgatgaattccagattgtcagtagttcacatgatgacacaatcctcatctgggacttcctaaatgatccagctgcccaagctgaacccccccgttccccttctcgaacatacacctacatctccagataa

SEQ ID NO: 51 - DNA, 8694 bp, Homo sapiens
tgaggaatcaacagccgccatcttgtcgcggacccgaccggggcttcgagcgcgatctactcggccccgccggtcccgggccccacaaccgcccgcgctcgctcctctccctcgcagccggcagggcccccgacccccgtccgggccctcgccggcccggccgcccgtgcccggggctgttttcgcgagcaggtgaaaatggctgagaacttgctggacggaccgcccaaccccaaaagagccaaactcagctcgcccggtttctcggcgaatgacagcacagattttggatcattgtttgacttggaaaatgatcttcctgatgagctgatacccaatggaggagaattaggccttttaaacagtgggaaccttgttccagatgctgcttccaaacataaacaactgtcggagcttctacgaggaggcagcggctctagtatcaacccaggaataggaaatgtgagcgccagcagccccgtgcagcagggcctgggtggccaggctcaagggcagccgaacagtgctaacatggccagcctcagtgccatgggcaagagccctctgagccagggagattcttcagcccccagcctgcctaaacaggcagccagcacctctgggcccacccccgctgcctcccaagcactgaatccgcaagcacaaaagcaagtggggctggcgactagcagccctgccacgtcacagactggacctggtatctgcatgaatgctaactttaaccagacccacccaggcctcctcaatagtaactctggccatagcttaattaatcaggcttcacaagggcaggcgcaagtcatgaatggatctcttggggctgctggcagaggaaggggagctggaatgccgtaccctactccagccatgcagggcgcctcgagcagcgtgctggctgagaccctaacgcaggtttccccgcaaatgactggtcacgcgggactgaacaccgcacaggcaggaggcatggccaagatgggaataactgggaacacaagtccatttggacagccctttagtcaagctggagggcagccaatgggagccactggagtgaacccccagttagccagcaaacagagcatggtcaacagtttgcccaccttccctacagatatcaagaatacttcagtcaccaacgtgccaaatatgtctcagatgcaaacatcagtgggaattgtacccacacaagcaattgcaacaggccccactgcagatcctgaaaaacgcaaactgatacagcagcagctggttctactgcttcatgctcataagtgtcagagacgagagcaagcaaacggagaggttcgggcctgctcgctcccgcattgtcgaaccatgaaaaacgttttgaatcacatgacgcattgtcaggctgggaaagcctgccaagttgcccattgtgcatcttcacgacaaatcatctctcattggaagaactgcacacgacatgactgtcctgtttgcctccctttgaaaaatgccagtgacaagcgaaaccaacaaaccatcctggggtctccagctagtggaattcaaaacacaattggttctgttggcacagggcaacagaatgccacttctttaagtaacccaaatcccatagaccccagctccatgcagcgagcctatgctgctctcggactcccctacatgaaccagccccagacgcagctgcagcctcaggttcctggccagcaaccagcacagcctcaaacccaccagcagatgaggactctcaaccccctgggaaataatccaatgaacattccagcaggaggaataacaacagatcagcagcccccaaacttgatttcagaatcagctcttccgacttccctgggggccacaaacccactgatgaacgatggctccaactctggtaacattggaaccctcagcactataccaacagcagctcctccttctagcaccggtgtaaggaaaggctggcacgaacatgtcactcaggacctgcggagccatctagtgcataaactcgtccaagccatcttcccaacacctgatcccgcagctctaaaggatcgccgcatggaaaacctggtagcctatgctaagaaagtggaaggggacatgtacgagtctgccaacagcagggatgaatattatcacttattagcagagaaaatctacaagatacaaaaagaactagaagaaaaacggaggtcgcgtttacataaacaaggcatcttggggaaccagccagccttaccagccccgggggctcagccccctgtgattccacaggcacaacctgtgagacctccaaatggacccctgtccctgccagtgaatcgcatgcaagtttctcaagggatgaattcatttaaccccatgtccttggggaacgtccagttgccacaagcacccatgggacctcgtgcagcctccccaatgaaccactctgtccagatgaacagcatgggctcagtgccagggatggccatttctccttcccgaatgcctcagcctccgaacatgatgggtgcacacaccaacaacatgatggcccaggcgcccgctcagagccagtttctgccacagaaccagttcccgtcatccagcggggcgatgagtgtgggcatggggcagccgccagcccaaacaggcgtgtcacagggacaggtgcctggtgctgctcttcctaaccctctcaacatgctggggcctcaggccagccagctaccttgccctccagtgacacagtcaccactgcacccaacaccgcctcctgcttccacggctgctggcatgccatctctccagcacacgacaccacctgggatgactcctccccagccagcagctcccactcagccatcaactcctgtgtcgtcttccgggcagactcccaccccgactcctggctcagtgcccagtgctacccaaacccagagcacccctacagtccaggcagcagcccaggcccaggtgaccccgcagcctcaaaccccagttcagcccccgtctgtggctacccctcagtcatcgcagcaacagccgacgcctgtgcacgcccagcctcctggcacaccgctttcccaggcagcagccagcattgataacagagtccctaccccctcctcggtggccagcgcagaaaccaattcccagcagccaggacctgacgtacctgtgctggaaatgaagacggagacccaagcagaggacactgagcccgatcctggtgaatccaaaggggagcccaggtctgagatgatggaggaggatttgcaaggagcttcccaagttaaagaagaaacagacatagcagagcagaaatcagaaccaatggaagtggatgaaaagaaacctgaagtgaaagtagaagttaaagaggaagaagagagtagcagtaacggcacagcctctcagtcaacatctccttcgcagccgcgcaaaaaaatctttaaaccagaggagttacgccaggccctcatgccaaccctagaagcactgtatcgacaggacccagagtcattacctttccggcagcctgtagatccccagctcctcggaattccagactattttgacatcgtaaagaatcccatggacctctccaccatcaagcggaagctggacacagggcaataccaagagccctggcagtacgtggacgacgtctggctcatgttcaacaatgcctggctctataatcgcaagacatcccgagtctataagttttgcagtaagcttgcagaggtctttgagcaggaaattgaccctgtcatgcagtcccttggatattgctgtggacgcaagtatgagttttccccacagactttgtgctgctatgggaagcagctgtgtaccattcctcgcgatgctgcctactacagctatcagaataggtatcatttctgtgagaagtgtttcacagagatccagggcgagaatgtgaccctgggtgacgacccttcacagccccagacgacaatttcaaaggatcagtttgaaaagaagaaaaatgataccttagaccccgaacctttcgttgattgcaaggagtgtggccggaagatgcatcagatttgcgttctgcactatgacatcatttggccttcaggttttgtgtgcgacaactgcttgaagaaaactggcagacctcgaaaagaaaacaaattcagtgctaagaggctgcagaccacaagactgggaaaccacttggaagaccgagtgaacaaatttttgcggcgccagaatcaccctgaagccggggaggtttttgtccgagtggtggccagctcagacaagacggtggaggtcaagcccgggatgaagtcacggtttgtggattctggggaaatgtctgaatctttcccatatcgaaccaaagctctgtttgcttttgaggaaattgacggcgtggatgtctgcttttttggaatgcacgtccaagaatacggctctgattgcccccctccaaacacgaggcgtgtgtacatttcttatctggatagtattcatttcttccggccacgttgcctccgcacagccgtttaccatgagatccttattggatatttagagtatgtgaagaaattagggtatgtgacagggcacatctgggcctgtcctccaagtgaaggagatgattacatcttccattgccacccacctgatcaaaaaatacccaagccaaaacgactgcaggagtggtacaaaaagatgctggacaaggcgtttgcagagcggatcatccatgactacaaggatattttcaaacaagcaactgaagacaggctcaccagtgccaaggaactgccctattttgaaggtgatttctggcccaatgtgttagaagagagcattaaggaactagaacaagaagaagaggagaggaaaaaggaagagagcactgcagccagtgaaaccactgagggcagtcagggcgacagcaagaatgccaagaagaagaacaacaagaaaaccaacaagaacaaaagcagcatcagccgcgccaacaagaagaagcccagcatgcccaacgtgtccaatgacctgtcccagaagctgtatgccaccatggagaagcacaaggaggtcttcttcgtgatccacctgcacgctgggcctgtcatcaacaccctgccccccatcgtcgaccccgaccccctgctcagctgtgacctcatggatgggcgcgacgccttcctcaccctcgccagagacaagcactgggagttctcctccttgcgccgctccaagtggtccacgctctgcatgctggtggagctgcacacccagggccaggaccgctttgtctacacctgcaacgagtgcaagcaccacgtggagacgcgctggcactgcactgtgtgcgaggactacgacctctgcatcaactgctataacacgaagagccatgcccataagatggtgaagtgggggctgggcctggatgacgagggcagcagccagggcgagccacagtcaaagagcccccaggagtcacgccgggtgagcatccagcgctgcatccagtcgctggtgcacgcgtgccagtgccgcaacgccaactgctcgctgccatcctgccagaagatgaagcgggtggtgcagcacaccaagggctgcaaacgcaagaccaacgggggctgcccggtgtgcaagcagctcatcgccctctgctgctaccacgccaagcactgccaagaaaacaaatgccccgtgcccttctgcctcaacatcaaacacaagctccgccagcagcagatccagcaccgcctgcagcaggcccagctcatgcgccggcggatggccaccatgaacacccgcaacgtgcctcagcagagtctgccttctcctacctcagcaccgcccgggacccccacacagcagcccagcacaccccagacgccgcagccccctgcccagccccaaccctcacccgtgagcatgtcaccagctggcttccccagcgtggcccggactcagccccccaccacggtgtccacagggaagcctaccagccaggtgccggcccccccacccccggcccagccccctcctgcagcggtggaagcggctcggcagatcgagcgtgaggcccagcagcagcagcacctgtaccgggtgaacatcaacaacagcatgcccccaggacgcacgggcatggggaccccggggagccagatggcccccgtgagcctgaatgtgccccgacccaaccaggtgagcgggcccgtcatgcccagcatgcctcccgggcagtggcagcaggcgccccttccccagcagcagcccatgccaggcttgcccaggcctgtgatatccatgcaggcccaggcggccgtggctgggccccggatgcccagcgtgcagccacccaggagcatctcacccagcgctctgcaagacctgctgcggaccctgaagtcgcccagctcccctcagcagcaacagcaggtgctgaacattctcaaatcaaacccgcagctaatggcagctttcatcaaacagcgcacagccaagtacgtggccaatcagcccggcatgcagccccagcctggcctccagtcccagcccggcatgcaaccccagcctggcatgcaccagcagcccagcctgcagaacctgaatgccatgcaggctggcgtgccgcggcccggtgtgcctccacagcagcaggcgatgggaggcctgaacccccagggccaggccttgaacatcatgaacccaggacacaaccccaacatggcgagtatgaatccacagtaccgagaaatgttacggaggcagctgctgcagcagcagcagcaacagcagcagcaacaacagcagcaacagcagcagcagcaagggagtgccggcatggctgggggcatggcggggcacggccagttccagcagcctcaaggacccggaggctacccaccggccatgcagcagcagcagcgcatgcagcagcatctccccctccagggcagctccatgggccagatggcggctcagatgggacagcttggccagatggggcagccggggctgggggcagacagcacccccaacatccagcaagccctgcagcagcggattctgcagcaacagcagatgaagcagcagattgggtccccaggccagccgaaccccatgagcccccagcaacacatgctctcaggacagccacaggcctcgcatctccctggccagcagatcgccacgtcccttagtaaccaggtgcggtctccagcccctgtccagtctccacggccccagtcccagcctccacattccagcccgtcaccacggatacagccccagccttcgccacaccacgtctcaccccagactggttccccccaccccggactcgcagtcaccatggccagctccatagatcagggacacttggggaaccccgaacagagtgcaatgctcccccagctgaacacccccagcaggagtgcgctgtccagcgaactgtccctggtcggggacaccacgggggacacgctagagaagtttgtggagggcttgtagcattgtgagagcatcaccttttccctttcatgttcttggaccttttgtactgaaaatccaggcatctaggttctttttattcctagatggaactgcgacttccgagccatggaagggtggattgatgtttaaagaaacaatacaaagaatatatttttttgttaaaaaccagttgatttaaatatctggtctctctctttggtttttttttggcgggggggtggggggggttcttttttttccgttttgtttttgtttggggggaggggggttttgtttggattctttttgtcgtcattgctggtgactcatgcctttttttaacgggaaaaacaagttcattatattcatattttttatttgtattttcaagactttaaacatttatgtttaaaagtaagaagaaaaataatattcagaactgattcctgaaataatgcaagcttataatgtatcccgataactttgtgatgtttcgggaagatttttttctatagtgaactctgtgggcgtctcccagtattaccctggatgataggaattgactccggcgtgcacacacgtacacacccacacacatctatctatacataatggctgaagccaaacttgtcttgcagatgtagaaattgttgctttgtttctctgataaaactggttttagacaaaaaatagggatgatcactcttagaccatgctaatgttactagagaagaagccttcttttctttcttctatgtgaaacttgaaatgaggaaaagcaattctagtgtaaatcatgcaagcgctctaattcctataaatacgaaactcgagaagattcaatcactgtatagaatggtaaaataccaactcatttcttatatcatattgttaaataaactgtgtgcaacagacaaaaagggtggtccttcttgaattcatgtacatggtattaacacttagtgttcggggttttttgttatgaaaatgctgttttcaacattgtatttggactatgcatgtgttttttccccattgtatataaagtaccgcttaaaattgatataaattactgaggtttttaacatgtattctgttctttaagatcccctgtaagaatgtttaaggtttttatttatttatatatattttttggtctgttctttgtaaaaaaaaaaaaaaaaa

SEQ ID NO: 52 - DNA, 2109 bp, Homo sapiens
cgcgcagcgcgccggagtggtcggggcccgcggccgctcgcgcctctcgatgggcagctcgcacttgctcaacaagggcctgccgcttggcgtccgacctccgatcatgaacgggcccctgcacccgcggcccctggtggcattgctggatggccgggactgcacagtggagatgcccatcctgaaggacgtggccactgtggccttctgcgacgcgcagtccacgcaggagatccatgagaaggtcctgaacgaggctgtgggggccctgatgtaccacaccatcactctcaccagggaggacctggagaagttcaaagccctccgcatcatcgtccggattggcagtggttttgacaacatcgacatcaagtcggccggggatttaggcattgccgtctgcaacgtgcccgcggcgtctgtggaggagacggccgactcgacgctgtgccacatcctgaacctgtaccggcgggccacctggctgcaccaggcgctgcgggagggcacacgagtccagagcgtcgagcagatccgcgaggtggcgtccggcgctgccaggatccgcggggagaccttgggcatcatcggacttggtcgcgtggggcaggcagtggcgctgcgggccaaggccttcggcttcaacgtgctcttctacgacccttacttgtcggatggcgtggagcgggcgctggggctgcagcgtgtcagcaccctgcaggacctgctcttccacagcgactgcgtgaccctgcactgcggcctcaacgagcacaaccaccacctcatcaacgacttcaccgtcaagcagatgagacaaggggccttcctggtgaacacagcccggggtggcctggtggatgagaaggcgctggcccaggccctgaaggagggccggatccgcggcgcggccctggatgtgcacgagtcggaacccttcagctttagccagggccctctgaaggatgcacccaacctcatctgcaccccccatgctgcatggtacagcgagcaggcatccatcgagatgcgagaggaggcggcacgggagatccgcagagccatcacaggccggatcccagacagcctgaagaactgtgtcaacaaggaccatctgacagccgccacccactgggccagcatggaccccgccgtcgtgcaccctgagctcaatggggctgcctataggtaccctccgggcgtggtgggcgtggcccccactggcatcccagctgctgtggaaggtatcgtccccagcgccatgtccctgtcccacggcctgccccctgtggcccacccgccccacgccccttctcctggccaaaccgtcaagcccgaggcggatagagaccacgccagtgaccagttgtagcccgggaggagctctccagcctcggcgcctggggcagcgggcccggaaaccctcgaccagagtgtgtgagagcatgtgtgtggtggcccctggcactgcagagactggtccgggctgtcaggagggcgggagggcgcagcgctgggcctcgtgtcgcttgtcgtccgtcctgtgggcgctctgccctgtgtccttcgcgttcctcgttaagcagaagaagtcagtagttattctcccatgaacgttcttgtctgtgtacagtttttagaacattacaaaggatctgtttgcttagctgtcaacaaaaagaaaacctgaaggagcatttggaagtcaatttgaggtttttttttttggtttttttttttttgtattttggaacgtgccccagaatgaggcagttggcaaacttctcaggacaatgaatcttcccgtttttctttttatgccacacagtgcattgttttttctacctgcttgtcttatttttagcataatttagaaaaacaaaacaaaggctgtttttcctaattttggcatgaacccccccttgttccaaaatgaagacggcatcatcacgaagcagctccaaaaggaaaagcttggcaggtgccctcgtcctggggacgtggagggtggcacggtccccgcctgcaccagtgccgtcctgctgatgtggtaggctagcaatattttggttaaaatcatgtttgtggccgaacgggcccctgcacccg

SEQ ID NO: 53 - DNA, 2636 bp, Homo sapiens
agcaccataacatggtgtgggaagtgaagacaaatcagatgcctaatgcagtacagaaactcctgttggtgatggacaagagagcctcaggaatgaatgactcattggagttgctgcagtgtaatgagaatttgccatcttcacctggatataactcctgtgatgaacacatggagcttgatgaccttcctgaacttcaggcagttcaaagtgatcctacccaatctggcatgtaccagctgagttcagatgtttcacatcaagaatacccaagatcatcttggaaccaaaatacctcagacataccagaaactacttaccgtgaaaatgaggtggactggctaacagaattggcaaatatcgcgaccagtccacaaagtccactgatgcagtgctcattttacaatagatcatctcctgtacacatcatagccactagcaaaagtttacattcctatgcacgccctccaccagtgtcctcttcttcgaagagtgaaccagccttccctcatcaccattggaaggaggaaacaccagtaagacacgaaagggcaaatagtgagtcagaatctggcattttctgcatgtcctccctgtcagatgatgatgatttgggatggtgcaattcctggccttcaactgtctggcactgttttttgaaaggcacacgactgtgctttcataagggaagcaataaggaatggcaagatgttgaagattttgctagagctgaaggctgtgataatgaggaagatcttcaaatgggcattcacaagggctatggttctgatggtctaaagttgttatcacatgaagaaagtgtatcatttggcgagtctgtactgaagttgacttttgatcctggtacagtagaagatggtttacttaccgtagagtgtaagctggaccaccctttctatgttaaaaataaaggttggtcatcattttatccaagcttgactgtggtacagcatggcattccatgttgtgaagttcatattggcgatgtatgtctacctcctggacaccccgatgccattaattttgatgattcaggtgtttttgatacatttaaaagctatgacttcacacctatggattcttctgcagtttatgtgttaagtagtatggctcgccagcgtcgtgcatctttgtcttgtggaggacctggtggtcaagactttgcaagatctggattcagtaaaaactgtggctcacctggatcatcacagctctcttccaattctttgtatgctaaagctgtcaaaaaccacagctcagggactgtgagtgccacttctcctaataagtgcaaaagaccaatgaatgccttcatgctttttgccaaaaaatacagagttgaatatactcagatgtatccagggaaagataacagagccataagtgtgatccttggtgacaggtggaagaaaatgaagaatgaagagagaagaatgtacacattagaagcaaaggctttggctgaagaacagaaacgtttaaatcctgactgttggaagaggaaaagaaccaattcaggctcacaacaacattaaaccaggatgcttatgttcttaagtctatatttgcatatacattgactcttgatggaaagacttaagaagatcaaggtctcaccatttgtcctcaattcgtgtgaccataagatactgatagcattgagtcttgaaatgatttaataatatgagtgaggatttgctttctccattagagcattaagctaaaactatcaacattttaaaccaaattgccttatttttcttccaaacttcatatatgtctatcaggtaataataggcttgaaaattgatatcctgtggtgctaaagtacagtagaaagagaggagaagtgtatacatgttttattttaaattgtacgaaaggggaatttaaaaaatatgtaactgctgtttatacattggctccttactgcttattaatctgtattgtacacatgatgaaatgaagcagaagctgggagtcggcctttcctctagtaaccaccacatggctcagcatctgtgccaaacataggcgctcctagtctggtcagtgccaagaggctaccagaacatggggcaggtggctggtgttggtgtcccagcctaagagccacctgctgcagttaccatggcatgctgagttgatgcaccaggtggcagcagccatccgttattatttccaatggagacctagcccaggccaaggtaaagttagttaatagcattgggatatagtcactgtaatggtgctattaacaaacagtcaacaccattgtattttttaacttcgtgttctgtatctcctcagccatgtatcttaaatatattttgtcatcataatctttatggtgggggcagactttgcacttactgcagtgcaacacttgcactttaattttcctccaactgtctaaaattagagcaaatacattggcaatacagctgcttttgctctgagctacaatcatggcttttcatgttacttaccaagtggtgtttctggttaggaatcacagctgtaaaattgatttcagttcatcacacttcttcatgatgttgcccctaaattttgcacactatattcttgtatattatttcaaataaatggaaaaa

SEQ ID NO: 54 - DNA, 3084 bp, Homo sapiens
aagatctaaaaacggacatctccaccgtgggtggctcctttttctttttctttttttcccacccttcaggaagtggacgtttcgttatcttctgatccttgcaccttcttttggggaaacggggcccttctgcccagatcccctctcttttctcggaaaacaaactactaagtcggcatccggggtaactacagtggagagggtttccgcggagacgcgccgccggaccctcctctgcactttggggaggcgtgctccctccagaaccggcgttctccgcgcgcaaatcccggcgacgcggggtcgcggggtggccgccggggcagcctcgtctagcgcgcgccgcgcagacgcccccggagtcgccagctaccgcagccctcgccgcccagtgcccttcggcctcggggcgggcgcctgcgtcggtctccgcgaagcgggaaagcgcggcggccgccgggattcgggcgccgcggcagctgctccggctgccggccggcggccccgcgctcgcccgccccgcttccgcccgctgtcctgctgcacgaacccttccaactctcctttcctcccccacccttgagttacccctctgtctttcctgctgttgcgcgggtgctcccacagcggagcggagattacagagccgccgggatgccccaactctccggaggaggtggcggcggcgggggggacccggaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctgaaatcatcccggccagcaacggacacgaggtggccagacaagcacaaacctctcaggagccctaccacgacaaggccagagaacaccccgatgacggaaagcatccagatggaggcctctacaacaagggaccctcctactcgagttattccgggtacataatgatgccaaatatgaataacgacccatacatgtcaaatggatctctttctccacccatcccgagaacatcaaataaagtgcccgtggtgcagccatcccatgcggtccatcctctcacccccctcatcacttacagtgacgagcacttttctccaggatcacacccgtcacacatcccatcagatgtcaactccaaacaaggcatgtccagacatcctccagctcctgatatccctactttttatcccttgtctccgggtggtgttggacagatcaccccacctcttggctggcaaggtcagcctgtatatcccatcacgggtggattcaggcaaccctacccatcctcactgtcagtcgacacttccatgtccaggttttcccatcatatgattcccggtcctcctggtccccacacaactggcatccctcatccagctattgtaacacctcaggtcaaacaggaacatccccacactgacagtgacctaatgcacgtgaagcctcagcatgaacagagaaaggagcaggagccaaaaagacctcacattaagaagcctctgaatgcttttatgttatacatgaaagaaatgagagcgaatgtcgttgctgagtgtactctaaaagaaagtgcagctatcaaccagattcttggcagaaggtggcatgccctctcccgtgaagagcaggctaaatattatgaattagcacggaaagaaagacagctacatatgcagctttatccaggctggtctgcaagagacaattatggtaagaaaaagaagaggaagagagagaaactacaggaatctgcatcaggtacaggtccaagaatgacagctgcctacatctgaaacatggtggaaaacgaagctcattcccaacgtgcaaagccaaggcagcgaccccaggacctcttctggagatggaagcttgttgaaaacccagactgtctccacggcctgcccagtcgacgccaaaggaacactgacatcaattttaccctgaggtcactgctagagacgctgatccataaagacaatcactgccaacccctctttcgtctactgcaagagccaagttccaaaataaagcataaaaaggttttttaaaaggaaatgtaaaagcacatgagaatgctagcaggctgtggggcagctgagcagcttttctccccccatatctgcgtgcacttcccagagcatcttgcatccaaacctgtaacctttcggcaaggacggtaacttggctgcatttgcctgtcatgcgcaactggagccagcaaccagctatccatcagcaccccagtggaggagttcatggaagagttccctctttgtttctgcttcatttttctttcttttcttttctcctaaagcttttatttaacagtgcaaaaggatcgtttttttttgcttttttaaacttgaatttttttaatttacactttttagttttaattttcttgtatattttgctagctatgagcttttaaataaaattgaaagttctggaaaagtttgaaataatgacataaaaagaagccttctttttctgagacagcttgtctggtaagtggcttctctgtgaattgcctgtaacacatagtggcttctccgcccttgtaaggtgttcagtagagctaaataaatgtaatagccaaaccccactctgttggtagcaattggcagccctatttcagtttattttttcttctgttttcttcttttctttttttaaacagtaaaccttaacagatgcgttcagcagactggtttgcagtgaattttcatttctttccttatcacccccttgttgtaaaaagcccagcacttgaattgttattactttaaatgttctgtatttgtatctgtttttattagccaattagtgggattttatgccagttgttaaaatgagcattgatgtacccattttttaaaaaagcaagcacagcctttgcccaaaactgtcatcctaacgtttgtcattccagtttgagttaatgtgctgagcatttttttaaaagaagctttgtaataaaacatttttaaaaattgtcatttaaaaaaaaaaaaaaaaaa

SEQ ID NO: 55 - DNA, 2727 bp, Homo sapiens
tttcccttttttttccgtttggcaacccacacccttccacacacgctcacccgaaaattaaacaccaagatcctctaacttgtttggattgactgatgaagacataaagctctatgttttttgaggtggagtgagtggtttttcttcatttttaaatggccaaatgacagcttgacccagtttgctttccaatcaaagggcatttattttgaatgtctctttgtggcgcaagagccaacgcaaaaatgatggcggcttacaatggcggtacatctgcagcagcagcaggtcaccaccaccaccatcaccaccaccttccacacctccctcctcctcacctgcaccaccaccaccaccctcaacaccatcttcatccggggtcggctgccgctgtacaccctgtacagcagcacacctcttcggcagctgcggcagccgcagcagcggctgcagctgcagccatgttaaaccctgggcaacaacagccatatttcccatcaccggcaccggggcaggctcctggaccagctgcagcagccccagctcaggtacaggctgccgcagctgctacagttaaggcgcaccatcatcagcactcgcatcatccacagcagcagctggatattgagccggatagacctattggatatggagcctttggtgttgtctggtcagtaacagatccaagagatggaaagagagtagcgctcaaaaagatgcccaacgtcttccagaatctggtctcttgcaaaagggtcttccgggaattgaagatgttgtgtttttttaagcatgataatgtactctctgcccttgacatactccaacctccacacattgactattttgaagaaatatatgttgtcacagaattgatgcagagtgacctacataaaattatcgtctctcctcaaccactcagctcagatcatgtcaaagtttttctttatcagattttgcgaggtttgaaatatctccattcagctggcattttacatcgagacattaagccagggaatctccttgtgaacagcaactgtgttctaaagatttgtgattttggattggccagagtggaagaattagatgaatcccgtcatatgactcaggaagttgttactcagtattatcgggctccagaaatcctgatgggcagccgtcattacagcaatgctattgacatctggtctgtgggatgtatctttgcagaactactaggacgaagaatattgtttcaggcacagagtcccattcagcagttggatttgatcacggatctgttgggcacaccatcactggaagcaatgaggacagcttgtgaaggcgctaaggcacatatactcaggggtcctcataaacagccatctcttcctgtactctataccctgtctagccaggctacacatgaagctgttcatctcctttgcaggatgttggtctttgatccatccaaaagaatatccgctaaggatgccttagcccacccctacctagatgaagggcgactacgatatcacacatgtatgtgtaaatgttgcttttccacctccactggaagagtttataccagtgactttgagcctgtcaccaatcccaaatttgatgacactttcgagaagaacctcagttctgtccgacaggttaaagaaattattcatcagttcattttggaacagcagaaaggaaacagagtgcctctctgcatcaaccctcagtctgctgcttttaagagctttattagttccactgttgctcagccatctgagatgcccccatctcctctggtgtgggagtgatggtggaagataatgtactactgaagatgtaatgtagctttccactggagtctgggatttgcaattctggaggttaatcatgcttgtactgtaattttactaatgaagttttaaattaacaaccactacttgtatgatatgaataatatttagaaatgttactagacttttaatcttgtaaagtggttgtgcttttagaagaaaaatattttacccagagttgcacatgttttatgaatttagtgcagctgttatggctcacctcagaacaaaagagaattgaaccaaatttgggagtttggggttttatgttttgtttttcttttctaaaatgaagtgagattgttcacacacacacacacacacacacacacacacaaacacaaaggacagtcatacattttgatatttgagccattcctaaagatttggggttttctaaaactaaagaatctaggaaccttgcctgcgaccaatcatggagccacgtgagctgatcgtggctgcacctggggggagggtagggaggaggggcatgccacctaatgatcaagccctataattagcttctcattagagccgtgatggtgatgtgtgctgtctaaaatccaatgttgtgggtagagagaatgagtttgtgactaggagagactaaacttttgttttccttacccagtataaatatatatatatatatttaatctatttttattagaagtttttctgctctttcttacataaaagaaccccaagcatgcatctttcatgtgtgtaaataattcatttctgggctaatttcaaaagaatcccaatattgctgtatagaaagagaactagcttgcacattttaggtctgtgaaattttgtgagacttttcctgcactggacagtaaaaaaataataaaagacaaaaacaaatttaaaaaaaaaa

SEQ ID NO: 56 - DNA, 1518 bp, Homo sapiens
gcggccgcgtcgacgagcatcatggcaaccgttacagccacaaccaaagtcccggagatccgtgatgtaacaaggattgagcgaatcggtgcccactcccacatccggggactggggctggacgatgccttggagcctcggcaggcttcgcaaggcatggtgggtcagctggcggcacggcgggcggctggcgtggtgctggagatgatccgggaagggaagattgccggtcgggcagtccttattgctggccagccgggcacggggaagacggccatcgccatgggcatggcgcaggccctgggccctgacacgccattcacagccatcgccggcagtgaaatcttctccctggagatgagcaagaccgaggcgctgacgcaggccttccggcggtccatcggcgttcgcatcaaggaggagacggagatcatcgaaggggaggtggtggagatccagattgatcgaccagcaacagggacgggctccaaggtgggcaaactgaccctcaagaccacagagatggagaccatctacgacctgggcaccaagatgattgagtccctgaccaaggacaaggtccaggccggggacgtgatcaccatcgacaaggcgacgggcaagatctccaagctgggccgctccttcacacgcgcccgcgactacgacgctatgggctcccagaccaagttcgtgcagtgcccagatggggagctccagaaacgcaaggaggtggtgcacaccgtgtccctgcacgagatcgacgtcatcaactctcgcacccagggcttcctggcgctcttctcaggtgacacaggggagatcaagtcagaagtccgtgagcagatcaatgccaaggtggctgagtggcgcgaggagggcaaggcggagatcatccctggagtgctgttcatcgacgaggtccacatgctggacatcgagagcttctccttcctcaaccgggccctggagagtgacatggcgcctgtcctgatcatggccaccaaccgtggcatcacgcgaatccggggcaccagctaccagagccctcacggcatccccatagacctgctggaccggctgcttatcgtctccaccaccccctacagcgagaaagacacgaagcagatcctccgcatccggtgcgaggaagaagatgtggagatgagtgaggacgcctacacggtgctgacccgcatcgggctggagacgtcactgcgctacgccatccagctcatcacagctgccagcttggtgtgccggaaacgcaagggtacagaagtgcaggtggatgacatcaagcgggtctactcactcttcctggacgagtcccgctccacgcagtacatgaaggagtaccaggacgccttcctcttcaacgaactcaaaggcgagaccatggacacctcctgagttggatgtcatcccccgaccccaccctgttttccaccagagttctgacactgtgactctgtataaaatggttgggaagctgcaaaaaaaaaaaaaaaaaaaaaa

SEQ ID NO: 57 - DNA, 932 bp, Homo sapiens
gggcgtgagaaaggcgacggcggcggcgcggaggagggttatctatacatttaaaaaccagccgcctgcgccgcgcctgcggagacctgggagagtccggccgcacgcgcgggacacgagcgtcccacgctccctggcgcgtacggcctgccaccactaggcctcctatccccgggctccagacgacctaggacgcgtgccctggggagttgcctggcggcgccgtgccagaagcccccttggggcgccacagttttccccgtcgcctccggttcctctgcctgcaccttcctgcggcgcgccgggacctggagcgggcgggtggatgcaggcgcgatggacggcggcacactgcccaggtccgcgccccctgcgccccccgtccctgtcggctgcgctgcccggcggagacccgcgtccccggaactgttgcgctgcagccggcggcggcgaccggccaccgcagagaccggaggcggcgcagcggccgtagcgcggcgcaatgagcgcgagcgcaaccgcgtgaagctggtgaacttgggcttccaggcgctgcggcagcacgtgccgcacggcggcgccagcaagaagctgagcaaggtggagacgctgcgctcagccgtggagtacatccgcgcgctgcagcgcctgctggccgagcacgacgccgtgcgcaacgcgctggcgggagggctgaggccgcaggccgtgcggccgtctgcgccccgcgggccgccagggaccaccccggtcgccgcctcgccctcccgcgcttcttcgtccccgggccgcgggggcagctcggagcccggctccccgcgttccgcctactcgtcggacgacagcggctgcgaaggcgcgctgagtcctgcggagcgcgagctactcgacttctccagctggttagggggctactgagcgccctcgaccta

SEQ ID NO: 58 - DNA, 5182 bp, Homo sapiens
tctcttcccctctccccttctcttccccgaggctatgtccacccggtgcggcgaggcgggcagagccagaggcacgcagccgcacaggggctacagagcccagaatcagccctacaagatgcacttaggacccccgcggctggaagaatgagcttgtccttcctcctcctcctcttcttcagccacctgatcctcagcgcctgggctcacggggagaagcgtctcgcccccaaagggcaacccggacccgctgccactgataggaaccctagaggctccagcagcagacagagcagcagtagcgctatgtcttcctcttctgcctcctcctcccccgcagcttctctgggcagccaaggaagtggcttggagcagagcagtttccagtggagcccctcggggcgccggaccggcagcctctactgcagagtgggcatcggtttccatctgcagatctacccggatggcaaagtcaatggatcccacgaagccaatatgttaagccaagttcacagatgactgcaagttcagggagcgttttcaagaaaatagctataatacctatgcctcagcaatacatagaactgaaaaaacagggcgggagtggtatgtggccctgaataaaagaggaaaagccaaacgagggtgcagcccccgggttaaaccccagcatatctctacccattttctgccaagattcaagcagtcggagcagccagaactttctttcacggttactgttcctgaaaagaaaaagccacctagccctatcaagccaaagattcccctttctgcacctcggaaaaataccaactcagtgaaatacagactcaagtttcgctttggataatattcctcttggccttgtgagaaaccattctttcccctcaggagtttctataggtgtcttcagagttctgaagaaaaattactggacacagcttcagctatacttacactgtattgaagtcacgtcatttgtttcaatgtgactgaaacaaaatgttttttgataggaaggaaactggaattctttgtactaatacagggagcacactccttcagttcagcaagacataaagccttttgctttatgcttgagggatatttagaactttgtattttcggaaagttaaataacagggactacgtatttttctgacttttacagattaacctgaaagaacatacatgatacatttttatttttggtttccaaagaatattttgatgcagataaaatattttgttaacttttgtttttttttgtttgttttcttaaaagtacctctgcattgagcatattttcttacttttattattttaattaatatgacataagcaatcattttatgctgtttatgaattataaatgtgtttatagctcatttgtaatatggaaatcttttacatttttcctattcactgcacttttttattgtttttatttctagccatacctcagataatatgtttagttttacattttaaaatgtttaaattctctttcacagcaccaaaggctcagcttggatttgtgtgtatgtgtatgtcaattcatgacattatgtggaatcctaaacctttggtggctgggatatgatgggttagaagcaaggagaaaatataaggactttttgatggaattaaatgtgggaggtaaggaaaaggatttagaggtaaaagtacactaagtttgcaacatttattgagatctaagtctgtcttgccttcatttctctttttatctcccccttgccctcattcttgaacagctggaggaatacattttattctgtccatgaagcatacactatgaaattcaagtgcttaaaaatacttctatgactctctgctatcccactgtatagatccacagggagcaaacacttagaaatgatagagaactgaaggagatcaatggtttaacagttatccatgccaagtcccattgtcagaaatattcttattactcagtcaaacactctttgagcttcccttcctaaaggtaaccaatccagtgaatagatgtgcccttttataaggaaacttctgatgtttattaaaaaaactggccttttgatagaggtaacttaatttgggaatttgttgtgttgaaatggcatttaatttcaacctaaatactgactgctggacataaatcacagaaaatttaacttaagaaaatttacaaaatttattctcaggtaatcattttaataaagttctgcaaaatacacgtttatcttacattcagaaatgtggcaaaaaaggcatagctaaaggctaaacatatggctttagtagtaacaaaagggttcatagaaacttcatggtttgcatttaaacatgtttaaagtgtacttataaactatttttttcttaaagcaaactatgatttattttggtgcacaaatacaaagtggaaacttaccaaaattgaactagctaccatataagcagattgctttaatttgatgggaaaatagtacacacatatatataacaaataatatattaaaaaacccatccatcaactaaaacattatatgtatacatcagtatagtgttttattataaagccaattatctgattaagcattctttccactgaatgcataatgtttaaatagcataaaatgaaatgctacaaaaattgaactaatttatactttaaagtatttctgggttaaatgaaacaatgaaattttttagtatgttcaactctcatccaaatggcatatgaccctgtttacacagcctaaagctaaaaatattactctagtttattctaatctattgttaagtattgtgcactgtataccaagttcttagggcacatgaaaaattttagctgccaaacaggaactagtaaacatatgttcctaataagtgaagggaaagataataatgatggtcaacaataagccacgtcaatgcataagttgtataggctaaatgttgcttgtaggctacattaaactcaaatgtaatagtttatcttatactcctggtttgatttgattagcatattaacgtgaaagtaggatagctactaaatatatattatgcaagtcaggaatcattaatttcaaaatttaaagccatgctaaaattaaaaagaaaatattaaattacacaattacacttgtctttactggccatacaaaatgattttttttttttttttgagacagagtcttgctctgtcaccaggctggagtgcagtggcatgatctcggctcactgcaacctccaactccctggtttaagggattctcctgcctcagcctcccaagtagctgggattacagactcatgccaccacgccagctaatttttgtatttttagtagagacggggtttcaccatgttggtcaggatggtctcaatcctggcctcttgatagtcctgacctcatgatctgcccacctcggcctccccaaagtgctgggattacaggtacaatgatgtataattaatgcttagtgaagcataaagttacctacatcaattaattaaatgaacttatgtacagaaaacatgtataaatataagtctatactaatgcttacaactttctaagagggttcttgcttatgtagctttttattattttaagtaactagaaccaccaaatatcaaataaaattatttggttatggttatgttcatctaaacacaacaataacttttatattaatatttaggagtctattttgtctataggtgacaaacatctccagactaacatgtcagttttatcaattatattatgtttaattatttaagatttctttatgtggaacatctatagagataaatagaaattttcaataagatgtagtaacactgtgatttatctttcaagagtctctcttcacttccttctaaagagactaatttgagagtacaggtgcatattaattttcttggttctttcagctgaattatattggtccagaagttcaaaatcatgtgacaataataagggatactgacagaagttatttccaagtttgtgtatatattataaaaattacatatataaaactaaggcttttatttctgttatttttaagcttttatttcttgtagctaaaaataaaacatcataaatctggtaggtaaatttcttattaaatcaatcttgaaatagaaaatgtaataactttcttaccattaacattttttacccttccatagaagggagggaataaatcatgacttatcccattttcaataacaaaacgaaactatggcactaaccaaaaacttgcattctggcataatttttacagttgcagagaattgtttctgggctcattaaaaaaagtagtattgcagacattgctgcaatgggaagcagacaataacttcttaaaggaattctacacctcctttaagatttacttaattgctacatctaaattctgataatttaaaatccattttaggtgataaaattttttaaaagttttgaaggaaacctctggataaatggacaaggcctaatttttttttgtagtcaatccaactgtactggccaatttttgaaataagattatatgattaggtattagcagagacaaagagttacctcctccatcttactctgccctatttgaaagtctcaggggagaaaagggaacaagatgctgatccaacctgagtggagtcaggtgaggcatctttacatctaagaatttttttttaaattttattattattatacttcaagttctagggtacatgtccacaatgcacatgtctgtcacacatgcacacatgtgccatgctggtgtgctgcacccaccaacctgtcatccagcattaggtatatctcctaatgctatccctcccctctccacccaccccacagcaggccccggtatgtgatgttccccttcgtgtgtccatgtgttcttattgttcaattcccacctatgagtgagaatatgtggtgtttggtttttggtccttgcaatagtttgctgagaatgatggtttccagcttcatccatgtccctacaaagaacatgaactcatcatttttgatggctgcatagtattccatggtgtatatgtgccacattttcttaatccagtctatcattgttggacatttgggttggttccagtctttgctattgtgaatagtgctgcaataaacatatgtgtg

SEQ ID NO: 59 - DNA, 1873 bp, Homo sapiens
tgcgtgcccccaggcccagcgcgcctccgggcgagtccccaggagcgcggcccaatggatcgctccgggcccgccccctcgcgcgctgattggccgccgccccgctggcctcgccttattagcaagttctctggggagccgcggtagggcccggagccggcgagtgctcccgggaactctgcctgcgcggcggcagcgaccggaggccaggcccagcacgccggagctggcctgctggggaggggcgggaggcgcgcgcgggagggtccgcccggccagggccccgggcgctcgcagaggccggccgcgctcccagcccgcccggagcccatgcccggcggctggccagtgctgcggcagaagggggggcccggctctgcatggccccggctgctgacatgacttctttgccactcggtgtcaaagtggaggactccgccttcggcaagccggcggggggaggcgcgggccaggcccccagcgccgccgcggccacggcagccgccatgggcgcggacgaggagggggccaagcccaaagtgtccccttcgctcctgcccttcagcgtggaggcgctcatggccgaccacaggaagccgggggccaaggagagcgccctggcgccctccgagggcgtgcaggcggcgggtggctcggcgcagccactgggcgtcccgccggggtcgctgggagccccggacgcgccctcttcgccgcggccgctcggccatttctcggtggggggactcctcaagctgccagaagatgcgctcgtcaaagccgagagccccgagaagcccgagaggaccccgtggatgcagagcccccgcttctccccgccgccggccaggcggctgagccccccagcctgcaccctccgcaaacacaagacgaaccgtaagccgcggacgcccttcaccaccgcgcagctgctggcgctggagcgcaagttccgccagaagcagtacctgtccatcgccgagcgcgcggagttctccagctcgctcagcctcactgagacgcaggtgaagatatggttccagaaccgccgcgccaaggcaaagagactacaagaggcagagctggagaagctgaagatggccgccaagcccatgctgccaccggctgccttcggcctctccttccctctcggcggccccgcagctgtagcggccgcggcgggtgcctcgctctacggtgcctctggccccttccagcgcgccgcgctgcctgtggcgcccgtgggactctacacggcccatgtgggctacagcatgtaccacctgacatagagggtcccaggtcgcccacctgtgggccagccgattcctccagccctggtgctgtacccccggacgtgctcccctgctcggcaccgccagccgccttccctttaaccctcacactgctccagtttcacctctttgctccctgagttcactctccgaagtctgatccctgccaaaaagtggctggaagagtcccttagtactcttctagcatttagagatctaccctctcgagttaaaagatggggaaactgagggcagagaggttaacagatttatctaaggtccccagcagaattgacagtgtgaacagagctagaggccatgtctcctgcatagtttttccctgtcctgacaccaggcaagaaagcgcagagaaatcggtgtctgacgattttggaatgagaacaatctcaaaaaaaaaaaaaaaaaaaaaaaaaagaaaagagaaaaaaaagactagccaccaggaagatgaatcctagcttcttccattggaaaatttaaagacaagttcaacaacaaaacatttgctctggggggcagggaaaacacagatgtgttgcaaaggtaggttgaaggga

SEQ ID NO: 60 - DNA, 714 bp, Homo sapiens
atgccagcccgccttgagacctgcatctccgacctcgactgcgccagcagcagcggcagtgacctatccggcttcctcaccgacgaggaagactgtgccagactccaacaggcagcctccgcttcggggccgcccgcgccggcccgcaggagcgcgcccaatatctcccgggcgtctgaggttccaggggcacaggacgacgagcaggagaggcggcggcgccgcggccggacgcgggtccgctccgaggcgctgctgcactcgctgcgcaggagccggcgcgtcaaggccaacgatcgcgagcgcaaccgcatgcacaacttgaacgcggccctggacgcactgcgcagcgtgctgccctcgttccccgacgacaccaagctcaccaaaatcgagacgctgcgcttcgcctacaactacatctgggctctggccgagacactgcgcctggcggatcaagggctgcccggaggcggtgcccgggagcgcctcctgccgccgcagtgcgtcccctgcctgcccggtcccccaagccccgccagcgacgcggagtcctggggctcaggtgccgccgccgcctccccgctctctgaccccagtagcccagccgcctccgaagacttcacctaccgccccggcgaccctgttttctccttcccaagcctgcccaaagacttgctccacacaacgccctgtttcattccttaccactag

SEQ ID NO: 61 - DNA, 754 bp, Homo sapiens
ggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggagccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatctag

SEQ ID NO: 62 - DNA, 1167 bp, Homo sapiens
cctcggaccccattctctcttcttttctcctttggggctggggcaactcccaggcgggggcgcctgcagctcagctgaacttggcgaccagaagcccgctgagctccccacggccctcgctgctcatcgctctctattcttttgcgccggtagaaaggatgacgcctcaaccctcgggtgcgcccactgtccaagtgacccgtgagacggagcggtccttccccagagcctcggaagacgaagtgacctgccccacgtccgccccgcccagccccactcgcacacgggggaactgcgcagaggcggaagagggaggctgccgaggggccccgaggaagctccgggcacggcgcgggggacgcagccggcctaagagcgagttggcactgagcaagcagcgacggagtcggcgaaagaaggccaacgaccgcgagcgcaatcgaatgcacaacctcaactcggcactggacgccctgcgcggtgtcctgcccaccttcccagacgacgcgaagctcaccaagatcgagacgctgcgcttcgcccacaactacatctgggcgctgactcaaacgctgcgcatagcggaccacagcttgtacgcgctggagccgccggcgccgcactgcggggagctgggcagcccaggcggttcccccggggactgggggtccctctactccccagtctcccaggctggcagcctgagtcccgccgcgtcgctggaggagcgacccgggctgctgggggccaccttttccgcctgcttgagcccaggcagtctggctttctcagattttctgtgaaaggacctgtctgtcgctgggctgtgggtgctaagggtaagggagagggagggagccgggagccgtagagggtggccgacggcggcggccctcaaaagcacttgttccttctgcttctccctggctgacccctggccggcccaggcctccacgggggcggcaggctgggttcattccccggccctccgagccgcgccaacgcacgcaacccttgctgctgcccgcgcgaagtgggcattgcaaagtgcgctcattttaggcctcctctctgccaccaccccataatctcattcaaagaatactagaatggtagcactacccggccggagccgcccaccgtcttgggtcgccctaccctcactca

SEQ ID NO: 63 - DNA, 3160 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcaggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaaaggttttgctaccattctaatgcatgtattcgggttagggcaatggaggggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaaaaaaaaaa

SEQ ID NO: 64 - DNA, 1528 bp, Homo sapiens
gagggaagtgtcgactgcgccggcgggaacagacatgcctgcggccaagaagcagaagctgagcagtgacgagaacagcaatccagaactctctggagacgagaatgatgacgctgtcagtatagaaagtggtacaaacactgaacgccctgatacacctacaaacacgccaaatgcacctggaaggaaaagttggggaaagggaaaatggaagtcaaagaaatgcaaatattctttcaaatgtgtaaatagtctcaaggaagatcataaccaaccattgtttggagttcagtttaactggcacagtaaagaaggagatccattagtgtttgcaactgtaggaagcaacagagttaccttgtatgaatgtcattcacaaggagaaatccggttgttgcaatcttacgtggatgctgatgctgatgaaaacttttacacttgtgcatggacctatgatagcaatacgagccatcctctgctggctgtagctggatctagaggcataattaggataataaatcctataacaatgcagtgtataaagcactatgttggccatggaaatgctatcaatgagctgaaattccatccaagagatccaaatcttctcctgtcagtaagtaaagatcatgctttacgattatggaatatccagacggacactctggtggcaatatttggaggcgtagaagggcacagagatgaagttctaagtgctgattatgatcttttgggtgaaaaaataatgtcctgtggtatggatcattctcttaaactttggaggatcaattcaaagagaatgatgaatgcaattaaggaatcttatgattataatccaaataaaactaacaggccatttatttctcagaaaatccattttcctgatttttctaccagagacatacataggaattatgttgattgtgtgcgatggttaggcgatttgatactttctaagtcttgtgaaaatgccattgtgtgctggaaacctggcaagatggaagatgatatagataaaattaaacccagtgaatctaatgtgactattcttgggcgatttgattacagccagtgtgacatttggtacatgaggttttctatggatttctggcaaaagatgcttgcattgggcaatcaagttggcaaactttatgtttgggatttagaagtagaagatcctcataaagccaaatgtacaacactgactcatcataaatgtggtgctgctattcgacaaaccagttttagcagggatagcagcattcttatagctgtttgtgatgatgccagtatttggcgctgggatcgacttcgataaaatacttttgcctaatcaaaattaaaatgtgtttgttgtctgtgtaaaataaaattaatgtatcttgctagtaagggcacgtagagcatttagagttgtctttcagcattcaatcaggctgagctgaatgtagtgatgtttacattgtttacattctttgtactgtcttcctgctcagactctactgcttttaataaaaatttatttttg

SEQ ID NO: 65 - DNA, 2587 bp, Homo sapiens
  misc_feature (1751)..(1751): unknown nucleotide
agggcgaacgggcgagtggcagcgaggcggggcgggctgaggccagcgcggaagtctcgcgaggccgggcccgagcagagtgtggcggcggcggcgagatctgggctcgggttgaggagttggtatttgtgtggaaggaggcggaggcgcaggaggaggaagggggaagcggagcgcggcccggacgggaggaggcgcggccagggcgggcggttgcggcgaggcgaggcgaggagggggagccgagacgcagcggccgaggcgggggcgcgggcgcaccgaggcgagggaggcggggaagccccgccgccgccgcggcgcccgccccttcccccgccgcccgcgccctctccccccgccgcgctcgccgccttcctccctcgcttccttccccacggccggccgcctcctcgcccgcccgcccgcagccgaggagccgaggccgccggggccgtggcggcggagccctcagccatggcctcgggcgacaccctctacatcgccacggacggctcggagatgccggccgagatcgtggagctgcacgagatcgaggtggagaccatcccggtggagaccatcgagaccacagtggtgggcgaggaggaggaggaggacgacgacgacgaggacggcggcggtggcgaccacggcggcgggggcggccacgggcacgccggccaccaccaccaccaccatcaccaccaccaccacccgcccatgatcgctctgcagccgctggtcaccgacgacccgacccaggtgcaccaccaccaggaggtgatcctggtgcagacgcgcgaggaggtggtgggcggcgacgactcggacgggctgcgcgccgaggacggcttcgaggatcagattctcatcccggtgcccgcgccggccggcggcgacgacgactacattgaacaaacgctggtcaccgtggcggcggccggcaagagcggcggcggcggctcgtcgtcgtcgggaggcggccgcgtcaagaagggcggcggcaagaagagcggcaagaagagttacctcagcggcggggccggcgcggcgggcggcggcggcgccgacccgggcaacaagaagtgggagcagaagcaggtgcagatcaagaccctggagggcgagttctcggtcaccatgtggtcctcagatgaaaaaaaagatattgaccatgagacagtggttgaagaacagatcattggagagaactcacctcctgattattcagaatatatgacaggaaagaaacttcctcctggaggaatacctggcattgacctctcagatcccaaacaactggcagaatttgctagaatgaagccaagaaaaattaaagaagatgatgctccaagaacaatagcttgccctcataaaggctgcacaaagatgttcagggataactcggccatgagaaaacatctgcacacccacggtcccagagtccacgtctgtgcagaatgtggcaaagcttttgttgagagttcaaaactaaaacgacaccaactggttcatactggagagaagccctttcagtgcacgttcgaaggctgtgggaaacgcttttcactggacttcaatttgcgcacacatgtgcgaatccataccggagacaggccctatgtgtgccccttcgatggttgtaataagaagtttgctcagtcaactaacctgaaatctcacatcttaacacatgctaaggccaaaaacaaccagtgaaaagaagagagaagacccttctcgaccacggnaagcatcttccagaagtgtgattgggaataaatatgcctctcctttgtatattatttctaggaagaattttaaaaatgaatcctacacacctaagggacatgttttgataaagtagtaaaaaaaaaaaaaaaaaaactttactaagatgacattgctaagatgctctatcttgctctgtaatctcgtttcaaaaacacagtgtttttgtaaagtgtggtcccaacaggaggacaattcatgaacttcgcatcaaaagacaattctttatacaacagtgctaaaaatgggacttcttttcacattcttataaatatgaagctcacctgttgcttacaatttttttaattttgtattttccaagtgtgcatattgtacacttttttggggatatgcttagtaatgctacgtgtgatttttctggaggttgataactttgcttgcagtagattttctttaaaagaatgggcagttacatgcatacttcaaaagtattttcctgtaaaaaaaaaaaaagttatataggttttgtttgctatcttaattttggttgtattctttgatgttaacacattttgtataattgtatcgtatagctgtattgaatcatgtagtatcaaatattagatgtgatttaatagtgttaatcaatttaaacccattttagtcactttttttttccaaaaaaatactgccagatgctgatgttcagtgtaatttctttgcctgttcagttacagaaagtggtgctcagttgtagaatgtattgtaccttttaacacctgatgtgtacatcccatgtaacagaaagggcaacaataaaatagcaatcctaaag

SEQ ID NO: 66 - DNA, 356 bp, Homo sapiens
ctgccagagatgagtgcaggtgaggagagtagcagctcggactccgaccccgactccgcctcgccgcccagcactggacagaacccatcagtgtccgttcagaccagccaggactggaagcccacccgcagcctcatcgagcacgtatttgtcaccgacgtcactgccaacctcatcaccgtcacagtgaaggagtctcccaccagcgtcggcttcttcaacctgaggcattactgaagccccggcgccaccagctgcgcgtcttactccccttccctgcctatggtgtcgcttggctaagtgactcccagcccaagccccctcaagagtctgatcgtcgggggaggaggagtggg

SEQ ID NO: 67 - DNA, 1260 bp, Homo sapiens
attatgggctgtgggtgccgctgagcaagatggagctgtctgcagtgggcgagcgggtcttcgcggccgaatccatcatcaaacggcggatccgaaagggacgcatcgagtacctggtgaaatggaaggggtgggcgatcaagtacagcacttgggagcccgaggagaacatcctggactcgcggctcattgcagccttcgaacaaaaggagagggagcgtgagctgtatgggcccaagaagaggggacccaaacccaaaactttcctcctgaaggcgcgggcccaggccgaggccctccgcatcagtgatgtgcatttctctgtcaagccgagcgccagtgcctcctcgcccaagctgcactccagcgcagccgtgcaccggctcaagaaggacatccgccgctgccaccgtatgtcccgccgtcccctgccccgcccggacccgcaggggggcagccccggactgcgcccgcccatttcgcccttctcggagacggtgcgcatcatcaaccgcaaggtgaagccgcgggagcccaagcggaaccgcatcatcctgaacctgaaggtgatcgacaagggcgctggcggcgggggcgccgggcagggggccggggcgctggcccgccccaaagtcccctcgcggaaccgcgttataggcaagagcaagaagttcagcgagagcgtcctgcgtacacagatccgccacatgaagttcggcgcctttgcgctgtacaagcctccgcccgcccccctggtagccccgtcccccggcaaggctgaggcctcagccccgggccctgggctacttctggccgcccccgccgccccctacgacgcccgcagctctggctcctccggctgcccctcgcctacaccacagtcctctgaccccgacgacacgccccccaagctcctccccgagaccgtgagcccatccgcccccagctggcgcgagccggaggtgctcgacctgtccctccctcccgagtcggcagccaccagcaagcgggcaccgcctgaggtcacagctgctgccggcccggcacctcccacggcccctgagcccgccggtgcctcctccgagcccgaggctggggactggcgccccgagatgtcaccctgctccaatgtggtcgtcaccgatgtcaccagcaacctcctgacggtcacaatcaaggaattctgcaaccctgaggatttcgagaaggtggctgctggggtagcaggcgccgctgggggcggtggcagcattggggcga

SEQ ID NO: 68 - DNA, 1647 bp, Homo sapiens
gaattctgtgataagttattcaacttatgaaattcaagttacatgtgaattctgccaggcaatacaaggacctgtggaatatgagtgatgacaaaccctttctatgtactgcgcctggatgtggccagcgttttaccaacgaggatcatttggctgtccataaacataaacatgagatgacactgaaatttggtccagcacgtaatgacagtgtcattgtggctgatcagaccccaacaccaacaagattcttgaaaaactgtgaagaagtgggtttgtttaatgagttggcgagtccatttgagaatgaattcaagaaagcttcagaagatgacattaaaaaaatgcctctagatttatcccctcttgcaacacctatcataagaagcaaaattgaggagccttctgttgtagaaacaactcaccaggatagtcctttacctcacccagagtctactaccagtgatgagaaggaagtaccattggcacaaactgcacagcccacatcagctattgttcgtccagcatcattacaggttcccaatgtgctgcttacaagttctgactcaagtgtaattattcagcaggcagtaccttcaccaacctcaagtactgtaatcacccaggcaccatcctctaacaggccaattgtccctgtaccaggcccatttcctcttctgttacatcttcctagtggacaaaccatgcctgttgctattcctgcatcaattacaagttctaatgtgcatgttccagctgcagtcccactcgttcgaccagtcaccatggtgcctagtgttccaggaatcccaggtccttcctctccccaaccagtacagtcagaagcaaaaatgagattaaaagctgctttgacccagcaacatcctccagttaccaatggtgatactgtcaaaggtcatggtagcggattggttaggactcagtcagaggaatctcgaccgcagtcattacaacagccagccacatccactacagaaactccggcttctccagctcacacaactccacagacccaaagtacaagtggtcgtcggagaagagcagctaacgaagatcctgatgaaaaaaggagaaagtttttagagcgaaatagagcagcagcttcaagatgccgacaaaaaaggaaagtctgggttcagtctttagagaagaaagctgaagacttgagttcattaaatggtcagctgcagagtgaagtcaccctgctgagaaatgaagtggcacagctgaaacagcttcttctggctcataaagattgccctgtaaccgccatgcagaagaaatctggctatcatactgctgataaagatgatagttcagaagacatttcagtgccgagtagtccacatacggaagctatacagcatagttcggtcagcacatccaatggagtcagttcaacctccaaggcagaagctgtagccacttcagtcctcacccagatggcggaccagagtacagagcctgctctttcacagatcgttatggctccttcctcccagtcacagccctcaggaagttgattaaaaacctgcagtacaacagtttagatactcattagtgacttcaaagggaaatcaaggaaagaccagtttccatttatgcgaaatctgtggttgtaaattt

SEQ ID NO: 69 - DNA, 1542 bp, Homo sapiens
ctgacgtcagcgggagagtattatggtctgtcgtgcgctggctgctgcttttctgctcctggaagcggcctaggggggaagcggcgagtcaacatggagctttcagcggtgggggagcgggtgttcgcggccgaagccctcctgaagcggcgcatacggaaaggacgcatggaatacctcgtgaaatggaagggatggtcgcagaagtacagcacatgggaaccggaggaaaacatcctggatgctcgcttgctcgcagcctttgaggaaagggaaagagagatggagctctatggccccaaaaagcgtggacccaagcccaaaaccttcctcctcaaagcgcaggccaaggcaaaggccaaaacttacgagtttcgaagtgactcagccaggggcatccggatcccctaccctggccgctcgccccaggacctggcctccacttcccgggcccgggagggccttcgaaacatgggtttgtccccgccagcgagcagcaccagcaccagcagcacctgccgcgcagaggcccctcgggaccgggaccgagaccgggatagggaccgggagcgggatcgagaaagggagagggagcgagagagggagcgggaacgtgagagggaacgagagcggggtaccagcagagtggatgacaagcccagctcaccgggggacagctcgaagaagcgaggccccaagccccggaaggagctcccggacccctcacagaggcccttaggcgaacccagcgccggcctcggagagtacctcaagggcaggaagctggacgacaccccttccggggcaggaaagtttccagccggccacagtgtgatccagctggcccgaagacaggactcggacctggtgcagtgtggtgtgaccagccctagctcagctgaggccacgggcaaactggctgtggacaccttcccggccagggtgataaagcacagggctgccttcctggaggccaaaggccagggtgccctagatcccaatggcacccgggtccgacatggctcaggcccccccagctctggggggggcctgtaccgggacatgggggcccaggggggaaggccctccctcatcgccaggatccctgtggccagaatcctgggggacccggaggaagagtcctggagcccctccctgactaacctggagaaggtggtggtcacggacgtgacctcaaactttttgaccgtcaccattaaggaaagtaacacggaccaaggcttttttaaagagaaaagatgaatgctgggtgggtgtgtccaggaggagagcaggggagagagtgagcgtgagcttggcatagtgatttttatttctgggtgggatgtggccttttggctggtcccgtccctgatgtcacccccaccccaccagcccctttcatccctccttcctccccctcagtttttgttggaaagattatctctagagttatattttctattagatgtaaatatgttatttaagaaaaatatctaaatatatatatttcaactcgaaaaaaaaaaaaaaaaactcgag

SEQ ID NO: 70 - DNA, 2260 bp, Homo sapiens
ctcctcctgccgctgccgctgctttggctgctgcgtcatacgccccagagccgccgggacggaggggctgggcctggggaccccccggcctccgcctgcacgcccccccacgcccggacgtgccctctccgcgcgggggactcgcctaggtctcctacgtctgcccctgcccggctcccggcggccccagctgtcaccggcccccccaggatgcaatggcgcagcccccccggctgagccgctctggtgcctcctcactttgggacccagcttctcctgctcccacctctggccccaggcctcggctttgggagggtcaagatgtgctggccagatggactgatgggctgctatacttgggtaccatcaaaaaggtggacagtgctagggaggtgtgtctggtccagtttgaggatgattcgcagtttctggttctatggaaagacattagccctgctgccctccctggagaggaactcctctgttgtgtctgtcgctctgagactgtggtccctgggaaccggctggtcagctgtgagaagtgtcgccatgcttatcaccaggactgccatgttcccagggctccagcccctggagagggagagggcacatcctgggtatgccgccagtgtgtctttgcgatcgccaccaagaggggaggtgccctgaagaagggcccctatgcccgggccatgctgggtatgaagctttctctgccatatggactgaaggggctggactgggatgctggacatctgagcaaccgacagcagagttactgttactgtggtggccctggggagtggaacctgaaaatgctgcagtgccggagctgcctgcagtggttccatgaggcctgcacccagtgtctgagcaagcccctcctctatggggacaggttctatgaatttgaatgctgtgtgtgtcgcgggggccctgagaaagtccggagactacagcttcgctgggtggatgtggcccatcttgtcctgtatcacctcagtgtttgctgtaagaagaaatactttgattttgatcgtgagatcctccccttcacttctgagaattgggacagtttgctcctgggggagctttcagacacccccaaaggagaacgttcttccaagctcctctctgctcttaacagccacaaggaccgtttcatttcagggagagagattaagaagaggaaatgtttgtttggtctccatgctcggatgcctccccctgtggagccccctactggagatggagcactcaccagcttcccttcagggcagggccctgggggaggggtctcacgtcccctggggaagcgccggaggccggagccagagcccctgaggaggaggcagaaggggaaagtggaggagctggggccaccctcagcagtgcgcaatcagcccgagccccaggagcagagggagcgggctcatctgcagagggcactgcaggcctcagtgtctccaccatcccccagccctaaccagagttaccagggcagcagcggctacaacttccggcccacagatgcccgctgcctgcccagcagccccatccggatgtttgcttccttccacccttctgccagcaccgcagggacctctggggacagtggacccccagacaggtcacccctggaacttcacattggtttccccacagacatccctaaaagtgccccccactcgatgactgcctcatcttcctcagtttcatccccatccccaggtcttcctagacgctcagcacccccttctcccctgtgccgtagtttgtctcctgggactgggggaggagtccgaggtggggttggttacctgtcccgaggggaccctgtccgggtccttgctcggagagtacggcctgatggctctgtgcagtacctggttgagtggggaggagggggcatcttctgaacagcctgcctctgcccagctccccattcacacacaccggcactttcataccctgacctctgacctcacctacagctgggatgtacctggagagatagggggtagttctccctactgcccaggctggaatccaagagtggggagtggggaagaggccctcttctctaccctccttcatgattcctgacccctcccatccttcccatttcctttgatgttattttgttacagctttttaaatattttttaaaattatttaacccctgggggcagagactgaggagggaggatgataagggatcccggactctgtatgattgaaataaagagaaataaacaaa

SEQ ID NO: 71 - DNA, 3787 bp, Homo sapiens
gcggccgctcggcggccgggggtcccttcggtggggccgcggctccccgcccgccgcccccgcgcgtccattcgctttgtgtcccgcgcgcggccgggccccccgcgcactctcagccctgcgccccgcggcccggcgggcggctcccggcgcggccccagcagcccgcgccggcattgtgtggacgcgcccggccgcgagcgcgcgcgcgggccctgccgagcgcccccggccccgtccgctccggccgcggcgcccgcgcccgccgcccccgccgccctcgccgcgcggcccccggcccggcccggcccgacccgggcagcgcagcggcggggcgagcggcggcgcggcaacatggcgacggtgcccgtgtactgcgtctgccggctgccctacgacgttacccgctttatgatcgagtgcgatgcctgcaaggactggttccacggcagctgtgttggggtggaagaggaagaggcaccagacatcgacatttaccactgcccgaactgcgagaaaacccatggcaagtccacactcaagaaaaagcggacttggcacaaacacggccctgggccaacaccggacgtgaaaccagtgcagaatggcagtcagctgttcatcaaggagctgcggagccgaaccttccccagtgctgaagacgtggtgtcccgtgtgccaggtagccagctcaccgtgggctacatggaggagcatggcttcactgagcccatccttgtccccaagaaagatggcctgggcttagctgtccctgccccaacattctacgtgagtgacgtcgagaactacgtggggccggaacggagtgtggatgtgacagatgtcaccaagcagaaggactgcaagatgaagctgaaggagtttgtggactattactacagcaccaaccgcaagcgggtcctcaacgtcaccaacctcgagttctctgacacccgaatgtccagcttcgtggagccacctgacattgtaaagaaactgtcatgggtagaaaactactggccagatgatgcattgctggccaagcccaaagtgaccaagtactgcctaatctgcgtgaaggacagttacaccgacttccacatcgactctgggggcgcctctgcctggtaccacgtgctcaagggggagaagaccttctatctcatcaggccggcctcggccaacatctccctgtatgagcgctggcggtctgcctctaaccacagcgagatgttctttgctgaccaggtcgacaaatgctacaagtgcatcgtcaagcagggccagaccctcttcatcccctcaggctggatctacgccacactcacccctgtggactgcctggccttcgcgggacatttcctccacagcctgagtgtggagatgcagatgagagcatacgaggtggaaaggaggttgaaacttggcagcctgactcagtttcccaactttgaaactgcgtgctggtacatgggaaagcacctattggaggcgttcaaaggttctcacaagtctgggaagcagctgcccccacatctagtccaaggagctaaaattctcaatggtgctttccgatcgtggacgaagaagcaggctttggcagagcatgaggacgagctcccggagcacttcaaaccttcacagctaatcaaggacctggccaaagagatccggctcagtgagaatgcctccaaagccgtccgaccggaagtgaatactgtcgcctcgtcagatgaggtgtgtgacggggaccgggagaaggaggagcccccgtctcccattgaggccaccccgcctcaatccctcctggagaaagtgtccaaaaaaaagactcccaaaactgtgaagatgcccaagccatccaaaatccccaagcccccgaagccccctaagcccccaaggccccccaaaacgctgaagctcaaagatggaggcaagaagaaagggaagaagtcccgggagtcagcctcacccaccatccccaacctggacctgctcgaagcccacaccaaggaggcactgaccaagatggagccgcccaagaagggcaaggccacaaagagtgtcctgagtgtgcccaacaaagatgtggttcacatgcagaatgatgtggagaggctggaaattcgagagcaaacaaagagcaagtcagaagccaagtggaaatacaagaacagcaaacctgactcgttactgaagatggaggaggagcagaggctggagaagtcgcccctggctgggaacaaggacaagttttccttttctttctccaacagaaaactcctgggctccaaggccctcaggcccccgagcagccctggtgtgttcggcgccttgcagagcttcaaggaggacaaggccaagcccgtgcgcgatgagtatgagtacgtatcagatgatggggagctgaagatagacgagtttcccatcaggaggaagaagagcgcccccaaaagggacttgtccttcttgttagacaagaaggaggctctcctcatgcccacctcgaagccaaagctggattctgcggtgtacaagagcgatgactcctctgacgagggctctctgcacatcgacacggacaccaagccaggcagaaatgccaaagtgaagaaggagagtgggagctccgcggccggcatcctggacctgctgcaggccagcgaggaggttggcgcactcgagtacaaccccaacagccagccccctgcctcccccagcacacaggaagccattcagggaatgctctccatggccaatctgcaggcctctgactcttgcctgcagaccacatggggcacggggcaggccaagggtggctcactggcagcccatggtgcccggaagattggtggtggcaacaaaggcacaggcaagcgcctgctgaagaggactgccaagaacagtgtggatctggaggactacgaggagcaggatcacctggatgcctgcttcaaggactcagactatgtttacccctcactggagtctgacgaagataaccccgtcttcaagtcccggtcaaagaagaggaaaggctcagacgatgctccgtacagccccacagccagggtcggtccatcggtgccaagacaagacaggcctgtgcgtgaggggaccagagtggcctccattgagacggggctggcagctgctgcagccaagctgtcccagcaggaggagcagaaaaacaggaagaagaagaacaccaaaaggaagccggctcctaacactgcctccccctccatctccacctctgcctccgcctccacgggtaccacctcggcctccaccaccccagcatccaccaccccggcctccaccaccccagcatccaccaccccggcctccaccagcacagccagcagccaggcctcacaggagggcagctcacctgagcccccacctgaatcacacagcagtagcctggctgaccacgaatatacagcagccggcacattctcggggtcccaggctggccgtgcctcccagcccatggcccctggagtctttctcacacagaggcggccttctgcatcatcccccaacaacactgctgccaaaggaaaacgtacaaaaaagggcatggccaccgccaagcaaaggcttggaaagatcttgaagatccatcggaatgggaaactgctcctctaaggcttggaaagccaggatccttctgatatgctaaggacccccggagccccgctacatcagcccctcccaggacggtggctgtgccgcctggcccggggagggcttgcttcattccgaccaattttccaatcaa

SEQ ID NO: 72 - DNA, 4058 bp, Homo sapiens
cccgccctcggcgcccccgcccctccagaaaggggaggaggcgaggggagcccgccgcggaggccgagcgagccggcgcccagcccagcctggcgactggggaccccggcacatgaggtggacgccccggggaagacttgggtgcacagccaggcgagaaggtcttgagtcagacagagcaccagccttggggaccctggaccactatcatggagactgagagcgagcagaactccaattccaccaatgggagttctagctcagggggcagctctcggccccagatagctcaaatgtcactttatgaacgacaagcagtgcaggctctgcaagcactgcagcggcagcccaatgcagctcagtatttccaccagttcatgctccagcagcagctcagtaatgcccagctgcatagcctggctgccgtccagcaggccacaattgctgccagtcggcaggccagctccccaaacaccagcactacacagcagcagactaccaccacccaggcctcgatcaatctggccaccacatcggccgcccagctcatcagccgatcccagagtgtgagctctcccagtgctaccaccttgacccaatctgtgctactggggaacaccacctccccacccctcaaccagtctcaggcccagatgtatctacggccacagctgggaaacctattgcaggtaaaccgaacccctgggtcgaatgtgcctctagcctcccaactcatcctgatgcctaatggggcggtggctgcagtccagcaggaggtgccatctgctcagtctcctggagttcatgcagatgcagatcaggttcagaacttggcagtaaggaatcaacaggcctcagctcaaggacctcagatgcaaggctccactcagaaggccattcctccaggagcctcccctgtctctagcctctcccaggcctctagccaggccctagcggtggcacaggcttcctctggggccacaaaccagtccctcaaccttagtcaagctggtggaggcagtgggaatagcatcccagggtccatgggtccaggtggaggtgggcaggcacatggtggtttgggtcagttgccttcctcaggaatgggtggtgggagctgtcccaggaagggtacaggagtggtgcagcccttgcctgcagcccaaacagtaactgtgagccagggcagccagacagaggcagaaagtgcagcagccaagaaggcagaagcagatgggagtggccagcagaatgtgggcatgaacctgacacggacagccacacctgcgcccagccagacacttattagctcagccacctacacacagatccagccccattcactgattcagcaacagcaacagatccacctccagcagaaacaggtggtgatccagcagcagattgccatccaccaccagcagcagttccagcaccggcagtcccagctccttcacacagctacacacctccagttggcgcagcagcagcagcagcaacaacagcaacagcagcaacagcagcagccgcaagccaccaccctcactgcccctcagccaccacaggtcccacctactcagcaggtcccaccttcccagtcccagcagcaagcccaaaccctggtcgttcagcccatgcttcagtcttcacccttgtctcttccacctgatgcagcccctaagccaccaattcccatccaatccaaaccacctgtagcacctatcaagccgcctcagttaggggccgctaagatgtcagctgcccagcaaccaccaccccatatccctgtgcaagttgtaggcactcgacagccaggtacagcccaggcacaggctttggggttggcacagctggcagctgctgtacctacttcccgggggatgccaggtacagtgcagtctggtcaggccctaaaggcttcctcgccaccttcatcccaggctcctggtgcactgcaggagtgccctcccacattggccccggggatgacccttgctcctgtgcaggggacagcacatgtggtaaagggtggggctaccacctccacacctgttgtagcccaggtccctgctgccttctatatgcagtctgtgcacttgccgggaaaaccccagacattggctgtcaaacgcaaggctgactctgaggaggagagagatgatgtctccacattgggttcaatgcttcctgccaaggcatctccagtagcagaaagcccaaaagtcatggacgagaagagcagtcttggagaaaaagctgaatcagtggctaatgtgaatgctaatactccaagcagtgaactagtagccttgacccccgccccttcagtaccgcctcctacactagccatggtgtctagacaaatgggtgactcaaaacccccacaggccatcgtgaagccccagattctcacccacatcattgaaggctttgttatccaggaaggagcagaacctttccccggtggttgttctcagttactgaaggagtctgagaagccactacagactggccttccgacaggcttgactgagaatcagtcaggtggcccctttggagtggacagcccatctgctgagttagataagaaggcgaatctcctgaagtgcgagtactgtgggaagtacgcccccgcagagcagtttcgtggctctaagaggttctgctccatgacctgcgctaagaggtacaatgtgagctgtagccatcagttccggctgaagaggaaaaaaatgaaagagtttcaagaagccaactatgctcgcgttcgcaggcgtggaccccgccgcagctcctctgacattgcccgtgccaagattcagggcaagtgccaccggggtcaagaagactctagccggggttcagataattccagttatgatgaagcactctctccaacatctcctgggcctttatcagtaagagctgggcatggagaacgtgacctggggaatcccaatacagctccacctacaccggaattacatggcatcaaccctgtgttcctgtccagtaatcccagccgttggagtgtagaggaggtgtacgagtttattgcttctctccaaggctgccaagagattgcagaggaatttcgctcacaggagattgatggacaggcctttttattacttaaagaagaacatcttatgagtgccatgaacatcaagctgggccctgccctcaagatctgtgccaagatcaatgtcctcaaggagacctaaggtggccctcttgcacaaaccagcctaaggcagacactctccactgtccaggttataacctggtaccagcagactttgcagggaagaaagagttgttccaatcatgtaaccttctgtaggggattactgagacagggaagagaagtgcaagaattggttgctggtgctacatggcggcagctttgacattttctctgggttctactttattttttaaaatctttacagttctcaccatttcacgtaccttaatccaatctttataaaagaggcagtctagagaactaggactgctcagccttatcctggagtggagcatttagcccaggtcttaattctccaagaggaggaatacatagtatggtaaggcaaggaactgggtggaatgtcaggttgcctgcccaatgggagaggtagggtttttctagcttgtgtgacagaagtagcaaaatctggtcctcccccctcccagtgtagctgtggctcagagttttttctttttgttgtcacttactcccttgtgattgaattttttctcctgcatccatggcaggatccccagccagtatagagacttggttggcatcttctgctgcagggactaaaagtatttgactggggcacatgtggctgttgtcattctttctgcatcccactgttcccctccaatttatgttattttctaccctgtttttcagttccatctctgctctgtcctatagctttataaaaccagagtgtgtggggctgaggtcaggagtataagtacctgccttaggcactattccttatataacaaaaatattaaatatttttttcctcagtaaaaggatg

SEQ ID NO: 73 - DNA, 2555 bp, Homo sapiens
ggcgccgcatgtgtctccgcggcggctgcagccctcgagcgcccgccgccgcgccccaaccccggccgccgcccgccctcccgccccggcctcgcgcccccgtcccggcctcgcgccccggccgccctttgttgacgccggccaggccgtgcggtcggatgcgccgcggcagccccgggccccggctcggaggctcccggggcgagaggaggcggcccgccggccgggaccccgcgcgagtcggccccggccaggggctgcgtaggcccgcccggccaggcccagccgcctggacagagacagggcagggcattgttcatgcactgaccgacctcagcatccccggcatgacctcagggaacggaaactctgcctccagcatcgccggcactgccccccagaatggtgagaataaaccaccacaggccattgtgaaaccccaaatcctgacgcatgttatcgaagggtttgtgatccaggagggggcggacgtttcccggtgggacgctcgtctgctggtggggaatctcaagaagaagtatgcacaggggttcctgcctgagaaacttccacagcaggatcacaccaccaccactgactcggagatggaggagccctatctgcaagaatccaaagaggagggtgctcccctcaaactcaagtgtgagctctgtggccgggtggactttgcctataagttcaagcgttccaagcgcttctgttccatggcttgtgcaaagaggtacaacgtgggatgcaccaaacgggtgggacttttccactcagaccggagcaagctgcagaaggcaggagctgcgacccacaaccgccgtcggccagcaaagccagtctgccaccacttaccaaggataccaagaagcagccaacaggcactgtgcccctttcggttactgctgctttgcgtaacacacagccaggaagactccagccgttgctcagataactcaagctatgaggaacccttgtcacccatctcagccagctcatctacttccgccggcgacaaggccagcgggacctggagctccccgacatgcatatgcgggacctggtgggcatgggacaccacttcctgccaagtgagccaccaagtgaatgtagaagacgtctacgaattcatccgctctctgccaggctgccaggagatagcagaggaattccgtgcccaggaaatcgacgggcaagccctgctgctgctcaaggaggaccacctgatgagcgttatgaacatcaagctggggcccgccctgaagatctacgcccgcatcagcatgctcaaggactcctagggctggtggcaccaggattctggcccagggcgcctcctcccgactgagcagagccagacagacattcctgaggggcccagaaatggcggcgttggagggcaggggctctccctaggggcatagctggtgaggaggtctgggcacctcctccatggctctcaggggcctttcatttctgtgggaggggcagagaggtaggtggcacagaagatggggctttatgcttgtaaatattgatagcactggcttcctccaaagtcccaatactctagccccgctctcttcccctctttctgtcccccattttccagggggtatatggtcagggctccccaacctgagttggttacttcaagggcagccagcaggcctggatggaggcctagaaagcccttgccttccttcctcccacttctttctccaggcctggttaactcttccgttgtcagcttctcccccttcagcctgtttctgcagcagccagggttctcccccctacaccctctgcaggtggagagagagaagctgggcccagccgcggtgcctgctggccaagacgccttaacgctgtgtgtatgactgtgtgactgtgtgggagcctggactgacagataggccaagggctactctctggcatctccaggtgttttgtagcaaacagccacttagtgctttgtcctggactccactcagcctcaggatggggaatagccaagaatggcagcctcagcgcagaggcaaggtcagaaagagacggcgcttcagagtttcctttccagacacccctccccgcactgtgaagttcccctgaccgccctcctggttcacaaagagcattaagaaagctgcggtggtctgagcaacatagcccagacgtggagcctcctggcctgcctgcccgcccaccctgggagtccagtggtgaggctcagagaacttctaaggggaaagaacagctggagtttctgttgatgtgaagaaggcagctcttggcctcccactcccacacttctttgcctataaatcttcctagcagcaatttgagctacctgaggaggaggcagggcagaagggcaagggcctgcctctgacctgccgtgtcctttgcaggaaggaggtaggcacctttctgagcttattctattccccacccacacccccaggcagggttggaaatgaaggacttttttaacctttgttttgttttttaaaaataaatctgtaaaatctgaaaaaaaaaaaaaa

SEQ ID NO: 74 - DNA, 766 bp, Homo sapiens
cactttgtcaccaactgctgccaactcgccaccactgctgccgcaatcgcaaccactgctttgtctctgaagtgagactgctcctggtgccatgaacggagacgacacctttgcaaagagacccagggatgatgctaaagcatcagagaagagaagcaaggcctttgatgatattgccacatacttctctaagaaagagtggaaaaagatgaaatactcggagaaaatcagctatgtgtatatgaagagaaactataaggccatgactaaactaggtttcaaagtcaccctcccacctttcatgtgtaataaacaggccacagacttccaggggaatgattttgataatgaccataaccgcaggattcaggttgaacatcctcagatgactttcggcaggctccacagaatcatcccgaagatcatgcccaagaagccagcagaggacgaaaatgattcgaagggagtgtcagaagcatctggcccacaaaacgatgggaaacaactgcaccccccaggaaaagcaaatatttctgagaagattaataagagatctggacccaaaagggggaaacatgcctggacccacagactgcgtgagagaaagcagctggtgatttatgaagagatcagtgaccctgaggaagatgacgagtaactcccctgggggatacgacacatgcccttgatgagaagcagaacgtggtgacctttcacgaacatgggcatggctgcggctccctcgtcatcaggtgcatagcaagtg

SEQ ID NO: 75 - DNA, 766 bp, Homo sapiens
ctctctttcgattcttccatactcagagtacgcacggtctgattttctctttggattcttccaaaatcagagtcagactgctcccggtgccatgaacggagacgacgcctttgcaaggagacccacggttggtgctcaaataccagagaagatccaaaaggccttcgatgatattgccaaatacttctctaaggaagagtgggaaaagatgaaagcctcggagaaaatcttctatgtgtatatgaagagaaagtatgaggctatgactaaactaggtttcaaggccaccctcccacctttcatgtgtaataaacgggccgaagacttccaggggaatgatttggataatgaccctaaccgtgggaatcaggttgaacgtcctcagatgactttcggcaggctccagggaatctccccgaagatcatgcccaagaagccagcagaggaaggaaatgattcggaggaagtgccagaagcatctggcccacaaaatgatgggaaagagctgtgccccccgggaaaaccaactacctctgagaagattcacgagagatctggacccaaaaggggggaacatgcctggacccacagactgcgtgagagaaaacagctggtgatttatgaagagatcagcgaccctgaggaagatgacgagtaactcccctcagggatacgacacatgcccatgatgagaagcagaacgtggtgacctttcacgaacatgggcatggctgcggacccctcgtcatcaggtgcatagcaagtg

SEQ ID NO: 76 - DNA, 2511 bp, Homo sapiens
atgagtaaactgtcgtttcgggcgcgggcgctagacgcctcgaagccgctgccggttttccgctgtgaggatctgcccgacctgcacgaatacgcctcgataaacagggccgtgccgcagatgcccaccggaatggagaaggaagaggagtcggaacatcatcttcagcgggctatttcagcacagcaggtgtatggcgagaagagggataatatggttataccggtcccagaggcagaaagtaatattgcttactatgagtctatatatcctggggaatttaagatgccaaagcagctcattcacatacagccttttagtttggatgctgaacagcctgattatgatttggattctgaagatgaagtatttgtgaataaactgaaaaagaaaatggacatctgcccattgcaatttgaggagatgattgaccgcctagaaaaaggcagtggtcagcagccagtcagtctgcaggaagccaaactactgctaaaagaagatgatgaactaattagagaagtttatgaatattggattaaaaagagaaaaaactgtcgagggccatctcttattccatcagtaaaacaagagaagcgagatggttccagcacaaatgatccttatgtggcttttagaaggcgtactgaaaaaatgcagactcgaaaaaatcgcaaaaatgatgaagcctcttacgaaaaaatgcttaagctgcgacgagatctaagtcgagctgttactattctagagatgataaaaagaagagaaaaaagtaaaagagagctattgcacttaacactggaaattatggaaaagaggtataatttgggcgactacaatggagagatcatgtctgaggttatggcacagagacagccaatgaaacctacttatgccatccccatcatccctattactaatagcagtcaatttaaacaccaggaagcaatggatgtgaaggagttcaaagttaataagcaagataaagccgatcttatccgaccgaaacggaaatatgaaaagaagcccaaagtcttaccatcgtctgccgctgctactccccaacagacgagtcctgctgcactgccagtcttcaatgctaaagatctgaatcagtatgactttcccagctcagacgaagaacctctctcccaggttttgtctggctcttcggaagctgaggaagacaatgatcctgatggtccttttgctttccgtaggaaagcaggctgtcagtactatgctcctcacttagaccaaactggcaactggccttggactagtcctaaagatggaggattaggggatgtgcgatatagatactgcttaactactctcaccgtaccccaaaggtgtattggatttgcacgaagacgggttgggcgcggtggaagggtcttactggacagagctcattcagactatgacagtgtgtttcaccatctggatttggaaatgctttcctcaccacaacattctccagtcaatcagtttgccaatacctcagaaacaaatacctcggacaaatctttctctaaagacctcagtcagatactagtcaatatcaaatcatgtagatggcggcattttaggcctcggacaccatccctacatgacagtgacaatgatgaactctcctgtagaaaattatataggagtataaaccgaacaggaacagcacaacctgggacccagacatgcagtacctctacgcaaagtaaaagtagcagtggttcagcacactttgcatttacagccgaacaataccagcaacatcaacagcaactggcactcatgcagaaacagcagcttgcacaaattcagcaacagcaagcaaatagtaattcctccaccaacacatcacagaaccttgcatctaaccagcagaaaagtggctttcgcctgaatatacagggtttagaaagaacactacagggttttgtttctaagactttggattctgctagtgcacagtttgctgcttctgctttggtgacatcagaacaactgatgggattcaagatgaaggatgatgtggtgcttggaatcggggtgaatggcgtccttccagcctcaggagtatacaagggcttacacctcagtagtactacaccaacagcacttgtacatacaagtccatcaacggcaggttcagctttgttacagccttcaaatattacacagacttcaagttcccacagtgcactgagtcatcaagtaactgctgccaattctgcaacaactcaggttctgattgggaacaacattcgattaactgtaccttcatcagttgccactgtaaactctattgccccaataaatgcacgacatatacctaggactttaagtgctgttccatcatctgccttaaagctggccgctgcagcaaactgtcaagtttccaaggtcccatcttcatcctctgtagattcagttccaagggaaaatcatgaatcagaaaagccagcactgaacaacatagcagacaacacagtagcgatggaggtgacgtag

SEQ ID NO: 77 - DNA, 2594 bp, Homo sapiens
gaggctggacacctgttctgctgttgtgtcctgccattctcctgaagaacagaggcacactgtaaaacccaacacttccccttgcattctataagattacagcaagatggaaataccaaatccccctacctccaaatgtatcacttactggaaaagaaaagtgaaatctgaatacatgcgacttcgacaacttaaacggcttcaggcaaatatgggtgcaaaggctttgtatgtggcaaattttgcaaaggttcaagaaaaaacccagatcctcaatgaagaatggaagaagcttcgtgtccaacctgttcagtcaatgaagcctgtgagtggacacccttttctcaaaaagtgtaccatagagagcattttcccgggatttgcaagccaacatatgttaatgaggtcactgaacacagttgcattggttcccatcatgtattcctggtcccctctccaacagaactttatggtagaagatgagacggttttgtgcaatattccctacatgggagatgaagtgaaagaagaagatgagacttttattgaggagctgatcaataactatgatgggaaagtccatggtgaagaagagatgatccctggatccgttctgattagtgatgctgtttttctggagttggtcgatgccctgaatcagtactcagatgaggaggaggaagggcacaatgacacctcagatggaaagcaggatgacagcaaagaagatctgccagtaacaagaaagagaaagcgacatgctattgaaggcaacaaaaagagttccaagaaacagttcccaaatgacatgatcttcagtgcaattgcctcaatgttccctgagaatggtgtcccagatgacatgaaggagaggtatcgagaactaacagagatgtcagaccccaatgcacttccccctcagtgcacacccaacatcgatggccccaatgccaagtctgtgcagcgggagcaatctctgcactccttccacacacttttttgccggcgctgctttaaatacgactgcttccttcacccttttcatgccacccctaatgtatataaacgcaagaataaagaaatcaagattgaaccagaaccatgtggcacagactgcttccttttgctggaaggagcaaaggagtatgccatgctccacaacccccgctccaagtgctctggtcgtcgccggagaaggcaccacatagtcagtgcttcctgctccaatgcctcagcctctgctgtggctgagactaaagaaggagacagtgacagggacacaggcaatgactgggcctccagttcttcagaggctaactctcgctgtcagactcccacaaaacagaaggctagtccagccccacctcaactctgcgtagtggaagcaccctcggagcctgtggaatggactggggctgaagaatctctttttcgagtcttccatggcacctacttcaacaacttctgttcaatagccaggcttctggggaccaagacgtgcaagcaggtctttcagtttgcagtcaaagaatcacttatcctgaagctgccaacagatgagctcatgtacccctcacagaagaagaaaagaaagcacagattgtgggctgcacactgcaggaagattcagctgaagaaagataactcttccacacaagtgtacaactaccaaccctgcgaccacccagaccgcccctgtgacagcacctgcccctgcatcatgactcagaatttctgtgagaagttctgccagtgcaacccagactgtcagaatcgtttccctggctgtcgctgtaagacccagtgcaataccaagcaatgtccttgctatctggcagtgcgagaatgtgaccctgacctgtgtctcacctgtggggcctcagagcactgggactgcaaggtggtttcctgtaaaaactgcagcatccagcgtggacttaagaagcacctgctgctggccccctctgatgtggccggatggggcaccttcataaaggagtctgtgcagaagaacgaattcatttctgaatactgtggtgagctcatctctcaggatgaggctgatcgacgcggaaaggtctatgacaaatacatgtccagcttcctcttcaacctcaataatgattttgtagtggatgctactcggaaaggaaacaaaattcgatttgcaaatcattcagtgaatcccaactgttatgccaaagtggtcatggtgaatggagaccatcggattgggatctttgccaagagggcaattcaagctggcgaagagctcttctttgattacaggtacagccaagctgatgctctcaagtacgtggggatcgagagggagaccgacgtcctttagccctcccaggccccaacggcagcacttatggtagcggcactgtcttggctttcgtgctcacaccactgctgctcgagtctcctgcactgtgtctcccacactgagaaaccccccaacccactccccctgtagtgaggcctctgccatgtccagagggcacaaaactgtctcaatgagaggggagacagaggcagctagggcttggtctcccaggacagagagttacagaaatgggagactgttt

SEQ ID NO: 78 - DNA, 2576 bp, Homo sapiens
gaattccgggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaattttggaggccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgtttgttgattacagatacagccaggctgatgccctgaagtatgtcggcatcgaaagagaaatggaaatcccttgacatctgctacctcctccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgaaaaaaaaaaaaaaaaaa

SEQ ID NO: 79 - DNA, 3203 bp, Homo sapiens
gagaggcagagatcggggcgagacaatggggatgtgggcgcgggagccccgttccggcttagcagcacctcccagccccgcagaataaaaccgatcgcgccccctccgcgcgcgccctcccccgagtgcggagcgggaggaggcggcggcggccgaggaggaggaggaggaggccccggaggaggaggcgttggaggtcgaggcggaggcggaggaggaggaggccgaggcgccggaggaggccgaggcgccggagcaggaggaggccggccggaggcggcatgagacgagcgtggcggccgcggctgctcggggccgcgctggttgcccattgacagcggcgtctgcagctcgcttcaagatggccgcttggctcgcattcattttctgctgaacgacttttaactttcattgtcttttccgcccgcttcgatcgcctcgcgccggctgctctttccgggattttttatcaagcagaaatgcatcgaacaacgagaatcaagatcactgagctaaatccccacctgatgtgtgtgctttgtggagggtacttcattgatgccacaaccataatagaatgtctacattccttctgtaaaacttgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccaatggctctaatgaagatagaggagaggttgcagatgaagataagagaattataactgatgatgagataataagcttatccattgaattctttgaccagaacagattggatcggaaagtaaacaaagacaaagagaaatctaaggaggaggtgaatgataaaagatacttacgatgcccagcagcaatgactgtgatgcacttaagaaagtttctcagaagtaaaatggacatacctaatactttccagattgatgtcatgtatgaggaggaacctttaaaggattattatacactaatggatattgcctacatttatacctggagaaggaatggtccacttccattgaaatacagagttcgacctacttgtaaaagaatgaagatcagtcaccagagagatggactgacaaatgctggagaactggaaagtgactctgggagtgacaaggccaacagcccagcaggaggagttccctccacctcttcttgtttgcctagccccagtactccagtgcagtctcctcatccacagtttcctcacatttccagtactatgaatggaaccagcaacagccccagcggtaaccaccaatcttcttttgccaatagacctcgaaaatcatcagtaaatgggtcatcagcaacttcttctggttgatacctgagactgttaaggaaaaaaattttaaacccctgatttatatagatatcttcagccattacgactttctagagctaatacatgtgactatcgtccaatttgctttcttttgtagtgacattaaatttggctataaaagatggactacatgtgatactcctgtccgtcttggttcaaaagaaagattgttgttataaagaattggtttcttggaaagcaggcaagactttttctctgtgttaggaaagatgggaaatggtttctgtaaccattgtttggatttggaagtactctgcagtggacataagcattgggccatagtttgttaatctcaactaacgcctacattacattctccttgatcgttcttgttattacgctgttttgtgaacctgtagaaaaacaagtgctttttatcttgaaattcaaccaacggaaagaatatgcatagaataatgcattctatgatgccatgtcactgtgaataacgatttcttgcagctatttagccattttgattgctgtttgatttatacttctctgttgctacgcaaaaccgatcaaagaaaagtgaacttcagttttacaatctgtatgcctaaaagcgggtactaccgtttattttactgacttgtttaaatgattcgcttttgtaagaatcagatggcattatgcttgttgtacaatgccatattggtatatgacataacaggaaacagtattgtatgatatatctataaatgctataaagaaatattgtgtttcatgcattcagaaatgattgttaaaattctcccaactggttcgacctttgcagatacccataacctatgttgagccttgcttaccagcaaagaatatttttaatgtggatatctaattctaaagtctgttccattagaagcaattggcacatctttctatactttatatacttttctccagtaatacatgtttactttaaaaattgttgcagtgaagaaaaacctttaactgagaaatatggaaaccgtcttaattttccattggctatgatggaattaatattgtattttaaaaatgcatattgatcactataattctaaaacaattttttaaataaaccagcaggttgctaaaagaaggcattttatctaaagttattttaataggtggtatagcagtaattttaaatttaagagttgcttttacagttaacaatggaatatgccttctctgctatgtctgaaaatagaagctatttattatgagcttctacaggtatttttaaatagagcaagcatgttgaatttaaaatatgaataaccccacccaacaattttcagtttattttttgctttggtcgaacttggtgtgtgttcatcagttatttgtgagggtgtttattctatatgaatattgtttcatgtttgtagggaaattgtagctaaacatttcattgtccccagtctgcaaaagaagcacaattctattgctttgtcttgcttatagtcattaaatcattacttttacatatattgctgttacttctgctttctttaaaaatatagtaaaggatgttttatgaagtcacaagatacatatatttttattttgacctaaatttgtacagtcccattgtaagtgttgtttctaattatagatgtaaaatgaaatttcatttgtaattggaaaaaatccaataaaaaggatattcatttagaaaatagctaagatctttaataaaaatttgatatgaaa

SEQ ID NO: 80 - DNA, 2227 bp, Homo sapiens
gagagcccgaacaggaagagggtacagctttgtgcaggtcacatgcccactgcagccctccagcctctggtccccagagcggactttggaagctgaactgcttttgttgctggaagacttatgttataatttaccctgggtggaccagggtcgtacaaaagggcaacgctccccagtccccccactcccgaccccggaatcatgcatcggactacacggatcaaaatcacagagctgaacccccacctcatgtgtgccctctgcggggggtacttcatcgacgccaccactatcgtggagtgcctgcattccttctgcaaaacctgcatcgtgcgctacctggagaccaacaaatactgccccatgtgtgacgtgcaggtccataaaacccggccgctgctgagcatcaggtctgacaaaacacttcaagacattgtctacaaattggtccctgggctttttaaagatgagatgaaacggcggcgggatttctatgcagcgtaccccctgacggaggtccccaacggctccaatgaggaccgcggcgaggtcttggagcaggagaagggggctctgagtgatgatgagattgtcagcctctccatcgaattctacgaaggtgccagggaccgggatgagaagaagggccccctggagaatggggatggggacaaagagaaaacaggggtgcgcttcctgcgatgcccagcagccatgaccgtcatgcatcttgccaagtttctccgcaacaagatggatgtgcccagcaagtacaaggtggaggttctgtacgaggacgagccactgaaggaatactacaccctcatggacatcgcctacatctacccctggcggcggaacgggcctctccccctcaagtaccgtgtccagccagcctgcaagcggctcaccctagccacggtgcccaccccctccgagggcaccaacaccagcggggcgtccgagtgtgagtcagtcagcgacaaggctcccagccctgccaccctgccagccacctcctcctccctgcccagcccagccaccccatcccatggctctcccagttcccatgggcctccagccacccaccctacctcccccactcccccttcgacagccagtggggccaccacagctgccaacgggggtagcttgaactgcctgcagacaccatcctccaccagcagggggcgcaagatgactgtcaacggcgctcccgtgccccccttaacttgaggccagggaccctctcccttcttccagccaagcctctccactccttccactttttctgggcccttttttccacttcttctactttccccagctcttcccaccttgggggtggggggcgggttttataaataaatatatatatatatgtacataggaaaaaccaaatatacatacttattttctatggaccaaccagattaatttaaatgccacaggaaacaaactttatgtgtgtgtgtatgtgtggaaaatggtgttcattttttttggggggggtcttgtgtaatttgctgtttttgggggtgcctggagatgaactggatgggccactggagtctcaataaagctctgcaccatcctcgctgtttcccaaggcaggtggtgtgttgggggccccttcagacccaaagctttaggcatgattccaactggctgcatataggagtcagttagaattgtttctttctctccccgtttctctccccatcttggctgctgtcctgcctctgaccagtggccgccccccgcgttgttgaatgtccagaaattgctaagaacagtgccttttacaaatgcagtttatccctggttctgaggagcaagtgcagggtggaggtggcacctgcatcacctcctcctcttgcagtggaaactttgtgcaaagaatagatagttctgcctcttttttttttttttcctgtgtgtgtggcctttgcatcatttatcttgtggaaaagaagattcaggccctgagaggtctcagctcttggaggagggctaaggctttagcattgtgaagcgctgcacccccaccaaccttaccctcaccggggaaccctcactagcaggactggtggtggagtctcacctggggcctagagtggaagtgggggtgggttaacctcacacaagcacagatcccagactttgccagaggcaaacagggaattccgccgatactgacgggctccaggagtcgtcgccacactcg

SEQ ID NO: 81 - DNA, 2635 bp, Homo sapiens
atcactggtctcgcgtgcgcgtgaccaggcccggtttccggtgccaggacctttccgaagcgtcgagtggcctaacggtcacagctgtcgcccatcggagaggcaggactactgcgagcagttttaccgcgacctccggaggccggcgtgacaggctctgtcactaaaataggaaccgaatattgtatctgacgcatcctgtaatactgaagagcaactgaagacagttgatgatgtccttattcattgccaggttatatatgatgctctgcaaaacctggataagaagattgatgtgattcgtagaaaggtttcaaaaatccaacgtttccatgcgagatccctgtggacaaatcataagcgatatggatataaaaagcattcttaccggcttgttaaaaagcttaaactccagaaaatgaagaaaaatgaggtttacgagacattctcctaccctgaaagttacagccccactttaccagtgtcaaggcgtgagaataattccccgagcaaccttccaaggccatccttttgcatggaagaataccagcgagctgagctggaggaggacccgatcctcagccgcactccgagtccagtgcatccctcagatttctctgagcataattgtcagccgtattatgcatctgatggtgcaacgtatggttcttcttcagggctctgccttggcaaccctcgggctgacagcatccacaacacttactcaactgaccatgcttctgcagcaccaccttcagttacaaggtcaccagttgaaaatgacggttacatagaggaaggaagcatcactaagcacccttcaacctggtcggtggaagcagtggtcctatttctaaaacaaacagatcctcttgcattatgccctcttgtcgacctcttcagaagccatgaaattgacgggaaggctctgctcctactcacgagtgacgtgttgctgaagcacttgggggtgaagctgggaacggctgtgaagctatgctactacattgaccgacttaaacaaggaaaatgctttgaaaattgaaaaaatccttgtgcaaatttagattgggccaacttctagaggcaccaatgccttcttagtgtggaatcatttttctgccctttagtcgtttttgttttgtagaaagtatctctcaaaatatattatagctagaattgtagaactatgttatagtccagtctacttctttaaaaaccatttaaactgctagatagtattagaatagtccaatagaaaattcattctttataggtctttaaaaattacttttattatattgtttacaaatatatttcatgcaagaaacagaaaaaaaaaaaaaccctttgattctggttcatctcgatacagagaaccaaaacagctaagagaggtattatcagggttgacaactcctatgattgaatctatgggaattattcctcagaagagaatttaaaggtgtacccatatatatctctttctggagtattttatctgtctgatgttgcagtattctacaagtttccagaaagagaatagccatataaattattttcctttctgctattatttctctatatgttttatttattcagatttagagtaaaaaataagcatataaacttttattatgtgctcttaacagttttaagataaactataggatagatagaatggttattttatgcaagaaatattgtaccgcaagggtggtttggatgaagtctgactactttttttcaaacaaactattatattaaaactgtcatattttggctaagtttggacctataactacactttcattgtttgcatctctctatgaagatacgtctgtccaaacttttaaaaggcataactgtattttatgtgtttattctttatatagatagtattttatattttattctcacccgaagtattcacacaatctttttaaaaaaaatttgaaatggcattttgtattgccacagaggtaggatgagccatatattagtgaaatgttttattttgtaaaatataaatggattatttgccatcattagtacctctcaacttactttttagaggacaagaaacaatctgtagattggtttccatacagggaagttctccgtcctatgcaatgtttctaattaatttgcttaattctgagccattaatcctgctacactttgaatgatacattaattcagactaatctttgggggctttattttgtaagttagaactttcaagggaaacatgttcaacactattattttgttataaatttataactttgttattacattgtgtaacaaatataaggtttacgagctatgagaattggtgctatcaccattagctatttgctgtaatgtcaagaaaatgttcaccagatgcaagaatgtaccttttctttttagaaagccaaatgtactttagacatgaatgcaactatttaaagaatagcttcatcaatgttattccttacatgtcataagattcttacttaaacttggtcttctttcaaattgtttgtatgaagatgctgtacccacttgaacagtcctcaggtgtttacataaatactatgttttacagttttcatattttaaaatattaataaagttaaatcacaatagt

SEQ ID NO: 82 - DNA, 4130 bp, Homo sapiens
gaagctcggtgccggctcgcgcgatcggtgggacagaatttcgttgttttcaccgacgagactggaggaaacaacaccaaatagggataccatgggacaaacagtgaatgaagattccatggatgtcaagaaggagaatcaagagaaaactcctcagtcaagtacatcttctgtacaaagggatgatttccactgggaggagtatttgaaagagactgggtctataagtgctccttcagagtgcttccgtcagtctcagattccacctgtgaatgatttcaaagttggtatgaaattggaagcccgtgaccctcgcaatgccacttcagtatgtattgctacggttattggaattactggggccaggttacggttacgactggatggtagtgacaacagaaatgatttttggaggcttgtcgattccccagacatacaacctgttgggacatgtgaaaaggaaggagacttacttcaacctccactagggtaccagatgaatacatcctcctggccgatgttcctcttaaagacactaaatgggtctgaaatggcatctgccacattatttaagaaggaaccaccaaagcccccactaaataattttaaagtggggatgaaactggaagctattgacaaaaagaacccgtatctcatctgtcctgcgaccattggagatgttaaaggggatgaagttcatatcacatttgatggctggagtggagcttttgattactggtgcaagtatgattctcgagatattttcccagctgggtggtgtcgcctgacaggagatgtattacaacccccaggaactagtgttcctattgtaaagaatatagcaaaaacagagtcttctccttccgaagcaagccagcattcaatgcagtctccacagaaaactactctaatattaccaacacagcaggtcaggagatcaagtcgaattaaaccacctggacctactgcagtccccaaaaggagcagttctgttaaaaatatcacaccaaggaaaaaaggtccaaactcaggaaaaaaggaaaaacctttgcccgtgatatgttctacatctgcagcttctctaaaatcgctgaccagagaccgtggcatgttatataaagatgtcgcttctgggccatgtaaaatagtgatgtctacagtctgtgtctatgtaaacaaacatggaaactttggccctcatctggatcccaagagaatccagcagctgcctgaccacttcggcccgggcccggtgaatgtggtgcttcgccggattgtgcaggcctgtgtggattgtgcccttgaaactaaaactgtttttggatacctgaagccagataatcgtggaggagaagtgataactgcctcctttgatggggaaactcattccatccagctccctccagtgaacagtgcatcatttgctcttcgctttcttgagaacttctgccacagtctgcagtgtgataaccttttgagtagccagccttttagttcttccaggggtcatactcacagctctgcagagcatgataaaaatcagtcagcaaaagaagatgtaacagaaaggcaaagcaccaaacgatctcctcagcaaactgtaccatatgttgttcctctctctcctaagctccccaaaacaaaggagtatgcgtctgaaggagaaccattgtttgctgggggaagtgccattcccaaagaggagaatctttcagaagattctaagagctcatcactaaattcaggaaattatttgaatcctgcctgtagaaatcctatgtatattcatacttcagtctcccaggatttttctcgaagtgtgccaggcaccacaagttcaccactagttggggacatatcccccaagagcagtccccatgaagttaaattccaaatgcagaggaaaagtgaagctccaagttatatagctgtacctgatcccagtgtcctgaaacaaggcttctctaaggacccttcaacctggtctgtggatgaagtgatacagtttatgaaacatacagatcctcagatatcaggccccctcgccgacctcttcaggcaacatgaaattgatgggaaggctctgttcctactcaagagtgatgtgatgatgaagtatatggggctgaagctggggccagcattaaagctgtgttactacattgaaaagcttaaagaaggaaaatacagttaaaaaatgtgtaagtttagattggacataattctcaggtgtactgttaacattttaatttaaaagtatttctcttagcagtttttgttttgtagacagttcccataaaaatattttatcagaattgcagaactgtagtaacagttcagtcaactttgtttttttcctggagtcaccaaccagctttgggagacacagccgccactcccccagtctacttctttaaaaagcatttaacaggttagtattggcatattcaaattggcagttctttatgtctttttaaattttcattgtacagtttacaaatatacttaatgtagttaacagagaaaaacctttgattttggttaacctttatatctagaaccaaaacagctaaatcccaaaggggaaaatatcagggattgacaacttctataattaaatccatgagaatttttcctcactagagaatttaaaggtgcacctgtagatatcatcttttctcagatattttgtgtgatactctgtggtgttctgttcatgttctatcagtatatctagaaagggaatagccatataaattattttccttttattatttctctgtatgttgtatttgatcatatttaaaggaaaaaagcaagcttataagctttcatgaagtgttcttaccagtttttgataaattttttaaattataggatagaattgtcattttatgcaggagatatttatactacgagggttgtttggatggagtcagattaaattttttcagtgaaattcctattattttaaaactttccatattttcactacgcttggacatttaactaggcattcttttcttacatctctatatgaagatacctgtgtccaaaatttttgaagatatatattgtatgtgtttattcttcatatggtactttaccatatttatatattgttttatacctgtaggtttacacacaagtaaatctttttttcttgaatttaatctgggcactttgcactgccacaggggtgacgatggaactatgtatatagttaggatgttttgatttcggtaaaaaatatatgtcccatcgttgctatcaccagtacctctcagcttactcttcaggggatatgaaacaatctgtagattgggtttccataacagggaagtccgtcctaatgccaagtttctaattaatttgcttagttctgagccatttattctgctacactttgaaagatatattagttctgacttattgttggggctttattttatttttatttttttgagatggagtttcactcttgtgcccaggctggagtgcaatagctcgatctcggctcactgcaacctcgcctccctggttcaagcaattctcctgcctcagcgattacaggaatgcaccaccacgcccagctaattttgtagagatggggtttctccatgttggtcaggctggtctcaaactcctgacctcaggtgatccgcccacctcggccttgcaaaatgctgggattacaggcatgagccacctgcccagccatgtttggggctttattttataagttagaactttgaagaggaaatggtgctatatgtttattgttattactttgtgtaactttgatgtaatgtttataagctatgagaatcagttataaaagtattagccatttgttgtaaatgccaataaaatattcaccaggggcaagaatgtacattttctttttagaaaaccaaatgtactttagacatgaatgcaactatttaaagaatagcttcatttatgttattccttacatgtcataagattcttacttaaacttggtcttctttcaagttgtttgtatgaagatgctgtacccacttgaacagtcctcaggtgtttacataaatactatgttttacagttttcatattttaaaatattaataaagttaatcgcaacgattc

SEQ ID NO: 83 - DNA, 1529 bp, Homo sapiens
ggctgctgtttctaaaacccctttccctctaacccacaccacctttctactcactgatgccttcaggaagccataatggatggcacagagattgctgtttcccctcggtcactgcattcagaactcatgtgccctatctgcctggacatgctgaagaatacgatgaccaccaaggagtgcctccacagattctgctctgactgcattgtcacagccctacggagcgggaacaaggagtgtcctacctgccgaaagaagctggtgtccaagcgatccctacggccagaccccaactttgatgccctgatctctaagatctatcctagccgggaggaatacgaggcccatcaagaccgagtgcttatccgcctgagccgcctgcacaaccagcaggcattgagctccagcattgaggaggggctacgcatgcaggccatgcacagggcccagcgtgtgaggcggccgataccagggtcagatcagaccacaacgatgagtgggggggaaggagagcccggggagggagaaggggatggagaagatgtgagctcagactccgcccctgactctgccccaggccctgctcccaagcgaccccgtggagggggcgcaggggggagcagtgtagggacagggggaggcggcactggtggggtgggtgggggtgccggttcggaagactctggtgaccggggagggactctgggagggggaacgctgggccccccaagccctcctggggcccccagccccccagagccaggtggagaaattgagctcgtgttccggccccaccccctgctcgtggagaagggagaatactgccagacgaggtatgtgaagacaactgggaatgccacagtggaccacctctccaagtacttggccctgcgcattgccctcgagcggaggcaacagcaggaagcaggggagccaggagggcctggagggggcgcctctgacaccggaggacctgatgggtgtggcggggagggtgggggtgccggaggaggtgacggtcctgaggagcctgctttgcccagcctggagggcgtcagtgaaaagcagtacaccatctacatcgcacctggaggcggggcgttcacgacgttgaatggctcgctgaccctggagctggtgaatgagaaattctggaaggtgtcccggccactggagctgtgctatgctcccaccaaggatccaaagtgaccccaccaggggacagccagaggaaggggaccatggggtatccctgtgtcctggtctatcaccccagcttctttgtcccccagtacccccagcccagccagccaataagaggacacaaatgaggacacgtggcttttatacaaagtatctatatgagattcttctatattgtacagagtggggcaaaacacgcccccatctgctgccttttctattgccctgcaacgtcccatctatacgaggtgttggagaaggtgaagaaccctcccattcacgcccgcctaccaacaacaaacgtgcttttttcctctttgaaaaaa

SEQ ID NO: 84 - DNA, 1036 bp, Homo sapiens
cccggacggcgtttctcctccgagcggcgccggtttcggcttggggggggcggggtacagcccatccatgaccatgggcgacaagaagagcccgaccaggccaaaaagacaagcgaaacctgccgcagacgaagggttttgggattgtagcgtctgcaccttcagaaacagtgctgaagcctttaaatgcagcatctgcgatgtgaggaaaggcacctccaccagaaaacctcggatcaattctcagctggtggcacaacaagtggcacaacagtatgccaccccaccaccccctaaaaaggagaagaaggagaaagttgaaaagcaggacaaagagaaacctgagaaagacaaggaaattagtcctagtgttaccaagaaaaataccaacaagaaaaccaaaccaaagtctgacattctgaaagatcctcctagtgaagcaaacagcatacagtctgcaaatgctacaacaaagaccagcgaaacaaatcacacctcaaggccccggctgaaaaacgtggacaggagcactgcacagcagttggcagtaactgtgggcaacgtcaccgtcattatcacagactttaaggaaaagactcgctcctcatcgacatcctcatccacagtgacctccagtgcagggtcagaacagcagaaccagagcagctcggggtcagagagcacagacaagggctcctcccgttcctccacgccaaagggcgacatgtcagcagtcaatgatgaatctttctgaaattgcacatggaattgtgaaaactatgaatcagggtatgaaattcaaaacctccacctgcccatgctgcttgcatccctggagaatcttctgtggacatcgacctcttagtgatgctgccaggataatttctgcttgccatgggcatctggccaccaaggaatttcgcaccctgacgattactcttgacacttttatgtattccattgttttatatgattttcctaacaatcatttataattggatgtgctcctgaatctacttttaataaaaaaaaaaaa

SEQ ID NO: 85 - DNA, 11910 bp, Homo sapiens
  misc_feature (89)..(89): unknown nucleotide
atggcgcacagctgtcggtggcgcttccccgcccgacccgggaccaccgggggcggcggcggcggggggcgccggggcctagggggcgncccgcggcaacgcgtcccggccctgctgcttccccccgggcccccggtcggcggtggcggccccggggcgcccccctcccccccggctgtggcggccgcggcggcggcggcgggaagcagcggggctggggttccagggggagcggccgccgcctcagcagcctcctcgtcgtccgcctcgtcttcgtcttcgtcatcgtcctcagcctcttcagggccggccctgctccgggtgggcccgggcttcgacgcggcgctgcaggtctcggccgccatcggcaccaacctgcgccggttccgggccgtgtttggggagagcggcgggggaggcggcagcggagaggatgagcaattcttaggttttggctcagatgaagaagtcagagtgcgaagtcccacaaggtctccttcagttaaaactagtcctcgaaaacctcgtgggagacctagaagtggctctgaccgaaattcagctatcctctcagatccatctgtgttttcccctctaaataaatcagagaccaaatctggagataagatcaagaagaaagattctaaaagtatagaaaagaagagaggaagacctcccaccttccctggagtaaaaatcaaaataacacatggaaaggacatttcagagttaccaaagggaaacaaagaagatagcctgaaaaaaattaaaaggacaccttctgctacgtttcagcaagccacaaagattaaaaaattaagagcaggtaaactctctcctctcaagtctaagtttaagacagggaagcttcaaataggaaggaagggggtacaaattgtacgacggagaggaaggcctccatcaacagaaaggataaagaccccttcggtctcctcattaattctgaactggaaaagccccagaaagtccggaaagacaaggaaggaacacctccacttacaaaagaagataagacagttgtcagacaaagccctcgaaggattaagccagttaggattattccttcttcaaaaaggacagatgcaaccattgctaagcaactcttacagagggcaaaaaaagggggctcaaaagaaaattgaaaaagaagcagctcagctgcagggaagaaaggtgaagacacaggtcaaaaatattcgacagttcatcatgcctgttgtcagtgctatctcctcgcggatcattaagacccctcggcggtttatagaggatgaggattatgaccctccaattaaaattgcccgattagagtctacaccgaatagtagattcagtgccccgtcctgtggatcttctgaaaaatcaagtgcagcttctcagcactcctctcaaatgtcttcagactcctctcgatctagtagccccagtgttgatacctccacagactctcaggcttctgaggagattcaggtacttcctgaggagcggagcgatacccctgaagttcatcctccactgcccatttcccagtccccagaaaatgagagtaatgataggagaagcagaaggtattcagtgtcggagagaagttttggatctagaacgacgaaaaaattatcaactctacaaagtgccccccagcagcagacctcctcgtctccacctccacctctgctgactccaccgccaccactgcagccagcctccagtatctctgaccacacaccttggcttatgcctccaacaatccccttagcatcaccatttttgcctgcttccactgctcctatgcaagggaagcgaaaatctattttgcgagaaccgacatttaggtggacttctttaaagcattctaggtcagagccacaatacttttcctcagcaaagtatgccaaagaaggtcttattcgcaaaccaatatttgataatttccgaccccctccactaactcccgaggacgttggctttgcatctggtttttctgcatctggtaccgctgcttcagcccgattgttttcgccactccattctggaacaaggtttgatatgcacaaaaggagccctcttctgagagctccaagatttactccaagtgaggctcactctagaatatttgagtctgtaaccttgcctagtaatcgaacttctgctggaacatcttcttcaggagtatccaatagaaaaaggaaaagaaaagtgtttagtcctattcgatctgaaccaagatctccttctcactccatgaggacaagaagtggaaggcttagtagttctgagctctcacctctcacccccccgtcttctgtctcttcctcgttaagcatttctgttagtcctcttgccactagtgccttaaacccaacttttacttttccttctcattccctgactcagtctggggaatctgcagagaaaaatcagagaccaaggaagcagactagtgctccggcagagccattttcatcaagtagtcctactcctctcttcccttggtttaccccaggctctcagactgaaagagggagaaataaagacaaggcccccgaggagctgtccaaagatcgagatgctgacaagagcgtggagaaggacaagagtagagagagagaccgggagagagaaaaggagaataagcgggagtcaaggaaagagaaaaggaaaaagggatcagaaattcagagtagttctgctttgtatcctgtgggtagggtttccaaagagaaggttgttggtgaagatgttgccacttcatcttctgccaaaaaagcaacagggcggaagaagtcttcatcacatgattctgggactgatattacttctgtgactcttggggatacaacagctgtcaaaaccaaaatacttataaagaaagggagaggaaatctggaaaaaaccaacttggacctcggcccaactgccccatccctggagaaggagaaaaccctctgcctttccactccttcatctagcactgttaaacattccacttcctccataggctccatgttggctcaggcagacaagcttccaatgactgacaagagggttgccagcctcctaaaaaaggccaaagctcagctctgcaagattgagaagagtaagagtcttaaacaaaccgaccagcccaaagcacagggtcaagaaagtgactcatcagagacctctgtgcgaggaccccggattaaacatgtctgcagaagagcagctgttgcccttggccgaaaacgagctgtgtttcctgatgacatgcccaccctgagtgccttaccatgggaagaacgagaaaagattttgtcttccatggggaatgatgacaagtcatcaattgctggctcagaagatgctgaacctcttgctccacccatcaaaccaattaaacctgtcactagaaacaaggcaccccaggaacctccagtaaagaaaggacgtcgatcgaggcggtgtgggcagtgtcccggctgccaggtgcctgaggactgtggtgtttgtactaattgcttagataagcccaagtttggtggtcgcaatataaagaagcagtgctgcaagatgagaaaatgtcagaatctacaatggatgccttccaaagcctacctgcagaagcaagctaaagctgtgaaaaagaaagagaaaaagtctaagaccagtgaaaagaaagacagcaaagagagcagtgttgtgaagaacgtggtggactctagtcagaaacctaccccatcagcaagagaggatcctgccccaaagaaaagcagtagtgagcctcctccacgaaagcccgtcgaggaaaagagtgaagaagggaatgtctcggcccctgggcctgaatccaaacaggccaccactccagcttccaggaagtcaagcaagcaggtctcccagccagcactggtcatcccgcctcagccacctactacaggaccgccaagaaaagaagttcccaaaaccactcctagtgagcccaagaaaaagcagcctccaccaccagaatcaggtccagagcagagcaaacagaaaaaagtggctccccgcccaagtatccctgtaaaacaaaaaccaaaagaaaaggaaaaaccacctccggtcaataagcaggagaatgcaggcactttgaacatcctcagcactctctccaatggcaatagttctaagcaaaaaattccagcagatggagtccacaggatcagagtggactttaaggaggattgtgaagcagaaaatgtgtgggagatgggaggcttaggaatcttgacttctgttcctataacacccagggtggtttgctttctctgtgccagtagtgggcatgtagagtttgtgtattgccaagtctgttgtgagcccttccacaagttttgtttagaggagaacgagcgccctctggaggaccagctggaaaattggtgttgtcgtcgttgcaaattctgtcacgtttgtggaaggcaacatcaggctacaaagcagctgctggagtgtaataagtgccgaaacagctatcaccctgagtgcctgggaccaaactaccccaccaaacccacaaagaagaagaaagtctggatctgtaccaagtgtgttcgctgtaagagctgtggatccacaactccaggcaaagggtgggatgcacagtggtctcatgatttctcactgtgtcatgattgcgccaagctctttgctaaaggaaacttctgccctctctgtgacaaatgttatgatgatgatgactatgagagtaagatgatgcaatgtggaaagtgtgatcgctgggtccattccaaatgtgagaatctttcagatgagatgtatgagattctatctaatctgccagaaagtgtggcctacacttgtgtgaactgtactgagcggcaccctgcagagtggcgactggcccttgaaaaagagctgcagatttctctgaagcaagttctgacagctttgttgaattctcggactaccagccatttgctacgctaccggcaggctgccaagcctccagacttaaatcccgagacagaggagagtataccttcccgcagctcccccgaaggacctgatccaccagttcttactgaggtcagcaaacaggatgatcagcagcctttagatctagaaggagtcaagaggaagatggaccaagggaattacacatctgtgttggagttcagtgatgatattgtgaagatcattcaagcagccattaattcagatggaggacagccagaaattaaaaaagccaacagcatggtcaagtccttcttcattcggcaaatggaacgtgtttttccatggttcagtgtcaaaaagtccaggttttgggagccaaataaagtatcaagcaacagtgggatgttaccaaacgcagtgcttccaccttcacttgaccataattatgctcagtggcaggagcgagaggaaaacagccacactgagcagcctcctttaatgaagaaaatcattccagctcccaaacccaaaggtcctggagaaccagactcaccaactcctctgcatcctcctacaccaccaattttgagtactgataggagtcgagaagacagtccagagctgaacccacccccaggcatagaagacaatagacagtgtgcgttatgtttgacttatggtgatgacagtgctaatgatgctggtcgtttactatatattggccaaaatgagtggacacatgtaaattgtgctttgtggtcagcggaagtgtttgaagatgatgacggatcactaaagaatgtgcatatggctgtgatcaggggcaagcagctgagatgtgaattctgccaaaagccaggagccaccgtgggttgctgtctcacatcctgcaccagcaactatcacttcatgtgttcccgagccaagaactgtgtctttctggatgataaaaaagtatattgccaacgacatcgggatttgatcaaaggcgaagtggttcctgagaatggatttgaagttttcagaagagtgtttgtggactttgaaggaatcagcttgagaaggaagtttctcaatggcttggaaccagaaaatatccacatgatgattgggtctatgacaatcgactgcttaggaattctaaatgatctctccgactgtgaagataagctctttcctattggatatcagtgttccagggtatactggagcaccacagatgctcgcaagcgctgtgtatatacatgcaagatagtggagtgccgtcctccagtcgtagagccggatatcaacagcactgttgaacatgatgaaaacaggaccattgcccatagtccaacatcttttacagaaagttcatcaaaagagagtcaaaacacagctgaaattataagtcctccatcaccagaccgacctcctcattcacaaacctctggctcctgttattatcatgtcatctcaaaggtccccaggattcgaacacccagttattctccaacacagagatcccctggctgtcgaccgttgccttctgcaggaagtcctaccccaaccactcatgaaatagtcacagtaggtgatcctttactctcctctggacttcgaagcattggctccaggcgtcacagtacctcttccttatcaccccagcggtccaaactccggataatgtctccaatgagaactgggaatacttactctaggaataatgtttcctcagtctccaccaccgggaccgctactgatcttgaatcaagtgccaaagtagttgatcatgtcttagggccactgaattcaagtactagtttagggcaaaacacttccacctcttcaaatttgcaaaggacagtggttactgtaggcaataaaaacagtcacttggatggatcttcatcttcagaaatgaagcagtccagtgcttcagacttggtgtccaagagctcctctttaaagggagagaagaccaaagtgctgagttccaagagctcagagggatctgcacataatgtggcttaccctggaattcctaaactggccccacaggttcataacacaacatctagagaactgaatgttagtaaaatcggctcctttgctgaaccctcttcagtgtcgttttcttctaaagaggccctctccttcccacacctccatttgagagggcaaaggaatgatcgagaccaacacacagattctacccaatcagcaaactcctctccagatgaagatactgaagtcaaaaccttgaagctatctggaatgagcaacagatcatccattatcaacgaacatatgggatctagttccagagataggagacagaaagggaaaaaatcctgtaaagaaactttcaaagaaaagcattccagtaaatcttttttggaacctggtcaggtgacaactggtgaggaaggaaacttgaagccagagtttatggatgaggttttgactcctgagtatatgggccaacgaccatgtaacaatgtttcttctgataagattggtgataaaggcctttctatgccaggagtccccaaagctccacccatgcaagtagaaggatctgccaaggaattacaggcaccacggaaacgcacagtcaaagtgacactgacacctctaaaaatggaaaatgagagtcaatccaaaaatgccctgaaagaaagtagtcctgcttcccctttgcaaatagagtcaacatctcccacagaaccaatttcagcctctgaaaatccaggagatggtccagtggcccaaccaagccccaataatacctcatgccaggattctcaaagtaacaactatcagaatcttccagtacaggacagaaacctaatgcttccagatggccccaaacctcaggaggatggctcttttaaaaggaggtatccccgtcgcagtgcccgtgcacgttctaacatgttttttgggcttaccccactctatggagtaagatcctatggtgaagaagacattccattctacagcagctcaactgggaagaagcgaggcaagagatcagctgaaggacaggtggatggggccgatgacttaagcacttcagatgaagacgacttatactattacaacttcactagaacagtgatttcttcaggtggagaggaacgactggcatcccataatttatttcgggaggaggaacagtgtgatcttccaaaaatctcacagttggatggtgttgatgatgggacagagagtgatactagtgtcacagccacaacaaggaaaagcagccagattccaaaaagaaatggtaaagaaaatggaacagagaacttaaagattgatagacctgaagatgctggggagaaagaacatgtcactaagagttctgttggccacaaaaatgagccaaagatggataactgccattctgtaagcagagttaaaacacagggacaagattccttggaagctcagctcagctcattggagtcaagccgcagagtccacacaagtaccccctccgacaaaaatttactggacacctataatactgagctcctgaaatcagattcagacaataacaacagtgatgactgtgggaatatcctgccttcagacattatggactttgtactaaagaatactccatccatgcaggctttgggtgagagcccagagtcatcttcatcagaactcctgaatcttggtgaaggattgggtcttgacagtaatcgtgaaaaagacatgggtctttttgaagtattttctcagcagctgcctacaacagaacctgtggatagtagtgtctcttcctctatctcagcagaggaacagtttgagttgcctctagagctaccatctgatctgtctgtcttgaccacccggagtcccactgtccccagccagaatcccagtagactagctgttatctcagactcaggggagaagagagtaaccatcacagaaaaatctgtagcctcctctgaaagtgacccagcactgctgagcccaggagtagatccaactcctgaaggccacatgactcctgatcattttatccaaggacacatggatgcagaccacatctctagccctccttgtggttcagtagagcaaggtcatggcaacaatcaggatttaactaggaacagtagcacccctggccttcaggtacctgtttccccaactgttcccatccagaaccagaagtatgtgcccaattctactgatagtcctggcccgtctcagatttccaatgcagctgtccagaccactccaccccacctgaagccagccactgagaaactcatagttgttaaccagaacatgcagccactttatgttctccaaactcttccaaatggagtgacccaaaaaatccaattgacctcttctgttagttctacacccagtgtgatggagacaaatacttcagtattgggacccatgggaggtggtctcacccttaccacaggactaaatccaagcttgccaacttctcaatctttgttcccttctgctagcaaaggattgctacccatgtctcatcaccagcacttacattccttccctgcagctactcaaagtagtttcccaccaaacatcagcaatcctccttcaggcctgcttattggggttcagcctcctccggatccccaacttttggtttcagaatccagccagaggacagacctcagtaccacagtagccactccatcctctggactcaagaaaagacccatatctcgtctacagacccgaaagaataaaaaacttgctccctctagtaccccttcaaacattgccccttctgatgtggtttctaatatgacattgattaacttcacaccctcccagcttcctaatcatccaagtctgttagatttggggtcacttaatacttcatctcaccgaactgtccccaacatcataaaaagatctaaatctagcatcatgtattttgaaccggcacccctgttaccacagagtgtgggaggaactgctgccacagcggcaggcacatcaacaataagccaggatactagccacctcacatcagggtctgtgtctggcttggcatccagttcctctgtcttgaatgttgtatccatgcaaactaccacaacccctacaagtagtgcgtcagttccaggacacgtcaccttaaccaacccaaggttgcttggtaccccagatattggctcaataagcaatcttttaatcaaagctagccagcagagcctggggattcaggaccagcctgtggctttaccgccaagttcaggaatgtttccacaactggggacatcacagaccccctctactgctgcaataacagcggcatctagcatctgtgtgctcccctccactcagactacgggcataacagccgcttcaccttctggggaagcagacgaacactatcagcttcagcatgtgaaccagctccttgccagcaaaactgggattcattcttcccagcgtgatcttgattctgcttcagggccccaggtatccaactttacccagacggtagacgctcctaatagcatgggactggagcagaacaaggctttatcctcagctgtgcaagccagccccacctctcctgggggttctccatcctctccatcttctggacagcggtcagcaagcccttcagtgccgggtcccactaaacccaaaccaaaaaccaaacggtttcagctgcctctagacaaagggaatggcaagaagcacaaagtttcccatttgcggaccagttcttctgaagcacacattccagaccaagaaacgacatccctgacctcaggcacagggactccaggagcagaggctgagcagcaggatacagctagcgtggagcagtcctcccagaaggagtgtgggcaacctgcagggcaagtcgctgttcttccggaagttcaggtgacccaaaatccagcaaatgaacaagaaagtgcagaacctaaaacagtggaagaagaggaaagtaatttcagctccccactgatgctttggcttcagcaagaacaaaagcggaaggaaagcattactgagaaaaaacccaagaaaggacttgtttttgaaatttccagtgatgatggctttcagatctgtgcagaaagtattgaagatgcctggaagtcattgacagataaagtccaggaagctcgatcaaatgcccgcctaaagcagctctcatttgcaggtgttaacggtttgaggatgctggggattctccatgatgcagttgtgttcctcattgagcagctgtctggtgccaagcactgtcgaaattacaaattccgtttccacaagccagaggaggccaatgaaccccccttgaaccctcacggctcagccagggctgaagtccacctcaggaagtcagcatttgacatgtttaacttcctggcttctaaacatcgtcagcctcctgaatacaaccccaatgatgaagaagaggaggaggtacagctgaagtcagctcggagggcaactagcatggatctgccaatgcccatgcgcttccggcacttaaaaaagacttctaaggaggcagttggtgtctacaggtctcccatccatggccggggtcttttctgtaagagaaacattgatgcaggtgagatggtgattgagtatgccggcaacgtcatccgctccatccagactgacaagcgggaaaagtattacgacagcaagggcattggttgctatatgttccgaattgatgactcagaggtagtggatgccaccatgcatggaaatcgtgcacgcttcatcaatcactcgtgtgagcctaactgctattctcgggtcatcaatattgatgggcagaagcacattgtcatctttgccatgcgtaagatctaccgaggagaggaactcacttacgactataagttccccattgaggatgccagcaacaagctgccctgcaactgtggcgccaagaaatgccggaagttcctaaactaa

SEQ ID NO: 86 - DNA, 1680 bp, Homo sapiens
atggacaatcagtgcaccgtccaggtgaggttagagctggggcatcgcgcccaactgcgcaagaagcccaccacggaggggttcactcacgactggatggtgtttgtccgcggccccgagcaatgtgacatccagcacttcgtggagaaggtggtcttctggctgcacgacagcttccccaagcccagacgcgtgtgcaaggagcccccctacaaagtagaggagtcggggtacgctggcttcatcatgcccatcgaggtgcacttcaaaaacaaggaggagccgaggaaggtctgcttcacctacgacctgttcctgaacctggaaggcaacccgcccgtgaaccacctgcgctgcgagaagctcaccttcaacaaccccaccacggagttccggtacaagctcctgcgggccggcggggtgatggtaatgcccgaaggagcagacacggtgtccaggcccagtcccgactaccccatgttacccacaattccactctctgccttctctgaccccaagaagaccaaaccatcccacggctccaaggacgccaacaaggagagcagcaagacctccaagccacacaaggtgaccaaggagcaccgggagcgcccccgcaaagactccgagagcaagagctcctccaaggagctggagcgtgagcaggccaaaagctccaaggacacctcgcggaagctgggcgagggccggctgcccaaggaggagaaggcgccaccgcccaaggctgccttcaaggaacccaagatggccctgaaagagaccaagctggaaagcacgtcccccaaccctgggcccccacccccacccccacccccaccccgggcttccagcaagcggccggccaccgccgactcgccaaagcccagcgccaagaagcagaagaagagcagctcgaaggggtcccggagtgctccaggcacctcgccccgcacctcctcctcctcctccttctcggacaagaagccggccaaggacaagagcagcaccagaggggagaaggtgaaggccgagagtgagccccgggaggccaaaaaggccctggaggtggaggagtccaactcagaggacgaggcctccttcaagtccgagtctgcccagtcaagcccgtccaactccagctccagctcagactccagctcagactcagacttcgagccatcccagaaccacagccaaggacccctgcgctccatggtggaggacctgcagtccgaggagtccgacgaggacgactcttcgtcaggcgaggaggctgccggcaagaccaacccggggagggactccaggttgagcttcagcgacagcgagagtgacaacagcgccgactcctccctgcccagccgtgagcccccacccccccagaagccacccccgcccaacagcaaggtgtcaggccggaggagccccgagtcctgcagcaagcctgagaagatcctcaagaagggcacctacgacaaggcctacacggatgagctggtggagctacaccggaggctgatggcgctgcgggagcgcaacgtgctgcagcagattgtgaatctgatcgaggagactggccacttcaatgtcaccaacaccaccttcgacttcgacctcttctccctggacgagaccaccgtgcgcaaactgcagagctgcctggaggccgtggccacatga

SEQ ID NO: 87 - DNA, 3171 bp, Homo sapiens
gggacagcttagggactatcgtcctgggactagggggaagttcgcgactttctgaagactggcaggaatgtgcctcctggccctcgatgcttcccccctgaggggaggcatcgtgagggactgtggcaggcttcactgaacgctgagccggggaggtccaactccacgtatggatccggggaatgagaattcagccacagaaggccgccgcgatcatagacctagatcccgacttcgaaccccagagccgtccccgctcctgcacctggccccttccccgaccagagatcgctaaccagccgtccgagccgcccgaggtggagccagatctgggggaaaaggtacacacggaggggcgctcagagccgatcctgttgccctctcggctctcagagccggccgggggcccccagcccggaatcctgggggctgtaacaggtcctcggaagggaggctcccgccggaatgcctggggaaatcagtcatatgcagaattcatcagccaggccattgaaagcgccccggagaagcgactgacacttgcccagatttacgagtggatggtccgtactgtaccctacttcaaggacaagggtgacagcaacagctcagcaggatggaagaactcgatccgccacaacctgtccctgcacagcaagttcatcaaggttcacaacgaggccaccggcaaaagctcttggtggatgctgaaccctgagggaggcaagagcggcaaagccccccgccgccgggccgcctccatggatagcagcagcaagctgctccggggccgcagtaaagcccccaagaagaaaccatctgtgctgccagctccacccgaaggtgccactccaacgagccctgtcggccactttgccaagtggtcaggcagcccttgctctcgaaaccgtgaagaagccgatatgtggaccaccttccgtccacgaagcagttcaaatgccagcagtgtcagcacccggctgtcccccttgaggccagagtctgaggtgctggcggaggaaataccagcttcagtcagcagttatgcagggggtgtccctcccaccctcaatgaaggtctagagctgttagatgggctcaatctcacctcttcccattccctgctatctcggagtggtctctctggcttctctttgcagcatcctggggttaccggccccttacacacctacagcagctcccttttcagcccagcagaggggcccctgtcagcaggagaagggtgcttctccagctcccaggctctggaggccctgctcacctctgatacgccaccaccccctgctgacgtcctcatgacccaggtagatcccattctgtcccaggctccgactcttctgttgctgggggggcttccttcctccagtaagctggccacgggcgtcggcctgtgtcccaagcccctagaggctcgaggccccagcagtctggttcccaccctttctatgatagcaccacctccagtcatggcaagtgcccccatccccaaggctctggggactcctgtgctcacaccccctactgaagctgcaagccaagacagaatgcctcaggatctagatcttgatatgtatatggagaacctggagtgtgacatggataacatcatcagtgacctcatggatgagggcgagggactggacttcaactttgagccagatccctgagtcatgcctggaagctttgtcccctgcttcagatgtggagccaggcgtgttcatatctactctttacccttgagccctccccaggaatttgggaccctgctttagagctagggtggggtctggtcacacacaggtgttgaagaaattataaagataaagctgccccatctggggacgatatggggagggagatgggaggggaaaggggagagggtttttctcactgtgccaattagggggtaaggccccctctcaggagccatcatcggctttccccattcctacccacttaggctttgtagcaagatgagcaatgctgttggaaatgtgaagtcaccagtggccttacccctgcctttgggagcaggatttttttgtagagagtcttatctgagctgagccaggctagctggagcctgggatttctatgcagtggccccttaggccagtgatgtgcggtgggtgggctgtttaggggatctggaagggccaaggtctgagcactggagtggctcgccaggccaaatcacccttagaaggctgcagataacagaaaggctttttataaacttttaaagaaatataaacacaaatatagagattttttaaccatggcagggtgctagtggtgggcagaatgcttttttttctttctgaaggctttgtgatagtgacatgatacaaacactacagacaataaatattaggagacacagggaagtggggagaggtggggagtaatagtaaacacagggaagagctcccctacggaccaggtatagagaaaggtctatgcagaaataggttagagtttccctaacaaaaaagctaacccaggtcccctcattccttcaacttgtgcctgggagtgtgtggtgttagggtgcagccacactcttctatgacccagcatgggttagtgctatggtgggagagtacattgaaggcctggaattagcttggggccagggaagggactgggaggggagagaagagaaggagggaaggatttaggatggtaaagttaggtacagagacctccctgttcaaggcccctgacagctgtccctgcccttcttccccttccctgactgcaggggttatgtggaagtgtgtgtggcagcaggcagcggggaggggaggaacagggaagggggagctggggagcttggctgagggtctgggaaatgagcagggatggggggggatgtggatcaggtttactagcacctgccagggaggccatctggggctccttctccaccccagcccccaaagcagcccttcccccagtgccctttgcatcgtcccctcccccacccctgctgtgggttcccatcatttcctgtgtcagcgcctggcctacccagattgtatcatgtgctagattggagtggggaagtgtgtcaaatcaataaatgaataaattcaataaatgcctataaccagcagaaaaaaaaaaaaaaaaaaaaaaaaaaaaaaa

SEQ ID NO: 88 - DNA, 3282 bp, Homo sapiens
atgaaggagatggtaggaggctgctgcgtatgttcggacgagaggggctgggccgagaacccgctggtctactgcgatgggcacgcgtgcagcgtggccgtccaccaagcttgctatggcatcgttcaggtgccaacgggaccctggttctgccggaaatgtgaatctcaggagcgagcagccagggtgaggtgtgagctgtgcccacacaaagacggggcattgaagaggactgataatggaggctgggcacacgtggtgtgtgccctctacatccccgaggtgcaatttgccaacgtgctcaccatggagcccatcgtgctgcagtacgtgcctcatgatcgcttcaacaagacctgttacatctgcgaggagacgggccgggagagcaaggcggcctcgggagcctgcatgacctgtaaccgccatggatgtcgacaagctttccacgtcacctgtgcccaaatggcaggcttgctgtgtgaggaagaagtgctggaggtggacaacgtcaagtactgcggctactgcaaataccacttcagcaagatgaagacatcccggcacagcagcgggggaggcggaggaggcgctggaggaggaggtggcagcatggggggaggtggcagtggtttcatctctgggaggagaagccggtcagcctcaccatccacgcagcaggagaagcaccccacccaccacgagaggggccagaagaagagtcgaaaggacaaagaacgccttaagcagaagcacaagaagcggcctgagtcgccccccagcatcctcaccccgcccgtggtccccactgctgacaaggtctcctcctcggcttcctcttcctcccaccacgaggccagcacgcaggagacctctgagagcagcagggagtcaaaggggaaaaagtcttccagccatagcctgagtcataaagggaagaaactgagcagtgggaaaggtgtgagcagttttacctccgcctcctcttcttcctcctcctcttcctcctcctctggggggcccttccagcctgcagtctcgtccctgcagagctcccctgacttctctgcattccccaagctggagcagccagaggaggacaagtactccaagcccacagcccccgccccttcagcccctccttctccctcagctcccgagccccccaaggctgacctttttgagcagaaggtggtcttctctggctttgggcccatcatgcgcttctccaccaccacctccagctcaggccgggcccgggcgccctcccctggggactataagtctccccacgtcacggggtctggggcctcggcaggcacccacaaacggatgcccgcactgagtgccacccctgtgcctgctgatgagacccctgagacaggcctgaaggagaagaagcacaaagccagcaagaggagccgccatgggccaggccgtcccaagggcagccggaacaaggagggcactgggggcccagctgccccatccttgcccagtgcccagctggctggctttaccgccactgctgcctcacccttctctggaggttccctggtcagctccggcctgggaggtctgtcctcccgaacctttgggccttctgggagcttgcccagcttgagcctggagtcccccttactaggggcaggcatctacaccagtaataaggaccccatctcccacagtggcgggatgctgcgggctgtctgcagcacccctctctcctccagcctcctggggcccccagggacctcggccctgccccgcctcagccgctccccgttcaccagcaccctcccctcctcttctgcttctatctccaccactcaggtgttttctctggctggctctacctttagcctcccttctacccacatctttggaacccccatgggtgccgttaatcccctcctctcccaagctgagagcagccacacagagccagacctggaggactgcagcttccggtgtcgggggacctcccctcaggagagtctgtcttccatgtcccccatcagcagcctccccgcactcttcgaccagacagcctctgcaccctgtgggggcggccagttagacccggcggccccagggacgactaacatggagcagcttctggagaagcagggcgacggggaggccggcgtcaacatcgtggagatgctgaaggcgctgcacgcgctgcagaaggagaaccagcggctgcaagagcagatcctgagcctgacggccaaaaaggagcggctgcagattctcaacgtgcagctctctgtgcccttccctgccctgcctgctgccctgcctgccgccaacggccctgtccctgggccctatggcctgcctccccaagccgggagcagcgactccttgagcaccagcaagagccctccgggaaagagcagcctcggcctggacaactcgctgtccacttcttctgaggacccacactcaggctgcccgagccgcagcagctcgtcgctgtccttccacagcacgcccccaccgctgcccctcctccagcagagccctgccactctgcccctggccctgcctggggcccctgccccactcccgccccagccgcagaacgggttgggccgggcacccggggcagcggggctgggggccatgcccatggctgaggggctgttgggggggctggcaggcagtgggggcctgcccctcaatgggctccttggggggttgaatggggccgctgcccccaaccccgcaagcttgagccaggctggcggggcccccacgctgcagctgccaggctgtctcaacagccttacagagcagcagagacatctccttcagcagcaagagcagcagctccagcaactccagcagctcctggcctccccgcagctgaccccggaacaccagactgttgtctaccagatgatccagcagatccagcagaaacgggagctgcagcgtctgcagatggctgggggctcccagctgcccatggccagcctgctggcaggaagctccaccccgctgctgtctgcgggtacccctggcctgctgcccacagcgtctgctccacccctgctgcccgctggagccctagtggctccctcgcttggcaacaacacaagtctcatggccgcagcagctgcagctgcagcagtagcagcagcaggcggacctccagtcctcactgcccagaccaaccccttcctcagcctgtcgggagcagagggcagtggcggtggccccaaaggagggaccgctgacaaaggagcctcagccaaccaggaaaaaggctaa

SEQ ID NO: 89 - DNA, 4839 bp, Homo sapiens
atgtcggcgggcggccgtgacgaggagcggcggaagctggccgacatcatccaccactggaacgccaaccggctggacctgttcgagatcagccagccgaccgaggatttggagttccatggagtgatgagattttattttcaagataaagctgctggaaactttgcaacaaaatgtattcgggtctctagtactgccaccactcaagatgtaatcgaaacgctcgcggagaaatttcgacctgatatgcgaatgctgtcctctcccaagtattcactctatgaagtgcatgtcagcggagaaagaagattggatatagatgagaaacctctagttgtacaactgaattggaacaaagatgatcgggaaggcagatttgttcttaagaatgagaatgacgccattcctcctaaggctcaaagtaatggacctgaaaagcaggaaaaagaaggggttatccagaacttcaagagaactctctcaaagaaagaaaagaaggaaaaaaagaagagagaaaaagaggcattgcgacaggcatctgataaagatgatagacctttccaaggggaggatgttgaaaattctcgactggctgctgaggtttacaaagacatgccggaaaccagctttactcgaaccatttctaatcctgaggtggttatgaaacgacggaggcagcaaaaattggaaaagagaatgcaggaatttcggagctcagatgggcggcctgattcaggtggaacattgagaatttatgcagatagtttaaaaccaaatattccctacaagacaatcctgctgtctactacagatcctgcagactttgctgtggctgaagctttagagaagtatggtctggaaaaagaaaaccctaaggattactgcatcgcccgggttatgcttcctcctggagcccagcattctgatgaaaagggtgctaaagaaattattcttgatgatgatgagtgtcctttacaaatcttcagggaatggccaagtgacaaagggattttagtctttcagttgaagaggaggccaccagaccacatcccaaagaaaaccaagaaacacttggaaggcaagacacccaagggaaaggagagagctgacgggtctgtctatggctccacccttcctccggagaagctgccctatttagtagagttaagcccagatggttctgactctagagataagccaaagctttaccgccttcagttaagtgttactgaagttgggacagaaaagttggatgacaactctatccagttgtttggcccaggaattcagccccatcactgtgaccttaccaacatggatggagtggtcactgtgacgcccagaagtatggacgcagaaacctacgtggaaggccagcgcatctcagaaaccaccatgctgcagagtggcatgaaagtgcagtttggggcgtcccatgtatttaagtttgtggaccccagtcaggatcatgctcttgcaaaaagatctgtggatggaggcctgatggttaagggcccaagacataaacctggaattgttcaggagacaacttttgatttgggaggagatattcatagtgggacagcattaccgacaagcaagagcaccactaggctggacagcgacagagtgtcgtctgcctctagcacagccgagcggggaatggtgaagccgatgatcagagtagaacagcagccagattatcgcaggcaagaaagcagaacacaggatgcttctgggcctgagctgatactacctgcaagcattgaattcagggaaagttctgaagattcatttttgtctgccattataaattatactaatagctctacagtccactttaagttgtcccctacatatgtattatatatggcatgccggtatgtattgtccaaccagtacagacctgacatcagccctacagagcgcacacataaagtcattgcagtcgtcaacaagatggtgagcatgatggagggtgtcatccagaaacagaagaatattgcaggggcacttgccttctggatggcaaatgcatctgaacttctcaacttcattaagcaagaccgagaccttagtcggatcacactggatgctcaagatgttttagcacatttggttcaaatggcatttaaatacttggttcactgtcttcaatcagaacttaataattacatgccagcctttctagatgaccctgaagagaacagtctgcaacgaccaaaaatagatgatgtgctgcacacgctcacaggagccatgtccttgctacgacgctgcagagtcaatgccgccctgaccatccagctcttctctcagctcttccacttcatcaatatgtggctgttcaatagattggtgaccgacccagattcggggctgtgctcccattactggggtgcgattatccgtcagcagttgggccatattgaagcctgggctgagaagcaggggctggaactggctgcggactgtcatctgagcaggatcgtgcaggcaacgactttgcttaccatggataagtatgcacctgatgacattccaaatataaacagcacctgctttaagttaaattcattacaacttcaagccttattacagaactatcactgtgcacctgatgagccttttatcccaacggatcttatagaaaatgtagtgactgtggctgaaaacactgccgatgagctggcccgcagtgatggaagggaagtgcagttggaggaggatcctgatctgcagctgccgtttcttttgccagaagatggttattcttgtgatgttgtcagaaacattccaaatggtttacaagaatttttagaccctctgtgccagagaggattttgcaggttaattcctcacacacgttcaccaggtacttggacaatatattttgaaggtgcagattatgaaagtcaccttctgcgtgagaacacagagctggctcagcctctgaggaaagaacctgaaataatcactgtgaccctaaaaaagcagaatggaatgggccttagcattgttgcagcaaagggtgctggtcaagataaactaggaatctacgtgaagtcggttgtgaaaggaggtgctgcagatgtggatggacgtctagctgcaggtgatcagctcctcagtgtggatggacgaagtctggttggactctctcaggaaagggcggcagaactcatgacaagaacaagctctgtggtgacactggaagtagcaaagcagggtgccatctaccacggtctggccacccttctcaatcagccatcccccatgatgcagagaatttcagatcgtcgtggctcaggtaaaccccgaccaaagagtgaaggctttgagctctataataattcaactcaaaatgggtctcctgagagtcctcagctgccttgggcagaatatagtgaaccaaagaaattgcctggtgatgacagactgatgaaaaatagagctgatcaccgttccagccccaacgtagcaaatcagcctcctagtcctggagggaaaagtgcatatgcctctggaacaacagcgaagataacatctgtctctactggaaacctctgcactgaggagcagacgcctccgcctagacctgaagcctaccccattcccactcagacgtacaccagagagtattttaccttcccagcttccaaatcccaggatcggatggctcctcctcagaaccagtggccaaattatgaggaaaagccacatatgcacacagatagtaatcattccagtattgcaattcagcgtgttacacgttcccaagaagaacttcgagaagataaagcttaccaacttgagcggcatcgaatagaggcagctatggaccgaaagtctgatagtgatatgtggataaatcagagctcctcactggactccagtacctctagccaggagcatctgaaccattcctctaagtcggtcacccctgcttccacactgaccaaaagtggccctggccgttggaaaacaccagcagccataccggccacccctgtggccgtctcccagccaatccgaacagacctgcctccgccacccccgccacctccagtccactatgccggtgatttcgatggaatgtccatggatttgcctctcccaccacccccttccgccaaccagatagggctgccgtctgcgcaggtggctgctgctgaacggagaaagagagaagaacatcagcgttggtatgagaaggagaaggcccccctggaggaggagcgggagaggaagcggagagagcaggagaggaagttgggccagatgcgcactcagtccttaaaccctgctccgttttctcccctgactgcacagcagatgaagcccgaaaagccttccacactccagcggccacaggaaacagtcattcgggagctgcagcctcagcagcagccccgcacgatcgagcgcagagacttgcagtacattacagtcagcaaagaggagctttcctcgggggacagtctgtcccccgacccgtggaagcgggacgccaaggagaagctggagaagcagcagcagatgcacatcgtggacatgctgagcaaggagatccaggagctccagagcaaaccggaccgcagcgccgaggagagcgaccggctgcgcaagctcatgctggagtggcagttccagaagagactccaggagtcgaagcagaaggacgaagatgacgaggaggaggaggacgatgatgtggacaccatgctgatcatgcagcgcctggaggctgaacgaagagcgagggtaaaggggggagtgctttggctgtgcccatctgtggtccctattttagcttctgcgtgtttcccatggggatag

SEQ ID NO: 90 - DNA, 3376 bp, Homo sapiens
tttggggctgagtttaataagcgagcgagcgagcaagcgagcgcggggggaaaaaggcagagaatgtccgccatctaccctccgctcctgggcgcgctctcattcatagcagcctcttcatgaattacagctgagggggggcggaggaggggggggtaccacacaacaccccagcaaacctccgggcccccaggcatggctagctcgtgttccgtgcaggtgaagctggagctggggcaccgcgcccaggtgaggaaaaaacccaccgtggagggcttcacccacgactggatggtgttcgtacgcggtccggagcacagtaacatacagcactttgtggagaaagtcgtcttccacttgcacgaaagctttcctaggccaaaaagagtgtgcaaagatccaccttacaaagtagaagaatctgggtatgctggtttcattttgccaattgaagtttattttaaaaacaaggaagaacctaggaaagtccgctttgattatgacttattcctgcatcttgaaggccatccaccagtgaatcacctccgctgtgaaaagctaactttcaacaaccccacagaggactttaggagaaagttgctgaaggcaggaggggaccctaataggagtattcataccagcagcagcagcagcagcagcagtagcagcagcagcagcagcagcagcagcagcagtagcagcagcagcagcagcagcagcagcagcagtagcagcagcagtagcagcagcagcagcagcagtagtaccagtttttcaaagcctcacaaattaatgaaggagcacaaggaaaaaccttctaaagactccagagaacataaaagtgccttcaaagaaccttccagggatcacaacaaatcttccaaagaatcctctaagaaacccaaagaaaataaaccactgaaagaagagaaaatagttcctaagatggccttcaaggaacctaaacccatgtcaaaagagccaaaaccagatagtaacttactcaccatcaccagtggacaagataagaaggctcctagtaaaaggccgcccatttcagattctgaagaactctcagccaaaaaaaggaaaaagagtagctcagaggctttatttaaaagtttttctagcgcaccaccactgatactcacttgttctgctgacaaaaaacagataaaagataaatctcatgtcaagatgggaaaggtcaaaattgaaagtgagacatcagagaagaagaaatcaacgttaccgccatttgatgatattgtggatcccaatgattcagatgtggaggagaatatatcctctaaatctgattctgaacaacccagtcctgccagctccagctccagctccagctccagcttcacaccatcccagaccaggcaacaaggtcctttgaggtctataatgaaagatctgcattctgatgacaatgaggaggaatcagatgaagtggaggataacgacaatgactctgaaatggagaggcctgtaaatagaggaggcagccgaagtcgcagagttagcttaagtgatggcagcgatagtgaaagcagttctgcttcttcacccctacatcacgaacctccaccacccttactaaaaaccaacaacaaccagattcttgaagtgaaaagtccaataaagcaaagcaaatcagataagcaaataaagaatggtgaatgtgacaaggcatacctagatgaactggtagagcttcacagaaggttaatgacattgagagaaagacacattctgcagcagatcgtgaaccttatagaagaaactggacactttcatatcacaaacacaacatttgattttgatctttgctcgctggacaaaaccacagtccgtaaactacagagttacctggaaacatctggaacatcctgaggatataacaactggatgcatcaagaactattgtgttttttttttttggttttttttttttttggttgtgattttttgttcttgttgtttatatgaaaacactcaaaatgatgcaaccaaaagggaaaaaataaaaatcaaacaaccttcagctttatttttctttaaagccagtcatcatctcttgataaaggagaggttaaagcaaaccagcctcagcggaccactcttctctccaaggaaatccccgggaagagttagcctggatagccttgaaaacaaacaaatcaaacacaacacaagaaaactcaaagaatgtgtatggtatcatgtatctctctgtggtggttcattccacaggacgaatgcatattcaacacactgccttattacataactgatctatttattatcgcatacagatattctaagtcgttgagggaatgacaccatcagacattataagtacttggtcccgtggatgctctttcaatgcagcacccttgccatcccaagcccagtgaccttactcgtataccgtgccactttccaccaactttttccaagtcctttaactcgttgcagtctgtattttccaccttttgtttttccagttccaggacacagattatcaactggggggaccaaatagccaccttgattttcttctttgtggtctttttcctgaaagttggggcccagtccttggctgtatccatgtaatgatcttggaccatggtagaaaatgcaccaaataggatcatatgaattgctgtctagccttagtcaataaacttgtaggacttttaaacaaaagtgtacctgtaaatgtcctgaatccagcattgttgagctgtcatcaacattcttgtgtctgttttactgttacaatattaggtgaatatggaagtaaaggcattccacaggatcatcatttaaaaaaaaagaattctggtcctgttttctaaaaaaaaaaaactgttgtagaaattcttaatttggatctatttattagtcagagtttcagctttcttcagctgccagtgtgttactcatctttatcctaaaaatctggaatcagagatttttgtttgttcacatatgattctcttagacacttttatatttgaaaaaattaaaatctttctttggggaaaaattcttggttattctgccataacagattatgtattaacttgtagattcagtggttcaatacctgtttagttgcttgctaatatttccagaaggatttcttgtattggtgaaagacggttggggatggggggatttttttgttcttgttgtacccttgttttgaaactagaaatctgtcctgtggcatgcaaaagaaagcaaattatttttaaaagaaaaaaaccaaagtacttttggtgtcattattccatcttctccataagtggagaaatgaaaagtaagaacagctcatcttcaaagtttttactagaaattc

SEQ ID NO: 91 - DNA, 9390 bp, Homo sapiens
ggcaatttcttttcctttctaactgtggcccgcgttgtgctgttgctgggcaggcgttgggcgccggcggtcttcgagcgtgggggcccgctggctttcccttctcagaaactgcgccgggggcgctcgcttgccccggattcggacgcggcgctccccgggctcgtctgaagtgcagatcgccgcagaggccccagtgcccggatgtccatcaggattagcgcgagccaatacgggccgagcccggggctgcgccgaggacgcccggggctcgagagcaggtagtcccgtaacatcggggcgccgcgccgggacgcgtccccgcccggctccgccaaatggtgagcgcggcgctggcagcagggcccgcggggtgaaggcgctcatggacggaagacccctggctctataagctgaattatggcagcccagtcaagtttgtacaatgacgacagaaacctgcttcgaattagagagaaggaaagacgcaaccaggaagcccaccaagagaaagaggcatttcctgaaaagattcccctttttggagagccctacaagacagcaaaaggtgatgagctgtctagtcgaatacagaacatgttgggaaactacgaagaagtgaaggagttccttagtactaagtctcacactcatcgcctggatgcttctgaaaataggttgggaaagccgaaatatcctttaattcctgacaaagggagcagcattccatccagctccttccacactagtgtccaccaccagtccattcacactcctgcgtctggaccactttctgttggcaacattagccacaatccaaagatggcgcagccaagaactgaaccaatgccaagtctccatgccaaaagctgcggcccaccggacagccagcacctgacccaggatcgccttggtcaggaggggttcggctctagtcatcacaagaaaggtgaccgaagagctgacggagaccactgtgcttcggtgacagattcggctccagagagggagctttctcccttaatctctttgccttccccagttccccctttgtcacctatacattccaaccagcaaactcttccccggacgcaaggaagcagcaaggttcatggcagcagcaataacagtaaaggctattgcccagccaaatctcccaaggacctagcagtgaaagtccatgataaagagacccctcaagacagtttggtggcccctgcccagccgccttctcagacatttccacctccctccctcccctcaaaaagtgttgcaatgcagcagaagcccacggcttatgtccggcccatggatggtcaagatcaggcccctagtgaatcccctgaactgaaaccactgccggaggactatcgacagcagacctttgaaaaaacagacttgaaagtgcctgccaaagccaagctcaccaaactgaagatgccttctcagtcagttgagcagacctactccaatgaagtccattgtgttgaagagattctgaaggaaatgacccattcatggccgcctcctttgacagcaatacatacgcctagtacagctgagccatccaagtttcctttccctacaaaggactctcagcatgtcagttctgtaacccaaaaccaaaaacaatatgatacatcttcaaaaactcactcaaattctcagcaaggaacgtcatccatgctcgaagacgaccttcagctcagtgacagtgaggacagtgacagtgaacaaaccccagagaagcctccctcctcatctgcacctccaagtgctccacagtcccttccagaaccagtggcatcagcacattccagcagtgcagagtcagaaagcaccagtgactcagacagttcctcagactcagagagcgagagcagttcaagtgacagcgaagaaaatgagcccctagaaaccccagctccggagcctgagcctccaacaacaaacaaatggcagctggacaactggctgaccaaagtcagccagccagctgcgccaccagagggccccaggagcacagagcccccacggcggcacccagagagtaagggcagcagcgacagtgccacgagtcaggagcattctgaatccaaagatcctccccctaaaagctccagcaaagccccccgggccccacccgaagccccccaccccggaaagaggagctgtcagaagtctccggcacagcaggagcccccacaaaggcaaaccgttggaaccaaacaacccaaaaaacctgtcaaggcctctgcccgggcaggttcacggaccagcctgcagggggaaagggagccagggcttcttccctatggctcccgagaccagacttccaaagacaagcccaaggtgaagacgaaaggacggccccgggccgcagcaagcaacgaacccaagccagcagtgcccccctccagtgagaagaagaagcacaagagctccctccctgccccctctaaggctctctcaggcccagaacccgcgaaggacaatgtggaggacaggacccctgagcactttgctcttgttcccctgactgagagccagggcccaccccacagtggcagcggcagcaggactagtggctgccgccaagccgtggtggtccaggaggacagccgcaaagacagactcccattgcctttgagagacaccaagctgctctcaccgctcagggacactcctcccccacaaagcttgatggtgaagatcaccctagacctgctctctcggataccccagcctcccgggaaggggagccgccagaggaaagcagaagataaacagccgcccgcagggaagaagcacagctctgagaagaggagctcagacagctcaagcaagttggccaaaaagagaaagggtgaagcagaaagagactgtgataacaagaaaatcagactggagaaggaaatcaaatcacagtcatcttcatcttcatcctcccacaaagaatcttctaaaacaaagccctccaggccctcctcacagtcctcaaagaaggaaatgctccccccgccacccgtgtcctcgtcctcccagaagccagccaagcctgcacttaagaggtcaaggcgggaagcagacacctgtggccaggaccctcccaaaagtgccagcagtaccaagagcaaccacaaagactcttccattcccaagcagagaagagtagaggggaagggctccagaagctcctcggagcacaagggttcttccggagatactgcaaatccttttccagtgccttctttgccaaatggtaactctaaaccagggaagcctcaagtgaagtttgacaaacaacaagcagaccttcacatgagggaggcaaaaaagatgaagcagaaagcagagttaatgacggacagggttggaaaggcttttaagtacctggaagccgtcttgtccttcattgagtgcggaattgccacagagtctgaaagccagtcatccaagtcagcttactctgtctactcagaaactgtagatctcattaaattcataatgtcattaaaatccttctcagatgccacagcgccaacacaagagaaaatatttgctgttttatgcatgcgttgccagtccattttgaacatggcgatgtttcgttgtaaaaaagacatagcaataaagtattctcgtactcttaataaacacttcgagagttcttccaaagtcgcccaggcaccttctccatgcattgcaagcacaggcacaccatcccctctttccccaatgccttctcctgccagctccgtagggtcccagtcaagtgctggcagtgtggggagcagtggggtggctgccactatcagcaccccagtcaccatccagaatatgacatcttcctatgtcaccatcacatcccatgttcttaccgcctttgacctttgggaacaggccgaggccctcacgaggaagaataaagaattctttgctcggctcagcacaaatgtgtgcaccttggccctcaacagcagtttggtggacctggtgcactatacacgacagggttttcagcagctacaagaattaaccaaaacaccttaatggagccccaggttgattcaatgccttgggaactatttttgcacattggaagcctcaaaaacagtccagacgtttgtttcatcaggacaccaaactctaaaaaagaagcaccacgagatggccaggacatttgtccacttaaactctcaacaacagtgtgatcattggttggacactgtggttatgcagaagcagagatgaggaggctggccccagagatgatcttgcccttcctaactaaaggacagaagtgcaatttagcttaaatgggtgtatgaatggtctagaaacatttctattttttttttaaaccagcaggatacaagttgcaaatgaaatgaggagaaacagtttcaactctgaaagtgaatttcacgtcatctcagtagccacgctagtccattcccagaaggaaatttttttttttaacaatgacttttggtaaagggttttgtggatgattttttttcttttgagttttgggagaaatatttgtttaataacttctaatggccatctgtaaaccataagtaatgaaggactccactgtgccccactttctgccaatgaacagtggcttgataataccaagtattgttgtaatttataaaattgaaggcaacccccgctcctgccgcccccaatctccccattgcctagagcgctgcacattgaccccagctctgacttctcattactgtgctgaaagtcagcccacgtcggagcggtgaggaggagccacagcacatggggtgccacctcgaggtctgcacaggaggacttggcgctgccatttcctacccctgccatttcccacccctgcttcagcgaaagggactctctaacagggcagtcactgttgactctattctgaatttcctcccttggggaagaagggaaccaacatttatacctgaccagatggctaaagtgcttttaaagttttgtttaagtagagctggaatttgaggtgctgatctgtggtctacagttatgtggtaactcatgttgtccagccaactcagagtttcgtcagtgaacaagaaacatgaaatctgcttcttagagaggctatatttttctgctacaaatattttatatttatagcaaaactagactttcagagtccttgattgtctaggggaagttaactccctgagaggatgtagagatttggggtggttgattagacttttgaaaaactcatcaccacatgccttcactccagagtgttctcagctagatttgatttggttgaggaggaactgtggccctccgtaagttattgccatagtgtatgcattaaaccaagtccattttgaatgacctaaaatgaagtaacacaatcagaaatcccatgtgcccataagcacagatttttctttttcattgaaactttaaaggttattattggaaacattactttgagtgcagtgtttttaaaagccaattcttttttatcccttttagaagtagaatttgcacacttactacaattgaggagtgtcatctctataactttttctccgcctttgtcccattctgcccctggacatgtttcctaccaagcatgtttcacattttcctattagtggaggagggagaaccatatttatttataatgaagacatctaagatccctatgatgaatgcaggaactctcttggtagtttgtaaatacacaaagggatgtgtcgagggatgggagcgatgcttatctctcacagtgtgagtggtctgtgtgaggctgttccttcagttcttctccagactgttctttggttgtcacttaagtcagaggtctggtccctcatgtttaggtgaaagccagagaatgacagctgtagtcatatctgagcataagaccttgatgtgtgattcctgatgaccggtttcatttattcatgtaataaagcaaaggccctggtcctttttaaactactagttttaaaaacctgtgttaaatgaacagtaattgcctggtaggtttggtgtgtgtgtagcattgtgtgtccatctgttatatgtaaaggacaaggcaccagaatcaggctttatttcgatattgaagatgttatttaacatctttcttttttccttactcccttagccatcccctccccttttgtcctatcattccctagaacaagccacctgtcaattgtgaagggttgtgttctttatggcaggttctatgcagattgtgccagagcatgtgcgtgttctgttggcaagccacagtgctcccttgactgaagacatttccaggtagatttctcagccagctctaaaacagattgctttttcagtggccttactctttgtgggtttttttttttctctgaacttgatataaagattttatttgtcccttgaaaaagtaacaaatgtgcatagatcaatttgtactactttggtcattggatatttctgatccttattgcattgtacctaaaggagagtaactaatggtaacctttttaatagagtatgtgaaaggtagtggctgatgaatccttaacgttcatagggtctttttgctgttacggttgtatatagaggtctgaaggatttttaaaatgatttgcactttttcactgcatgcttacaattcccaaaggcaaaatctgtactgaggtagatcatttgaaagggctagattataaaattaagccttagagtatggaaagttcttataacaataatagtacacacttcagagtaagacaaatgcaaagcatcttaaggagtgaaaatagagtctaaatcttgcctttggcactacaaggtgtgtgtgtgtgtgtgtgttgtgtgtctttagtaggaaatggaagaacactgttttattttttaaagtgtttaatgtttctgtcctttctgtgaattattgaatttaagagccctgctaaataatgaaaaaacactttactaaaatttatcaaattatactgggttcggattgtgaaaacattggccacctagtagcagtggtgaggagtgggagggcccagcaagcatttatcagaaatagaatcacaataggaggagaatttggctgtctgatattatgatttgattacaatactgaatgggaaaagtatctaatattttgtaacaaaaagaccttcatattatctgttttgaccaaaatatgtagctatttcccttacacagattggaccgcacttatctcccttgtcctgtatcctttaatttcaggtctcaggatgtttagaaagctaaaaccccctacccctttctggctgaaaacttgccttatttggtatcttacacattaatgttactagcatcaggagcttactgttttattatgattcatcttcagtaatttttagaagcaagaagaaagccattgtgtcctctacaattaacaaaacttatctctgatatacaaagggatataaatatatacacttaaatagagaaaaagaggttgattgaattgtgcctttgagtgaacccagtttttaaataccgctgtgtttgtttcgccatggcttcagggatgctacatggctcttgcaccttttactcctctgctttatgaagtttgagttgtatttgtgcatcttaaagtaggttgaggcttgaggctgggctttcgggtttttttgttttttgttttgttttgttttgttttgttttcttgtacttaaacctgcttgcttcctaccacagattctttattttcccaaacactacaaaaaaacttttaaaactttgccatttcatctgtttacactctttgccactgattagcagtatttaaatcttgcaagaatattttgtgctttctttagaaacacaagagtatagatttttctcactgaaaagtgagagttacgcattgcagccatgaagggatgctaggatcaattatggcagtaccttttttcccctcctgttcttgagccagttgtctcttttgttttgggtcccacttaggattaacggatgtaaggtattttcctgtgcctttattttgtgtcattctattggaaggaggtgtaacggcagaatagcatcgtgttgggggttttccttcaaacactgcaagtgatattgccaccatgtgaacctcaaatatgcaatccagttgtgttggtttctcggtgacttggagtgttcatctcttcatgaattgtgagcactgaccatgttcttcagttcttaattatggtgagttgacaaataccaactactgcttttctttaggtggctataaatttcttactgtcaggaggaaatgacattatattctgttccactgaacgtcagagatcagcaggcactgtactgggtagagaagtgcctatacttctctacctaagagggcaggagggaaaccctacagctccttgtgagcctatatattagtatatcggcctggagaggacaagggaataagaccactcatagtgaggctggccaagctgcactggtcggaccaggcagtggctgacctaaggaaggcaacttgctttgcttaaaagtagattttttaagcaatgcttaacacaggcagcattcacctttgttcaggccatcgacatgtattgttaaaattactgcatatccccctcagatatcaagtatacactgttcatgttggggttgtgtgtgtgtatgtgtgtatgtacgcacgcatgtgtcccaaatcttgttttaattttttttttctgaatgtgatcatgttttggataatacctgagcagggttgccttttttttatttattaccattatatattatattatattatatattttttgctttcttataactttggaggaaagtcaaatcttggtattattaaaattgttttaaaaaggagtaaattttccagttgataaatgaaaatcactggcctatgtttaataagtttttctttaattactgtggaataacgtgccagctatcatcaacacaatgattttgtacatagggtagggaagcagtgatgctctcaatgggaagatgtgcaacacaaattaaggggaactccatgtattttacctacttcagcaatggaactgcaacttggggctttgtgaataaaatttagctgccttgtatagtcgtttgaaagaatatgtgatctgtgagagaattatagtttttttttagaagaaaaatctgcaaaagatctttccaaagacaatgtgccacagatcttttgttctctgtaatgaggattaattgctgtttaaacaaaaatgtaattgttcatctttaaattctttccttttcataagaggatcaagctgtaaaaaaacaaaaaaattaataaaaatttcgagaaatcaaaaaaaaaaa

SEQ ID NO: 92 - DNA, 3842 bp, Homo sapiens
gccctcttgattatgtgtgccctctccgggcgcccgcgttagcggccgggtggaggtggggagggaagacgctgaggaggaggaggaggcggaggaggcggtggaggggaggtggggggaatcagcaaggacatggctcctgactcctgtgcggaacgtgagtgactgagcggcaaagcccgaatggtctctagcgaccggcccgtgtcactggaggacgaggtctcccatagtatgaaggagatgattggaggctgttgcgtttgctcagacgagagaggctgggccgagaacccgctggtttattgcgacgggcacggctgcagcgtcgcggtgcatcaagcttgctatggcattgttcaagtacccactggaccgtggttttgcaggaaatgtgaatctcaggagagagcagccagagtgagatgtgaactttgtccccataaggatggagctttaaaaagaacagataatgggggttgggcccatgtggtttgtgccctgtatattccagaggtacaatttgccaatgtttccacaatggaaccaattgttttacagtctgttccgcatgatcgttataataagacttgctacatttgtgatgaacaaggaagagaaagcaaagcagccactggtgcttgcatgacatgtaataaacatggatgtcgacaggctttccatgtaacatgcgctcagtttgccggactgctttgtgaagaagaaggtaatggtgccgataatgtccaatactgtggctactgtaaataccattttagtaagctgaaaaagagcaaacggggatctaataggtcatatgatcaaagtttaagtgattcttcctctcactctcaggataaacatcatgagaaagagaaaaaaaaatataaagagaaggacaaacacaaacagaaacacaagaagcagccagaaccatcacctgcattggttccatccttgactgttactacagaaaaaacttatacaagcactagcaacaactctatatctggatcattgaagcgcttggaagatactactgcacgatttacaaatgcaaatttccaggaagtctctgcacacacctctagtggaaaagatgtttcagagactagagggtcagagggcaaagggaagaaatcttcagctcacagctcaggtcaaaggggaagaaagcctggtggtggaagaaatccaggaacaactgtgtcagcagctagcccttttcctcaaggcagtttttcaggaactccaggcagtgtaaagtcatcttctggaagttcagtgcagtctccccaggatttcctgagctttacagactcagatctgcgtaatgacagttactctcactcccaacagtcatcagcaaccaaagatgtacataaaggagagtctggaagccaggaagggggggtaaatagttttagtaccttaattggcctcccttcaacctcagctgttacttcacagcctaaaagctttgaaaattcacctggagatttgggtaattccagccttcctacagcaggatataagcgggctcaaacttctggcatagaagaagaaactgtaaaggaaaagaaaaggaaaggaaataaacaaagtaagcatgggcctggcagacccaaaggaaacaaaaatcaagagaatgtttctcatctctcagtttcttctgcttcaccaacatcatctgtagcatcagctgcaggaagcataacaagctctagtctgcagaaatctcctacattgctcaggaatggaagtttacagagcctcagtgttggctcatctccagttggttcagaaatttccatgcagtatcggcatgatggagcttgcccaacaactacgttctcagagttgctgaatgcaatacacaacgacagaggtgacagttctacactaacaaagcaagaacttaaattcataggtatttataacagcaatgatgtagcagtatcgtttccaaatgtagtatctggctcgggatctagtactcctgtctccagctctcacttacctcagcagtcttctgggcatttgcaacaagtaggagcgctctctccctcagctgtgtcatctgcagcccctgctgttgctacaactcaggcaaatactctatctggatcttctctcagtcaggcaccatctcatatgtatggcaatagatcaaattcatcaatggcagctcttatagctcagtctgaaaacaatcaaacagatcaagatcttggagacaatagccgcaacctagttggcagaggaagctcaccccgaggaagtctctcgccacgatcccctgtaagcagcttacagattcgctatgatcaaccaggcaacagcagtttggaaaatctgcctccagtagcagccagcatagaacagcttttggagaggcagtggagtgaaggacagcaatttttactagaacagggtactcctagtgacattttaggaatgctgaagtcattacaccaacttcaagttgaaaaccgaagattagaggaacaaattaaaaacttgactgccaaaaaggaacggcttcagttattgaatgcacagctttcagtgccttttccaacaataacagcaaatcctagtccgtctcatcaaatacacacattttcagcacagactgctcctactactgattccttgaacagcagtaagagccctcatataggaaacagctttttacctgataattctcttcctgtattaaatcaggacttaacctccagtggacaaagtaccagcagctcatcagctctttctaccccacctcctgctgggcagagtccggctcaacaaggctcaggagtgagtggagttcagcaggtcaatggcgtgacagtgggggcactagctagtggaatgcagcctgtaacttccaccattcctgccgtgtctgcagtgggtggaataattggagctttgccaggtaaccaactggcaattaatggcattgtaggagctttaaatggggttatgcagactcctgtcacaatgtcccagaaccctacccctctcacccacacaaccgtaccacctaatgcaacacatccaatgccagctacactgactaacagtgcctcaggactaggattactttctgaccagcaacgacaaatacttattcatcaacagcagtttcagcagttgttaaattctcaacagctcacaccagtacacaggcacccccacttcacacagctaccaccaacccatttctcaccatccatggagataatgcaagtcagaaagtagcaagacttagtgataaaactgggcctgtagctcaagagaaaagttgacacctgagaaacatctagaaattgcctatcctgctgttctagcacttcatctggctgcctttgcagtccttttactacagctatgaagaaacgcaacaagaaactcaatgcacaacaaaggattaattgctgcaaggacattcttgtaaggctttgattagttttcttgttgctttgttgcactgaaatggaattcccatgcccctaccccttaccccagttttttgaacatggaaagaaaatttaataactttttaaagtgacataatttacatgcaatatgtttatcaactcaagaatttaatatagttggtacacaactagttttgtttataaattggagatgcaaatagcaaaactaaatacttgctccatttacaaactacttgattttattgtacaagttgaaatatgctcttttgtttgggttacagtatgcttgctctaagtcaaattccaaggaactaatttcttctcctggagttgcattgattcagtattacaaatatatagcacatcacctgggac

SEQ ID NO: 93 - DNA, 15750 bp, Homo sapiens
atggacagccagaacctggctggtgaggataaagattcacaaccggcagctgatggacctgcagcctctgaggacccaagtgccactgagtcagacctgcccaacccacatgtgggagaggtctctgtccttagttctgggagtcccaggcttcaggagactcctcaggactgcagtgggggtccggtgcggcgttgtgctctctgtaactgcggggagcccgctctacacgggcagcgggagctacggcgctttgagttgccatttgattggccccggtgtccagtggtgtcccctggggggagcccagggcccaatgaggcagtgctgcccagtgaggacctatcacagattggtttccctgagggccttacacctgcccacctaggagaacctggagggtcctgctgggctcaccattggtgtgctgcatggtcggcaggcgtatgggggcaggagggcccacaactatgtggtgtggacaaggccatcttctcagggatctcacagcgctgctcccactgcaccaggctcggtgcctccatcccttgccgctcacctggatgtccacggctttaccacttcccctgcgcgactgccagcggttccttcctatccatgaaaacactgcagctgctatgcccagagcacagtgagggggctgcatatctggaggaggctcgctgtgcagtgtgtgaggggccaggggagctgtgtgacctgttcttctgtaccagctgtgggcatcactatcacggggcctgcctggacactgctctgactgcccgcaaacgtgctggctggcagtgccctgaatgcaaagtgtgccaagcctgcaggaaacctgggaatgactctaagatgttggtttgcgagacgtgtgacaaaggataccatactttctgcctaaaaccacccatggaggaactgcctgctcactcttggaagtgcaaggcgtgccgggtgtgccgggcctgtggggcgggctcagcagaactgaatcccaactcggagtggtttgagaactactctctctgtcaccgctgtcacaaagcccagggaggtcagactatccgctccgttgctgagcagcataccccggtgtgtagcagattttcacccccagagcctggcgatacccccactgacgagcccgatgctctgtacgttgcatgccaagggcagccaaagggtgggcacgtgacctctatgcaacccaaggaaccagggcccctgcaatgtgaagccaaaccactagggaaagcaggggtccaacttgagccccagttggaggcccccctaaacgaggagatgccactgctgcccccacctgaggagtcacccctgtccccaccacctgaggaatcacccacgtccccaccacctgaggcatcacgcctgtcgccaccacctgaggaattgcccgcatccccacttcctgaggcattgcacctgtcccggccgctggaggaatcgcccctctctccgccgcctgaggagtctcctctgtctcccccacctgaatcatcacctttttctccactggaggagtcgcccttgtctccaccggaagagtcacccccatctcctgcacttgagacgcctctatccccaccacctgaagcatcgcccctgtccccaccatttgaagaatctcctttgtccccgccacctgaggaattgcccacttccccgccacctgaagcatctcgcctgtctccaccacctgaggagtcacccatgtcccctccacctgaagagtcacccatgtctccaccaccggaggcatctcgtctgttcccaccatttgaagagtctcctctgtcccctccacctgaggagtctcccctttccccaccacctgaggcatcacgcctgtccccaccacctgaggactcgcctatgtccccaccacctgaagaatcacctatgtcccccccacctgaggtatcgcgcctatcccccctgcctgtggtgtcacgcctgtctccaccgcctgaggaatctcccttgtccccaccggccctgtctcctttgggggagttagagtacccctttggtgccaaaggggacagtgaccctgagtcaccgttggctgcccccatcctggagacacccatcagccctccaccagaagctaactgcactgaccctgagcctgtcccccctatgatccttcccccatctccaggctccccagtggggccggcttctcccatcctgatggagccccttcctcctcagtgttcgccactccttcagcattccctggttccccaaaactcccctccttcccagtgctctcctcctgccctaccactgtccgttccctccccgttgagtcccatagggaaggtagtgggggtctcagatgaggctgagctgcacgagatggagactgagaaagtttcagaacctgaatgcccagccttggaacccagtgccaccagtcctctcccttccccaatgggggacctttcctgccccgcccccagccctgccccagccctggatgacttctctggcctaggggaagacacagcccctctggatgggattgatgctccgggttcacagccagagcctggacagacccctggcagtttggctagtgaacttaaaggctcccctgtgctcctggaccccgaggagctggcccctgtgacccctatggaggtctaccccgaatgcaagcagacagcagggcggggctcaccatgtgaagaacaggaagagccacgtgcaccggtggcccccacaccacccactctcatcaaatccgacatcgttaacgagatctctaatctgagccagggtgatgccagtgccagttttcctggctcagagcccctcctgggctctccagacccggaggggggtggctccctgtccatggagttgggggtctctacggatgttagtccagcccgagatgagggctccctacggctctgtactgactcactgccagagactgatgactcactattgtgcgatgctgggacagctatcagcggaggcaaagctgagggggagaaggggcggcggcgcagctccccagcccgttcccgcatcaaacagggtcgcagcagcagtttcccaggaagacgccggcctcgtggaggagcccatggagggcgtggtagaggacgggcccggctaaagtcaactgcttcttccattgagactctggtagttgctgacattgatagctctcccagtaaggaggaggaggaagaagatgatgacaccatgcagaataccgtggttctcttctccaacacagacaaatttgtcctaatgcaggacatgtgtgtggtatgtggcagctttggccggggggcagagggccacctccttgcctgttcgcagtgctctcagtgctatcacccttactgtgtcaacagcaagatcaccaaggtgatgctgctcaagggctggcgttgtgtggagtgtattgtgtgtgaggtgtgtggccaggcctccgacccctcacgcctgctgctctgtgatgactgtgatattagctaccacacatactgcctggaccccccactgctcaccgtccccaagggcggctggaagtgcaagtggtgtgtgtcctgtatgcagtgtggggctgcttcccctggcttccactgtgaatggcagaatagttacacacactgtgggccctgtgccagcctggtgacctgccctatctgtcatgctccttacgtagaagaggacctactaatccagtgccgccactgtgaacggtggatgcatgcaggctgtgagagcctcttcacagaggacgatgtggaccacgcacccgatgaaggctttgactgtgtctcctgccagccctacgtggtaaagcctgtggcgcctgttgcacctccagagctggtgcccatgaaggtgaaagagccagagccccagtactttcgcttcgaaggcgtgtggctgacagaaactggcatggccttgctgcgtaacctgaccatgtcaccactgcacaagcggcgccaacggcgaggacggcttggcctcccaggcgaggcaggattggagggttctgagccctcagatgcccttggccctgatgacaagaaggatggggacctggacaccgatgagctgctcaagggtgaaggtggtgtggagcacatggagtgcgaaattaaactggagggccccgtcagccctgatgtggagcctggcaaagaggagaccgaggaaagcaaaaaacgcaagcgtaaaccatatcggcctggcattggtggtttcatggtgcgacagcggaaatcccacacacgcacgaaaaaggggcctgctgcacaggcggaggtgttgagtggggatgggcagcccgacgaggtgatacctgctgacctgcctgcagagggcgccgtggagcagagcttagctgaaggggatgagaagaagaagcaacagcggcgagggcgcaagaggagcaaactggagggcatgttccctgcttacttgcaggaagccttctttgggaaggagctgctggacctgagccgtaaggccctttttgcagttggggtgggccggccaagctttggactagggaccccaaaagccaagggagatggaggctcagaaaggaaggaactccccacatcgcagaaaggagatgatggtccagatattgcagatgaagaatcccgtggcctcgagggcaaagccgatacaccaggacctgaggatgggggcgtgaaggcatccccagtgcccagtgaccctgagaagccaggcaccccaggtgaagggatgcttagctctgacttagacaggatttccacagaagaactgcccaagatggaatccaaggacctgcagcagctcttcaaggatgttctgggctctgaacgagaacagcatctgggttgtggaacccctggcctagaaggcagccgtacgccactgcagaggccctttcttcaaggtggactccctttgggcaatctgccctccagcagcccaatggactcctacccaggcctctgccagtccccgttcctggattctagggagcgcgggggcttctttagcccggaacccggtgagcccgacagcccctggacgggctcaggtggcaccacgccctccacccccacaacccccaccacggagggtgagggcgacggactctcctataaccagcggagtcttcagcgctgggagaaggatgaggagttgggccagctgtccaccatctcgcctgtgctctatgccaacattaattttcctaatctcaagcaagactacccagactggtcaagccgttgcaaacaaatcatgaagctctggagaaaggttccagcagctgacaaagccccctacctgcaaaaggccaaagataaccgggcagctcaccgcatcaacaaggtgcagaagcaggctgagagccagatcaacaagcagaccaaggtgggcgacatagcccgtaagactgaccgaccggccctacatctccgcattcccccgcagccaggggcactgggcagcccgccccccgctgctgcccccaccattttcattggcagccccactacccccgccggcttgtctacctctgcggacgggttcctgaagccgccggcgggctcggtgcctggccctgactcgcctggtgagctcttcctcaagctcccaccccaggtgcccgcccaagcgccttcgcaggacccctttggactggcccctgcctatcccctggagccccgcttccccacggcaccgcccacctatcccccctatcctagtcctacgggggcccctgcgcagcccccgatgctgggcgcctcatctcgtcctggggctggccagccaggggaattccacactaccccacctggcacccccagacaccagccctccacacctgacccgttcctcaaaccccgctgcccctcgctggataacttggctgtgcctgagagccctggggtagggggaggcaaagcttccgagcccctgctctcgcccccaccttttggggagtcccggaaggccctagaggtgaagaaggaagagcttggggcatcctctcctagctatgggcccccaaacctgggctttgttgactcaccctcctcaggcacccacctgggtggcctggagttaaagacacctgatgtcttcaaagcccccctgacccctcgggcatctcaggtagagccccagagcccgggcttgggcctaaggccccaggagccaccccctgcccaggctttggcaccttctcctccaagtcacccagacatctttcgccctggctcctacactgacccatatgctcagcccccattgactcctcggccccaacctccgccccctgagagctgctgtgctctgccccctcgctcactgccctccgaccctttctcccgagtgcctgtcagtcctcagtcccagtccagctcccagtctccactgacaccccggcctctgtctgctgaagctttttgcccatcacccgttacccctcgcttccagtcccctgacccttattctcgcccaccctcacgccctcagtcccgtgacccatttgccccattgcataagccaccccgaccccagccccctgaagttgcctttaaggctgggtctctagcccacacttcgctgggggctggggggttcccagcagccctgcccgcggggccagcaggtgagctccatgccaaggtcccaagtgggcagccccccaattttgtccggtcccctgggacgggtgcatttgtgggcaccccctctcccatgcgtttcactttccctcaggcagtaggggagccttccctaaagccccctgtccctcagcctggtctcccgccaccccatgggatcaacagccattttgggcccggccccaccttgggcaagcctcaaagcacaaactacacagtagccacagggaacttccacccatcgggcagccccctggggcccagcagcgggtccacaggggagagctatgggctgtccccactacgccctccgtcggttctgccaccacctgcacccgacggatccctcccctacctgtcccatggagcctcacagcgatcaggcatcacctctcctgtcgaaaagcgagaagacccagggactggaatgggtagctctttggcgacagctgaactcccaggtacccaggacccaggcatgtccggccttagccaaacagagctggagaagcaacggcagcgccagcggctacgagagctgctgattcggcagcagatccagcgcaacaccctgcggcaggagaaggaaacagctgcagcagctgcgggagcagtggggcctccaggcagctggggtgctgagcccagcagccctgcctttgagcagctgagtcgaggccagaccccctttgctgggacacaggacaagagcagccttgtggggttgcccccaagcaagctgagtggccccatcctggggccagggtccttccctagcgatgaccgactctcccggccacctccaccagccacgccttcctctatggatgtgaacagccggcaactggtaggaggctcccaagctttctatcagcgagcaccctatcctgggtccctgcccttacagcagcaacagcaacaactgtggcagcaacaacaggcaacagcagcaacctccatgcgatttgccatgtcagctcgctttccatcaactcctggacctgaacttggccgccaagccctaggttccccgttggcgggaatttccacccgtctgccaggccctggtgagccagtgcctggtccagctggtcctgcccagttcattgagctgcggcacaatgtacagaaaggactgggacctgggggcactccgtttcctggtcagggcccacctcagagaccccgtttttaccctgtaagtgaggacccccaccgactggctcctgaagggcttcggggcctggcggtatcaggtcttcccccacagaaaccctcagccccaccggcccctgaattgaacaacagtcttcatccaacaccccacaccaagggtcctaccctgccaactggtttggagctggtcaaccggcccccgtcgagcactgagcttggccgccccaatcctctggccctggaagctgggaagttgccctgtgaggatcccgagctggatgacgattttgatgcccacaaggccctagaggatgatgaagagcttgctcacctgggtctgggtgtggatgtggccaagggtgatgatgaacttggcaccttagaaaacctggagaccaatgacccccacttggatgacctgctcaatggagacgagtttgacctgctggcatatactgatcctgagctggacactggggacaagaaggatatcttcaatgagcacctgaggctggtagaatcggctaatgaggaggctgaacgggaggccctgctgcggggggtggagccaggacccttgggccctgaggagcgccctccccctgctgctgatgcctctgaaccccgcctggcatctgtgctccctgaggtgaagcccaaggtggaggagggtggacgccacccttctccttgccaattcaccattgctacccccaaggtagagcccgcacctgctgccaattcccttggcctggggctaaagccaggacagagcatgatgggcagccgggatacccggatgggcacagggccattttctagcagtgggcacacagctgagaaggcctcctttggggccacgggagggccaccagctcacctgctgacccccagcccactgagtggcccaggaggatcctccctgctggaaaagtttgagctcgagagtggggctttgaccttgcctggtggacctgcagcatctggggatgagctagacaagatggagagctcactggtagccagcgagttacccctgctcattgaggacctgttggagcatgagaagaaggagctgcagaagaagcagcagctttcagcacagttgcagcctgcccagcagcagcagcaacagcagcagcagcattccctactgcctgcaccaggccctgcccaggccatgtctttgccacatgagggctcttctcccagtttggctgggtcccaacagcagctttccctgggtcttgcagttgcccgacagccaggtttgccccagccactgatgcccacccagccaccagctcatgccctccagcaacgcctggctccatccatggctatggtgtccaatcaagggcatatgctaagtgggcagcatggagggcaggcaggcttggtaccccagcagagctcacagccagtgctatcacagaagcccatgggcaccatgccaccttccatgtgcatgaagccgcagcaattggcaatgcagcagcagctggcaaacagcttcttcccagatacagacctggacaaatttgctgcagaagatatcattggtcccattgcaaaggccaagatggtggctttgaaaggcatcaagaaagtgatggctcagggcagcattggggtggcacctggtatgaacagacagcaagtgtctctgctagcccagaggctctcggggggacctagcagtgatctgcagaaccatgtggcagctgggagtggccaggagcggagtgctggtgatccctcccagcctcgtcccaacccgcccacttttgctcagggagtgatcaatgaagctgaccagcggcagtatgaggagtggctgttccatacccagcagctcctacagatgcagctgaaggtgctagaggagcagattggtgtacaccgcaagtcccggaaggctctgtgtgccaagcagcgcactgccaaaaaagctggccgtgagttcccagaagctgatgctgagaagctcaagctggttacagagcagcagagcaagatccagaaacaactggatcaggtccggaaacagcagaaggagcacactaatctcatggcagaatatcggaacaagcagcagcaacaacagcagcagcagcagcaacaacagcaacagcactcagctgtgctggctctcagcccttcccagagtccccggctgctcaccaagctccctggtcagctgctccctggccatgggctgcagccaccacaggggcctccgggtgggcaagccggaggtcttcgcctgacccctgggggtatggcactacctggacagcctggtggccccttccttaatacagctctggcccaacagcagcaacagcaacattctggtggggctggatccctggctggcccttcagggggcttcttccctggcaaccttgctcttcgaagcctcggacctgattcaaggcttttacaggaaaggcagctgcagctgcagcagcaacgtatgcagctggcccagaaactgcagcagcagcagcagcagcaacagcagcagcagcaccttctaggacaggtggcaatccagcagcaacagcagcagggtcctggagtacagacaaaccaagctctgggtcccaagccccagggccttatgcctcccagcagccaccaaggcctcctggtccagcagctgtcccctcaaccaccccaggggccccagggcatgctgggccctgcccaggtggctgtgttgcagcagcagcaccctggagctttgggcccccagggccctcacagacaggtgcttatgacccagtcccgggtgctcagttccccccagctggcacagcagggtcagggccttatgggacacaggctggtcacagcccagcagcagcagcagcaacaacagcaccaacagcaagggtccatggcagggctgtcccatcttcagcaaagtctgatgtcacacagtgggcagcccaaactgagcgctcagcccatgggctctttacagcagcttcagcagcagcagcagctgcaacagcaacagcaacttcagcagcagcagcagcagcagctacaacagcaacagcaacttcagcagcaacagcttcaacagcagcaacagcagcagcagcttcaacaacagcagcagcaacagcttcaacagcagcaacagcagctacaacagcaacagcaacaacaacagcagcagtttcaacagcagcagcaacagcagcagatgggccttttaaaccagagtcgaactttactgtcccctcagcaacaacagcagcagcaagtggcacttggccctggcatgccagcaaagcctcttcaacacttttctagccctggagccctgggtccaaccctcctcctgacgggcaaggaacaaaacaccgtagacccagccgtttcttcagaggccactgaggggccctctacacatcagggagggccgttagcaataggaactacccctgagtcaatggccactgaaccaggagaggtaaagccctcactctctggggactcacaactcctgcttgtccaaccccagccccagcctcagcccagctctctgcagctgcagccacctctgaggcttccaggacaacagcagcagcaagttagcctgctccacacagcaggtggaggaagccatgggcagctaggcagtggatcatcttctgaggcctcatctgtgccccacctgctggctcagccctctgtttccttaggggatcagcctgggtccatgacccagaaccttctgggcccccaacagcccatgctagagcggcccatgcaaaataatacagggccacaacctcccaaaccaggacctgtcctccagtctgggcagggtctgcctggggttggaatcatgcctacggtgggtcagcttcgagcacagctccaaggagtcctggccaaaaacccacagctgcggcacttaagtcctcagcagcagcagcagctacaggcactcctcatgcagcggcagctgcagcagagtcaggcagtacgccagaccccaccctaccaggagcctgggacccagacctctcccctccagggcctcctgggctgccaacctcaacttgggggcttccctggaccacagacaggccccctccaggagctaggggcagggcctcgacctcagggcccaccccggctccctgccccaccaggagccttatctacaggaccagtccttggccctgtccatcccacacctccaccatccagccctcaagagccaaagagaccttcacaattaccttcccccagctcccagcttcccactgaggcccagctccctcccacccatccagggacccccaaacctcaggggccaaccttggagccgcctcctgggagggtctcacctgctgctgcccagcttgcagataccttgtttagcaagggtctgggaccttgggatcccccagacaacctagcagaaacccagaagccagagcagagcagcctggtacctgggcatctggaccaggtgaatggacaggtggtgcctgaggcatcccaactcagcatcaagcaggaacctcgggaagagccatgtgccctgggagcccagtcagtgaagagggaggccaatggggagccaataggggcaccaggaaccagcaaccacctcctgctggcaggccctcgctcagaagctgggcatctgctcttgcagaagctactccgggcaaagaatgtgcaactcagcactgggcaggggtccgaggggctgcgagctgagatcaacgggcacattgacagcaagctggctgggctggagcagaaactacagggtacccccagcaacaaggaggatgcagcagcaaggaagcctttgacaccgaagcccaagcgggtacagaaggcaagcgacaggttggtgagctcccgaaagaagctgcggaaggaggacggcgtcagggccagcgaggccttgctgaaacagctgaaacaggagctgtccctgctgcccctaacggagcctgctatcaccgccaattttagcctctttgccccctttggcagtggctgcccagtcaatgggcagagccagctgaggggggcctttggaagtggggcgctgcccactggccctgactactattcccagctgcttaccaagaataacctgagtaacccgccgacaccaccctcgtcgctgccccccaccccacccccatcggtgcagcagaagatggtgaatggcgtcaccccatctgaagagctgggggagcaccccaaggatgctgcctctgcccgggatagtgaaagggcactgagggatacttcagaggtgaagagtctagacctgctggctgccttgcctacaccccctcacaatcagactgaggatgtcaggatggagagtgatgaggatagcgattctcctgacagcattgtgccagcttcatcccctgagagcatcttgggggaggaggcccctcgtttccctcatctgggctcaggccggtgggagcaagaggaccgggccctctcccctgtcatccccctcattcctcgggacagcatcccagtcttcccagataccaaaccttatggggcccttggcctggaggtccctggaaagctgcctgtcacaacttgggaaaagggcaaaggaagtgaggtgtcagtcatgctcacagtctctgctgctgcagacaagaacctgaatggcgtgatggtggcagtggcggagctgctgagcatgaagatccccaactcctatgaggtgctgttcccagagagccccgcccggggaggcactgagccaaagaagggggaagctgagggtcctggtgggaaggaaaagggtctggaaggcaagagcccagacactggccctgattggctgaagcagtttgatgcagtgttggctggctataccctgaagaggcaactagacatcttgagcctcctgaaacaggagagccccgccccagagccacccactcagcacaggtatacctacaatgtctccaatctggatgtgcgacagctctcggccccacctcctgaagaaccctccccgcccccttcccccttggcaccttctcctgccagtccccctactgagcccttggttgaacttcccaccgaacccttggctgagccacccgtcccctcacctctgccactggcctcatcccctgaatcagcccgacccaagccccgtgcccggccccctgaagaaggtgaagatacccgtcctcctcgcctcaagaaatggaaaggagtgcgctggaagcggcttcggctgctgctgaccatccagaagggcagtggacggcaggaggatgagcgggaagtggcagagtttatggagcagcttggcacagccttgcgacctgacaaggtaccgcgagacatgcgtcgctgctgtttctgtcatgaggagggtgacggggccactgatgggcctgcccgtctgctgaacctggacctggacctgtgggtgcacctcaactgtgccctttggtccacggaggtgtatgagacccagggcggagcactgatgaatgtggaggttgccctgcaccgaggactgctaaccaagtgctccctgtgccagcgaactggtgccaccagcagctgcaatcgcatgcgttgccccaatgtctaccattttggttgtgccatccgcgccaagtgcatgttcttcaaggacaagaccatgctgtgtccaatgcataagatcaaggggccctgtgagcaagagctgagctcttttgctgtcttccggcgggtctacattgagcgggacgaggtgaagcaaatcgctagcatcattcagcggggagaacggctgcacatgttccgtgtgggggggcttgtgttccacgccatcggacagctgctgcctcaccagatggctgactttcatagtgccactgccctctatcccgtgggctacgaggccacgcgcatctattggagcctccgcaccaacaatcgtcgctgctgctatcgctgttctattggtgagaacaacgggcggccggagtttgtaatcaaagtcatcgagcagggcctggaggacctggtcttcactgacgcctctccccaggccgtgtggaatcgcatcattgagcctgtggctgccatgagaaaagaggctgacatgctgcgactcttccctgagtatctgaagggcgaggagctctttgggctgacggtgcatgccgtgcttcgcatagctgaatcactgcccggggtggagagctgtcaaaactatttattccgctatgggcgccacccccttatggagctgccactcatgatcaaccccactggctgtgcccgatcagagcctaaaatcctcacacactacaaacggccccataccctgaacagcaccagcatgtctaaggcatatcagagcaccttcacaggcgagaccaacaccccctacagcaagcagtttgtgcactccaagtcatctcagtaccggcggctgcgcaccgaatggaagaacaacgtgtacctggctcgctcccgtatccagggcctggggctctatgcagccaaggacctagaaaagcacacaatggttatcgagtacattggcaccatcattcggaacgaggtggccaaccggcgggagaaaatctacgaagagcagaatcgaggcatctacatgttccgaataaacaatgaacatgtgattgatgctacgttgaccggcggccctgccaggtacattaaccattcctgtgcccctaactgtgtggccgaagtcgtgacatttgacaaagaggacaaaatcatcatcatctccagccggcgaatccccaaaggagaggagctaacctatgactatcagtttgattttgaggacgatcagcacgagatcccctgc

SEQ ID NO: 94 - DNA, 2091 bp, Homo sapiens
gagcggagccgcgggcgggagggcggacggaccgactgacggtagggacgggaggcgagcaagatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctgaatggacctctccagctctggcttcctgctgagtccctcacgtttcttccccaacccctcagattttatattttctatttctctgtgtatttatataaaaatttattaaatataaatatccccagggacagaaaccaaggccccgagctcagggcagctgtgctgggtgagctcttccaggagccaccttgccacccattcttcccgttcttaactttgaaccataaagggtgccaggtctgggtgaaagggatacttttatgcaaccataagacaaactcctgaaatgccaagtgcctgcttagtagctttggaaaggtgcccttattgaacattctagaaggggtggctgggtcttcaaggatctcctgtttttttcaggctcctaaagtaacatcagccatttttagattggttctgttttcgtaccttcccactggcctcaagtgagccaagaaacactgcctgccctctgtctgtcttctcctaattctgcaggtggaggttgctagtctagtttcctttttgagatactattttcatttttgtgagcctctttgtaataaaatggtacatttct

SEQ ID NO: 95 - DNA, 1985 bp, Homo sapiens
cgccgagctttcggcacctctgccgggtggtaccgagccttcccggcgccccctcctctcctcccaccggcctgcccttccccgcgggactatcgcccccacgtttccctcagcccttttctctcccggccgagccgcggcggcagcagcagcagcagcagcagcaggaggaggagcccggtggcggcggtggccggggagcccatggcgtacagtcaaggaggcggcaaaaaaaaagtctgctactactacgacggtgatattggaaattattattatggacagggtcatcccatgaagcctcatagaatccgcatgacccataacttgctgttaaattatggcttatacagaaaaatggaaatatataggccccataaagccactgccgaagaaatgacaaaatatcacagtgatgagtatatcaaatttctacggtcaataagaccagataacatgtctgagtatagtaagcagatgcatatatttaatgttggagaagattgtccagcgtttgatggactctttgagttttgtcagctctcaactggcggttcagttgctggagctgtgaagttaaaccgacaacagactgatatggctgttaattgggctggaggattacatcatgctaagaaatacgaagcatcaggattctgttacgttaatgatattgtgcttgccatccttgaattactaaagtatcatcagagagtcttatatattgatatagatattcatcatggtgatggtgttgaagaagctttttatacaacagatcgtgtaatgacggtatcattccataaatatggggaatactttcctggcacaggagacttgagggatattggtgctggaaaaggcaaatactatgctgtcaattttccaatgtgtgatggtatagatgatgagtcatatgggcagatatttaagcctattatctcaaaggtgatggagatgtatcaacctagtgctgtggtattacagtgtggtgcagactcattatctggtgatagactgggttgtttcaatctaacagtcaaaggtcatgctaaatgtgtagaagttgtaaaaacttttaacttaccattactgatgcttggaggaggtggctacacaatccgtaatgttgctcgatgttggacatatgagactgcagttgcccttgattgtgagattcccaatgagttgccatataatgattactttgagtattttggaccagacttcaaactgcatattagtccttcaaacatgacaaaccagaacactccagaatatatggaaaagataaaacagcgtttgtttgaaaatttgcgcatgttacctcatgcacctggtgtccagatgcaagctattccagaagatgctgttcatgaagacagtggagatgaagatggagaagatccagacaagagaatttctattcgagcatcagacaagcggatagcttgtgatgaagaattctcagattctgaggatgaaggagaaggaggtcgaagaaatgtggctgatcataagaaaggagcaaagaaagctagaattgaagaagataagaaagaaacagaggacaaaaaaacagacgttaaggaagaagataaatccaaggacaacagtggtgaaaaaacagataccaaaggaaccaaatcagaacagctcagcaacccctgaatttgacagtctcaccaatttcagaaaatcattaaaaagaaaatattgaaaggaaaatgttttctttttgaagacttctggcttcattttatactactttggcatggactgtatttattttcaaatgggactttttcgtttttgtttttctgggcaagttttattgtgagattttctaattatgaagcaaaatttcttttctccaccatgctttatgtgatagtatttaaaattgatgtgagttattatgtcaaaaaaactgatctattaaagaagtaattggcctttctgagctgaaaaaaaaaaaaaaaaag

SEQ ID NO: 96 - DNA, 1955 bp, Homo sapiens
gcggcggccgcgggcggcgggcggcggaggtgcggggcctgctcccgccggcaccatggccaagaccgtggcctatttctacgaccccgacgtgggcaacttccactacggagctggacaccctatgaagccccatcgcctggcattgacccatagcctggtcctgcattacggtctctataagaagatgatcgtcttcaagccataccaggcctcccagcatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaagatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatccgccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggccagaggcacccaatgagttctatgatggagaccatgacaatgacaaggaaagcgatgtggagatttaagagtggcttgggatgctgtgtcccaaggaatttcttttcacctcttggttgggctggagggaaaaggagtggctcctagagtcctgggggtcaccccagggcttttgctgactctgggaaagagtctggagaccacatttggttctcgaaccatctacctgcttttcctctctctcccaaggcctgacaatggtacctattagggatggagatacagacaaggatagctatctgggacattattggcagtgggccctggaggccagtccctagccccccttgccccttatttcttccctgcttccctcgaacccagagatttttgagggatgaacgggtagacaaggactgagattgcctctgacttcctcctcccctgggttctgacttcttcctccccttgcttccagggaagatgaagagagagagatttggaaggggctctggctccctaacacctgaatcccagatgatgggaagtatgttttcaagtgtggggaggatatgaaaatgttctgttctcacttttggctttatgtccattttaccactgtttttatccaataaactaagtcggtattttttgtacctttaaaaaaaaaaaaaaaaaaaa

SEQ ID NO: 97 - DNA, 8459 bp, Homo sapiens
ggaggttgtggggccgccgccgcggagcaccgtccccgccgccgcccgagcccgagcccgagcccgcgcacccgcccgcgccgccgccgccgccgcccgaacagcctcccagcctgggcccccggcggcgccgtggccgcgtcccggctgtcgccgcccgagcccgagcccgcgcgccggcgggtggcggcgcaggctgaggagatgcggcgcggagcgccggagcagggctagagccggccgccgccgcccgccgcggtaagcgcagccccggcccggcgcccgcgggccattgtccgccgcccgccccgcgccccgcgcagcctgcaggccttggagcccgcggcaggtggacgccgccggtccacacccgccccgcgcgcggccgtgggaggcgggggccagcgctggccgcgcgccgtgggacccgccggtccccagggccgcccggccccttctggacctttccacccgcgccgcgaggcggcttcgcccgccggggcgggggcgcgggggtgggcacggcaggcagcggcgccgtctcccggtgcggggcccgcgccccccgagcaggttcatctgcagaagccagcggacgcctctgttcaacttgtgggttacctggctcatgagaccttgccggcgaggctcggcgcttgaacgtctgtgacccagccctcaccgtcccggtacttgtatgtgttggtgggagtttggagctcgttggagctatcgtttccgtggaaattttgagccatttcgaatcacttaaaggagtggacattgctagcaatgagctcccaaagccatccagatggactttctggccgagaccagccagtggagctgctgaatcctgcccgcgtgaaccacatgcccagcacggtggatgtggccacggcgctgcctctgcaagtggccccctcggcagtgcccatggacctgcgcctggaccaccagttctcactgcctgtggcagagccggccctgcgggagcagcagctgcagcaggagctcctggcgctcaagcagaagcagcagatccagaggcagatcctcatcgctgagttccagaggcagcacgagcagctctcccggcagcacgaggcgcagctccacgagcacatcaagcaacaacaggagatgctggccatgaagcaccagcaggagctgctggaacaccagcggaagctggagaggcaccgccaggagcaggagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcatttccagcgaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaattacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcagcgcggagaacggtatcgcgcccgccgtccccagcatcccggcggagacgagtttggcgcacagacttgtggcacgagaaggctcggccgctccacttcccctctacacatcgccatccttgcccaacatcacgctgggcctgcctgccaccggcccctctgcgggcacggcgggccagcaggacaccgagagactcacccttcccgccctccagcagaggctctcccttttccccggcacccacctcactccctacctgagcacctcgcccttggagcgggacggaggggcagcgcacagccctcttctgcagcacatggtcttactggagcagccaccggcacaagcacccctcgtcacaggcctgggagcactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgaggaagaggcagagcccccacgggaggtggagccgggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtctgtgcaggagccccccaccaagccgaggttcacgacaggcctcgtgtatgacacgctgatgctgaagcaccagtgcacctgcgggagtagcagcagccaccccgagcacgccgggaggatccagagcatctggtcccgcctgcaggagacgggcctccggggcaaatgcgagtgcatccgcggacgcaaggccaccctggaggagctacagacggtgcactcggaagcccacaccctcctgtatggcacgaaccccctcaaccggcagaaactggacagtaagaaacttctaggctcgctcgcctccgtgttcgtccggctcccttgcggtggtgttggggtggacagtgacaccatatggaacgaggtgcactcggcgggggcagcccgcctggctgtgggctgcgtggtagagctggtcttcaaggtggccacaggggagctgaagaatggctttgctgtggtccgcccccctggacaccatgcggaggagagcacgcccatgggcttttgctacttcaactccgtggccgtggcagccaagcttctgcagcagaggttgagcgtgagcaagatcctcatcgtggactgggacgtgcaccatggaaacgggacccagcaggctttctacagcgaccctagcgtcctgtacatgtccctccaccgctacgacgatgggaacttcttcccaggcagcggggctcctgatgaggtgggcacagggcccggcgtgggtttcaacgtcaacatggctttcaccggcggcctggacccccccatgggagacgctgagtacttggcggccttcagaacggtggtcatgccgatcgccagcgagtttgccccggatgtggtgctggtgtcatcaggcttcgatgccgtggagggccaccccacccctcttgggggctacaacctctccgccagatgcttcgggtacctgacgaagcagctgatgggcctggctggcggccggattgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcggggcgttctctgatcgaggctcagacttgcgagaacgaagaagccgagacggtcaccgccatggcctcgctgtccgtgggcgtgaagcccgccgaaaagagaccagatgaggagcccatggaagaggagccgcccctgtagcactccctcgaagctgctgttctcttgtctgtctgtctctgtcttgaagctcagccaagaaactttcccgtgtcacgcctgcgtcccaccgtggggctctcttggagcacccagggacacccagcgtgcaacagccacgggaagcctttctgccgcccaggcccacaggtctcgagacgcacatgcacgcctgggcgtggcagcctcacagggaacacgggacagacgccggcgacgcgcagacacacggacacgcggaagccaagcacactctggcgggtcccgcaagggacgccgtggaagaaaggagcctgtggcaacaggcggccgagctgccgaattcagttgacacgaggcacagaaaacaaatatcaaagatctaataatacaaaacaaacttgattaaaactggtgcttaaagtttattacccacaactccacagtctctgtgtaaaccactcgactcatcttgtagcttattttttttttaaagaggacgttttctacggctgtggcccgcctctgtgaaccatagcggtgtgcggcggggggtctgcacccgggtgggggacagagggacctttaaagaaaacaaaactggacagaaacaggaatgtgagctgggggagctggcttgagtttctcaaaagccatcggaagatgcgagtttgtgcctttttttttattgctctggtggatttttgtggctgggttttctgaagtctgaggaacaatgccttaagaaaaaacaaacagcaggaatcggtgggacagtttcctgtggccagccgagcctggcagtgctggcaccgcgagctggcctgacgcctcaagcacgggcaccagccgtcatctccggggccaggggctgcagcccggcggtccctgttttgctttattgctgtttaagaaaaatggaggtagttccaaaaaagtggcaaatcccgttggaggttttgaagtccaacaaattttaaacgaatccaaagtgttctcacacgtcacatacgattgagcatctccatctggtcgtgaagcatgtggtaggcacacttgcagtgttacgatcggaatgctttttattaaaagcaagtagcatgaagtattgcttaaattttaggtataaataaatatatatatgtataatatatattccaatgtattccaagctaagaaacttacttgattcttatgaaatcttgataaaatatttataatgcatttatagaaaaagtatatatatatatataaaatgaatgcagattgcgaaggtccctgcaaatggatggcttgtgaatttgctctcaaggtgcttatggaaagggatcctgattgattgaaattcatgttttctcaagctccagattggctagatttcagatcgccaacacattcgccactgggcaactaccctacaagtttgtactttcattttaattattttctaacagaaccgctcccgtctccaagccttcatgcacatatgtacctaatgagtttttatagcaaagaatataaatttgctgttgatttttgtatgaattttttcacaaaaagatcctgaataagcattgttttatgaattttacatttttcctcaccatttagcaattttctgaatggtaataatgtctaaatctttttcctttctgaattcttgcttgtacatttttttttacctttcaaaggtttttaattatttttgtttttatttttgtacgatgagttttctgcagcgtacagaattgttgctgtcagattctattttcagaaagtgagaggagggaccgtaggtcttttcggagtgacaccaacgattgtgtctttcctggtctgtcctaggagctgtataaagaagcccaggggctctttttaactttcaacactagtagtattacgaggggtggtgtgtttttcccctccgtggcaagggcagggagggttgcttaggatgcccggccaccctgggaggcttgccagatgccgggggcagtcagcattaatgaaactcatgtttaaacttctctgaccacatcgtcaggatagaattctaacttgagttttccaaagaccttttgagcatgtcagcaatgcatggggcacacgtggggctctttacccacttgggtttttccactgcagccacgtggccagccctggattttggagcctgtggctgcaaggaacccagggacccttgttgcctggtgaacctgcagggagggtatgattgcctgaccaggacagccagtctttactctttttctcttcaacagtaactgacagtcacgttttactggtaacttattttccagcacatgaagccaccagtttcattccaaagtgtatattgggttcagacttgggggcagaagttcagacacaccgtgctcaggagggacccagagccgagtttcggagtttggtaaagtttacagggtagcttctgaaattaactcaaacttttgaccaaatgagtgcagattcttggattcacttggtcactgggctgctgatggtcagctctgagacagtggtttgagagcaggcagaacggtcttgggacttgtttgactttcccctccctggtggccactctttgctctgaagcccagattggcaagaggagctggtccattccccattcatggcacagagcagtggcagggcccagctagcaggctcttctggcctccttggcctcattctctgcatagccctctggggatcctgccacctgccctcttaccccgccgtggcttatggggaggaatgcatcatctcactttttttttttaagcagatgatgggataacatggactgctcagtggccaggttatcagtggggggacttaattctaatctcattcaaatggagacgccctctgcaaaggcctggcagggggaggcacgtttcatctgtcagctcactccagcttcacaaatgtgctgagagcattactgtgtagccttttctttgaagacacactcggctcttctccacagcaagcgtccagggcagatggcagaggatctgcctcggcgtctgcaggcgggaccacgtcagggagggttccttcatgtgttctccctgtgggtccttggacctttagcctttttcttcctttgcaaaggccttgggggcactggctgggagtcagcaagcgagcactttatatccctttgagggaaaccctgatgacgccactgggcctcttggcgtctgccctgccctcgcggcttcccgccgtgccgcagcgtgcccacgtgcccacgccccaccagcaggcggctgtcccggaggccgtggcccgctgggactggccgcccctccccagcgtcccagggctctggttctggagggccactttgtcaaggtgtttcagtttttctttacttcttttgaaaatctgtttgcaaggggaaggaccatttcgtaatggtctgacacaaaagcaagtttgatttttgcagcactagcaatggactttgttgtttttctttttgatcagaacattccttctttactggtcacagccacgtgctcattccattcttctttttgtagactttgggcccacgtgttttatgggcattgatacatatataaatatatagatataaatatatatgaatatatttttttaagtttcctacacctggaggttgcatggactgtacgaccggcatgactttatattgtatacagattttgcacgccaaactcggcagctttggggaagaagaaaaatgcctttctgttcccctctcatgacatttgcagatacaaaagatggaaatttttctgtaaaacaaaaccttgaaggagaggagggcggggaagtttgcgtcttattgaacttattcttaagaaattgtactttttattgtaagaaaaataaaaaggactacttaaacatttgtcatattaagaaaaaaagtttatctagcacttgtgacataccaataatagagtttattgtatttatgtggaaacagtgttttagggaaactactcagaattcacagtgaactgcctgtctctctcgagttgatttggaggaattttgttttgttttgttttgtttgtttccttttatctccttccacgggccaggcgagcgccgcccgccctcactggccttgtgacggtttattctgattgagaactgggcggactcgaaagagtccccttttccgcacagctgtgttgactttttaattacttttaggtgatgtatggctaagatttcactttaagcagtcgtgaactgtgcgagcactgtggtttacaattatactttgcatcgaaaggaaaccatttcttcattgtaacgaagctgagcgtgttcttagctcggcctcactttgtctctggcattgattaaaagtctgctattgaaagaaaaag

SEQ ID NO: 98 - DNA, 3449 bp, Homo sapiens
atgaactctcccaacgagtcggatgggatgtcaggtcgggaaccatccttggaaatcctgccgcggacttctctgcacagcatccctgtgacagtggaggtgaagccggtgctgccaagagccatgcccagttccatggggggtgggggtggaggcagccccagccctgtggagctacggggggctctggtgggctctgtggaccccacactgcgggagcagcaactgcagcaggagctcctggcgctcaagcagcagcagcagctgcagaagcagctcctgttcgctgagttccagaaacagcatgaccacctgacaaggcagcatgaggtccagctgcagaagcacctcaagcagcagcaggagatgctggcagccaagcagcagcaggagatgctggcagccaagcggcagcaggagctggagcagcagcggcagcgggagcagcagcggcaggaagagctggagaagcagcggctggagcagcagctgctcatcctgcggaacaaggagaagagcaaagagagtgccattgccagcactgaggtaaagctgaggctccaggaattcctcttgtcgaagtcaaaggagcccacaccaggcggcctcaaccattccctcccacagcaccccaaatgctggggagcccaccatgcttctttggaccagagttcccctccccagagcggcccccctgggacgcctccctcctacaaactgcctttgcctgggccctacgacagtcgagacgacttccccctccgcaaaacagcctctgaacccaacttgaaagtgcgttcaaggctaaaacagaaggtggctgagcggagaagcagtcccctcctgcgtcgcaaggatgggactgttattagcacctttaagaagagagctgttgagatcacaggtgccgggcctggggcgtcgtccgtgtgtaacagcgcacccggctccggccccagctctcccaacagctcccacagcaccatcgctgagaatggctttactggctcagtccccaacatccccactgagatgctccctcagcaccgagccctccctctggacagctcccccaaccagttcagcctctacacgtctccttctctgcccaacatctccctagggctgcaggccacggtcactgtcaccaactcacacctcactgcctccccgaagctgtcgacacagcaggaggccgagaggcaggccctccagtccctgcggcagggtggcacgctgaccggcaagttcatgagcacatcctctattcctggctgcctgctgggcgtggcactggagggcgacgggagcccccacgggcatgcctccctgctgcagcatgtgctgttgctggagcaggcccggcagcagagcaccctcattgctgtgccactccacgggcagtccccactagtgacgggtgaacgtgtggccaccagcatgcggacggtaggcaagctcccgcggcatcggcccctgagccgcactcagtcctcaccgctgccgcagagtccccaggccctgcagcagctggtcatgcaacaacagcaccagcagttcctggagaagcagaagcagcagcagctacagctgggcaagatcctcaccaagacaggggagctgcccaggcagcccaccacccaccctgaggagacagaggaggagctgacggagcagcaggaggtcttgctgggggagggagccctgaccatgccccgggagggctccacagagagtgagagcacacaggaagacctggaggaggaggacgaggaagaggatggggaggaggaggaggattgcatccaggttaaggacgaggagggcgagagtggtgctgaggaggggcccgacttggaggagcctggtgctggatacaaaaaactgttctcagatgcccagccgctgcagcctttgcaggtgtaccaggcgcccctcagcctggccactgtgccccaccaggccctgggccgtacccagtcctcccctgctgcccctgggggcatgaagagccccccagaccagcccgtcaagcacctcttcaccacaggtgtggtctacgacacgttcatgctaaagcaccagtgcatgtgcgggaacacacacgtgcaccctgagcatgctggccggatccagagcatctggtcccggctgcaggagacaggcctgcttagcaagtgcgagcggatccgaggtcgcaaagccacgctagatgagatccagacagtgcactctgaataccacaccctgctctatgggaccagtcccctcaaccggcagaagctagacagcaagaagttgctcggccccatcagccagaagatgtatgctgtgctgccttgtgggggcatcggggtggacagtgacaccgtgtggaatgagatgcactcctccagtgctgtgcgcatggcagtgggctgcctgctggagctggccttcaaggtggctgcaggagagctcaagaatggatttgccatcatccggcccccaggacaccacgccgaggaatccacagccatgggattctgcttcttcaactctgtagccatcaccgcaaaactcctacagcagaagttgaacgtgggcaaggtcctcatcgtggactgggacattcaccatggcaatggcacccagcaggcgttctataatgacccctctgtgctctacatctctctgcatcgctatgacaacgggaacttctttccaggctctggggctcctgaagaggttggtggaggaccaggcgtggggtacaatgtgaacgtggcatggacaggaggtgtggacccccccattggagacgtggagtaccttacagccttcaggacagtggtgatgcccattgcccacgagttctcacctgatgtggtcctagtctccgccgggtttgatgctgttgaaggacatctgtctcctctgggtggctactctgtcaccgccagatgttttggccacttgaccaggcagctgatgaccctggcagggggccgggtggtgctggccctggagggaggccatgacttgaccgccatctgtgatgcctctgaggcttgtgtctcggctctgctcagtgtagagctgcagcccttggatgaggcagtcttgcagcaaaagcccaacatcaacgcagtggccacgctagagaaagtcatcgagatccagagcaaacactggagctgtgtgcagaagttcgccgctggtctgggccggtccctgcgagaggcccaagcaggtgagaccgaggaggccgagactgtgagcgccatggccttgctgtcggtgggggccgagcaggcccaggctgcggcagcccgggaacacagccccaggccggcagaggagcccatggagcaggagcctgccctgtgacgccccggcccccatccctctgggcttcaccattgtgattttgtttattttttctattaaaaacaaaaagtcacacattc

SEQ ID NO: 99 - DNA, 4099 bp, Homo sapiens
gggcagtcccctgaggagcggggctggttgaaacgctaggggcgggatctggcggagtggaagaaccgcggcaggggccaagcctcctcaactatgacctcaaccggccaggattccaccacaaccaggcagcgaagaagtaggcagaacccccagtcgccccctcaggactccagtgtcacttcgaagcgaaatattaaaaagggagccgttccccgctctatccccaatctagcggaggtaaagaagaaaggcaaaatgaagaagctcggccaagcaatggaagaagacctaatcgtgggactgcaagggatggatctgaaccttgaggctgaagcactggctggcactggcttggtgttggatgagcagttaaatgaattccattgcctctgggatgacagcttcccggaaggccctgagcggctccatgccatcaaggagcaactgatccaggagggcctcctagatcgctgcgtgtcctttcaggcccggtttgctgaaaaggaagagctgatgttggttcacagcctagaatatattgatctgatggaaacaacccagtacatgaatgagggagaactccgtgtcctagcagacacctacgactcagtttatctgcatccgaactcatactcctgtgcctgcctggcctcaggctctgtcctcaggctggtggatgcggtcctgggggctgagatccggaatggcatggccatcattaggcctcctggacatcacgcccagcacagtcttatggatggctattgcatgttcaaccacgtggctgtggcagcccgctatgctcaacagaaacaccgcatccggagggtccttatcgtagattgggatgtgcaccacggtcaaggaacacagttcaccttcgaccaggaccccagtgtcctctatttctccatccaccgctacgagcagggtaggttctggccccacctgaaggcctctaactggtccaccacaggtttcggccaaggccaaggatataccatcaatgtgccttggaaccaggtggggatgcgggatgctgactacattgctgctttcctgcacgtcctgctgccagtcgccctcgagttccagcctcagctggtcctggtggctgctggatttgatgccctgcaaggggaccccaagggtgagatggccgccactccggcagggttcgcccagctaacccacctgctcatgggtctggcaggaggcaagctgatcctgtctctggagggtggctacaacctccgcgccctggctgaaggcgtcagtgcttcgctccacacccttctgggagacccttgccccatgctggagtcacctggtgccccctgccggagtgcccaggcttcagtttcctgtgctctggaagcccttgagcccttctgggaggttcttgtgagatcaactgagaccgtggagagggacaacatggaggaggacaatgtagaggagagcgaggaggaaggaccctgggagccccctgtgctcccaatcctgacatggccagtgctacagtctcgcacagggctggtctatgaccaaaatatgatgaatcactgcaacttgtgggacagccaccaccctgaggtaccccagcgcatcttgcggatcatgtgccgtctggaggagctgggccttgccgggcgctgcctcaccctgacaccgcgccctgccacagaggctgagctgctcacctgtcacagtgctgagtacgtgggtcatctccgggccacagagaaaatgaaaacccgggagctgcaccgtgagagttccaactttgactccatctatatctgccccagtaccttcgcctgtgcacagcttgccactggcgctgcctgccgcctggtggaggctgtgctctcaggagaggttctgaatggtgctgctgtggtgcgtcccccaggacaccacgcagagcaggatgcagcttgcggtttttgctttttcaactctgtggctgtggctgctcgccatgcccagactatcagtgggcatgccctacggatcctgattgtggattgggatgtccaccacggtaatggaactcagcacatgtttgaggatgaccccagtgtgctatatgtgtccctgcaccgctatgatcatggcaccttcttccccatgggggatgagggtgccagcagccagatcggccgggctgcgggcacaggcttcaccgtcaacgtggcatggaacgggccccgcatgggtgatgctgactacctagctgcctggcatcgcctggtgcttcccattgcctacgagtttaacccagaactggtgctggtctcagctggctttgatgctgcacggggggatccgctggggggctgccaggtgtcacctgagggttatgcccacctcacccacctgctgatgggccttgccagtggccgcattatccttatcctagagggtggctataacctgacatccatctcagagtccatggctgcctgcactcgctccctccttggagacccaccacccctgctgaccctgccacggcccccactatcaggggccctggcctcaatcactgagaccatccaagtccatcgcagatactggcgcagcttacgggtcatgaaggtagaagacagagaaggaccctccagttctaagttggtcaccaagaaggcaccccaaccagccaaacctaggttagctgagcggatgaccacacgagaaaagaaggttctggaagcaggcatggggaaagtcacctcggcatcatttggggaagagtccactccaggccagactaactcagagacagctgtggtggccctcactcaggaccagccctcagaggcagccacagggggagccactctggcccagaccatttctgaggcagccattgggggagccatgctgggccagaccacctcagaggaggctgtcgggggagccactccggaccagaccacctcagaggagactgtgggaggagccattctggaccagaccacctcagaggatgctgttgggggagccacgctgggccagactacctcagaggaggctgtaggaggagctacactggcccagaccacctcggaggcagccatggagggagccacactggaccagactacgtcagaggaggctccagggggcaccgagctgatccaaactcctctagcctcgagcacagaccaccagacccccccaacctcacctgtgcagggaactacaccccagatatctcccagtacactgattgggagtctcaggaccttggagctaggcagcgaatctcagggggcctcagaatctcaggccccaggagaggagaacctactaggagaggcagctggaggtcaggacatggctgattcgatgctgatgcagggatctaggggcctcactgatcaggccatattttatgctgtgacaccactgccctggtgtccccatttggtggcagtatgccccatacctgcagcaggcctagacgtgacccaaccttgtggggactgtggaacaatccaagagaattgggtgtgtctctcttgctatcaggtctactgtggtcgttacatcaatggccacatgctccaacaccatggaaattctggacacccgctggtcctcagctacatcgacctgtcagcctggtgttactactgtcaggcctatgtccaccaccaggctctcctagatgtgaagaacatcgcccaccagaacaagtttggggaggatatgccccacccacactaagccccagaatacggtccctcttcaccttctgaggcccacgatagaccagctgtagctcattccagcctgtaccttggatgaggggtagcctcccactgcatcccatcctgaatatcctttgcaactccccaagagtgcttatttaagtgttaatacttttaagagaactgcgacgattaattgtggatctccccctgcccattgcctgcttgaggggcaccactactccagcccagaaggaaaggggggcagctcagtggccccaagagggagctgatatcatgaggataacattggcgggaggggagttaactggcaggcatggcaaggttgcatatgtaataaagtacaagctgtt

SEQ ID NO: 100 - DNA, 4206 bp, Homo sapiens
ataatacctaccttgcaggaccacgacaggattaagtgaggaaaaacccccatgagagtgttttgccattgtcaagtgagcctgagggaggctgaggggggatcaggctgtatcatgcccccgaggacaaactttccagtttaccctgctccctctctctgtccctaggctgccccaggccctgtgcagacacaccaggccctcagccgcagcccatggacctgcgggtgggccagcggcccccagtggagcccccaccagagcccacattgctggccctgcagcgtccccagcgcctgcaccaccacctcttcctagcaggcctgcagcagcagcgctcggtggagcccatgaggctctccatggacacgccgatgcccgagttgcaggtgggaccccaggaacaagagctgcggcagcttctccacaaggacaagagcaagcgaagtgctgtagccagcagcgtggtcaagcagaagctagcggaggtgattctgaaaaaacagcaggcggccctagaaagaacagtccatcccaacagccccggcattccctacagaaccctggagcccctggagacggaaggagccacccgctccatgctcagcagctttttgcctcctgttcccagcctgcccagtgaccccccagagcacttccctctgcgcaagacagtctctgagcccaacctgaagctgcgctataagcccaagaagtccctggagcggaggaagaatccactgctccgaaaggagagtgcgccccccagcctccggcggcggcccgcagagaccctcggagactcctccccaagtagtagcagcacgcccgcatcagggtgcagctcccccaatgacagcgagcacggccccaatcccatcctgggctcggaggcgctcttgggccagcggctgcggctgcaggagacttctgtggccccgttcgccttgccgacagtgtccttgctgcccgcaatcactctggggctgcccgcccctgccagggctgacagtgaccgcaggacccatccgactctgggccctcgggggccaatcctggggagcccccacactcccctcttcctgccccatggcttggagcccgaggctgggggcaccttgccctctcgcctgcagcccattctcctcctggacccctcaggctctcatgccccgctgctgactgtgcccgggcttgggcccttgcccttccactttgcccagtccttaatgaccaccgagcggctctctgggtcaggcctccactggccactgagccggactcgctcagagcccctgccccccagtgccaccgctcccccaccgccgggccccatgcagccccgcctggagcagctcaaaactcacgtccaggtgatcaagaggtcagccaagccgagtgagaagccccggctgcggcagataccctcggctgaagacctggagacagatggcgggggaccgggccaggtggtggacgatggcctggagcacagggagctgggccatgggcagcctgaggccagaggccccgctcctctccagcagcaccctcaggtgttgctctgggaacagcagcgactggctgggcggctcccccggggcagcaccggggacactgtgctgcttcctctggcccagggtgggcaccggcctctgtcccgggctcagtcttccccagccgcacctgcctcactgtcagccccagagcctgccagccaggcccgagtcctctccagctcagagacccctgccaggaccctgcccttcaccacagggctgatctatgactcggtcatgctgaagcaccagtgctcctgcggtgacaacagcaggcacccggagcacgccggccgcatccagagcatctggtcccggctgcaggagcgggggctccggagccagtgtgagtgtctccgaggccggaaggcctccctggaagagctgcagtcggtccactctgagcggcacgtgctcctctacggcaccaacccgctcagccgcctcaaactggacaacgggaagctggcagggctcctggcacagcggatgtttgtgatgctgccctgtggtggggttggggtggacactgacaccatctggaatgagcttcattcctccaatgcagcccgctgggccgctggcagtgtcactgacctcgccttcaaagtggcttctcgtgagctaaagaatggtttcgctgtggtgcggcccccaggacaccatgcagatcattcaacagccatgggcttctgcttcttcaactcagtggccatcgcctgccggcagctgcaacagcagagcaaggccagcaagatcctcattgtagactgggacgtgcaccatggcaacggcacccagcaaaccttctaccaagaccccagtgtgctctacatctccctgcatcgccatgacgacggcaacttcttcccggggagtggggctgtggatgaggtaggggctggcagcggtgagggcttcaatgtcaatgtggcctgggctggaggtctggacccccccatgggggatcctgagtacctggctgctttcaggatagtcgtgatgcccatcgcccgagagttctctccagacctagtcctggtgtctgctggatttgatgctgctgagggtcacccggccccactgggtggctaccatgtttctgccaaatgttttggatacatgacgcagcaactgatgaacctggcaggaggcgcagtggtgctggccttggagggtggccatgacctcacagccatctgtgacgcctctgaggcctgtgtggctgctcttctgggtaacagggtggatcccctttcagaagaaggctggaaacagaaacccaacctcaatgccatccgctctctggaggccgtgatccgggtgcacagtaaatactggggctgcatgcagcgcctggcctcctgtccagactcctgggtgcctagagtgccaggggctgacaaagaagaagtggaggcagtgaccgcactggcgtccctctctgtgggcatcctggctgaagataggccctcggagcagctggtggaggaggaagaacctatgaatctctaaggctctggaaccatctgcccgcccaccatgcccttgggacctggttctcttctaacccctggcaatagcccccattcctgggtctttagagatcctgtgggcaagtagttggaaccagagaacagcctgcctgctttgacagttatcccagggagcgtgagaaaatccctgggtctagaatgggaactggagaggaccctgagaggagacgggctgggcggcgacccccacagggctctcgagaacagattctcccctccagtatgggccctggctgtggcccccattcctcaggactgcacagaggaggactggctccggctccgtcgggctcacccttaaccactattcctggctctgcaaaccccagactttgcacacagccccaggctccacacagaaatgtgaacttggcctcagacaggctggcccttcctaggctctaggggctaggggggagtggggagccaagaggtcccatattcctgagtgcaggggtagtccctctcacctgcttcctcagacgactctggaagcttccctctaccaccgggcactgagacgaagctccctgacagccgagactggcagccctccatctggtccgtaccctcgccagaggcccccctacatcaacctcctggcgatgccctggtggagcagatgggtgctctgggagtcctgtgcttcctgatccaatggtgccaaacccttcatctcccccagaagcgcagcatacccctgggacccctcggccactgcccactcggggagccttctctgtttctggggcctcccccaccatagctctgattcccaccccacataggaatagcctgactgagggggaaggggtgggagagaagatacagacatggaggaggggaggctgctctggcaaagtcttcaaggcttttgggggtccaggcctggggtcaagaaggaaaatgtgtgtgagcatgtgtgtgagtgaggcgtgtgtgtgagcgtgtgtgtgagtgaggcgtgtgtgtgtgtctttcctaggacccaccataccctgtgtatgtatgcatgtttttgtaaaaaggaagaaaatggaaaaaaatctgaacaataaatgttttatttgctttaaaaaaaaaaaaaaaaa

SEQ ID NO: 101 - DNA, 4086 bp, Homo sapiens
ataatacctaccttgcaggaccacgacaggattaagtgaggaaaaacccccatgagagtgttttgccattgtcaagtgagcctgagggaggctgaggggggatcaggctgtatcatgcccccgaggacaaactttccagtttaccctgctccctctctctgtccctaggctgccccaggccctgtgcagacacaccaggccctcagccgcagcccatggacctgcgggtgggccagcggcccccagtggagcccccaccagagcccacattgctggccctgcagcgtccccagcgcctgcaccaccacctcttcctagcaggcctgcagcagcagcgctcggtggagcccatgaggctctccatggacacgccgatgcccgagttgcaggtgggaccccaggaacaagagctgcggcagcttctccacaaggacaagagcaagcgaagtgctgtagccagcagcgtggtcaagcagaagctagcggaggtgattctgaaaaaacagcaggcggccctagaaagaacagtccatcccaacagccccggcattccctacagaaccctggagcccctggagacggaaggagccacccgctccatgctcagcagctttttgcctcctgttcccagcctgcccagtgaccccccagagcacttccctctgcgcaagacagtctctgagcccaacctgaagctgcgctataagcccaagaagtccctggagcggaggaagaatccactgctccgaaaggagagtgcgccccccagcctccggcggcggcccgcagagaccctcggagactcctccccaagtagtagcagcacgcccgcatcagggtgcagctcccccaatgacagcgagcacggccccaatcccatcctgggcgacagtgaccgcaggacccatccgactctgggccctcgggggccaatcctggggagcccccacactcccctcttcctgccccatggcttggagcccgaggctgggggcaccttgccctctcgcctgcagcccattctcctcctggacccctcaggctctcatgccccgctgctgactgtgcccgggcttgggcccttgcccttccactttgcccagtccttaatgaccaccgagcggctctctgggtcaggcctccactggccactgagccggactcgctcagagcccctgccccccagtgccaccgctcccccaccgccgggccccatgcagccccgcctggagcagctcaaaactcacgtccaggtgatcaagaggtcagccaagccgagtgagaagccccggctgcggcagataccctcggctgaagacctggagacagatggcgggggaccgggccaggtggtggacgatggcctggagcacagggagctgggccatgggcagcctgaggccagaggccccgctcctctccagcagcaccctcaggtgttgctctgggaacagcagcgactggctgggcggctcccccggggcagcaccggggacactgtgctgcttcctctggcccagggtgggcaccggcctctgtcccgggctcagtcttccccagccgcacctgcctcactgtcagccccagagcctgccagccaggcccgagtcctctccagctcagagacccctgccaggaccctgcccttcaccacagggctgatctatgactcggtcatgctgaagcaccagtgctcctgcggtgacaacagcaggcacccggagcacgccggccgcatccagagcatctggtcccggctgcaggagcgggggctccggagccagtgtgagtgtctccgaggccggaaggcctccctggaagagctgcagtcggtccactctgagcggcacgtgctcctctacggcaccaacccgctcagccgcctcaaactggacaacgggaagctggcagggctcctggcacagcggatgtttgtgatgctgccctgtggtggggttggggtggacactgacaccatctggaatgagcttcattcctccaatgcagcccgctgggccgctggcagtgtcactgacctcgccttcaaagtggcttctcgtgagctaaagaatggtttcgctgtggtgcggcccccaggacaccatgcagatcattcaacagccatgggcttctgcttcttcaactcagtggccatcgcctgccggcagctgcaacagcagagcaaggccagcaagatcctcattgtagactgggacgtgcaccatggcaacggcacccagcaaaccttctaccaagaccccagtgtgctctacatctccctgcatcgccatgacgacggcaacttcttcccggggagtggggctgtggatgaggtaggggctggcagcggtgagggcttcaatgtcaatgtggcctgggctggaggtctggacccccccatgggggatcctgagtacctggctgctttcaggatagtcgtgatgcccatcgcccgagagttctctccagacctagtcctggtgtctgctggatttgatgctgctgagggtcacccggccccactgggtggctaccatgtttctgccaaatgttttggatacatgacgcagcaactgatgaacctggcaggaggcgcagtggtgctggccttggagggtggccatgacctcacagccatctgtgacgcctctgaggcctgtgtggctgctcttctgggtaacagggtggatcccctttcagaagaaggctggaaacagaaacccaacctcaatgccatccgctctctggaggccgtgatccgggtgcacagtaaatactggggctgcatgcagcgcctggcctcctgtccagactcctgggtgcctagagtgccaggggctgacaaagaagaagtggaggcagtgaccgcactggcgtccctctctgtgggcatcctggctgaagataggccctcggagcagctggtggaggaggaagaacctatgaatctctaaggctctggaaccatctgcccgcccaccatgcccttgggacctggttctcttctaacccctggcaatagcccccattcctgggtctttagagatcctgtgggcaagtagttggaaccagagaacagcctgcctgctttgacagttatcccagggagcgtgagaaaatccctgggtctagaatgggaactggagaggaccctgagaggagacgggctgggcggcgacccccacagggctctcgagaacagattctcccctccagtatgggccctggctgtggcccccattcctcaggactgcacagaggaggactggctccggctccgtcgggctcacccttaaccactattcctggctctgcaaaccccagactttgcacacagccccaggctccacacagaaatgtgaacttggcctcagacaggctggcccttcctaggctctaggggctaggggggagtggggagccaagaggtcccatattcctgagtgcaggggtagtccctctcacctgcttcctcagacgactctggaagcttccctctaccaccgggcactgagacgaagctccctgacagccgagactggcagccctccatctggtccgtaccctcgccagaggcccccctacatcaacctcctggcgatgccctggtggagcagatgggtgctctgggagtcctgtgcttcctgatccaatggtgccaaacccttcatctcccccagaagcgcagcatacccctgggacccctcggccactgcccactcggggagccttctctgtttctggggcctcccccaccatagctctgattcccaccccacataggaatagcctgactgagggggaaggggtgggagagaagatacagacatggaggaggggaggctgctctggcaaagtcttcaaggcttttgggggtccaggcctggggtcaagaaggaaaatgtgtgtgagcatgtgtgtgagtgaggcgtgtgtgtgagcgtgtgtgtgagtgaggcgtgtgtgtgtgtctttcctaggacccaccataccctgtgtatgtatgcatgtttttgtaaaaaggaagaaaatggaaaaaaatctgaacaataaatgttttatttgctttaaaaaaaaaaaaaaaaa

SEQ ID NO: 102 - DNA, 4238 bp, Homo sapiens
ggggaagagaggcacagacacagataggagaagggcaccggctggagccacttgcaggactgagggtttttgcaacaaaaccctagcagcctgaagaactctaagccagatggggtggctggacgagagcagctcttggctcagcaaagaatgcacagtatgatcagctcagtggatgtgaagtcagaagttcctgtgggcctggagcccatctcacctttagacctaaggacagacctcaggatgatgatgcccgtggtggaccctgttgtccgtgagaagcaattgcagcaggaattacttcttatccagcagcagcaacaaatccagaagcagcttctgatagcagagtttcagaaacagcatgagaacttgacacggcagcaccaggctcagcttcaggagcatatcaaggaacttctagccataaaacagcaacaagaactcctagaaaaggagcagaaactggagcagcagaggcaagaacaggaagtagagaggcatcgcagagaacagcagcttcctcctctcagaggcaaagatagaggacgagaaagggcagtggcaagtacagaagtaaagcagaagcttcaagagttcctactgagtaaatcagcaacgaaagacactccaactaatggaaaaaatcattccgtgagccgccatcccaagctctggtacacggctgcccaccacacatcattggatcaaagctctccaccccttagtggaacatctccatcctacaagtacacattaccaggagcacaagatgcaaaggatgatttcccccttcgaaaaactgcctctgagcccaacttgaaggtgcggtccaggttaaaacagaaagtggcagagaggagaagcagccccttactcaggcggaaggatggaaatgttgtcacttcattcaagaagcgaatgtttgaggtgacagaatcctcagtcagtagcagttctccaggctctggtcccagttcaccaaacaatgggccaactggaagtgttactgaaaatgagacttcggttttgccccctacccctcatgccgagcaaatggtttcacagcaacgcattctaattcatgaagattccatgaacctgctaagtctttatacctctccttctttgcccaacattaccttggggcttcccgcagtgccatcccagctcaatgcttcgaattcactcaaagaaaagcagaagtgtgagacgcagacgcttaggcaaggtgttcctctgcctgggcagtatggaggcagcatcccggcatcttccagccaccctcatgttactttagagggaaagccacccaacagcagccaccaggctctcctgcagcatttattattgaaagaacaaatgcgacagcaaaagcttcttgtagctggtggagttcccttacatcctcagtctcccttggcaacaaaagagagaatttcacctggcattagaggtacccacaaattgccccgtcacagacccctgaaccgaacccagtctgcacctttgcctcagagcacgttggctcagctggtcattcaacagcaacaccagcaattcttggagaagcagaagcaataccagcagcagatccacatgaacaaactgctttcgaaatctattgaacaactgaagcaaccaggcagtcaccttgaggaagcagaggaagagcttcagggggaccaggcgatgcaggaagacagagcgccctctagtggcaacagcactaggagcgacagcagtgcttgtgtggatgacacactgggacaagttggggctgtgaaggtcaaggaggaaccagtggacagtgatgaagatgctcagatccaggaaatggaatctggggagcaggctgcttttatgcaacaggtaataggcaaagatttagctccaggatttgtaattaaagtcattatctgaacatgaaatgcattgcaggtttggtaaatggatatgatttcctatcagtttatatttctctatgatttgagttcagtgtttaaggattctacctaatgcagatatatgtatatatctatatagaggtctttctatatactgatctctatatagatatcaatgtttcattgaaaatccactggtaaggaaatacctgttatactaaaattatgatacataatatctgagcagttaataggctttaaatttatcccaaagcctgctacaccaattacttctaaagaaaacaaattcactgttattttgagtttatgtgttgagatcagtgactgctggatagtctcccagtctgatcaatgaagcattcgattagtttttgattttttgcaacatctagaatttaattttcacatcactgtacataatgtatcatactatagtcttgaacactgttaaaggtagtctgccccttccttcctctctctttttttagttaagtagaaatgttctggtcaccatgccagtagtcctaggttattgtgtaggttgcaattgaacatattaggaatacaggtggttttaaatatatagatgcaaattgcagcactactttaaatattagattatgtctcacatagcactgctcattttacttttattttgtgtaatttgatgacactgtctatcaaaaaagagcaaatgaagcagatgcaaatgttagtgagaagtaatgtgcagcattatggtccaatcagatacaatattgtgtctacaattgcaaaaaacacagtaacaggatgaatattatctgatatcaagtcaaaatcagtttgaaaagaaggtgtatcatattttatattgtcactagaatctcttaagtataattccataatgacatgggcatataccgtaacattctggcaaataacaattagaaaagataggtttaacaaaaaaatttacttgtatataatgcaccttcaggaggactatgtcctttgatgctataaaatacaaacaactttgaaggcaacagaagacactgtttattcaagtcagttctttgtcaggttcctgctgttctcctacagaaaagtgattctgtgagggtgaacaggaaatgccttgtggaaacaggaagtccaagtgattcatgtactgaggaatgtaggaaaaaaaatctgaggatagtgctttactctttctgtttttaaagggcactctatgaattgatttattgtctaagaaaataacaccacaagtagggaaattgttacggaagcttttcactggaacatttccttcatattcccttttgatatgtttaccttgttttataggtttacttttgttaagctagttaaaggttcgttgtattaagacccctttaatatggataatccaaattgacctagaatctttgtgaggttttttctattaaaatatttatatttctaaatccgaggtatttcaaggtgtagtatcctatttcaaaggagatatagcagttttgccaaatgtagacattgttcaactgtatgttattggcacgtgttgtttacattttgctgtgacatttaaaaatatttctttaaaaatgttactgctaaagatacattatccttttttaaaaagtctccattcaaattaaattaacataactagaagttagaaagtttaaaagttttccacataatgaaagtccttctgataatttgacaaatagctataataggaacactccctatcaccaacatattttggttagtatattccttcatattaaaatgactttttgtcagttgttttgcattaaaaatatggcatgcctaagataaaattgtatattttttccatctcataaatattcattttcttcaaagtcttttttcaatctcataaaaaagggatagtgcatcttttaaaatacattttatttggggaggaacatgtggctgagcagacttttgtataatattacttcaaagatatgtaatcacaaacaaaaaaaactattttttataatgtcatttgagagagtttcatcagtacagttggtggacgttaattgtttgaatttgatagtctttgaatttaatcaagaaactacctggaaccagtgaaaaggaaagctggacttaaataatcttagaattaattgataaatgtctcttttaaaatctactgtatttattataatttacacccttgaaggtgatctcttgttttgtgttgtaaatatattgtttgtatgtttcccttcttgccttctgttataagtctcttcctttctcaaataaagttttttttaaaag

SEQ ID NO: 103 - DNA, 1743 bp, Homo sapiens
gccagatctggaaggtggctgcggaacggttttaagcggaagatggaggagccggaggaaccggcggacagtgggcagtcgctggtcccggtttatatctatagtcccgagtatgtcagtatgtgtgactccctggccaagatccccaaacgggccagtatggtgcattctttgattgaagcatatgcactgcataagcagatgaggatagttaagcctaaagtggcctccatggaggagatggccaccttccacactgatgcttatctgcagcatctccagaaggtcagccaagagggcgatgatgatcatccggactccatagaatatgggctaggttatgactgcccagccactgaagggatatttgactatgcagcagctataggaggggctacgatcacagctgcccaatgcctgattgacggaatgtgcaaagtagcaattaactggtctggagggtggcatcatgcaaagaaagatgaagcatctggtttttgttatctcaatgatgctgtcctgggaatattacgattgcgacggaaatttgagcgtattctctacgtggatttggatctgcaccatggagatggtgtagaagacgcattcagtttcacctccaaagtcatgaccgtgtccctgcacaaattctccccaggatttttcccaggaacaggtgacgtgtctgatgttggcctagggaagggacggtactacagtgtaaatgtgcccattcaggatggcatacaagatgaaaaatattaccagatctgtgaaagtgtactaaaggaagtataccaagcctttaatcccaaagcagtggtcttacagctgggagctgacacaatagctggggatcccatgtgctcctttaacatgactccagtgggaattggcaagtgtcttaagtacatccttcaatggcagttggcaacactcattttgggaggaggaggctataaccttgccaacacggctcgatgctggacatacttgaccggggtcatcctagggaaaacactatcctctgagatcccagatcatgagtttttcacagcatatggtcctgattatgtgctggaaatcacgccaagctgccggccagaccgcaatgagccccaccgaatccaacaaatcctcaactacatcaaagggaatctgaagcatgtggtctagttgacagaaagagatcaggtttccagagctgaggagtggtgcctataatgaagacagcgtgtttatgcaagcagtttgtggaatttgtgactgcagggaaaatttgaaagaaattacttcctgaaaatttccaaggggcatcaagtggcagctggcttcctggggtgaagaggcaggcaccccagagtcctcaactggacctaggggaagaaggagatatcccacatttaaagttcttatttaaaaaaacacacacacacaaatgaaatttttaatctttgaaaattatttttaagcgaattggggaggggagtattttaatcatcttaaatgaaacagatcagaagctggatgagagcagtcaccagtttgtagggcaggaggcagctgagaggcagggtttgggcctcaggaccatccaggtggagccctgggagagagggtactgatcagcagactgggaggtggggagaagtccgctggtgttgttttagtgttatatatctttggtttttttaataaaatctttgaaaacctaaaaaaaaaaaaaaaaaaaaaaaaaaaaaa

SEQ ID NO: 104 - DNA, 1963 bp, Homo sapiens
gtgttgtacgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagacccctctcaccctctggagacccaggcagggaaggtgcaggaggctcaggactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaac

SEQ ID NO: 105 - DNA, 1931 bp, Homo sapiens
cgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagaccgactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaacaaaaaaaaaaaaaaaaaaaaaaa

SEQ ID NO: 106 - DNA, 1927 bp, Homo sapiens
ggcgccgggggcgggggtgggaggcggaggcggggccggggcgccgcgggcggggcgccgggggcggggcgagtccggaggactcctcggactgcgcggaacatggcgttctggggttggcgcgccgcggcagccctccggctgtggggccgggtagttgaacgggtcgaggccgggggaggcgtggggccgtttcaggcctgcggctgtcggctggtgcttggcggcagggacgatgtgagtgcggggctgagaggcagccatggggcccgcggtgagcccttggacccggcgcgccccttgcagaggcctcccagacccgaggtgcccagggcattccggaggcagccgagggcagcagctcccagtttcttcttttcgagtattaaaggtggaagaaggtccatatctttttctgtgggtgcttcaagtgttgttggaagtggaggcagcagtgacaaggggaagctttccctgcaggatgtagctgagctgattcgggccagagcctgccagagggtggtggtcatggtgggggccggcatcagcacacccagtggcattccagacttcagatcgccggggagtggcctgtacagcaacctccagcagtacgatctcccgtaccccgaggccatttttgaactcccattcttctttcacaaccccaagccctttttcactttggccaaggagctgtaccctggaaactacaagcccaacgtcactcactactttctccggctgcttcatgacaaggggctgcttctgcggctctacacgcagaacatcgatgggcttgagagagtgtcgggcatccctgcctcaaagctggttgaagctcatggaacctttgcctctgccacctgcacagtctgccaaagacccttcccaggggaggacattcgggctgacgtgatggcagacagggttccccgctgcccggtctgcaccggcgttgtgaagcccgacattgtgttctttggggagccgctgccccagaggttcttgctgcatgtggttgatttccccatggcagatctgctgctcatccttgggacctccctggaggtggagccttttgccagcttgaccgaggccgtgcggagctcagttccccgactgctcatcaaccgggacttggtggggcccttggcttggcatcctcgcagcagggacgtggcccagctgggggacgtggttcacggcgtggaaagcctagtggagcttctgggctggacagaagagatgcgggaccttgtgcagcgggaaactgggaagcttgatggaccagacaaataggatgatggctgcccccacacaataaatggtaacataggagacatccacatcccaattctgacaagacctcatgcctgaagacagcttgggcaggtgaaaccagaatatgtgaactgagtggacacccgaggctgccactggaatgtcttctcaggccatgagctgcagtgactggtagggctgtgtttacagtcagggccaccccgtcacatatacaaaggagctgcctgcctgtttgctgtgttgaactcttcactctgctgaagctcctaatggaaaaagctttcttctgactgtgaccctcttgaactgaatcagaccaactggaatcccagaccgagtctgctttctgtgcctagttgaacggcaagctcggcatctgttggttacaagatccagacttgggccgagcggtccccagccctcttcatgttccgaagtgtagtcttgaggccctggtgccgcacttctagcatgttggtctcctttagtggggctatttttaatgagagaaaatctgttctttccagcatgaaatacatttagtctcctcaaagggaaaaaaaaaaaaaaaaaaaaaaaaaaaaaaaaaaaaaaaaaaaaaaaaaaaaaaaaaaaaaaaaaa

SEQ ID NO: 107 - DNA, 1174 bp, Homo sapiens
gtccgtagagctgtgagagaatgaagatgagctttgcgttgactttcaggtcagcaaaaggccgttggatcgcaaaccccagccagccgtgctcgaaagcctccattgggttatttgtgccagcaagtcctcctctggaccctgagaaggtcaaagagttacagcgcttcatcaccctttccaagagactccttgtgatgactggggcaggaatctccaccgaatcggggataccagactacaggtcagaaaaagtggggctttatgcccgcactgaccgcaggcccatccagcatggtgattttgtccggagtgccccaatccgccagcggtactgggcgagaaacttcgtaggctggcctcaattctcctcccaccagcctaaccctgcacactgggctttgagcacctgggagaaactcggaaagctgtactggttggtgacccaaaatgtggatgctttgcacaccaaggcggggagtcggcgcctgacagagctccacggatgcatggacagggtcctgtgcttggattgtggggaacagactccccggggggtgctgcaagagcgtttccaagtcctgaaccccacctggagtgctgaggcccatggcctggctcctgatggtgacgtctttctctcagaggagcaagtccggagctttcaggtcccaacctgcgttcaatgtggaggccatctgaaaccagatgtcgttttcttcggggacacagtgaaccctgacaaggttgattttgtgcacaagcgtgtaaaagaagccgactccctcttggtggtgggatcatccttgcaggtatactctggttacaggtttatcctcactgcctgggagaagaagctcccgattgcaatactgaacattgggcccacacggtcggatgacttggcgtgtctgaaactgaattctcgttgtggagagttgctgcctttgatagacccatgctgaccacagcctgatattccagaacctggaacagggactttcacttgaatcttgctgctaaatgtaaatgccttctcaaatgacagattccagttcccattcaacagagtagggtgcactgacaaagtatagaaggttctaggtatcttaatgtgtggatattcttaattaaaactcattttttttaaataaaaaattgttcagctttaaaa

SEQ ID NO: 108 - DNA, 1670 bp, Homo sapiens
ccggagcgcggtcgggacacagcgcctctaggagaaagcctggaaggcgctccgggggtacccagagctcttagcgggccggcagcatgtgcggggcccaagtaaatggaaatgttttctaacatataaaaacctacagaagaagaaaataattttctggatcaaattagaagtctgtattatattgatgtctccagattcaaatatattagaaagcagccgtggagacaaccatcttcattttgggagaaataactaaagcccgcctcaagcattagaactacagacaaaccctgatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcaggtttcatttccagggaccctgtggaacgactcttcctgaagcccttgcctgtcatgaaaatgaaactgtttcttaagtgtcctggggaagaaagaaattacagtatatctaagaactaggccacacgcagaggagaaatggtcttatgggtggtgagctgagtactgaacaatctaaaaatagcctctgattccctcgctggaatccaacctgttgataagtgatgggggtttagaagtagcaaagagcacccacattcaaaagtcacagaactggaaagttaattcatattatttggtttgaactgaaacgtgaggtatctttgatgtgtatggttggttattgggagggaaaaattttgtaaattagattgtctaaaaaaaatagttattctgattatatttttgttatctgggcaaagtagaagtcaaggggtaaaaaccctactattctgatttttgcacaagttttagtggaaaataaaatcacactctacagtaaaaaaaaaaaaaaaaaa

SEQ ID NO: 109 - DNA, 2350 bp, Homo sapiens
attcgggggcgcgagctgccccagtaaatggaaatgttttctaacatataaaaacctacagaagaagaaaataattttctggatcaaattagaagtctgtattatattgatgtctccagattcaaatatattagaaagcagccgtggagacaaccatcttcattttgggcgaaataactaaagcccgcctcaagcattagaactacagacaaaccctgatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcagtcatttgatctccatctcatctctaattattataaagaattaaaacaagtcatcattgtagaaaagcaagaaaatgcagatagagaaaaagaagaaaataaaactggagtatttccacaacccaagtttagagttggcccccacctcccatgccatggactgagcagcaggggcccagcatcccttggatatggtggctgtgtcttcatgtgaaagaaactgaacttggtggtttttcctgccagttcaggagagattcttggcatgtaatatatatcactgctcaagtcaagcctcctaaaaccacagacctgtttcagctgctacttcagccaaaattcttcagcttcatattgtcttgaaaacctatgattgtctctaacaaacaggctacttgctagttagaaattcttatcaatttggcaagctacttatcaaccagactgaccacaagaactgtcatctcatcaatgaaggagtaactgatcaatgaagccagcaatgcttttttcttggcatcatcaaagctgacatttagaagagatgctggtgatagtcatctcatcctactcaatttttcaaaggcagaaaccaaccctggagcaattgagaggactgtttaaacacagagcttaacaatggcagaattgtatatctcgtgcttaacagattttggttgaactttaccctaggtcaggggtcagcaaactactgcctgtgggccaaatttgcccaccacctgtatctgtaaataaggtttcattggaacacagctgtggccatatgtttgtatattgtgtgtggctgcttttgcattaggatgacagaggtgaatagttgcaacagagactggctggtctgcaaagcctaaaatatgtcctgtgtggccctttacagaaaaagttttctaacccctgctctaggttacggagaaaaaaaaatggaataatgttctctgctacttttaacctgattttctttgtacctaaataggcagctagaatgctgcctatattttaataaggatttggatctcacaagacaccttaggcctacacaagttgttcagattctttgccccagttctaatctagtgacaaaggcatagaattctcctcccacaggaatgtatttctattttcaaggtgttaattagttccagttttggttttgtcgttttccccatgtccgatgcttatattggatgatttctgataaacctgactattccaataaaccctaggcatttttgaatttaaaaaaaaaaaaaaaaaa

SEQ ID NO: 110 - DNA, 1718 bp, Homo sapiens
gcggaagcggaagagcaggtctccaggggagcgatggcagccgggggtctgagccgctccgagcgcaaagcggcggagcgggtccggaggttgcgggaggagcagcagagggagcgcctccgccaggtgtcgcgcatcctgaggaaggcggcggcggagcgcagcgccgaggagggccggctgctggccgagagcgcggacctggtaacggagctgcagggccggagccggcggcgcgagggcctgaagcggcggcaggaggaggtgtgcgacgacccggaggagctgcgggggaaggtccgggagctggccagcgccgtccggaacgccaaatacttggtcgtctacacaggcgcgggaatcagcacggcagcgtctatcccagactaccggggccctaatggagtgtggacactgcttcagaaagggagaagcgttagtgctgccgacctgagcgaggccgagccaaccctcacccacatgagcatcacccgtctgcatgagcagaagctggtgcagcatgtggtgtctcagaactgtgacgggctccacctgaggagtgggctgccgcgcacggccatctccgagctccacgggaacatgtacattgaagtctgtacctcctgcgttcccaacagggagtacgtgcgggtgttcgatgtgacggagcgcactgccctccacagacaccagacaggccggacctgccacaagtgtgggacccagctgcgggacaccattgtgcactttggggagagggggacgttggggcagcctctgaactgggaagcggcgaccgaggctgccagcagagcagacaccatcctgtgtctagggtccagcctgaaggttctaaagaagtacccacgcctctggtgcatgaccaagccccctagccggcggccgaagctttacatcgtgaacctgcagtggaccccgaaggatgactgggctgccctgaagctacatgggaagtgtgatgacgtcatgcggctcctcatggccgagctgggcttggagatccccgcctatagcaggtggcaggatcccattttctcactggcgactcccctgcgtgctggtgaagaaggcagccacagtcggaagtcgctgtgcagaagcagagaggaggccccgcctggggaccggggtgcaccgcttagctcggcccccatcctagggggctggtttggcaggggctgcacaaaacgcacaaaaaggaagaaagtgacgtaatcacgtgctcgatgaagaacagttggcactttgcagatggccagtgtcacggtgaaggctgggttgcccccacgggtctagggagaacgaactctttggggatgacattttcaccgtgacatttttagccatttgtccttgaggaagccccttgcactgctgcggttgtaccctgatacggcctggccatcgaggacacctgcccatccggcctctgtgtcaagaggtggcagccgcacctttctgtgagaacggaactcgggttatttcagccccggcctgcagagtggaagcgcccagcggcctttcctcgctcaccaggccagtctcagggcctcaccgtatttctactactacttaatgaaaaagtgtgaactttatagaatcctctctgtactggatgtgcggcagaggggtggctccgagcctcggctctatgcagacctttttatttctattaaacgtttctgcactggcaaaaa

SEQ ID NO: 111 - DNA, 10182 bp, Homo sapiens
ccggaaaatggccgccgccgccgccgccgcgccgagcggaggaggaggaggaggcgaggaggagagactgctccataaaaatacagactcaccagttcctgctttgatgtgacatgtgactccccagaatacaccttgcttctgtagaccagctccaacaggattccatggtagctgggatgttagggctcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccgtcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaattt

SEQ ID NO: 112 - DNA, 2170 bp, Homo sapiens
caggaagcccacccagccccgccacgcagagcccagaaggaaagaaagcctcatgcctgagccgaggggagcaccatggatctgacaaaaatgggcatgatccagctgcagaaccctagccaccccacggggctactgtgcaaggccaaccagatgcggctggccgggactttgtgcgatgtggtcatcatggtggacagccaggagttccacgcccaccggacggtgctggcctgcaccagcaagatgtttgagatcctcttccaccgcaatagtcaacactatactttggacttcctctcgccaaagaccttccagcagattctggagtatgcatatacagccacgctgcaagccaaggcggaggacctggatgacctgctgtatgcggccgagatcctggagatcgagtacctggaggaacagtgcctgaagatgctggagaccatccaggcctcagacgacaatgacacggaggccaccatggccgatggcggggccgaggaagaagaggaccgcaaggctcggtacctcaagaacatcttcatctcgaagcattccagcgaggagagtgggtatgccagtgtggctggacagagcctccctgggcccatggtggaccagagcccttcagtctccacttcatttggtctttcagccatgagtcccaccaaggctgcagtggacagtttgatgaccataggacagtctctcctgcagggaactcttcagccacctgcagggcccgaggagccaactctggctgggggtgggcggcaccctggggtggctgaggtgaagacggagatgatgcaggtggatgaggtgcccagccaggacagccctggggcagccgagtccagcatctcaggagggatgggggacaaggttgaggaaagaggcaaagaggggcctgggaccccgactcgaagcagcgtcatcaccagtgctagggagctacactatgggcgagaggagagtgccgagcaggtgccacccccagctgaggctggccaggcccccactggccgacctgagcacccagcacccccgcctgagaagcatctgggcatctactccgtgttgcccaaccacaaggctgacgctgtattgagcatgccgtcttccgtgacctctggcctccacgtgcagcctgccctggctgtctccatggacttcagcacctatggggggctgctgccccagggcttcatccagagggagctgttcagcaagctgggggagctggctgtgggcatgaagtcagagagccggaccatcggagagcagtgcagcgtgtgtggggtcgagcttcctgataacgaggctgtggagcagcacaggaagctgcacagtgggatgaagacgtacgggtgcgagctctgcgggaagcggttcctggatagtttgcggctgagaatgcacttactggctcattcagcgggtgccaaagcctttgtctgtgatcagtgcggtgcacagttttcgaaggaggatgccctggagacacacaggcagacccatactggcactgacatggccgtcttctgtctgctgtgtgggaagcgcttccaggcgcagagcgcactgcagcagcacatggaggtccacgcgggcgtgcgcagctacatctgcagtgagtgcaaccgcaccttccccagccacacggctctcaaacgccacctgcgctcacatacaggcgaccacccctacgagtgtgagttctgtggcagctgcttccgggatgagagcacactcaagagccacaaacgcatccacacgggtgagaaaccctacgagtgcaatggctgtgacaagaagttcagcctcaagcatcagctggagacgcactatagggtgcacacaggtgagaagccctttgagtgtaagctctgccaccagcgctcccgggactactcggccatgatcaagcacctgagaacgcacaacggcgcctcgccctaccagtgcaccatctgcacagagtactgccccagcctctcctccatgcagaagcacatgaagggccacaagcccgaggagatcccgcccgactggaggatagagaagacgtacctctacctgtgctatgtgtgaagggaggcccgcggcggtggagccgagcggggagccaggaaagaagagttggagtgagatgaaggaaggacta

SEQ ID NO: 113 - DNA, 1440 bp, Homo sapiens
ggaattccgtagctattgatttcccggatctggtaacatggcaaaagatgccggtctaattgaagccaacggagaactcaaggtcttcatagaccagaaccttagtcccgggaaaggcgtggtgtccctcgtggccgttcacccctccaccgtcaacccgctcgggaagcagctcttgccaaaaacctttggacagtccaatgtcaacattgcccagcaagtggtaattggtacgcctcagagaccggcagcgtcaaacaccctggtggtaggaagcccacacacccccagcactcactttgcctctcagaaccagccttccgactcctcaccttggtctgccgggaagcgcaacaggaaaggagagaagaatggcaagggcctacggcatttctccatgaaggtctgcgagaaggtgcagaggaaagggaccacttcctacaacgaagtggcagacgagctggttgcggagttcagtgctgccgacaaccacatcttaccaaacgagtcagcttatgaccagaaaaacataagacggcgcgtctacgatgccttaaacgtgctaatggccatgaacatcatctccaaggagaagaaggagatcaagtggattggtctgcccaccaactcggctcaggaatgtcagaacttagaggtggaaagacagaggagacttgaaagaataaaacagaaacagtctcaacttcaagaacttattctacagcaaattgccttcaagaacctggtgcagagaaaccggcatgcggagcagcaggccagccggccaccgccacccaactcagtcatccacctgcccttcatcatcgtcaacaccagcaagaagacggtcatcgactgcagcatctccaatgacaaatttgagtatctgtttaattttgacaacacatttgaaatccacgatgacatagaagtgctgaagcggatgggcatggcttgcgggctggagtcggggagctgctctgccgaagaccttaaaatggccagaagtctggtccccaaggctctggagccatacgtgacagaaatggctcagggaactgttggaggcgtgttcatcacgacggcaggttccacgtctaacggcacaaggttctctgccagtgacctgaccaacggtgcagatgggatgctggccacaagctccaatgggtctcagtacagcggctccagggtggagactccggtgtcctacgtcggggaggacgacgaggaggacgatgacttcaacgagaatgacgaggacgactgacgtcctccccacttcagattcggcttcaggaaaacgtttagcgaaaagaaactttttttttaatgtgggttttctgtttccttttggcctagtcccaagaagatattggtaagctattgaatttagatatgcacctctgataagcaaggattgtttcccgtagattagga

SEQ ID NO: 114 - DNA, 1125 bp, Homo sapiens
cccccatgtgacagtgacggggtccccgctccaggagacgctcgagtctgcgtcccggccctcagcactgtccactgtttcggtgccagcagagaccagcaggcccgggacagttggtgtttggccgtgccgctgtctaacttggtgtgcagagtgaattgccgctgccggagcggagagaggcggagcggccaggagagaggggatttctgtcagcgccggcctcgggagctcggagacatgaacggcttcacgcctgacgagatgagccgcggcggggatgcggccgccgcagtggccgcagtggtcgctgccgcggccgccgccgcctcggcggggaacgggaccggcgcgggcaccggggctgaggtgccgggcgcgggggcggtctcagcggctgggcccccgggggcggccgggccgggccccgggcaactgtgctgcctgcgggaggatggtgagcggtgcggccgggcggcaggcaacgccagcttcagcaagaggatccagaagagcatctcccagaagaaggtgaagatcgagctggataagagcgcaaggcatctttacatatgtgattatcataaaaacttaattcagagtgttcgaaacagaagaaagagaaaagggagtgatgatgatggaggtgattcacctgttcaagatattgataccccagaggttgatttataccaattacaagtaaatacacttaggagatacaaaagacacttcaagctaccaaccagaccaggacttaataaagcacaacttgttgagatagttggttgccactttaggtctattccagtgaatgaaaaagacaccttaacatatttcatctactcagtgaagaatgacaagaacaaatcagatctcaaggttgatagtggtgttcactaggagacgtggaattgagactaataacttggatgttaacactgtttactgttttttcacatgtagaaatgttctttgtgtattttttctacagaggattttctctgattttattttctttgtttctgactctaataattagttggaaactcatataaaatgagctttcctaaattaaatctattttaaataaaggttattactattaaaaaaaaaaaaaaaaaa

SEQ ID NO: 115 - DNA, 1293 bp, Homo sapiens
aggtttgaactgactttattattttgtaaatgtgaattttacaaagcgctttacaattaatgatcacatccttttgtttgtcatggatttccactgtctgaaacggctctgagcacgcttgaagccctcggtttccctgttcgcttttgaatgtttcagttttagttattgatacaatgtcagccatggctaaaaagtaacagtcttgactctaccgagtaacagcacaaaaacagagtgagggctcaggaaaacaaaacaaaggcttcctccttaaaaaaaagacaaaaaaaaaaagctaagcatctgtggctgaaatctaactcagtggtactgtgaaaccttcctttacagcacaggaaaatttattttttaacagtcgtgagttacagtactttaacccctaaacagactctttaaaacaaccgtctcctttttaaaagtctcttttttccaaacattccatccgaaggatggatgctctacttgcacccagtgccatccaaatgttcaagtcaaaaatatttatacattttatacttaggcgagcgtctcgcaggccgtaggaggaagatggcggtggagtcgcgcgttacccaggaggaaattaagaaggagccagagaaaccgatcgaccgcgagaagacatgcccactgttgctacgggtcttcaccaccaataacggccgccaccaccgaatggacgagttctcccggggaaatgtaccgtccagcgagttgcagatctacacttggatggatgcaactttgaaagaactgacaagcttagtaaaagaagtctacccagaagctagaaagaagggcactcacttcaattttgcaatcgtttttacagatgttaaaagacctggctatcgagttaaggagattggcagcaccatgtctggcagaaaggggactgatgattccatgaccctgcagtcgcagaagttccagataggagattacttggacatagcaattacccctccaaatcgggcaccacctccttcagggcgcatgagaccatattaaattctatttactatttgttgaatttatttttccgtcagttatgtaaaataaacatactcttcttcctcccctgattattgccattaagcctttaaattctaaacaaattataatgcatcatctatttaggagttagatttggatgtgctattgtatgattacgaatagtctgtatgtttcaagcccttctgtaaaatatgaagaaaagtctcttagcattctgtgtaaaactgtactgttaaatatatgtgtgtaatc

SEQ ID NO: 116 - DNA, 1946 bp, Homo sapiens
gcctcgtcagctgcctgggcgggctgggaggcgcgggttgaaaagtctcgttccaagtttggagagagagagaagagcgcctcagacctcggtacccgcgagcggggaggaggcaggaaagaaggacgcggcgtctggggagcacccaggcagcaagacggggcccgggctttcgacagtggggagtgtgacgcgcttgggaaaggcaggagcgccagcggtcgggctgctcttggctaacgagaggagtccgaggcggcggcgaggggcgaacgacccgacgcaagatggcgagtaaagagatgtttgaagatactgtggaggagcgtgtcatcaatgaagaatataaaatctggaagaagaatacaccgtttctatatgacctggttatgacccatgctcttcagtggcccagtcttaccgttcagtggcttcctgaagtgactaaacctgaaggaaaagattatgcccttcattggctagtgctggggactcatacgtctgatgagcagaatcatctggtggttgctcgagtacatattcccaatgatgatgcacagtttgatgcttcccattgtgacagtgacaagggtgaatttggtggctttggttctgtaacaggaaaaattgaatgtgaaattaaaatcaatcacgaaggagaagtaaaccgtgctcgttacatgccgcagaatcctcacatcattgctacaaaaacaccatcttctgatgtgttggtttttgactatacaaaacaccctgctaaaccagacccaagtggagaatgtaatcctgatctcagattaagaggtcaccagaaggaaggctatggtctctcctggaattcaaatttgagtggacatctcctaagtgcatctgatgaccatactgtttgtctgtgggatataaacgcaggaccaaaagaaggcaaaattgtggatgctaaagccatctttactggccactcagctgttgtagaggatgtggcctggcacctgctgcacgagtcattgtttggatctgttgctgatgatcagaaacttatgatatgggacaccaggtccaataccacctccaagccgagtcacttggtggatgcgcacactgccgaagtcaactgcctctcattcaatccctacagcgaatttattctagccaccggctctgcggataagaccgtagctttatgggatctgcgtaacttaaaattaaaactccataccttcgaatctcataaagatgaaattttccaggtccactggtctccacataatgaaactattctggcttcaagtggtactgaccgccgcctgaatgtgtgggatttaagtaaaattggggaagaacaatcagcagaagatgcagaagatgggcctccagaactcctgtttattcatggaggacacactgctaagatttcagattttagctggaaccccaatgagccttgggtcatttgctcagtgtctgaggataacatcatgcagatatggcaaatggctgaaaatatttacaatgatgaagagtcagatgtcacgacatccgaactggagggacaaggatcttaaacccaaagtacgagaaatgtttctgttgaatgtaatgctacatgaatgcttgatttatcaagcgccaaaaaggcattgtatagtaggaaatgtaagtggggtggcttatggcttctttatcctctgattctagcactttcaagtgagctgttgcgtactgtatcatattgtagctattagggaagagaagaatgttgcttaagaaagaacatcaccattgattttaaatacaagtagcagggtattgcctttgattcaactgttttaagtcctcattttctcaaactaagtgcttgctgttcccaaatatgcaagaataacttttacactttttccttccaacacttcttgattggctttgcagaaataaagttttaaaat

SEQ ID NO: 117 - DNA, 2314 bp, Homo sapiens
cgcgcgcacagagcgagctcttgcagcctccccgcccctcccgcaacgctcgaccccaggattcccccggctcgcctgcccgccatggccgacaaggaagcagccttcgacgacgcagtggaagaacgagtgatcaacgaggaatacaaaatatggaaaaagaacaccccttttctttatgatttggtgatgacccatgctctggagtggcccagcctaactgcccagtggcttccagatgtaaccagaccagaagggaaagatttcagcattcatcgacttgtcctggggacacacacatcggatgaacaaaaccatcttgttatagccagtgtgcagctccctaatgatgatgctcagtttgatgcgtcacactacgacagtgagaaaggagaatttggaggttttggttcagttagtggaaaaattgaaatagaaatcaagatcaaccatgaaggagaagtaaacagggcccgttatatgccccagaacccttgtatcatcgcaacaaagactccttccagtgatgttcttgtctttgactatacaaaacatccttctaaaccagatccttctggagagtgcaacccagacttgcgtctccgtggacatcagaaggaaggctatgggctttcttggaacccaaatctcagtgggcacttacttagtgcttcagatgaccataccatctgcctgtgggacatcagtgccgttccaaaggagggaaaagtggtagatgcgaagaccatctttacagggcatacggcagtagtagaagatgtttcctggcatctactccatgagtctctgtttgggtcagttgctgatgatcagaaacttatgatttgggatactcgttcaaacaatacttccaaaccaagccactcagttgatgctcacactgctgaagtgaactgcctttctttcaatccttatagtgagttcattcttgccacaggatcagctgacaagactgttgccttgtgggatctgagaaatctgaaacttaagttgcattcctttgagtcacataaggatgaaatattccaggttcagtggtcacctcacaatgagactattttagcttccagtggtactgatcgcagactgaatgtctgggatttaagtaaaattggagaggaacaatccccagaagatgcagaagacgggccaccagagttgttgtttattcatggtggtcatactgccaagatatctgatttctcctggaatcccaatgaaccttgggtgatttgttctgtatcagaagacaatatcatgcaagtgtggcaaatggcagagaacatttataatgatgaagaccctgaaggaagcgtggatccagaaggacaagggtcctagatatgtctttacttgttgtgattttagactccccttttttcttctcaaccctgagagtgatttaacactggttttgagacagactttattcagctatccctctatataataggtaccaccgataatgctattagcccaaaccgtgggtttttctaaatattaataggggggcttgattcaacaaagccacagacttaacgttgaaattttcttcaggaattttctagtaacccaggtctaaagtagctacagaaaggggaatattatgtgtgattatttttcttcttatgctatatccccaagtttttcagactcatttaagtaaaggctagagtgagtaaggaatagagccaaatgaggtaggtgtctgagccatgaagtataaatactgaaagatgtcacttttattcaggaaatagggggagttcaagtcgtatagattcctactcgaaaatcttgacacctgactttccaggatgcacattttcatacgtagaccagtttcctcttggtttcttcagttaagtcaaaacaacacgttcctctttccccatatattcatatatttttgctcgttagtgtatttcttgagctgttttcatgttgtttatttcctgtctgtgaaatggtgtttttttttttgttgttggttttttttttttttttttaacttgggaccaccaagttgtaaagatgtatgtttttacctgacagttataccacaggtagactgtcaagttgagaagagtgaatcaataacttgtatttgttttaaaaattaaattaatccttgataagagttgctttttttttttaggagttagtccttgaccactagtttgatgccatctccattttgggtgacctgtttcaccagcaggcctgttactctccatgactaactgtgtaagtgcttaaaatggaataaattgcttttctacataaaaaaaaa

SEQ ID NO: 118 - DNA, 4740 bp, Homo sapiens
ttccggtttttctcaggggacgttgaaattatttttgtaacgggagtcgggagaggacggggcgtgccccgcgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcacgagttgacctagatgagatgtcgttcactttactgagctacagaaaaacatacgaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatcgaagattatttttggatcatgataaaactcttcagactgattctatagacagttttgaaacacagagaacaccacgaaaaagtaaccttgatgaagaggtgaatataattcctccacacactccagttaggactgttatgaacactatccaacaattaatgatgattttaaattctgcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtataactttcccaggttctgtttatggccacatttaatatcttcagctctttttgtggatataaaatgtgcagatgcaattgtttgggtgagtcctaagccacttgaaatgttagtcattgttatttatacaagattgaaaatcttgtgtaaatcctgccatttaaaaagttgtagcagattgtttcctcttccaaagtaaaattgctgtgctttatggatagtaagaatggccctagagtgggagtcctgataacccaggcctgtctgactactttgccttcttttgtagcatataggtgatgtttgctcttgtttttattaatttatatgtatatttttttaatttaacatgaacacccttagaaaatgtgtcctatctatcttccaaatgcaatttgattgactgcccattcaccaaaattatcctgaactcttctgcaaaaatggatattattagaaattagaaaaaaattactaattttacacattagattttattttactattggaatctgatatactgtgtgcttgttttataaaattttgcttttaattaaataaaagctggaagcaaagtataaccatatgatactatcatactactgaaacagatttcatacctcagaatgtaaaagaacttactgattattttcttcatccaacttatgtttttaaatgaggattattgatagtactcttggtttttataccattcagatcactgaatttataaagtacccatctagtacttgaaaaagtaaagtgttctgccagatcttaggtatagaggaccctaacacagtatatcccaagtgcactttctaatgtttctgggtcctgaagaattaagatacaaattaattttactccataaacagactgttaattataggagccttaatttttttttcatagagatttgtctaattgcatctcaaaattattctgccctccttaatttgggaaggtttgtgttttctctggaatggtacatgtcttccatgtatcttttgaactggcaattgtctatttatcttttatttttttaagtcagtatggtctaacactggcatgttcaaagccacattatttctagtccaaaattacaagtaatcaagggtcattatgggttaggcattaatgtttctatctgattttgtgcaaaagcttcaaattaaaacagctgcattagaaaaagaggcgcttctcccctcccctacacctaaaggtgtatttaaactatcttgtgtgattaacttatttagagatgctgtaacttaaaataggggatatttaaggtagcttcagctagcttttaggaaaatcactttgtctaactcagaattatttttaaaaagaaatctggtcttgttagaaaacaaaattttattttgtgctcatttaagtttcaaacttactattttgacagttattttgataacaatgacactagaaaacttgactccatttcatcattgtttctgcatgaatatcatacaaatcagttagtttttaggtcaagggcttactatttctgggtcttttgctactaagttcacattagaattagtgccagaattttaggaacttcagagatcgtgtattgagatttcttaaataatgcttcagatattattgctttattgcttttttgtattggttaaaactgtacatttaaaattgctatgttactattttctacaattaatagtttgtctattttaaaataaattagttgttaagagtc

SEQ ID NO: 119 - DNA, 2772 bp, Homo sapiens
ggtgtccggagccgcggacctagagatcccagaagccacagcgcagcggcccggcccgccactatttccaggctctgcggggcaggggccgccgcccaccgcccgccgccatggggctgaaggccgcccagaagacgctgttcccgctgcgctccatcgacgacgtggtgcgcctgtttgctgccgagctgggccgagaggagccggacctggtgctcctttccttggtgctgggcttcgtggagcattttctggctgtcaaccgcgtcatccctaccaacgttcccgagctcaccttccagcccagccccgcccccgacccgcctggcggcctcacctactttcccgtggccgacctgtctatcatcgccgccctctatgcccgcttcaccgcccagatccgaggcgccgtcgacctgtccctctatcctcgagaagggggtgtctccagccgtgagctggtgaagaaggtctccgatgtcatatggaacagcctcagccgctcctacttcaaggatcgggcccacatccagtccctcttcagcttcatcacaggcaccaaattggacagctccggtgtggcctttgctgtggttggggcctgccaggccctgggtctccgggatgtccacctcgccctgtctgaggatcatgcctgggtagtgtttgggcccaatggggagcagacagctgaggtcacctggcacggcaagggcaacgaggaccgcaggggccagacagtcaatgccggtgtggctgagcggagctggctgtacctgaaaggatcatacatgcgctgtgaccgcaagatggaggtggcgttcatggtgtgtgccatcaacccttccattgacctgcacaccgactcgctggagcttctgcagctgcagcagaagctgctctggctgctctatgacctgggacatctggaaaggtaccccatggccttagggaacctggcagatctagaggagctggagcccacccctggccggccagacccactcaccctctaccacaagggcattgcctcagccaagacctactatcgggatgaacacatctacccctacatgtacctggctggctaccactgtcgcaaccgcaatgtgcgggaagccctgcaggcctgggcggacacggccactgtcatccaggactacaactactgccgggaagacgaggagatctacaaggagttctttgaagtagccaatgatgtcatccccaacctgctgaaggaggcagccagcttgctggaggcgggcgaggagcggccgggggagcaaagccagggcacccagagccaaggttccgccctccaggaccctgagtgcttcgcccacctgctgcgattctacgacggcatctgcaaatgggaggagggcagtcccacgcctgtgctgcacgtgggctgggccacctttcttgtgcagtccctaggccgttttgagggacaggtgcggcagaaggtgcgcatagtgagccgagaggccgaggcggccgaggccgaggagccgtggggcgaggaagcccgggaaggccggcggcggggcccacggcgggagtccaagccagaggagcccccgccgcccaagaagccagcactggacaagggcctgggcaccggccagggtgcagtgtcaggacccccccggaagcctcctgggactgtcgctggcacagcccgaggccctgaaggtggcagcacggctcaggtgccagcacccgcagcatcaccaccgccggagggtccagtgctcactttccagagtgagaagatgaagggcatgaaggagctgctggtggccaccaagatcaactcgagcgccatcaagctgcaactcacggcacagtcgcaagtgcagatgaagaagcagaaagtgtccacccctagtgactacactctgtctttcctcaagcggcagcgcaaaggcctctgaactactggggacttcggaccgcttgtggggacccaggctccgccttagtcccccaactctgagcccatgttctgcccccagcccaaaggggacaggcctcacctctacccaaaccctaggttcccggtcccgagtacagtctgtatcaaacccacgattttctccagctcagaacccagggctctgccccagtcgttagaatataggtctcttctcccagaatcccagccggccaatggaaacctcacgctgggtcctaattaccagtctttaaaggcccagcccctagaaacccaagctcctcctcggaaccgctcacctagagccagaccaacgttactcagggctcctcccagcttgtaggagctgaggtttcacccttaacccaagggagcacaggtcccacctccagcccggggagcctaggaccactcagcccctaggagtatatttccgcacttcagaattccatatcttgcgaatccaagctccctgccccaaataacttcagtcctgcttccagaatttggaaatcctagtttcctctccttcgtatcccgagtctgggacacaaaactccgcccccagcctatgagcatcctgagccccgccctcttcctgacgaaactggccccggatcagagcaggacctcccttccgaccctctgggaacctcccagaggtccagcccatctcggagcatcccggaggaaatctgcagaggggttaggagtgggtgacaagagcctgatctcttcctgttttgtacatagatttatttttcagttccaagaaagatgaatacattttgttaaaaaaaaaaaaaaaa

SEQ ID NO: 120 - DNA, 567 bp, Homo sapiens
cagcgggcacgtgacatggccccggggagccgaggtgagcgtccagcttccggagccggagggggcccggcgtacccagcccccagcccgacgtgaccatgctgtcccgcctcctaaaagaacaccaggccaagcagaatgaacgcaaggagctgcaggaaaagaggaggcgagaggctatcactgcagcgacctgcctgacagaagctttggtggatcacctcaatgtgggtgtggcccaggcctacatgaaccagagaaagctggaccatgaggtgaagaccctacaggtccaggctgcccaatttgccaagcagacaggccagtggatcggaatggtggagaacttcaaccaggcactcaaggaaattggggatgtggagaactgggctcggagcatcgagctggacatgcgcaccattgccactgcactggaatatgtctacaaagggcagctgcagtctgccccttcctagcccctgttccctccccaaaccctatccctcctacctcacccgcagggggaaggagggaggctgacaagccttgaataaaacacaagcctcc

SEQ ID NO: 121 - DNA, 3074 bp, Homo sapiens
ccgctctccgctgcgggggaggccatggcggaaccttcccaggccccgaccccggccccggctgcgcagccccggccccttcagtccccagcccctgccccaactccgactcctgcacccagcccggcttcagccccgattccgactcccaccccggcaccagcccctgccccagctgcagccccagccggcagcacagggactggggggcccggggtaggaagtgggggggccgggagcgggggggatccggctcggcctggcctgagccagcagcagcgcgccagtcagaggaaggcgcaagtccgggggctgccccgcgccaagaagcttgagaagctaggggtcttctcggcttgcaaggccaatggaacctgtaagtgtaatggctggaaaaaccccaagccccccactgcaccccgcatagatctgcagcagccagctgccaacctgagtgagctgtgccgcagttgtgagcaccccttggctgaccacgtatcccacttggagaatgtgtcagaggatgagataaaccgactgctggggatggtggtggatgtggagaatctcttcatgtctgttcacaaggaagaggacacagacaccaagcaggtctatttctacctcttcaagctactgcggaaatgcatcctgcagatgacccggcctgtggtggaggggtccctgggcagccctccatttgagaaacctaatattgagcagggtgtgctgaactttgtgcagtacaagtttagtcacctggctccccgggagcggcagacgatgttcgagctctcaaagatgttcttgctctgccttaactactgggagcttgagacacctgcccagtttcggcagaggtctcaggctgaggacgtggctacctacaaggtcaattacaccagatggctctgttactgccacgtgccccagagctgtgatagcctcccccgctacgaaaccactcatgtctttgggcgaagccttctccggtccattttcaccgttacccgccggcagctgctggaaaagttccgagtggagaaggacaaattggtgcccgagaagaggaccctcatcctcactcacttccccaaattcctgtccatgctggaggaggagatctatggggcaaactctccaatctgggagtcaggcttcaccatgccaccctcagaggggacacagctggttccccggccagcttcagtcagtgcagcggttgttcccagcacccccatcttcagccccagcatgggtgggggcagcaacagctccctgagtctggattctgcaggggccgagcctatgccaggcgagaagaggacgctcccagagaacctgaccctggaggatgccaagcggctccgtgtgatgggtgacatccccatggagctggtcaatgaggtcatgctgaccatcactgaccctgctgccatgctggggcctgagacgagcctgctttcggccaatgcggcccgggatgagacagcccgcctggaggagcgccgcggcatcatcgagttccatgtcatcggcaactcactgacgcccaaggccaaccggcgggtgttgctgtggctcgtggggctgcagaatgtcttttcccaccagctgccgcgcatgcctaaggagtatatcgcccgcctcgtctttgacccgaagcacaagactctggccttgatcaaggatgggcgggtcatcggtggcatctgcttccgcatgtttcccacccagggcttcacggagattgtcttctgtgctgtcacctcgaatgagcaggtcaagggttatgggacccacctgatgaaccacctgaaggagtatcacatcaagcacaacattctctacttcctcacctacgccgacgagtacgccatcggctacttcaaaaagcagggtttctccaaggacatcaaggtgcccaagagccgctacctgggctacatcaaggactacgagggagcgacgctgatggagtgtgagctgaatccccgcatcccctacacggagctgtcccacatcatcaagaagcagaaagagatcatcaagaagctgattgagcgcaaacaggcccagatccgcaaggtctacccggggctcagctgcttcaaggagggcgtgaggcagatccctgtggagagcgttcctggcattcgagagacaggctggaagccattggggaaggagaaggggaaggagctgaaggaccccgaccagctctacacaaccctcaaaaacctgctggcccaaatcaagtctcaccccagtgcctggcccttcatggagcctgtgaagaagtcggaggcccctgactactacgaggtcatccgcttccccattgacctgaagaccatgactgagcggctgcgaagccgctactacgtgacccggaagctctttgtggccgacctgcagcgggtcatcgccaactgtcgcgagtacaaccccccggacagcgagtactgccgctgtgccagcgccctggagaagttcttctacttcaagctcaaggagggaggcctcattgacaagtaggcccatctttgggccgcagccctgacctggaatgtctccacctcggattctgatctgatccttagggggtgccctggccccacggacccgactcagcttgagacactccagccaagggtcctccggacccgatcctgcagctctttctggaccttcaggcacccccaagcgtgcagctctgtcccagccttcactgtgtgtgagaggtctcctgggttggggcccagcccctctagagtagctggtggccagggatgaaccttgcccagccgtggtggcccccaggcctggtccccaagagctttggaggcttggattcctgggcctggcccaggtggctgtttccctgaggaccagaactgctcattttagcttgagtgatggcttcaggggttggaagttcagcccaaactgaagggggccatgccttgtccagcactgttctgtcagtctcccccaggggtggggggtatggggaccattcattccctggcattaatcccttagagggaataataaagctttttatttctctg

SEQ ID NO: 122 - DNA, 3014 bp, Homo sapiens
ggggccgcgtcgacgcggaaaagaggccgtggggggcctcccagcgctggcagacaccgtgaggctggcagccgccggcacgcacacctagtccgcagtcccgaggaacatgtccgcagccagggcgcggagcagagtcccgggcaggagaaccaagggagggcgtgtgctgtggcggcggcggcagcggcagcggagccgctagtcccctccctcctgggggagcagctgccgccgctgccgccgccgccaccaccatcagcgcgcggggcccggccagagcgagccgggcgagcggcgcgctagggggagggcgggggcggggaggggggtgggcgaagggggcgggagggcgtggggggagggtctcgctctcccgactaccagagcccgagggagaccctggcggcggcggcggcgcctgacactcggcgcctcctgccgtgctccggggcggcatgtccgaggctggcggggccgggccgggcggctgcggggcaggagccggggcaggggccgggcccggggcgctgcccccgcagcctgcggcgcttccgcccgcgcccccgcagggctccccctgcgccgctgccgccgggggctcgggcgcctgcggtccggcgacggcagtggctgcagcgggcacggccgaaggaccgggaggcggtggctcggcccgaatcgccgtgaagaaagcgcaactacgctccgctccgcgggccaagaaactggagaaactcggagtgtactccgcctgcaaggccgaggagtcttgtaaatgtaatggctggaaaaaccctaacccctcacccactccccccagagccgacctgcagcaaataattgtcagtctaacagaatcctgtcggagttgtagccatgccctagctgctcatgtttcccacctggagaatgtgtcagaggaagaaatgaacagactcctgggaatagtattggatgtggaatatctctttacctgtgtccacaaggaagaagatgcagataccaaacaagtttatttctatctatttaagctcttgagaaagtctattttacaaagaggaaaacctgtggttgaaggctctttggaaaagaaacccccatttgaaaaacctagcattgaacagggtgtgaataactttgtgcagtacaaatttagtcacctgccagcaaaagaaaggcaaacaatagttgagttggcaaaaatgttcctaaaccgcatcaactattggcatctggaggcaccatctcaacgaagactgcgatctcccaatgatgatatttctggatacaaagagaactacacaaggtggctgtgttactgcaacgtgccacagttctgcgacagtctacctcggtacgaaaccacacaggtgtttgggagaacattgcttcgctcggtcttcactgttatgaggcgacaactcctggaacaagcaagacaggaaaaagataaactgcctcttgaaaaacgaactctaatcctcactcatttcccaaaatttctgtccatgctagaagaagaagtatatagtcaaaactctcccatctgggatcaggattttctctcagcctcttccagaaccagccagctaggcatccaaacagttatcaatccacctcctgtggctgggacaatttcatacaattcaacctcatcttcccttgagcagccaaacgcagggagcagcagtcctgcctgcaaagcctcttctggacttgaggcaaacccaggagaaaagaggaaaatgactgattctcatgttctggaggaggccaagaaaccccgagttatgggggatattccgatggaattaatcaacgaggttatgtctaccatcacggaccctgcagcaatgcttggaccagagaccaattttctgtcagcacactcggccagggatgaggcggcaaggttggaagagcgcaggggtgtaattgaatttcacgtggttggcaattccctcaaccagaaaccaaacaagaagatcctgatgtggctggttggcctacagaacgttttctcccaccagctgccccgaatgccaaaagaatacatcacacggctcgtctttgacccgaaacacaaaacccttgctttaattaaagatggccgtgttattggtggtatctgtttccgtatgttcccatctcaaggattcacagagattgtcttctgtgctgtaacctcaaatgagcaagtcaagggctatggaacacacctgatgaatcatttgaaagaatatcacataaagcatgacatcctgaacttcctcacatatgcagatgaatatgcaattggatactttaagaaacagggtttctccaaagaaattaaaatacctaaaaccaaatatgttggctatatcaaggattatgaaggagccactttaatgggatgtgagctaaatccacggatcccgtacacagaattttctgtcatcattaaaaagcagaaggagataattaaaaaactgattgaaagaaaacaggcacaaattcgaaaagtttaccctggactttcatgttttaaagatggagttcgacagattcctatagaaagcattcctggaattagagagacaggctggaaaccgagtggaaaagagaaaagtaaagagcccagagaccctgaccagctttacagcacgctcaagagcatcctccagcaggtgaagagccatcaaagcgcttggcccttcatggaacctgtgaagagaacagaagctccaggatattatgaagttataaggttccccatggatctgaaaaccatgagtgaacgcctcaagaataggtactacgtgtctaagaaattattcatggcagacttacagcgagtctttaccaattgcaaagagtacaacgccgctgagagtgaatactacaaatgtgccaatatcctggagaaattcttcttcagtaaaattaaggaagctggattaattgacaagtgattttttttccccctctgcttcttagaaactcaccaagcagtgtgcctaaagcaaggt

SEQ ID NO: 123 - DNA, 7869 bp, Homo sapiens
ggcacgaggtttggggcatctccgcggtccggcccggggccccgggatctcggctgtccttcctcccggcataagatgcacatttttctgctctggagccgggaatgaaatattcttgagttcttacaactttatgacgagacccatgtgtggtgctattgagaaattcattgggaagttggaagacatttcaaacaacaggttgttttggtttctatagtacaattggggtggcattctgttttgtgaaaggaggaaggacttaggccagaaaactcatatgctatggttaactggttcccagcctccgagaatcttgttttccatggtgtaaaacttactcagcatcaggataagggataacgactctatggatatacagaatccttcaccatggtaaaactcgcaaacccgctttatactgagtggattttggaggccatcaaaaaagtgaaaaagcagaaacagcgtccttcagaagaaaggatatgcaatgctgtgtcttcatcccatggcttggatcgtaaaactgttttagaacaattggagttgagtgttaaagatggaacaattttaaaagtctcaaataaaggactcaattcctataaagatcctgataatcctgggcgaatagcacttcctaagcctcggaaccatggaaaattggataataaacaaaatgtggattggaataaactgataaagcgggcagttgagggcttggcagagtctggtggctcaactttgaaaagcattgaacgttttttgaaaggtcagaaggatgtgtctgcattattcggaggcagtgctgcctctggctttcaccagcagttacgattggctatcaaacgtgccattggccacggcagactccttaaagatggacctctttatcggctcaacactaaagcaaccaacgtggatgggaaagagagttgtgagtctctttcctgtttacctccagtgtcccttcttccacatgaaaaggataagccggttgctgaaccaatccccatctgtagtttctgtcttggtacaaaagaacaaaaccgagaaaagaagccagaggaactcatctcctgtgccgactgtggcaacagtggccatccatcctgtttaaagttttcccctgaactaacggttcgagtgaaggccttacggtggcagtgcatcgagtgtaaaacatgcagctcctgtcgagatcaaggcaaaaatgcggataacatgctcttttgtgattcatgtgaccgaggttttcacatggagtgttgtgatccgccactcacccgtatgccaaaaggcatgtggatatgtcaaatatgtcgacctaggaaaaaaggacgaaaacttctacaaaagaaggcagcacagataaaacggcgctatactaatccaataggacgtccaaaaaacaggttaaagaaacaaaacacggtatcaaaaggtcccttcagcaaagttcgaactggccctggaaggggtaggaaacgaaaaatcactctttccagccaatcagcatcatcatcatcagaagaaggatatttagagcggatagatggcttggacttctgcagagatagcaatgtctccttgaggttcaacaagaaaaccaaagggctcattgatggccttaccaaattttttaccccttcccctgatgggcggaaagctcggggggaagtggtggactactctgagcaatatcgaatcagaaagaggggcaacaggaaatcaagcacttcagattggcccacagacaatcaggatggctgggatggcaaacaagaaaatgaggagcgactttttgggagccaggaaatcatgactgagaaagatatggaattatttcgtgatatccaagaacaagcactgcagaaagttggagtgactggtccccctgatccacaagtccgctgtccctctgtcattgagtttgggaagtatgaaattcacacctggtactcctccccatatcctcaagaatactcaaggctgcccaaattgtatctttgtgaattttgtctaaaatatatgaaaagtagaactattctgcagcagcacatgaagaaatgtggttggttccatcctcctgccaatgagatttacagaaagaataatatttctgtctttgaggttgatgggaatgtgagtaccatttattgtcaaaacctgtgtcttttggcaaagttgtttcttgaccacaaaaccctctattacgatgtggagccatttcttttttatgtactaacacagaatgatgtcaagggctgccaccttgttggctacttttctaaggaaaagcactgccaacagaagtacaatgtttcctgtataatgattcttcctcaataccagcgtaagggctatggcaggtttctcatcgatttcagttatttgttatcaaagcgtgaaggccaagcagggtctccagagaaaccgttatctgatctgggtcgtctttcctacatggcatattggaaaagtgtaatattggagtgcctttatcaccaaaatgacaagcagatcagcattaagaagttaagcaagttgactggaatctgccctcaagacatcacttccacactccaccacctacgaatgctggacttccgtagtgaccaatttgtgattatccgccgggaaaaacttatccaggatcacatggcaaagcttcagctgaatttgcgacctgtagatgtagatccagaatgtttgcgctggactccagtcatagtgtccaactctgtggtctcagaggaggaagaagaggaggctgaggaaggagaaaacgaagagccacagtgccaggaaagagaattagagatcagtgtgggaaagtctgtgtctcatgagaacaaagaacaagattcttattcagtagaaagtgaaaagaaaccagaagttatggctccagtcagttctacacgtttgagcaaacaagtccttcctcatgatagtcttcctgcaaatagccagccatctcggaggggccgctgggggaggaagaacagaaaaacccaggaacgttttggtgataaagattctaaactgctcttggaagagacgtcttcagctcctcaggaacaatatggagaatgtggggagaaatcagaagccacccaggaacaatacactgaaagtgaagaacagctggtggcttctgaggagcagccaagccaggacgggaaacctgaccttcccaagagaagactcagtgagggggttgagccctggcgaggacagctcaagaaaagccctgaggctctgaagtgcagattaacagaaggaagtgagaggctgccccgtcgctacagtgagggtgacagggctgtcctcaggggcttcagtgagagcagcgaggaggaggaggagccggaaagccctcggtcaagctcgccaccaattctcacaaagcccacgctgaagcgaaagaaaccatttctccaccgaaggaggagagtccgaaagcgcaaacaccacaatagcagtgtagtcacagaaactatttctgagaccactgaagtgttagatgaaccttttgaagattctgactccgagaggccaatgccaagattagaacccacatttgagatcgatgaagaagaggaggaagaggatgaaaatgaacttttccctagagaatacttccgtcgtttgtcttcgcaggatgtactcaggtgtcagtcctcttctaagaggaagtctaaagatgaagaagaagatgaagagtcagatgatgctgatgacactcctatcttaaagccagtatctcttttgcgaaaacgtgatgtgaagaattctcctcttgagccagatacatccacacctttgaaaaagaaaaagggatggcccaaaggcaagagccgcaaaccaatccactggaagaaaagacctggtcgaaaaccaggatttaagttgagtcgggaaatcatgccagtttctactcaagcatgcgtcattgagcccatcgtttccattcctaaagctggacgtaaacccaagatccaggagagtgaagaaactgttgagccaaaagaagacatgcccctacccgaggagaggaaggaggaggaggagatgcaagcagaggcagaagaggctgaagagggtgaggaagaggatgcagccagcagtgaagtcccagcagcctctccagcagacagcagcaatagtcctgagaccgaaaccaaggagcctgaggtggaggaggaagaagagaagccccgtgtctcagaggagcagaggcagtcagaggaggagcagcaggaattagaggagccagagccagaggaggaggaagatgcagctgcagagactgcccagaatgacgaccacgacgctgatgatgaggatgatggccacctggagtccacaaagaaaaaggagctagaggaacagcccacgagggaagatgtcaaggaggagcctggtgttcaagagtcttttttagatgctaatatgcagaagagtagggaaaagataaaggataaagaggaaaccgagctggattccgaagaggagcagccttcccatgacacgtccgtggtgtcagagcagatggctgggtctgaggacgaccacgaagaagactcccacactaaggaagagttaatcgaattaaaagaggaggaagagattcctcatagtgagctggatctggaaactgtacaggcagtgcagtctttgactcaagaagaaagcagtgagcatgagggcgcctaccaggactgtgaggaaactcttgcggcgtgtcagaccctgcagagttacacccaggctgacgaggaccctcagatgtccatggttgaagactgtcatgcgtcagaacataatagccctatctcctccgttcagtctcaccccagccagtcagtccgttcggtcagcagtcccaacgtgcctgcccttgagagtggctacacccagatcagcccagaacaaggatccctgtccgcaccctctatgcagaacatggagaccagccccatgatggatgtgccttccgtatcagaccactctcagcaggtggtggacagcggcttcagtgacctgggcagcattgagagcaccactgaaaactatgagaacccaagcagttacgactccacgatgggcggcagcatctgtgggaacagctcttcccagagcagctgctcctacggtgggctgtcgtcctccagcagcctcacccagagcagctgtgtggtcactcagcagatggccagcatgggcagcagctgcagcatgatgcagcagagcagcgtccagcctgctgccaactgcagcatcaagtcacctcagagctgcgtggtggagaggcctcccagtaaccagcagcagcagccgccaccaccgcctccacagcagccacagccgccgccgccacaaccacaaccagcaccacagcctccaccaccccagcagcagccgcaacagcagccgcagcctcagccccagcagcctccacccccaccccctccccagcagcagcccccgctgtcacagtgtagtatgaataacagtttcaccccagctcctatgatcatggagataccagaatctggaagcactgggaacataagtatctatgagaggattccaggggattttggtgccggcagctactctcaaccatcagccaccttcagcctagccaagctgcagcagctgaccaacaccattatggaccctcatgccatgccttatagccattctcctgctgtgacttcctatgcaaccagtgtttctctgtccaatacaggactggctcagctggctccatctcatcccttagctgggactcctcaagcacaagccaccatgacgccacccccaaacttggcatccactaccatgaacctcacatctcctctgcttcagtgcaacatgtctgccaccaacattggcattcctcacacgcagagattgcaagggcaaatgccagtgaaggggcacatttccatccgctccaagtctgcgccactgccctctgcggctgctcaccagcagcagctgtatggccgtagcccatcggcagttgccatgcaggctggccctcgcgcactggctgttcagcgtggcatgaacatgggggttaatctgatgcctactcccgcctataatgtcaattccatgaatatgaacaccttgaatgccatgaacagctatcgaatgacacagcccatgatgaacagcagttaccatagtaaccctgcctacatgaaccagacagcacagtatcctatgcagatgcagatgggaatgatggggagccaggcctatacccagcagcctatgcagcctaaccctcatgggaacatgatgtacacaggcccctcccatcacagctacatgaacgctgctggcgtgcccaagcagtcactcaacggaccttacatgagaagatgagcaagatgaacttgcaatcaaaaacttaaatatatataaataaaggaaccttttatactgacaaaccagagaaaaatggacctttttccagttaaaatattgctgtagatttagaggaatttttctttggtttattttattttttagaaaacctgatcttctcttttttttgggttcattttgttgtgggttttggttttcttcacaatcttgaacattttacagtagaactcatctaaaaatggatttggggatggggaaacatgcacaaaatcttttcataattaaaaagagccttactttctttacataccacatggacagaatttgtgtaaaagtgaattatctttattttaaaatgtatgtttcccctcactgtttgcagctcccaatgttgtcatttttaaatgttatatacatctcaagggttaaccagaccctttcctccaaacccaacctttcatttcctacttcattccagcaggaggcacttaggggagactcggatggggacatggagaacaacccaagctccttaaacttattattattgttaatattattattattattattaataaagtgaggcaggaaaatgcttctccttttaaaatcccctccactcctcacacacacacacctcttgaaacccttccccaagaatgtttctttatagacggacttcattgaaatctttgttgttcttgaatcaagtgtaatataatttttttcttcttttttaaaatattcccactcagcactcagagacacaaaaatactgtaagtctcaattaacagcagaatctcagagaaaagctgtttgcaatccaaatccagcctttggaggaatagagatggtcaattaacaatcaaaaagaggagattaacctcttgtttttttaccacctggtgaatcagccataacgcacacacacgccacccagcctcttgtttctagtatgtactttgaaatgctaactgagggtcttgatgcttgagcctttgactgataaaactcaaatagcagtccccagtgatttgcctcttaggttctttcttaaattgttggtggatgactgtacattttagtgatttgaaaaataactgacaaaccattgaaacagtttattttatgttggaagagatggcgcagatgtgtgtcagaagggagatcacggtgtgagtttcgtagctatttaagtgatacatacctctagtttttgtatgtcttttgagatcctgagttcatcccctgtgaatcagagtgcacaagcacctctcctgtgagtggctaatgagaagagggacagaccgaccaccagcacagtagggcagatctggacagcagaatgttataacgcaagttcatgtgttgctcccaactccattctcttttctctcgtgcaaccagtttgcccattctcttcctattacttgctccagggataggtaaaaaaaaaa

SEQ ID NO: 124 - DNA, 3504 bp, Homo sapiens
gccgctgcccgaatcggaaccgtcgggccgcagccgccggcaatgccgcgaaggaagaggaatgcaggcagtagttcagatggaaccgaagattccgatttttctacagatctcgagcacacagacagttcagaaagtgatggcacatcccgacgatctgctcgagtcacccgctcctcagccaggctaagccagagttctcaagattccagtcctgttcgaaatctgcagtcttttggcactgaggagcctgcttactctaccagaagagtgacccgtagtcagcagcagcctaccccagtgacaccgaaaaaataccctcttcggcagactcgttcatctggttcagaaactgagcaagtggttgatttttcagatagagaaactaaaaatacagctgatcatgatgagtcaccgcctcgaactccaactggaaatgcgccttcttctgagtctgacatagatatctccagccccaatgtatctcacgatgagagcattgccaaggacatgtccctgaaggactcaggcagtgatctctctcatcgccccaagcgccgtcgcttccatgaaagctacaacttcaatatgaagtgtcctacaccaggctgtaactctctaggacaccttacaggaaaacatgagagacatttctccatctcaggatgcccactgtatcataacctctcagctgacgaatgcaaggtgagagcacagagccgggataagcagatagaagaaaggatgctgtctcacaggcaagatgacaacaacaggcatgcaaccaggcaccaggcaccaacggagaggcagcttcgatataaggaaaaagtggctgaactcaggaagaaaagaaattctggactgagcaaagaacagaaagagaaatatatggaacacagacagacctatgggaacacacgggaacctcttttagaaaacctgacaagcgagtatgacttggatcttttccgaagagcacaagcccgggcttcagaggatttggagaagttaaggctgcaaggccaaatcacagagggaagcaacatgattaaaacaattgcttttggccgctatgagcttgatacctggtatcattctccatatcctgaagaatatgcacggctgggacgtctctatatgtgtgaattctgtttaaaatatatgaagagccaaacgatactccgccggcacatggccaaatgtgtgtggaaacacccacctggtgatgagatatatcgcaaaggttcaatctctgtgtttgaagtggatggcaagaaaaacaagatctactgccaaaacctgtgcctgttggccaaactttttctggaccacaagacattatattatgatgtggagcccttcctgttctatgttatgacagaggcggacaacactggctgtcacctgattggatatttttctaaggaaaagaattcattcctcaactacaacgtctcctgtatccttactatgcctcagtacatgagacagggctatggcaagatgcttattgatttcagttatttgctttccaaagtcgaagaaaaagttggctccccagaacgtccactctcagatctggggcttataagctatcgcagttactggaaagaagtacttctccgctacctgcataattttcaaggcaaagagatttctatcaaagaaatcagtcaggagacggctgtgaatcctgtggacattgtcagcactctgcaagcccttcagatgctcaaatactggaagggaaaacacctagttttaaagagacaggacctgattgatgagtggatagccaaagaggccaaaaggtccaactccaataaaaccatggatcccagctgcttaaaatggacccctcccaagggcacttaaagtgacctgtcattccgagccagcgaaccccagcagtaggaatccgtaccctagggatctgtctgtcatttctctgttgctcttgtgattggcaagtacagtatcctttgggaaggccatccccctcaggactgtcctggctccgacctttgtgtacactgcagacgctggttctgaggaactgttgtttcggcctcagtgaggttgcctggatgggatctgtattagacttgagtgcaggtctctcagcactgacccaaggagttctgttatggtactgtacctgtccagtcactggttctctcctcatgtcctctcgccccatgaggttgtgttgtgtcttctaagcgtggtactagtgcttgccacctggtcaccagacctccaaatatggctgccaccaccaggacctttccagttactccttatatgtgtgttctatggaggggcagggaaaaggtggcacttgtgagtgtgtgtggattggcagggggtccattcactttgggttccatcttgctttaaatttcttcattttgattaagagacctctttttgatctgtattgggctaaccagagccaaatacttttgaagagtttcccagggactagtcatggtaatagcatataattgatctgaatgagatggagagaagaatgaaggggtggtggttctgggtttgatttgagttcacctgtgggcagtgggcagtgggcagtgtcttggtgaaagggaacggatactactttttgcctcaccgtaaagtactcactagtaaatatttccttctctctttactcccactttttacgtttgcaggtgccaaagtaatgtccacttttccctttcatgctgcatattaactggttaattatactgcagaaaccttttcacctccactagtctgatacagtacatctgtacttccatataccttgcactgattttgtctgagtgccctgggagaagtagaaaatgattgaaagtgacttccgtatctcagcccatgactcagcaaggcagaatggccacccctgccaaagtttgcttctcttttcaacagtgcctcaccctccctctaggattaaagtgcttctgcccttccacgaactcctcctccatttcctttttgggatttgtcaccatccttctattctctggtcttctatttttggtgttgttcaagtgaaggaagagatgttccctctaatttctctctagcccattataacctgctatcttggggcaacttttgatgtatgacatgtcacccttcccaacttggtctcctccaacatgctgtcttcatgtggagccctcaccacaatccctgactccggtcatttgtgcctttctcttgtcatctctgtacactacttatattcactgtgggttgggggagctaattttaagcatgttcagtggcagctcccctccagtttcagtgtcactgttaaaatttatcaaaaagcaacttcactaggggttttcttaagggataaaggccttttacagaagctaaacccttccccacatgtggtagaatgtgctcttctatatctactcctcaataaagcatgttctctgctcaaaaaaaaaaaaaaaaaaaaaaaaaaaaaaaaaa

SEQ ID NO: 125 - DNA, 3423 bp, Homo sapiens
atatcatcgacagggagcacagtgggctttctcctcaagatgacactaattctggaatgtcaattccccgagtaaatccctcggtcaatcctagtatctctccagctcatggtgtggctcgttcatccacattgccaccatccaacagcaacatggtatccaccagaataaaccgccagcagagctcagaccttcatagcagcagtcatagtaattctagcaacagccaaggaagtttcggatgctcacccggaagtcagattgtagccaatgttgccttaaacaaaggacaggccagttcacagagcagtaaaccctctttaaacctcaataatcctcctatggaaggtacaggaatatccctagcacagttcatgtctccaaggagacaggttacttctggattggcaacaaggcccaggatgccaaacaattcctttcctcctaatatttcgacattaagctctcccgttggcatgacaagtagtgcctgtaataataataaccgatcttattcaaacatcccagtaacatctttacagggtatgaatgaaggacccaataactccgttggcttctctgccagttctccagtcctcaggcagatgagctcacagaattcacctagcagattaaatatacaaccagcaaaagctgagtccaaagataacaaagagattgcctcaactttaaatgaaatgattcaatctgacaacagctctagtgatggcaaacctctggattcagggcttctgcataacaatgacagactttcagatggagacagtaaatactctcaaaccagtcacaaactagtgcagcttttgacaacaactgccgaacagcagttacggcatgctgatatagacacaagctgcaaagatgtcctgtcttgcacaggcacttccaactctgcctctgctaactcttcaggaggttcttgtccctcttctcatagctcattgacagcacggcataaaattctacaccggctcttacaggagggtagcccctcagatatcaccactttgtctgtcgagcctgataaaaaggacagtgcatctacttctgtgtcagtgactggacaggtacaaggaaactccagtataaaactagaactggatgcttcaaagaaaaaagaatcaaaagaccatcagctcctacgctatcttttagataaagatgagaaagatttaagatcaactccaaacctgagcctggatgatgtaaaggtgaaagtggaaaagaaagaacagatggatccatgtaatacaaacccaaccccaatgacgaaggccactcctgaggaaataaaactggaggcccagagccagtttacagctgaccttgaccagtttgatcagttactgcccacgctggagaaggcagcacagttgccaggcttatgtgagacagacaggatggatggtgcggtcaccagtgtaaccatcaaatcggagatcctgccagcttcacttcagtccgccactgccagacccacttccaggctgaatagattacctgagctggaattggaagcaattgataaccaatttggacaaccaggaacaggcgatcagattccatggacaaataatacagtgacagctataaatcagagtaaatcagaagaccagtgtattagctcacaattagatgagcttctctgtccacccacaacagtagaagggagaaatgatgagaaggctcttcttgaacagctggtatccttccttagtggcaaagatgaaactgagctagctgaactagacagagctctgggaattgacaaacttgttcaggggggtggattagatgtattatcagagagatttccaccacaacaagcaacgccacctttgatcatggaagaaagacccaacctttattcccagccttactcttctccttttcctactgccaatctccctagccctttccaaggcatggtcaggcaaaaaccttcactggggacgatgcctgttcaagtaacacctccccgaggtgctttttcacctggcatgggcatgcagcccaggcaaactctaaacagacctccggctgcacctaaccagcttcgacttcaactacagcagcgattacagggacaacagcagttgatacaccaaaatcggcaagctatcttaaaccagtttgcagcaactgctcctgttggcatcaatatgagatcaggcatgcaacagcaaattacacctcagccacccctgaatgctcaaatgttggcacaacgtcagcgggaactgtacagtcaacagcaccgacagaggcagctaatacagcagcaaagagccatgcttatgaggcagcaaagctttgggaacaacctccctccctcatctggactaccagttcaaacggggaacccccgtcttcctcagggtgctccacagcaattcccctatccaccaaactatggtacaaatccaggaaccccacctgcttctaccagcccgttttcacaactagcagcaaatcctgaagcatccttggccaaccgcaacagcatggtgagcagaggcatgacaggaaacataggaggacagtttggcactggaatcaatcctcagatgcagcagaatgtcttccagtatccaggagcaggaatggttccccaaggtgaggccaactttgctccatctctaagccctgggagctccatggtgccgatgccaatccctcctcctcagagttctctgctccagcaaactccacctgcctccgggtatcagtcaccagacatgaaggcctggcagcaaggagcgataggaaacaacaatgtgttcagtcaagctgtccagaaccagcccacgcctgcacagccaggagtatacaacaacatgagcatcaccgtttccatggcaggtggaaatacgaatgttcagaacatgaacccaatgatggcccagatgcagatgagctctttgcagatgccaggaatgaacactgtgtgccctgagcagataaatgatcccgcactgagacacacaggcctctactgcaaccagctctcatccactgaccttctcaaaacagaagcagatggaacccagcaggtgcaacaggttcaggtgtttgctgacgtccagtgtacagtgaatctggtaggcggggacccttacctgaaccagcctggtccactgggaactcaaaagcccacgtcaggaccacagaccccccaggcccagcagaagagcctccttcagcagctactgactgaataaccacttttaaaggaatgtgaaatttaaataatagacatacagagatatacaaatatattatatatttttctgagatttttgatatctcaatctgcagccattcttcaggtcgtagcatttggagcaaaaaaaaaaaaaaaaaatcgatgtcgactcgacgacactacggcgacagcaacgg

SEQ ID NO: 126 - DNA, 6754 bp, Homo sapiens
gtcgacgtggcggccggcggcggctgcgggctgagcggcgagtttccgatttaaagctgagctgcgaggaaaatggcggcgggaggatcaaaatacttgctggatggtggactcagagaccaataaaaataaactgcttgaacatcctttgactggttagccagttgctgatgtatattcaagatgagtggattaggagaaaacttggatccactggccagtgattcacgaaaacgcaaattgccatgtgatactccaggacaaggtcttacctgcagtggtgaaaaacggagacgggagcaggaaagtaaatatattgaagaattggctgagctgatatctgccaatcttagtgatattgacaatttcaatgtcaaaccagataaatgtgcgattttaaaggaaacagtaagacagatacgtcaaataaaagagcaaggaaaaactatttccaatgatgatgatgttcaaaaagccgatgtatcttctacagggcagggagttattgataaagactccttaggaccgcttttacttcaggcattggatggtttcctatttgtggtgaatcgagacggaaacattgtatttgtatcagaaaatgtcacacaatacctgcaatataagcaagaggacctggttaacacaagtgtttacaatatcttacatgaagaagacagaaaggattttcttaagaatttaccaaaatctacagttaatggagtttcctggacaaatgagacccaaagacaaaaaagccatacatttaattgccgtatgttgatgaaaacaccacatgatattctggaagacataaacgccagtcctgaaatgcgccagagatatgaaacaatgcagtgctttgccctgtctcagccacgagctatgatggaggaaggggaagatttgcaatcttgtatgatctgtgtggcacgccgcattactacaggagaaagaacatttccatcaaaccctgagagctttattaccagacatgatctttcaggaaaggttgtcaatatagatacaaattcactgagatcctccatgaggcctggctttgaagatataatccgaaggtgtattcagagattttttagtctaaatgatgggcagtcatggtcccagaaacgtcactatcaagaagttaccagtgatgggatattttccccaacagcttatcttaatggccatgcagaaaccccagtatatcgattctcgttggctgatggaactatagtgactgcacagacaaaaagcaaactcttccgaaatcctgtaacaaatgatcgacatggctttgtctcaacccacttccttcagagagaacagaatggatatagaccaaacccaaatcctgttggacaagggattagaccacctatggctggatgcaacagttcggtaggcggcatgagtatgtcgccaaaccaaggcttacagatgccgagcagcagggcctatggcttggcagaccctagcaccacagggcagatgagtggagctaggtatgggggttccagtaacatagcttcattgacccctgggccaggcatgcaatcaccatcttcctaccagaacaacaactataggctcaacatgagtagccccccacatgggagtcctggtcttgccccaaaccagcagaatatcatgatttctcctcgtaatcgtgggagtccaaagatagcctcacatcagttttctcctgttgcaggtgtgcactctcccatggcatcttctggcaatactgggaaccacagcttttccagcagctctctcagtgccctgcaagccatcagtgaaggtgtggggacttcccttttatctactctgtcatcaccaggccccaaattggataactctcccaatatgaatattacccaaccaagtaaagtaagcaatcaggattccaagagtcctctgggcttttattgcgaccaaaatccagtggagagttcaatgtgtcagtcaaatagcagagatcacctcagtgacaaagaaagtaaggagagcagtgttgagggggcagagaatcaaaggggtcctttggaaagcaaaggtcataaaaaattactgcagttacttacctgttcttctgatgaccggggtcattcctccttgaccaactcccccctagattcaagttgtaaagaatcttctgttagtgtcaccagcccctctggagtctcctcctctacatctggaggagtatcctctacatccaatatgcatgggtcactgttacaagagaagcaccggattttgcacaagttgctgcagaatgggaattcaccagctgaggtagccaagattactgcagaagccactgggaaagacaccagcagtataacttcttgtggggacggaaatgttgtcaagcaggagcagctaagtcctaagaagaaggagaataatgcacttcttagatacctgctggacagggatgatcctagtgatgcactctctaaagaactacagccccaagtggaaggagtggataataaaatgagtcagtgcaccagctccaccattcctagctcaagtcaagagaaagaccctaaaattaagacagagacaagtgaagagggatctggagacttggataatctagatgctattcttggtgatctgactagttctgacttttacaataattccatatcctcaaatggtagtcatctggggactaagcaacaggtgtttcaaggaactaattctctgggtttgaaaagttcacagtctgtgcagtctattcgtcctccatataaccgagcagtgtctctggatagccctgtttctgttggctcaagtcctccagtaaaaaatatcagtgctttccccatgttaccaaagcaacccatgttgggtgggaatccaagaatgatggatagtcaggaaaattatggctcaagtatgggagactggggcttaccaaactcaaaggccggcagaatggaacctatgaattcaaactccatgggaagaccaggaggagattataatacttctttacccagacctgcactgggtggctctattcccacattgcctcttcggtctaatagcataccaggtgcgagaccagtattgcaacagcagcagcagatgcttcaaatgaggcctggtgaaatccccatgggaatgggggctaatccctatggccaagcagcagcatctaaccaactgggttcctggcccgatggcatgttgtccatggaacaagtttctcatggcactcaaaataggcctcttcttaggaattccctggatgatcttgttgggccaccttccaacctggaaggccagagtgacgaaagagcattattggaccagctgcacactcttctcagcaacacagatgccacaggcctggaagaaattgacagagctttgggcattcctgaacttgtcaatcagggacaggcattagagcccaaacaggatgctttccaaggccaagaagcagcagtaatgatggatcagaaggcaggattatatggacagacatacccagcacaggggcctccaatgcaaggaggctttcatcttcagggacaatcaccatcttttaactctatgatgaatcagatgaaccagcaaggcaattttcctctccaaggaatgcacccacgagccaacatcatgagaccccggacaaacacccccaagcaacttagaatgcagcttcagcagaggctgcagggccagcagtttttgaatcagagccgacaggcacttgaattgaaaatggaaaaccctactgctggtggtgctgcggtgatgaggcctatgatgcagccccagcagggttttcttaatgctcaaatggtcgcccaacgcagcagagagctgctaagtcatcacttccgacaacagagggtggctatgatgatgcagcagcagcaacagcagcagcagcagcagcagcagcagcaacagcaacagcaacagcaacagcagcaacagcagcaaacccaggccttcagcccacctcctaatgtgactgcttcccccagcatggatgggcttttggcaggacccacaatgccacaagctcctccgcaacagtttccatatcaaccaaattatggaatgggacaacaaccagatccagcctttggtcgagtgtctagtcctcccaatgcaatgatgtcgtcaagaatgggtccctcccagaatcccatgatgcaacacccgcaggctgcatccatctatcagtcctcagaaatgaagggctggccatcaggaaatttggccaggaacagctccttttcccagcagcagtttgcccaccaggggaatcctgcagtgtatagtatggtgcacatgaatggcagcagtggtcacatgggacagatgaacatgaaccccatgcccatgtctggcatgcctatgggtcctgatcagaaatactgctgacatctctgcaccaggacctcttaaggaaaccactgtacaaatgacactgcactaggattattgggaaggaatcattgttccaggcatccatcttggaagaaaggaccagctttgagctccatcaagggtattttaagtgatgtcatttgagcaggactggattttaagccgaagggcaatatctacgtgtttttcccccctccttctgctgtgtatcatggtgttcaaaacagaaatgttttttggcattccacctcctagggatataattctggagacatggagtgttactgatcataaaacttttgtgtcacttttttctgccttgctagccaaaatctcttaaatacacgtaggtgggccagagaacattggaagaatcaagagagattagaatatctggtttctctagttgcagtattggacaaagagcatagtcccagccttcaggtgtagtagttctgtgttgaccctttgtccagtggaattggtgattctgaattgtcctttactaatggtgttgagttgctctgtccctattatttgccctaggctttctcctaatgaaggttttcatttgccattcatgtcctgtaatacttcacctccaggaactgtcatggatgtccaaatggctttgcagaaaggaaatgagatgacagtatttaatcgcagcagtagcaaacttttcacatgctaatgtgcagctgagtgcactttatttaaaaagaatggataaatgcaatattcttgaggtcttgagggaatagtgaaacacattcctggtttttgcctacacttacgtgttagacaagaactatgatttttttttttaaagtactggtgtcaccctttgcctatatggtagagcaataatgctttttaaaaataaacttctgaaaacccaaggccaggtactgcattctgaatcagaatctcgcagtgtttctgtgaatagatttttttgtaaatatgacctttaagatattgtattatgtaaaatatgtatatacctttttttgtaggtcacaacaactcatttttacagagtttgtgaagctaaatatttaacattgttgatttcagtaagctgtgtggtgaggctaccagtggaagagacatcccttgacttttgtggcctgggggaggggtagtgcaccacagcttttccttccccaccccccagccttagatgcctcgctcttttcaatctcttaatctaaatgctttttaaagagattatttgtttagatgtaggcattttaattttttaaaaattcctctaccagaactaagcactttgttaatttggggggaaagaatagatatggggaaataaacttaaaaaaaaatcaggaatttaaaaaaaacgagcaatttgaagagaatcttttggattttaagcagtccgaaataatagcaattcatgggctgtgtgtgtgtgtgtatgtgtgtgtgtgtgtgtgtatgtttaattatgttaccttttcatcccctttaggagcgttttcagattttggttcgtaagacctgaatcccatattgagatctcgagtagaatccttggtgtggtttctggtgtctgctcagctgtcccctcattctactaatgtgatgctttcattatgtccctgtggattagaatagtgtcagttatttcttaagtaactcagtacccagaacagccagttttactgtgattcagagccacagtctaactgagcaccttttaaacccctccctcttctgccccctaccacttttctgctgttgcctctctttgacacctgttttagtcagttgggaggaagggaaaaatcaagtttaattccctttatctgggttaattcatttggttcaaatagttgacggaattgggtttctgaatgtctgtgaatttcagaggtctctgctagccttggtatcattttctagcaataactgagagccagttaattttaagaatttcacacatttagccaatctttctagatgtctctgaaggtaagatcatttaatatctttgatatgcttacgagtaagtgaatcctgattatttccagacccaccaccagagtggatcttattttcaaagcagtatagacaattatgagtttgccctctttcccctaccaagttcaaaatatatctaagaaagattgtaaatccgaaaacttccattgtagtggcctgtgcttttcagatagtatactctcctgtttggagacagaggaagaaccaggtcagtctgtctctttttcagctcaattgtatctgacccttctttaagttatgtgtgtggggagaaatagaatggtgctcttatgtcgac

SEQ ID NO: 127 - DNA, 6156 bp, Homo sapiens
ggcggccgcagcctcggctacagcttcggcggcgaaggtcagcgccgacggcagccggcacctgacggcgtgaccgacccgagccgatttctcttggatttggctacacacttatagatcttctgcactgtttacaggcacagttgctgatatgtgttcaagatgagtgggatgggagaaaatacctctgacccctccagggcagagacaagaaagcgcaaggaatgtcctgaccaacttggacccagccccaaaaggaacactgaaaaacgtaatcgtgaacaggaaaataaatatatagaagaacttgcagagttgatttttgcaaattttaatgatatagacaactttaacttcaaacctgacaaatgtgcaatcttaaaagaaactgtgaagcaaattcgtcagatcaaagaacaagagaaagcagcagctgccaacatagatgaagtgcagaagtcagatgtatcctctacagggcagggtgtcatcgacaaggatgcgctggggcctatgatgcttgaggcccttgatgggttcttctttgtagtgaacctggaaggcaacgttgtgtttgtgtcagagaatgtgacacagtatctaaggtataaccaagaagagctgatgaacaaaagtgtatatagcatcttgcatgttggggaccacacggaatttgtcaaaaacctgctgccaaagtctatagtaaatgggggatcttggtctggcgaacctccgaggcggaacagccataccttcaattgtcggatgctggtaaaacctttacctgattcagaagaggagggtcatgataaccaggaagctcatcagaaatatgaaactatgcagtgcttcgctgtctctcaaccaaagtccatcaaagaagaaggagaagatttgcagtcctgcttgatttgcgtggcaagaagagttcccatgaaggaaagaccagttcttccctcatcagaaagttttactactcgccaggatctccaaggcaagatcacgtctctggataccagcaccatgagagcagccatgaaaccaggctgggaggacctggtaagaaggtgtattcagaagttccatgcgcagcatgaaggagaatctgtgtcctatgctaagaggcatcatcatgaagtactgagacaaggattggcattcagtcaaatctatcgtttttccttgtctgatggcactcttgttgctgcacaaacgaagagcaaactcatccgttctcagactactaatgaacctcaacttgtaatatctttacatatgcttcacagagagcagaatgtgtgtgtgatgaatccggatctgactggacaaacgatggggaagccactgaatccaattagctctaacagccctgcccatcaggccctgtgcagtgggaacccaggtcaggacatgaccctcagtagcaatataaattttcccataaatggcccaaaggaacaaatgggcatgcccatgggcaggtttggtggttctgggggaatgaaccatgtgtcaggcatgcaagcaaccactcctcagggtagtaactatgcactcaaaatgaacagcccctcacaaagcagccctggcatgaatccaggacagcccacctccatgctttcaccaaggcatcgcatgagccctggagtggctggcagccctcgaatcccacccagtcagttttcccctgcaggaagcttgcattcccctgtgggagtttgcagcagcacaggaaatagccatagttataccaacagctccctcaatgcacttcaggccctcagcgaggggcacggggtctcattagggtcatcgttggcttcaccagacctaaaaatgggcaatttgcaaaactccccagttaatatgaatcctcccccactcagcaagatgggaagcttggactcaaaagactgttttggactatatggggagccctctgaaggtacaactggacaagcagagagcagctgccatcctggagagcaaaaggaaacaaatgaccccaacctgcccccggccgtgagcagtgagagagctgacgggcagagcagactgcatgacagcaaagggcagaccaaactcctgcagctgctgaccaccaaatctgatcagatggagccctcgcccttagccagctctttgtcggatacaaacaaagactccacaggtagcttgcctggttctgggtctacacatggaacctcgctcaaggagaagcataaaattttgcacagactcttgcaggacagcagttcccctgtggacttggccaagttaacagcagaagccacaggcaaagacctgagccaggagtccagcagcacagctcctggatcagaagtgactattaaacaagagccggtgagccccaagaagaaagagaatgcactacttcgctatttgctagataaagatgatactaaagatattggtttaccagaaataacccccaaacttgagagactggacagtaagacagatcctgccagtaacacaaaattaatagcaatgaaaactgagaaggaggagatgagctttgagcctggtgaccagcctggcagtgagctggacaacttggaggagattttggatgatttgcagaatagtcaattaccacagcttttcccagacacgaggccaggcgcccctgctggatcagttgacaagcaagccatcatcaatgacctcatgcaactcacagctgaaaacagccctgtcacacctgttggagcccagaaaacagcactgcgaatttcacagagcacttttaataacccacgaccagggcaactgggcaggttattgccaaaccagaatttaccacttgacatcacattgcaaagcccaactggtgctggacctttcccaccaatcagaaacagtagtccctactcagtgatacctcagccaggaatgatgggtaatcaagggatgataggaaaccaaggaaatttagggaacagtagcacaggaatgattggtaacagtgcttctcggcctactatgccatctggagaatgggcaccgcagagttcggctgtgagagtcacctgtgctgctaccaccagtgccatgaaccggccagtccaaggaggtatgattcggaacccagcagccagcatccccatgaggcccagcagccagcctggccaaagacagacgcttcagtctcaggtcatgaatatagggccatctgaattagagatgaacatggggggacctcagtatagccaacaacaagctcctccaaatcagactgccccatggcctgaaagcatcctgcctatagaccaggcgtcttttgccagccaaaacaggcagccatttggcagttctccagatgacttgctatgtccacatcctgcagctgagtctccgagtgatgagggagctctcctggaccagctgtatctggccttgcggaattttgatggcctggaggagattgatagagccttaggaatacccgaactggtcagccagagccaagcagtagatccagaacagttctcaagtcaggattccaacatcatgctggagcagaaggcgcccgttttcccacagcagtatgcatctcaggcacaaatggcccagggtagctattctcccatgcaagatccaaactttcacaccatgggacagcggcctagttatgccacactccgtatgcagcccagaccgggcctcaggcccacgggcctagtgcagaaccagccaaatcaactaagacttcaacttcagcatcgcctccaagcacagcagaatcgccagccacttatgaatcaaatcagcaatgtttccaatgtgaacttgactctgaggcctggagtaccaacacaggcacctattaatgcacagatgctggcccagagacagagggaaatcctgaaccagcatcttcgacagagacaaatgcatcagcaacagcaagttcagcaacgaactttgatgatgagaggacaagggttgaatatgacaccaagcatggtggctcctagtggtatgccagcaactatgagcaaccctcggattccccaggcaaatgcacagcagtttccatttcctccaaactacggaataagtcagcaacctgatccaggctttactggggctacgactccccagagcccacttatgtcaccccgaatggcacatacacagagtcccatgatgcaacagtctcaggccaacccagcctatcaggccccctccgacataaatggatgggcgcaggggaacatgggcggaaacagcatgttttcccagcagtccccaccacactttgggcagcaagcaaacaccagcatgtacagtaacaacatgaacatcaatgtgtccatggcgaccaacacaggtggcatgagcagcatgaaccagatgacaggacagatcagcatgacctcagtgacctccgtgcctacgtcagggctgtcctccatgggtcccgagcaggttaatgatcctgctctgaggggaggcaacctgttcccaaaccagctgcctggaatggatatgattaagcaggagggagacacaacacggaaatattgctgacactgctgaagccagttgcttcttcagctgaccgggctcacttgctcaaaacacttccagtctggagagctgtgtctatttgtttcaacccaactgacctgccagccggttctgctagagcagacaggcctggccctggttcccagggtggcgtccactcggctgtggcaggaggagctgcctcttctcttgacagtctgaagctcgcatccagacagtcgctcagtctgttccctgcattcaccttagtgcaacttagatctctcctccccaagtaaatgttgacaggccaatttcatacccatgtcagattgaatgtatttaaatgtatgtatttaaggagaaccatgctcttgttctgttcctgttcggttccagacactggtttcttgctttgttttccctggctaacagtctagtgccaaagattaagattttatctgggggaaagaaaagaattttttaaaaaattaaactaaagatgttttaagctaaagcctgaatttgggatggaagcaggacagacaccgtggacagcgctgtatttacagacacacccagtgcgtgaagaccaacaaagtcacagtcgtatctctagaaagctctaaagaccatgttggaaagagtctccagttactgaacagatgaaaaggagcctgtgagagggctgttaacattagcaaatattttttccttgttttttctttgttaaaaccaaactggttcacctgaatcatgaattgagaagaaataattttcatttctaaattaagtcccttttagtttgatcagacagcttgaatcagcatctcttcttccctgtcagcctgactcttcccttcccctctctcattccccatactccctattttcattccttttttaaaaaataatataagctacagaaaccaggtaagccctttatttccttaaatgttttgccagccacttaccaattgctaagtattgaatttcagaaaaaaaaaatgcatttactggcaaggagaagagcaaagttaaggcttgataccaatcgagctaaggatacctgctttggaagcatgtttattctgttccccagcaactctggcctccaaaatgggagaaacgccagtgtgtttaaattgatagcagatatcacgacagatttaacctctgccatgtgttttttattttgttttttagcagtgctgactaagccgaagttttgtaaggtacataaaatccaatttatatgtaaacaagcaataatttaagttgagaacttatgtgttttaattgtataatttttgtgaggtatacatattgtggaattgactcaaaaatgaggtacttcagtattaaattagatatcttcatagcaatgtctcctaaaggtgttttgtaaaggatatcaatgccttgattagacctaatttgtagacttaagactttttattttctaaaccttgtgattctgcttataagtcatttatctaatctatatgatatgcagccgctgtaggaaccaattcttgatttttatatgtttatattctttcttaatgaaccttagaaagactacatgttactaagcaggccacttttatggttgttttt

SEQ ID NO: 128 - DNA, 1647 bp, Homo sapiens; identical to SEQ ID NO: 68

SEQ ID NO: 129 - DNA, 3606 bp, Homo sapiens
gcggaagagcagaacgtttgggagtgtgcagctcctgggcccggctcaggcccgtcgcggaggcgcggcgcaggggagcgctcgggtgggagtctcgctcctccaccagtttattgcgacgtagcatccaggcctaggcctccccgtccatccccgccggactcgggcctctggcagcagcgggtgacgcagacggaacatcatgtcgtccgcggccgagcctccgccacccccgcctcccgagagcgcgccttccaagcccgcagcctcgatcgccagcggcgggagcaacagcagcaacaaaggcggccccgaaggcgtcgcggcgcaggcggttgcgtctgcggccagcgctggtcccgcagacgccgagatggaggaaatatttgatgatgcgtcacctggaaagcaaaaggaaatccaagaaccagatcctacctatgaagaaaaaatgcaaactgaccgggcaaatagattcgagtatttattaaagcagacagaactttttgcacatttcattcaacctgctgctcagaagactccaacttcacctttgaagatgaaaccagggcgcccacgaataaaaaaagatgagaagcagaacttactatccgttggcgattaccgacaccgtagaacagagcaagaggaggatgaagagctattaacagaaagctccaaagcaaccaatgtttgcactcgatttgaagactctccatcgtatgtaaaatggggtaaactgagagattatcaggtccgaggattaaactggctcatttctttgtatgagaatggcatcaatggtatccttgcagatgaaatgggcctaggaaagactcttcaaacaatttctcttcttgggtacatgaaacattatagaaacattcctgggcctcatatggttttggttcctaagtctacattacacaactggatgagtgaattcaagagatgggtaccaacacttagatctgtttgtttgataggagataaagaacaaagagctgcttttgtcagagacgttttattaccgggagaatgggatgtatgtgtaacatcttatgaaatgcttattaaagagaagtctgtgttcaaaaaatttaattggagatacttagtaatagatgaagctcacaggatcaaaaatgaaaaatctaagttgtcagaaatagtgagggaattcaagactacaaatagactattattaactggaacacctcttcagaacaacttgcatgagctgtggtcacttcttaactttctgttgccagatgtgtttaattcagcagatgactttgattcctggtttgatacaaacaactgccttggggatcaaaaactagttgagaggcttcatatggttttgcgtccattcctccttcgtcgaattaaggctgatgttgaaaagagtttgcctccaaagaaggaagtaaaaatctatgtgggcctcagcaaaatgcaaagggaatggtatactcggatattaatgaaggatatagatatactcaactcagcaggcaagatggacaaaatgaggttattgaacatcctaatgcagttgagaaaatgttgtaatcatccatatctctttgatggagcagaacctggtccaccttatacaacagatatgcatctagtaaccaacagtggcaaaatggtggttttagacaagctgctccctaagttaaaagaacaaggttcacgagtactaatcttcagtcaaatgacaagggtattggacattttggaagattattgcatgtggagaaattatgagtactgcaggttggatggtcagacaccccatgatgagagacaagactccatcaatgcatacaatgaaccaaacagcacaaagtttgttttcatgttaagcacgcgtgctggtggtcttggcatcaatcttgcgactgctgatgtagtaattttgtatgattctgattggaatccccaagtagatcttcaggctatggaccgagcacatagaattgggcagactaagacagtcagagtgttccgctttataactgataacactgtagaagaaagaatagtagaacgtgctgagatgaaactcagactggattcaatagtcattcaacaagggaggcttgtggatcagaatctgaacaaaattgggaaagatgaaatgcttcaaatgattagacatggagcaacacatgtgtttgcttcaaaggaaagtgagatcactgatgaagatatcgatggtattttggaaagaggtgcaaagaagactgcagagatgaatgaaaagctctccaagatgggcgaaagttcacttagaaactttacaatggatacagagtcaagtgtttataacttcgaaggagaagactatagagaaaaacaaaagattgcattcacagagtggattgaaccacctaaacgagaaagaaaagccaactatgccgttgatgcatatttcagggaagctcttcgtgttagtgaacctaaagcacccaaggctcctcgacctccaaaacaacccaatgttcaggatttccagttctttcctccacgtttatttgaattactggaaaaagaaattctgttttacagaaaaactattgggtacaaggtacctcgaaatcctgagctgcctaacgcagcacaggcacaaaaagaagaacagcttaaaattgatgaagctgaatcccttaatgatgaagagttagaggaaaaagagaagcttctaacacagggatttaccaattggaataagagagattttaaccagtttatcaaagctaatgagaagtggggtcgtgatgatattgaaaatatagcaagagaagtagaaggcaaaactccagaagaagtcattgaatattcagctgtgttttgggaaaggtgcaacgagctccaggacatagagaagattatggctcagattgaaaggggagaggcgagaattcaaagaagaataagcatcaagaaagcacttgacacaaagattggacggtacaaagcaccttttcatcagctgagaatatcatatggtactaacaaaggaaaaaactatactgaagaagaagatcgttttctgatttgtatgcttcacaaacttggatttgacaaagaaaatgtttatgatgaattgcgacagtgtattcgcaactctcctcagttcagatttgactggtttcttaagtccagaactgcaatggagctccagaggagatgtaataccttaattactttgattgaaagagaaaacatggaactagaagaaaaggagaaggcagagaaaaagaaacgaggaccaaagccttcaacacagaaacgtaaaatggatggcgcacctgatggtcgaggaagaaaaaagaagctgaaactatgaatatgtttttgtttcataatcactaactttaaaccagtagttctttaatttacgggtcttcataagatgtactgtacaatgctcaattgttatgtcatttaaagacatcaggttcatctgtttactgagctagaaacatagtatgtagtttcacttttttaaatgcaacagctgtgctgaaatttttttatcattaacacttgaagtaataaaataggcttcatttattaaaaaaaaaaaaaaaa

SEQ ID NO: 130 - DNA, 5257 bp, Homo sapiens
aaaaattttctgttaccaaattttacaacttctaataagactactataactttatgtaaactgatgaagatgtgctgattaacatattctgtgatatggtttacaacttttaatcataattgtccatgattttggaatgctgttatttatcagtaaatgtaaaatatttgaggcatttagccatacacacactagaactttttaaaactttgtcctatagtgtaattataaactgatgactattatcttcatacattgagtcttcatgcatcaatgaaatgaaaaatataggagtagatgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagagaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagagttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctcatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaagaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtgaccaaatgggcctcaaagattcagattgaaacaaacaaaaagctttt

SEQ ID NO: 131 - DNA, 5247 bp, Homo sapiens
ttccggcgggggaggcgccgggaagtcgatggcgccggcggctcctgcaggaggccactgtctgcagctcccgtgaagatgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgccgcagcacaaggctgcccaggtcgccaaggagaaaaagaagaaaaagaaaaagaagaaggcagaaaatgcagaaggacagacgcctgccattgggccggatggcgagcctctagacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgagagcagacactgcagcacgggcagcggcagtgccagcttcgcccacactgcccctccgccagcgggcgtcaaccccgacttggaggagccacctctaaaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagaga

SEQ ID NO: 132 - DNA, 3418 bp, Homo sapiens
attcccggggtctgactggactcgcggcgacttacctttcagtcgtgcgctcctgatccggcgctcggaatttgtccccggcttcagggctgcggggcctggaaggaggcgtatcgaggcggctcgaaaacgatccaggggagccgaggcgctcctcttgtcatcccactcagcgccatgtcctggatgttcaagagggatccagtttggaagtacttgcagactgtccagtatggagttcatggaaattttccacgcctctcatatccaactttctttccacgttttgaattccaagatgttatccctccagatgactttctaactagtgatgaagaagtagattccgttttatttggaagtttgagaggtcatgtggttggactacgctattacacgggagtagttaataataatgaaatggttgcattacaacgagatcctaataacccttatgataagaatgcaattaaagtaaacaatgtgaatggaaatcaagttggccatttaaagaaagagcttgcaggtgctttggcctatatcatggacaacaaattggcacaaattgaaggggtagttccttttggtgcaaacaatgcttttaccatgcctctgcatatgactttttggggaaaagaagaaaatagaaaagcggtttcagatcagttgaagaaacatggatttaaattgggtcctgcaccaaaaactttaggattcaatttggaaagtggttggggctctggaagagctggaccaagctatagtatgccagtgcatgctgcagtacagatgacaactgaacagcttaaaacagaatttgacaaattgtttgaagatttaaaagaagatgataaaacccatgaaatggaaccagctgaggctattgaaacaccactgcttccacatcaaaaacaagctctagcttggatggtgtcacgggaaaatagcaaagaacttccaccattctgggaacagcgaaatgacttatactataacacaataacaaatttttctgagaaggaccgaccagaaaatgtccatggaggaattttagctgatgatatgggtttgggtaaaactcttacagccattgcagtaatccttaccaacttccatgatggcagacctcttcctattgaaagagttaaaaagaatctactgaagaaggaatataatgttaacgatgactctatgaaacttggaggaaacaataccagtgaaaaggcagatggactaagcaaagacgcatctagatgtagtgaacaacccagtatttcagatatcaaggagaagagtaagtttcgcatgtcagaattgtctacgtcccgccccaaaagaagaaaaactgctgtccagtacatagaaagcagtgattcagaggaaattgaaacaagtgaattgccgcagaaaatgaaaggcaaactgaaaaatgtacagtctgaaactaaaggcagggcgaaagcaggatcttctaaggttatagaagatgtggcatttgcatgtgcattaacttcatctgttcctacaacaaaaaagaaaatgttgaaaaagggagcttgtgcagtggaggggtcaaagaaaactgatgttgaggagagaccaagaacaacactgatcatctgtccgctttctgtgttaagcaactggattgaccagtttggacaacatataaaatcagatgtacacttgaatttttatgtttattatggtcctgatcgtattagagaaccggccttactttcaaaacaggatattgttttgactacgtataatattttaactcatgactatggaactaaaggagatagtccattacatagcataaggtggctaagagtgatcctggatgaaggacatgccatacgaaatccaaatgctcagcagacaaaagctgtacttgacttagaatcagaaagaagatgggttttgacaggtactccaatccagaattctttaaaggacttgtggtctcttctttcctttttaaaacttaaaccatttattgatagagaatggtggcatagaacaatacagcgtcctgtcacaatgggagatgaaggaggacttaggcgtttacagtccctaattaaaaatattacacttagaagaacaaagacaagcaaaattaaaggaaaacctgttttggagttaccagaacgtaaagtatttattcagcacattacactttcagatgaagagagaaagatttatcagtctgtgaaaaatgaaggcagagccactattggaaggtattttaatgaagggactgtcctggcacattatgcagatgtcctgggtcttttgcttagactgcggcaaatttgttgccatacttaccttcttacaaatgcagtgtcttccaatggcccctcaggaaatgatacacctgaagaactgagaaagaagttaataaggaagatgaagttaattctgagctcaggttcagatgaggaatgtgcaatttgcctggattctttaacagttcctgtgataacacattgtgcacatgtattttgtaaaccctgtatttgccaagtcattcagaatgagcagccacatgctaaatgccctttatgcagaaatgatatacatgaagataatttattagaatgtcctccagaagaattagcacgtgacagtgagaaaaagtctgatatggaatggacatccagttcaaagattaatgcgctaatgcacgcattgactgacttaagaaagaagaatcccaacataaaaagtttggttgtttctcagtttacaacattcctgtctttaatagaaataccacttaaagcctctggatttgtgtttactcgtttggatggttccatggcccaaaagaaaagagttgaatcaattcagtgttttcaaaacactgaagcaggatctccaactataatgcttctgtccttaaaagcaggtggagttggtttgaatctgtctgcagcttctcgagtgtttttaatggatccagcctggaatcctgctgctgaagatcagtgctttgacagatgccatagacttggtcagaagcaagaagttatcatcacaaaattcattgtaaaggactctgttgaagaaaatatgctgaaaatacaaaacaaaaagagagaacttgcagcaggagcctttggaactaaaaaaccaaatgctgacgaaatgaaacaagccaaaattaatgaaatcagaacattaattgacttataatttgtgggattttagtaagaagactactatatgtgagaggcgtgatatctggatggaagttgggctggatgatctccaaagtcgtttcaactcttaaagacatcttaatcctgaatgtaaacaattgttatgtgtttagaatcagaatttgattttgaacttgagtaattcatccttacagctatctgtagaattagtcatcttttttctt

SEQ ID NO: 133 - DNA, 3022 bp, Homo sapiens
gctgggttggaaaaagactatgttagcaagtgtcacgccatgcttttgccaactttccaattaaaggttgacattcctgcataaacatttctctgtgaaaatgtccttgcctcttacagaggagcagaggaaaaagattgaagagaatcgacaaaaggctctggcccgcagagctgagaagttattggcagaacagcatcagaggactagctcgggcacctccattgctggcaacccattccaggccaagcaaggcccatcccaaaatttcccaagggagtcttgtaagccagtgagccatggtgtcattttcaagcaacagaatctcagtagctcatctaatgctgaccaaagacctcatgattcccacagttttcaggcaaagggaatatggaaaaagccagaagaaatgcccacagcctgcccaggccacagtccacgtagtcaaatgactctcactggaatctctcctcccttggcacaaagtcctccagaggtccctaaacaacagctcttgagttatgagttaggtcaaggtcatgctcaggcttcacctgagatcaggttcacaccctttgctaacccaactcataagcctctggccaaaccaaagagttcccaagagacaccagctcattcctctggacagcctcccagggatgctaagttagaggccaagacagcaaaagcctccccttcggggcagaacatttcttacatccattctagctcagagagtgtaacgcccaggacagaaggaagactccagcagaagtcagggtcctcagtccaaaaaggagtgaactctcagaagggaaagtgcgtaaggaacggcgatcgtttccaggtgttgattgggtacaatgcggaactcattgcagtgtttaagaccctgcccagcaagaattatgatcctgacaccaagacgtggaacttcagcatgaatgactatagtgccctgatgaaagcagcccagagcctccccacggtcaacctgcagcctctggaatgggcctatgggagcagcgagtcaccctccaccagcagtgagggacaggccggccttccatcagctccatccctttcatttgtcaaagggcgaggcatgctcatctccagggcctacttcgaggcagacatcagttattcacaggaccttattgcgctttttaaacagatggattccagaagatatgatgtcaagaccaggaagtggagctttctcttggaagagcacagtaaactaattgcaaaggtgcgctgcctcccacaagttcagctggaccctctgcccacgactctcaccctggcgtttgcttctcagctcaagaagacatctctcagtctcacgccagatgtcccagaggcagacctttctgaagtggaccccaagctcgtgtctaatctgatgccctttcagagagctggagtcaattttgccatagccaaaggaggccgcctgctgctcgctgacgacatgggcctggggaagaccatccaagccatctgcatcgcagccttttaccggaaggagtggccgctcctggtggtggtgccatcctccgtgcgcttcacctgggagcaggccttccttcggtggctgccatctctgagcccagattgcatcaacgtcgtggtgactgggaaggaccgcctgacagctggcctgatcaacattgtcagctttgaccttcttagcaagttggaaaaacagctaaaaaccccttttaaagttgtcatcattgatgaatctcacttcctcaaaaacagtaggactgcccgctgtcgagcagctatgccggtcctaaaggtcgccaagagggtgatcctgttgtcgggcacaccagccatgtcccggcccgcagagctctacacgcagatcatcgcagtcaagccaactttcttcccccagtttcatgcctttggacttcgctactgtgatgccaaacggatgccttgggggtgggactactcaggttcctccaacctgggagagctgaagctcctgctggaggaagcagtcatgctgcggcgcctcaagtccgacgtcctttcccagctgcctgccaagcagcgcaagatagtggtgattgccccaggacggatcaatgccaggaccagagctgccctggatgctgcagccaaggaaatgaccaccaaggacaaaactaaacagcagcagaaagatgccctcattctcttcttcaacagaacagctgaagctaaaatcccatctgtcattgaatatatcttggacctactggaaagtggaagagagaagtttttagtatttgcacaccataaggtggtcctggacgcaattacgcaagagcttgagagaaagcacgtgcagcacatccgcatcgatggctccacctcatcagctgagcgggaggacctgtgccagcagttccaactgtcggagaggcatgctgtggccgtgctgtccatcaccgctgccaatatgggcctcaccttctcctcggctgacctggtggtgtttgctgagctgttttggaacccaggggtgctgatccaggctgaggaccgcgtgcaccgcattggacagaccagctccgtgggcattcactacctcgtggcaaagggcacagctgatgactacctttggcccctgattcaagagaagattaaagttctggcagaagccgggctttctgagaccaatttttcagaaatgacagaatccactgattacctctacaaggacccaaagcagcagaagatgtacgacctattccagaagtcctttgagaaagaaggaagtgatatggagctcgtggaagcagcagagtcctttgacccaggaagtggttcaggaacatctggaagtagttcccagaacatgggagacaccctggatgaaagctcattgacagccagtccacagaagaaaaggagatttgaattttttgataactgggacagctttacgtctcccctgtaaaaggggcaaaaaaaaaaaaaaaaaaagcattttaaaatcaaaaagaaaaaaataaaa

SEQ ID NO: 134 - DNA, 3634 bp, Homo sapiens
caagactggaagcagagagagagagcaagagtgagagagagcgagcgagcgtagtcaggagatggtgttgtattccaggagaaaaacgtttcttcatttcaactcaaacttgctgctaaagcgcctaaatctgaaaaggaaatggacccagaatatgaagagaaaatgaaagccgaccgagcaaagagatttgaatttttactgaagcagacagaactttttgcacatttcattcagccttcagcacagaaatctccaacatctccactgaacatgaaattgggacgtccccgaataaagaaagatgaaaagcagagcttaatttctgctggagactaccgccataggcgcacagagcaagaagaagatgaagagctactgtctgagagtcggaaaacatctaatgtgtgtattagatttgaggtgtcaccttcatatgtgaaaggggggccactgagagattatcagattcgaggactgaattggttgatctctttatatgaaaatggagtcaatggcattttggctgatgaaatgggccttgggaaaactttacaaacaattgctttgcttggttacctgaaacactaccgaaatattcctggacctcacatggttttagttccaaagtctactttacacaactggatgaatgaatttaaacgatgggtcccatctctccgtgtcatttgttttgtcggagacaaggatgccagagctgcttttattcgtgatgaaatgatgccaggagagtgggatgtttgcgttacttcttatgagatggtaattaaagaaaaatctgtattcaaaaagtttcactggcgatacctggtcattgatgaagctcacagaataaagaatgaaaaatctaagctttcagagattgttcgtgagttcaagtcgactaaccgcttgctcctaactggaacacctttgcagaataacctgcatgaactgtgggccttactcaactttttattgcctgatgtctttaattctgcagatgactttgattcttggtttgacactaaaaattgtcttggtgatcaaaaactcgtggaaagacttcatgcagttttaaaaccatttttgttacgccgtataaaaactgatgtagagaagagtctgccacctaaaaaggaaataaagatttacttggggctgagtaagatgcaacgagaatggtatacaaaaatcctgatgaaagatattgatgttttaaactcttctggcaagatggacaagatgcgactcttaaacattctgatgcagcttcgaaagtgttgtaatcatccatatctgtttgatggtgctgaacctggtccaccttataccactgatgagcatattgtcagcaacagtggtaaaatggtagttctggataaactattggccaaactcaaagaacagggttcaagggttctcattttcagccagatgactcgcttgctggatattttggaagattattgcatgtggcgtggttatgagtattgtcgactggatggacaaaccccgcatgaagaaagagaggataaattcctagaagtggaatttctgggtcaaagggaagcaatagaggcttttaatgctcctaatagtagcaaattcatctttatgctaagtaccagggctggaggtctcggaattaacctggcaagtgctgatgtggttatactatatgattcagactggaacccacaggttgatctacaagctatggatcgagcacatcgtattggtcagaagaaaccagtacgtgtattccgtctcatcactgacaacactgttgaagagaggattgtagaaagagctgagataaaactgagactcgattcaattgttatacaacaaggaagactcattgaccaacggtctaacaagctggcaaaagaggaaatgttacaaatgatacggcatggagccacccatgtttttgcttctaaagagagtgagttgacagatgaagacattacaactattctggaaagaggggaaaagaagactgcagagatgaatgaacgcctgcaaaaaatgggagagtcttctctaagaaattttagaatggacattgaacaaagtttatacaaatttgagggagaagattatagagaaaaacagaagcttggcatggtggaatggattgaacctcctaaacgagaacgcaaagcaaactacgcagtggatgcctactttagagaggctttgcgtgtcagcgagccaaagattccaaaggctccacggcctccaaaacagccaaatgttcaggattttcaatttttcccaccacgcttatttgagctcctggaaaaggaaattctttattatcggaagacaataggctataaggttccaaggaatcctgatatcccaaatccagctctggctcaaagagaagagcaaaaaaagattgatggagctgaacctcttacaccagaagagactgaagaaaaggaaaaacttctcacacaaggtttcacaaactggactaaacgagattttaaccagtttattaaagctaatgagaaatatggaagagatgacattgataacatagctcgagaggtagagggcaaatcccctgaggaggtcatggagtattcagctgtattttgggaacgttgcaatgaattacaggacattgagaaaattatggctcaaattgaacgtggagaagcaagaattcaacgaaggatcagtatcaagaaagccctggatgccaaaattgcaagatacaaggctccatttcatcagttgcgcattcagtatggaaccagcaaaggaaagaactatactgaggaagaagatagattcttgatttgtatgttacacaaaatgggctttgatagagaaaatgtatatgaagaattaagacagtgtgtacgaaatgctccccagtttagatttgactggtttatcaagtctaggactgccatggaattccagagacgctgtaacactctgatttcattgattgagaaagaaaatatggaaattgaggaaagagagagagcagaaaagaagaaacgggcaactaaaactccaatggtaaaattttcagcattttcctaacttttagatttaacattgttgggccatttaaaatgtgcatattggagcagaacattaaatctgtttccattttagtcacagaaaagaaaagcagagtcagctactgagagctctggaaagaaggatgtcaagaaggtgaaatcctaaagcctagaaataaagttttaaatgggaaactgctattttcttgttcccatcttcaaatgctaattgccagttccagtgtattcatggtactctaagaaaaatctctttggttttgatttcttgcatattttatatattttacaatgctttctacctgaaatgtgtagctttatattttatggcattctagtatttttgtgtactgtattttgtgcatttcatgtcttcatcaaaatcctctcagtccttgttcttttgaagcttgtgctgaggttttagcttttctatgttttatatgccgctgctttgaaagagaacctagattctatagttgtattattgttgtttcatactttaaatttatatggctgtggaaaaacgaattaaaatgttttgaggagaaagaaaaaaaaaa

SEQ ID NO: 135 - DNA, 396 bp, Homo sapiens
atggcggacgtggtcgtgggtaaagacaagggcggggagcagcggctcatctcgctgcctctatcccgcatccgggtcatcatgaagagctcccccgaggtgtccagcatcaaccaggaggcgttggtgctcacggccaaggccacggagctctttgttcaatgcctagccacctattcctacagacacggcagtggaaaggaaaagaaagtactgacttacagtgatttagcaaacactgcacagcaatcagaaacttttcagtttcttgcagatatattaccaaagaagattttagctagtaaatacctgaaaatgcttaaagaggaaaagagggaagaagatgaggagaatgacaatgataatgaaagtgaccatgatgaagctgactcctaa

(19) United States
(12) Patent Application Publication
(10) Pub. No.: US 2007/0087991 A1
(43) Pub. Date: Apr. 19, 2007

(54) Pluripotential stem cells

(75) Inventors: Peter Andrews, Sheffield (GB); James Walsh, Sheffield (GB); Paul Gokhale, Sheffield (GB)

Correspondence Address: CROWELL & MORING LLP;INTELLECTUAL PROPERTY GROUP, P.O. BOX 14300, WASHINGTON, DC, 20044-4300, US

(73) Assignee: AXORDIA LIMITED, Sheffield (GB)

(21) Appl. No.: 11/600,125

(22) Filed: Nov. 16, 2006

Related U.S. Application Data
(63) Continuation of application No. 10/504,173, filed on Apr. 22, 2005, now abandoned.

(30) Foreign Application Priority Data
Feb. 13, 2002 (GB) 0203359.5
Feb. 13, 2002 (GB) 0203387.6

(51) Int. Cl. A61K 48/00; C07H 21/02; C12N 5/08

(52) U.S. Cl. 514/44; 435/455; 435/366; 536/23.1

(57) ABSTRACT

We describe a method to manipulate the phenotype of stem cells, preferably pluripotential stem cells including nucleic acids and vectors used in said methods.

The invention relates to a method to manipulate the phenotype of stem cells, preferably pluripotential stem cells and including nucleic acids and vectors used in said methods.

A number of techniques have been developed in recent years which purport to specifically ablate genes and/or gene products. For example, the use of anti-sense nucleic acid molecules to bind to and thereby block or inactivate target mRNA molecules is an effective means to inhibit the production of gene products. This is typically very effective in plants where anti-sense technology produces a number of striking phenotypic characteristics. However, antisense is variable leading to the need to screen many, sometimes hundreds of, transgenic organisms carrying one or more copies of an antisense transgene to ensure that the phenotype is indeed truly linked to the antisense transgene expression. Antisense techniques, not necessarily involving the production of stable transfectants, have been applied to cells in culture, with variable results.

In addition, the ability to be able to disrupt genes via homologous recombination has provided biologists with a crucial tool in defining developmental pathways in higher organisms. The use of mouse gene “knock out” strains has allowed the dissection of gene function and the probable function of human homologues to the deleted mouse genes, (Jordan and Zant, 1998).

A much more recent technique to specifically ablate gene function is through the introduction of double stranded RNA, also referred to as inhibitory RNA (RNAi), into a cell which results in the destruction of mRNA complementary to the sequence included in the RNAi molecule. The RNAi molecule comprises two complementary strands of RNA (a sense stand and an antisense strand) annealed to each other to form a double stranded RNA molecule. The RNAi molecule is typically derived from exonic or coding sequence of the gene which is to be ablated.

Recent studies suggest that RNAi molecules ranging from 100-1000 bp derived from coding sequence are effective inhibitors of gene expression. Surprisingly, only a few molecules of RNAi are required to block gene expression which implies the mechanism is catalytic. The site of action appears to be nuclear as little if any RNAi is detectable in the cytoplasm of cells indicating that RNAi exerts its effect during mRNA synthesis or processing.

The exact mechanism of RNAi action is unknown although there are theories to explain this phenomenon. For example, all organisms have evolved protective mechanisms to limit the effects of exogenous gene expression. For example, a virus often causes deleterious effects on the organism it infects. Viral gene expression and/or replication therefore needs to be repressed. In addition, the rapid development of genetic transformation and the provision of transgenic plants and animals has led to the realisation that transgenes are also recognised as foreign nucleic acid and subjected to phenomena variously called quelling (Singer and Selker, 1995), gene silencing (Matzke and Matzke, 1998), and co-suppression (Stam et. al., 2000).

Initial studies using RNAi used the nematode Caenorhabditis elegans. RNAi injected into the worm resulted in the disappearance of polypeptides corresponding to the gene sequences comprising the RNAi molecule (Montgomery et. al., 1998; Fire et al., 1998). More recently the phenomenon of RNAi inhibition has been shown in a number of eukaryotes including, by example and not by way of limitation, plants, trypanosomes (Shi et. al., 2000) Drosophila spp. (Kennerdell and Carthew, 2000). Recent experiments have shown that RNAi may also function in higher eukaryotes. For example, it has been shown that RNAi can ablate c-mos in a mouse ooctye and also E-cadherin in a mouse preimplanation embryo (Wianny and Zernicka-Goetz, 2000).

During mammalian development those cells that form part of the embryo up until the formation of the blastocyst are said to be totipotent (e.g. each cell has the developmental potential to form a complete embryo and all the cells required to support the growth and development of said embryo). During the formation of the blastocyst, the cells that comprise the inner cell mass are said to be pluripotential (e.g. each cell has the developmental potential to form a variety of tissues).

Embryonic stem cells (S cells, those with pluripotentiality) may be principally derived from two embryonic sources. Cells isolated from the inner cell mass are termed embryonic stem (ES) cells. In the laboratory mouse, similar cells can be derived from the culture of primordial germ cells isolated from the mesenteries or genital ridges of days 8.5-12.5 post coitum embryos. These would ultimately differentiate into germ cells and are referred to as embryonic germ cells (EG cells). Each of these types of pluripotential cell has a similar developmental potential with respect to differentiation into alternate cell types, but possible differences in behaviour (e.g. with respect to imprinting) have led to these cells to be distinguished from one another.

Typically ES/EG cell cultures have well defined characteristics. These include, but are not limited to;
i) maintenance in culture for at least 20 passages when maintained on fibroblast feeder layers;
ii) produce clusters of cells in culture referred to as embryoid bodies;
iii) ability to differentiate into multiple cell types in monolayer culture;
iv) can form embryo chimeras when mixed with an embryo host;
v) express ES/EG cell specific markers.

Until very recently, in vitro culture of human ES/EG cells was not possible. The first indication that conditions may be determined which could allow the establishment of human ES/EG cells in culture is described in WO96/22362. The application describes cell lines and growth conditions which allow the continuous proliferation of primate ES cells which exhibit a range of characteristics or markers which are associated with stem cells having pluripotent characteristics.

More recently Thomson et al (1998) have published conditions in which human ES cells can be established in culture. The above characteristics shown by primate ES cells are also shown by the human ES cell lines. In addition the human cell lines show high levels of telomerase activity, a characteristic of cells which have the ability to divide continuously in culture in an undifferentiated state. Another group (Reubinoff et al., 2000) have also reported the derivation of human ES cells from human blastocyts. A third group (Shamblott et. al., 1998) have described EG cell derivation.

A feature of ES/EG cells is that, in the presence of fibroblast feeder layers, they retain the ability to divide in an undifferentiated state for several generations. If the feeder layers are removed then the cells differentiate. The differentiation is often to neurones or muscle cells but the exact mechanism by which this occurs and its control remain unsolved.

In addition to ES/EG cells a number of adult tissues contain cells with stem cell characteristics. Typically these cells, although retaining the ability to differentiate into different cell types, do not have the pluripotential characteristics of ES/EG cells. For example haemopoietic stem cells have the potential to form all the cells of the haemopoietic system (red blood cells, macrophages, basophils, eosinophils etc). All of nerve tissue, skin and muscle retain pools of cells with stem cell potential. Therefore, in addition to the use of embryonic stem cells in developmental biology, there are also adult stem cells which may also have utility with respect to determining the factors which govern cell differentiation. Further recent studies have suggested that some stem cells previously thought to be committed to a single fate, (e.g. neurons) may indeed possess considerable pluripotentcy in certain situations. Neural stem cells have recently been shown to chimerise a mouse embryo and form a wide range of non-neural tissue (Clark et. al., 2000).

A further group of cells which have relevance to developmental biology are teratocarcinoma cells (EC cells). These cells form tumours referred to as teratomas and have many features in common with ES/EG cells. The most important of these features is the characteristic of pluripotentiality.

Teratomas contain a wide range of differentiated tissues, and have been known in humans for many hundreds of years. They typically occur as gonadal tumours of both men and women. The gonadal forms of these tumours are generally believed to originate from germ cells, and the extra gonadal forms, which typically have the same range of tissues, are thought to arise from germ cells that have migrated incorrectly during embryogenesis. Teratomas are therefore generally classed as germ cell tumours which encompasses a number of different types of cancer. These include seminoma, embryonal carcinoma, yolk sac carcinoma and choriocarcinoma

The similar biology of EC cells with ES/EG cells has been exploited to study the developmental fates of cells and to identify cell markers commonly expressed in EC cells and ES/EG cells. For example, and not by way of limitation, the expression of specific cell surface markers SSEA-3 (+), SSEA-4 (+), TRA-1-60 (+), TRA-1-81 (+) (Shevinsky et al 1982; Kannagi et al 1983; Andrews et al 1984a; Thomson et al 1995); alkaline phosphatase (+) (Andrews et. al., 1996); and October 4 (Scholer et. al., 1989; Kraft et. al., 1996; Reubinoff et. al., 2000; Yeom et. al., 1996).

It is well known that gene expression can be affected at many levels. For example, at the level of transcription, translation or post-translationally by modifications to proteins which confer an altered biological activity to the modified protein. It is also known that the way in which DNA is packaged as chromatin can influence the expression of genes.

There are several levels of structural packaging of DNA leading from a double stranded helix to a mitotic chromosome, after which the DNA is some ˜50,000 times shorter than its extended length (Alberts et al., 1998). Double-stranded helical DNA is wound around the structural unit of a nucleosome, comprising an octamer core composed of 4 types of histones: two each of the H2A, H₂B, H3, and H4 proteins. Approximately 166 base pairs are bound to the nucleosome through electrostatic forces between the negatively charged phosphate groups in the DNA backbone and positively charged amino acids (e.g., lysine and arginine) in the histone proteins (Wolfe, 1993). Whilst the majority of the base pairs are tightly bound to the octamer core, the remaining linker DNA (8-100 bp) hat separates adjacent core particles is associated with the H1 histone or a related “linker” histone (Finch and Klug, 1976; Thoma et al., 1979; Wolfe, 1993).

Nucleosomes are organised into the next structural level of the chromatin fibre, also referred to as a solenoid. Chromatin structure is not static and the regulated alteration in structure is termed ‘chromatin remodelling’. This process has been defined as any event that alters the nuclease sensitivity of a region of chromatin, and can occur independently or in concert with processes such as transcription (Aalfs & Kingston, 2000). For a comprehensive review of chromatin remodelling see Aalfs & Kingston, 2000.

Reversible acetylation of evolutionary conserved lysine residues in core histone proteins plays a critical role in transcriptional regulation, cell cycle progression, and developmental events. The steady state of histone acetylation is controlled by the enzymatic activities of multiple histone acetyltransferases (HATs) and histone deacetylases (HDACs). Histone hyperacetylation is associated with transcriptional activity while histone hypoacetylation correlates with transcriptional quiescence and so histone deacetylases can be considered as enzymatic transcriptional repressors. Histone deacetylases were first described by Inove & Fujimoto, 1969.

In general, histone deacetylases do not target genes directly through specific DNA-binding sites. Rather, deacetylases are localized to genes targeted for repression as part of a protein complex. Other proteins that are part of this complex, termed co-repressors, are responsible for targeting the genes to be repressed. A large number of such co-repressors have been identified to date, including the thyroid hormone receptor, Sin3, SMRT, mYY1, and MeCP2, for a comprehensive review see Pazin & Kadonaga, 1997.

In humans, four highly homologous class I HDAC enzymes (HDAC1, HDAC2, HDAC3, and HDAC8) have been identified to date, with HDAC1, HDAC2 and HDAC3 being ubiquitously expressed in many different cell types (Yang et al., 1997 and 2002). HDAC1 and HDAC2 are the human orthologues of the yeast transcriptional regulator RPD3. Analysis of the predicted amino acid sequence of HDAC3 revealed an open reading frame of 428 amino acids with a predicted molecular mass of 49 kDa.

The HDAC3 protein is 50% identical in DNA sequence and 53% identical in protein sequence compared with the previously cloned human HDAC1. Comparison of the HDAC3 sequence with human HDAC2 also yielded similar results, with 51% identity in DNA sequence and 52% identity in protein sequence (Yang et al., 1997). The expressed HDAC3 protein is functionally active because it possesses histone deacetylase activity, represses transcription when tethered to a promoter, and binds transcription factor YY1. Although HDAC3 shares some structural and functional similarities with other class I HDACs, it exists in multi-subunit complexes separate and different from other known HDAC complexes, implying that individual HDACs might function in a distinct manner (Yang et al., 2002). Within the HDACs there are three regions of highly conserved amino acid residues; histidines, aspartates and glycines, irrespective of the highly divergent nature of the C-terminal regions (Hassig et al., 1998). It is presumed that these regions form part of the active site and are also involved in maintaining interactions between HDACs and members of the co-repressor complex.

In Drosophila, active and silent states of developmentally regulated loci are maintained by trithorax and Polycomb group of proteins. Proteins of the polycomb and trithorax groups act to remodel chromatin by altering the accessibility of DNA to factors required for gene transcription. The PcG proteins are required to maintain the transcriptionally inactive state, whereas the trxG proteins are necessary to counteract silencing and maintain the transcriptionally active state. Both PcG and trxG proteins are thought to function by establishing closed or open chromatin configurations at their target genes

In humans, homologues of the members of the Drosophila polycomb group (Pc-G) proteins include; the YY1 transcription factor (YY1), the chromobox 2 gene (CBX1) and the PHD finger protein 1, transcript variant 2 (PHF1) gene. Pc-G proteins are usually considered to be inhibitors of homeotic genes. Pc-G mutants were originally identified on the basis of their causing expression of homeotic genes in unusual (ectopic) locations. This ectopic expression of genes was attributed to the failure of proper gene silencing. Pc-G proteins themselves are unable to bind to DNA, their action is dependent on their association with other chromosomal proteins, especially histones. It is suggested that the initial repression of a gene is carried out by transcription factors which have the ability to recognize DNA and that Pc-G proteins then provide a mechanism where this initial repression becomes permanent by assembling at this site and forming a multiprotein complex involved in modifying chromatin.

In humans, homologues of the members of the Drosophila Trithorax (DIN proteins, for example an enhancer of polycomb 1 (EPC1), a zinc finger protein 144 (MBL18) and a myeloid/lymphoid or mixed lineage leukemia 1 (MLLT1) are considered to be activators of homeobox genes. Mutations within trx genes result in transformations of body structures reminiscent of loss-of-function mutations in homeotic genes. For example, in the Drosophila, after the disappearance of the transiently acting patterning factors such as those encoded by the segmentation genes, maintenance of the initial transcriptional patterns of homeotic genes requires the expression of the trx gene. (Orlando et al., 1998). Genetic analyses indicate that trx expression is required continuously throughout Drosophila development, consistent with its maintenance function, but there also appears to be a critical early requirement, which if compromised cannot be compensated by subsequent continuous expression (Ingham and Whittle, 1980). Comparatively little is known about the molecular environment in which the TRX protein is integrated. However, an elucidation of this issue could be particularly rewarding as chromosomal aberrations involving the human homologues of TRX (MLL, ALL-1, HRX) is one of the most frequent genetic changes in infant leukemias of myeloid and lymphoid lineage and in treatment-induced secondary leukemias (Orlando et al., 1998).

Whereas some multi-protein complexes which alter transcriptional regulation and chromatin remodelling are based on the covalent modification of the histones (e.g. histone acetyltransferases (HATs) and histone deacetylases, others appear to function by altering chromatin structure in an ATP dependent fashion (e.g. the yeast SWI/SNF complex). A group of enzymes referred to as ATP-dependent chromatin remodellers, use the energy of ATP hydrolysis to alter interactions between DNA and histone proteins. The protein complexes that mediate ATP-dependent nucleosome remodelling and histone acetylation/deacetylation in the regulation of transcription were initially considered to be different, although it has recently been suggested that their activities might be coupled. Examples of human ATP-dependent chromatin remodellers include, SMARCA5, a human SWI/SNF related, matrix associated and actin dependent regulator of chromatin, identified as member 5 of subfamily ‘a’ of the SMARC family.

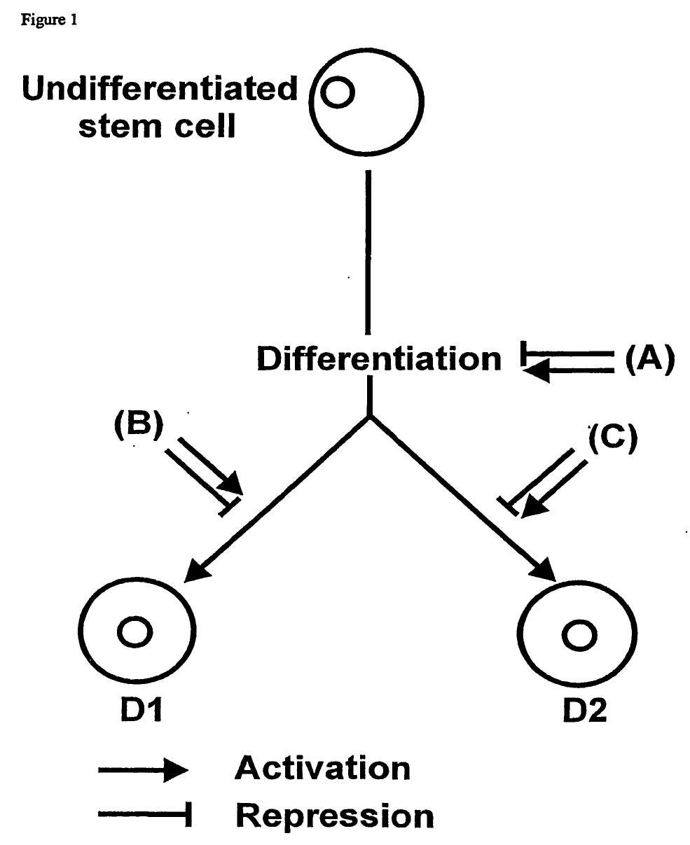

There are further examples of regulators of gene expression. We have accumulated expression studies which identify a number of genes thought to be involved in determining the developmental fate of stem cells, particularly embryonic stem cells. By northern blotting we have identified the expression of human homologs of two signalling pathways believed to be critical in cell fate determination. Expression of ligands, receptors and downstream components of the Notch and Wingless signaling cascades have been elucidated. Using the model system NTERA2/D1 embryonal carcinoma cells we have recorded changes in the expression of some of these components as the cells differentiate. Bearing in mind the role these cascades play in embryonic development throughout the animal kingdom, these changes suggest a significant role for both the wingless and Notch signalling pathways in differentiation of stem cells. Furthermore the activity of some genes are required for differentiation to occur along specific pathways e.g. the myogenic gene MyoD1. Other genes have activity which inhibits cellular differentiation along particular pathways. We envisage regulation of stem cell differentiation to yield a specific cell type could be achieved by:
(i) inhibition of certain genes that normally promote differentiation along particular pathways; therefore promoting differentiation to alternate cell phenotypes;
(ii) inhibition of gene activity that prevents differentiation into particular cell types; and
(iii) a combination of (i) and (ii), see FIG. 1

The differentiation of stem cells during embryogenesis, during tissue renewal in the adult and wound repair is under very stringent regulation: aberrations in this regulation underlie the formation of birth defects during development and are thought to underlie cancer formation in adults. Generally, it is envisaged that such stem cells are under both positive and negative regulation which allows a fine degree of control over the process of cell proliferation and cell differentiation: excess proliferation at the expense of cell differentiation can lead to the formation of an expanding mass of tissue—a cancer—whereas express differentiation at the expense of proliferation can lead to the loss of stem cells and production of too little differentiated tissue in the long term, and especially the loss of regenerative potential. Certain genes have already been identified to have a negative role in preventing stem cell differentiation. Such genes, like those of the Notch family, when mutated to acquire activity can inhibit differentiation; such mutant genes act as oncogenes. On the contrary, loss of function of such genes on their inhibition results in stem cell differentiation. We propose to use EC cells as our model cell system to follow the effects of RNAi on cell fate.

In our co-pending application, WO2/16620, discloses RNAi molecules derived from the following nucleic acid sequences which encode the following polypeptides; human Notch 1 (bNotch); hNotch 2; bNotch 3; bNotch 4; TLE-1; TLE-2; TLE-3; TLE-4; TCF7; TCF7L1; TCFFL2; TCF3; TCF19; TCF1; mFringe; lFringe; rFringe; sel 1; Numb; Numblike; LNX; FZD1; FZD2; FZD3; FZD4; FZD5; FZD6; FZD7; FZD8; FZD9; FZD10; FRZB, D11-1; D113; D114; Dlk-1; Jagged 1; Jagged 2; Wnt 1; Wnt 2; Wnt 2b; Wnt 3; Wnt 3a; Wnta; Wnt6; Wnt7a; Wnt7b; Wnt8a; Wnt8b; Wnt10b; Wnt11; Wnt14; Wnt15, SFRP1; SFRP2; SFRP4; SFRP5; SK; DKK3; CER1; WIF-1; DVL1; DVL2; DVL3; DVL1L1; mFringe; lFringe; rFringe; sel11; Numb; LNX Oct4; NeuroD1; NeuroD2; NeuroD3; Brachyury, MDFI, CIR, DLK1; Oct 4; RBPJK. The present application disclaims these genes the sequences of which are disclosed in WO02/16620.

One further family of genes are the HES and related genes which are direct targets of Notch signaling. Binding of Notch ligands to Notch receptor causes proteolytic cleavage of the receptor Mumm and Kopan, 2000). The cleaved receptor known as Notch-intracellular domain (NICD) translocates to the nucleus and binds to RBP-Jκ. This binding changes RBP-Jκ from a repressor to an activator of its target genes. The target genes are homologs of the genes found at the Drosophila Enhancer of Split complex (E(spl)). These basic helix-loop-helix (bHLH) transcription factors act as repressors of downstream tissue specific transcription factors and as such act as notch effectors. The Notch signaling through E(spl) complex genes represses certain tissue specific transcription factors.

The E(spl) family of proteins are class three bHLH factors. These include: HES1, HES2, HES4, HES6, HES7, HERP1, HERP2, HESR1, HEY1, HEY2, HEYL HRT1, HRT2, HRT3 CHF1, CHF2 GRIDLOCK.

The various members of the HES related genes encode proteins that are homologous in key motif regions. They all contain Basic helix-loop-helix and a so called orange domain. HES family members contain a terminal WRPW domain and HEY family proteins contain YRPW or closely related residues. FIG. 1 shows an alignment of human HES related proteins illustrating the major domains contained in the HES related proteins. ES/EC differentiation go through a precursor stage for example neural differentiation (Przyborski et. al., 2001) during differentiation to the numerous lineages that can form in vitro.

Notch signaling through E(spl) homologs possibly allows precursor cells to remain as precursors. In addition Notch may also play an instructive role in specifying cell types. for example (Hojo et al., 2000). Manipulation of the E(spl) homologs and other downstream targets which directly affect these processes would alter the notch signaling in target cells. This in turn would alter the balance between cells types. This could be manipulated to for example block a particular cell type forming by stopping the instructive sign g or by increasing or removing the precursor cells from the cultures. The E(spl) complex genes are potential targets which would allow cell type specific disruption of Notch signaling in differentiating cultures of stem cells.

According to an aspect of the invention there is provided a method to modulate the differentiation state of a stem cell comprising the steps of:

i) contacting a stem cell with at least one inhibitory RNA molecule (RNAi) comprising a sequence of a gene which mediates at least one step in the differentiation of said cell;

(ii) providing conditions conducive to the proliferation of the cell treated in (i) above; and optionally

(iii) maintaining and/or storing said cell.

The term modulate includes both promoting or inducing the differentiation of a stem cell into a lineage restricted stem cell or a differentiated cell or to maintain a stem cell as a stem cell with characteristics which are typical of stem cells, particularly embryonic stem cells. For example, maintenance in culture for at least 20 passages when maintained on fibroblast feeder layers; production of embryoid bodies in culture, the ability to differentiate into multiple cell types in monolayer culture; can form embryo chimeras when mixed with an embryo host; and express ES/EG cell specific markers.

In a preferred method of the invention said method is an in vitro method.

In an alternative preferred method said method is an in vivo method.

In a further preferred method of the invention said stem cell is selected from the group consisting of: haemopoietic stem cells; neural stem cells; bone stem cells; muscle stem cells; mesenchymal stem cells; trophoblastic stem cells; epithelial stem cells (derived from organs such as the skin, gastrointestinal mucosa, kidney, bladder, mammary glands, uterus, prostate and endocrine glands such as the pituitary); endodermal stem cells (derived from organs such as the liver, pancreas, lung and blood vessels); embryonic stem (ES) cells; embryonal germ (EG) cells.

In a further preferred method of the invention said stem cells are embryonal carcinoma cells. Preferably said embryonal carcinoma cells are TERA2 cells. Ideally said embryonal carcinoma cells are NTERA 2 cells.

In a further preferred method of the invention said stem cell is an embryonic stem cell or embryonal germ cell or an embryonal carcinoma cell.

In a preferred method of the invention said gene is involved in Notch/Wnt signalling.

In a preferred method of the invention said RNAi molecule is derived from a nucleic acid molecule comprising a nucleic acid sequence selected from the group consisting of:
i) a nucleic acid sequence as represented by table 1, or fragment thereof;
ii) a nucleic acid sequence which hybridises to the nucleic acid sequence of table 1 and which modulates stem cell differentiation;
iii) a nucleic acid sequence which comprise sequences which are degenerate as a result of the genetic code to the nucleic acid sequences defined in (i) and (ii).

In a preferred method of the invention said hybridisation conditions are stringent hybridisation conditions.

Typically, hybridisation conditions uses 4-6×SSPE (20×SSPE contains 175.3 g NaCl, 88.2 g NaH₂PO₄H₂O and 7.4 g EDTA dissolved to 1 litre and the pH adjusted to 7.4); 5-10× Denhardts solution (50× Denhardts solution contains 5 g Ficoll (type 400, Pharmacia), 5 g polyvinylpyrrolidone abd 5 g bovine serum albumen; 100 μg-1.0 mg/ml sonicated salmon/herring DNA; 0.1-1.0% sodium dodecyl sulphate; optionally 40-60% deionised formamide. Hybridisation temperature will vary depending on the GC content of the nucleic acid target sequence but will typically be between 42°-65°. It is well known in the art that optimal hybridisation conditions can be calculated if the sequences of the nucleic acid is known For example, hybridisation conditions can be determined by the GC content of the nucleic acid subject to hybridisation. Please see Sambrook et al (1989) Molecular Cloning; A Laboratory Approach A common formula for calculating the stringency conditions required to achieve hybridisation between nucleic acid molecules of a specified homology is:
Tₘ=81.5° C.+16.6 Log [Na⁺]+0.41[% G+C]−0.63 (% formamide).

In a preferred method of the invention said RNAi molecule is derived from a nucleic acid sequence encoding a Notch receptor processing factor polypeptide selected from the group consisting of: Nrarp; P300; presenilin associated protein; presenilin 1; presenilin 2; or Sel-1.

In an alternative preferred method of the invention said RNAi molecule is derived from a nucleic acid molecule encoding a Notch target gene selected from the group consisting of: HERP1; HERP2; HES1; HES 2; HES 4; HES6, HES7, HERP1, HERP2, HESR1, HEY1, HEY2, HEYL HRT1, HRT2, HRT3 CHF1, CHF2 GRIDLOCK.

In a preferred method of the invention said RNAi molecule is derived from a nucleic acid molecule comprising a nucleic acid sequence selected from the group consisting of:
i) a nucleic acid sequence as represented by the sequences in SEQ ID NO: 7-23, or fragment thereof;
ii) a nucleic acid sequence which hybridises to the nucleic acid sequences of SEQ ID NO: 7-23 and is a Notch-signalling target gene;
iii) a nucleic acid sequence which comprise sequences which are degenerate as a result of the genetic code to the nucleic acid sequences defined in (i) and (ii).

In a further alternative preferred method of the invention said RNAi molecule is derived from a nucleic acid molecule encoding a Wnt ligand processing factor selected from the group consisting of: LRP1, LRP2; LRP3; LRP4; LRP5; LRP6; LRP8; or Porcupine.

Alternatively said RNAi molecule is derived from a nucleic acid molecule encoding an extracellular Wnt antagonist selected from the group consisting of: Dkk1; Dkk2; Dkk3; Dkk4; Frzb; or SARP1.

In a further preferred method of the invention said RNAi molecule is derived from a nucleic acid molecule encoding a Wnt cytoplasmic acting component selected from the group consisting of: APC; Axin1; Axin2; FRAT1; GSK3; ICAT; IDAX; Par 1; or TAB1.

Alternatively, said RNAi molecule is derived from a nucleic acid molecule encoding a Wnt nuclear acting component selected from the group consisting of: β-catenin; β-TRCP; CBP; CTBP1; HBP-1; Lef1; NLK, Pontin 52; Reptin 52.

In a yet further alternative method of the invention said RNAi molecule is derived from a nucleic acid molecule which encodes a Wnt target gene selected from ASCL 1 or ASCL 2.

In a yet still further preferred method of the invention said RNAi molecule is derived from a nucleic acid molecule selected from the group consisting of: FGF 5; msx 1; neurogenin 1; neurogenin 2; neurogenin 3; or PTEN.

In an alternative preferred method of the invention said RNAi molecule is derived from a gene which encodes a polypeptide involved in modifying chromatin conformation.

In a preferred method of the invention said RNAi molecule is derived from a nucleic acid sequence which encodes a polypeptide which modifies a histone polypeptide. Preferably said histone modifying polypeptide is a histone deacetylase. Preferably said RNAi is derived from a mammalian class I histone deacetylase.

In a preferred method of the invention said nucleic acid molecule comprises a nucleic acid sequence selected from the group consisting of:
i) a nucleic acid sequence as represented by the sequences in Table 4, or fragment thereof;
ii) a nucleic acid sequence which hybridises to the nucleic acid sequences of Table 4 and which has histone deacetylase activity,
iii) a nucleic acid sequence which comprise sequences which are degenerate as a result of the genetic code to the nucleic acid sequences defined in (i) and (ii).

Preferably said histone deacetylase is selected from the group consisting of: HDAC1; HDAC2; HDAC3; HDAC 4; HDAC5; HDAC6; HDAC7; HDAC8; hSIRT2; hSIRT3; hSIRT4; hSIRT5; hSIRT6; hSIRT7; MECP2; ZNF145; TFDP1; SAP30; SAP18; RBBP7; RBBP4; RB1; MEN1.

Alternatively, said histone modifying polypeptide is a histone acetyltransferase selected from the group consisting of:
i) a nucleic acid sequence as represented by the sequences in Table 5, or fragment thereof;
ii) a nucleic acid sequence which hybridises to the nucleic acid sequences of Table 5 and which has histone acetyltransferase activity,
iii) a nucleic acid sequence which comprise sequences which are degenerate as a result of the genetic code to the nucleic acid sequences defined in (i) and (ii).

In a preferred method of the invention said histone acetyltransferase is selected from the group consisting of: Gcn 5; Gcn5L2; PCAF; MOZ; HBO; CBP; SCR-1; pGRIP; ATF-2; and HAT1.

In a further preferred method of the invention said RNAi molecule comprises a nucleic acid sequence derived from a gene selected from the group consisting of:
i) a nucleic acid sequence as represented by the sequences in Table 2, or fragment thereof;
ii) a nucleic acid sequence which hybridises to the nucleic acid sequences of Table 2 and which mediates chromatin conformation;
iii) a nucleic acid sequence which comprise sequences which are degenerate as a result of the genetic code to the nucleic acid sequences defined in (i) and (ii).

In a preferred method of the invention said nucleic acid encodes a polypeptide which mediates chromatin conformation selected from the group consisting of: EED; YY1; CBX1; CBX6; HPC2(CBX4); HPC3(CBX8); PHF1; PHF2; HPH1; HPH2; SSX1; and SSX2.

In a further preferred method of the invention said RNAi molecule comprises a nucleic acid sequence derived from a gene selected from the group consisting of:
i) a nucleic acid sequence as represented by the sequences in Table 3, or fragment thereof;
ii) a nucleic acid sequence which hybridises to the nucleic acid sequences of Table 3 and which mediates chromatin conformation;
iii) a nucleic acid sequence which comprise sequences which are degenerate as a result of the genetic code to the nucleic acid sequences defined in (i) and (ii).

In a preferred method of the invention said nucleic acid encodes a polypeptide which mediates chromatin conformation selected from the group consisting of: EPC1; EZH1; EZH2; BMI1; MEL18; SCML1; SCML2; RING1; RYBP; MLL; MLLT1; MLLT7; MLLT6; MLLT4; MLLT3; MLLT2; MLLT10; and MLL2.

In a further preferred method of the invention said RNAi molecule comprises a nucleic acid sequence derived from a gene selected from the group consisting of:
i) a nucleic acid sequence as represented by the sequences in Table 6, or fragment thereof;
ii) a nucleic acid sequence which hybridises to the nucleic acid sequences of Table 6 and which mediates chromatin conformation;
iii) a nucleic acid sequence which comprise sequences which are degenerate as a result of the genetic code to the nucleic acid sequences defined in (i) and (ii).

In a preferred method of the invention said nucleic acid encodes a polypeptide which mediates chromatin conformation selected from the group consisting of: SMARCA 5; SMARCA 2; SMARCA 4; SMARCA 3; SMARCAL1; SMARCA 1; and CHRAC1.

In a preferred method of the invention said RNAi molecule comprises a first part linked to a second part wherein said first and second parts are complementary over at least part of their length and further wherein said first and second parts form a double stranded region by complementary base pairing over at least part of their length.

The provision of first and second sequences which are complementary to one another and which comprise at least part of the coding sequence of a gene involved in stem cell differentiation means that when the sequence is transcribed into RNA the complementarity between first and second sequences allows base pairing between first and second sequences to form a double stranded RNA structure. The optional provision of a linking region between first and second parts results in the formation of a so called “hair-pin” loop structure. The transcription of the nucleic acid provides many copies of the hair-pin loop RNA which effectively functions as a RNAi molecule. The hair-pin loop RNA can be transcribed in vitro using, for example commercially available transcription kits which utilise phage RNA polymerase or in vivo using vectors adapted for expression by a cell, typically a eukaryotic cell, preferably a lineage restricted stem cell or embryonic stem cell.

According to a further aspect of the invention there is provided an RNAi molecule which comprises a sequence of a gene wherein said gene mediates stem cell differentiation.

In a preferred embodiment of the invention said RNAi molecule comprises a first part linked to a second part wherein said first and second parts are complementary over at least part of their length and further wherein said first and second parts form a double stranded region by complementary base pairing over at least part of their length.

In a further preferred embodiment of the invention said first and second parts are linked by at least one nucleotide base. In a further preferred embodiment of the invention said first and second parts are linked by 2, 3, 4, 5, 6, 7, 8, 9, or 10 nucleotide bases. In a yet further preferred embodiment of the invention said linker is at least 10 nucleotide bases.

In a further preferred embodiment said coding sequence is an exon.

Alternatively said RNA molecule is derived from intronic sequences or the 5′ and/or 3′ non-coding sequences which flank coding/exon sequences of genes which modulate stem cell differentiation.

In a further preferred embodiment of the invention the length of the RNAi molecule is between 10 nucleotide bases (nb)-1000 nb. More preferably still the length of the RNA molecule is selected from 10 nb; 20 nb; 30 nb; 40 nb; 50 nb; 60 nb; 70 nb; 80 nb; 90 nb. More preferably still said RNA molecule is 21 nb in length. Preferably said RNAi molecule comprises 19 complementary bases with a 3′ 2 nb overhang at either end.

In a further preferred embodiment of the invention said RNA molecule is 100 nb; 200 nb; 300 nb; 400 nb; 500 nb; 600 nb; 700 nb; 800 nb; 900 nb; or 1000 nb. More preferably still said RNA molecule is at least 1000 nb.

In yet a further preferred embodiment of the invention said RNAi molecules comprise modified nucleotide bases.

It will be apparent to one skilled in the art that the inclusion of modified bases, as well as the naturally occurring bases cytosine, uracil, adenosine and guanosine, may confer advantageous properties on RNAi molecules containing said modified bases. For example, modified bases may increase the stability of the RNAi molecule thereby reducing the amount required to produce a desired effect. The provision of modified bases may also provide RNAi molecules which are more or less stable.

The term “modified nucleotide base” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-allyl; 2-O-allyl; 2′-S-alkyl; 2′-S-allyl; 2′-fluoro-; 2′-halo or 2;azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include by example and not by way of limitation; alkylated purines and/or pyrimidines; acylated purines and/or pyrimidines; or other heterocycles. These classes of pyrimidines and purines are known in the art and include, pseudoisocytosine; N4,N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2 methylthio-N6-isopentenyladenine; uracil-5-oxyacetic acid methyl ester, psueouracil; 2-thiocytosine; 5-methyl-2 thiouracil, 2-thiouracil; 4-thiouracil; 5-methyluracil; N-uracil-5-oxyacetic acid methylester, uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine;

The RNAi molecules of the invention can be synthesized using conventional phosphodiester linked nucleotides and synthesized using standard solid or solution phase synthesis techniques which are known in the art. Linkages between nucleotides may use alternative linking molecules. For example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—.

According to a further aspect of the invention there is provided a nucleic acid molecule encoding at least part of a gene which modulates stem cell differentiation comprising a first part linked to a second part which first and second parts are complementary over at least part of their length, wherein said nucleic acid molecule is operably linked to at least one further nucleic acid molecule capable of promoting transcription of said nucleic acid linked thereto and further wherein said first and second parts form a double stranded region by complementary base pairing over at least part of their length as or when said nucleic acid molecule is transcribed.

In a preferred embodiment of the invention said first and second parts are linked by linking nucleotides as hereinbefore described.

It will be apparent to one skilled in the art that the synthesis of RNA molecules which form RNA stem loops can be achieved by providing vectors which include target genes, or fragments of target genes, operably linked to promoter sequences. Typically, promoter sequences are phage RNA polymerase promoters (e.g. T7, T3, SP6). Advantageously vectors are provided with multiple cloning sites into which genes or gene fragments can be subcloned. Typically, vectors are engineered so that phage promoters flank multiple cloning sites containing the gene of interest.

Alternatively target genes or fragments of target genes can be fused directly to phage promoters by creating chimeric promoter/gene fusions via oligo synthesising technology. Constructs thus created can be easily amplified by polymerase chain reaction to provide templates for the manufacture of RNA molecules comprising stem loop RNA's.

According to a further aspect of the invention there is provided an expression vector including an expression cassette comprising at least one nucleic acid molecule encoding an RNAi molecule according to the invention.

Vectors including expression cassettes encoding stem-loop RNA's are adapted for eukaryotic gene expression. Typically said adaptation includes, by example and not by way of limitation, the provision of transcription control sequences (promoter sequences) which mediate cell/tissue specific expression. These promoter sequences may be cell/tissue specific, inducible or constitutive.

Promoter elements typically also include so called TATA box and RNA polymerase initiation selection sequences which function to select a site of transcription initiation. These sequences also bind polypeptides which function, inter alia, to facilitate transcription initiation selection by RNA polymerase.

Adaptations also include the provision of selectable markers and autonomous replication sequences which both facilitate the maintenance of said vector in either the eukaryotic cell or prokaryotic host. Vectors which are maintained autonomously are referred to as episomal vectors. Further adaptations which facilitate the expression of vector encoded genes include the provision of transcription termination sequences.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

In a preferred embodiment of the invention said RNAi molecule is derived from a nucleic acid molecule encoding a notch receptor processing factor polypeptide selected from the group consisting of: Nrarp; P300; presenilin associated protein; presenilin 1; presenilin 2; or Sel-1.

In an alternative preferred embodiment said RNAi molecule is derived from a nucleic acid molecule encoding a Notch target gene selected from the group consisting of: HERP1; HERP2; HES1; HES 2; HES 4; HES6, HES7, HERP1, HERP2, HESR1, HEY1, HEY2, HEYL HRT1, HRT2, HRT3 CHF1, CHF2 GRIDLOCK.

In a preferred embodiment of the invention said RNAi molecule is derived from a nucleic acid molecule comprising a nucleic acid sequence selected from the group consisting of:
i) a nucleic acid sequence as represented by the sequences in SEQ ID NO: 7-23, or fragment thereof;
ii) a nucleic acid sequence which hybridises to the nucleic acid sequences of SEQ ID NO: 7-23 and is a Notch signalling target gene;
iii) a nucleic acid sequence which comprise sequences which are degenerate as a result of the genetic code to the nucleic acid sequences defined in (i) and (ii).

In a further alternative method of the invention said RNAi molecule is derived from a nucleic acid molecule encoding a Wnt ligand processing factor selected from the group consisting of: LRP1 LRP2; LRP3; LRP4; LRP5; LRP6; LRP8; or Porcupine.

In a yet further alternative method said RNAi molecule is derived from a nucleic acid molecule encoding an extracellular Wnt antagonist selected from the group consisting of: Dkk1; Dkk2; Dkk3; Dkk4; Frzb; or SARP1.

In a fiber preferred method of the invention said RNAi molecule is derived from a nucleic acid molecule encoding a Wnt cytoplasmic acting component selected from the group consisting of: APC; Axin1; Axin2; FRAT1; GSK3; ICAT; IDAX; Par 1; or TAB1.

Alternatively, said RNAi molecule is derived from a nucleic acid molecule encoding a Wnt nuclear acting component selected from the group consisting of: β-catenin; β-TRCP; CBP; CTBP1; HBP-1; Lef1; NLK; Pontin 52; or Reptin 52.

In a yet further preferred method of the invention said RNAi molecule is derived from a nucleic acid molecule which encodes a Wnt target gene selected from ASCL 1 or ASCL 2.

In a yet still further preferred method of the invention said RNAi molecule is derived from the group consisting of: FGF 5; msx 1; neurogenin 1; neurogenin 2; neurogenin 3; or PTEN.

According to a further aspect of the invention there is provided a method of treatment of an animal, preferably a human, comprising administering an effective amount of at least one RNAi molecule according to the invention, to a subject to be treated.

According to a yet further aspect of the invention them is provided a method of treatment of an animal, preferably a human, comprising administering an effective amount of at least one vector which includes an RNAi molecule according to the invention, to a subject to be treated.

An effective amount is an amount sufficient to induce the differentiation of at least one stem cell into at least one lineage restricted stem cell or differentiated stem cell.

According to a further aspect of the invention there is provided a lineage restricted stem cell or a differentiated stem cell obtainable by the method according to the invention.

In a preferred embodiment of the invention said lineage restricted stem cell is selected from the group consisting of: haemopoietic stem cell; neural stem cell; bone stem cell; muscle stem cell; mesenchymal stem cell; trophoblastic stem cell; epithelial stem cell (derived from organs such as the skin, gastrointestinal mucosa, kidney, bladder, mammary glands, uterus, prostate and endocrine glands such as the pituitary); endodermal stem cell (derived from organs such as the liver, pancreas, lung and blood vessels).

In a further preferred embodiment of the invention said cell is selected from the group consisting of: a nerve cell; a mesenchymal cell; a muscle cell (cardiomyocyte); a liver cell; a kidney cell; a blood cell (e.g. erythrocyte, CD4+ lymphocyte, CD8+ lymphocyte; pancreatic β cell; epithelial cell (e.g. lung, gastric); an endothelial cell.

According to a yet further aspect of the invention there is provided a cell culture comprising at least one lineage restricted stem cell or differentiated cell according to the invention.

According to a further aspect of the invention there is provided an organ comprising a lineage restricted stem cell or a differentiated stem cell according to the invention.

According to a yet further aspect of the invention there is provided a method of treatment of an animal, preferably a human, comprising administering a cell or organ according to the invention.

An embodiment of the invention will now be described by example only and with reference to the following figures and tables wherein:

Table 1 represents the nucleic acid sequences of Notch/Wnt target genes molecules from which RNAi molecules are derived;

Table 2 represents nucleic acid sequences of polycomb target genes from which RNAi molecules are derived;

Table 3 represents nucleic acid sequences of enhancers of trithorax and polycomb target genes from which RNAi molecules are derived;

Table 4 represents nucleic acid sequences of histone deacetylase target genes from which RNAi molecules are derived;

Table 5 represents nucleic acid sequences of histone acetylase target genes from which RNAi molecules are derived;

Table 6 represents nucleic acid sequences of ATP dependent chromatin modification target genes from which RNAi molecules are derived;

Table 7 represents a selection of antibodies used to monitor stem cell differentiation;

Table 8 represents nucleic acid probes used to assess mRNA markers of stem differentiation;

Table 9 represents protein markers of stem cell differentiation;

FIG. 1 illustrates stem cell differentiation is controlled by positive and negative regulators (A). The specific cell phenotypes that are derived are a direct result of positive and negative regulators which activate or suppress particular differentiation events. RNAi can be used to control both the initial differentiation of stem cells (A) and the ultimate fate of the differentiated cells D1 and D2 by repression of positive activators which would normally promote a particular cell fate;

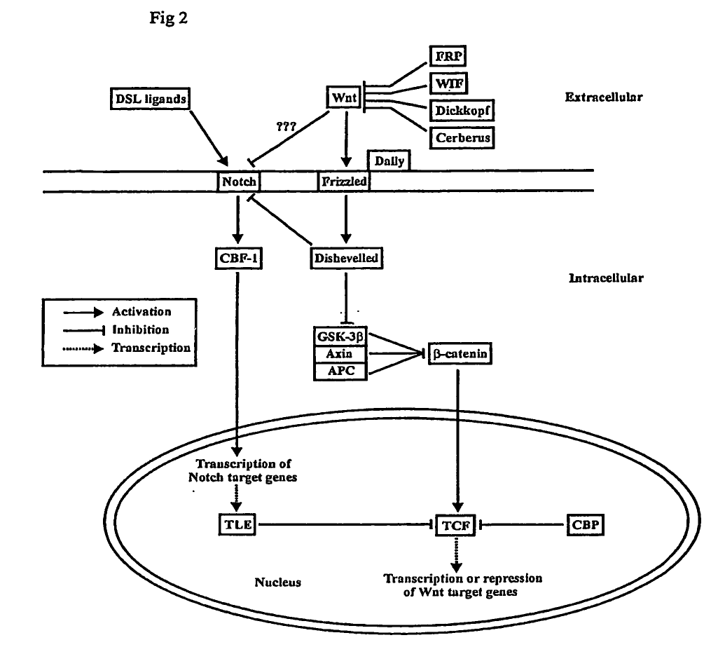

FIG. 2 represents (A) a schematic diagram illustrating the Notch and Wnt signaling pathways. The Notch and Wnt signaling pathways are shown.

Materials and Methods

Cell Culture

NTERA2 and 2102Ep human EC cell lines were maintained at high cell density as previously described (Andrews et al 1982, 1984b), in DMEM (high glucose formulation) (DMEM) (GIBCO BRL), supplemented with 10% v/v bovine foetal calf serum (GIBCO BRL), under a humidified atmosphere with 10% CO₂ in air.

Double Stranded RNA Synthesis

PCR primers were designed against the mRNA sequence of interest to give a product size of around 500 bp. At the 5′ end of each primer was added a T7 RNA polymerase promoter, comprising one or other of the following sequences: TAATACGACTCACTATAGGG; AATTATAATACGACTCACTATA. PCR was performed using these primers on an appropriate cDNA source (e.g. derived from the cell type to be targeted) and the product cloned and sequenced to confirm its identity. Using the sequenced clone as a template, further PCRs were performed as required to generate template DNA for RNA synthesis. In each case, a quantity of the PCR was electrophoresed through agarose to verify product size and abundance, whilst the remainder was purified by alkaline phenol/chloroform extraction. RNA was synthesized using the Megascript kit (Ambion Inc.) according to the manufacturer's protocol and acid phenol/chloroform extracted The simultaneous synthesis of complementary strands of RNA in a single reaction circumvents the requirement for an annealing step. However, the quality and duplexing of the synthesized RNA was confirmed by agarose gel electrophoresis, with the desired products migrating as expected for double stranded DNA of the same length.

Treatment of Human Cells with dsRNA to Produce RNAi

The following method describes RNAi of cells cultured in 6 well plates. Volumes and cell numbers should be scaled appropriately for larger or smaller culture vessels.

Cells were seeded at 500,000 per well on the day prior to treatment and grown in their normal medium. For each well to be treated, 9.5 μg of the double stranded RNA of interest was diluted in 300 μl of 150 mM NaCl. 21 μl of ExGen 500 (MBI Fermentas) was added to the diluted RNA solution and mixed by vortexing. The dsRNA/ExGen 500 mixture was incubated at room temperature for 10 minutes. 3 ml of fresh cell growth medium was then added, producing the RNAi treatment medium. Growth medium was aspirated from the culture vessel and replaced with 3 ml of RNAi treatment medium per well. Culture vessels were then centrifuged at 280 g for 5 minutes and returned to the incubator. After 12-18 hrs, RNAi treatment medium was replaced with normal growth medium and the cells maintained as required.

Total RNA Production

Growing cultures of cells were aspirated to remove the DME and foetal calf serum. Trace amounts of foetal calf serum was removed by washing in Phosphate-buffered saline. Fresh PBS was added to the cells and the cells were dislodged from the culture vessel using acid washed glass beads. The resulting cell suspension was centrifuged at 300×g. The pellets had the PBS aspirated from them. Tri reagent (Sigma, USA) was added at 1 ml per 10⁷ cells and allowed to stand for 10 mins at room temperature. The lysate from this reaction was centrifuged at 12000×g for 15 minutes at 4° C. The resulting aqueous phase was transferred to a fresh vessel and 0.5 ml of isopropanol/ml of trizol was added to precipitate the RNA. The RNA was pelleted by centrifugation at 12000×g for 10 mins at 4° C. The supernatant was removed and the pellet washed in 70% ethanol. The washed RNA was dissolved in DEPC treated double-distilled water.

Analysis of the Differentiation of EC Stem Cells Induced by Exposure to RNAi

Following exposure to RNAi corresponding to specific key regulatory genes, the subsequent differentiation of the EC cells was monitored in a variety of ways. One approach was to monitor the disappearance of typical markers of the stem cell phenotype; the other was to monitor the appearance of markers pertinent to the specific lineages induced. The relevant markers included surface antigens, mRNA species and specific proteins.

Analysis of Transfectants by Antibody Staining and FACS

Cells were treated with trypsin (0.25% v/v) for 5 mins to disaggregate the cells; they were washed and re-suspended to 2×10⁵ cells/ml. This cell suspension was incubated with 50 μl of primary antibody in a 96 well plate on a rotary shaker for 1 hour at 4° C. Supernatant from a myeloma cell line P3X63Ag8, was used as a negative control. The 96 well plate was centrifuged at 100 rpm for 3 minutes. The plate was washed 3 times with PBS containing 5% foetal calf serum to remove unbound antibody. Cell were then incubated with 50 μl of an appropriate FITC-conjugated secondary antibody at 4° C. for 1 hour. Cells were washed 3 times in PBS+5% foetal calf serum and analysed using an EPICS elite ESP flow cytometer (Coulter electronics, U.K.). (Andrews et al., 1982)

Northern Blot Analysis of RNA

RNA separation relies on the generally the same principles as standard DNA but with some concessions to the tendency of RNA to hybridise with itself or other RNA molecules. Formaldehyde is used in the gel matrix to react with the amine groups of the RNA and form Schiff bases. Purified RNA is run out using standard agarose gel electrophoresis. For most RNA a 1% agarose gel is sufficient. The agarose is made in 1×MOPS buffer and supplemented with 0.66M formaldehyde. Dryed down RNA samples are reconstituted and denatured in RNA loading buffer and loaded into the gel. Gels are run out for approx. 3 hrs (until the dye front is ¾ of the way down the gel).

The major problem with obtaining clean blotting using RNA is the presence of formaldehyde. The run out gel was soaked in distilled water for 20 mins with 4 changes, to remove the formaldehyde from the matrix. The transfer assembly was assembled in exactly the same fashion as for DNA (Southern) blotting. The transfer buffer used however was 10×SSPE. Gels were transfered overnight. The membrane was soaked in 2×SSPE to remove any agarose from the transfer assembly and the RNA was fixed to the membrane. Fixation was acheived using short-wave (254 nM) UV light. The fixed membrane was baked for 1-2 hrs to drive off any residual formaldehyde.

Hybridisation was acheived in aqueous phase with formamide to lower the hybridisation temperatures for a given probe, RNA blots were prehybridised for 2-4 hrs in northern prehybridisation solution. Labelled DNA probes were denatured at 95° C. for 5 mins and added to the blots. All hybridisation steps were carried out in rolling bottles in incubation ovens. Probes were hybridised overnight for at least 16 hrs in the prehybridisation solution A standard set of wash solutions were used. Stringency of washing was acheived by the use of lower salt containing wash buffers. The following wash procedure is outlined as follows

| | | |
|---|---|---|
| 2X SSPE | 15 mins | room temp |
| 2X SSPE | 15 mins | room temp |
| 2X SSPE/0.1% SDS | 45 mins | 65° C. |
| 2X SSPE/0.1% SDS | 45 mins | 65° C. |
| 0.1X SSPE | 15 mins | room temp |

Preparation of Radiolabelled DNA Probes

The method of Feinberg and Vogelstein (Feinberg and Vogelstein, 1983) was used to radioactively label DNA Briefly, the protocol uses random sequence hexanucleotides to prime DNA synthesis at numerous sites on a denatured DNA template using the Klenow DNA polymerase I fragment Pre-formed kits were used to aid consistency. 5-100 ng DNA fragment (obtained from gel purification of PCR or restriction digests) was made up in water, denatured for 5 mins at 95° C. with the random hexamers. The mixture was quench cooled on ice and the following were added,

5 μl [α-32P] DATP 3000 Ci/mmol

1 μl of Klenow DNA polymerase (4 U)

The reaction was then incubated at 37° C. for 1 hr. Unincorporated nucleotide were removed with spin columns (Nucleon Biosciences).

Production of cDNA

The enzymatic conversion of RNA into single stranded cDNA was achieved using the 3′ to 5′ polymerase activity of recombinant Moloney-Murine Leukemia Virus (M-MLV) reverse transcriptase primed with oligo (dT) and (dN) primers. For Reverse Transcription-Polymerase Chain Reaction, single stranded cDNA was used. cDNA was synthesised from 1 μg poly (A)+ RNA or total RNA was incubated with the following

| | |
|---|---|
| 1.0 μM | oligo(dT) primer for total RNA or random hexcamers for mRNA |
| 0.5 mM | 10 mM dNTP mix |
| 1 U/μl | RNAse inhibitor (Promega) |
| 1.0 U/μl | M-MLV reverse transcriptase in manufacturers supplied buffer |
| | (Promega) |

The reaction was incubated for 2-3 hours at 42° C.

Fluorescent Automated Sequencing

To check the specificity of the PCR primers used to generate the template used in RNAi production automatic sequencing was carried out using the prism fluorescently labelled chain terminator sequencing kit (Perkin-Elmer) (Prober et al 1987). A suitable amount of template (200 ng plasmid, 100 ng PCR product), 10 μM sequencing primer (typically a 20mer with 50% G-C content) were added to 8 μl of prism pre-mix and the total reaction volume made up to 20 ul. 24 cycles of PCR (94° C. for 10 seconds, 50° C. for 10 seconds, 60° C. for 4 minutes). Following thermal cycling, products were precipitated by the addition of 2 μl of 3M sodium acetate and 50 μl of 100% ethanol. DNA was pelleted in an Eppendorf microcentrifuge at 13000 rpm, washed once in 70% ethanol and vacuum dried. Samples were analysed by the in-house sequencing Service (Krebs Institute). Dried down samples were resuspended in 4 μl of formamide loading buffer, denatured and loaded onto a ABI 373 automatic sequencer. Raw sequence was collected and analysed using the ABI prism software and the results were supplied in the form of analysed histogram traces.

Detection of Specific Protein Targets by SDS-PAGE and Western Blotting

To obtain cell lysates monolayers of cells were rinsed 3 times with ice-old PBS supplemented with 2 mM CaCl₂. Cells were incubated with 1 ml/75 cm² flask lysis buffer (1% v/v NP40, 1% v/v DOC, 0.1 mM PMSF in PBS) for 15 min at 4° C. Cell lysates were transferred to eppendorf tubes and passed through a 21 gauge needle to shear the DNA. This was followed by freeze thawing and subsequent centrifugation (30 min, 4° C., 15000 g) to remove insoluble material. Protein concentrations of the supernatants were determined using a commercial protein assay (Biorad) and were adjusted to 1.3 mg/ml. Samples were prepared for SDS-PAGE by adding 4 times Laemmli electrophoresis sample buffer and boiling for 5 rain. After electrophoresis with 16 μg of protein on a 10% polyacrylamide gel (Laemmli, 1970) the proteins were transferred to nitro-cellulose membrane with a pore size of 0.45 μm. The blots were washed with PBS and 0.05% Tween (PBS-T). Blocking of the blots occurred in 5% milk powder in PBS-T (60 min, at RT). Blots were incubated with the appropriate primary antibody. Horseradish peroxidase labelled secondary antibody was used to visualise antibody binding by ECL (Amersham, Bucks., UK). Materials used for SDS-PAGE and western blotting were obtained from Biorad (California, USA) unless stated otherwise.

TABLE 7
Antibodies used to detect stem cell differentiation

| | | | Cell | | |
|---|---|---|---|---|---|
| Anti- | | | phenotype | Changes on | |
| body | Class | Species | detected | Differentiation | Reference |
| TRA-1- | IgM | Mouse | Human EC, | ↓ | Andrews et.al., |
| 60 | | | ES cells. | differentiation | 1984a |
| TRA-1- | IgM | Mouse | Human EC, | ↓ | Andrews et. |
| 81 | | | ES cells. | differentiation | al., 1984a |
| SSEA3 | IgM | Rat | Human EC, | ↓ | Shevinsky et al |
| | | | ES cells. | differentiation | 1982, |
| | | | | | Fenderson |
| | | | | | et al 1987 |
| SSEA4 | IgG | Mouse | Human EC, | ↓ | Kannagi et al |
| | | | ES cells. | differentiation | 1983 |
| | | | | | Fenderson |
| | | | | | et al 1987 |
| A2B5 | IgM | Mouse | | ↑ | Fenderson et al |
| | | | | differentiation | 1987 |
| ME311 | IgG | Mouse | | ↑ | Fenderson et al |
| | | | | differentiation | 1987 |
| VIN-IS- | IgM | Mouse | | ↑ | Andrews et al |
| 56 | | | | differentiation | 1990 |
| VIN-IS- | IgG | Mouse | | ↑ | Andrews et al |
| 53 | | | | differentiation | 1990 |

TABLE 8
Probes used to assess mRNA markers of differentiation

| Gene | Cell Type |
|---|---|
| Synaptophysin | Neuron |
| NeuroD1 | Neuron |
| MyoD1 | Muscle |
| Collagens | Cartilage |
| Alpha-actin | Skeletal muscle |
| Smooth-muscle actin | Smooth muscle |

TABLE 9
Protein markers of differentiation, detected by Western Blot and/or
immunofluorescence.
The following antibodies were detected by the appropriate commercially
available antibodies

| Cell Type | Antigen |
|---|---|
| Neurons | Neurofilaments |
| Glial cells | GFAP |
| Epithelial cells | Cytokeratins |
| Mesenchymal cells | Vimentin |
| Muscle | Desmin |
| Muscle | Tissue specific actins |
| Connective tissue cells | Collagens |

### REFERENCES

Andrews P. W., Goodfellow P. N., Shevinsky L., Bronson D. L. and Knowles B. B. 1982. Cell surface antigens of a clonal human embryonal carcinoma cell line: Morphological and antigenic differentiation in culture. Int. J. Cancer. 29: 523-531.
Andrews P. W., Banting G. S., Damjanov L, Arnaud D. and Avner P. 1984a. Three monoclonal antibodies defining distinct differentiation antigens associated with different high molecular weight polypeptides on the surface of human embryonal carcinoma cells. Hybridoma. 3: 347-361.
Andrews P. W., Damjanov I., Simon D., Banting G., Carlin C., Dracopoli N. C. and Fogh J. 1984b. Pluripotent embryonal carcinoma clones derived from the human teratocarcinoma cell line Tera-2: Differentiation in vivo and in vitro. Lab. Invest. 50: 147-162.
Andrews P. W., Nudelman E., Hakomori S.-i. and Fenderson B. A. 1990. Different patterns of glycolipid antigens are expressed following differentiation of TERA-2 human embryonal carcinoma cells induced by retinoic acid, hexamethylene bisacetamide (HMBA) or bromodeoxyuridine (BUdR). Differentiation. 43: 131-138.
Fenderson B. A., Andrews P. W., Nudelman E., Clausen H. and Hakomori S.-i. 1987. Glycolipid core structure switching from globo- to lacto- and ganglio-series during retinoic acid-induced differentiation of TERA-2-derived human embryonal carcinoma cells. Dev. Biol. 122: 21-34.
Kannagi, R., Levery, S. B., Ishigami, F., Hakomori, S., Shevinsky, L. H., Knowles, B. B. and Solter, D. (1983) New globoseries glycosphingolipids in human teratocarcinoma reactive with the monoclonal antibody directed to a developmentally regulated antigen, stage-specific embryonic antigen 3. J. Biol. Chem. 258, 8934-8942.
Shevinsky, L. H., Knowles, B. B., Damjanov, I. and Solter, D. (1982) Monoclonal antibody to murine embryos defines a stage-specific embryonic antigen expressed on mouse embryos and human teratocarcinoma cells. Cell 30, 697-705.
Solter, D. and Knowles, B. B. (1978) Monoclonal antibody defining a stage-specific mouse embryonic antigen (SSEA-1). Proc. natl. Acad. Sci. USA 75, 5565-5569.
Recent progress in identifying genes regulating hematopoietic stem cell function and fate Craig T Jordan, Gary Van Zant Current Opinion in Cell Biology 1998, 10:716-720.
Singer M J, Selker E U. Genetic and epigenetic inactivation of repetitive sequences in Neurospora crassa: RIP, DNA methylation, and quelling. Curr Top Microbiol Immunol. 1995; 197:165-77.
Matzke M A, Matzke A J. Gene silencing in plants: relevance for genome evolution and the acquisition of genomic methylation patterns. Novartis Found Symp. 1998; 214:168-80; discussion 181-6. Review.
Stam M, de Bruin R, van Blokland R, van der Hoorn R A, Mol J N, Kooter J M. Distinct features of post-transcriptional gene silencing by antisense transgenes in single copy and inverted T-DNA repeat loci. Plant J. 2000 January; 21(1):27-42.
Montgomery M K, Xu S, Fire A. RNA as a target of double-stranded RNA-mediated genetic interference in Caenorhabditis elegans. Proc Natl Acad Sci USA. 1998 Dec. 22; 95(26): 15502-7.
Fire A, Xu S, Montgomery M K, Kostas S A, Driver S E, Mello C C. Potent and specific genetic interference by double-stranded RNA in Caenorhabditis elegans. Nature. 1998 Feb. 19; 391(6669):806-11
Kennerdell J R, Carthew R W. Heritable gene silencing in Drosophila using double-stranded RNA. Nat Biotechnol. 2000 August; 18(8):896-898.
Shi H, Djikeng A, Mark T, Wirtz E, Tschudi C, Ullu E. Genetic interference in Trypanosoma brucei by heritable and inducible double-stranded RNA. RNA. 2000 July; 6(7):1069-76.
Wianny F, Zernicka-Goetz M. Specific interference with gene function by double-stranded RNA in early mouse development. Nat Cell Biol. 2000 February; 2(2):70-5
Thomson J A, Itskovitz-Eldor J. Shapiro S S, Waknitz M A, Swiergiel J J, Marshall V S, Jones J M. Embryonic stem cell lines derived from human blastocysts. Science. 1998 Nov. 6; 282(5391):1145-7.
Thomson J A, Kalishman J, Golos T G, Durning M, Harris C P, Becker R A, Hearn J P. Isolation of a primate embryonic stem cell line. Proc Natl Acad Sci USA. 1995 Aug. 15; 92(17):7844-8.
Prober J M, Trainor G L, Dam R J, Hobbs F W, Robertson C W, Zagursky R J, Cocuzza A J, Jensen M A, Baumeister K. A system for rapid DNA sequencing with fluorescent chain-terminating dideoxynucleotides. Science. 1987 Oct. 16; 238(4825):336-41.
Feinberg A P, Vogelstein B. A technique for radiolabeling DNA restriction endonuclease fragments to high specific activity. Anal Biochem. 1983 Jul. 1; 132(1):6-13.
Mullis K B, Faloona F A. Specific synthesis of DNA in vitro via a polymerase-catalyzed chain reaction. Methods Enzymol. 1987; 155:335-50.
Scholer H R, Hatzopoulos A K, Balling R, Suzuki N, Gruss P. A family of octamer-specific proteins present during mouse embryogenesis: evidence for germline-specific expression of an Oct factor. EMBO J. 1989 September; 8(9):2543-50.
Kraft H J, Mosselman S, Smits H A, Hohenstein P, Piek E, Chen Q, Artzt K, van Zoelen E J. Oct-4 regulates alternative platelet-derived growth factor alpha receptor gene promoter in human embryonal carcinoma cells. J Biol Chem. 1996 May 31; 271(22):12873-8.
Reubinoff B E, Pera M F, Fong C Y, Trounson A, Bongso A. Embryonic stem cell lines from human blastocysts: somatic differentiation in vitro. Nat Biotechnol. 2000 April; 18(4):399-404.
Shamblott M J, Axelman J, Wang S, Bugg E M, Littlefield J W, Donovan P J, Blumenthal P D, Hus G R, Gearhart J D. Derivation of pluripotent stem cells from cultured human primordial germ cells. Proc Natl Acad Sci USA. 1998 Nov. 10; 95(23):13726-31.

Clarke D L, Johansson C B, Wilbertz J, Veress B, Nilsson E, Karlstrom H, Lendahl U, Frisen J. Generalized potential of adult neural stem cells. Science. 2000 Jun. 2; 288(5471):1660.

TABLE 1
Notch receptor processing factors:

DEFINITION AL527440 LTI_NFL003_NBC3 Homo sapiens
Nrarp 5 Prime, partial mRNA sequence.
ACCESSION AL527440

SEQ ID NO:1

Gaggcgccttgggaccgcgtgggagccgcagccgaaccgagtagggaccg
ggaccgcgcggcgccgccgtccccggccgggcccggcccccgcgagccga
gcgcgcgcccccgtcgcccacccgggcgcggctggatgcggcggggtccc
cgcggcggcgacccccggccccgagcgccggagcgccagaggcggcgtgc
ggggcccggggacgccgcgccctstbgtgcgccgaggcgcgccccgagac
agccgggggcccgcgccgcagccgccgcccgcgctgagccccggcccggc
ccgcggcccgcgcccggcggcagcntgagccaggccgagctgtccacctg
ctccgcgccgcagacgcagcgcatcttccaggaggctgtgcgcnagggca
acacgcaggagctgcagtygctgctgcagaacatgaccaactgcgagttc
aacgtgaactcgttcgggcccgagggccagacggcgctgcaccagtcggt
catcgtcggcaacctggtgctcgtgaagctgctggtcaagttcggcgccg
acatccgcctggccaaccgcgacggctggagcgcgctgcamatcgccgcg
ttcggtggccaccaggacatcgtgctctatctcatcaccaaggcgaagta
cgcggccagcgcsggtgtatgcccgccgggaccccggaccccggccctgc
gcccgcgtcgtctctgctgtaccttcccgccaactacctcggtgcgcgcm
cggctcgcaggccccgccagaaggcccgtggcaacggcgaatacggcgcg
tgcgtcmcggccccagggtc
DEFINITION Homo sapiens E1A binding protein p300
(EP300), mRNA.
ACCESSION NM_001429

SEQ ID NO:2

Ccttgtttgtgtgctaggctgggggggagagagggcgagagagagcgggc
gagagtgggcaagcaggacgccgggctgagtgctaactgcgggacgcaga
gagtgcggaggggagtcgggtcggagagaggcggcaggggccagaacagt
ggcagggggcccggggcgcacgggctgaggcgacccccagccccctcccg
tccgcacacacccccaccgcggtccagcagccgggccggcgtcgacgcta
ggggggaccattacataacccgcgccccggccgtcttctcccgccgccgc
ggcgcccgaactgagcccggggcgggcgctccagcactggccgccggcgt
ggggcgtagcagcggccgtattattatttcgcggaaaggaaggcgaggag
gggagcgccggcgcgaggaggggccgcctgcgcccgccgccggagcgggg
cctcctcggtgggctccgcgtcggcgcgggcgtgcgggcggcgctgctcg
gcccggccccctcggccctctggtccggccagctccgctcccggcgtcct
tgccgcgcctccgccggccgccgcgcgatgtgaggcggcggcgccagcct
ggctctcggctcgggcgagttctctgcggccattaggggccggtgcggcg
gcggcgcggagcgcggcggcaggaggagggttcggagggtgggggcgcag
gcccgggagggggcaccgggaggaggtgagtgtctcttgtcgcctcctcc
tctccccccttttcgcccccgcctccttgtggcgatgagaaggaggagga
cagcgccgaggaggaagaggttgatggcggcggcggagctccgagagacc
tcggctgggcaggggccggccgtggcgggccggggactgcgcctctagag
ccgcgagttctcgggaattcgccgcagcggaccggcctcggcgaatttgt
gctcttgtgccctcctccgggcttgggccaggccggcccctcgcacttgc
ccttaccttttctatcgagtccgcatccctctccagccactgcgacccgg
cgaagagaaaaaggaacttcccccaccccctcgggtgccgtcggagcccc
ccagcccacccctgggtgcggcgcggggaccccgggccgaagaagagatt
tcctgaggattctggttttcctcgcttgtatctccgaaagaattaaaaat
ggccgagaatgtggtggaaccggggccgccttcagccaagcggcctaaac
tctcatctccggccctctcggcgtccgccagcgatggcacagattttggc
tctctatttgacttggagcacgacttaccagatgaattaatcaactctac
agaattgggactaaccaatggtggtgatattaatcagcttcagacaagtc
ttggcatggtacaagatgcagcttctaaacataaacagctgtcagaattg
ctgcgatctggtagttcccctaacctcaatatgggagttggtggcccagg
tcaagtcatggccagccaggcccaacagagcagtcctggattaggtttga
taaatagcatggtcaaaagcccaatgacacaggcaggcttgacttctccc
aacatggggatgggcactagtggaccaaatcagggtcctacgcagtcaac
aggtatgatgaacagtccagtaaatcagcctgccatgggaatgaacacag
ggacgaatgcgggcatgaatcctggaatgttggctgcaggcaatggacaa
ggataatgcctaatcaagtcatgaacggttcaattggagcaggccgaggg
cgacaggatatgcagtacccaaacccaggcatgggaagtgctggcaactt
actgactgagcctcttcagcagggctctccccagatgggaggacaaacag
gattgagaggcccccagcctcttaagatgggaatgatgaacaaccccaat
ccttatggttcaccatatactcagaatcctggacagcagattggagccag
tggccttggtctccagattcagacaaaaactgtactatcaaataacttat
ctccatttgctatggacaaaaaggcagttcctggtggaggaatgcccaac
atgggtcaacagccagccccgcaggtccagcagccaggtctggtgactcc
agttgcccaagggatgggttctggagcacatacagctgatccagagaagc
gcaagctcatccagcagcagcttgttctccttttgcatgctcacaagtgc
cagcgccgggaacaggccaatggggaagtgaggcagtgcaaccttcccca
ctgtcgcacaatgaagaatgtcctaaaccacatgacactgccagtcaggc
aagtcttgccaagtggcacactgtgcatcttctcgacaaatcatttcaca
ctggaagaattgtacaagacatgattgtcctgtgtgtctccccctcaaaa
atgctggtgataagagaaatcaacagccaattttgactggagcacccgtt
ggacttggaaatcctagctctctaggggtgggtcaacagtctgcccccaa
cctaagcactgttagtcagattgatcccagctccatagaaagagcctatg
cagctcttggactaccctatcaagtaaatcagatgccgacacaaccccag
gtgcaagcaaagaaccagcagaatcagcagcctgggcagtctccccaagg
catgcggcccatgagcaacatgagtgctagtcctatgggagtaaatggag
tgtaggagttcaaacgccgagtcttctttctgactcaatgttgcattcag
ccataaattctcaaaacccaatgatgagtgaaaatgccagtgtgccctcc
ctgggtcctatgccaacagcagctcaaccatccactactggaattcggaa
acagtggcacgaagatattactcaggatcttcgaaatcatcttgttcaca
aactcgtccaagccatatttcctacgccggatcctgctgctttaaaagac
agacggatggaaaacctagttgcatatgctcggaaagttgaaggggacat
gtatgaatctgcaaacaatcgagcggaatactaccaccttctagctgaga
aaatctataagatccagaaagaactagaagaaaaacgaaggaccagacta
cagaagcagaacatgctaccaaatgctgcaggcatggttccagtttccat
gaatccagggcctaacatgggacagccgcaaccaggaatgacttctaatg
gccctctacctgacccaagtatgatccgtggcagtgtgccaaaccagatg
atgcctcgaataactccacaatctggtttgaatcaatttggccagatgag
catggcccagccccctattgtaccccggcaaacccctcctcttcagcacc
atggacagttggctcaacctggagctctcaacccgcctatgggctatggg
cctcgtatgcaacagccttccaaccagggccagttccttcctcagactca
gttcccatcacagggaatgaatgtaacaaatatccctttggctccgtcca
gcggtcaagctccagtgtctcaagcacaaatgtctagttcttcctgcccg
gtgaactctcctataatgcctccagggtctcaggggagccacattcactg
tccccagcttcctcaaccagctcttcatcagaattcaccctcgcctgtac
ctagtcgtacccccacccctcaccatactcccccaagcataggggctcag
cagccaccagcaacaacaattccagcccctgttcctacaccaccagccat
gccacctgggccacagtcccaggctctacatccccctccaaggcagacac
ctacaccaccaacaacacaacttccccaacaagtgcagccttcacttcct
gctgcaccttctgctgaccagccccagcagcagcctcgctcacagcagag
cacagcagcgtctgttcctaccccaaacgcaccgctgcttcctccgcagc
ctgcaactccactttcccagccagctgtaagcattgaaggacaggtatca
aatcctccatctactagtagcacagaagtgaattctcaggccattgctga
gaagcagccttcccaggaagtgaagatggaggccaaaatggaagtggatc
aaccagaaccagcagatacgcagccggaggatatttcagagtctaaagtg
gaagactgtaaaatggaatctaccgaaacagaagagagaagcactgagtt
aaaaactgaaataaaagaggaggaagaccagccaagtacttcagctaccc
agtcatctccggctccaggacagtcaaagaaaaagattttcaaaccagaa
gaactacgacaggcactgatgccaacattggaggcactttaccgtcagga
tccagaatcccttccctttcgtcaacctgtggaccctcagcttttaggaa
tccctgattactttgatattgtgaagagccccatggatctttctaccatt
aagaggaagttagacactggacagtatcaggagccctggcagtatgtcga
tgatatttggcttatgttcaataatgcctggttatataaccggaaaacat
cacgggtatacaaatactgctccaagctctctgaggtctttgaacaagaa
attgacccagtgatgcaaagccttggatactgttgtggcagaaagttgga
gttctctccacagacactgtgttgctacggcaaacagttgtgcacaatac
ctcgtgatgccacttattacagttaccagaacaggtatcatttctgtgag
aagtgtttcaatgagatccaaggggagagcgtttctttgggggatgaccc
ttcccagcctcaaactacaataaataaagaacaattttccaagagaaaaa
atgacacactggatcctgaactgtttgttgaatgtacagagtgcggaaga
aagatgcatcagatctgtgtccttcaccatgagatcatctggcctgctgg
attcgtctgtgatggctgtttaaagaaaagtgcacgaactaggaaagaaa
ataagttttctgctaaaaggttgccatctaccagacttggcacctttcta
gagaatcgtgtgaatgactttctgaggcgacagaatcaccctgagtcagg
agaggtcactgttagagtagttcatgcttctgacaaaaccgtggaagtaa
aaccaggcatgaaagcaaggtttgtggacagtggagagatggcagaatcc
tttccataccgaaccaaagccctctttgcctttgaagaaattgatggtgt
tgacctgtgcttctttggcatgcatgttcaagagtatggctctgactgcc
ctccacccaaccagaggagagtatacatatcttacctcgatagtgttcat
ttcttccgtcctaaatgcttgaggactgcagtctatcatgaaatcctaat
tggatatttagaatatgtcaagaaattaggttacacaacagggcatattt
gggcatgtccaccaagtgagggagatgattatatcttccattgccatcct
cctgaccagaagatacccaagcccaagcgactgcaggaatggtacaaaaa
aatgcttgacaaggctgtatcagagcgtattgtccatgactacaaggata
tttttaaacaagctactgaagatagattaacaagtgcaaaggaattgcct
tatttcgagggtgatttctggcccaatgttctggaagaaagcattaagga
actggaacaggaggaagaagagagaaaacgagaggaaaacaccagcaatg
aaagcacagatgtgaccaagggagacagcaaaaatgctaaaaagaagaat
aataagaaaaccagcaaaaataagagcagcctgagtaggggcaacaagaa
gaaacccgggatgcccaatgtatctaacgacctctcacagaaactatatg
ccaccatggagaagcataaagaggtcttctttgtgatccgcctcattgct
ggccctgctgccaactccctgcctcccattgttgatcctgatcctctcat
cccctgcgatctgatggatggtcgggatgcgtttctcacgctggcaaggg
acaagcacctggagttctcttcactccgaagagcccagtggtccaccatg
tgcatgctggtggagctgcacacgcagagccaggaccgctttgtctacac
ctgcaatgaatgcaagcaccatgtggagacacgctggcactgtactgtct
gtgaggattatgacttgtgtatcacctgctataacactaaaaaccatgac
cacaaaatggagaaactaggccttggcttagatgatgagagcaacaacca
gcaggctgcagccacccagagcccaggcgattctcgccgcctgagtatcc
agcgctgcatccagtctctggtccatgcttgccagtgtcggaatgccaat
tgctcactgccatcctgccagaagatgaagcgggttgtgcagcataccaa
gggttgcaaacggaaaaccaatggcgggtgccccatctgcaagcagctca
ttgccctctgctgctaccatgccaagcactgccaggagaacaaatgcccg
gtgccgttctgcctaaacatcaagcagaagctccggcagcaacagctgca
gcaccgactacagcaggcccaaatgcttcgcaggaggatggccagcatgc
agcggactggtgtggttgggcagcaacagggcctcccttcccccactcct
gccactccaacgacaccaactggccaacagccaaccaccccgcagacgcc
ccagcccacttctcagcctcagcctacccctcccaatagcatgccaccct
acttgcccaggactcaagctgctggccctgtgtcccagggtaaggcagca
ggccaggtgacccctccaacccctcctcagactgctcagccaccccttcc
agggcccccacctacagcagtggaaatggcaatgcagattcagagagcag
cggagacgcagcgccagatggcccacgtgcaaatttttcaaaggccaatc
caacaccagatgcccccgatgactcccatggcccccatgggtatgaaccc
acctcccatgaccagaggtcccagtgggcatttggagccagggatgggac
cgacagggatgcagcaacagccaccctggagccaaggaggattgcctcag
ccccagcaactacagtctgggatgccaaggccagccatgatgtcagtggc
ccagcatggtcaacctttgaacatggctccacaaccaggattgggccagg
taggtatcagcccactcaaaccaggcactgtgtctcaacaagccttacaa
aaccttttgcggactctcaggtctcccagctctcccctgcagcagcaaca
ggtgcttagtatccttcacgccaacccccagctgttggctgcattcatca
agcagcgggctgccaagtatgccaactctaatccacaacccatccctggg
cagcctggcatgccccaggggcagccagggctacagccacctaccatgcc
aggtcagcagggggtccactccaatccagccatgcagaacatgaatccaa
tgcaggcgggcgttcagagggctggcctgccccagcagcaaccacagcag
caactccagccacccatgggagggatgagcccccaggctcagcagatgaa
catgaaccacaacaccatgccttcacaattccgagacatcttgagacgac
agcaaatgatgcaacagcagcagcaacagggagcagggccaggaataggc
cctggaatggccaaccataaccagttccagcaaccccaaggagttggcta
cccaccacagccgcagcagcggatgcagcatcacatgcaacagatgcaac
aaggaaatatgggacagataggccagcttccccaggccttgggagcagag
gcaggtgccagtctacaggcctatcagcagcgactccttcagcaacagat
ggggtcccctgttcagcccaaccccatgagcccccagcagcatatgctcc
caaatcaggcccagtccccacacctacaaggccagcagatccctaattct
ctctccaatcaagtgcgctctccccagcctgtcccttctccacggccaca
gtcccagcccccccactccagtccttccccaaggatgcagcctcagcctt
ctccacaccacgtttccccacagacaagttccccacatcctggactggta
gctgcccaggccaaccccatggaacaagggcattttgccagcccggacca
gaattcaatgctttctcagcttgctagcaatccaggcatggcaaacctcc
atggtgcaagcgccacggacctgggactcagcaccgataactcagacttg
aattcaaacctctcacagagtacactagacatacactagagacaccttgt
attttgggagcaaaaaaattattttctcttaacaagactttttgtactga
aaacaatttttttgaatctttcgtagcctaaaagacaattttccttggaa
cacataagaactgtgcagtagccgtttgtggtttaaagcaaacatgcaag
atgaacctgagggatgatagaatacaaagaatatatttttgttatgggct
ggttaccaccagcctttcttcccctttgtgtgtgtggttcaagtgtgcac
tgggaggaggctgaggcctgtgaagccaaacaatatgctcctgccttgca
cctccaataggttttattattttttttaaattaatgaacatatgtaatat
taatgaacatatgtaatattaatagttattatttactggtgcagatggtt
gacatttttccctattttcctcactttatggaagagttaaaacatttcta
aaccagaggacaaaaggggttaatgttactttgaaattacattctatata
tatataaatatatataaatatatattaaaataccagttttttttctctgg
gtgcaaagatgttcattcttttaaaaaatgtttaaaaaaaa
DEFINITION Homo sapiens presenilin-associated
protein mRNA, complete cds.
ACCESSION AF189289

SEQ ID NO:3

Cggtgccgcggggatggcgggagccggagctggagccggagctcgcggcg
gagcggcggcgggggtcgaggctcgagctcgcgatccaccgcccgcgcac
cgcgcacatcctcgccaccctcggcctgcggctcagccctcggcccgcag
gatggatggcgggtcagggggcctggggtctggggacaacgccccgacca
ctgaggctcttttcgtggcactgggcgcgggcgtgacggcgctcagccat
cccctgctctacgtgaagctgctcatccaggtgggtcatgagccgatgcc
ccccacccttgggaccaaatgtgctggggaggaaggtcctctatctgccg
agcttcttcacctacgccaagtacatcgtgcaagtggatggtaagatagg
gctgttccgaggcctgagtccccggctgatgtccaacgccctctctactg
tgactcggggtagcatgaagaaggttttccctccagatgagattgagcag
gtttccaacaaggatgatatgaagacttccctgaagaaagttgtgaagga
gacctcctacgagatgatgatgcagtgtgtgtcccgcatgttggcccacc
ccctgcatgtcatctcaatgcgctgcatggtccagtttgtgggacgggag
gccaagtacagtggtgtgctgagctccattgggaagattttcaaagagga
agggctgctgggattcttcgttggattaatccctcacctcctgggcgatg
tggttttcttgtggggctgtaacctgctggcccacttcatcaatgcctac
ctggtggatgacagcgtgagtgacaccccaggggggctgggaaacgacca
gaatccaggttcccagttcagccaggccctggccatccggagctatacca
agttcgtgatggggattgcagtgagcatgctgacctaccccttcctgcta
gttggcgacctcatggctgtgaacaactgcgggctgcaagctgggctccc
cccttactccccagtgttcaaatcctggattcactgctggaagtacctga
gtgtgcagggccagctcttccgaggctccagcctgcttttccgccgggtg
tcatcaggatcatgctttgccctggagtaacctgaatcatctaaaaaaca
cggtctcaacctggccaccgtgggtgaggcctgaccaccttgggacacct
gcaagacgactccaacccaacaacaaccagatgtgctccagcccagccgg
gcttcagttccatatttgccatgtgtctgtccagatgtggggttgagcgg
gggtggggctgcacccagtggattgggtcacccggcagacctagggaagg
tgaggcgaggtggggagttggcagaatccccatacctcgcagatttgctg
agtctgtcttgtgcagagggccagagaatggcttatgggggcccaggttg
gatggggaaaggctaatggggtcagaccccaccccgtctacccctccagt
cagcccagcgcccatcctgcagctcagctgggagcatcattctcctgctt
tgtacatagggtgtggtcccctggcacgtggccaccatcatgtctaggcc
tatgctaggaggcaaatggccagctctgcctgtgtttttctcaacactac
ttttctgatatgagggcagcacctgcctctgaatgggaaatcatgcaact
actcagaatgtgtcctcctcatctaatgctcatctgtttaatggtgatgc
ctcgcgtacaggatctggttacctgtgcagttgtgaatacccagaggttg
ggcagatcagtgtctctagtcctacccagttttaaagttcatggtaagat
ttgacctcatctcccgcaaataaatgtattggtgatttggaaaaaaaaaa
aaaaaaaaaaaaaaaaaaa
DEFINITION Homo sapiens presenilin 1 (PSEN1) mRNA,
complete cds.
ACCESSION AF416717

SEQ ID NO:4

Atgacagagttacctgcaccgttgtcctacttccagaatgcacagatgtc
tgaggacaaccacctgagcaatactgtacgtagccagaatgacaatagag
aacggcaggagcacaacgacagacggagccttggccaccctgagccatta
tctaatggacgaccccagggtaactcccggcaggtggtggagcaagatga
ggaagaagatgaggagctgacattgaaatatggcgccaagcatgtgatca
tgctctttgtccctgtgactctctgcatggtggtggtcgtggctaccatt
aagtcagtcagcttttatacccggaaggatgggcagctaatctatacccc
attcacagaagataccgagactgtgggccagggagccctgcactcaattc
tgaatgctgccatcatgatcagtgtcattgttgtcatgactatcctcctg
gtggttctgtataaatacaggtgctataaggtcatccatgcctggcttat
tatatcatctctattgttgctgttctttttttcattcatttacttggggg
aagtgtttaaaacctataacgttgctgtggactacattactgttgcactc
ctgatctggaattttggtgtggtgggaatgatttccattcactggaaagg
tccacttcgactccagcaggcatatctcattatgattagtgccctcatgg
ccctggtgtttatcaagtacctccctgaatggactgcgtggctcatcttg
gctgtgatttcggtatatgatttagtggctgttttgtgtccgaaaggtcc
acttcgtatgctggttgaaacagctcaggagagaaatgaaacgctttttc
cagctctcatttactcctcaacaatggtgtggttggtgaatatggcagaa
ggagacccggaagctcaaaggagagtatccaaaaattccaagtataatgc
agaaagagcctgtctgcctcctgctgccatcaacctgctgtctatagctc
ccatggcacccaggctgttcatgccaaagggtgcctgcaggcccacggca
cagaaagggagtcacaagacactgttgcagagaatgatgatggcgggttc
agtgaggaatgggaagcccagagggacagtcatctagggcctcatcgctc
tacacctgagtcacgagctgctgtccaggaactttccagcagtatcctcg
ctggtgaagacccagaggaaaggggagtaaaacttggattgggagatttc
attt
DEFINITION Homo sapiens presenilin 2 (Alzheimer
disease 4) (PSEN2), transcript variant 1, mRNA.
ACCESSION NM_000447

SEQ ID NO:5

Cgagcggcggcggagcaggcatttccagcagtgaggagacagccagaagc
aagctattggagctgaaggaacctgagacagaagctagtcccccctctga
attttactgatgaagaaactgaggccacagagctaaagtgacttttccca
aggtcgcccagcgaggacgtgggacttctcagacgtcaggagagtgatgt
gagggagctgtgtgaccatagaaagtgacgtgttaaaaaccagcgctgcc
ctctttgaaagccagggagcatcattcatttagcctgctgagaagaagaa
accaagtgtccgggattcagacctctctgcggccccaagtgttcgtggtg
cttccagaggcagggctatgctcacattcatggcctctgacagcgaggaa
gaagtgtgtgatgagcggacgtccctaatgtcggccgagagccccacgcc
gcgctcctgccaggagggcaggcagggcccagaggatggagagaacactg
cccagtggagaagccaggagaacgaggaggacggtgaggaggaccctgac
cgctatgtctgtagtggggttcccgggcggccgccaggcctggaggaaga
gctgaccctcaaatacggagcgaagcacgtgatcatgctgtttgtgcctg
tcactctgtgcatgatcgtggtggtagccaccatcaagtctgtgcgcttc
tacacagagaagaatggacagctcatctacacgacattcactgaggacac
accctcggtgggccagcgcctcctcaactccgtgctgaacaccctcatca
tgatcagcgtcatcgtggttatgaccatcttcttggtggtgctctacaag
taccgctgctacaagttcatccatggctggttgatcatgtcttcactgat
gctgctgttcctcttcacctatatctaccttggggaagtgctcaagacct
acaatgtggccatggactaccccaccctcttgctgactgtctggaacttc
ggggcagtgggcatggtgtgcatccactggaagggccctctggtgctgca
gcaggcctacctcatcatgatcagtgcgctcatggccctagtgttcatca
agtacctcccagagtggtccgcgtgggtcatcctgggcgccatctctgtg
tatgatctcgtggctgtgctgtgtcccaaagggcctctgagaatgctggt
agaaactgcccaggagagaaatgagcccatattccctgccctgatatact
catctgccatggtgtggacggttggcatggcgaagctggacccctcctct
cagggtgccctccagctcccctacgacccggagatggaagaagactccta
tgacagttttggggagccttcataccccga
DEFINITION Homo sapiens F-box protein SEL10
(SEL10) mRNA, complete cds.
ACCESSION AY008274

SEQ ID NO:6

Ctcagcaggtcaggacatttggtaggggaaggttgaaagacaaaagcagc
aggccttgggttctcagccttttaaaaactattattaaatatatattttt
aaaatttagtggttagagctttagtaatgtgcctgtattacatgtagaga
gtattcgtcaaccaagaggagtttaaaatgtcaaaaccgggaaaacctac
tctaaaccatggcttggttcctgttgatcttaaaagtgcaaaagagcctc
taccacatcaaaccgtgatgaagatatttagcattagcatcattgcccaa
ggcctccctttttgtcgaagacggatgaaaagaaagttggaccatggttc
tgaggtccgctctttttctttgggaaagaaaccatgcaaagtctcagaat
atacaagtaccactgggcttgtaccatgttcagcaacaccaacaactttt
ggggacctcagagcagccaatggccaagggcaacaacgacgccgaattac
atctgtccagccacctacaggcctccaggaatggctaaaaatgtttcaga
gctggagtggaccagagaaattgcttgctttagatgaactcattgatagt
tgtgaaccaacacaagtaaaacatatgatgcaagtgatagaaccccagtt
tcaacgagacttcatttcattgctccctaaagagttggcactctatgtgc
tttcattcctggaacccaaagacctgctacaagcagctcagacatgtcgc
tactggagaattttggctgaagacaaccttctctggagagagaaatgcaa
agaagaggggattgatgaaccattgcacatcaagagaagaaaagtaataa
aaccaggtttcatacacagtccatggaaaagtgcatacatcagacagcac
agaattgatactaactggaggcgaggagaactcaaatctcctaaggtgct
gaaaggacatgatgatcatgtgatcacatgcttacagttttgtggtaacc
gaatagttagtggttctgatgacaacactttaaaagtttggtcagcagtc
acaggcaaatgtctgagaacattagtgggacatacaggtggagtatggtc
atcacaaatgagagacaacatcatcattagtggatctacagatcggacac
tcaaagtgtggaatgcagagactggagaatgtatacacaccttatatggg
catacttccactgtgcgttgtatgcatcttcatgaaaaaagagttgttag
cggttctcgagatgccactcttagggtttgggatattgagacaggccagt
gtttacatgttttgatgggtcatgttgcagcagtccgctgtgttcaatat
gatggcaggagggttgttagtggagcatatgattttatggtaaaggtgtg
ggatccagagactgaaacctgtctacacacgttgcaggggcatactaata
gagtctattcattacagtttgatggtatccatgtggtgagtggatctctt
gatacatcaatccgtgtttgggatgtggagacagggaattgcattcacac
gttaacagggcaccagtcgttaacaagtggaatggaactcaaagacaata
ttcttgtctctgggaatgcagattctacagttaaaatctgggatatcaaa
acaggacagtgtttacaaacattgcaaggtcccaacaagcatcagagtgc
tgtgacctgtttacagttcaacaagaactttgtattaccagctcagatga
tggaactgtaaaactatgggacttgaaaacgggtgaatttattcgaaacc
tagtcacattggagagtggggggagtgggggagttgtgtggcggatcaga
gcctcaaacacaaagctggtgtgtgcagttgggagtcggaatgggactga
agaaaccaagctgctggtgctggactttgatgtggacatgaagtgaagag
cagaaaagatgaatttgtccaattgtgtagacgatatactccctgccctt
ccccctgcaaaaagaaaaaaagaaaagaaaaagaaaaaaatcccttgttc
tcagtggtgcaggatgttggcttggggcaacagattgaaaagacctacag
actaagaaggaaaagaagaagagatgacaaaccataactgacaagagagg
cgtctgctgtctcatcacataaaaggcttcacttttgactgagggcagct
ttgcaaaatgagactttctaaatcaaaccaggtgcaattatttctttatt
ttcttctccagtggtcattggggcagtgttaatgctgaaacatcattaca
gattctgctagcctgttcttttaccactgacagctagacacctagaaagg
aactgcaataatatcaaaacaagtactggttgactttctaattagagagc
atctgcaacaaaaagtcatttttctggagtggaaaagcttaaaaaaatta
ctgtgaattgtttttgtacagttatcatgaaaagcttttttttttatttt
ttngccaaccattgccaatgtcaatcaatcacagtattagcctctgttaa
tctatttactgttgcttccatatacattcttcaatgcatatgttgctcaa
aggtggcaagttgtcctgggttctgtgagtcctgagatggatttaattct
tgatgctggtgctagaagtaggtcttcaaatatgggattgttgtcccaac
cctgtactgtactcccagtggccaaacttatttatgctgctaaatgaaag
aaagaaaaaagcaaattattttttttattttttttctgctgtgacgtttt
agtcccagactgaattccaaatttgctctagtttggttatggaaaaaaga
ctttttgccactgaaacttgagccatctgtgcctctaagaggctgagaat
ggaagagtttcagataataaagagtgaagtttgcctgcaagtaaagaatt
gagagtgtgtgcaaagcttattttcttttatctgggcaaaaattaaaaca
cattccttggaacagagctattacttgcctgttctgtggagaaacttttc
tttttgagggctgtggtgaatggatgaacgtacatcgtaaaactgacaaa
atattttaaaaatatataaaacacaaaattaaaataaagttgctggtcag
tcttagtgttttacagtatttgggaaaacaactgttacagttttattgct
ctgagtaactgacaaagcagaaactattcagtttttgtagtaaaggcgtc
acatgcaaacaaacaaaatgaatgaaacagtcaaatggtttgcctcattc
tccaagagccacaactcaagctgaactgtgaaagtggtttaacactgtat
cctaggcgatcttttttcctccttctgtttatttttttgnttgttttatt
tatagtctgatttaaaacaatcagattcaagttggttaattttagttatg
taacaacctgacatgatggaggaaaacaacctttaaagggattgtgtcta
tggtttgattcacttagaaattttattttcttataacttaagtgcaataa
aatgtgttttttcatgtt

Target genes (transcription factors):

DEFINITION Homo sapiens HES-related repressor
protein 1 HERP1 mRNA, complete cds.
ACCESSION AF232238

SEQ ID NO:7

Gtcgaccgcctgcccaggcccggggagggaggaggcgggcgtcagggtgc
tgcgccccgctcggcgtccgagcttccggccgggctgtgccccgcgcggt
cttcgccgggatgaagcgcccctgcgaggagacgacctccgagagcgaca
tggacgagaccatcgacgtggggagcgagaacaattactcggggcaaagt
actagctctgtgattagattgaattctccaacaacaacatctcagattat
ggcaagaaagaaaaggagagggattatagagaaaaggcgtcgggatcgga
taaataacagtttatctgagttgagaagacttgtgccaactgcttttgaa
aaacaaggatctgcaaagttagaaaaagctgaaatattgcaaatgacagt
ggatcatttgaagatgcttcaggcaacagggggtaaaggctactttgacg
cacacgctcttgccatggacttcatgagcataggattccgagagtgccta
acagaagttgcgcggtacctgagctccgtggaaggcctggactcctcgga
tccgctgcgggtgcggcttgtgtctcatctcagcacttgcgccacccagc
gggaggcggcggccatgacatcctccatggcccaccaccatcatccgctc
cacccgcatcactgggccgccgccttccaccacctgcccgcagccctgct
ccagcccaacggcctccatgcctcagagtcaaccccttgtcgcctctcca
caacttcagaagtgcctcctgcccacggctctgctctcctcacggccacg
tttgcccatgcggattcagccctccgaatgccatccacgggcagcgtcgc
cccctgcgtgccacctctctccacctctctcttgtccctctctgccaccg
tccacgccgcagccgcagcagccaccgcggctgcacacagcttccctctg
tccttcgcgggggcattccccatgcttcccccaaacgcagcagcagcagt
ggccgcggccacagccatcagcccgcccttgtcagtatcagccacgtcca
gtcctcagcagaccagcagtggaacaaacaataaaccttaccgaccctgg
gggacagaagttggagctttttaaatttttcttgaacttcttgcaatagt
aactgaatgtcctccatttcagagtcagcttaaaacctctgcaccctgaa
ggtagccatacagatgccgacagatccacaaaggaacaataaagctattt
gagacac
DEFINITION Homo sapiens HES-related repressor
protein 2 HERP2 mRNA, complete cds.
ACCESSION AF232239

SEQ ID NO:8

Tcagtgtgtgcggaacgcaagcagccgagagcggagaggcgccgctgtag
ttaactcctccctgcccgccgcgccgaccctccccaggaacccccaggga
gccagcatgaagcgagctcaccccgagtacagctcctcggacagcgagct
ggacgagaccatcgaggtggagaaggagagtgcggacgagaatggaaact
tgagttcggctctaggttccatgtccccaactacatcttcccagattttg
gccagaaaaagacggagaggaataattgagaagcgccgacgagaccggat
caataacagtttgtctgagctgagaaggctggtacccagtgcttttgaga
agcagggatctgctaagctagaaaaagccgagatcctgcagatgaccgtg
gatcacctgaaaatgctgcatacggcaggagggaaaggttactttgacgc
gcacgcccttgctatggactatcggagtttgggatttcgggaatgcctgg
cagaagttgcgcgttatctgagcatcattgaaggactagatgcctctgac
ccgcttcgagttcgactggtttcgcatctcaacaactacgcttcccagcg
ggaagccgcgagcggcgcccacgcgggcctcggacacattccctggggga
ccgtcttcggacatcacccgcacatcgcgcacccgctgttgctgccccag
aacggccacgggaacgcgggcaccacggcctcacccacggaaccgcacca
ccagggcaggctgggctcggcacatccggaggcgcctgctttgcgagcgc
cccctagcggcagcttcggaccggtgctccctgtggtcacctccgcctcc
aaactgtcgctgcctctgctctcctcagtggcctccctgtcggccttccc
cttctctttcggctccttccacttactgtctcccaatgcactgagccctt
cagcacccacgcaggctgcaaaccttggcaagccctatagaccttggggg
acggagatcggagctttttaaagaactgatgtagaatgagggaggggaaa
gtttaaaatcccagctgggctggactgttgccaacatcaccttaaagtcg
tcagtaaaagtaaaaaggaaaaaggtacactttcagataatttttttttt
aaagactaaaggtttgttggtttacttttatcttttttaatgtttttttc
atcatgtcatgtattgcagtttttaaaaactagttgttaaattttgttca
agacattaaattgaaatagtgagtataagccaacactttgtgataggttt
gtactgtgcctaatttactttgtaaaccagaatgattccgtttttgcctc
aaaatttggggaatcttaacatttaggtatttttggtctgtttttctcct
tgtatagttatggtctgtttttagaattaattttccaaaccactatgctt
aatgttaacatgattctgtttgttaatattttgacagattaaggtgttgt
ataaataatattcttttggggggaggggaactatattgaattttatattt
ctgagcaaagcgttgacaaatcagatgatcagctttatccaagaaagaag
actagtaaattgtctgcctcctatagcagaaaggtgaatgtacaaactgt
tggtggcctgaatccatctgaccagctgctggtatctgccaggactggca
gttctgatttagttaggaggaccgctgataggttaggtctcatttggagt
gttggtggaaaggaaactgaaggtaattgaatagaatacgcctgcattta
ccagccccagcaacacaaagaatttttaatcacacggatctcaaattcac
aaatgttaacatggataagtgatcatggtgtgcgagtggtcaattgagta
gtacagtggaaactgttaaatgcataacctaattttcctgggactgccat
attttcttttaactggaaatttttatgtgagttttccttttggtgcatgg
aactgtggttgccaaggtatttaaaagggctttcctgcctccttctcttt
gatttatttaatttgatttgggctataaaatatcatttttcaggtttatt
cttttagcaggtgtagttaaacgacctccactgaactgggtttgacctct
gttgtactgatgtgttgtgactaaataaaaaagaaagaacaaagtaaaaa
aaaaaaaaaaaaaaaaaaaaaa
DEFINITION Homo sapiens hairy (Drosophila)-homolog
(HRY), mRNA.
ACCESSION NM_005524

SEQ ID NO:9

Atcacacaggatccggagctggtgctgataacagcggaatcccccgtcta
cctctctccttggtcctggaacagcgctactgatcaccaagtagccacaa
aatataataaaccctcagcacttgctcagtagttttgtgaaagtctcaag
taaaagagacacaaacaaaaaattctttttcgtgaagaactccaaaaata
aaattctctagagataaaaaaaaaaaaaaaaggaaaatgccagctgatat
aatggagaaaaattcctcgtccccggtggctgctaccccagccagtgtca
acacgacaccggataaaccaaagacagcatctgagcacagaaagtcatca
aagcctattatggagaaaagacgaagagcaagaataaatgaaagtctgag
ccagctgaaaacactgattttggatgctctgaagaaagatagctcgcggc
attccaagctggagaaggcggacattctggaaatgacagtgaagcacctc
cggaacctgcagcgggcgcagatgacggctgcgctgagcacagacccaag
tgtgctggggaagtaccgagccggcttcagcgagtgcatgaacgaggtga
cccgcttcctgtccacgtgcgagggcgttaataccgaggtgcgcactcgg
ctgctcggccacctggccaactgcatgacccagatcaatgccatgaccta
ccccgggcagccgcaccccgccttgcaggcgccgccaccgcccccaccgg
gacccggcggcccccagcacgcgccgttcgcgccgccgccgccactcgtg
cccatccccgggggcgcggcgccccctcccggcggcgccccctgcaagct
gggcagccaggctggagaggcggctaaggtgtttggaggcttccaggtgg
taccggctcccgatggccagtttgctttcctcattcccaacggggccttc
gcgcacagcggccctgtcatccccgtctacaccagcaacagcggcacctc
cgtgggccccaacgcagtgtcaccttccagcggcccctcgcttacggcgg
actccatgtggaggccgtggcggaactgagggggctcaggccacccctcc
tcctaaactccccaacccacctctcttccctccggactctaaacaggaac
ttgaatactgggagagaagaggacttttttgattaagtggttactttgtg
ttttttaatttctaagaagttactttttgtagagagagctgtattaagtg
actgaccatgcactatatttgtatatattttatatgttcatattggattg
cgcctttgtattataaaagctcagatgacatttcgttttttacacgagat
ttcttttttatgtgatgccaaagatgtttgaaaatgctcttaaaatatct
tcctttggggaagtttatttgagaaaatataataaaagaaaaaagtaaag
gcaaaaaaaaaaaaaaaaaa
DEFINITION Homo sapiens hairy and enhancer of
split (Drosophila) homolog 2 (HES2), mRNA.
GenBank Acc: BG470458

SEQ ID NO:10

Gcgcggggacactcgtgcgactggggcaaggtgcccacgggcttctctcc
taatttgcctcacgtaacagttgagaccccagagggcagcaaaactgggt
tcgaattggagagccgtccaggcacagacaaattcattcatctgcccagt
gcccgctaactgcggttccaggcgccggcgacgcaattccagagctgcca
ccgcttccccgcggagcatggggctgcctcgccgggcaggggacgcggcg
gagctgcgcaagagcctgaagccgctgctggagaagcgccggcgcgcgcg
catcaaccagagcctgagccagcttaaggggctcatcctgccgctgctgg
gccgggaggatgcttctggctggcacacctggcttcccctccatgctcan
gaactgcttcctactctacatccaggctcctgagcagcccccagcttaag
cttcagcagctccagggtaaatccatctccgcccagagctgactccggat
ctgccccagtgacaataacaagataaatgccttccattctcttcgtactt
acaggatgtaatccacatatttacacagtgtttcatttgcggtaatgttt
gtatagtttcttgttccttcgtacctatctgggaagcctgtg
DEFINITION Homo sapiens bHLH factor Hes4
(LOC57801), mRNA.
ACCESSION NM_021170

SEQ ID NO:11

Atggccgcagacacgccggggaaaccgagcgcctcgccgatggcaggagc
gccggccagcgccagccggaccccagacaagccccggagcgcggccgagc
accgcaagtcctccaagccggtcatggagaagcggcgccgagcgcgtatt
aacgagagcctcgctcagctcaaaaccctcatcctggacgccctcagaaa
agagagctcccgccactcgaagctggagaaggcggacatcctggagatga
ccgtgagacacctgcggagcctgcgtcgcgtgcaggtgacggccgcgctc
agcgccgacccggccgttctgggcaagtaccgcgccggcttccacgagtg
tctggcggaggtgaaccgcttcctggccggctgcgagggcgtcccggccg
acgtgcgctcccgcctgctgggccacctggcagcctgcctgcgccagctg
ggaccctcccgccgcccggcctcgctgtccccggctgcccccgcagaggc
cccagcgcccgaggtctacgcgggccgcccgctgctgccatcgctcggcg
gccccttccctctgctcgcgccgccgctgctgccgggtctgacccgggcg
ctgcccgccgcccccagggcggggccgcagggcccgggtgggccctggag
gccgtggctgcgctga
HESR1,
>gi|5059322|gb|af151522.1|af151522 homo sapiens
hairy and enhancer of split related-1 (hesr-1)
mrna, complete cds

SEQ ID NO:12

cccaggaacccccagggagccagcatgaagcgagctcaccccgagtacag
ctcctcggacagcgagctggacgagaccatcgaggtggagaaggagagtg
cggacgagaatggaaacttgagttcggctctaggttccatgtccccaact
acatcttcccagattttggccagaaaaagacggagaggaataattgagaa
gcgccgacgagaccggatcaataacagtttgtctgagctgagaaggctgg
tacccagtgcttttgagaagcagggatctgctaagctagaaaaagccgag
atcctgcagatgaccgtggatcacctgaaaatgctgcatacggcaggagg
gaaaggttactttgacgcgcacgcccttgctatggactatcggagtttgg
gatttcgggaatgcctggcagaagttgcgcgttatctgagcatcattgaa
ggactagatgcctctgacccgcttcgagttcgactggtttcgcatctcaa
caactacgcttcccagcgggaagccgcgagcggcgcccacgcgggcctcg
gacacattccctgggggaccgtcttcggacatcacccgcacatcgcgcac
ccgctgttgctgccccagaacggccacgggaacgcgggcaccacggcctc
acccacggaaccgcaccaccagggcaggctgggctcggcacatccggagg
cgcctgctttgcgagcgccccctagcggcagcctcggaccggtgctccct
gtggtcacctccgcctccaaactgtcgccgcctctgctctcctcagtggc
ctccctgtcggccttccccttctctttcggctccttccacttactgtctc
ccaatgcactgagcccttcagcacccacgcaggctgcaaaccttggcaag
ccctatagaccttgggggacggagatcggagctttttaaagaactgatgt
agaatgagggaggggaaagtttaaaatcccagctgggctggactgttgcc
aacatcaccttaaagtcgtcagtaaaagtaaaaaggaaaaaggtacactt
tcagataatttttttttaaagactaaaggtttgttggtttacttttatct
ttttaatgtttttttcatcatgtcatgtattagcagtttttaaaaactag
ttgttaaatttgttcaagacattaaattgaaatagtgagtataagccaac
actttgtgataggtttgtactgtgcctaatttactttgtaaaccagaatg
attccgtttttgcctcaaaatttggggaatcttaacatttagtatttttg
gtctgtttttctccttgtatagttatggtctgtttttagaattaattttc
caaaccactatgcttaatgttaaacatgattctgtttgttaatattttga
cagattaaggtgttgtataaataatattcttttggggggaggggaactat
attgaattttatatttctgagcaaagcgttgacaaatcagatgatcagct
ttatccaagaaagaagactagtaaattgtctgcctcctatagcagaaagg
tgaatgtacaaactgttggtggccctgaatccatctgaccagctgctggt
atctgccaggactggcagttctgatttagttaggagagagccgctgatag
gttaggtctcatttggagtgttggtggaaaggaaactgaaggtaattgaa
tagaatacgcctgcatttaccagccccagcaacacaaagaatttttaatc
acacggatctcaaattcacaaatgttaacatggataagtgatcatggtgt
gcgagtggtcaattgagtagtacagtggaaactgttaaatgcataaccta
attttcctgggactgccatattttcttttaactggaaatttttatgtgag
ttttccttttggtgcatggaactgtggttgccaaggtatttaaaagggct
ttcctgcctccttctctttgatttatttaatttgatttgggctataaaat
atcatttttcaggtttattcttttagcaggtgtagttaaacgacctccac
tgaactgggtttgacctctgttgtactgatgtgttgtgactaaataaaaa
a
HEY 1
>gi|20149602|ref|nm_012258.2|homo sapiens hairy/
enhancer-of-split related with yrpw motif 1
(hey1), mrna

SEQ ID NO:13

tcagtgtgtgcggaacgcaagcagccgagagcggagaggcgccgctgtag
ttaactcctccctgcccgccgcgccgaccctccccaggaacccccaggga
gccagcatgaagcgagctcaccccgagtacagctcctcggacagcgagct
ggacgagaccatcgaggtggagaaggagagtgcggacgagaatggaaact
tgagttcggctctaggttccatgtccccaactacatcttcccagattttg
gccagaaaaagacggagaggaataattgagaagcgccgacgagaccggat
caataacagtttgtctgagctgagaaggctggtacccagtgcttttgaga
agcagggatctgctaagctagaaaaagccgagatcctgcagatgaccgtg
gatcacctgaaaatgctgcatacggcaggagggaaaggttactttgacgc
gcacgcccttgctatggactatcggagtttgggatttcgggaatgcctgg
cagaagttgcgcgttatctgagcatcattgaaggactagatgcctctgac
ccgcttcgagttcgactggtttcgcatctcaacaactacgcttcccagcg
ggaagccgcgagcggcgcccacgcgggcctcggacacattccctggggga
ccgtcttcggacatcacccgcacatcgcgcacccgctgttgctgccccag
aacggccacgggaacgcgggcaccacggcctcacccacggaaccgcacca
ccagggcaggctgggctcggcacatccggaggcgcctgctttgcgagcgc
cccctagcggcagcttcggaccggtgctccctgtggtcacctccgcctcc
aaactgtcgctgcctctgctctcctcagtggcctccctgtcggccttccc
cttctctttcggctccttccacttactgtctcccaatgcactgagccctt
cagcacccacgcaggctgcaaaccttggcaagccctatagaccttggggg
acggagatcggagctttttaaagaactgatgtagaatgagggaggggaaa
gtttaaaatcccagctgggctggactgttgccaacatcaccttaaagtcg
tcagtaaaagtaaaaaggaaaaaggtacactttcagataatttttttttt
aaagactaaaggtttgttggtttacttttatcttttttaatgtttttttc
atcatgtcatgtattagcagtttttaaaaactagttgttaaattttgttc
aagacattaaattgaaatagtgagtataagccaacactttgtgataggtt
tgtactgtgcctaatttactttgtaaaccagaatgattccgtttttgcct
caaaatttggggaatcttaacatttaggtatttttggtctgtttttctcc
ttgtatagttatggtctgtttttagaattaattttccaaaccactatgct
taatgttaacatgattctgtttgttaatattttgacagattaaggtgttg
tataaataatattcttttggggggaggggaactatattgaattttatatt
tctgagcaaagcgttgacaaatcagatgatcagctttatccaagaaagaa
gactagtaaattgtctgcctcctatagcagaaaggtgaatgtacaaactg
ttggtggcctgaatccatctgaccagctgctggtatctgccaggactggc
agttctgatttagttaggaggaccgctgataggttaggtctcatttggag
tgttggtggaaaggaaactgaaggtaattgaatagaatacgcctgcattt
accagccccagcaacacaaagaatttttaatcacacggatctcaaattca
caaatgttaacatggataagtgatcatggtgtgcgagtggtcaattgagt
agtacagtggaaactgttaaatgcataacctaattttcctgggactgcca
tattttcttttaactggaaatttttatgtgagttttccttttggtgcatg
gaactgtggttgccaaggtatttaaaagggctttcctgcctccttctctt
tgatttatttaatttgatttgggctataaaatatcatttttcaggtttat
tcttttagcaggtgtagttaaacgacctccactgaactgggtttgacctc
tgttgtactgatgtgttgtgactaaataaaaaagaaagaacaaagtaaaa
aaaaaaaaaaaaaaaaaaaaaaa
HEY 2
>gi|6912413|ref|nm_012259.1|homo sapiens hairy/
enhancer-of-split related with yrpw motif 2
(hey2), mrna

SEQ ID NO:14

tcggcgtccgagcttccggccgggctgtgccccgcgcggtcttcgccggg
atgaagcgcccctgcgaggagacgacctccgagagcgacatggacgagac
catcgacgtggggagcgagaacaattactcggggcaaagtactagctctg
tgattagattgaattctccaacaacaacatctcagattatggcaagaaag
aaaaggagagggattatagagaaaaggcgtcgggatcggataaataacag
tttatctgagttgagaagacttgtgccaactgcttttgaaaaacaaggat
ctgcaaagttagaaaaagctgaaatattgcaaatgacagtggatcatttg
aagatgcttcaggcaacagggggtaaaggctactttgacgcacacgctct
tgccatggacttcatgagcataggattccgagagtgcctaacagaagttg
cgcggtacctgagctccgtggaaggcctggactcctcggatccgctgcgg
gtgcggcttgtgtctcatctcagcacttgcgccacccagcgggaggcggc
ggccatgacatcctccatggcccaccaccatcatccgctccacccgcatc
actgggccgccgccttccaccacctgcccgcagccctgctccagcccaac
ggcctccatgcctcagagtcaaccccttgtcgcctctccacaacttcaga
agtgcctcctgcccacggctctgctctcctcacggccacgtttgcccatg
cggattcagccctccgaatgccatccacgggcagcgtcgccccctgcgtg
ccacctctctccacctctctcttgtccctctctgccaccgtccacgccgc
agccgcagcagccaccgcggctgcacacagcttccctctgtccttcgcgg
gggcattccccatgcttcccccaaacgcagcagcagcagtggccgcggcc
acagccatcagcccgcccttgtcagtatcagccacgtccagtcctcagca
gaccagcagtggaacaaacaataaaccttaccgaccctgggggacagaag
ttggagctttttaaatttttcttgaacttcttgcaatagtaactgaatgt
cctccatttcagagtcagcttaaaacctctgcaccctgaaggtagccata
cagatgccgacagatccacaaaggaacaataaagctatttgagacacaaa
cctcacgagtggaaatgtggtattctcttttttttctctcccttttttgt
ttggttcaaggcagctcggtaactgacatcagcaacttttgaaaacttca
cacttgttaccatttagaagtttcctggaaaatatatggaccgtaccatc
cagcagtgcatcagtatgtctgaattggggaagtaaaatgccctgactga
attctcttgagactagatgggacatacatatatagagagagagtgagaga
gtcgtgtttcgtaagtgcctgagcttaggaagttttcttctggatatata
acattgcacaagggaagacgagtgtggaggataggttaagaaaggaaagg
gacagaagtcttgcaataggctgcagacattttaataccatgccagagaa
gagtattctgctgaaaccaacaggttttactggtcaaaatgactgctgaa
aataattttcaagttgaaagatctagttttatcttagtttgccttctttg
tacagacatgccaagaggtgacatttagcagtgcattggtataagcaatt
atttcatcagttctcagattaacaagcatttctgctctgcctgcaggccc
ccaggcacttttttttttggatggctcaaaatatggtgctgctttatata
aaccttacatttatatagtgcacctatgagcagttgcctaccatgtgtcc
accagaggctatttaattcatgccaacttgaaaactctccagtttgtagg
agtttggtttaatttattcagtttcattaggactatttttatatatttat
cctcttcattttctcctaatgatgcaacatctattcttgtcaccctttgg
gagaagttacatttctggaggtgatgaagcaaggagggagcactaggaag
agaaaagctacaatttttaaagctctttgtcaagttagtgattgcatttg
atcccaaaacaagatgaatgtatgcaatgggatgtacataagttattttt
gcccatgcctaaactagtgctatgtaatggggttgtggttttgttttttt
cgatttcgtttaatgacaaaataatctcttaatatgctgaaatcaagcac
gtgagagtttttgtttaaaagataagagacacagcatgtattatgcactt
catttctctactgtgtggagaaagcaataaacattatgagaatgttaaac
gttatgcaaaattatacttttaaatatttgttttgaaattactgtaccta
gtcttttttgcattactttgtaacctttttctatgcaagagtctttacat
accactaattaaatgaagtcctttttgactatt
HEYL
>gi|19923414|ref|nm_014571.2|homo sapiens hairy/
enhancer-of-split related with yrpw motif-like
(heyl), mrna

SEQ ID NO:15

accaggcagcctgcgttcgccatgaagcgacccaaggagccgagcggctc
cgacggggagtccgacggacccatcgacgtgggccaagagggccagctga
gccagatggccaggccgctgtccacccccagctcttcgcagatgcaagcc
aggaagaaacgcagagggatcatagagaaacggcgtcgagaccgcatcaa
cagtagcctttctgaattgcgacgcttggtccccactgcctttgagaaac
agggctcttccaagctggagaaagccgaggtcttgcagatgacggtggat
cacttgaaaatgctccatgccactggtgggacaggattctttgatgcccg
agccctggcagttgacttccggagcattggttttcgggagtgcctcactg
aggtcatcaggtacctgggggtccttgaagggcccagcagccgtgcagac
cccgtccggattcgccttctctcccacctcaacagctacgcagccgagat
ggagccttcgcccacgcccactggccctttggccttccctgcctggccct
ggtctttcttccatagctgtccagggctgccagccctgagcaaccagctc
gccatcctgggaagagtgcccagccctgtcctccccggtgtctcctctcc
tgcttaccccatcccagccctccgaaccgctccccttcgcagagccacag
gcatcatcctgccagcccggaggaatgtgctgcccagtcgaggggcatct
tccacccggagggcccgccccctagagaggccagcgacccctgtgcctgt
cgcccccagcagcagggctgccaggagcagccacatcgctcccctcctgc
agtcttcctccccaacaccccctggtcctacagggtcggctgcttacgtg
gctgttcccacccccaactcatcctccccagggccagctgggaggccagc
gggagccatgctctaccactcctgggtctctgaaatcactgaaatcgggg
ctttctgagctgccccttcaccaccccgccccaaggaataaggaaggttc
ttttaccaggagcccaaaaaagggcactgccttttctgctttgcttcgtg
gactggctcatatgtgaaggcacgttctccagccatcagaggccccctcc
tcctccaacccatctctccttctcactgttatcccagcttatccacccag
ctctcctggagctgttctggtctcagaggcttggttccatttctcacctg
aacagatgagtcctgggagagaccctcagagatccgcccagacccctctc
ctgccctctgcacaccagcagcaggcatgaaccttgggtctgggaaaaag
ctttaacctgcagggcaccaggacccaaggcaggctgttccttggggcgg
tcagaccccagtcaggagcaatgactgactggctgcagccttcccacgcc
aagaggctggaacatagtgtctgcctcgcttcctggagatagtaactgag
caggggctacaaagaggtctcctgggaaccctgtctgccccttcccacct
gtccttgggccacaccatcacactgaaccacaggacagaccctttctcca
ccacagccaaggcctggagactgggggcccagcagagcctgctcccaccc
tcctcccagcagcagacacccaccctctcactgactaacaggtccctgca
cacagctggcctggtaaacccagctgggaggtttctaggcagcagcaaaa
ctctgtgacagggtgtcctcacaccaggccttggacagctctcccagaca
ggagccagggttgagcaatggagagcccagcccccacgtcttacagtcgc
catcctccaggcgtgtggtccctccccattgggtgcacagtgcagagggg
ccgtggccccatgtgatggtgcgcagagaggaacctcttgggattcagca
ccagacgtctgtgctgcctggtttgcatccggctcacagagcccagactg
ctggaacagccaaggactgtcaggctggacaaaaataactgcaaggaggg
gcaagagaaaggatgattcgaggcaccttggcccttcaaggtcatgcagt
gggtcgagcgcctgagatcctgttcaccaggactccacagagctggctct
gctcagaagccatttcattccccggctccaccctaggccactttttctaa
cagaggaaacaaatggtccagcagtcgttcccagcagaacagcggagcct
ggactgacacccagtgggaccagtgttgccacaccagttgataaaatgca
gaaacccttctgtactcgttggtaaatatctactcccccaagtgactcca
ggtgccccccaccgcctggcacttcccccaggactcctacgatctggtta
ctgcctggccgatccaaggctgtggagtcccagagccagcagttcactgg
tgctcattccacactggttagatacttcagttgtcacccctgggaagatt
ctcccacctcctccctttgatggaaccaccctccccagaggctgcattga
ggagactccacagactgaaaagtgagtttgcagaaaccttggggaaaagg
gccctttcaaagaagtggataagagggaggagatcattgagtgacccaga
aagctcttttgaaaagacagactcctcaaggagagataaagaggaaagca
cctctttcattttttagtgtgagctaattccatcagactgctgtcctcct
ggacccatctgagatgtgcagtagcaaggagaggggggatcattttagag
agtgggtcattggcagggagtgctccggagggaggcagaggggagactgt
ggtagaaggaagacagaactcacacatgctcccaggattggggacaggga
cagaggaggtaacagaaggcaaaggccagtttccccgttatcatgaaggg
gcccactcaggacaggaacaaggacaactcctcctcctcctcctcctctc
ctgctgctcctgggataccaggtcagtgatgtagtcttgcagtttggcaa
cttcctagcctgagaatccctagtggggctgtgggaaacacatttccacg
ttgcaagcatgcaactccaaagaatctgtgatgccactgaaatgagatgg
gaatgatccagctctttcagcatcttggttgaacttgctttcattgtccc
tgggatattgtggaaggaaaggtgactgtgtgatctgattctgtggtcaa
ggacttgcatcttgtgtttctatccccaagccttcctggtgtctccaact
cctaccccattgcatgggttgttgcggacatccaataaagatttttttag
tgcttctggaaaaaaaaaaaaaaaaaaaaaaaaaaaaaaaaaaaaaaaaa
aaaaaaaaaaaaaacc
HRT1

SEQ ID NO:16

atgaagcgagctcaccccgagtacagctcctcggacagcgagctggacga
gaccatcgaggtggagaaggagagtgcggacgagaatggaaacttgagtt
cggctctaggttccatgtccccaactacatcttcccagattttggccaga
aaaagacggagaggaataattgagaagcgccgacgagaccggatcaataa
cagtttgtctgagctgagaaggctggtacccagtgcttttgagaagcagg
gatctgctaagctagaaaaagccgagatcctgcagatgaccgtggatcac
ctgaaaatgctgcatacggcaggagggaaaggttactttgacgcgcacgc
ccttgctatggactatcggagtttgggatttcgggaatgcctggcagaag
ttgcgcgttatctgagcatcattgaaggactagatgcctctgacccgctt
cgagttcgactggtttcgcatctcaacaactacgcttcccagcgggaagc
cgcgagcggcgcccacgcgggcctcggacacattccctgggggaccgtct
tcggacatcacccgcacatcgcgcacccgctgttgctgccccagaacggc
cacgggaacgcgggcaccacggcctcacccacggaaccgcaccaccaggg
caggctgggctcggcacatccggaggcgcctgctttgcgagcgcccccta
gcggcagcctcggaccggtgctccctgtggtcacctccgcctccaaactg
tcgccgcctctgctctcctcagtggcctccctgtcggccttccccttctc
tttcggctccttccacttactgtctcccaatgcactgagcccttcagcac
ccacgcaggctgcaaaccttggcaagccctatagaccttgggggacggag
atcggagctttttaa
hrt2
>gi|11127955|gb|af311884.1|af311884 homo sapiens
hairy-related transcription factor 2 mrna,
complete cds

SEQ ID NO:17

atgaagcgcccctgcgaggagacgacctccgagagcgacatggacgagac
catcgacgtggggagcgagaacaattactcggggcaaagtactagctctg
tgattagattgaattctccaacaacaacatctcagattatggcaagaaag
aaaaggagagggattatagagaaaaggcgtcgggatcggataaataacag
tttatctgagttgagaagacttgtgccaactgcttttgaaaaacaaggat
ctgcaaagttagaaaaagctgaaatattgcaaatgacagtggatcatttg
aagatgcttcaggcaacagggggtaaaggctactttgacgcacacgctct
tgccatggacttcatgagcataggattccgagagtgcctaacagaagttg
cgcggtacctgagctccgtggaaggcctggactcctcggatccgctgcgg
gtgcggcttgtgtctcatctcagcacttgcgccacccagcgggaggcggc
ggccatgacatcctccatggcccaccaccatcatccgctccacccgcatc
actgggccgccgccttccaccacctgcccgcagccctgctccagcccaac
ggcctccatgcctcagagtcaaccccttgtcgcctctccacaacttcaga
agtgcctcctgcccacggctctgctctcctcacggccacgtttgcccatg
cggattcagccctccgaatgccatccacgggcagcgtcgccccctgcgtg
ccacctctctccacctctctcttgtccctctctgccaccgtccacgccgc
agccgcagcagccaccgcggctgcacacagcttccctctgtccttcgcgg
gggcattccccatgcttcccccaaacgcagcagcagcagtggccgcggcc
acagccatcagcccgcccttgtcagtatcagccacgtccagtcctcagca
gaccagcagtggaacaaacaataaaccttaccgaccctgggggacagaag
ttggagctttttaa
hrt3
>gi|11127957|gb|af311885.1|af311885 homo sapiens
hairy-related transcription factor 3 mrna,
complete cds

SEQ ID NO:18

atgaagcgacccaaggagccgagcggctccgacggggagtccgacggacc
catcgacgtgggccaagagggccagctgagccagatggccaggccgctgt
ccacccccagctcttcgcagatgcaagccaggaagaaacgcagagggatc
atagagaaacggcgtcgagaccgcatcaacagtagcctttctgaattgcg
acgcttggtccccactgcctttgagaaacagggctcttccaagctggaga
aagccgaggtcttgcagatgacggtggatcacttgaaaatgctccatgcc
actggtgggacaggattctttgatgcccgagccctggcagttgacttccg
gagcattggttttcgggagtgcctcactgaggtcatcaggtacctggggg
tccttgaagggcccagcagccgtgcagaccccgtccggattcgccttctc
tcccacctcaacagctacgcagccgagatggagccttcgcccacgcccac
tggccctttggccttccctgcctggccctggtctttcttccatagctgtc
cagggctgccagccctgagcaaccagctcgccatcctgggaagagtgccc
agccctgtcctccccggtgtctcctctcctgcttaccccatcccagccct
ccgaaccgctccccttcgcagagccacaggcatcatcctgccagcccgga
ggaatgtgctgcccagtcgaggggcatcttccacccggagggcccgcccc
ctagagaggccagcgacccctgtgcctgtcgcccccagcagcagggctgc
caggagcagccacatcgctcccctcctgcagtcttcctccccaacacccc
ctggtcctacagggtcggctgcttacgtggctgttcccacccccaactca
tcctccccagggccagctgggaggccagcgggagccatgctctaccactc
ctgggtctctgaaatcactgaaatcggggctttctga
chf1
>gi|6636408|gb|af173901.1|af173901 homo sapiens
basic helix-loop-helix factor 1 (chf1) mrna,
complete cds

SEQ ID NO:19

cggccgcgtcgacgggaaagagccgctagagcagaccgcgccgccgccgg
agccgcgcctgcccaggcccggggagggaggaggcgggcgtcagggtgct
gcgccccgctcggcgtccgagcttccggccgggctgtgccccgcgcggtc
ttcgccgggatgaagcgcccctgcgaggagacgacctccgagagcgacat
ggacgagaccatcgacgtggggagcgagaacaattactcggggcaaagta
ctagctctgtgattagattgaattctccaacaacaacatctcagattatg
gcaagaaagaaaaggagagggattatagagaaaaggcgtcgggatcggat
aaataacagtttatctgagttgagaagacttgtgccaactgcttttgaaa
aacaaggatctgcaaagttagaaaaagctgaaatattgcaaatgacagtg
gatcatttgaagatgcttcaggcaacagggggtaaaggctactttgacgc
acacgctcttgccatggacttcatgagcataggattccgagagtgcctaa
cagaagttgcgcggtacctgagctccgtggaaggcctggactcctcggat
ccgctgcgggtgcggcttgtgtctcatctcagcacttgcgccacccagcg
ggaggcggcggccatgacatcctccatggcccaccaccatcatccgctcc
acccgcatcactgggccgccgccttccaccacctgcccgcagccctgctc
cagcccaacggcctccatgcctcagagtcaaccccttgtcgcctctccac
aacttcagaagtgcctcctgcccacggctctgctctcctcacggccacgt
ttgcccatgcggattcagccctccgaatgccatccacgggcagcgtcgcc
ccctgcgtgccacctctctccacctctctcttgtccctctctgccaccgt
ccacgccgcagccgcagcagccaccgcggctgcacacagcttccctctgt
ccttcgcgggggcattccccatgcttcccccaaacgcagcagcagcagtg
gccgcggccacagccatcagcccgcccttgtcagtatcagccacgtccag
tcctcagcagaccagcagtggaacaaacaataaaccttaccgaccctggg
ggacagaagttggagctttttaaatttttcttgaacttcttgcaatagta
actgaatgtcctccatttcagagtcagcttaaaacctctgcaccctgaag
gtagccatacagatgccgacagatccacaaaggaacaataaagctatttg
agacacaaaaagtcgacgcggccgcgaattc
chf2
>gi|5802652|gb|af176422.1|af176422 homo sapiens
cardiovascular helix-loop-helix factor 2 (hcf2)
mrna, complete cds

SEQ ID NO:20

gaattcgcggccgcgtcgaccgagagcggagaggcgccgctgtagttaac
tcctccctgcccgccgcgccgaccctccccaggaacccccagggagccag
catgaagcgagctcaccccgagtacagctcctcggacagcgagctggacg
agaccatcgaggtggagaaggagagtgcggacgagaatggaaacttgagt
tcggctctaggttccatgtccccaactacatcttcccagattttggccag
aaaaagacggagaggaataattgagaagcgccgacgagaccggatcaata
acagtttgtctgagctgagaaggctggtacccagtgcttttgagaagcag
ggatctgctaagctagaaaaagccgagatcctgcagatgaccgtggatca
cctgaaaatgctgcatacggcaggagggaaaggttactttgacgcgcacg
cccttgctatggactatcggagtttgggatttcgggaatgcctggcagaa
gttgcgcgttatctgagcatcattgaaggactagatgcctctgacccgct
tcgagttcgactggtttcgcatctcaacaactacgcttcccagcgggaag
ccgcgagcggcgcccacgcgggcctcggacacattccctgggggaccgtc
ttcggacatcacccgcacatcgcgcacccgctgttgctgccccagaacgg
ccacgggaacgcgggcaccacggcctcacccacggaaccgcaccaccagg
gcaggctgggctcggcacatccggaggcgcctgctttgcgagcgccccct
agcggcagcctcggaccggtgctccctgtggtcacctccgcctccaaact
gtcgccgcctctgctctcctcagtggcctccctgtcggccttccccttct
ctttcggctccttccacttactgtctcccaatgcactgagcccttcagca
cccacgcaggctgcaaaccttggcaagccctatagaccttgggggacgga
gatcggagctttttaaagaactgatgtagaatgagggaggggaaagttta
aaatccagctgggctggactgttgccaacatcaccttaaagtcgtcagta
aaagtaaaaaggaaaaaggtacactttcagataattttttttttaaagac
taaaggtttgttggtttacttttttcttttttaatgtttttttcatcatg
tcatgtattagcagtttttaaaaaactagttgttaaattttgttcaagac
attaaattgaaatagtgagtataagccaacactttgtgataggtttgtac
tgtgcctaatttactttgtaaaccagaatgattccgtttttgcctcaaaa
tttggggaatcttaacatttagtatttttggtctgtttttctccttgtat
agttatggtctgtttttagaattaattttccaaaccactatgcttaatgt
taacatgattctgtttgttaatattttgacagattaaggtgttgtataaa
taatattcttttggggggaggggaactatattgaattttatatttctgag
caaagcgttgacaaatcagatgatcagctttatccaagaaagaagactag
taaattgtctgcctcctatagcagaaaggtgaatgtacaaactgttggtg
gccctgaatccatctgaccagctgctggtatctgccaggactggcagttc
tgatttagttaggagagagccgctgataggttaggtctcatttggagtgt
tggtggaaaggaaactgaaggtaattgaatagaatacgcctgcatttacc
agccccagcaacacaaagaatttttaatcacacggatctcaaattcacaa
atgttaacatggataagtgatcatggtgtgcgagtggtcaattgagtagt
acagtggaacctgttaaatgcataacctaattttcctgggactgccatat
tttcttttaactggaaatttttatgtgagttttccttttggtgcatggaa
ctgtggttgccaaggtatttaaaagggctttcctgcctccttctctttga
tttatttaatttgatttgggctataaaatatcatttttcaggtttattct
tttagcaggtgtagttaaacgacctccactgaactgggtttgacctctgt
tgtactgatgtgttgtgactaaataaaaaagaaagaacaaagaaaaaaaa
aaaaaaaaaaaaaaaaaaaaaaaaaaaaaaaaaa
gridlock
>gi|7274451|gb|af237949.1|af237949 homo sapiens
gridlock mrna, complete cds

SEQ ID NO:21

agggtgctgcgccccgctcggcgtccgagcttccggccgggctgtgcccc
gcgcggtcttcgccgggatgaagcgcccctgcgaggagacgacctccgag
agcgacatggacgagaccatcgacgtggggagcgagaacaattactcggg
gcaaagtactagctctgtgattagattgaattctccaacaacaacatctc
agattatggcaagaaagaaaaggagagggattatagagaaaaggcgtcgg
gatcggataaataacagtttatctgagttgagaagacttgtgccaactgc
ttttgaaaaacaaggatctgcaaagttagaaaaagctgaaatattgcaaa
tgacagtggatcatttgaagatgcttcaggcaacagggggtaaaggctac
tttgacgcacacgctcttgccatggacttcatgagcataggattccgaga
gtgcctaacagaagttgcgcggtacctgagctccgtggaaggcctggact
cctcggatccgctgcgggtgcggcttgtgtctcatctcagcacttgcgcc
acccagcgggaggcggcggccatgacatcctccatggcccaccaccatca
tccgctccacccgcatcactgggccgccgccttccaccacctgcccgcag
ccctgctccagcccaacggcctccatgcctcagagtcaaccccttgtcgc
ctctccacaacttcagaagtgcctcctgcccacggctctgctctcctcac
ggccacgtttgcccatgcggattcagccctccgaatgccatccacgggca
gcgtcgccccctgcgtgccacctctctccacctctctcttgtccctctct
gccaccgtccacgccgcagccgcagcagccaccgcggctgcacacagctt
ccctctgtccttcgcgggggcattccccatgcttcccccaaacgcagcag
cagcagtggccgcggccacagccatcagcccgcccttgtcagtatcagcc
acgtccagtcctcagcagaccagcagtggaacaaacaataaaccttaccg
accctgggggacagaagttggagctttttaaatttttcttgaacttcttg
caatagtaactgaatgtcctccatttcagagtcagcttaaaacctctgca
ccctgaaggtagccatacagatgccgacagatccacaaaggaacaataaa
gctatttgagacacaaacctcacgagtggaaatgtggtattctctttttt
ttctctcccttttttgtttggttcaaggcagctcggtaactgacatcagc
aacttttgaaaacttcacacttgttaccatttagaagtttcctggaaaat
atatggaccgtaccatccagcagtgcatcagtatgtctgaattggggaag
taaaatgccctgactgaattctcttgagactagatgggacatacatatat
agagagagagtgagagagtcgtgtttcgtaagtgcctgagcttaggaagt
tttcttctggatatataacattgcacaagggaagacgagtgtggaggata
ggttaagaaaggaaagggacagaagtcttgcaataggctgcagaca
DEFINITION Homo sapiens hairy/enhancer of split 6
(HES6) mRNA, complete cds.
ACCESSION AF260237

SEQ ID NO:22

Ctccggtccccgccgctccccgtccccgctgctcctagcccctgccgcgt
ccccggcggagcgggcatggcgccacccgcggcgcctggccgggaccgtg
tgggccgtgaggatgaggacggctgggagacgcgaggggaccgcaaggcc
cggaagcccctggtggagaagaagcggcgcgcgcggatcaacgagagcct
gcaggagctgcggctgctgctggcgggcgccgaggccaagctggagaacg
ccgaagtgctggagctgacggtgcggcgggtccagggtgtgctgcggggc
cgggcgcgcgagcgcgagcagctgcaggcggaagcgagcgagcgcttcgc
tgccggctacatccagtgcatgcacgaggtgcacacgttcgtgtccacgt
gccaggccatcgacgctaccgtcgctgccgagctcctgaaccatctgctc
gagtccatgccgctgcgtgagggcagcagcttccaggatctgctggggga
cgccctggcggggccacctagagcccctggacggagtggctggcctgcgg
ggggcgctccgggatccccaatacccagccccccgggtcctggggacgac
ctgtgctccgacctggaggaggcccctgaggctgaactgagtcaggctcc
tgctgaggggcccgacttggtgcccgcagccctgggcagcctgaccacag
cccaaattgcccggagtgtctggaggccttggtgaccaatgccagccaga
gtcctgcgggggtgggcccggccctccctggatctcctccctcctcccag
gggttcagatgtggtggggtagggccctggaagtctcccaggtcttccct
ccctcctctgatggatggcttgcagggcagcccctggtaaccagcccagt
caggccccagccccgtttcttaagaaacttttagggaccctgcagctctg
gagtgggtggagggagggagctacgggcaggaggaagaattttgtagagc
tgccagcgctctcccaaggttcacccacccaggcttcaccagccctgtgc
gggctctgggggcagaggtggcagaaatggtgctgggcactagtgttcca
ggcagccctgggctaaacaaaagcttgaacttgccacttcagcggggaga
tgagaggcaggtgcactgagctgcactgcccagagctgtgatgctctgta
catcttgtttgtagcacacttgagtttgtgtattccattgacatcaaatg
tgacaattttactaaataaagaattttggagttagtt
DEFINITION Homo sapiens bHLH factor HES7 (HES7),
mRNA.
ACCESSION NM_032580

SEQ ID NO:23

Atggtcacccgggatcgagctgagaatagggacggccccaagatgctcaa
gccgcttgtggagaagcggcgccgggaccgcatcaaccgcagcctggaag
agctgaggctgctgctgctggagcggacccgggaccagaacctccggaac
ccgaagctggagaaagcggagatattggagttcgccgtgggctacttgag
ggagcgaagccgggtggagcccccgggggttccccggtccccagtccagg
acgccaaggcgctcgccagctgctacttgtccggtttccgcgagtgcctg
cttcgcttggcggccatcgcgcacgacgccagcccgaccgcccgcgccca
gctcttctccgcgctgcacggctatctgcgccccaaaccgccccggccca
agccggtagatccgaggcctccagcgccgcgcccatccctggaccccgcc
gcaccggcccttggccctgcgctgcaccagcgccccccagtgcaccaggg
ccaccctagcccgcgctgcgcatggtccccatccctctgctccccgcgcg
ccggggattctggcgcgccggcgcccctcaccggactgctgccgccgcca
ccgccgcctcacagacaagacggggcgcccaaggccccgctgcccccgcc
gcccgctttctggagaccttggccctga
Wnt pathway
Wnt ligand processing factors:
DEFINITION Homo sapiens low density lipoprotein-
related protein 1 (alpha-2-macroglobulin
receptor) (LRP1), mRNA.
ACCESSION XM_017228

SEQ ID NO:24

Cagcggtgcgagctccaggcccatgcactgaggaggcggaaacaagggga
gcccccagagctccatcaagccccctccaaaggctcccctacccggtcca
cgccccccaccccccctccccgcctcctcccaattgtgcatttttgcagc
cggaggcggctccgagatggggctgtgagcttcgcccggggagggggaaa
gagcagcgaggagtgaagcgggggggtggggtgaagggtttggatttcgg
ggcagggggcgcacccccgtcagcaggccctccccaaggggctcggaact
ctacctcttcacccacgcccctggtgcgctttgccgaaggaaagaataag
aacagagaaggaggagggggaaaggaggaaaagggggaccccccaactgg
ggggggtgaaggagagaagtagcaggaccagaggggaaggggctgctgct
tgcatcagcccacaccatgctgaccccgccgttgctcctgctgctgcccc
tgctctcagctctggtcgcggcggctatcgacgcccctaagacttgcagc
cccaagcagtttgcctgcagagatcaaataacctgtatctcaaagggctg
gcggtgcgacggtgagagggactgcccagacggatctgacgaggcccctg
agatttgtccacagagtaaggcccagcgatgccagccaaacgagcataac
tgcctgggtactgagctgtgtgttcccatgtcccgcctctgcaatggggt
ccaggactgcatggacggctcagatgaggggccccactgccgagagctcc
aaggcaactgctctcgcctgggctgccagcaccattgtgtccccacactc
gatgggcccacctgctactgcaacagcagctttcagcttcaggcagatgg
caagacctgcaaagattttgatgagtgctcagtgtacggcacctgcagcc
agctatgcaccaacacagacggctccttcatatgtggctgtgttgaagga
tacctcctgcagccggataaccgctcctgcaaggccaagaacgagccagt
agaccggccccctgtgctgttgatagccaactcccagaacatcttggcca
cgtacctgagtggggcccaggtgtctaccatcacacctacgagcacgcgg
cagaccacagccatggacttcagctatgccaacgagaccgtatgctgggt
gcatgttggggacagtgctgctcagacgcagctcaagtgtgcccgcatgc
ctggcctaaagggcttcgtggatgagcacaccatcaaacatctccctcag
tctgcacc
DEFINITION Homo sapiens low density lipoprotein-
related protein 2 (LRP2), mRNA.
ACCESSION XM_002645

SEQ ID NO:25

Gcagacctaaaggagcgttcgctagcagaggcgctgccggtgcggtgtgc
tacgcgcgcccacctcccggggaaggaacggcgaggccggggaccgtcgc
ggagatggatcgcgggccggcagctagtggcgtgcacgctgctcctggct
ctcgtcgcctgcctagcgccggccagtggccaagaatgtgacagtgcgca
ttttcgctgtggaagtgggcattgcatccctgcagactggaggtgtgatg
ggaccaaagactgttcagatgacgcggatgaaattggctgcgctgttgtg
acctgccagcagggctatttcaagtgccagagtgagggacaatgcatccc
caactcctgggtgtgtgaccaagatcaagactgtgatgatggctcagatg
aacgtcaagattgctcacaaagtacatgctcaagtcatcagataacatgc
tccaatggtcagtgtatcccaagtgaatacaggtgcgaccacgtcagaga
ctgccccgatggagctgatgagaatgactgccagtacccaacatgtgagc
agcttacttgtgacaatggggcctgctataacaccagtcagaagtgtgat
tggaaagttgattgcagggactcctcagatgaaatcaactgcagctgaga
tatgcttgcacaatgagttttcatgtggcaatggagagtgtatccctcgt
gcttatgtctgtgaccatgacaatgattgccaagacggcagtgacgaaca
tgcttgcaactatccgacctgcggtggttaccagttcacttgccccagtg
gccgatgatttatcaaaactgggtttgtgatggagaagatgactgtaaag
ataatggagatgaagatggatgtgaaagcggtcctcatgatgttcataaa
tgttccccaagagaatggtcttgcccagagtcgggacgatgcatctccat
ttataaagtttgtgatgggattttagattgcccaggaagagaagatgaaa
acaacactagtaccggaaaatactgtagtatgactctgtgctcgccttga
actgccagtaccagtgccatgagacgccgtatggaggagcgtgtttttgt
ccccaggttatatcatcaaccacaatgacagccgtacctgtgttgagttt
gatgattgccagatatggggaatttgtgaccagaagtgtgaaagccgacc
tggccgtcacctgtgccactgtgaagaagggtatatcttggagcgtggac
agtattgcaaagctaatgattcctttggcgaggcctccattatcttctcc
aatggtcgggatttgttaattggtgatattcatggaaggagcttccggat
cctagtggagtctcagaatcgtggagtggccgtgggtgtggctttccact
atcacctgcaaagagttttttggacagacaccgtgcaaaataaggttttt
tcagttgacattaatggtttaaatatccaagaggttctcaatgtttctgt
tgaaaccccagagaacctggctgtggactgggttaataataaaatctatc
tagtggaaaccaaggtcaaccgcatagatatggtaaatttggatggaagc
tatcgggttacccttataactgaaaacttggggcatcctagaggaattgc
cgtggacccaactgttggttatttatttttctcagattgggagagccttt
ctggggaacctaagctggaaagggcattcatggatggcagcaaccgtaaa
gacttggtgaaaacaaagctgggatggcctgctggggtaactctggatat
gatatcgaagcgtgtttactgggttgactctcggtttgattacattgaaa
ctgtaacttatgatggaattcaaagggaagactgtagttcatggaggctc
cctcattcctcatccctttggagtaagcttatttgaaggtcaggtgttct
ttacagattggacaaagatggccgtgctgaaggcaaacaagttcacagag
accaaccacaagtgtactaccaggcttccctgaggccctatggagtgact
gtttaccattccctcagacagccctatgctaccaatccgtgtaaagataa
caatgggggctgtgagcaggtctgtgtcctcagccacagaacagataatg
atggtttgggtttccgttgcaagtgcacattcggcttccaactggataca
gatgagcgccactgcattgctgttcagaatttcctcattttttcatccca
agttgctattcgtgggatcccgttcaccttgtctacccaggaagatgtca
tggttccagtttcggggaatccttctttctttgtcgggattgattttgac
gcccaggacagcactatctttttttcagatatgtcaaaacacatgatttt
taagcaaaagattgatggcacaggaagagaaattctcgcagctaacaggg
tggaaaatgttgaaagtttggcttttgattggatttcaaagaatctctat
tggacagactctcattacaagagtatcagtgtcatgaggctagctgataa
aacgagacgcacagtagttcagtatttaaataacccacggtcggtggtag
ttcatccttttgccgggtatctattcttcactgattggttccgtcctgct
aaaattatgagagcatggagtgacggatctcacctcttgcctgtaataaa
cactactcttggatggcccaatggcttggccatcgattgggctgcttcac
gattgtactgggtagatgcctattttgataaaattgagcacagcaccttt
gatggtttagacagaagaagactgggccatatagagcagatgaccacatc
cgtttggacttgccatctttggagagcatttattttttactgactggaga
ctgggtgccattattcgagtcaggaaagcagatggtggagaaatgacagt
tatccgaagtggcattgcttacatactgcatttgaaatcgtatgatgtca
acatccagactggttctaacgcctgtaatcaacccacgcatcctaacggt
gactgcagccacttctgcttcccggtgccaaatttccagcgagtgtgtgg
gtgcccttatggaatgaggctggcttccaatcacttgacatgcgaggggg
acccaaccaatgaaccacccacagagcagtgtggcttattttccttcccc
tgtaaaaatggcagatgtgtgcccaattactatctctgtgatggagtcga
tgattgtcatgataacagtgatgagcaactatgtggcacacttaataata
cctgttcatcttcggcgttcacctgtggccatggggagtgcattcctgca
cactggcgctgtgacaaacgcaacgactgtgtggatggcagtgatgagca
caactgccccacccacgcacctgcttcctgccttgacacccaatacacct
gtgataatcaccagtgtatctcaaagaactgggtctgtgacacagacaat
gattgtggggatggatctgatgaaaagaactgcaattcgacagagacatg
ccaacctagtcagtttaattgccccaatcatcgatgtattgacctatcgt
ttgtctgtgatggtgacaaggattgtgttgatggatctgatgaggttggt
tgtgtattaaactgtactgcttctcaattcaagtgtgccagtggggataa
atgtattggcgtcacaaatcgttgtgatggtgtttttgattgcagtgaca
actcggatgaagcaggctgtccaaccaggcctcctggtatgtgccactca
gatgaatttcagtgccaagaagatggtatctgcatcccgaacttctggga
atgtgatgggcatccgactgcctctatggatctgatgagcacaatgcctg
tgtccccaagacttgcccttcatcatatttccactgtgacaacggaaact
gctccacagggcatggctctgtgatcgggacaatgactgcggggatatga
gtgatgagaaggactgccctactcagccctttcgctgtcctagttggcaa
ggcagtgtcttggccataacatctgtgtgaatctgagtgtagtgtgtgat
ggcatctttgactgccccaatgggacagatgagtccccactttgcaatgg
gaacagctgctcagatttcaatggtggttgtactcacgagtgtgttcaag
agccctttggggctaaatgcctatgtccattgggattcttacttgccaat
gattctaagacctgtgaagacatagatgaatgtgatattctaggctcttg
tagccagcactgttacaatatgagaggttctttccggtgctcgtgtgata
caggctacatgttagaaagtgatgggaggacttgcaaagttacagcatct
gagagtctgctgttacttgtggcaagtcagaacaaaattattgccgacag
tgtcacctcccaggtccacaatatctattcattggtcgagaatggttctt
acattgtagctgttgattttgattcaattagtggtcgtatcttttggtct
gatgcaactcagggtaaaacctggagtgcgtttcaaaatggaacggacag
aagagtggtatttgacagtagcatcatcttgactgaaactattgcaatag
attgggtaggtcgtaatctttactggacagactatgctctggaaacaatt
gaagtctccaaaattgatgggagccacaggactgtgctgattagtaaaaa
cctaacaaatccaagaggactagcattagatcccagaatgaatgagcatc
tactgttctggtctgactggggccaccaccctcgcatcgagcgagccagc
atggacggcagcatgcgcactgtcattgtccaggacaagatcttctggcc
ctgcggcttaactattgactaccccaacagactgctctacttcatggact
cctatcttgattacatggacttttgtgattataatggacaccatcggaga
caggtgatagccagtgatttgattatacggcacccctatgccctaactct
ctttgaagactctgtgtactggactgaccgtgctactcgtcgggttatgc
gagccaacaagtggcatggagggaaccagtcagttgtaatgtataatatt
caatggccccttgggattgttgcggttcatccttcgaaacaaccaaattc
cgtgaatcctgtgccttttccgctgcagccatctctgcctgctttcctca
caggggcctcatttttactcctgtgtttgtccttcaggatggagtctgtc
tcctgatctcctgaattgcttgagagatgatcaacctttcttaataactg
taaggcaacatataatttttggaatctcccttaatcctgaggtgaagagc
aatgatgctatggtccccatagcagggatacagaatggtttagatgttga
atttgatgatgctgagcaatacatctattgggttgaaaatccagggtgaa
attcacagagtgaagacagatggcaccaacaggacagtatttgcttctat
atctatggtggggccttctatgaacctggccttagattggatttcaagaa
acctttattctaccaatcctagaactcagtcaatcgaggttttgacactc
cacggagatatcagatacagaaaaacattgattgccaatgatgggacagc
tcttggagttggctttccaattggcataacgttgatcctgctcgtgggaa
gctgtactggtcagaccaaggaactgacagtggggttcctgccaagatcg
ccagtgtaacatggatggcacatctgtgaaaactctctttactgggaacc
tcgaacacctggagtgtgtcactcttgacatcgaagagcagaaactctac
tgggcagtcactggaagaggagtgattgaaagaggaaacgtggatggaac
agatcgaatgatccttggtacaccagctttcccacccctggggaattgca
gtccatgattctttcctttattatactgatgaacagtatgaggtcattga
aagagttgataaggccactggggccaacaaaatagtcttgagagataatg
ttccaaatctgaggggtcttcaagtttatcacagacgcaatgccgccgaa
tcctcaaatggctgtagcaacaacatgaatgcctgtcagcagatttgcct
gcctgtaccaggaggattgttttcctgcgcctgtgccactggatttaaac
tcaatcctgataatcggtcctgctctccatataactctttcattgttgtt
tcaatgctgtctgcaatcagaggctttagcttggaattgtcagatcattc
agaaaccatggtgccggtggcaggccaaggacgaaacgcactgcatgtgg
atgtggatgtgtcctctggctttatttattggtgtgattttagcagctca
gtggcatctgataatgcgatccgtagaattaaaccagatggatcttctct
gatgaacattgtgacacatggaataggagaaaatggagtccggggtattg
cagtggattgggtagcaggaaatctttatttcaccaatgcctttgtttct
gaaacactgatagaagttctgcggatcaatactacttaccgccgtgttct
tcttaaagtcacagtggacatgcctaggcatattgttgtagatcccaaga
acagatacctcttctgggctgactatgggcagagaccaaagattgagcgt
tctttccttgactgtaccaatcgaacagtgcttgtgtcagagggcattgt
cacaccacggggcttggcagtggaccgaagtgatggctacgtttattggg
ttgatgattctttagatataattgcaaggattcgtatcaatggagagaac
tctgaagtgattcgttatggcagtcgttaccccaactccttatggcatca
ctgtttttgaaaattctatcatatgggtagataggaatttgaaaaagatc
ttccaagccagcaaggaaccagagaacacagagccacccacagtgataag
agacaatatcaactggctaagagatgtgaccattctttgacaagcaagtc
cagccccggtcaccagcagaggtcaacaacaacccttgcttggaaaacaa
tggtgggtgctctcatctctgctttgctctgcctggattgcacaccccaa
aatgtgactgtgcctttgggaccctgcaaagtgatggcaagaattgtgcc
atttcaacagaaaatttcctcatctttgccttgtctaattccttgagaag
cttacacttggaccctgaaaaccatagcccacctttccaaacaataaatg
tggaaagaactgtcatgtctctagactatgacagtgtaagtgatagaatc
tacttcacacaaaatttagcctctggagttggacagatttcctatgccac
cctgtcttcagggatccatactccaactgtcattgcttcaggtataggga
ctgctgatggcattgcctttgactggattactagaagaatttattacagt
gactacctcaaccagatgattaattccatggctgaagatgggtctaaccg
cactgtgatagcccgcgttccaaaaccaagagcaattgtgttagatccct
gccaagggtacctgtactgggctgactgggatacacatgccaaaatcgag
agagccacattgggaggaaacttccgcgtacccattgtgaacagcagtct
ggtcatgcccagtgggctgactctggactatgaagaggaccttctctact
gggtggatgctagtctgcagaggattgaacgcagcactctgacgggcgtg
gatcgtgaagtcattgtcaatgcagccgttcatgcttttggcttgactct
ctatggccagtatatttactggactgacttgtacacacaaagaatttacc
gagctaacaaatatgacgggtcaggtcagattgcaatgaccacaaatttg
ctctcccagcccaggggaatcaacactgttgtgaagaaccagaaacaaca
gtgtaacaatccttgtgaacagtttaatgggggctgcagccatatctgtg
caccaggtccaaatggtgccgagtgccagtgtccacatgagggcaactgg
tatttggccaacaacaggaagcactgcattgtggacaatggtgaacgatg
tggtgcatcttccttcacctgctccaatgggcgctgcatctcggaagagt
ggaagtgtgataatgacaacgactgtggggatggcagtgatgagatggaa
agtgtctgtgcacttcacacctgctcaccgacagccttcacctgtgccaa
tgggcgatgtgtccaatactcttaccgctgtgattactacaatgactgtg
gtgatggcagtgatgaggcagggtgcctgttcagggactgcaatgccacc
acggagtttatgtgcaataacagaaggtgcatacctcgtgagtttatctg
caatggtgtagacaactgccatgataataacacttcagatgagaaaaatt
gccctgatcgcacttgccagtctggatacacaaaatgtcataattcaaat
atttgtattcctcgcgtttatttgtgtgacggagacaatgactgtggaga
taacagtgatgaaaaccctacttattgcaccactcacacgtgcagcagca
gtgagttccaatgcgcatctgggcgctgtattcctcaacattggtattgt
gatcaagaaacagattgttttgatgcctctgatgaacctgcctcttgtgg
tcactctgagcgaacatgcctagctgatgagttcaagtgtgatggtggga
ggtgcatcccaagcgaatggatctgtgacggtgataatgactgtggggat
atgagtgacgaggataaaaggcaccagtgtcagaatcaaaactgctcgga
ttccgagtttctctgtgtaaatgacagacctccggacaggaggtgcattc
cccagtcttgggtctgtgatggcgatgtggattgtactgacggctacgat
gagaatcagaattgcaccaggagaacttgctctgaaaatgaattcacctg
tggttacggactgtgtatcccaaagatattcaggtgtgaccggcacaatg
actgtggtgactatagcgacgagaggggctgcttataccagacttgccaa
cagaatcagtttacctgtcagaacgggcgctgcattagtaaaaccttcgt
ctgtgatgaggataatgactgtggagacggatctgatgagctgatgcacc
tgtgccacaccccagaacccacgtgtccacctcacgagttcaagtgtgac
aatgggcgctgcatcgagatgatgaaactctgcaaccacctagatgactg
tttggacaacagcgatgagaaaggctgtggcattaatgaatgccatgacc
cttcaatcagtggctgcgatcacaactgcacagacaccttaaccagtttc
tattgttcctgtcgtcctggttacaagctcatgtctgacaagcggacttg
tgttgatattgatgaatgcacagagatgccttttgtctgtagccagaagt
gtgagaatgtaataggctcctacatctgtaagtgtgccccaggctacctc
cgagaaccagatggaaagacctgccggcaaaacagtaacatcgaacccta
tctcatttttagcaaccgttactatttgagaaatttaactatagatggct
atttttactccctcatcttggaggactggacaatgttgtggcattagatt
ttgaccgagtagagaagagattgtattggattgatacacagaggcaagtc
attgagagaatgtttctgaataagacaaacaaggagacaatcataaacca
cagactaccagctgcagaaagtctggctgtagactgggtttccagaaagc
tctactggttggatgcccgcctggatggcctctttgtctctgacctcaat
ggtggacaccgccgcatgctggcccagcactgtgtggatgccaacaacac
cttctgctttgataatcccagaggacttgcccttcaccctcaatatgggt
acctctactgggcagactggggtcaccgcgcatacattgggagagtaggc
atggatggaaccaacaagtctgtgataatctccaccaagttagagtggcc
taatggcatcaccattgattacaccaatgatctactctactgggcagatg
cccacctgggttacatagagtactctgatttggagggccaccatcgacac
acggtgtatgatggggcactgcctcaccctttcgctattaccatttttga
agacactatttattggacagattggaatacaaggacagtggaaaagggaa
acaaatatgatggatcaaatagacagacactggtgaacacaacacacaga
ccatttgacatccatgtgtaccatccatataggcagcccattggtgagca
atccctgtggtaccaacaatggtggctgttctcatctctgcctcataagc
caggaggaaaagggttcacttgcgagtgtccagatgacttccgcaccctt
cagctgagtggcagcacctactgcatgcccatgtgctccagcacccagtt
cctgtgcgctaacaatgaaaagtgcattcctatctggtggaaatgtgatg
gacagaaagactgctcagatggctctgatgaactggccctttgcccgcag
cgcttctgccgactgggacagttccagtgcagtgacggcaactgcaccag
cccgcagactttatgcaatgctcaccaaaattgccctgatgggtctgatg
aagaccgtcttctttgtgagaatcaccactgtgactccaatgaatggcag
tgcgccaacaaacgttgcatcccagaatcctggcagtgtgacacatttaa
cgactgtgaggataactcagatgaagacagttcccactgtgccagcagga
cctgccggccgggccagtttcggtgtgctaatggccgctgcatcccgcag
gcctggaagtgtgatgtggataatgattgtggagaccactcggatgagcc
cattgaagaatgcatgagctctgcccatctctgtgacaacttcacagaat
tcagctgcaaaacaaattaccgctgcatcccaaagtgggccgtgtgcaat
ggtgtagatgactgcagggacaacagtgatgagcaaggctgtgaggagag
gacatgccatcctgtgggggatttccgctgtaaaaatcaccactgcatcc
ctcttcgttggcagtgtgatgggcaaaatgactgtggagataactcagat
gaggaaaactgtgctccccgggagtgcacagagagcgagtttcgatgtgt
caatcagcagtgcattccctcgcgatggatctgtgaccattacaacgact
gtggggacaactcagatgaacgggactgtgagatgaggacctgccatcct
gaatattttcagtgtacaagtggacattgtgtacacagtgaactgaaatg
cgatggatccgctgactgtttggatgcgtctgatgaagctgattgtccca
cacgctttcctgatggtgcatactgccaggctactatgttcgaatgcaaa
aaccatgtttgtatcccgccatattggaaatgtgatggcgatgatgactg
tggcgatggttcagatgaagaacttcacctgtgcttggatgttccctgta
attcaccaaaccgtttccggtgtgacaacaatcgctgcatttatagtcat
gaggtgtgcaatggtgtggatgactgtggagatggaactgatgagacaga
ggagcactgtagaaaaccgacccctaaaccttgtacagaatatgaatata
agtgtggcaatgggcattgcattccacatgacaatgtgtgtgatgatgcc
gatgactgtggtgactggtccgatgaactgggttgcaataaaggaaaaga
aagaacatgtgctgaaaatatatgcgagcaaaattgtacccaattaaatg
aaggaggatttatctgctcctgtacagctgggttcgaaaccaatgttttt
gacagaacctcctgtctagatatcaatgaatgtgaacaatttgggacttg
tccccagcactgcagaaataccaaaggaagttatgagtgtgtctgtgctg
atggcttcacgtctatgagtgaccgccctggaaaacgatgtgcagctgag
ggtagctctcctttgttgctactgcctgacaatgtccgaattcgaaaata
taatctctcatctgagaggttctcagagtatcttcaagatgaggaatata
tccaagctgttgattatgattgggatcccaaggacataggcctcagtgtt
gtgtattacactgtgcgaggggagggctctaggtttggtgctatcaaacg
tgcctacatccccaactttgaatccggccgcaataatcttgtgcaggaag
ttgacctgaaactgaaatacgtaatgcagccagatggaatagcagtggac
tgggttggaaggcatatttactggtcagatgtcaagaataaacgcattga
ggtggctaaacttgatggaaggtacagaaagtggctgatttccactgacc
tggaccaaccagctgctattgctgtgaatcccaaactagggcttatgttc
tggactgactggggaaaggaacctaaaatcgagtctgcctggatgaatgg
agaggaccgcaacatcctggttttcgaggaccttggttggccaactggcc
tttctatcgattatttgaacaatgaccgaatctactggagtgacttcaag
gaggacgttattgaaaccataaaatatgatgggactgataggagagtcat
tgcaaaggaagcaatgaacccttacagcctggacatctttgaagaccagt
tatactggatatctaaggaaaagggagaagtatggaaacaaaataaattt
gggcaaggaaagaaagagaaaacgctggtagtgaacccttggctcactca
agttcgaatctttcatcaactcagatacaataagtcagtgcccaaccttt
gcaaacagatctgcagccacctctgccttctgagacctggaggatacagc
tgtgcctgtccccaaggctccagctttatagaggggagcaccactgagtg
tgatgcagccatcgaactgcctatcaacctgccccccccatgcaggtgca
tgcacggaggaaattgctattttgatgagactgacctccccaaatgcaag
tgtcctagcggctacaccggaaaatattgtgaaatggcgttttcaaaagg
catctctccaggaacaaccgcagtagctgtgctgttgacaatcctcttga
tcgtcgtaattggagctctggcaattgcaggattcttccactatagaagg
accggctcccttttgcctgctctgcccaagctgccaagcttaagcagtct
cgtcaagccctctgaaaatgggaatggggtgaccttcagatcaggggcag
atcttaacatggatattggagtgtctggttttggacctgagactgctatt
gacaggtcaatggcaatggtgaagactttgtcatggaaatggggaagcag
cccataatatttgaaaacccaatgtactcagccagagacagtgctgtcaa
agtggttcagccaatccaggtgactgtatctgaaaatgtggataataaga
attatggaagtcccataaacccttctgagatagttccagagacaaaccca
acttcaccagctgctgatggaactcaggtgacaaaatggaatctcttcaa
acgaaaatctaaacaaactaccaactttgaaaatccaatctatgcacaga
tggagaacgagcaaaaggaaagtgttgctgcgacaccacctccatcacct
tcgctccctgctaagcctaagcctccttcgagaagagacccaactccaac
ctattctgcaacagaagacacttttaaagacaccgcaaatcttgttaaag
aagactctgaagtatagctataccagctatttagggaataattagaaaca
cacttttgcacatatattttttacaaacagatgaaaaaagttaacattca
gtactttatgaaaaaaatatatttttccctgtttgcctatagttggaggt
atcctgtgtgtctttttttacttatgccgtctcatatttttacaaataat
tatcacaatgtactatatgtatatctttgcactgaagttgtcgaaggtaa
tactataaatatattgtatatttgtaaattttggaaagattatcctgtta
ctgaatttgctaataaagatgtctgctgatttggttggtgatcattatag
taaatgatccaacaagaaaaggaattgactgggg
DEFINITION Homo sapiens low density lipoprotein
receptor-related protein 3 (LRP3), mRNA.
ACCESSION XM_036573

SEQ ID NO:26

Tacgggcctgcaccgccaccgcacaaagacgcctcgggagccgccgcctg
cacccgggccgcagcagccacgccagccggagcccgagccctagcccgag
cccgagcccgagccgcagccagagccagagccggagccgcagccggaacc
ggagccggagccgcggggcaggaggcggcgcccgcgggcggccgggcccg
gcatggagaagcgcgcggccgcggggctggagggcgcgccgggcgcccgg
gcgcagctggccgtcgtctgtctggtgaacatctttctcaccgggagact
cagcagtgcggttcctgccttagcggcctgcagtgggaagctggagcagc
acacggagcggcgtggggtcatctacagcccggcctggcccctcaactac
ccgccaggcaccaactgcagctggtacatccagggcgaccgtggtgacat
gattaccatcagcttccgcaactttgacgtggaggagtcccaccagtgct
cccggactggctcctgctgggcccagcagccccaccccgccaggaggcct
tccgcctctgtggctccgccatcccacctgccttcatctctgcccgcgac
catgtctggattttcttccactcagacgcctccagctccggccaggccca
gggcttccgtctgtcttacatccgagggaagctgggccaggcatcctgcc
aggcagatcagttccgctgtgacaacggcaagtgcctgcccggcccgtgg
cagtgcaacacggtggacgagtgtggagacggctctgatgagggcaactg
ctcggcgcccgcctccgagcctccaggcagcctgtgccccggggggacct
tcccatgcagcggggcgcgctccacgcgctgcctgcctgtggagcggcgc
tgtgacggcttgcaggactgcggcgacggctcggatgaggcgggctgccc
cgacctggcgtgcggccggcggctgggcagcttctacggctcctttgcct
ccccagacctgttcggcgccgctcgcgggccctcagaccttactgcacgt
ggctggtggacacacaggactcccggcgggtgctgctgcagctggaatgc
ggctgggctatgacgactacgtgcaggtatacgagggcctgggcgagcgc
ggggaccgcctgctgcagacgctgtcctaccgcagcaaccaccggcccgt
gagcctggaggccgcccagggccgcctcactgtggcctaccacgcgcgcg
cccgcagcgccggccacggcttcaatgccacctaccaggtgaagggctat
tgcctcccctgggagcagccgtgcgggagcagtagtgacagtgacggggg
cagcctgggcgaccagggctgcttctcagagccacagcgctgtgatggct
ggtggcattgtgccagcggccgagacgagcagggctgccctgcctgcccg
cccgaccagtacccctgcgagggtggcagtggtctgtgctacacgcctgc
cgaccgctgcaacaaccagaaaagctgtcccgacggcgccgacgagaaga
actgcttctcctgccagcccggcaccttccactgcggtaccaacctgtgc
atcttcgagacgtggcgctgtgacggccaggaagactgccaggacggcag
cgatgagcatgggtgcctggccgccgtgccccgcaaggtcatcacggcgg
cgctcattggcagcctggtgtgtgtggcctgctgctggtcatcgcgctgg
gctgcgccttcaagctctactcactgcgcacgcaggaatacagggccttc
gagacccagatgacgcgcctggaggctgagttcgtgcggcgggaggcacc
cccatcctatggtcagctcatcgcccagggcctcattccacccgtggagg
actttcctgtctacagtgcgtcccaggcctctgtgctgcagaatcttcgc
acagccatgcggagacagatgcgtcggcacgcctcccgccgggggccctc
ccgccgccgcctcggccgcctctggaaccggctctttcaccggccgcggg
cgccccgaggccagatcccactgctgaccgcagcacgcccctcacagacc
gtgctgggcgatggcttcctccagcctgctccaggggctgcccccgaccc
cccagcaccgctcatggacacaggcagcaccagggcggccggagacaggc
cccccagtgcccccggccgtgcaccggaggtgggaccttcagggccaccc
ttgccctcgggcctgcgagacccagagtgcaggcccgtggacaaggacag
aaaggtctgcagggagccactggtagacggcccagctcctgcagatgcac
ctcgggagccctgctcagcccaggacccgcacccccaggtctccactgcc
agcagcaccctgggcccccactcgccagagccactgggggtctgcaggaa
ccccccgcccccctgctccccaatgctggaggccagcgatgatgaggccc
tgttggtctgttgaccgctgggctcgctggtgaccgccacgccccgcttt
gtaaccagggaatacacagtcattt
DEFINITION Homo sapiens low density lipoprotein
receptor-related protein 4 (LRP4), mRNA.
ACCESSION XM_035037

SEQ ID NO:27

Caggcatgaacagtttcctcatcttcgccaggaggatagacattcgcatg
gtctccctggacatcccttattttgctgatgtggtggtaccaatcaacat
taccatgaagaacaccattgccattggagtagacccccaggaaggaaagg
tgtactggtctgacagcacactgcacaggatcagtcgtgccaatctggat
ggctcacagcatgaggacatcatcaccacagggctacagaccacagatgg
gctcgcggttgatgccattggccggaaagtatactggacagacacgggaa
caaaccggattgaagtgggcaacctggacgggtccatgcggaaagtgttg
gtgtggcagaaccttgacagtccccgggccatcgtactgtaccatgagat
ggggtttatgtactggacagactggggggagaatgccaagttagagcggt
ccggaatggatggctcagaccgcgcggtgctcatcaacaacaacctagga
tggcccaatggactgactgtggacaaggccagctcccaactgctatgggc
cgatgcccacaccgagcgaattgaggctgctgacctgaatggtgccaatc
ggcatacattggtgtcaccggtgcagcacccatatggcctcaccctgctc
gactcctatatctactggactgactggcagactcggagcatccaccgtgc
tgacaagggtactggcagcaatgtcatcctcgtgaggtccaacctgccag
gcctcatggacatgcaggctgtccaccgggcacagccactaggttttaac
aagtgcggctcgagaaatggcggctgctcccacctctgcttgcctcggcc
ttctggcttctcctgtgcctgccccactggcatccagctgaagggagatg
ggaagacctgtgatccctctcctgagacctacctgctcttctccagccgt
ggctccatccggcgtatctcactggacaccagtgaccacaccgatgtgca
tgtccctgttcctgagctcaacaatgtcatctccctggactatgacagcg
tggatggaaaggtctattacacagatgtgttcctggatgttatcaggcga
gcagacctgaacggcagcaacatggagacagtgatcgggcgagggctgaa
gaccactgacgggctggcagtggactgggtggccaggaacctgtactgga
cagacacaggtcgaaataccattgaggcgtccaggctggatggtcctgcc
gcaaagtactgatcaacaatagcctggatgagccccgggccattgctgtt
ttccccaggaaggggtacctcttctggacagactggggccacattgccaa
gatcgaacgggcaaacttggatggttctgagcggaaggtcctcatcaaca
cagacctgggttggcccaatggccttaccctggactatgatacccgcagg
atctactgggtggatgcgcatctggaccggatcgagagtgctgacctcaa
tgggaaactgcggcaggtcttggtcagccatgtgtcccacccctttgccc
tcacacagcaagacaggtggtatctactggacagactggcagaccaagtc
aatccagcgtgttgacaaatactcaggccggaacaaggagacagtgctgg
caaatgtggaaggactcatggatatcatcgtggtttcccctcagcggcag
acagggaccaatgcctgtggtgtgaacaatggtggctgcacccacctctg
ctttgccagagcctcggacttcgtatgtgcctgtcctgacgaacctgata
gccggccctgctcccttgtgcctggcctggtaccaccagctcctagggct
actggcatgagtgaaaagagcccagtgctacccaacacaccacctaccac
cttgtattcttcaaccacccggacccgcacgtctctggaggaggtggaag
gaagatgctctgaaagggatgccaggctgggcctctgtgcacgttccaat
gacgctgttcctgctgctccaggggaaggacttcatatcagctacgccat
tggtggactcctcagttattctgctgattttggtggtgattgcagctttg
atgctgtacagacacaaaaaatccaagttcactgatcctggaatggggaa
cctcacctacagcaacccctcctaccgaacatccacacggaagtgaagat
tgaagcaatccccaaaccagccatgtacaaccagctgtgctataagaaag
agggagggcctgaccataactacaccaaggagaagatcaagatcgtagag
ggaatctgcctcctgtctggggatgatgctgagtgggatgacctcaagca
actgcgaagctcacgggggggcctcctccgggatcatgtatgcatgaaga
cagacacggtgtccatccaggccagctctggctccctggatgacacagag
acggagcagctgttacaggaagagcagtctgagtgtagcagcgtccatac
tgcagccactccagaaagacgaggctctctgccagacacgggctggaaac
atgaacgcaagctctcctcagagagccaggtctaaatgcccacattctct
tccctgcctgcctgttccttctcctttatggacgtctagtccttgtgctc
gcttacaccgcaggccccgcttctgtgtgcttgtcctcctcctcctccca
ccccataactgttcctaagccttcaccggagctgtttaccacgtgagtcc
ataactacctgtgcacaagaaatgatggcacatcacgagaatttagacct
ggattttaccatgaacctcacatcttgtactccatcctgggccccctgaa
actgcttattcgtgattcctcaccagcgtagagctccacctcccctttcc
ccagtaccctcagtgcctgcttctcagtgctgatgcagctgatgacccag
gactgcgctctgccccatcacagccagcatgactgcttctctgagagaac
ttgcccatcaggggctgggacatgggggtgtgggtaaagacagggatgaa
ggatagaggctgagagaagaaggaagaatcagcccagcaggtatgggcat
ctgggaaacctccagcctcaagtgtttagtaacatgaaaaagctttgggg
ggtagttggatctgggtgtctggtccattgctggcagtggacattattct
tgccctaagagacactgccttttcagcagcagatactggtgagatggggg
tggctcaggctgttcttcctcctcctagaatgtctggagctgtttctaca
ttcagataactgggtcccctatcacaaggctactggctaataggaattcc
ctcctggtgccaccactggccagtacctttcctaagtctttgctcaaatt
aaccaggttgtgagccagtggcttgagtgaatgttaggccttgggggctg
agtctctgaaaagtctaagaagctctgcctagaccaaatatggtatacct
cctgacccctctctccctcatgtcctgggattctggggaagagacctaga
aacaagctttcaaagaaaaaccagaagttgtcataaatggtcagaaagaa
cgatcaggttggagacttgggaaacccagggcctaaagagaagtatccat
gagggtcaaacttcctgttgaacttcctatgttctttctcaagtgctcag
ggatctaagttagtggacagcaagcctgtggctacggggtggtgatgttc
ctcttccagctgtcccctcagctaaggggcttagtttccatgtgggatgc
catcacttggttcatgctcattcacacaaagggcacgtgtctcagccggt
atcagggaaattgagacttatttttgccctaaaacgtctccctagctgtt
cttcgtggggtttttttgtttgtttttttgcctaatttgctttttctgac
caagccttgtggcaccagcaatctccaaagtcctgtggtgggagggctga
ataaataaaaatacaaagaggtgggtaaggagtaggaaggtagagagcac
cactgatgaggccctcctagcccatggcagacccagacctcttctccccc
aggaattagaagtggcaggagagaacaacaggggctgggaatggagggga
gaatttctaggggaagtttcctgagttgaaacttctcctgtggttactgg
tattgagaaatcagctaccaaagtgaaaaaggacaagatcaattcttttc
tagtcagttctaagactgctagagagagataccaggcccttagccttgct
ctcagtagcgtcagccccagttctgagcctccccacattacacttaacaa
gcagtaaaggagtgagcactttgggtccttagactcacgtctggggagga
agagcaagtagaaaagtggcattttcttgattggaaagggggaaggatct
tattgcacttgggctgttcagaatgtagaaaggacatatttgaggaagta
tctatttgagcactgatttactctgtaaaaagcaaaatctctctgtccta
aactaatggaagcgattctcccatgctcatgtgtaatggttttaacgtta
ctcactggagagattggactttctggagttatttaaccactatgttcagt
attttaggactttatgataatttaatataaatttagcttttccttaatc
DEFINITION Homo sapiens low density lipoprotein
receptor-related protein 5 (LRP5), mRNA.
ACCESSION NM_002335

SEQ ID NO:28

Atggagcccgagtgagcgcggcgcgggcccgtccggccgccggacaacat
ggaggcagcgccgcccgggccgccgtggccgctgctgctgctgctgctgc
tgctgctggcgctgtgcggctgcccggcccccgccgcggcctcgccgctg
gtgctatttgccaaccgccgggacgtacggctggtggacgccggcggagt
caagctggagtccaccatcgtggtcagcggcctggaggatgcggccgcag
tggacttccagttttccaagggagccgtgtactggacagacgtgagcgag
gaggccatcaagcagacctacctgaaccagacgggggccgccgtgcagaa
cgtggtcatctccggcctggtctctcccgacggcctcgcctgcgactggg
tgggcaagaagctgtactggacggactcagagaccaaccgcatcgaggtg
gccaacctcaatggcacatcccggaaggtgctcttctggcaggaccttga
ccagccgagggccatcgccttggaccccgctcacgggtacatgtactgga
cagactggggtgagacgccccggattgagcgggcagggatggatggcagc
acccggaagatcattgtggactcggacatttactggcccaatggactgac
catcgacctggaggagcagaagctctactgggctgacgccaagctcagct
tcatccaccgtgccaacctggacggctcgttccggcagaaggtggtggag
ggcagcctgacgcaccccttcgccctgacgctctccggggacactctgta
ctggacagactggcagacccgctccatccatgcctgcaacaagcgcactg
gggggaagaggaaggagatcctgagtgccctctactcacccatggacatc
caggtgctgagccaggagcggcagcctttcttccacactcgctgtgagga
ggacaatggcggctgctcccacctgtgcctgctgtccccaagcgagcctt
tctacacatgcgcctgccccacgggtgtgcagctgcaggacaacggcagg
acgtgtaaggcaggagccgaggaggtgctgctgctggcccggcggacgga
cctacggaggatctcgctggacacgccggactttaccgacatcgtgctgc
aggtggacgacatccggcacgccattgccatcgactacgacccgctagag
ggctatgtctactggacagatgacgaggtgcgggccatccgcagggcgta
cctggacgggtctggggcgcagacgctggtcaacaccgagatcaacgacc
ccgatggcatcgcggtcgactgggtggcccgaaacctctactggaccgac
acgggcacggaccgcatcgaggtgacgcgcctcaacggcacctcccgcaa
gatcctggtgtcggaggacctggacgagccccgagccatcgcactgcacc
ccgtgatgggcctcatgtactggacagactggggagagaaccctaaaatc
gagtgtgccaacttggatgggcaggagcggcgtgtgctggtcaatgcctc
cctcgggtggcccaacggcctggccctggacctgcaggaggggaagctct
actggggagacgccaagacagacaagatcgaggtgatcaatgttgatggg
acgaagaggcggaccctcctggaggacaagctcccgcacattttcgggtt
cacgctgctgggggacttcatctactggactgactggcagcgccgcagca
tcgagcgggtgcacaaggtcaaggccagccgggacgtcatcattgaccag
ctgcccgacctgatggggctcaaagctgtgaatgtggccaaggtcgtcgg
aaccaacccgtgtgcggacaggaacggggggtgcagccacctgtgcttct
tcacaccccacgcaacccggtgtggctgccccatcggcctggagctgctg
agtgacatgaagacctgcatcgtgcctgaggccttcttggtcttcaccag
cagagccgccatccacaggatctccctcgagaccaataacaacgacgtgg
ccatcccgctcacgggcgtcaaggaggcctcagccctggactttgatgtg
tccaacaaccacatctactggacagacgtcagcctgaagaccatcagccg
cgccttcatgaacgggagctcggtggagcacgtggtggagtttggccttg
actaccccgagggcatggccgttgactggatgggcaagaacctctactgg
gccgacactgggaccaacagaatcgaagtggcgcggctggacgggcagtt
ccggcaagtcctcgtgtggagggacttggacaacccgaggtcgctggccc
tggatcccaccaagggctacatctactggaccgagtggggcggcaagccg
aggatcgtgcgggccttcatggacgggaccaactgcatgacgctggtgga
caaggtgggccgggccaacgacctcaccattgactacgctgaccagcgcc
tctactggaccgacctggacaccaacatgatcgagtcgtccaacatgctg
ggtcaggagcgggtcgtgattgccgacgatctcccgcacccgttcggtct
gacgcagtacagcgattatatctactggacagactggaatctgcacagca
ttgagcgggccgacaagactagcggccggaaccgcaccctcatccagggc
cacctggacttcgtgatggacatcctggtgttccactcctcccgccagga
tggcctcaatgactgtatgcacaacaacgggcagtgtgggcagctgtgcc
ttgccatccccggcggccaccgctgcggctgcgcctcacactacaccctg
gaccccagcagccgcaactgcagcccgcccaccaccttcttgctgttcag
ccagaaatctgccatcagtcggatgatcccggacgaccagcacagcccgg
atctcatcctgcccctgcatggactgaggaacgtcaaagcatcgactatg
acccactggacaagttcatctactgggtggatgggcgccagaacatcaag
cgagccaaggacgacgggacccagccctttgttttgacctctctgagcca
aggccaaaacccagacaggcagccccacgacctcagcatcgacatctaca
gccggacactgttctggacgtgcgaggccaccaataccatcaacgtccac
aggctgagcggggaagccatgggggtggtgctgcgtggggaccgcgacaa
gcccagggccatcgtcgtcaacgcggagcgagggtacctgtacttcacca
acatgcaggaccgggcagccaagatcgaacgcgcagccctggacggcacc
gagcgcgaggtcctcttcaccaccggcctcatccgccctgtggccctggt
ggtagacaacacactgggcaagctgttctgggtggacgcggacctgaagc
gcattgagagctgtgacctgtcaggggccaaccgcctgaccctggaggac
gccaacatcgtgcagcctctgggcctgaccatccttggcaagcatctcta
ctggatcgaccgccagcagcagatgatcgagcgtgtggagaagaccaccg
gggacaagcggactcgcatccagggccgtgtcgcccacctcactggcatc
catgcagtggaggaagtcagcctggaggagttctcagcccacccatgtgc
ccgtgacaatggtggctgctcccacatctgtattgccaagggtgatggga
caccacggtgctcatgcccagtccacctcgtgctcctgcagaacctgctg
acctgtggagagccgcccacctgctccccggaccagtttgcatgtgccac
aggggagatcgactgtatccccggggcctggcgctgtgacggctttcccg
agtgcgatgaccagagcgacgaggagggctgccccgtgtgctccgccgcc
cagttcccctgcgcgcggggtcagtgtgtggacctgcgcctgcgctgcga
cggcgaggcagactgtcaggaccgctcagacgaggcggactgtgacgcca
tctgcctgcccaaccagttccggtgtgcgagcggccagtgtgtcctcatc
aaacagcagtgcgactccttccccgactgtatcgacggctccgacgagct
catgtgtgaaatcaccaagccgccctcagagacagcccggcccacagcag
tgccatcgggcccgtcattggcatcatcctctctctcttcgtcatgggtg
gtgtctattttgtgtgccagcgcgtggtgtgccagcgctatgcgggggcc
aacgggcccttcccgcacgagtatgtcagcgggaccccgcacgtgcccct
caatttcatagccccgggcggttcccagcatggccccttcacaggcatcg
catgcggaaagtccatgatgagctccgtgagcctgatggggggccggggc
ggggtgcccctctacgaccggaaccacgtcacaggggcctcgtccagcag
ctcgtccagcacgaaggccacgctgtacccgccgatcctgaacccgccgc
ccctccccggccacggacccctccctgtacaacatggacatgttctactc
ttcaaacattccggccactgtgagaccgtacaggccctacatcattcgag
gaatggcgcccccgacgacgccctgcagcaccgacgtgtgtgacagcgac
tacagcgccagccgctggaaggccagcaagtactacctggatttgaactc
ggactcagacccctatccacccccacccacgccccacagccagtacctgt
cggcggaggacagctgcccgccctcgcccgccaccgagaggagctacttc
catctcttcccgccccctccgtccccctgcacggactcatcctgacctcg
gccgggccactctggcttctctgtgcccctgtaaatagttttaaatatga
acaaagaaaaaaatatattttatgatttaaaaaataaatataattgggat
tttaaaaacatgagaaatgtgaactgtgatggggtgggcagggctgggag
aactttgtacagtggaacaaatatttataaacttaattttgtaaaacag
DEFINITION Homo sapiens low density lipoprotein
receptor-related protein 6 (LRP6), Mrna.
ACCESSION NM_002336

SEQ ID NO:29

Gcggccgccccggctcctcgcctcccccacttctggccacccctcgccgg
tgagagaagagaacgcgagaagggaagatgggggccgtcctgaggagcct
cctggcctgcagcttctgtgtgctcctgagagcggcccctttgttgcttt
atgcaaacagacgggacttgcgattggttgatgctacaaatggcaaagag
aatgctacgattgtagttggaggcttggaggatgcagctgcggtggactt
tgtgtttagtcatggcttgatatactggagtgatgtcagcgaagaagcca
ttaaacgaacagaatttaacaaaactgagagtgtgcagaatgttgttgtt
tctggattattgtcccccgatgggctggcatgtgattggcttggagaaaa
attgtactggacagattctgaaactaatcggattgaagtttctaatttag
atggatctttacgaaaagttttattttggcaagagttggatcaacccaga
gctattgccttagatccttcaagtgggttcatgtactggacagactgggg
agaagtgccaaagatagaacgtgctggaatggatggttcaagtcgcttca
ttataataaacagtgaaatttactggccaaatggactgactttggattat
gaagaacaaaagctttattgggcagatgcaaaacttaatttcatccacaa
atcaaatctggatggaacaaatcggcaggcagtggttaaaggttcccttc
cacatccttttgccttgacgttatttgaggacatattgtactggactgac
tggagcacacactccattttggcttgcaacaagtatactggtgagggtct
gcgtgaaatccattctgacatcttctctcccatggatatacatgccttca
gccaacagaggcagccaaatgccacaaatccatgtggaattgacaatggg
ggttgttcccatttgtgtttgatgtctccagtcaagcctttttatcagtg
tgcttgccccactggggtcaaactcctggagaatggaaaaacctgcaaag
atggtgccacagaattattgcttttagctcgaaggacagacttgagacgc
atttctttggatacaccagattttacagacattgttctgcagttagaaga
catccgtcatgccattgccatagattacgatcctgtggaaggctacatct
actggactgatgatgaagtgagggccatacgccgttcatttatagatgga
tctggcagtcagtttgtggtcactgctcaaattgcccatcctgatggtat
tgctgtggactgggttgcacgaaatctttattggacagacactggcactg
atcgaatagaagtgacaaggctcaatgggaccatgaggaagatcttgatt
tcagaggacttagaggaaccccgggctattgtgttagatcccatggttgg
gtacatgtattggactgactggggagaaattccgaaaattgagcgagcag
ctctggatggttctgaccgtgtagtattggttaacacttctcttggttgg
ccaaatggtttagccttggattatgatgaaggcaaaatatactggggaga
tgccaaaacagacaagattgaggttatgaatactgatggcactgggagac
gagtactagtggaagacaaaattcctcacatatttggatttactttgttg
ggtgactatgtttactggactgactggcagaggcgtagcattgaaagagt
tcataaacgaagtgcagagagggaagtgatcatagatcagctgcctgacc
tcatgggcctaaaggctacaaatgttcatcgagtgattggttccaacccc
tgtgctgaggaaaacgggggatgtagccatctctgcctctatagacctca
gggccttcgctgtgcttgccctattggctttgaactcatcagtgacatga
agacctgcattgtcccagaggctttccttttgttttcacggagagcagat
atcagacgaatttctctggaaacaaacaataataatgtggctattccact
cactggtgtcaaagaagcttctgctttggattttgatgtgacagacaacc
gaatttattggactgatatatcactcaagaccatcagcagagcctttatg
aatggcagtgcactggaacatgtggtagaattcggcttagattatccaga
aggcatggcagtagactggcttgggaagaacttgtactgggcagacacag
gaacgaatcgaattgaggtgtcaaagttggatgggcagcaccgacaagtt
ttggtgtggaaagacctagatagtcccagagctctcgcgttggaccctgc
cgaaggatttatgtattggactgaatggggtggaaaacctaagatagaca
gagctgcaatggatggaagtgaacgtactaccttagttccaaatgtgggg
cgggcaaacggcctaactattgattatgctaaaaggaggctttattggac
agacctggacaccaacttaatagaatcttcaaatatgcttgggctcaacc
gtgaagttatagcagatgacttgcctcatccttttggcttaactcagtac
caagattatatctactggacggactggagccgacgcagcattgagcgtgc
caacaaaaccagtggccaaaaccgcaccatcattcagggccatttggatt
atgtgatggacatcctcgtctttcactcatctcgacagtcagggtggaat
gaatgtgcttccagcaatgggcactgctcccacctctgcttggctgtgcc
agttgggggttttgtttgtggatgccctgcccactactctcttaatgctg
acaacaggacttgtagtgctcctacgactttcctgctcttcagtcaaaag
agtgccatcaaccgcatggtgattgatgaacaacagagccccgacatcat
ccttcccatccacagccttcggaatgtccgggccattgactatgacccac
tggacaagcaactctattggattgactcacgacaaaacatgatccgaaag
gcacaagaagatggcagccagggctttactgtggttgtgagctcagttcc
gagtcagaacctggaaatacaaccctatgacctcagcattgatatttaca
gccgctacatctactggacttgtgaggctaccaatgtcattaatgtgaca
agattagatgggagatcagttggagtggtgctgaaaggcgagcaggacag
acctcgagccattgtggtaaacccagagaaagggtatatgtatttttacc
aatcttcaggaaaggtctcctaaaattgaacgggctgctttggatgggac
agaacgggaggtcctctttttcagtggcttaagtaaaccaattgctttag
cccttgatagcaggctgggcaagctcttttgggctgattcagatctccgg
cgaattgaaagcagtgatctctcaggtgctaaccggatagtattagaaga
ctccaatatcttgcagcctgtgggacttactgtgtttgaaaactggctct
attggattgataaacagcagcaaatgattgaaaaaattgacatgacaggt
cgagagggtagaaccaaagtccaagctcgaattgcccagcttagtgacat
tcatgcagtaaaggagctgaaccttcaagaatacagacagcacccttgtg
ctcaggataatggtggctgttcacatatttgtcttgtaaagggggatggt
actacaaggtgttcttgccccatgcacctggttctacttcaagatgagct
atcatgtggagaacctccaacatgttctcctcagcagtttacttgtttca
cgggggaaattgactgtatccctgtggcttggcggtgcgatgggtttact
gaatgtgaagaccacagtgatgaactcaattgtcctgtatgctcagagtc
ccagttccagtgtgccagtgggcagtgtattgatggtgccctccgatgca
atggagatgcaaactgccaggacaaatcagatgagaagaactgtgaagtg
ctttgtttaattgatcagttccgctgtgccaatggtcagtgcattggaaa
gcacaagaagtgtgatcataatgtggattgcagtgacaagtcagatgaac
tggattgttatccgactgaagaaccagcaccacaggccaccaatacagtt
ggttctgttattggcgtaattgtcaccatttttgtgtctggaactgtata
ctttatctgccagaggatgttgtgtccacgtatgaagggagatggggaaa
ctatgactaatgactatgtagttcatggaccagcttctgtgcctcttggt
tatgtgccacacccaagttctttgtcaggatctcttccaggaatgtctcg
aggtaaatcaatgatcagctccctcagtatcatggggggaagcagtggac
ccccctatgaccgagcccatgttacaggagcatcatcaagtagttcttca
agcaccaaaggcacttacttccctgcaattttgaaccctccaccatcccc
agccacagagcgatcacattacactatggaatttggatattcttcaaaca
gtccttccactcataggtcatacagctacaggccatatagctaccggcac
tttgcaccccccaccacaccctgcagcacagatgtttgtgacagtgacta
tgctcctagtcggagaatgacctcagtggcaacagccaagggctatacca
gtgacttgaactatgattcagaacctgtgcccccacctcccacaccccga
agccaatacttgtcagcagaggagaactatgaaagctgcccaccttctcc
atacacagagaggagctattctcatcacctctacccaccgccaccctctc
cctgtacagactcctcctgaggaggggccctcctcctctgactgcctcca
acgtaaaaatgtaaatataaatttggttgagatctggagggggggaggga
gctattagagaaggatgaggcagaccatgtacagttaaaattataaaatg
gggtagggaatactggagatatttgtacagaagaaaaggatatttatata
ttttcttaaaacagcagatttgctgcttgtgccataaaagtttgtataaa
aaaaatttgtactaaaagttttatttttgcaaactaaatacacaaagcat
gccttaaacccagtgaagcaactgagtacaaaggaaacaggaataataaa
ggcatcactgaccaggaatatctgggctttattgataccaaaaaaaaaaa
aa
DEFINITION Homo sapiens low density lipoprotein
receptor-related protein 8, apolipoprotein e
receptor (LRP8), mRNA.
ACCESSION XM_016537

SEQ ID NO:30

Tgctgctgctgctgctgctgctgctgctgcagctccagcatcttgcggcg
gcagcggctgatccgctgctggcggccaagggccggccaaggattgcgaa
aaggaccaattccagtgccggaacgagcgctgcatcccctctgtgtggag
atgcgacgaggacgatgactgcttagaccacagcgacgaggacgactgcc
ccaagaagacctgtgcagacagtgacttcacctgtgacaacggccactgc
atccacgaacggtggaagtgtgacggcgaggaggagtgtcctgatggctc
cgatgagtccgaggccacttgcaccaagcaggtgtgtcctgcagagaagc
tgagctgtggacccaccagccacaagtgtgtacctgcctcgtggcgctgc
gacggggagaaggactgcgagggtggagccggatgaggccggctgtgcta
cctcactgggcacctgccgtggggacgagttccagtgtggggatgggaca
tgtgtccttgcaatcaagcactgcaaccaggagcaggactgtccagatgg
gagtgatgaagctggctgcctacaggggctgaacgagtgtctgcacaaca
atggcggctgctcacacatctgcactgacctcaagattggctttgaatgc
acgtgcccagcaggcttccagctcctggaccagaagacctgtggcgacat
tgatgagtgcaaggacccagatgcctgcagccagatctgtgtcaattaca
agggctattttaagtgtgagtgctaccctggctacgagatggacctactg
accaagaactgcaaggctgctggtggcaagagcccatccctaatcttcac
caaccggcacgaggtgcggaggatcgacctggtgaagcggaactattcac
gcctcatccccatgctcaagaatgtcgtggcactagatgtggaagttgcc
accaatcgcatctactggtgtgacctctcctaccgtaagatctatagcgc
ctacatggacaaggccagtgacccgaaagagcaggaggtcctcattgacg
agcagttgcactctccagagggcctggcagtggactgggtccacaagcac
atctactggactgactcgggcaataagaccatctcagtggccacagttga
tggtggccgccgacgcactctcttcagccgtaacctcagtgaaccccggg
ccatcgctgttgaccccctgcgagggttcatgtattggtctgactggggg
gaccaggccaagattgagaaatctgggctcaacggtgtggaccggcaaac
actggtgtcagacaatattgaatggcccaacggaatcaccctggatctgc
tgagccagcgcttgtactgggtagactccaagctacaccaactgtccagc
attgacttcagtggaggcaacagaaagacgctgatctcctccactgactt
cctgagccacccttttgggatagctgtgtttgaggacaaggtgttctgga
cagacctggagaacgaggccattttcagtgcaaatcggctcaatggcctg
gaaatctccatcctggctgagaacctcaacaacccacatgacattgtcat
cttccatgagctgaagcagccaagagctccagatgcctgtgagctgagtg
tccagcctaatggaggctgtgaatacctgtgccttcctgctcctcagatc
tccagccactctcccaagtacacatgtgcctgtcctgacacaatgtggct
gggtccagacatgaagaggtgctaccgagcacctcaatctacctcaacta
cgacgttagcttctaccatgacgaggacagtacctgccaccacaagagcc
cccgggaccaccgtccacagatccacctaccagaaccacagcacagagac
accaagcctgacagctgcagtcccaagctcagttagtgtccccagggctc
ccagcatcagcccgtctaccctaagccctgcaaccagcaaccactcccag
cactatgcaaatgaagacagtaagatgggctcaacagtcactgccgctgt
tatcgggatcatcgtgcccatagtggtgatagccctcctgtgcatgagtg
gatacctgatctggagaaactggaagcggaagaacaccaaaagcatgaat
tttgacaacccagtctacaggaaaacaacagaagaagaagacgaagatga
gctccatatagggagaactgctcagattggccatgtctatcctgcagcaa
tcagcagctttgatcgcccactgtgggcagagccctgtcttggggagacc
agagaaccggaagacccagcccctgccctcaaggagctttttgtcttgcc
gggggaaccaaggtcacagctgcaccaactcccgaagaaccctctttccg
agctgcctgtcgtcaaatccaagcgagtggcattaagccttgaagatgat
ggactaccctgaggatgggatcacccccttcgtgcctcatggaattcagt
cccatgcactacactctggatggtgtatgactggatgaatgggtttctat
atatgggtctgtgtgagtgtatgtgtgtgtgtgattttttttttaaattt
atgttgcggaaaggtaaccacaaagttatgatgaactgcaaacatccaaa
ggatgtgagagtttttctatgtataatgttttatacactttttaactggt
tgcactacccatgaggaattcgtggaatggctactgctgactaacatgat
gcacataaccaaatgggggccaatggcacagtaccttactcatcatttaa
aaactatatttacagaagatgtttggttgctgggggggcttttttaggtt
ttggggcatttgttttttgtaaataagatgattatgctttgtggctatcc
atcaacataagtaaaaaaaaaaaaaaaacacttcaactccctcccccatt
tagattatttattaacatattttaaaaatcagatgagttctataaataat
ttagagaagtgagagtatttatttttggcatgtttggcccaccacacaga
ctctgtgtgtgtatgtgtgtgtttatatgtgtatgtgtgtgacagaaaaa
tctgtagagaagaggcacatctatggctactgttcaaatacataaagata
aatttattttcacacagtccacaaggggtatatcttgtagttttcagaaa
agcctttggaaatctggatcagaaaatagataccatggtttgtgcaatta
tgtagtaaaaaaggcaaatcttttcacctctggctattcctgagacccca
ggaagtcaggaaaagcctttcagctcacccatggctgctgtgactcctac
cagggctttcttggctttggcgaaggtcagtgtacagacattccatggta
ccagagtgctcagaaactcaagataggatatgcctcaccctcagctactc
cttgttttaaagttcagctctttgagtaacttcttcaatttctttcagga
cacttgggttgaattcagtaagtttcctctgaagcaccctgaagggtgcc
atccttacagagctaagtggagacgtttccagatcagcccaagtttacta
tagagactggcccaggcactgaatgtctaggacatgctgtggatgaagat
aaagatggtggaataggttttatcacatctcttatttctcttttcccctt
actctctaccatttcctttatgtggggaaacattttaaggtaataaatag
gttacttaccatcatatgttcatatagatgaaactaatttttggcttaag
tcagaacaactggccaaaattgaagtcatatttgaggggggaaatggcat
acgcaatattatattatattggatatttatgttcacacaggaatttggtt
tactgctttgtaaataaaaggaaaaactccgggtatatgt
DEFINITION Homo sapiens porcupine (MG61), mRNA.
ACCESSION NM_022825

SEQ ID NO:31

Gcaatggccacctttagccgccaggaattttccagcagctactgcaaggc
tgtctcctgcctactgcccagcagggccttgaccagatctggctgctcct
tgccatctgcctcgcctgccgcctcctctggaggctcgggttgccatcct
acctgaagcatgcaagcaccgtggcaggcgggttcttcagcctctaccac
ttcttccagctgcacatggtttgggtcgtgctgctcagcctcctgtgcta
cctcgtgctgttcctctgccgacattcctcccatcgaggcgtcttcctat
ccgtcaccatcctcatctacctactcatgggtgagatgcacatggtagac
accgtgacatggcacaagatgcgaggggcacagatgattgtggccatgaa
ggcagtgtctctgggcttcgacctggaccggggcgaggtgggtacggtgc
cctcgccagtggagttcatgggctacctctacttcgtgggcaccatcgtc
ttcgggccctggatatccttccacagctacctacaaactgtccaaggccg
cccactgagctgccggtggctgcagaaggtggcccggagcctggcactgg
ccctgctgtgccttgtgctgtccacttgcgtgggcccctacctcttcccg
tacttcatccccctcaacggtgaccgcctccttcgcaacaagaaacgcaa
agccaggggcaccatggtaaggtggctgcgagcctacgagagtgctgtct
ccttccacttcagcaactattttgtgggctttctttccgaggccacggcc
acgttggcgggggctggctttaccgaggagaaggatcacctggaatggga
cctgacggtgtccaagccactgaatgtggagctgcctcggtcaatggtgg
aagttgtcacaagctggaacctgcccatgtcttattggctaaataactat
gttttcaagaatgctctccgcctggggaccttctcggctgtgctggtcac
ctatgcagccagcgccctcctacatggcttcagtttccacctggctgcgg
tcctgctgtccctggcttttatcacttacgtggagcatgtcctccggaag
cgcctggctcggatcctcagtgcctgtgtcttgtcaaagcggtgcccgcc
agactgttcgcaccagcatcgcttgggcctgggggtgcgagccttaaact
tgctctttggagctctggccatcttccacctggcctacctgggctccctg
tttgatgtcgatgtggatgacaccacagaggagcagggctacggcatggc
atacactgtccacaagtggtcagagctcagctgggccagtcactgggtca
cttttggatgctggatcttctaccgtctcataggctgaggcacatctgtg
gaccctcataaccctcttaagacccctctcagggtgccactgatggggga
tgagggaaggccctctctctactccttgaccccctccatccttgaccccc
aacacctcaacacacacacacacacacacacacaaaatcacaccattttc
atgcctgtcaatccccaccccaccacaaggcaggaagggggtggtgcctg
ctggggcctagagggggatgcttggggaaacagagaaggggagatccagg
gcctccccgccttccttcttcctttttatatacaatttgttattgtcaaa
taaaagtaggaaatattcaataggct
Extracellular Wnt antagonists:
DEFINITION Homo sapiens dickkopf (Xenopus laevis)
homolog 1 (DKK1), mRNA.
ACCESSION XM_048687

SEQ ID NO:32

Cggcacggtttcgtggggacccaggcttgcaaagtgacggtcattttctc
tttctttctccctcttgagtccttctgagatgatggctctgggcgcagcg
ggagctacccgggtctttgtcgcgatggtagcggcggctctcggcggcca
ccctctgctgggagtgagcgccaccttgaactcggttctcaattccaacg
ctatcaagaacctgcccccaccgctgggcggcgctgcggggcacccaggc
tctgcagtcagcgccgcgccgggaatcctgtacccgggcgggaataagta
ccagaccattgacaactaccagccgtacccgtgcgcagaggacgaggagt
gcggcactgatgagtactgcgctagtcccacccgcggaggggacgcaggc
gtgcaaatctgtctcgcctgcaggaagcgccgaaaacgctgcatgcgtca
cgctatgtgctgccccgggaattactgcaaaaatggaatatgtgtgtctt
ctgatcaaaatcatttccgaggagaaattgaggaaaccatcactgaaagc
tttggtaatgatcatagcaccttggatgggtattccagaagaaccacctt
gtcttcaaaaatgtatcacaccaaaggacaagaaggttctgtttgtctcc
ggtcatcagactgtgcctcaggattgtgttgtgctagacacttctggtcc
aagatctgtaaacctgtcctgaaagaaggtcaagtgtgtaccaagcatag
gagaaaaggctctcatggactagaaatattccagcgttgttactgtggag
aaggtctgtcttgccggatacagaaagatcaccatcaagccagtaattct
tctaggcttcacacttgtcagagacactaaaccagctatccaaatgcagt
gaactccttttatataatagatgctatgaaaaccttttatgaccttcatc
aactcaatcctaaggatatacaagttctgtggtttcagttaagcattcca
ataacaccttccaaaaacctggagtgtaagagctttgtttctttatggaa
ctcccctgtgattgcagtaaattactgtattgtaaattctcagtgtggca
cttacctgtaaatgcaatgaaacttttaattatttttctaaaggtgctgc
actgcctatttttcctcttgttatgtaaattttgtacacattgattgtta
tcttgactgacaaatattctattgaactgaagtaaatcatttcagcttat
agttcttaaaagcataaccctttaccccatttaattctagagtctagaac
gcaaggatctcttggaatgacaaatgataggtacctaaaatgtaacatga
aaatactagcttatttttctgaaatgtactatcttaatgcttaaattata
tttccctttaggctgtgatagtttttgaaataaaatttaacatttaatat
catgaaatgttataagtagacat
DEFINITION Homo sapiens dickkopf (Xenopus laevis)
homolog 2 (DKK2), mRNA.
ACCESSION XM_052645

SEQ ID NO:33

Gcgggtctcgcttgggttccgctaatttctgtcctgaggcgtgagactga
gttcatagggtcctgggtccccgaaccaggaagggttgagggaacacaat
ctgcaagcccccgcgacccaagtgaggggccccgtgttggggtcctccct
ccctttgcattcccaccccctccgggctttgcgtcttcctggggaccccc
tcgccgggagatggccgcgttgatgcggagcaaggattcgtcctgctgcc
tgctcctactggccgcggtgctgatggtggagagctcacagatcggcagt
tcgcgggccaaactcaactccatcaagtcctctctgggcggggagacgcc
tggtcaggccgccaatcgatctgcgggcatgtaccaaggactggcattcg
gcggcagtaagaagggcaaaaacctggggcaggcctacccttgtagcagt
gataaggagtgtgaagttgggaggtattgccacagtccccaccaaggatc
atcggcctgcatggtgtgtcggagaaaaaagaagcgctgccaccgagatg
gcatgtgctgccccagtacccgctgcaataatggcatctgtatcccagtt
actgaaagcatcttaacccctcacatcccggctctggatggtactcggca
cagagatcgaaaccacggtcattactcaaaccatgacttgggatggcaga
atctaggaagaccacacactaagatgtcacatataaaagggcatgaagga
gacccctgcctacgatcatcagactgcattgaagggttttgctgtgctcg
tcatttctggaccaaaatctgcaaaccagtgctccatcagggggaagtct
gtaccaaacaacgcaagaagggttctcatgggctggaaattttccagcgt
tgcgactgtgcgaagggcctgtcttgcaaagtatggaaagatgccaccta
ctcctccaaagccagactccatgtgtgtcagaaaatttgatcaccattga
ggaacatcatcaattgcagactgtgaagttgtgtatttaatgcattatag
catggtggaaaataaggttcagatgcagaagaatggctaaaataagaaac
gtgataagaatatagatgatcac
DEFINITION Homo sapiens dickkopf (Xenopus laevis)
homolog 3 (DKK3), mRNA.
ACCESSION XM_006030

SEQ ID NO:34

Ctatcacaatgagaccaacacagacacgaaggttggaaataataccatcc
atgtgcaccgagaaattcacaagataaccaacaaccagactggacaaatg
gtcttttcagagacagttatcacatctgtgggagacgaagaaggcagaag
gagccacgagtgcatcatcgacgaggactgtgggcccagcatgtactgcc
agtttgccagcttccagtacacctgccagcctgccggggccagaggatgc
tctgcacccgggacagtgagtgctgtggagaccagctgtgtgctggggtc
actgcaccaaaatggccaccaggggcagcaatgggaccatctgtgacaac
cagagggactgccagccggggctgtgctgtgccttccagagaggcctgct
gttccctgtgtgcacacccctgcccgtggagggcgagctttgccatgacc
ccgccagccggcttctggacctcatcacctgggagctagagcctgatgga
gccttggaccgatgcccttgtgccagtggcctcctctgccagccccacag
ccacagcctggtgtatgtgtgcaagccgaccttcgtggggagccgtgacc
aagatggggagatcctgctgcccagagaggtccccgatgagtatgaagtt
ggcagcttcatggaggaggtgcgccaggagctggaggacctggagaggag
cctgactgaagagatggcgctgggggagcctgcggctgccgccgctgcac
tgctgggaggggaagagatttagatctggaccaggctgtgggtagatgtg
caatagaaatagctaatttatttccccaggtgtgtgctttaggcgtgggc
tgaccaggcttcttcctacatcttcttcccagtaagtttcccctctggct
tgacagcatgaggtgttgtgcatttgttcagctcccccaggctgttctcc
aggcttcacagtctggtgcttgggagagtcaggcagggttaaactgcagg
agcagtttgccacccctgtccagattattggctgctttgcctctaccagt
tggcagacagccgtttgttctacatggctttgataattgtttgaggggag
gagatggaaacaatgtggagtctccctctgattggttttggggaaatgtg
gagaagagtgccctgctttgcaaacatcaacctggcaaaaatgcaacaaa
tgaattttccacgcagttctttccatgggcataggtaagctgtgccttca
gctgttgcagatgaaatgttctgttcaccctgcattacatgtgtttattc
atccagcagtgttgctcagctcctacctctgtgccagggcagcattttca
tatccaagatcaattccctctctcagcacagcctggggagggggtcattg
ttctcctcgtccatcagggatctcagaggnctcagagactgcaagctgct
tgcccaagtcacacagctagtgaagaccagagcagtttcatctggttgtg
actctaagctcagtgctctctccactaccccacaccagccttggtgccac
caaaagtgctccccaaaaggaaggagaatgggatttttcttttgaggcat
gcacatctggaattaaggtcaaactaattctcacatccctctaaaagtaa
actactgttaggaacagcagtgttctcacagtgtggggcagccgtccttc
taatgaagacaatgatattgacactgtccctctttggcagttgcattagt
aactttgaaaggtatatgactgagcgtagcatacaggttaacctgcagaa
acagtacttaggtaattgtagggcgaggattataaatgaaatttgcaaaa
tcacttagcagcaactgaagacaattatcaaccacgtggagaaaatcaaa
ccgagcagggctgtgtgaaacatggttgtaatatgcgactgcgaacactg
aactctacgccactccacaaatgatgttttcaggtgtcatggactgttgc
caccatgtattcatccagagttcttaaagtttaaagttgcacatgattgt
ataagcatgctttctttgagttttaaattatgtataaacataagttgcat
ttagaaatcaagcataaatcac
DEFINITION Homo sapiens dickkopf (Xenopus laevis)
homolog 4 (DKK4), mRNA.
ACCESSION XM_032444

SEQ ID NO:35

Agacgacgtgctgagctgccagcttagtggaagctctgctctgggtggag
agcagcctcgctttggtgacgcacagtgctgggaccctccaggagccccg
ggattgaaggatggtggcggccgtcctgctggggctgagctggctctgct
ctcccctgggagctctggtcctggacttcaacaacatcaggagctctgct
gacctgcatggggcccggaagggctcacagtgcctgtctgacacggactg
caataccagaaagttctgcctccagccccgcgatgagaagccgttctgtg
ctacatgtcgtgggttgcggaggaggtgccagcgagatgccatgtgctgc
cctgggacactctgtgtgaacgatgtttgtactacgatggaagatgcaac
cccaatattagaaaggcagcttgatgagcaagatggcacacatgcagaag
gaacaactgggcacccagtccaggaaaaccaacccaaaaggaagccaagt
attaagaaatcacaaggcaggaagggacaagagggagaaagttgtctgag
aacttttgactgtggccctggactttgctgtgctcgtcatttttggacga
aaatttgtaagccagtccttttggagggacaggtctgctccagaagaggg
cataaagacactgctcaagctccagaaatcttccagcgttgcgactgtgg
ccctggactactgtgtcgaagccaattgaccagcaatcggcagcatgctc
gattaagagtatgccaaaaaatagaaaagctataaatatttcaaaataaa
gaagaatccacattgc
DEFINITION Human Frizzled related protein Frzb
precursor (fzrb) mRNA, complete cds
ACCESSION U24163

SEQ ID NO:36

Acggggcctgggcggsaggggcggtggctggagctcggtaaagctcgtgg
gaccccattgggggaatttgatccaaggaagcggtgattgccgggggagg
agaagctcccagatccttgtgtccacttgcagcgggggaggcggagacgc
ggagcgggccttttggcgtccactgcgcggctgcaccctgccccatcctg
ccgggatcatggtctgcggcagcccgggagggatgctgctgctgcgggcc
gggctgcttgccctggctgctctctgcctgctccgggtgcccggggctcg
ggctgcagcctgtgagcccgtccgcatccccctgtgcaagtccctgccct
ggaacatgactaagatgcccaaccacctgcaccacagcactcaggccaac
gccatcctggccatcgagcagttcgaaggtctgctgggcacccactgcag
ccccgatctgctcttcttcctctgtgccatgtacgcgcccatctgcacca
ttgacttccagcacgagcccatcaacccctgtaagtctgtgtgcgagcgg
gcccggcagggctgtgagcccatactcatcaagtaccgccactcgtggcc
ggagaacctggcctgcgaggagctgccagtgtacgacaggggcgtgtgca
tctctcccgaggccatcgttactgcggacggagctgattttcctatggat
tctagtaacggaaactgtagaggggcaagcagtgaacgctgtaaatgtaa
gcctattagagctacacagaagacctatttccggaacaattacaactatg
tcattcgggctaaagttaaagagataaagactaagtgccatgatgtgact
gcagtagtggaggtgaaggagattctaaagtcctctctggtaaacattcc
acgggacactgtcaacctctataccagctctggctgcctctgccctccac
ttaatgttaatgaggaatatatcatcatgggctatgaagatgaggaacgt
tccagattactcttggtggaaggctctatagctgagaagtggaaggatcg
actcggtaaaaaagttaagcgctgggatatgaagcttcgtcatcttggac
tcagtaaaagtgattctagcaatagtgattccactcagagtcagaagtct
ggcaggaactcgaacccccggcaagcacgcaactaaatcccgaaatacaa
aaagtaacacagtggacttcctattaagacttacttgcattgctggacta
gcaaaggaaaattgcactattgcacatcatattctattgtttactataaa
aatcatgtgataactgattattacttctgtttctcttttggtttctgctt
ctctcttctctcaacccctttgtaatggtttgggggcagactcttaagta
tattgtgagttttctatttcactaatcatgagaaaaactgttcttttgca
ataataataaattaaacatgctgtta
DEFINITION Homo sapiens secreted apoptosis related
protein 1 (SARP1) mRNA, partial cds.
ACCESSION AF017986

SEQ ID NO:37

Gaattcgttcagcctggttaagtccaagctggctcattctgctcccccgg
gtcggagccccccggagctgcgcgcgggcttgcagcgcctcgcccgcgct
gtcctcccggtgtcccgcttctccgcgccccagccgccggctgccagctt
ttcggggccccgagtcgcacccagcgaagagagcgggcccgggacaagct
cgaactccggccgcctcgcccttaaccagctccgtccctctaccccctag
gggtcgcgcccacgatgctgcagggccctggctcgctgctgctgctcttc
ctcgcctcgcactgctgcctgggctcggcgcgcgggctcttcctctttgg
ccagcccgacttctcctacaagcgcagcaattgcaagcccatcccggcca
acctgcagctgtgccacggcatcgaataccagaacatgcggctgcccaac
ctgctgggccacgagaccatgaaggaggtgctggagcaggccggcgcttg
gatcccgctggtcatgaagcagtgccacccggacaccaagaagttcctgt
gctcgctcttcgcccccgtctgcctcgatgacctagacgagaccatccag
ccatgccactctcgntgcgtgcaggtgaaggatcgctgcgccccggtcat
gtccgccttcccctggcccgacatgcttgagtgcgaccgtttcccccagg
acaacgacctttgcatccccctcgctagcagcgaccacctcctgccagcc
accgaggaagctccaaaggtatgtgaagcctgcaaaaataaaaatgatga
tgacaacgacataatggaaacgctttgtaaaaatgattttgcactgaaaa
taaaagtgaaggagataacctacatcaaccgt
Cytoplasmic acting components:
DEFINITION Homo sapiens adenomatosis polyposis
coli (APC), mRNA.
ACCESSION XM_043933

SEQ ID NO:38

Aggtccaagggtagccaaggatggctgcagcttcatatgatcagttgtta
aagcaagttgaggcactgaagatggagaactcaaatcttcgacaagagct
agaagataattccaatcatcttacaaaactggaaactgaggcatctaata
tgaaggaagtacttaaacaactacaaggaagtattgaagatgaagctatg
gcttcttctggacagattgatttattagagcgtcttaaagagcttaactt
agatagcagtaatttccctggagtaaaactgcggtcaaaaatgtccctcc
gttcttatggaagccgggaaggatctgtatcaagccgttctggagagtgc
agtcctgttcctatgggttcatttccaagaagagggtttgtaaatggaag
cagagaaagtactggatatttagaagaacttgagaaagagaggtcattgc
ttcttgctgatcttgacaaagaagaaaaggaaaaagactggtattacgct
caacttcagaatctcactaaaagaatagatagtcttcctttaactgaaaa
tttttccttacaaacagatatgaccagaaggcaattggaatatgaagcaa
ggcaaatcagagttgcgatggaagaacaactaggtacctgccaggatatg
gaaaaacgagcacagcgaagaatagccagaattcagcaaatcgaaaagga
catacttcgtatacgacagcttttacagtcccaagcaacagaagcagaga
ggtcatctcagaacaagcatgaaaccggctcacatgatgctgagcggcag
aatgaaggtcaaggagtgggagaaatcaacatggcaacttctggtaatgg
tcagggttcaactacacgaatggaccatgaaacagccagtgttttgagtt
ctagtagcacacactctgcacctcgaaggctgacaagtcatctgggaacc
aaggtggaaatggtgtattcattgttgtcaatgcttggtactcatgataa
ggatgatatgtcgcgaactttgctagctatgtctagctcccaagacagct
gtatatccatgcgacagtctggatgtcttcctctcctcatccagctttta
catggcaatgacaaagactctgtattgttgggaaattcccggggcagtaa
agaggctcgggccagggccagtgcagcactccacaacatcattcactcac
agcctgatgacaagagaggcaggcgtgaaatccgagtccttcatcttttg
gaacagatacgcgcttactgtgaaacctgttgggagtggcaggaagctca
tgaaccaggcatggaccaggacaaaaatccaatgccagctcctgttgaac
atcagatctgtcctgctgtgtgtgttctaatgaaactttcatttgatgaa
gagcatagactagcaatgaatgaactagggggactacaggccattgcaga
attattgcaagtggactgtgaaatgtatgggcttactaatgaccactaca
gtattacactaagacgatatgctggaatggctttgacaaacttgactttt
ggagatgtagccaacaaggctacgctatgctctatgaaaggctgcatgag
agcacttgtggcccaactaaaatctgaaagtgaagacttacagcaggtta
ttgcgagtgttttgaggaatttgtcttggcgagcagatgtaaatagtaaa
aagacgttgcgagaagttggaagtgtgaaagcattgatggaatgtgcttt
agaagttaaaaaggaatcaaccctcaaaagcgtattgagtgccttatgga
atttgtcagcacattgcactgagaataaagctgatatatgtgctgtagat
ggtgcacttgcatttttggttggcactcttacttaccggagccagacaaa
cactttagccattattgaaagtggaggtgggatattacggaatgtgtcca
gcttgatagctacaaatgaggaccacaggcaaatcctaagagagaacaac
tgtctacaaactttattacaacacttaaaatctcatagtttgacaatagt
cagtaatgcatgtggaactttgtggaatctctcagcaagaaatcctaaag
accaggaagcattatgggacatgggggcagttagcatgctcaagaacctc
attcattcaaagcacaaaatgattgctatgggaagtgctgcagctttaag
gaatctcatggcaaataggcctgcgaagtacaaggatgccaatattatgt
ctcctggctcaagcttgccatctcttcatgttaggaaacaaaaagcccta
gaagcagaattagatgctcagcacttatcagaaacttttgacaatataga
caatttaagtcccaaggcatctcatcgtagtaagcagagacacaagcaaa
gtctctatggtgattatgtttttgacaccaatcgacatgatgataatagg
tcagacaattttaatactggcaacatgactgtcctttcaccatatttgaa
tactacagtgttacccagctcctcttcatcaagaggaagcttagatagtt
ctcgttctgaaaaagatagaagtttggagagagaacgcggaattggtcta
ggcaactaccatccagcaacagaaaatccaggaacttcttcaaagcgagg
tttgcagatctccaccactgcagcccagattgccaaagtcatggaagaag
tgtcagccattcatacctctcaggaagacagaagttctgggtctaccact
gaattacattgtgtgacagatgagagaaatgcacttagaagaagctctgc
tgcccatacacattcaaacacttacaatttcactaagtcggaaaattcaa
ataggacatgttctatgccttatgccaaattagaatacaagagatcttca
aatgatagtttaaatagtgtcagtagtagtgatggttatggtaaaagagg
tcaaatgaaaccctcgattgaatcctattctgaagatgatgaaagtaagt
tttgcagttatggtcaatacccagccgacctagcccataaaatacatagt
gcaaatcatatggatgataatgatggagaactagatacaccaataaatta
tagtcttaaatattcagatgagcagttgaactctggaaggcaaagtcctt
cacagaatgaaagatgggcaagacccaaacacataatagaagatgaaata
aaacaaagtgagcaaagacaatcaaggaatcaaagtacaacttatcctgt
ttatactgagagcactgatgataaacacctcaagttccaaccacattttg
gacagcaggaatgtgtttctccatacaggtcacggggagccaatggttca
gaaacaaatcgagtgggttctaatcatggaattaatcaaaatgtaagcca
gtctttgtgtcaagaagatgactatgaagatgataagcctaccaattata
gtgaacgttactctgaagaagaacagcatgaagaagaagagagaccaaca
aattatagcataaaatataatgaagagaaacgtcatgtggatcagcctat
tgattatagtttaaaatatgccacagatattccttcatcacagaaacagt
cattttcattctcaaagagttcatctggacaaagcagtaaaaccgaacat
atgtcttcaagcagtgagaatacgtccacaccttcatctaatgccaagag
gcagaatcagctccatccaagttctgcacagagtagaagtggtcagcctc
aaaaggctgccacttgcaaagtttcttctattaaccaagaaacaatacag
acttattgtgtagaagatactccaatatgtttttcaagatgtagttcatt
atcatctttgtcatcagctgaagatgaaataggatgtaatcagacgacac
aggaagcagattctgctaataccctgcaaatagcagaaataaaagaaaag
attggaactaggtcagctgaagatcctgtgagcgaagttccagcagtgtc
acagcaccctagaaccaaatccagcagactgcagggttctagtttatctt
cagaatcagccaggcacaaagctgttgaattttcttcaggagcgaaatct
ccctccaaaagtggtgctcagacacccaaaagtccacctgaacactatgt
tcaggagaccccactcatgtttagcagatgtacttctgtcagttcacttg
atagttttgagagtcgttcgattgccagctccgttcagagtgaaccatgc
agtggaatggtaagtggcattataagccccagtgatcttccagatagccc
tggacaaaccatgccaccaagcagaagtaaaacacctccaccacctcctc
aaacagctcaaaccaagcgagaagtacctaaaaataaagcacctactgct
gaaaagagagagagtggacctaagcaagctgcagtaaatgctgcagttca
gagggtccaggttcttccagatgctgatactttattacattttgccacgg
aaagtactccagatggattttcttgttcatccagcctgagtgctctgagc
ctcgatgagccatttatacagaaagatgtggaattaagaataatgcctcc
agttcaggaaaatgacaatgggaatgaaacagaatcagagcagcctaaag
aatcaaatgaaaaccaagagaaagaggcagaaaaaactattgattctgaa
aaggacctattagatgattcagatgatgatgatattgaaatactagaaga
atgtattatttctgccatgccaacaaagtcatcacgtaaagcaaaaaagc
cagcccagactgcttcaaaattacctccacctgtggcaaggaaaccaagt
cagctgcctgtgtacaaacttctaccatcacaaaacaggttgcaacccca
aaagcatgttagttttacaccgggggatgatatgccacgggtgtattgtg
ttgaagggacacctataaacttttccacagctacatctctaagtgatcta
acaatcgaatcccctccaaatgagttagctgctggagaaggagttagagg
aggggcacagtcaggtgaatttgaaaaacgagataccattcctacagaag
gcagaagtacagatgaggctcaaggaggaaaaacctcatctgtaaccata
cctgaattggatgacaataaagcagaggaaggtgatattcttgcagaatg
cattaattctgctatgcccaaagggaaaagtcacaagcctttccgtgtga
aaaagataatggaccaggtccagcaagcatctgcgtcttcttctgcaccc
aacaaaaatcagttagatggtaagaaaaagaaaccaacttcaccagtaaa
acctataccacaaaatactgaatataggacacgtgtaagaaaaaatgcag
actcaaaaaataatttaaatgctgagagagttttctcagacaacaaagat
tcaaagaaacagaatttgaaaaataattccaaggtcttcaatgataagct
cccaaataatgaagatagagtcagaggaagttttgcttttgattcacctc
atcattacacgcctattgaaggaactccttactgtttttcacgaaatgat
tctttgagttctctagattttgatgatgatgatgttgacctttccaggga
aaaggctgaattaagaaaggcaaaagaaaataaggaatcagaggctaaag
ttaccagccacacagaactaacctccaaccaacaatcagctaataagaca
caagctattgcaaagcagccaataaatcgaggtcagcctaaacccatact
tcagaaacaatccacttttccccagtcatccaaagacataccagacagag
gggcagcaactgatgaaaagttacagaattttgctattgaaaatactccg
gtttgcttttctcataattcctctctgagttctctcagtgacattgacca
agaaaacaacaataaagaaaatgaacctatcaaagagactgagccccctg
actcacagggagaaccaagtaaacctcaagcatcaggctatgctcctaaa
tcatttcatgttgaagataccccagtttgtttctcaagaaacagttctct
cagttctcttagtattgactctgaagatgacctgttgcaggaatgtataa
gctccgcaatgccaaaaaagaaaaagccttcaagactcaagggtgataat
gaaaaacatagtcccagaaatatgggtggcatattaggtgaagatctgac
acttgatttgaaagatatacagagaccagattcagaacatggtctatccc
ctgattcagaaaattttgattggaaagctattcaggaaggtgcaaattcc
atagtaagtagtttacatcaagctgctgctgctgcatgtttatctagaca
agcttcgtctgattcagattccatcctttccctgaaatcaggaatctctc
tgggatcaccatttcatcttacacctgatcaagaagaaaaaccctttaca
agtaataaaggcccacgaattctaaaaccaggggagaaaagtacattgga
aactaaaaagatagaatctgaaagtaaaggaatcaaaggaggaaaaaaag
tttataaaagtttgattactggaaaagttcgatctaattcagaaatttca
ggccaaatgaaacagccccttcaagcaaacatgccttcaatctctcgagg
caggacaatgattcatattccaggagttcgaaatagctcctcaagtacaa
gtcctgtttctaaaaaaggcccaccccttaagactccagcctccaaaagc
cctagtgaaggtcaaacagccaccacttctcctagaggagccaagccatc
tgtgaaatcagaattaagccctgttgccaggcagacatcccaaataggtg
ggtcaagtaaagcaccttctagatcaggatctagagattcgaccccttca
agacctgcccagcaaccattaagtagacctatacagtctcctggccgaaa
ctcaatttcccctggtagaaatggaataagtcctcctaacaaattatctc
aacttccaaggacatcatcccctagtactgcttcaactaagtcctcaggt
tctggaaaaatgtcatatacatctccaggtagacagatgagccaacagaa
ccttaccaaacaaacaggtttatccaagaatgccagtagtattccaagaa
gtgagtctgcctccaaaggactaaatcagatgaataatggtaatggagcc
aataaaaaggtagaactttctagaatgtcttcaactaaatcaagtggaag
tgaatctgatagatcagaaagacctgtattagtacgccagtcaactttca
tcaaagaagctccaagcccaaccttaagaagaaaattggaggaatctgct
tcatttgaatctctttctccatcatctagaccagcttctcccactaggtc
ccaggcacaaactccagttttaagtccttcccttcctgatatgtctctat
ccacacattcgtctgttcaggctggtggatggcgaaaactcccacctaat
ctcagtcccactatagagtataatgatggaagaccagcaaagcgccatga
tattgcacggtctcattctgaaagtccttctagacttccaatcaataggt
caggaacctggaaacgtgagcacagcaaacattcatcatcccttcctcga
gtaagcacttggagaagaactggaagttcatcttcaattctttctgcttc
atcagaatccagtgaaaaagcaaaaagtgaggatgaaaaacatgtgaact
ctatttcaggaaccaaacaaagtaaagaaaaccaagtatccgcaaaagga
acatggagaaaaataaaagaaaatgaattttctcccacaaatagtacttc
tcagaccgtttcctcaggtgctacaaatggtgctgaatcaaagactctaa
tttatcaaatggcacctgctgtttctaaaacagaggatgtttgggtgaga
attgaggactgtcccattaacaatcctagatctggaagatctcccacagg
taatactcccccggtgattgacagtgtttcagaaaaggcaaatccaaaca
ttaaagattcaaaagataatcaggcaaaacaaaatgtgggtaatggcagt
gttcccatgcgtaccgtgggtttggaaaatcgcctgaactcctttattca
ggtggatgcccctgaccaaaaaggaactgagataaaaccaggacaaaata
atcctgtccctgtatcagagactaatgaaagttctatagtggaacgtacc
ccattcagttctagcagctcaagcaaacacagttcacctagtgggactgt
tgctgccagagtgactccttttaattacaacccaagccctaggaaaagca
gcgcagatagcacttcagctcggccatctcagatcccaactccagtgaat
aacaacacaaagaagcgagattccaaaactgacagcacagaatccagtgg
aacccaaagtcctaagcgccattctgggtcttaccttgtgacatctgttt
aaaagagaggaagaatgaaactaagaaaattctatgttaattacaactgc
tatatagacattttgtttcaaatgaaactttaaaagactgaaaaattttg
taaataggtttgattcttgttagagggtttttgttctggaagccatattt
gatagtatactttgtcttcactggtcttattttgggaggcactcttgatg
gttaggaaaaaaatagtaaagccaagtatgtttgtacagtatgttttaca
tgtatttaaagtagcatcccatcccaacttcctttaattattgcttgtct
taaaataatgaacactacagatagaaaatatgatatattgctgttatcaa
tcatttctagattataaactgactaaacttacatcagggaaaaattggta
tttatgcaaaaaaaaatgtttttgtccttgtgagtccatctaacatcata
attatcatgtggctgtgaaattcacagtaatatggttcccgatgaacaag
tttacccagcctgctttgctttactgcatgaatgaaactgatggttcaat
ttcagaagtaatgattaacagttatgtggtcacatgatgtgcatagagat
agctacagtgtaataatttacactattttgtgctccaaacaaaacaaaaa
tctgtgtaactgtaaaacattgaatgaaactattttacctgaactagatt
ttatctgaaagtaggtagaatttttgctatgctgtaatttgttgtatatt
ctggtatttgaggtgagatggctgctcttttattaatgagacatgaattg
tgtctcaacagaaactaaatgaacatttcagaataaattattgctgtatg
taaactgttactgaaattggtatttgtttgaagggtcttgtttcacattt
gtattaataattgtttaaaatgcctcttttaaaagcttatataaattttt
ttcttcagcttctatgcattaagagtaaaattcctcttactgtaataaaa
acaattgaagaagactgttgccacttaaccattccatgcgttggcactta
tctattcctgaaatttcttttatgtgattagctcatcttgatttttaata
tttttccacttaaacttttttttcttactccactggagctcagtaaaagt
aaattcatgtaatagcaatgcaagcagcctagcacagactaagcattgag
cataataggcccacataatttcctctttcttaatattatagaattctgta
cttgaaattgattcttagacattgcagtctcttcgaggctttacagtgta
aactgtcttgccccttcatcttcttgttgcaactgggtctgacatgaaca
ctttttatcaccctgtatgttagggcaagatctcagcagtgaagtataat
cagcactttgccatgctcagaaaattcaaatcacatggaactttagaggt
agatttaatacgattaagatattcagaagtatattttagaatccctgcct
gttaaggaaactttatttgtggtaggtacagttctggggtacatgttaag
tgtccccttatacagtggagggaagtcttccttcctgaaggaaaataaac
tgacacttattaactaagataatttacttaatatatcttccctgatttgt
tttaaaagatcagagggtgactgatgatacatgcatacatatttgttgaa
taaatgaaaatttatttttagtgataagattcatacactctgtatttggg
gagggaaaacctttttaagcatggtggggcactcagataggagtgaatac
acctacctggtg
DEFINITION Homo sapiens axin (AXIN1), mRNA.
ACCESSION XM_027520

SEQ ID NO:39

Gggcccggggtcccgccaccaccgcgcgcgggacagattgattcactttg
gagctgtaagtactgatgtattagggtgcagcgctcattgttccttgacg
cagagtcccaaaatgaatatccaagagcagggtttccccttggacctcgg
agcaagtttcaccgaagatgctccccgacccccagtgcctggtgaggagg
gagaactggtgtccacagacccgaggcccgccagctacagtttctgctcc
gggaaaggtgttggcattaaaggtgagacttcgacggccactccgaggcg
ctcggatctggacctggggtatgagcctgagggcagtgcctcccccaccc
caccatacttgaagtgggctgagtcactgcattccctgctggatgaccaa
gatgggataagcctgttcaggactttcctgaagcaggagggctgtgccga
cttgctggacttctggtttgcctgcactggcttcaggaagctggagccct
gtgactcgaacgaggagaagaggctgaagctggcgagagccatctaccga
aagtacattcttgataacaatggcatcgtgtcccggcagaccaagccagc
caccaagagcttcataaagggctgcatcatgaagcagctgatcgatcctg
ccatgtttgaccaggcccagaccgaaatccaggccactatggaggaaaac
acctatccctccttccttaagtctgatatttatttggaatatacgaggac
aggctcggagagccccaaagtctgtagtgaccagagctctgggtcaggga
cagggaagggcatatctggatacctgccgaccttaaatgaagatgaggaa
tggaagtgtgaccaggacatggatgaggacgatggcagagacgctgctcc
ccccggaagactccctcagaagctgctcctggagacagctgccccgaggg
tctcctccagtagacggtacagcgaaggcagagagttcaggtatggatcc
tggcgggagccagtcaacccctattatgtcaatgccggctatgccctggc
cccagccaccagtgccaacgacagcgagcagcagagcctgtccagcgatg
cagacaccctgtccctcacggacagcagcgtggatgggatccccccatac
aggatccgtaagcagcaccgcagggagatgcaggagagcgtgcaggtcaa
tgggcgggtgcccctacctcacattcccgtaagtaccgggtgccgaagga
ggtccgcgtggagcctcagaagttcgcggaggagctcatccaccgcctgg
aggctgtgcagcgcacgcgggaggccgaggagaagctggaggagcggctg
aagcgcgtgcgcatggaggaggaaggtgaggacggcgatccatcgtcagg
gcccccagggccgtgtcacaagctgcctcccgcccccgcttggcaccact
tcccgccccgctgtgtggacatgggctgtgccgggctccgggatgcacac
gaggagaaccctgagagcatcctggacgagcacgtacagcgtgtgctgag
gacacctggccgccagtcgcctgggcctggccatcgctccccggacagtg
ggcacgtggccaagatgccagtggcactggggggtgccgcctcggggcac
gggaagcacgtacccaagtcaggggcgaagctggacgcggccggcctgca
ccaccaccgacacgtccaccaccacgtccaccacagcacagcccggccca
aggagcaggtggaggccgaggccacccgcagggcccagagcagcttcgcc
tggggcctggaaccacacagccatggggcaaggtcccgaggctactcaga
gagtgttggcgctgcccccaacgccagtgatggcctcgcccacagtggga
aggtgggcgtggcgtgcaaaagaaatgccaagaaggctgagtcggggaag
agcgccagcaccgaggtgccaggtgcctcggaggatgcggagaagaacca
gaaaatcatgcagtggatcattgagggggaaaaggagatcagcaggcacc
gcaggaccggccacgggtcttcggggacgaggaagccacagccccatgag
aactccagacccttgtcccttgagcacccctgggccggccctcagctccg
gacctccgtgcagccctcccacctcttcatccaagaccccaccatgccac
cccacccagctcccaaccccctaacccagctggaggaggcgcgccgacgt
ctggaggaggaagaaaagagagccagccgagcaccctccaagcagaggta
tgtgcaggaggttatgcggcggggacgcgcctgcgtcaggccagcgtgcg
cgccggtgctgcacgtggtaccagccgtgtcggacatggagctctccgag
acagagacaagatcgcagaggaaggtgggcggcgggagtgcccagccgtg
tgacagcatcgttgtggcgtactacttctgcggggaacccatcccctacc
gcaccctggtgaggggccgcgctgtcaccctgggccagttcaaggagctg
ctgaccaaaaagggcagctacagatactacttcaagaaagtgagcgacga
gtttgactgtggggtggtgtttgaggaggttcgagaggacgaggccgtcc
tgcccgtctttgaggagaagatcatcggcaaagtggagaaggtggactga
taggctggtgggctggccgctgtgccaggcgaggcccttggcgggcacgg
gtgtcacggccaggcagatgacctcgtactcaggagcccgatggggaaca
gtgttgggtgtaccacccatccctgtggtctacccgtgtctagaggcagg
tagggggtccctccaagtggtccacaagcttctgtcctgcccccaaggag
gcagcctggaccactcctcatagcaatacttggagggcccagcccaagtg
aggcagccgaggtccctgctgccagcttcaggtgacccccccccatcccc
cggcacctcccttgggcacgtgtgctgggatctactttccctctgggatt
tgcccacgtacccaggtctgggtggggcccaggcccggatgcagaggcct
gcagggcctctgtcaattgtacgcgccaccgagtgccttcaacacagctt
gtctcttgcctgccactgtgtgaatcggcgacggagcactgcacctgcct
ccagccgccggctgtgcagtcctgggtcctcctttctgagggcccgtgta
aatatgtacatttctcaggctaggccagcaggggctgcccgagtctgttt
ttcatgcgatgacacttgtacaattaattatcttttcaaaggtacttgga
taataatgaaataaaactgtttttgaacctg
DEFINITION Homo sapiens AXIN2 (AXIN2) mRNA,
complete cds.
ACCESSION AF205888

SEQ ID NO:40

Gttcagatgagcccctgctgacttgagagagacagagagaccacgccgat
tgctgagaggaactggaagaagaagagattccagactcagtgggaagagc
tccctcaccatgagtagcgctatgttggtgacttgcctcccggaccccag
cagcagcttccgtgaggatgccccgcggcccccagtgccaggggaagaag
gggagaccccaccgtgtcaccatggtgggcaagggccaggtcaccaaacc
catgtctgtctcttccaacaccaggcggaacgaagatgggttggggagcc
ggaggggcgggcatctccggattccccctctgacccggtggaccaagtcc
ttacactccttattgggcgatcaagacggtgcttacctgttccgaacttt
cctggagagggagaaatgcgtggataccttagacttctggtttgcctgca
atggattcaggcagatgaacctgaaggataccaaaactttacgagtagcc
aaagcgatctacaaaaggtacattgagaacaacagcattgtctccaagca
gctgaagcctgccaccaagacctacataagagatggcatcaagaagcagc
agattgattccatcatgtttgaccaggcgcagaccgagatccagtcggtg
atggaggaaaatgcctaccagatgtttttgacttctgatatatacctcga
atatgtgaggagtgggggagaaaacacagcttacatgagtaatgggggac
tcgggagcctaaaggtcgtgtgtggctatctccccaccttgaatgaagaa
gaggagtggacttgtgccgacttcaagtgcaaactttcgccaaccgtggt
tggcttgtccagcaaaactctgagggccacggcgagtgtgaggtccacgg
aaactgttgacagtggatacaggtccttcaagaggagcgatcctgttaat
ccttatcacataggttctggctatgtctttgcaccagccaccagcgccaa
cgacagtgagatatccagtgatgcgctgacggatgattccatgtccatga
cggacagcagtgtagatggaattcctccttatcgtgtgggcagtaagaaa
cagctccagagagaaatgcatcgcagtgtgaaggccaatggcagagtgtc
tctacctcatttcccgagaacccaccgcctgcccaaggagatgacccccg
tggaacccgccacctttgcagctgagctgatctcgaggctggaaaagctg
aagctggagttggagagccgccacagcctggaggagcgcctgcagcagat
ccgagaggatgaagagagagagggctccgagctcacactcaattcgcggg
agggggcgcccacgcagcaccccctctccctactgccctccggcagctac
gaggaagacccgcagacgatactggacgatcacctgtccagggtcctcaa
gacccctggctgccagtctccgggcgtaggccgctatagccctcgctccc
gctccccggaccaccaccaccaccaccattcgcagtaccactccctgctc
ccgcccggtggcaagctgcctcccgcggccgcctcgccgggcgcctgccc
cctcctcgggggcaaaggctttgtgaccaagcagacgacgaagcatgtcc
accaccactacatccaccaccatgccgtccccaagaccaaggaggagatc
gaggcggaggccacgcagcgggtgcactgcttctgccctgggggcagcga
gtattactgctactcgaaatgcaaaagccactccaaggctccggaaacca
tgcccagcgagcagtttggtgcccaaagcacaaaaaaggcctaccccttg
gagtctgcccgctcgtctccaggcgaacgagccagccggcaccatctgtg
ggggggcaacagcgggcacccccgcaccaccccccgtgcccacctgttca
cccaggaccctgcgatgccttccctgaccccacccaacacgctggctcat
ctggaggaggcctgtcgcaggctagctgaggtgtcgaagcccccaaagca
gcggtgctgtgtggccagtcagcagagggacaggaatcattcggccactg
ttcagacgggagccacacccttctccaatccaagcctggctccagaagat
cacaaagagccaaagaaactggcaggtgtccacgcgctccaggccagtga
gttggttgtcacttactttttctgtggggaagaaattccataccggagga
tgctgaaggctcagagcttgaccctgggccactttaaagagcagctcagc
aaaaagggaaattataggtattacttcaaaaaagcaagcgatgagtttgc
ctgtggagcggtgtttgaggagatctgggaggatgagacggtgctcccga
tgtatgaaggccggattctgggcaaagtggagcggatcgattgagccctg
gggtctggctttggtgaactgttggagcccgaagctcttgtgaactgtct
tggctgtgagcaactgcgacaaaacattttgaaggaaaa
DEFINITION Homo sapiens frequently rearranged in
advanced T-cell lymphomas (FRAT1), mRNA.
ACCESSION XM_050913

SEQ ID NO:41

Attccggctcccgcggctgcaggcgcgcggctagagtgcctggcgggctc
cggcttccgcgtccgccccggccccggtccagacttagtcttcagctccg
cgcccgctccgccgcggcccaccgcgcccgccggcagccgagcccccagc
gacgcccgcacagctccgggtgcccagacagggggccatgccgtccggag
ggaggaggaagaggaagccggcgaggaggcggagggggaggaagaggagg
aggacagcttcctcctactgcagcagtcagtggcgctgggcagctcgggc
gaggtggaccggctggtggcccagatcggcgagacgctgcagctggacgc
ggcgcagcacagcccggcctcgccgtgcgggcccccgggggcgccgctgc
gggccccggggcccctggctgcggcggtgccggcggacaaggccaggtcc
ccggcggtgccgctgctgctgccgcccgcgttggcggagactgtgggccc
ggcgccccctggggtcctgcgctgcgccctgggggaccgcggccgcgtgc
ggggccgcgctgcgccctactgcgtggccgagctcgccacaggccccagc
gcgctgtccccactgccccctcaggccgaccttgatgggcctccgggagc
tggcaagcagggcatcccgcagccgctgtcgggtccgtgccggcgaggat
ggctccggggcgccgccgcctcccgccgcctgcagcagcgacgcgggtcc
caaccagaaacccgcacaggcgacgacgacccgcaccggcttctgcagca
gctagtgctctctggaaacctcatcaaggaggccgtgcgaaggcttcatt
cgcgacggctgcagttacgtgcaaagcttccccaacgcccgctcctggga
cctctgtcggccccggtgcatgaacccccttcgcctcgcagccctcgcgc
ggcctgcagtgaccctggcgcctccgggagggcgcagctcagaactggcg
acggcgttcttgtgcctggcagctaacacgcccggggtggccacagcgcc
agcctcagactggagggcaaggggttcccttgagggctgcagttctactc
aggctggtggagaactctggcttttggaagcgagagtaaaaagctaatga
cgaggaaccgaaaaatcgcgagtgtttcgcgggtaactggggttgagggc
caaaatatttggaatgaaggactttggccctatttaaggcagattttaca
gagcgcacctcaaacgtacaagtcagtaggactccttatttggcgtgacc
cgacctggccgcggagcctgcatttcctcgcagcctctcagtgccctcca
gccccgcgaccatgtggccacaatccacgcttctccggatcgcggtgcgc
cggaaccacggaggatgatgccagttacttgctttaccttttcagggctg
gctcctgatccactttgggggaggagaacatgagtagataatttcagggt
gcagcccaatctgccagacttaaaaaaaccatcttgtgtctttggaggtg
ctgcttaataccaaacatgcggtgccatgaagggaccctttgggggttga
ataggagttaacccctgcgctctctttgcaactgtctctcttctcagagt
ggtgggggaaggctgtacgacacgggtggggaaaggaggtgggggcgggg
agtattgaatggtggtggaagggtagagaggcgcggagtgaaccccacgc
cctgtctaaagtgtattttcagagccggcccgcctctcctcggttcaagg
tcactgtttcctgggcacgcactgggttgcgggacagagtagccaggttc
tgccggtgctcggagaagagcgcagtgttttgcaagtgctggagtctcct
gaggacacgcgcgtcgccgccaccgcgggtgtgggaaagcgcggacgtgc
tgggcggctgtgcttcggtaggcgaccaccgcccctggccgcgctccggg
ctttcacggaaactcccgagaccgggccctgggttcctcctctcctactc
ggctctgcagtcctactcaagcgggtggctctgggatcctgggggcctgg
gttgggggctagggagacgccatgtgatggacactccagggacacacagc
ctagcacagcagcttataatgggctctccggggccatttgcaataacagc
tgcaattccctggatagacgagttgatttcctccctctgcccctccccca
gccatgccagctggcctttgtaagtgcaggaaaccgagtagaaaatgtga
ccctccaaatggagaagctgcaggctttgccattgtgaaccatggtgaag
tgcttggaacatactgttcactcactctaaaggcgctgagactgtgctgt
tgttctcgtttttatagtcaatggcttgttcatcatccagatgtggctac
tgacatatctacacttcgcaccggagtgtctggaattgtggctatcctga
ttataggattttaacttaactgaaatgcctgctttgaataaatgtgttgg
gttttt
DEFINITION Homo sapiens glycogen synthase kinase 3
beta (GSK3B), mRNA.
ACCESSION XM_010970

SEQ ID NO:42

Gaaggaaggaaaaggtgattcgcgaagagagtgatcatgtcagggcggcc
cagaaccacctcctttgcggagagctgcaagccggtgcagcagccttcag
cttttggcagcatgaaagttagcagagacaaggacggcagcaaggtgaca
acagtggtggcaactcctgggcagggtccagacaggccacaagaagtcag
ctatacagacactaaagtgattggaaatggatcatttggtgtggtatatc
aagccaaactttgtgattcaggagaactggtcgccatcaagaaagtattg
caggacaagagatttaagaatcgagagctccagatcatgagaaagctaga
tcactgtaacatagtccgattgcgttatttcttctactccagtggtgaga
agaaagatgaggtctatcttaatctggtgctggactatgttccggaaaca
gtatacagagttgccagacactatagtcgagccaaacagacgctccctgt
gatttatgtcaagttgtatatgtatcagctgttccgaagtttagcctata
tccattcctttggaatctgccatcgggatattaaaccgcagaacctcttg
ttggatcctgatactgctgtattaaaactctgtgactttggaagtgcaaa
gcagctggtccgaggagaacccaatgtttcgtatatctgttctcggtact
atagggcaccagagttgatctttggagccactgattatacctctagtata
gatgtatggtctgctggctgtgtgttggctgagctgttactaggacaacc
aatatttccaggggatagtggtgtggatcagttggtagaaataatcaagg
tcctgggaactccaacaagggagcaaatcagagaaatgaacccaaactac
acagaatttaaattccctcaaattaaggcacatccttggactaaggtctt
ccgaccccgaactccaccggaggcaattgcactgtgtagccgtctgctgg
agtatacaccaactgcccgactaacaccactggaagcttgtgcacattca
ttttttgatgaattacgggacccaaatgtcaaactaccaaatgggcgaga
cacacctgcactcttcaacttcaccactcaagaactgtcaagtaatccac
ctctggctaccatccttattcctcctcatgctcggattcaagcagctgct
tcaacccccacaaatgccacagcagcgtcag
DEFINITION Homo sapiens beta-catenin-interacting
protein ICAT (ICAT), mRNA.
ACCESSION NM_020248

SEQ ID NO:43

Ggccgctcctgctgctgctactgccgccgccgcagcggctgctcgggctg
agcacgccccggaacaggccgccgcgcgctgcgcgccggacccgctgccc
ctgccggcccggccgggtcgggcggcccagggaccgacagacttgacaac
ggtgacagcactggggcggcaccttcctacttctgcccagccacagccct
cccctcacagttgagcacctgtttgcctgaagttaatttccagaagcagg
agtccccagagccaggcagggggatgaaccgcgagggagctcccgggaag
agtccggaggagatgtacattcagcagaaggtccgagtgctgctcatgct
gcggaagatgggatcaaacctgacagccagcgaggaggagttcctgcgca
cctatgcaggggtggtcaacagccagctcagccagctgcctccgcactcc
atcgaccagggtgcagaggacgtggtgatggcgttttccaggtcggagac
ggaagaccggaggcagtagctgcaaagcccttggaacaccctggatgctg
ttgaagggccaagagatctgtgtggctcctgggccggctgaatggcagca
gccccccttgccccacctcccccttccctacccaaccctgccctgcccca
ccccacctcacagctactcagtggggctggcatcaagggagacaccagtg
gtgcgtttataattggcttaaagggatggacttgtgattggctgcaggaa
gaaacttttttattttttaaatcttgaccaacagaaaccttttattttta
tttctgactcttattttttaaaaaatttgcgcctcggtatctggcttccc
tggaagctctccgagctctggtgctttagttaggtcatttttttagaaat
gtgaagaggtctgattggctgcttaaactggaaagggactgtgattggct
ggttaatgggaaacggtttttttctttggctgcaggtgttctgctgatat
caacagcttccctattttgaatgcagaaaacagggtctgggacattagtc
gttatatttgacttgaaaagaaagaaaccaagtgcgctttgcaatattta
ttacacaaagaacttgctgctgccttcacatttggggtttgtgtttgatt
ggctttcgatgcgtgtgtttggtttcccattggttcacctgtgactcctg
ttgccatggattcacccccctctgctgccggctctgggcctgagggtcca
cctggagagtacatttgctttaatgagtgcacctgcctccaccagcaagg
ggaccccgagaaccctgagcagggtccacagctggaaagttgggcccctg
aggagctttgtgtcgtcttgaacgagcagcccagggcctagaggtaaccg
ttaggcgggatttatgtgcactgcctgcatgagctggcaaccagccagcg
tcccttggtgagaaagggattgctgaggcaccgtccaggccccaccggcc
aggccgcgcccagcagaggcgtactacccagctctgtcctcttggccatc
cttctgtgtaccacttcctgaggcctcattttgggggtcatcttggaaag
gggaggagcttctcccagtgtgagaccccaaagactctggaggtcatctg
gcggaggtctctgggagcccagaacccacataaaagccccagcttggctc
acaaggcccaggagacctccagctaaacaccaacccctgacctaccccag
ccaggctcctacctgtctgctgccagcacagtaggtcccggccagctctg
gagttctctcatcggaggcccatgccctccactccactgcctttggaagg
gtctctctccaggtcagcctggaagggacagtatcgtttgtttatgaaat
gccactgggacagctggctgggccttcaccaagcaagtcccttcagactg
gcccttaagccaaactcaggcccagaattgcagttcagaatggcagtcct
ggaggcagggggtgaggggcaggtctagtgttcctgcaccaaacctaagt
ccttccacctgccacccccttccctgggagggaggtggtcctcctatctc
cctggctcactggcaggtgtgggatctggggagagcggctggagaaagat
gcagtcctcaggaagggggccgccaccctcccctatgctggtagatgctg
aggcccctaggtgcccagggccagtgggaccctctcagaaccaaatcttt
cccctttctcggggcttggggctcgggccgtaggggctcctgagtgtcat
gaagtgcacaggagccaaatgaccgagccctggagagccccatggtgggt
aggtggttcgtgctgtgctctggcaccatcagcctgttccagaaggagga
ttcgagcatcaggctaagaccctgtgtcctccaccatgcactcaccccta
gccctggttagctgacagtcagctgtggggaacacagctacaaccctacc
ctggcagggacctgagagcatctcaggaggggcagcgcatgtgtgcatgt
gctgtgtgagtgagcacacccgtgtgcacactcatacacatgtgcacaca
cacgcactctccccgctcaggggcctggaggtctggctgagcccctgggg
aaaggtgagttctttcatctccctcctccaggtcggagtgcctggagtca
ggtgtcgaggccacattgctggctgccccctctttgtagctcctataaag
ggcccacacctggtggatacctggttgagcgtgtggtctctgccccagcc
tgtccttgtcacgatcacaggccttgcttttgtaacaatgatgaccccgg
cctgtctcatcttctgaagaggaaaagtcaaagtgttgctgtggctccat
atttcaactaaaaatatatctgttggagaaagaaattaacaataaagaat
tttcataggttaaaaaaaaaaaanaaaaag
DEFINITION Homo sapiens DVl-binding protein IDAX
mRNA, complete cds.
ACCESSION AF272159

SEQ ID NO:44

Ggcggcaggaccagcatgcaccaccgaaacgactcccagaggctggggaa
agctggctgcccgccagagccgtcgttgcaaatggcaaatactaatttcc
tctccaccttatcccctgaacactgcagacctttggcgggggaatgcatg
aacaagctcaaatgcggcgctgctgaagcagagataatgaatctccccga
gcgcgtggggactttttccgctatcccggctttagggggcatctcattac
ctccaggggtcatcgtcatgacagcccttcactcccccgcagcagcctca
gcagccgtcacagacagtgcgtttcaaattgccaatctggcagactgccc
gcagaatcattcctcctcctcctcgtcctcctcagggggagctggcggag
ccaacccagccaagaagaagaggaaaaggtgtggggtctgcgtgccctgc
aagaggctcatcaactgtggcgtctgcagcagttgcaggaaccgcaaaac
gggacaccagatctgcaaatttagaaaatgtgaagagctaaagaaaaaac
ctggcacttcactagagagaacacctgttcccagcgctgaagcattccga
tggttcttttaaagcagtagtatatcttattttcaaggcatttggaaatg
aagggcaaactaatgtcttgttttaagaaactgcttagtccaccactgaa
gaaaatatccagaaattattttcattttatgtatagggatttcttcaaaa
aaaaaaaaaaa
DEFINITION Homo sapiens orphan nuclear receptor
(PAR1) mRNA, complete cds.
ACCESSION AF084645

SEQ ID NO:45

Cctctgaaggttctagaatcgatagtgaattcgtgggacgggaagaggaa
gcactgcctttacttcagtgggaatctcggcctcagcctgcaagccaagt
gttcacagtgaaaaaagcaagagaataagctaatactcctgtcctgaaca
aggcagcggctccttggtaaagctactccttgatcgatcctttgcaccgg
attgttcaaagtggaccccaggggagaagtcggagcaaagaacttaccac
caagcagtccaagaggcccagaagcaaacctggaggtgagacccaaagaa
agctggaaccatgctgactttgtacactgtgaggacacagagtctgttcc
tggaaagcccagtgtcaacgcagatgaggaagtcggaggtccccaaatct
gccgtgtatgtggggacaaggccactggctatcacttcaatgtcatgaca
tgtgaaggatgcaagggctttttcaggagggccatgaaacgcaacgcccg
gctgaggtgccccttccggaagggcgcctgcgagatcacccggaagaccc
ggcgacagtgccaggcctgccgcctgcgcaagtgcctggagagcggcatg
aagaaggagatgatcatgtccgacgaggccgtggaggagaggcgggcctt
gatcaagcggaagaaaagtgaacggacagggactcagccactgggagtgc
aggggctgacagaggagcagcggatgatgatcagggagctgatggacgct
cagatgaaaacctttgacactaccttctcccatttcaagaatttccggct
gccaggggtgcttagcagtggctgcgagttgccagagtctctgcaggccc
catcgagggaagaagctgccaagtggagccaggtccggaaagatctgtgc
tctttgaaggtctctctgcagctgcggggggaggatggcagtgtctggaa
ctacaaacccccagccgacagtggcgggaaagagatcttctccctgctgc
cccacatggctgacatgtcaacctacatgttcaaaggcatcatcagcttt
gccaaagtcatctcctacttcagggacttgcccatcgaggaccagatctc
cctgctgaagggggccgctttcgagctgtgtcaactgagattcaacacag
tgttcaacgcggagactggaacctgggagtgtggccggctgtcctactgc
ttggaagacactgcaggtggcttccagcaacttctactggagcccatgct
gaaattccactacatgctgaagaagctgcagctgcatgaggaggagtatg
tgctgatgcaggccatctccctcttctccccagaccgcccaggtgtgctg
cagcaccgcgtggtggaccagctgcaggagcaattcgccattactctgaa
gtcctacattgaatgcaatcggccccagcctgctcataggttcttgttcc
tgaagatcatggctatgctcaccgagctccgcagcatcaatgctcagcac
acccagcggctgctgcgcatccaggacatacacccctttgctacgcccct
catgcaggagttgttcggcatcacaggtagctgagcggctgcccttgggt
gacacctccgagaggcagccagacccagagccctctgagccgccactccc
gggccaagacagatggacactgccaagagccgacaatgccctgctggcct
gtctccctagggaattcctgctatgacagctggctagcattcctcaggaa
ggacatgggtgccccccacccccagttcagtctgtagggagtgaagccac
agactcttacgtggagagtgcactgacctgtaggtcaggaccatcagaga
ggcaaggttgccctttccttttaaaaggccctgtggtctggggagaaatc
cctcagatcccactaaagtgtcaaggtgtggaagggaccaagcgaccaag
gataggccatctggggtctatgcccacatacccacgtttgttcgcttcct
gagtcttttcattgctacctctaatagtcctgtctcccacttcccactcg
ttcccctcctcttccgagctgctttgtgggctcaaggcctgtactcatcg
gcaggtgcatgagtatctgtgggagtcctctagagagatgagaagccagg
aggcctgcaccaaatgtcagaagcttggcatgacctcattccggccacat
cattctgtgtctctgcatccatttgaacacattattaagcactgataata
ggtagcctgctgtggggtatacagcattgactcagatatagatcctgagc
tcacagagtttatagttaaaaaaacaaacagaaacacaaacaatttggat
caaaaggagaaaatgataagtgacaaaagcagcacaaggaatttccctgt
gtggatgctgagctgtgatggcaggcactgggtacccaagtgaaggttcc
cgaggacatgagtctgtaggagcaagggcacaaactgcagctgtgagtgc
gtgtgtgtgatttggtgtaggtaggtctgtttgccacttgatggggcctg
ggtttgttcctggggctggaatgctgggtatgctctgtgacaaggctacg
ctgacaatcagttaaacacaccggagaagaaccatttacatgcaccttat
atttctgtgtacacatctattctcaaagctaaagggtatgaaagtgcctg
ccttgtttatagccacttgtgagtaaaaatttttttgcattttcacaaat
tatactttatataaggcattccacacctaagaactagttttgggaaatgt
agccctgggtttaatgtcaaatcaaggcaaaaggaattaaataatgtact
tttggctaaaaaaaaaaaaaaaaaaaaaaaaaaaaaaaaaaaaaaaaaaa
aaaaa
DEFINITION Homo sapiens transforming growth factor
beta-activated kinase-binding protein 1 (TAB1),
mRNA.
ACCESSION XM_010000

SEQ ID NO:46

Cccgcaggggttcctccaagatggcggcgcagaggaggagcttgctgcag
agtgagcagcagccaagctggacagatgacctgcctctctgccacctctc
tggggttggctcagcctccaaccgcagctactctgctgatggcaagggca
ctgagagccacccgccagaggacagctggctcaagttcaggagtgagaac
aactgcttcctgtatggggtcttcaacggctatgatggcaaccgagtgac
caacttcgtggcccagcggctgtccgcagagctcctgctgggccagctga
atgccgagcacgccgaggccgatgtgcggcgtgtgctgctgcaggccttc
gatgtggtggagaggagcttcctggagtccattgacgacgccttggctga
gaaggcaagcctccagtcgcaattgccagagggagtccctcagcaccagc
tgcctcctcagtatcagaagatccttgagagactcaagacgttagagagg
gaaatttcgggaggggccatggccgttgtggcggtccttctcaacaacaa
gctctacgtcgccaatgtcggtacaaaccgtgcacttttatgcaaatcga
cagtggatgggttgcaggtgacacagctgaacgtggaccacaccacagag
aacgaggatgagctcttccgtctttcgcagctgggcttggatgctggaaa
gatcaagcaggtggggatcatctgtgggcaggagagcacccggcggatcg
gggattacaaggttaaatatggctacacggacattgaccttctcagcgct
gccaagtccaaaccaatcatcgcagagccagaaatccatggggcacagcc
gctggatggggtgacgggcttcttggtgctgatgtcggaggggttgtaca
aggccctagaggcagcccatgggcctgggcaggccaaccaggagattgct
gcgatgattgacactgagtttgccaagcagacctccctggacgcagtggc
ccaggccgtcgtggaccgggtgaagcgcatccacagcgacaccttcgcca
gtggtggggagcgtgccaggttctgcccccggcacgaggacatgaccctg
ctagtgaggaactttggctacccgctgggcgaaatgagccagcccacacc
gagcccagccccagctgcaggaggacgagtgtaccctgtgtctgtgccat
actccagcgcccagagcaccagcaagaccagcgtgaccctctcccttgtc
atgccctcccagggccagatggtcaacggggctcacagtgcttccaccct
ggacgaagccacccccaccctcaccaaccaaagcccgaccttaaccctgc
agtccaccaacacgcacacgcagagcagcagctccagctctgacggaggc
ctcttccgctcccggcccgcccactcgctcccgcctggcgaggacggtcg
tgttgagccctatgtggactttgctgagttttaccgcctctggagcgtgg
accatggcgagcagagcgtggtgacagcaccgtagggcagccggagaatg
cagcccaagcagggcctggcatggggcaggacagggtccagccttttcct
aacatctgcctgtgccacaacggccagcaggtgccccatcctctgcccac
agcagactctgtcccatggctctccgggcagtagagtgtgtgagtgcaga
ctggacctgtggttcataccttgtcaccacccgggaagctgaaggccact
tcctcccagatggcctcagccaggaccatcgccctttctcagagcagagg
gccaggtatagaaaccgcagtgggcctgcaagccgcccgagcctccccag
cagcctcctacagagcaggaagaggcgccctgtgaaccctgagtgttgca
ggcccagcagaccctgctgtcccaagcccacccctcctcccaccatcacc
tccctcacctcgggacagtagccctccacttctccagcctctcagccctg
tgctcctgcatccagagtggaacccaggctggtgtccgcatctgtccctg
ggccccacccctggacctgccttggttgtgtcatctgttgtaaacgttca
ggaggaccagggcagcatctggggcctgggatggccacagaaggggcagg
ccaggtggaaaggagccagggggaagtggtctaagagacctggaactgcc
agaggatggcggcctgggcttccccagagccaggcgtgcgggagaggtga
ggactggccccggtgggctgaggcaggggccgctgtcgtcaggcctgagc
cagggtgagctggtgccctctgccggctgagggtagcaagcaggggttgt
gagtcaggctgggggacttgtttgaaagaaagaggagtggaaaatggttc
caggagggaagaggttctttgagacacagtaccctgggaggcataggaga
agggtcgggccagcccagcccagggcctgagttagactatttcccacatg
ttctctgccttcagtggggagggggtgccaccagggctgtcggccaggat
tgccactcctgtttcagaggaagcaggccgagagacttgcaccttggcca
agccacacaatcagtggggcagccagagctcagacctgagccattttgtc
agtatccaggaccccccggattctccacgccctccccatctcccagtctc
cctgccccccatgccccagaccggcccaccagggactagccgctgtcgca
cagcctctggggtgcttggtctctgcgaagtcaaaggcctgacagctctg
tggcctgggaatccattttcctgcggcagagcagggcctggtgtggaacc
agggagctgtgggaagccacagcagaaatggaagaaaaacaggtctcagc
ccagggtcctcgctcactccctcactccccactttgaagccatctctgtt
ctgcaggtgagaggatttaaagtcagtcacaaaggcttgggaacaaaagg
aattc
Nuclear acting components:
DEFINITION H. sapiens mRNA for beta-catenin.
ACCESSION X87838

SEQ ID NO:47

Aagcctctcggtctgtggcagcagcgttggcccggccccgggagcggaga
gcgaggggaggcggagacggaggaaggtctgaggagcagcttcagtcccc
gccgagccgccaccgcaggtcgaggacggtcggactcccgcggcgggagg
agcctgttcccctgagggtatttgaagtataccatacaactgttttgaaa
atccagcgtggacaatggctactcaagctgatttgatggagttggacatg
gccatggaaccagacagaaaagcggctgttagtcactggcagcaacagtc
ttacctggactctggaatccattctggtgccactaccacagctccttctc
tgagtggtaaaggcaatcctgaggaagaggatgtggatacctcccaagtc
ctgtatgagtgggaacagggattttctcagtccttcactcaagaacaagt
agctgatattgatggacagtatgcaatgactcgagctcagagggtacgag
ctgctatgttccctgagacattagatgagggcatgcagatcccatctaca
cagtttgatgctgctcatcccactaatgtccagcgtttggctgaaccatc
acagatgctgaaacatgcagttgtaaacttgattaactatcaagatgatg
cagaacttgccacacgtgcaatccctgaactgacaaaactgctaaatgac
gaggaccaggtggtggttaataaggctgcagttatggtccatcagctttc
taaaaaggaagcttccagacacgctatcatgcgttctcctcagatggtgt
ctgctattgtacgtaccatgcagaatacaaatgatgtagaaacagctcgt
tgtaccgctgggaccttgcataacctttcccatcatcgtgagggcttact
ggccatctttaagtctggaggcattcctgccctggtgaaaatgcttggtt
caccagtggattctgtgttgttttaatgccattacaactctccacaacct
tttattacatcaagaaggagctaaaatggcagtgcgtttagctggtgggc
tgcagaaaatggttgccttgctcaacaaaacaaatgttaaattcttggct
attacgacagactgccttcaaattttagcttatggcaaccaagaaagcaa
gctcatcatactggctagtggtggaccccaagctttagtaaatataatga
ggacctatacttacgaaaaactactgtggaccacaagcagagtgctgaag
gtgctatctgtctgctctagtaataagccggctattgtagaagctggtgg
aatgcaagctttaggacttcacctgacagatccaagtcaacgtcttgttc
agaactgtctttggactctcaggaatctttcagatgctgcaactaaacag
gaagggatggaaggtctccttgggactcttgttcagcttctgggttcaga
tgatataaatgtggtcacctgtgcagctggaattctttctaacctcactt
gcaataattataagaacaagatgatggtctgccaagtgggtggtatagag
gctcttgtgcgtactgtccttcgggctggtgacagggaagacatcactga
gcctgccatctgtgctcttcgtcatctgaccagccgacaccaagaagcag
agatggcccagaatgcagttcgccttcactatggactaccagttgtggtt
aagctcttacacccaccatcccactggcctctgataaaggctactgttgg
attgattcgaaatcttgccctttgtcccgcaaatcatgcacctttgcgtg
agcagggtgccattccacgactagttcagttgcttgttcgtgcacatcag
gatacccagcgccgtacgtccatgggtgggacacagcagcaatttgtgga
gggggtccgcatggaagaaatagttgaaggttgtaccggagcccttcaca
tcctagctcgggatgttcacaaccgaattgttatcagaggactaaatacc
attccattgtttgtgcagctgctttattctcccattgaaaacatccaaag
agtagctgcaggggtcctctgtgaacttgctcaggacaaggaagctgcag
aagctattgaagctgagggagccacagctcctctgacagagttacttcac
tctaggaatgaaggtgtggcgacatatgcagctgctgttttgttccgaat
gtctgaggacaagccacaagattacaagaaacggctttcagttgagctga
ccagctctctcttcagaacagagccaatggcttggaatgagactgctgat
cttggacttgatattggtgcccagggagaaccccttggatatcgccagga
tgatcctagctatcgttcttttcactctggtggatatggccaggatgcct
tgggtatggaccccatgatggaacatgagatgggtggccaccaccctggt
gctgactatccagttgatgggctgccagatctggggcatgcccaggacct
catggatgggctgcctccaggtgacagcaatcagctggcctggtttgata
ctgacctgtaaatcatcctttagctgtattgtctgaacttgcattgtgat
tggcctgtagagttgctgagagggctcgaggggtgggctggtatctcaga
aagtgcctgacacactaaccaagctgagtttcctatgggaacaattgaag
taaactttttgttctggtcctttttggtcgaggagtaacaatacaaatgg
attttgggagtgactcaagaagtgaagaatgcacaagaatggatcacaag
atggaatttagcaaaccctagccttgcttgttaaaatttttttttttttt
tttttaagaatatctgtaatggtactgactttgcttgctttgaagtagct
ctttttttttttttttttttttttttttgcagtaactgttttttaagtct
ctcgtagtgttaagttatagtgaatactgctacagcaatttctattttta
agaattgagtaatggtgtagaacactaattaattcataatcactctaatt
aattgtaatctgaataaagtgtaacaattgtgtagcctttttgtataaaa
tagacaaatagaaaatggtccaattagtttcctttttaatatgcttaaaa
taagcaggtggatctatttcatgtttttgatcaaaaactatttgggatat
gtatgggtagggtaaatcagtaagaggtgttatttggaaccttgttttgg
acagtttaccagttgccttttatcccaaagttgttgtaacctgctgtgat
acgatgcttcaagagaaaatgcggttataaaaaatggttcagaattaaac
ttttaattcatt
DEFINITION Homo sapiens b-TRCP variant E3RS-
IkappaB mRNA, partial cds.
ACCESSION AF101784

SEQ ID NO:48

Atggacccggccgaggcggtgctgcaagagaaggcactcaagtttatgtg
ctctatgcccaggtctctgtggctgggctgctccagcctggcggacagca
tgccttcgctgcgatgcctgtataacccagggactggcgcactcacagct
ttccagaattcctcagagagagaagactgtaataatggcgaaccccctag
gaagataataccagagaagaattcacttagacagacatacaacagctgtg
ccagactctgcttaaaccaagaaacagtatgtttagcaagcactgctatg
aagactgagaattgtgtggccaaaacaaaacttgccaatggcacttccag
tatgattgtgcccaagcaacggaaactctcagcaagctatgaaaaggaaa
aggaactgtgtgtcaaatactttgagcagtggtcagagtcagatcaagtg
gaatttgtggaacatcttatatcccaaatgtgtcattaccaacatgggca
cataaactcgtatcttaaacctatgttgcagagagatttcataactgctc
tgccagctcggggattggatcatatcgctgagaacattctgtcatacctg
gatgccaaatcactatgtgctgctgaacttgtgtgcaaggaatggtaccg
agtgacctctgatggcatgctgtggaagaagcttatcgagagaatggtca
ggacagattctctgtggagaggcctggcagaacgaagaggatggggacag
tatttattcaaaaacaaacctcctgacgggaatgctcctcccaactcttt
ttatagagcactttatcctaaaattatacaagacattgagacaatagaat
ctaattggagatgtggaagacatagtttacagagaattcactgccgaagt
gaaacaagcaaaggagtttactgtttacagtatgatgatcagaaaatagt
aagcggccttcgagacaacacaatcaagatctgggataaaaacacattgg
aatgcaagcgaattctcacaggccatacaggttcagtcctctgtctccag
tatgatgagagagtgatcataacaggatcatcggattccacggtcagagt
gtgggatgtaaatacaggtgaaatgctaaacacgttgattcaccattgtg
aagcagttctgcacttgcgtttcaataatggcatgatggtgacctgctcc
aaagatcgttccattgctgtatgggatatggcctccccaactgacattac
cctccggagggtgctggtcggacaccgagctgctgtcaatgttgtagact
ttgatgacaagtacattgtttctgcatctggggatagaactataaaggta
tggaacacaagtacttgtgaatttgtaaggaccttaaatggacacaaacg
aggcattgcctgtttgcagtacagggacaggctggtagtgagtggctcat
ctgacaacactatcagattatgggacatagaatgtggtgcatgtttacga
gtgttagaaggccatgaggaattggtgcgttgtattcgatttgataacaa
gaggatagtcagtggggcctatgatggaaaaattaaagtgtgggatcttg
tggctgctttggacccccgtgctcctgcagggacactctgtctacggacc
cttgtggagcattccggaagagtttttcgactacagtttgatgaattcca
gattgtcagtagttcacatgatgacacaatcctcatctgggacttcctaa
atgatccagctgcccaagctgaacccccccgttccccttctcgaacatac
acctacatctccagataa
DEFINITION Human CREB-binding protein (CBP) mRNA,
complete cds.
ACCESSION U47741

SEQ ID NO:49

Tgaggaatcaacagccgccatcttgtcgcggacccgaccggggcttcgag
cgcgatctactcggccccgccggtcccgggccccacaaccgcccgcgctc
gctcctctccctcgcagccggcagggcccccgacccccgtccgggccctc
gccggcccggccgcccgtgcccggggctgttttcgcgagcaggtgaaaat
ggctgagaacttgctggacggaccgcccaaccccaaaagagccaaactca
gctcgcccggtttctcggcgaatgacagcacagattttggatcattgttt
gacttggaaaatgatcttcctgatgagctgatacccaatggaggagaatt
aggccttttaaacagtgggaaccttgttccagatgctgcttccaaacata
aacaactgtcggagcttctacgaggaggcagcggctctagtatcaaccca
ggaataggaaatgtgagcgccagcagccccgtgcagcagggcctgggtgg
ccaggctcaagggcagccgaacagtgctaacatggccagcctcagtgcca
tgggcaagagccctctgagccagggagattcttcagcccccagcctgcct
aaacaggcagccagcacctctgggcccacccccgctgcctcccagcactg
aatccgcaagcacaaaagcaagtggggctggcgactagcagccctgccac
gtcacagactggacctggtatctgcatgaatgctaactttaaccagaccc
acccaggcctcctcaatagtaactctggccatagcttaattaatcaggct
tcacaagggcaggcgcaagtcatgaatggatctcttggggctgctggcag
aggaaggggagctggaatgccgtaccctactccagccatgcagggcgcct
cgagcagcgtgctggctgagaccctaacgcaggtttccccgcaaatgact
ggtcacgcgggactgaacaccgcacaggcaggaggcatggccaagatggg
aataactgggaacacaagtccatttggacagccctttagtcaagctggag
ggcagccaatgggagccactggagtgaacccccagttagccagcaaacag
agcatggtcaacagtttgcccaccttccctacagatatcaagaatacttc
agtcaccaacgtgccaaatatgtctcagatgcaaacatcagtgggaattg
tacccacacaagcaattgcaacaggccccactgcagatcctgaaaaacgc
aaactgatacagcagcagctggttctactgcttcatgctcataagtgtca
gagacgagagcaagcaaacggagaggttcgggcctgctcgctcccgcatt
gtcgaaccatgaaaaacgttttgaatcacatgacgcattgtcaggctggg
aaagcctgccaagttgcccattgtgcatcttcacgacaaatcatctctca
ttggaagaactgcacacgacatgactgtcctgtttgcctccctttgaaaa
atgccagtgacaagcgaaaccaacaaaccatcctggggtctccagctagt
ggaattcaaaacacaattggttctgttggcacagggcaacagaatgccac
ttctttaagtaacccaaatcccatagaccccagctccatgcagcgagcct
atgctgctctcggactcccctacatgaaccagccccagacgcagctgcag
cctcaggttcctggccagcaaccagcacagcctcaaacccaccagcagat
gaggactctcaaccccctgggaaataatccaatgaacattccagcaggag
gaataacaacagatcagcagcccccaaacttgatttcagaatcagctctt
ccgacttccctgggggccacaaacccactgatgaacgatggctccaactc
tggtaacattggaaccctcagcactataccaacagcagctcctccttcta
gcaccggtgtaaggaaaggctggcacgaacatgtcactcaggacctgcgg
agccatctagtgcataaactcgtccaagccatcttcccaacacctgatcc
cgcagctctaaaggatcgccgcatggaaaacctggtagcctatgctaaga
aagtggaaggggacatgtacgagtctgccaacagcagggatgaatattat
cacttattagcagagaaaatctacaagatacaaaaagaactagaagaaaa
acggaggtcgcgtttacataaacaaggcatcttggggaaccagccagcct
taccagccccgggggctcagccccctgtgattccacaggcacaacctgtg
agacctccaaatggacccctgtccctgccagtgaatcgcatgcaagtttc
tcaagggatgaattcatttaaccccatgtccttggggaacgtccagttgc
cacaagcacccatgggacctcgtgcagcctccccaatgaaccactctgtc
cagatgaacagcatgggctcagtgccagggatggccatttctccttcccg
aatgcctcagcctccgaacatgatgggtgcacacaccaacaacatgatgg
cccaggcgcccgctcagagccagtttctgccacagaaccagttcccgtca
tccagcggggcgatgagtgtgggcatggggcagccgccagcccaaacagg
cgtgtcacagggacaggtgcctggtgctgctcttcctaaccctctcaaca
tgctggggcctcaggccagccagctaccttgccctccagtgacacagtca
ccactgcacccaacaccgcctcctgcttccacggctgctggcatgccatc
tctccagcacacgacaccacctgggatgactcctccccagccagcagctc
ccactcagccatcaactcctgtgtcgtcttccgggcagactcccaccccg
actcctggctcagtgcccagtgctacccaaacccagagcacccctacagt
ccaggcagcagcccaggcccaggtgaccccgcagcctcaaaccccagttc
agcccccgtctgtggctacccctcagtcatcgcagcaacagccgacgcct
gtgcacgcccagcctcctggcacaccgctttcccaggcagcagccagcat
tgataacagagtccctaccccctcctcggtggccagcgcagaaaccaatt
cccagcagccaggacctgacgtacctgtgctggaaatgaagacggagacc
caagcagaggacactgagcccgatcctggtgaatccaaaggggagcccag
gtctgagatgatggaggaggatttgcaaggagcttcccaagttaaagaag
aaacagacatagcagagcagaaatcagaaccaatggaagtggatgaaaag
aaacctgaagtgaaagtagaagttaaagaggaagaagagagtagcagtaa
cggcacagcctctcagtcaacatctccttcgcagccgcgcaaaaaaatct
ttaaaccagaggagttacgccaggccctcatgccaaccctagaagcactg
tatcgacaggacccagagtcattacctttccggcagcctgtagatcccca
gctcctcggaattccagactattttgacatcgtaaagaatcccatggacc
tctccaccatcaagcggaagctggacacagggcaataccaagagccctgg
cagtacgtggacgacgtctggctcatgttcaacaatgcctggctctataa
tcgcaagacatcccgagtctataagttttgcagtaagcttgcagaggtct
ttgagcaggaaattgaccctgtcatgcagtcccttggatattgctgtgga
cgcaagtatgagttttccccacagactttgtgctgctatgggaagcagct
gtgtaccattcctcgcgatgctgcctactacagctatcagaataggtatc
atttctgtgagaagtgtttcacagagatccagggcgagaatgtgaccctg
ggtgacgacccttcacagccccagacgacaatttcaaaggatcagtttga
aaagaagaaaaatgataccttagaccccgaacctttcgttgattgcaagg
agtgtggccggaagatgcatcagatttgcgttctgcactatgacatcatt
tggccttcaggttttgtgtgcgacaactgcttgaagaaaactggcagacc
tcgaaaagaaaacaaattcagtgctaagaggctgcagaccacaagactgg
gaaaccacttggaagaccgagtgaacaaatttttgcggcgccagaatcac
cctgaagccggggaggtttttgtccgagtggtggccagctcagacaagac
ggtggaggtcaagcccgggatgaagtcacggtttgtggattctggggaaa
tgtctgaatctttcccatatcgaaccaaagctctgtttgcttttgaggaa
attgacggcgtggatgtctgcttttttggaatgcacgtccaagaatacgg
ctctgattgccccctccaaacacgaggcgtgtgtacatttcttatctgga
tagtattcatttcttccggccacgttgcctccgcacagccgtttaccatg
agatccttattggatatttagagtatgtgaagaaattagggtatgtgaca
gggcacatctgggcctgtcctccaagtgaaggagatgattacatcttcca
ttgccacccacctgatcaaaaaatacccaagccaaaacgactgcaggagt
ggtacaaaaagatgctggacaaggcgtttgcagagcggatcatccatgac
tacaaggatattttcaaacaagcaactgaagacaggctcaccagtgccaa
ggaactgccctattttgaaggtgatttctggcccaatgtgttagaagaga
gcattaaggaactagaacaagaagaagaggagaggaaaaaggaagagagc
actgcagccagtgaaaccactgagggcagtcagggcgacagcaagaatgc
caagaagaagaacaacaagaaaaccaacaagaacaaaagcagcatcagcc
gcgccaacaagaagaagcccagcatgcccaacgtgtccaatgacctgtcc
cagaagctgtatgccaccatggagaagcacaaggaggtcttcttcgtgat
ccacctgcacgctgggcctgtcatcaacaccctgccccccatcgtcgacc
ccgaccccctgctcagctgtgacctcatggatgggcgcgacgccttcctc
accctcgccagagacaagcactgggagttctcctccttgcgccgctccaa
gtggtccacgctctgcatgctggtggagctgcacacccagggccaggacc
gctttgtctacacctgcaacgagtgcaagcaccacgtggagacgcgctgg
cactgcactgtgtgcgaggactacgacctctgcatcaactgctataacac
gaagagccatgcccataagatggtgaagtgggggctgggcctggatgacg
agggcagcagccagggcgagccacagtcaaagagcccccaggagtcacgc
cgggtgagcatccagcgctgcatccagtcgctggtgcacgcgtgccagtg
ccgcaacgccaactgctcgctgccatcctgccagaagatgaagcgggtgg
tgcagcacaccaagggctgcaaacgcaagaccaacgggggctgcccggtg
tgcaagcagctcatcgccctctgctgctaccacgccaagcactgccaaga
aaacaaatgccccgtgcccttctgcctcaacatcaaacacaagctccgcc
agcagcagatccagcaccgcctgcagcaggcccagctcatgcgccggcgg
atggccaccatgaacacccgcaacgtgcctcagcagagtctgccttctcc
tacctcagcaccgcccgggacccccacacagcagcccagcacaccccaga
cgccgcagccccctgcccagccccaaccctcacccgtgagcatgtcacca
gctggcttccccagcgtggcccggactcagccccccaccacggtgtccac
agggaagcctaccagccaggtgccggcccccccacccccggcccagcccc
ctcctgcagcggtggaagcggctcggcagatcgagcgtgaggcccagcag
cagcagcacctgtaccgggtgaacatcaacaacagcatgcccccaggacg
cacgggcatggggaccccggggagccagatggcccccgtgagcctgaatg
tgccccgacccaaccaggtgagcgggcccgtcatgcccagcatgcctccc
gggcagtggcagcaggcgccccttccccagcagcagcccatgccaggctt
gcccaggcctgtgatatccatgcaggcccaggcggccgtggctgggcccc
ggatgcccagcgtgcagccacccaggagcatctcacccagcgctctgcaa
gacctgctgcggaccctgaagtcgcccagctcccctcagcagcaacagca
ggtgctgaacattctcaaatcaaacccgcagctaatggcagctttcatca
aacagcgcacagccaagtacgtggccaatcagcccggcatgcagccccag
cctggcctccagtcccagcccggcatgcaaccccagcctggcatgcacca
gcagcccagcctgcagaacctgaatgccatgcaggctggcgtgccgcggc
ccggtgtgcctccacagcagcaggcgatgggaggcctgaacccccagggc
caggccttgaacatcatgaacccaggacacaaccccaacatggcgagtat
gaatccacagtaccgagaaatgttacggaggcagctgctgcagcagcagc
agcaacagcagcagcaacaacagcagcaacagcagcagcagcaagggagt
gccggcatggctgggggcatggcggggcacggccagttccagcagcctca
aggacccggaggctacccaccggcatgcagcagcagcagcgcatgcagca
gcatctccccctccagggcagctccatgggccagatggcggctcagatgg
gacagcttggccagatggggcagccggggctgggggcagacagcaccccc
aacatccagcaagccctgcagcagcggattctgcagcaacagcagatgaa
gcagcagattgggtccccaggccagccgaaccccatgagcccccagcaac
acatgctctcaggacagccacaggcctcgcatctccctggccagcagatc
gccacgtcccttagtaaccaggtgcggtctccagcccctgtccagtctcc
acggccccagtcccagcctccacattccagcccgtcaccacggatacagc
cccagccttcgccacaccacgtctcaccccagactggttccccccacccc
ggactcgcagtcaccatggccagctccatagatcagggacacttggggaa
ccccgaacagagtgcaatgctcccccagctgaacacccccagcaggagtg
cgctgtccagcgaactgtccctggtcggggacaccacgggggacacgcta
gagaagtttgtggagggcttgtagcattgtgagagcatcaccttttccct
ttcatgttcttggaccttttgtactgaaaatccaggcatctaggttcttt
ttattcctagatggaactgcgacttccgagccatggaagggtggattgat
gtttaaagaaacaatacaaagaatatatttttttgttaaaaaccagttga
tttaaatatctggtctctctctttggtttttttttggcgggggggtgggg
ggggttcttttttttccgttttgtttttgtttggggggaggggggttttg
tttggattctttttgtcgtcattgctggtgactcatgcctttttttaacg
ggaaaaacaagttcattatattcatattttttatttgtattttcaagact
ttaaacatttatgtttaaaagtaagaagaaaaataatattcagaactgat
tcctgaaataatgcaagcttataatgtatcccgataactttgtgatgttt
cgggaagatttttttctatagtgaactctgtgggcgtctcccagtattac
cctggatgataggaattgactccggcgtgcacacacgtacacacccacac
acatctatctatacataatggctgaagccaaacttgtcttgcagatgtag
aaattgttgctttgtttctctgataaaactggttttagacaaaaaatagg
gatgatcactcttagaccatgctaatgttactagagaagaagccttcttt
tctttcttctatgtgaaacttgaaatgaggaaaagcaattctagtgtaaa
tcatgcaagcgctctaattcctataaatacgaaactcgagaagattcaat
cactgtatagaatggtaaaataccaactcatttcttatatcatattgtta
aataaactgtgtgcaacagacaaaaagggtggtccttcttgaattcatgt
acatggtattaacacttagtgttcggggttttttgttatgaaaatgctgt
tttcaacattgtatttggactatgcatgtgttttttccccattgtatata
aagtaccgcttaaaattgatataaattactgaggtttttaacatgtattc
tgttctttaagatcccctgtaagaatgtttaaggtttttatttatttata
tatattttttggtctgttctttgtaaaaaaaaaaaaaaaaa.
DEFINITION Homo sapiens C-terminal binding protein
1 (CTBP1), mRNA.
ACCESSION NM_001328

SEQ ID NO:50

Cgcgcagcgcgccggagtggtcggggcccgcggccgctcgcgcctctcga
tgggcagctcgcacttgctcaacaagggcctgccgcttggcgtccgacct
ccgatcatgaacgggcccctgcacccgcggcccctggtggcattgctgga
tggccgggactgcacagtggagatgcccatcctgaaggacgtggccactg
tggccttctgcgacgcgcagtccacgcaggagatccatgagaaggtcctg
aacgaggctgtgggggccctgatgtaccacaccatcactctcaccaggga
ggacctggagaagttcaaagccctccgcatcatcgtccggattggcagtg
gttttgacaacatcgacatcaagtcggccggggatttaggcattgccgtc
tgcaacgtgcccgcggcgtctgtggaggagacggccgactcgacgctgtg
ccacatcctgaacctgtaccggcgggccacctggctgcaccaggcgctgc
gggagggcacacgagtccagagcgtcgagcagatccgcgaggtggcgtcc
ggcgctgccaggatccgcggggagaccttgggcatcatcggacttggtcg
cgtggggcaggcagtggcgctgcgggccaaggccttcggcttcaacgtgc
tcttctacgacccttacttgtcggatggcgtggagcgggcgctggggctg
cagcgtgtcagcaccctgcaggacctgctgctcttccacagcgactgcgt
gaccctgcactgcggcctcaacgagcacaaccaccacctcatcaacgact
tcaccgtcaagcagatgagacaaggggccttcctggtgaacacagcccgg
ggtggcctggtggatgagaaggcgctggcccaggccctgaaggagggccg
gatccgcggcgcggccctggatgtgcacgagtcggaacccttcagcttta
gccagggccctctgaaggatgcacccaacctcatctgcaccccccatgct
gcatggtacagcgagcaggcatccatcgagatgcgagaggaggcggcacg
ggagatccgcagagccatcacaggccggatcccagacagcctgaagaact
gtgtcaacaaggaccatctgacagccgccacccactgggccagcatggac
cccgccgtcgtgcaccctgagctcaatggggctgcctataggtaccctcc
gggcgtggtgggcgtggcccccactggcatcccagctgctgtggaaggta
tcgtccccagcgccatgtccctgtcccacggcctgccccctgtggcccac
ccgccccacgccccttctcctggccaaaccgtcaagcccgaggcggatag
agaccacgccagtgaccagttgtagcccgggaggagctctccagcctcgg
cgcctggggcagcgggcccggaaaccctcgaccagagtgtgtgagagcat
gtgtgtggtggcccctggcactgcagagactggtccgggctgtcaggagg
gcgggagggcgcagcgctgggcctcgtgtcgcttgtcgtccgtcctgtgg
gcgctctgccctgtgtccttcgcgttcctcgttaagcagaagaagtcagt
agttattctcccatgaacgttcttgtctgtgtacagtttttagaacatta
caaaggatctgtttgcttagctgtcaacaaaaagaaaacctgaaggagca
tttggaagtcaatttgaggtttttttttttggttttttttttttgtattt
tggaacgtgccccagaatgaggcagttggcaaacttctcaggacaatgaa
tcttcccgtttttctttttatgccacacagtgcattgttttttctacctg
cttgtcttatttttagcataatttagaaaaacaaaacaaaggctgttttt
cctaattttggcatgaacccccccttgttccaaaatgaagacggcatcat
cacgaagcagctccaaaaggaaaagcttggcaggtgccctcgtcctgggg
acgtggagggtggcacggtccccgcctgcaccagtgccgtcctgctgatg
tggtaggctagcaatattttggttaaaatcatgtttgtggccgaacgggc
ccctgcacccg
DEFINITION Homo sapiens HMG-box containing protein
1 (HBP1), mRNA.
ACCESSION XM_027193

SEQ ID NO:51

Agcaccataacatggtgtgggaagtgaagacaaatcagatgcctaatgca
gtacagaaactcctgttggtgatggacaagagagcctcaggaatgaatga
ctcattggagttgctgcagtgtaatgagaatttgccatcttcacctggat
ataactcctgtgatgaacacatggagcttgatgaccttcctgaacttcag
gcagttcaaagtgatcctacccaatctggcatgtaccagctgagttcaga
tgtttcacatcaagaatacccaagatcatcttggaaccaaaatacctcag
acataccagaaactacttaccgtgaaaatgaggtggactggctaacagaa
ttggcaaatatcgcgaccagtccacaaagtccactgatgcagtgctcatt
ttacaatagatcatctcctgtacacatcatagccactagcaaaagtttac
attcctatgcacgccctccaccagtgtcctcttcttcgaagagtgaacca
gccttccctcatcaccattggaaggaggaaacaccagtaagacacgaaag
ggcaaatagtgagtcagaatctggcattttctgcatgtcctccctgtcag
atgatgatgatttgggatggtgcaattcctggccttcaactgtctggcac
tgttttttgaaaggcacacgactgtgctttcataagggaagcaataagga
atggcaagatgttgaagattttgctagagctgaaggctgtgataatgagg
aagatcttcaaatgggcattcacaagggctatggttctgatggtctaaag
ttgttatcacatgaagaaagtgtatcatttggcgagtctgtactgaagtt
gacttttgatcctggtacagtagaagatggtttacttaccgtagagtgta
agctggaccaccctttctatgttaaaaataaaggttggtcatcattttat
ccaagcttgactgtggtacagcatggcattccatgttgtgaagttcatat
tggcgatgtatgtctacctcctggacaccccgatgccattaattttgatg
attcaggtgtttttgatacatttaaaagctatgacttcacacctatggat
tcttctgcagtttatgtgttaagtagtatggctcgccagcgtcgtgcatc
tttgtcttgtggaggacctggtggtcaagactttgcaagatctggattca
gtaaaaactgtggctcacctggatcatcacagctctcttccaattctttg
tatgctaaagctgtcaaaaaccacagctcagggactgtgagtgccacttc
tcctaataagtgcaaaagaccaatgaatgccttcatgctttttgccaaaa
aatacagagttgaatatactcagatgtatccagggaaagataacagagcc
ataagtgtgatccttggtgacaggtggaagaaaatgaagaatgaagagag
aagaatgtacacattagaagcaaaggctttggctgaagaacagaaacgtt
taaatcctgactgttggaagaggaaaagaaccaattcaggctcacaacaa
cattaaaccaggatgcttatgttcttaagtctatatttgcatatacattg
actcttgatggaaagacttaagaagatcaaggtctcaccatttgtcctca
attcgtgtgaccataagatactgatagcattgagtcttgaaatgatttaa
taatatgagtgaggatttgctttctccattagagcattaagctaaaacta
tcaacattttaaaccaaattgccttatttttcttccaaacttcatatatg
tctatcaggtaataataggcttgaaaattgatatcctgtggtgctaaagt
acagtagaaagagaggagaagtgtatacatgttttattttaaattgtacg
aaaggggaatttaaaaaatatgtaactgctgtttatacattggctcctta
ctgcttattaatctgtattgtacacatgatgaaatgaagcagaagctggg
agtcggcctttcctctagtaaccaccacatggctcagcatctgtgccaaa
cataggcgctcctagtctggtcagtgccaagaggctaccagaacatgggg
caggtggctggtgttggtgtcccagcctaagagccacctgctgcagttac
catggcatgctgagttgatgcaccaggtggcagcagccatccgttattat
ttccaatggagacctagcccaggccaaggtaaagttagttaatagcattg
ggatatagtcactgtaatggtgctattaacaaacagtcaacaccattgta
ttttttaacttcgtgttctgtatctcctcagccatgtatcttaaatatat
tttgtcatcataatctttatggtgggggcagactttgcacttactgcagt
gcaacacttgcactttaattttcctccaactgtctaaaattagagcaaat
acattggcaatacagctgcttttgctctgagctacaatcatggcttttca
tgttacttaccaagtggtgtttctggttaggaatcacagctgtaaaattg
atttcagttcatcacacttcttcatgatgttgcccctaaattttgcacac
tatattcttgtatattatttcaaataaatggaaaaa
DEFINITION Homo sapiens lymphoid enhancer factor-1
(LEF1) mRNA, complete cds.
ACCESSION AF288571

SEQ ID NO:52

Aagatctaaaaacggacatctccaccgtgggtggctcctttttctttttc
tttttttcccacccttcaggaagtggacgtttcgttatcttctgatcctt
gcaccttcttttggggaacggggcccttctgcccagatcccctctctttc
tcggaaaacaaactactaagtcggcatccggggtaactacagtggagagg
gtttccgcggagacgcgccgccggaccctcctctgcactttggggaggcg
tgctccctccagaaccggcgttctccgcgcgcaaatcccggcgacgcggg
gtcgcggggtggccgccggggcagcctcgtctagcgcgcgccgcgcagac
gcccccggagtcgccagctaccgcagccctcgccgcccagtgcccttcgg
cctcggggcgggcgcctgcgtcggtctccgcgaagcgggaaagcgcggcg
gccgccgggattcgggcgccgcggcagctgctccggctgccggccggcgg
ccccgcgctcgcccgccccgcttccgcccgctgtcctgctgcacgaaccc
ttccaactctcctttcctcccccacccttgagttacccctctgtctttcc
tgctgttgcgcgggtgctcccacagcggagcggagattacagagccgccg
ggatgccccaactctccggaggaggtggcggcggcgggggggacccggaa
ctctgcgccacggacgagatgatccccttcaaggacgagggcgatcctca
gaaggaaaagatcttcgccgagatcagtcatcccgaagaggaaggcgatt
tagctgacatcaagtcttccttggtgaacgagtctgaaatcatcccggcc
agcaacggacacgaggtggccagacaagcacaaacctctcaggagcccta
ccacgacaaggccagagaacaccccgatgacggaaagcatccagatggag
gcctctacaacaagggaccctcctactcgagttattccgggtacataatg
atgccaaatatgaataacgacccatacatgtcaaatggatctctttctcc
acccatcccgagaacatcaaataaagtgcccgtggtgcagccatcccatg
cggtccatcctctcacccccctcatcacttacagtgacgagcacttttct
ccaggatcacacccgtcacacatcccatcagatgtcaactccaaacaagg
catgtccagacatcctccagctcctgatatccctactttttatcccttgt
ctccgggtggtgttggacagatcaccccacctcttggctggcaaggtcag
cctgtatatcccatcacgggtggattcaggcaaccctacccatcctcact
gtcagtcgacacttccatgtccaggttttcccatcatatgattcccggtc
ctcctggtccccacacaactggcatccctcatccagctattgtaacacct
caggtcaaacaggaacatccccacactgacagtgacctaatgcacgtgaa
gcctcagcatgaacagagaaaggagcaggagccaaaaagacctcacatta
agaagcctctgaatgcttttatgttatacatgaaagaaatgagagcgaat
gtcgttgctgagtgtactctaaaagaaagtgcagctatcaaccagattct
tggcagaaggtggcatgccctctcccgtgaagagcaggctaaatattatg
aattagcacggaaagaaagacagctacatatgcagctttatccaggctgg
tctgcaagagacaattatggtaagaaaaagaagaggaagagagagaaact
acaggaatctgcatcaggtacaggtccaagaatgacagctgcctacatct
gaaacatggtggaaaacgaagctcattcccaacgtgcaaagccaaggcag
cgaccccaggacctcttctggagatggaagcttgttgaaaacccagactg
tctccacggcctgcccagtcgacgccaaaggaacactgacatcaatttta
ccctgaggtcactgctagagacgctgatccataaagacaatcactgccaa
cccctctttcgtctactgcaagagccaagttccaaaataaagcataaaaa
ggttttttaaaaggaaatgtaaaagcacatgagaatgctagcaggctgtg
gggcagctgagcagcttttctccccccatatctgcgtgcacttcccagag
catcttgcatccaaacctgtaacctttcggcaaggacggtaacttggctg
catttgcctgtcatgcgcaactggagccagcaaccagctatccatcagca
ccccagtggaggagttcatggaagagttccctctttgtttctgcttcatt
tttctttcttttcttttctcctaaagcttttatttaacagtgcaaaagga
tcgtttttttttgcttttttaaacttgaatttttttaatttacacttttt
agttttaattttcttgtatattttgctagctatgagctttaaataaaatt
gaaagttctggaaaagtttgaaataatgacataaaaagaagccttctttt
tctgagacagcttgtctggtaagtggcttctctgtgaattgcctgtaaca
catagtggcttctccgcccttgtaaggtgttcagtagagctaaataaatg
taatagccaaaccccactctgttggtagcaattggcagccctatttcagt
ttattttttcttctgttttcttcttttctttttttaaacagtaaacctta
acagatgcgttcagcagactggtttgcagtgaattttcatttctttcctt
atcacccccttgttgtaaaaagcccagcacttgaattgttattactttaa
atgttctgtatttgtatctgtttttattagccaattagtgggattttatg
ccagttgttaaaatgagcattatgtacccattttttaaaaaagcaagcac
agcctttgccaaaactgtcatcctaacgtttgtcattccagtttgagtta
atgtgctgagcatttttttaaaagaagctttgtaataaaacatttttaaa
aattgtcatttaaaaaaaaaaaaaaaaaa
DEFINITION Homo sapiens nemo-like kinase mRNA,
complete cds.
ACCESSION AF197898

SEQ ID NO:53

Tttcccttttttttccgtttggcaacccacacccttccacacacgctcac
ccgaaaattaaacaccaagatcctctaacttgtttggattgactgatgaa
gacataaagctctatgttttttgaggtggagtgagtggtttttcttcatt
tttaaatggccaaatgacagcttgacccagtttgctttccaatcaaaggg
catttattttgaatgtctctttgtggcgcaagagccaacgcaaaaatgat
ggcggcttacaatggcggtacatctgcagcagcagcaggtcaccaccacc
accatcaccaccaccttccacacctccctcctcctcacctgcaccaccac
caccaccctcaacaccatcttcatccggggtcggctgccgctgtacaccc
tgtacagcagcacacctcttcggcagctgcggcagccgcagcagcggctg
cagctgcagccatgttaaaccctgggcaacaacagccatatttcccatca
ccggcaccggggcaggctcctggaccagctgcagcagccccagctcaggt
acaggctgccgcagctgctacagttaaggcgcaccatcatcagcactcgc
atcatccacagcagcagctggatattgagccggatagacctattggatat
ggagcctttggtgttgtctggtcagtaacagatccaagagatggaaagag
agtagcgctcaaaaagatgcccaacgtcttccagaatctggtctcttgca
aaagggtcttccgggaattgaagatgttgtgtttttttaagcatgataat
gtactctctgcccttgacatactccaacctccacacattgactattttga
agaaatatatgttgtcacagaattgatgcagagtgacctacataaaatta
tcgtctctcctcaaccactcagctcagatcatgtcaaagtttttctttat
cagattttgcgaggtttgaaatatctccattcagctggcattttacatcg
agacattaagccagggaatctccttgtgaacagcaactgtgttctaaaga
tttgtgattttggattggccagagtggaagaattagatgaatcccgtcat
atgactcaggaagttgttactcagtattatcgggctccagaaatcctgat
gggcagccgtcattacagcaatgctattgacatctggtctgtgggatgta
tctttgcagaactactaggacgaagaatattgtttcaggcacagagtccc
attcagcagttggatttgatcacggatctgttgggcacaccatcactgga
agcaatgaggacagcttgtgaaggcgctaaggcacatatactcaggggtc
ctcataaacagccatctcttcctgtactctataccctgtctagccaggct
acacatgaagctgttcatctcctttgcaggatgttggtctttgatccatc
caaaagaatatccgctaaggatgccttagcccacccctacctagatgaag
ggcgactacgatatcacacatgtatgtgtaaatgttgcttttccacctcc
actggaagagtttataccagtgactttgagcctgtcaccaatcccaaatt
tgatgacactttcgagaagaacctcagttctgtccgacaggttaaagaaa
ttattcatcagttcattttggaacagcagaaaggaaacagagtgcctctc
tgcatcaaccctcagtctgctgcttttaagagctttattagttccactgt
tgctcagccatctgagatgcccccatctcctctggtgtgggagtgatggt
ggaagataatgtactactgaagatgtaatgtagctttccactggagtctg
ggatttgcaattctggaggttaatcatgcttgtactgtaattttactaat
gaagttttaaattaacaaccactacttgtatgatatgaataatatttaga
aatgttactagacttttaatcttgtaaagtggttgtgcttttagaagaaa
aatattttacccagagttgcacatgttttatgaatttagtgcagctgtta
tggctcacctcagaacaaaagagaattgaaccaaatttgggagtttgggg
ttttatgttttgtttttcttttctaaaatgaagtgagattgttcacacac
acacacacacacacacacacacacaaacacaaaggacagtcatacatttt
gatatttgagccattcctaaagatttggggttttctaaaactaaagaatc
taggaaccttgcctgcgaccaatcatggagccacgtgagctgatcgtggc
tgcacctggggggagggtagggaggaggggcatgccacctaatgatcaag
ccctataattagcttctcattagagccgtgatggtgatgtgtgctgtcta
aaatccaatgttgtgggtagagagaatgagtttgtgactaggagagacta
aacttttgttttccttacccagtataaatatatatatatatatttaatct
atttttattagaagtttttctgctctttcttacataaaagaaccccaagc
atgcatctttcatgtgtgtaaataattcatttctgggctaatttcaaaag
aatcccaatattgctgtatagaaagagaactagcttgcacattttaggtc
tgtgaaattttgtgagacttttcctgcactggacagtaaaaaaataataa
aagacaaaaacaaatttaaaaaaaaaa
DEFINITION Homo sapiens Pontin52 mRNA, complete
cds.
ACCESSION AF099084

SEQ ID NO:54

Cgggcgggcgcactgtcctagctgctggttttccacgctggttttagctc
ccggcgtctgcaaaatgaagattgaggaggtgaagagcactacgaagacg
cagcgcatcgcctcccacagccacgtgaaagggctggggctggacgagag
cggcttggccaagcaggcggcctcagggcttgtgggccaggagaacgcgc
gagaggcatgtggcgtcatagtagaattaatcaaaagcaagaaaatggct
ggaagagctgtcttgttggcaggacctcctggaactggcaagacagctct
ggctctggctattgctcaggagctgggtagtaaggtccccttctgcccaa
tggtggggagtgaagtttactcaactgagatcaagaagacagaggtgctg
atggagaacttccgcagggccattgggctgcgaataaaggagaccaagga
agtttatgaaggtgaagtcacagagctaactccgtgtgagacagagaatc
ccatgggaggatatggcaaaaccattagccatgtgatcataggactcaaa
acagccaaaggaaccaaacagttgaaactggaccccagcatttttgaaag
tttgcagaaagagcgagtagaagctggagatgtgatttacattgaagcca
acagtggggccgtgaagaggcagggcaggtgtgatacctatgccacagaa
ttcgaccttgaagctgaagagtatgtccccttgccaaaaggggatgtgca
caaaaagaaagaaatcatccaagatgtgaccttgcatgacttggatgtgg
ctaatgcgcggccccaggggggacaagatatcctgtccatgatgggccag
ctaatgaagccaaagaagacagaaatcacagacaaacttcgaggggagat
taataaggtggtgaacaagtacatcgaccagggcattgctgagctggtcc
cgggtgtgctgtttgttgatgaggtccacatgctggacattgagtgcttc
acctacctgcaccgcgccctggagtcttctatcgctcccatcgtcatctt
tgcatccaaccgaggcaactgtgtcatcagaggcactgaggacatcacat
cccctcacggcatccctcttgaccttctggaccgagtgatgataatccgg
accatgctgtatactccacaggaaatgaaacagatcattaaaatccgtgc
ccagacggaaggaatcaacatcagtgaggaggcactgaaccacctggggg
agattggcaccaagaccacactgaggtactcagtgcagctgctgaccccg
gccaacttgcttgctaaaatcaacgggaaggacagcattgagaaagagca
tgtcgaagagatcagtgaacttttctatgatgccaagtcctccgccaaaa
tcctggctgaccagcaggataagtacatgaagtgagatggctgaggtttt
cagcagcaagagactccccaggtgtgcctggcctgggtccagcctgtggg
cgcttgcccctgggcttggggctgccgtccccactcaggcgtgggctgca
gcgctgtcagttcagtgtggaaagcatttctttttaagttatcgtaactg
ttcctgtggttgctttgaaagaacccttccttacctggtgtgttttctat
aaatcttcataggttattttgatcttctctctctctctctaagtttttta
aaaataaacttttcagaacagtaaaaaaaaaaaaaa
DEFINITION Homo sapiens Reptin52 mRNA, complete
cds.
ACCESSION AF124607

SEQ ID NO:55

Gcggccgcgtcgacgagcatcatggcaaccgttacagccacaaccaaagt
cccggagatccgtgatgtaacaaggattgagcgaatcggtgcccactccc
acatccggggactggggctggacgatgccttggagcctcggcaggcttcg
caaggcatggtgggtcagctggcggcacggcgggcggctggcgtggtgct
ggagatgatccgggaagggaagattgccggtcgggcagtccttattgctg
gccagccgggcacggggagacggccatcgccatgggcatggcgcaggccc
tgggccctgacacgccattcacagccatcgccggcagtgaaatcttctcc
ctggagatgagcaagaccgaggcgctgacgcaggccttccggcggtccat
cggcgttcgcatcaaggaggagacggagatcatcgaaggggaggtggtgg
agatccagattgatcgaccagcaacagggacgggctccaaggtgggcaaa
ctgaccctcaagaccacagagatggagaccatctacgacctgggcaccaa
gatgattgagtccctgaccaaggacaaggtccaggccggggacgtgatca
ccatcgacaaggcgacgggcaagatctccaagctgggccgctccttcaca
cgcgcccgcgactacgacgctatgggctcccagaccaagttcgtgcagtg
cccagatggggagctccagaaacgcaaggaggtggtgcacaccgtgtccc
tgcacgagatcgacgtcatcaactctcgcacccagggcttcctggcgctc
ttctcaggtgacacaggggagatcaagtcagaagtccgtgagcagatcaa
tgccaaggtggctgagtggcgcgaggagggcaaggcggagatcatccctg
gagtgctgttcatcgacgaggtccacatgctggacatcgagagcttctcc
ttcctcaaccgggccctggagagtgacatggcgcctgtcctgatcatggc
caccaaccgtggcatcacgcgaatccggggcaccagctaccagagccctc
acggcatccccatagacctgctggaccggctgcttatcgtctccaccacc
ccctacagcgagaaagacacgaagcagatcctccgcatccggtgcgagga
agaagatgtggagatgagtgaggacgcctacacggtgctgacccgcatcg
ggctggagacgtcactgcgctacgccatccagctcatcacagctgccagc
ttggtgtgccggaaacgcaagggtacagaagtgcaggtggatgacatcaa
gcgggtctactcactcttcctggacgagtcccgctccacgcagtacatga
aggagtaccaggacgccttcctcttcaacgaactcaaaggcgagaccatg
gacacctcctgagttggatgtcatcccccgaccccaccctgttttccacc
agagttctgacactgtgactctgtataaaatggttgggaagctgcaaaaa
aaaaaaaaaaaaaaaaa
Wnt target genes:
DEFINITION Homo sapiens achaete-scute complex
(Drosophila) homolog-like 1 (ASCL1), mRNA.
ACCESSION NM_004316

SEQ ID NO:56

Cccgagacccggcgcaagagagcgcagccttagtaggagaggaacgcgag
acgcggcagagcgcgttcagcactgacttttgctgctgcttctgcttttt
tttttcttagaaacaagaaggcgccagcggcagcctcacacgcgagcgcc
acgcgaggctcccgaagccaacccgcgaagggaggaggggagggaggagg
aggcggcgtgcagggaggagaaaaagcattttcaccttttttgctcccac
tctaagaagtctcccggggattttgtatatattttttaacttccgtcagg
gctcccgcttcatatttccttttctttccctctctgttcctgcacccaag
ttctctctgtgtccccctcgcgggccccgcacctcgcgtcccggatcgct
ctgattccgcgactccttggccgccgctgcgcatggaaagctctgccaag
atggagagcggcggcgccggccagcagccccagccgcagccccagcagcc
cttcctgccgcccgcagcctgtttctttgccacggccgcagccgcggcgg
ccgcagccgccgcagcggcagcgcagagcgcgcagcagcagcagcagcag
cagcagcagcagcagcagcagcaggcgccgcagctgagaccggcggccga
cggccagccctcagggggcggtcacaagtcagcgcccaagcaagtcaagc
gacagcgctcgtcttcgcccgaactgatgcgctgcaaacgccggctcaac
ttcagcggctttggctacagcctgccgcagcagcagccggccgccgtggc
gcgccgcaacgagcgcgagcgcaaccgcgtcaagttggtcaacctgggct
ttgccacccttcgggagcacgtccccaacggcgcggccaacaagaagatg
agtaaggtggagacactgcgctcggcggtcgagtacatccgcgcgctgca
gcagctgctggacgagcatgacgcggtgagcgccgccttccaggcaggcg
tcctgtcgcccaccatctcccccaactactccaacgacttgaactccatg
gccggctcgccggtctcatcctactcgtcggacgagggctcttacgaccc
gctcagccccgaggagcaggagcttctcgacttcaccaactggttctgag
gggctcggcctggtcaggccctggtgcgaatggactttggaagcagggtg
atcgcacaacctgcatctttagtgctttcttgtcagtggcgttgggaggg
ggagaaaaggaaaagaaaaaaaaagaagaagaagaagaaaagagaagaag
aaaaaaacgaaaacagtcaaccaaccccatcgccaactaagcgaggcatg
cctgagagacatggctttcagaaaacgggaagcgctcagaacagtatctt
tgcactccaatcattcacggagatatgaagagcaactgggacctgagtca
atgcgcaaaatgcagcttgtgtgcaaaagcagtgggctcctggcagaagg
gagcagcacacgcgttatagtaactcccatcacctctaacacgcacagct
gaaagttcttgctcgggtcccttcacctccccgccctttcttagagtgca
gttcttagccctctagaaacgagttggtgtctttc
DEFINITION Homo sapiens fibroblast growth factor 5
(FGF5), mRNA.
ACCESSION XM_054589

SEQ ID NO:58

Tctcttcccctctccccttctcttccccgaggctatgtccacccggtgcg
gcgaggcgggcagagccagaggcacgcagccgcacaggggctacagagcc
cagaatcagccctacaagatgcacttaggacccccgcggctggaagaatg
agcttgtccttcctcctcctcctcttcttcagccacctgatcctcagcgc
ctgggctcacggggagaagcgtctcgcccccaaagggcaacccggacccg
ctgccactgataggaaccctagaggctccagcagcagacagagcagcagt
agcgctatgtcttcctcttctgcctcctcctcccccgcagcttctctggg
cagccaaggaagtggcttggagcagagcagtttccagtggagcccctcgg
ggcgccggaccggcagcctctactgcagagtgggcatcggtttccatctg
cagatctacccggatggcaaagtcaatggatcccacgaagccaatatgtt
aagccaagttcacagatgactgcaagttcagggagcgttttcaagaaaat
agctataatacctatgcctcagcaatacatagaactgaaaaaacagggcg
ggagtggtatgtggccctgaataaaagaggaaaagccaaacgagggtgca
gcccccgggttaaaccccagcatatctctacccattttctgccaagattc
aagcagtcggagcagccagaactttctttcacggttactgttcctgaaaa
gaaaaagccacctagccctatcaagccaaagattcccctttctgcacctc
ggaaaaataccaactcagtgaaatacagactcaagtttcgctttggataa
tattcctcttggccttgtgagaaaccattctttcccctcaggagtttcta
taggtgtcttcagagttctgaagaaaaattactggaccagcttcagctat
acttacactgtattgaagtcacgtcatttgtttcaatgtgactgaaacaa
aatgttttttgataggaaggaaactggaattctttgtactaatacaggga
gcacactccttcagttcagcaagacataaagccttttgctttatgcttga
gggatatttagaactttgtattttcggaaagttaaataacagggactacg
tatttttctgacttttacagattaacctgaaagaacatacatgatacatt
tttatttttggtttccaaagaatattttgatgcagataaaatattttgtt
aacttttgtttttttttgtttgttttcttaaaagtacctctgcattgagc
atattttcttacttttattattttaattaatatgacataagcaatcattt
tatgctgtttatgaattataaatgtgtttatagctcattaatatggaaat
cttttacatttttcctattcactgcacttttttattgtttttatttctag
ccatacctcagataatatgtttagttttacattttaaaatgtttaaattc
tctttcacagcaccaaaggctcagcttggatttgtgtgtatgtgtatgtc
aattcatgacattatgtggaatcctaaacctttggtggctgggatatgat
gggttagaagcaaggagaaaatataaggactttttgatggaattaaatgt
gggaggtaaggaaaaggatttagaggtaaaagtacactaagtttgcaaca
tttattgagatctaagtctgtcttgccttcatttctctttttatctcccc
cttgccctcattcttgaacagctggaggaatacattttattctgtccatg
aagcatacactatgaaattcaagtgcttaaaaatacttctatgactctct
gctatcccactgtatagatccacagggagcaaacacttagaaatgataga
gaactgaaggagatcaatggtttaacagttatccatgccaagtcccattg
tcagaaatattcttattactcagtcaaacactctttgagcttcccttcct
aaaggtaaccaatccagtgaatagatgtgcccttttataaggaaacttct
gatgtttattaaaaaaactggccttttgatagaggtaacttaatttggga
atttgttgtgttgaaatggcatttaatttcaacctaaatactgactgctg
gacataaatcacagaaaatttaacttaagaaaatttacaaaatttattct
caggtaatcattttaataaagttctgcaaaatacacgtttatcttacatt
cagaaatgtggcaaaaaaggcatagctaaaggctaaacatatggctttag
tagtaacaaaagggttcatagaaacttcatggtttgcatttaaacatgtt
taaagtgtacttataaactatttttttcttaaagcaaactatgatttatt
ttggtgcacaaatacaaagtggaaacttaccaaaattgaactagctacca
tataagcagattgctttaatttgatgggaaaatagtacacacatatatat
aacaaataatatattaaaaaacccatccatcaactaaaacattatatgta
tacatcagtatagtgttttattataaagccaattatctgattaagcattc
tttccactgaatgcataatgtttaaatagcataaaatgaaatgctacaaa
aattgaactaatttatactttaaagtatttctgggttaaatgaaacaatg
aaattttttagtatgttcaactctcatccaaatggcatatgaccctgttt
acacagcctaaagctaaaaatattactctagtttattctaatctattgtt
aagtattgtgcactgtataccaagttcttagggcacatgaaaaattttag
ctgccaaacaggaactagtaaacatatgttcctaataagtgaagggaaag
ataataatgatggtcaacaataagccacgtcaatgcataagttgtatagg
ctaaatgttgcttgtaggctacattaaactcaaatgtaatagtttatctt
atactcctggtttgatttgattagcatattaacgtgaaagtaggatagct
actaaatatatattatgcaagtcaggaatcattaatttcaaaatttaaag
ccatgctaaaattaaaaagaaaatattaaattacacaattacacttgtct
ttactggccatacaaaatgattttttttttttttttgagacagagtcttg
ctctgtcaccaggctggagtgcagtggcatgatctcggctcactgcaacc
tccaactccctggtttaagggattctcctgcctcagcctcccaagtagct
gggattacagactcatgccaccacgccagctaatttttgtatttttagta
gagacggggtttcaccatgttggtcaggatggtctcaatcctggcctctt
gatagtcctgacctcatgatctgcccacctcggcctccccaaagtgctgg
gattacaggtacaatgatgtataattaatgcttagtgaagcataaagtta
cctacatcaattaattaaatgaacttatgtacagaaaacatgtataaata
taagtctatactaatgcttacaactttctaagagggttcttgcttatgta
gctttttattattttaagtaactagaaccaccaaatatcaaataaaatta
tttggttatggttatgttcatctaaacacaacaataacttttatattaat
atttaggagtctattttgtctataggtgacaaacatctccagactaacat
gtcagttttatcaattatattatgtttaattatttaagatttctttatgt
ggaacatctatagagataaatagaaattttcaataagatgtagtaacact
gtgatttatctttcaagagtctctcttcacttccttctaaagagactaat
ttgagagtacaggtgcatattaattttcttggttctttcagctgaattat
attggtccagaagttcaaaatcatgtgacaataataagggatactgacag
aagttatttccaagtttgtgtatatattataaaaattacatatataaaac
taaggcttttatttctgttatttttaagcttttatttcttgtagctaaaa
ataaaacatcataaatctggtaggtaaatttcttattaaatcaatcttga
aatagaaaatgtaataactttcttaccattaacattttttacccttccat
agaagggagggaataaatcatgacttatcccattttcaataacaaaacga
aactatggcactaaccaaaaacttgcattctggcataatttttacagttg
cagagaattgtttctgggctcattaaaaaaagtagtattgcagacattgc
tgcaatgggaagcagacaataacttcttaaaggaattctacacctccttt
aagatttacttaattgctacatctaaattctgataatttaaaatccattt
taggtgataaaattttttaaaagttttgaaggaaacctctggataaatgg
acaaggcctaatttttttttgtagtcaatccaactgtactggccaatttt
tgaaataagattatatgattaggtattagcagagacaaagagttacctcc
tccatcttactctgccctatttgaaagtctcaggggagaaaagggaacaa
gatgctgatccaacctgagtggagtcaggtgaggcatctttacatctaag
aatttttttttaaattttattattattatacttcaagttctagggtacat
gtccacaatgcacatgtctgtcacacatgcacacatgtgccatgctggtg
tgctgcacccaccaacctgtcatccagcattaggtatatctcctaatgct
atccctcccctctccacccaccccacagcaggccccggtatgtgatgttc
cccttcgtgtgtccatgtgttcttattgttcaattcccacctatgagtga
gaatatgtggtgtttggtttttggtccttgcaatagtttgctgagaatga
tggtttccagcttcatccatgtccctacaaagaacatgaactcatcattt
ttgatggctgcatagtattccatggtgtatatgtgccacattttcatcca
gtctatcattgttggacatttgggttggttccagtctttgctattgtgaa
tagtgctgcaataaacatatgtgtg
DEFINITION Homo sapiens muscle segment homeobox 1
(MSX1) gene, complete cds.
ACCESSION AF426432

SEQ ID NO:59

Tgcgtgcccccaggcccagcgcgcctccgggcgagtccccaggagcgcgg
cccaatggatcgctccgggcccgccccctcgcgcgctgattggccgccgc
cccgctggcctcgccttattagcaagttctctggggagccgcggtagggc
ccggagccggcgagtgctcccgggaactctgcctgcgcggcggcagcgac
cggaggccaggcccagcacgccggagctggcctgctggggaggggcggga
ggcgcgcgcgggagggtccgcccggccagggccccgggcgctcgcagagg
ccggccgcgctcccagcccgcccggagcccatgcccggcggctggccagt
gctgcggcagaagggggggcccggctctgcatggccccggctgctgacat
gacttctttgccactcggtgtcaaagtggaggactccgccttcggcaagc
cggcggggggaggcgcgggccaggcccccagcgccgccgcggccacggca
gccgccatgggcgcggacgaggagggggccaagcccaaagtgtccccttc
gctcctgcccttcagcgtggaggcgctcatggccgaccacaggaagccgg
gggccaaggagagcgccctggcgccctccgagggcgtgcaggcggcgggt
ggctcggcgcagccactgggcgtcccgccggggtcgctgggagccccgga
cgcgccctcttcgccgcggccgctcggccatttctcggtggggggactcc
tcaagctgccagaagatgcgctcgtcaaagccgagagccccgagaagccc
gagaggaccccgtggatgcagagcccccgcttctccccgccgccggccag
gcggctgagccccccagcctgcaccctccgcaaacacaagacgaaccgta
agccgcggacgcccttcaccaccgcgcagctgctggcgctggagcgcaag
ttccgccagaagcagtacctgtccatcgccgagcgcgcggagttctccag
ctcgctcagcctcactgagacgcaggtgaagatatggttccagaaccgcc
gcgccaaggcaaagagactacaagaggcagagctggagaagctgaagatg
gccgccaagcccatgctgccaccggctgccttcggcctctccttccctct
cggcggccccgcagctgtagcggccgcggcgggtgcctcgctctacggtg
cctctggccccttccagcgcgccgcgctgcctgtggcgcccgtgggactc
tacacggcccatgtgggctacagcatgtaccacctgacatagagggtccc
aggtcgcccacctgtgggccagccgattcctccagccctggtgctgtacc
cccggacgtgctcccctgctcggcaccgccagccgccttccctttaaccc
tcacactgctccagtttcacctctttgctccctgagttcactctccgaag
tctgatccctgccaaaaagtggctggaagagtcccttagtactcttctag
catttagagatctaccctctcgagttaaaagatggggaaactgagggcag
agaggttaacagatttatctaaggtccccagcagaattgacagtgtgaac
agagctagaggccatgtctcctgcatagtttttccctgtcctgacaccag
gcaagaaagcgcagagaaatcggtgtctgacgattttggaatgagaacaa
tctcaaaaaaaaaaaaaaaaaaaaaaaaaagaaaagagaaaaaaaagact
agccaccaggaagatgaatcctagcttcttccattggaaaatttaaagac
aagttcaacaacaaaacatttgctctggggggcagggaaaacacagatgt
gttgcaaaggtaggttgaaggga
DEFINITION Homo sapiens neurogenin 1 (NEUROG1),
mRNA.
ACCESSION NM_006161

SEQ ID NO:60

Atgccagcccgccttgagacctgcatctccgacctcgactgcgccagcag
cagcggcagtgacctatccggcttcctcaccgacgaggaagactgtgcca
gactccaacaggcagcctccgcttcggggccgcccgcgccggcccgcagg
agcgcgcccaatatctcccgggcgtctgaggttccaggggcacaggacga
cgagcaggagaggcggcggcgccgcggccggacgcgggtccgctccgagg
cgctgctgcactcgctgcgcaggagccggcgcgtcaaggccaacgatccg
cgagcgcaaccgcatgcacaacttgaacgcggccctggacgcactgcgca
gcggctgccctcgttccccgacgacaccaagctcaccaaaatcgagacgc
tgcgcttcgcctacaactacatctgggctctggccgagacactgcgcctg
gcggatcaagggctgcccggaggcggtgcccgggagcgcctcctgccgcc
gcagtgcgtcccctgcctgcccggtcccccaagccccgccagcgacgcgg
agtcctggggctcaggtgccgccgccgcctccccgctctctgaccccagt
agcccagccgcctccgaagacttcacctaccgccccggcgaccctgtttt
ctccttcccaagcctgcccaaagacttgctccacacaacgccctgtttca
ttccttaccactag
DEFINITION Homo sapiens neurogenin 2 gene, partial
cds.
ACCESSION AF303002

SEQ ID NO:61

Ggcctcccccgccttggcggccctgaccccgctgtcatccagcgccgacg
aagaagaggaggaggagccgggcgcgtcaggcggggcgcgtcggcagcgc
ggggctgaggccgggcagggggcgcggggcggcgtggctgcgggtgcgga
gggctgccggcccgcacggctgctgggtctggtacacgattgcaaacggc
gcccttcccgggcgcgggccgtctcccgaggcgccaagacggccgagacg
gtgcagcgcatcaagaagacccgtagactgaaggccaacaaccgcgagcg
aaaccgcatgcacaacctcaacgcggcactggacgcgctgcgcgaggtgc
tccccacttccccgaggacgccaagctcaccaagatcgagaccctgcgct
tcgcccacaactacatctgggcactcaccgagaccctgcgcctggcggat
cactgcgggggcggcggcgggggcctgccgggggcgctcttctccgaggc
agtgttgctgagcccgggaggagccagcgccgccctgagcagcagcggag
acagcccctcgcccgcctccacgtggagttgcaccaacagccccgcgccg
tcctcctccgtgtcctccaattccacctccccctacagctgcactttatc
gcccgccagcccggccgggtcagacatggactattggcagcccccacctc
ccgacaagcaccgctatgcacctcacctccccatagccagggaugtatct
ag
DEFINITION Homo sapiens neurogenin 3 (NEUROG3),
mRNA.
ACCESSION XM_005744

SEQ ID NO:62

Cctcggaccccattctctcttcttttctcctttggggctggggcaactcc
caggcgggggcgcctgcagctcagctgaacttggcgaccagaagcccgct
gagctccccacggccctcgctgctcatcgctctctattctttgcgccggt
agaaaggatgacgcctcaaccctcgggtgcgcccactgtccaagtgaccc
gtgagacggagcggtccttccccagagcctcggaagacgaagtgacctgc
cccacgtccgccccgcccagccccactcgcacacgggggaactgcgcaga
ggcggaagagggaggctgccgaggggccccgaggaagctccgggcacggc
gcgggggacgcagccggcctaagagcgagttggcactgagcaagcagcga
cggagtcggcgaaagaaggccaacgaccgcgagcgcaatcgaatgcacaa
cctcaactcggcactggacgccctgcgcggtgtcctgcccaccttcccag
acgacgcgaagctcaccaagatcgagacgctgcgcttcgcccacaactac
atctgggcgctgactcaaacgctgcgcatagcggaccacagcttgtacgc
gctggagccgccggcgccgcactgcggggagctgggcagcccaggcgtcc
cccggggactgggggtccctctactccccagtctcccaggctggcagcct
gagtcccgccgcgtcgctggaggagcgacccgggctgctgggggccacct
tttccgcctgcttgagcccaggcagtctggctttctcagattttctgtga
aaggacctgtctgtcgctgggctgtgggtgctaagggtaagggagaggga
gggagccgggagccgtagagggtggccgacggcggcggccctcaaaagca
cttgttccttctgcttctccctggctgacccctggccggcccaggcctcc
acgggggcggcaggctgggttcattccccggccctccgagccgcgccaac
gcacgcaacccttgctgctgcccgcgcgaagtgggcattgcaaagtgcgc
tcattttaggcctcctctctgccaccaccccataatctcattcaaagaat
actagaatggtagcactacccggccggagccgcccaccgtcttgggtcgc
cctaccctcactca
DEFINITION Homo sapiens phosphatase and tensin
homolog (mutated in multiple Advanced cancers 1)
(PTEN), mRNA.
ACCESSION NM_000314

SEQ ID NO:63

Cctcccctcgcccggcgcggtcccgtccgcctctcgctcgcctcccgcct
cccctcggtcttccgaggcgcccgggctcccggcgcggcggcggaggggg
cgggcaggccggcgggcggtgatgtggcaggactctttatgcgctgcggc
aggatacgcgctcggcgctgggacgcgactgcgctcagttctctcctctc
ggaagctgcagccatgatggaagtttgagagttgagccgctgtgaggcga
ggccgggctcaggcgagggagatgagagacggcggcggccgcggcccgga
gcccctctcagcgcctgtgagcagccgcgggggcagcgccctcggggagc
cggccggcctgcggcggcggcagcggcggcgtttctcgcctcctcttcgt
cttttctaaccgtgcagcctcttcctcggcttctcctgaaagggaaggtg
gaagccgtgggctcgggcgggagccggctgaggcgcggcggcggcggcgg
cggcacctcccgctcctggagcgggggggagaagcggcggcggcggcggc
cgcggcggctgcagctccagggagggggtctgagtcgcctgtcaccattt
ccagggctgggaacgccggagagttggtctctccccttctactgcctcca
acacggcggcggcggcggcggcacatccagggacccgggccggttttaaa
cctcccgtccgccgccgccgcaccccccgtggcccgggctccggaggccg
ccggcggaggcagccgttcggaggattattcgtcttctccccattccgct
gccgccgctgccaggcctctggctgctgaggagaagcaggcccagtcgct
gcaaccatccagcagccgccgcagcagccattacccggctgcggtccaga
gccaagcggcggcagagcgaggggcatcagctaccgccaagtccagagcc
atttccatcctgcagaagaagccccgccaccagcagcttctgccatctct
ctcctcctttttcttcagccacaggctcccagacatgacagccatcatca
aagagatcgttagcagaaaaaaggagatatcaagaggatggattcgactt
agacttgacctatatttatccaaacattattgctatgggatttcctgcag
aaagacttgaaggcgtatacaggaacaatattgatgatgtagtaaggttt
ttggattcaaagcataaaaaccattacaagatatacaatctttgtgctga
aagacattatgacaccgccaaatttaattgcagagttgcacaatatcctt
ttgaagaccataacccaccacagctagaacttatcaaacccttttgtgaa
gatcttgaccaatggctaagtgaagatgacaatcatgttgcagcaattca
ctgtaaagctggaaagggacgaactggtgtaatgatatgtgcatatttat
tacatcggggcaaatttttaaaggcacaagaggccctagatttctatggg
gaagtaaggaccagagacaaaaagggagtaactattcccagtcagaggcg
ctatgtgtattattatagctacctgttaaagaatcatctggattatagac
cagtggcactgttgtttcacaagatgatgtttgaaactattccaatgttc
agtggcggaacttgcaatcctcagtttgtggtctgccagctaaaggtgaa
gatatattcctccaattcaggacccacacgacgggaagacaagttcatgt
actttgagttccctcagccgttacctgtgtgtggtgatatcaaagtagag
ttcttccacaacagaacaagatgctaaaaaaggacaaaatgtttcacttt
tgggtaaatacattcttcataccaggaccagaggaaacctcagaaaaagt
agaaaatggaagtctatgtgatcaagaaatcgatagcatttgcagtatag
agcgtgcagataatgacaaggaatatctagtacttactttaacaaaaaat
gatcttgacaaagcaaataaagacaaagccaaccgatacttttctccaaa
ttttaaggtgaagctgtacttcacaaaaacagtagaggagccgtcaaatc
cagaggctagcagttcaacttctgtaacaccagatgttagtgacaatgaa
cctgatcattatagatattctgacaccactgactctgatccagagaatga
accttttgatgaagatcagcatacacaaattacaaaagtctgaatttttt
tttatcaagagggataaaacaccatgaaaataaacttgaataaactgaaa
atggacctttttttttttaatggcaataggacattgtgtcagattaccag
ttataggaacaattctcttttcctgaccaatcttgttttaccctatacat
ccacagggttttgacacttgttgtccagttgaaaaaaggttgtgtagctg
tgtcatgtatataccttttgtgtcaaaaggacatttaaaattcaattagg
attaataaagatggcactttcccgttttattccagttttataaaaagtgg
agacagactgatgtgtatacgtaggaattttttccttttgtgttctgtca
ccaactgaagtggctaaagagctttgtgatatactggttcacatcctacc
cctttgcacttgtggcaacagataagtttgcagttggctaagagaggttt
ccgaaaggttttgctaccattctaatgcatgtattcgggttagggcaatg
gaggggaatgctcagaaaggaaataattttatgctggactctggaccata
taccatctccagctatttacacacacctttctttagcatgctacagttat
taatctggacattcgaggaattggccgctgtcactgcttgttgtttgcgc
atttttttttaaagcatattggtgctagaaaaggcagctaaaggaagtga
atctgtattggggtacaggaatgaaccttctgcaacatcttaagatccac
aaatgaagggatataaaaataatgtcataggtaagaaacacagcaacaat
gacttaaccatataaatgtggaggctatcaacaaagaatgggcttgaaca
ttataaaaattgacaatgatttattaaatatgttttctcaattgtaaaaa
aaaaa

TABLE 2
Polycomb Group.

EED

SEQ ID NO:64

>gi|14523051|ref|NM_003797.1|Homo sapiens
embryonic ectoderm development (EED), Mrna
GAGGGAAGTGTCGACTGCGCCGGCGGGAACAGACATGCCTGCGGCCAAGA
AGCAGAAGCTGAGCAGTGACGAGAACAGCAATCCAGAACTCTCTGGAGAC
GAGAATGATGACGCTGTCAGTATAGAAAGTGGTACAAACACTGAACGCCC
TGATACACCTACAAACACGCCAAATGCACCTGGAAGGAAAAGTTGGGGAA
AGGGAAAATGGAAGTCAAAGAAATGCAAATATTCTTTCAAATGTGTAAAT
AGTCTCAAGGAAGATCATAACCAACCATTGTTTGGAGTTCAGTTTAACTG
GCACAGTAAAGAAGGAGATCCATTAGTGTTTGCAACTGTAGGAAGCAACA
GAGTTACCTTGTATGAATGTCATTCACAAGGAGAAATCCGGTTGTTGCAA
TCTTACGTGGATGCTGATGCTGATGAAAACTTTTACACTTGTGCATGGAC
CTATGATAGCAATACGAGCCATCCTCTGCTGGCTGTAGCTGGATCTAGAG
GCATAATTAGGATAATAAATCCTATAACAATGCAGTGTATAAAGCACTAT
GTTGGCCATGGAAATGCTATCAATGAGCTGAAATTCCATCCAAGAGATCC
AAATCTTCTCCTGTCAGTAAGTAAAGATCATGCTTTACGATTATGGAATA
TCCAGACGGACACTCTGGTGGCAATATTTGGAGGCGTAGAAGGGCACAGA
GATGAAGTTCTAAGTGCTGATTATGATCTTTTGGGTGAAAAAATAATGTC
CTGTGGTATGGATCATTCTCTTAAACTTTGGAGGATCAATTCAAAGAGAA
TGATGAATGCAATTAAGGAATCTTATGATTATAATCCAAATAAAACTAAC
AGGCCATTTATTTCTCAGAAAATCCATTTTCCTGATTTTTCTACCAGAGA
CATACATAGGAATTATGTTGATTGTGTGCGATGGTTAGGCGATTTGATAC
TTTCTAAGTCTTGTGAAAATGCCATTGTGTGCTGGAAACCTGGCAAGATG
GAAGATGATATAGATAAAATTAAACCCAGTGAATCTAATGTGACTATTCT
TGGGCGATTTGATTACAGCCAGTGTGACATTTGGTACATGAGGTTTTCTA
TGGATTTCTGGCAAAAGATGCTTGCATTGGGCAATCAAGTTGGCAAACTT
TATGTTTGGGATTTAGAAGTAGAAGATCCTCATAAAGCCAAATGTACAAC
ACTGACTCATCATAAATGTGGTGCTGCTATTCGACAAACCAGTTTTAGCA
GGGATAGCAGCATTCTTATAGCTGTTTGTGATGATGCCAGTATTTGGCGC
TGGGATCGACTTCGATAAAATACTTTTGCCTAATCAAAATTAAAATGTGT
TTGTTGTCTGTGTAAAATAAAATTAATGTATCTTGCTAGTAAGGGCACGT
AGAGCATTTAGAGTTGTCTTTCAGCATTCAATCAGGCTGAGCTGAATGTA
GTGATGTTTACATTGTTTACATTCTTTGTACTGTCTTCCTGCTCAGACTC
TACTGCTTTTAATAAAAATTTATTTTTG
YY1

SEQ ID NO:65

>gi|6042207|ref|NM_003403.2|Homo sapiens YY1
transcription factor (YY1), mRNA
AGGGCGAACGGGCGAGTGGCAGCGAGGCGGGGCGGGCTGAGGCCAGCGCG
GAAGTCTCGCGAGGCCGGGCCCGAGCAGAGTGTGGCGGCGGCGGCGAGAT
CTGGGCTCGGGTTGAGGAGTTGGTATTTGTGTGGAAGGAGGCGGAGGCGC
AGGAGGAGGAAGGGGGAAGCGGAGCGCGGCCCGGACGGGAGGAGGCGCGG
CCAGGGCGGGCGGTTGCGGCGAGGCGAGGCGAGGAGGGGGAGCCGAGACG
CAGCGGCCGAGGCGGGGGCGCGGGCGCACCGAGGCGAGGGAGGCGGGGAA
GCCCCGCCGCCGCCGCGGCGCCCGCCCCTTCCCCCGCCGCCCGCGCCCTC
TCCCCCCGCCGCGCTCGCCGCCTTCCTCCCTCGCTTCCTTCCCCACGGCC
GGCCGCCTCCTCGCCCGCCCGCCCGCAGCCGAGGAGCCGAGGCCGCCGGG
GCCGTGGCGGCGGAGCCCTCAGCCATGGCCTCGGGCGACACCCTCTACAT
CGCCACGGACGGCTCGGAGATGCCGGCCGAGATCGTGGAGCTGCACGAGA
TCGAGGTGGAGACCATCCCGGTGGAGACCATCGAGACCACAGTGGTGGGC
GAGGAGGAGGAGGAGGACGACGACGACGAGGACGGCGGCGGTGGCGACCA
CGGCGGCGGGGGCGGCCACGGGCACGCCGGCCACCACCACCACCACCATC
ACCACCACCACCACCCGCCCATGATCGCTCTGCAGCCGCTGGTCACCGAC
GACCCGACCCAGGTGCACCACCACCAGGAGGTGATCCTGGTGCAGACGCG
CGAGGAGGTGGTGGGCGGCGACGACTCGGACGGGCTGCGCGCCGAGGACG
GCTTCGAGGATCAGATTCTCATCCCGGTGCCCGCGCCGGCCGGCGGCGAC
GACGACTACATTGAACAAACGCTGGTCACCGTGGCGGCGGCCGGCAAGAG
CGGCGGCGGCGGCTCGTCGTCGTCGGGAGGCGGCCGCGTCAAGAAGGGCG
GCGGCAAGAAGAGCGGCAAGAAGAGTTACCTCAGCGGCGGGGCCGGCGCG
GCGGGCGGCGGCGGCGCCGACCCGGGCAACAAGAAGTGGGAGCAGAAGCA
GGTGCAGATCAAGACCCTGGAGGGCGAGTTCTCGGTCACCATGTGGTCCT
CAGATGAAAAAAAAGATATTGACCATGAGACAGTGGTTGAAGAACAGATC
ATTGGAGAGAACTCACCTCCTGATTATTCAGAATATATGACAGGAAAGAA
ACTTCCTCCTGGAGGAATACCTGGCATTGACCTCTCAGATCCCAAACAAC
TGGCAGAATTTGCTAGAATGAAGCCAAGAAAAATTAAAGAAGATGATGCT
CCAAGAACAATAGCTTGCCCTCATAAAGGCTGCACAAAGATGTTCAGGGA
TAACTCGGCCATGAGAAAACATCTGCACACCCACGGTCCCAGAGTCCACG
TCTGTGCAGAATGTGGCAAAGCTTTTGTTGAGAGTTCAAAACTAAAACGA
CACCAACTGGTTCATACTGGAGAGAAGCCCTTTCAGTGCACGTTCGAAGG
CTGTGGGAAACGCTTTTCACTGGACTTCAATTTGCGCACACATGTGCGAA
TCCATACCGGAGACAGGCCCTATGTGTGCCCCTTCGATGGTTGTAATAAG
AAGTTTGCTCAGTCAACTAACCTGAAATCTCACATCTTAACACATGCTAA
GGCCAAAAACAACCAGTGAAAAGAAGAGAGAAGACCCTTCTCGACCACGG
NAAGCATCTTCCAGAAGTGTGATTGGGAATAAATATGCCTCTCCTTTGTA
TATTATTTCTAGGAAGAATTTTAAAAATGAATCCTACACACCTAAGGGAC
ATGTTTTGATAAAGTAGTAAAAAAAAAAAAAAAAAAACTTTACTAAGATG
ACATTGCTAAGATGCTCTATCTTGCTCTGTAATCTCGTTTCAAAAACACA
GTGTTTTTGTAAAGTGTGGTCCCAACAGGAGGACAATTCATGAACTTCGC
ATCAAAAGACAATTCTTTATACAACAGTGCTAAAAATGGGACTTCTTTTC
ACATTCTTATAAATATGAAGCTCACCTGTTGCTTACAATTTTTTTAATTT
TGTATTTTCCAAGTGTGCATATTGTACACTTTTTTGGGGATATGCTTAGT
AATGCTACGTGTGATTTTTCTGGAGGTTGATAACTTTGCTTGCAGTAGAT
TTTCTTTAAAAGAATGGGCAGTTACATGCATACTTCAAAAGTATTTTCCT
GTAAAAAAAAAAAAAGTTATATAGGTTTTGTTTGCTATCTTAATTTTGGT
TGTATTCTTTGATGTTAACACATTTTGTATAATTGTATCGTATAGCTGTA
TTGAATCATGTAGTATCAAATATTAGATGTGATTTAATAGTGTTAATCAA
TTTAAACCCATTTTAGTCACTTTTTTTTTCCAAAAAAATACTGCCAGATG
CTGATGTTCAGTGTAATTTCTTTGCCTGTTCAGTTACAGAAAGTGGTGCT
CAGTTGTAGAATGTATTGTACCTTTTAACACCTGATGTGTACATCCCATG
TAACAGAAAGGGCAACAATAAAATAGCAATCCTAAAG
CBX1

SEQ ID NO:66

>gi|773207|emb|X77824.1|HSM3 H. sapiens chromobox
2 gene
CTGCCAGAGATGAGTGCAGGTGAGGAGAGTAGCAGCTCGGACTCCGACCC
CGACTCCGCCTCGCCGCCCAGCACTGGACAGAACCCATCAGTGTCCGTTC
AGACCAGCCAGGACTGGAAGCCCACCCGCAGCCTCATCGAGCACGTATTT
GTCACCGACGTCACTGCCAACCTCATCACCGTCACAGTGAAGGAGTCTCC
CACCAGCGTCGGCTTCTTCAACCTGAGGCATTACTGAAGCCCCGGCGCCA
CCAGCTGCGCGTCTTACTCCCCTTCCCTGCCTATGGTGTCGCTTGGCTAA
GTGACTCCCAGCCCAAGCCCCCTCAAGAGTCTGATCGTCGGGGGAGGAGG
AGTGGG
CBX6

SEQ ID NO:67

Attatgggctgtgggtgccgctgagcaagatggagctgtctgcagtgggc
gagcgggtctTcgcggccgaatccatcatcaaacggcggatccgaaaggg
acgcatcgagtacctggtgaAatggaaggggtgggcgatcaagtacagca
cttgggagcccgaggagaacatcctggactCgcggctcattgcagccttc
gaacaaaaggagagggagcgtgagctgtatgggcccaagaAgaggggacc
caaacccaaaactttcctcctgaaggcgcgggcccaggccgaggccctcc
Gcatcagtgatgtgcatttctctgtcaagccgagcgccagtgcctcctcg
cccaagctgcActccagcgcagccgtgcaccggctcaagaaggacatccg
ccgctgccaccgtatgtcccGccgtcccctgccccgcccggacccgcagg
ggggcagccccggactgcgcccgcccatttCgcccttctcggagacggtg
cgcatcatcaaccgcaaggtgaagccgcgggagcccaagcGgaaccgcat
catcctgaacctgaaggtgatcgacaagggcgctggcggcgggggcgccg
Ggcagggggccggggcgctggcccgccccaaagtcccctcgcggaaccgc
gttataggcaAgagcaagaagttcagcgagagcgtcctgcgtacacagat
ccgccacatgaagttcggcgCctttgcgctgtacaagcctccgcccgccc
ccctggtagccccgtcccccggcaaggctgAggcctcagccccgggccct
gggctacttctggccgcccccgccgccccctacgacgcccGcagctctgg
ctcctccggctgcccctcgcctacaccacagtcctctgaccccgacgaca
Cgccccccaagctcctccccgagaccgtgagcccatccgcccccagctgg
cgcgagccggAggtgctcgacctgtccctccctcccgagtcggcagccac
cagcaagcgggcaccgcctgAggtcacagctgctgccggcccggcacctc
ccacggcccctgagcccgccggtgcctcctCcgagcccgaggctggggac
tggcgccccgagatgtcaccctgctccaatgtggtcgtcaCcgatgtcac
cagcaacctcctgacggtcacaatcaaggaattctgcaaccctgaggatt
Tcgagaaggtggctgctggggtagcaggcgccgctgggggcggtggcagc
attggggcgaGcaagtga
HPC2 (CBX4)

SEQ ID NO:68

>gi|4503032|ref|NM_001880.1|Homo sapiens acti-
vating transcription factor 2 (ATF2), mRNA
GAATTCTGTGATAAGTTATTCAACTTATGAAATTCAAGTTACATGTGAAT
TCTGCCAGGCAATACAAGGACCTGTGGAATATGAGTGATGACAAACCCTT
TCTATGTACTGCGCCTGGATGTGGCCAGCGTTTTACCAACGAGGATCATT
TGGCTGTCCATAAACATAAACATGAGATGACACTGAAATTTGGTCCAGCA
CGTAATGACAGTCTCATTGTGGCTGATCAGACCCCAACACCAACAAGATT
CTTGAAAAACTGTGAAGAAGTGGGTTTGTTTAATGAGTTGGCGAGTCCAT
TTGAGAATGAATTCAAGAAAGCTTCAGAAGATGACATTAAAAAAATGCCT
CTAGATTTATCCCCTCTTGCAACACCTATCATAAGAAGCAAAATTGAGGA
GCCTTCTGTTGTAGAAACAACTCACCAGGATAGTCCTTTACCTCACCCAG
AGTCTACTACCAGTGATGAGAAGGAAGTACCATTGGCACAAACTGCACAG
CCCACATCAGCTATTGTTCGTCCAGCATCATTACAGGTTCCCAATGTGCT
GCTTACAAGTTCTGACTCAAGTGTAATTATTCAGCAGGCAGTACCTTCAC
CAACCTCAAGTACTGTAATCACCCAGGCACCATCCTCTAACAGGCCAATT
GTCCCTGTACCAGGCCCATTTCCTCTTCTGTTACATCTTCCTAGTGGACA
AACCATGCCTGTTGCTATTCCTGCATCAATTACAAGTTCTAATGTGCATG
TTCCAGCTGCAGTCCCACTCGTTCGACCAGTCACCATGGTGCCTAGTGTT
CCAGGAATCCCAGGTCCTTCCTCTCCCCAACCAGTACAGTCAGAAGCAAA
AATGAGATTAAAAGCTGCTTTGACCCAGCAACATCCTCCAGTTACCAATG
GTGATACTGTCAAAGGTCATGGTAGCGGATTGGTTAGGACTCAGTCAGAG
GAATCTCGACCGCAGTCATTACAACAGCCAGCCACATCCACTACAGAAAC
TCCGGCTTCTCCAGCTCACACAACTCCACAGACCCAAAGTACAAGTGGTC
GTCGGAGAAGAGCAGCTAACGAAGATCCTGATGAAAAAAGGAGAAAGTTT
TTAGAGCGAAATAGAGCAGCAGCTTCAAGATGCCGACAAAAAAGGAAAGT
CTGGGTTCAGTCTTTAGAGAAGAAAGCTGAAGACTTGAGTTCATTAAATG
GTCAGCTGCAGAGTGAAGTCACCCTGCTGAGAAATGAAGTGGCACAGCTG
AAACAGCTTCTTCTGGCTCATAAAGATTGCCCTGTAACCGCCATGCAGAA
GAAATCTGGCTATCATACTGCTGATAAAGATGATAGTTCAGAAGACATTT
CAGTGCCGAGTAGTCCACATACGGAAGCTATACAGCATAGTTCGGTCAGC
ACATCCAATGGAGTCAGTTCAACCTCCAAGGCAGAAGCTGTAGCCACTTC
AGTCCTCACCCAGATGGCGGACCAGAGTACAGAGCCTGCTCTTTCACAGA
TCGTTATGGCTCCTTCCTCCCAGTCACAGCCCTCAGGAAGTTGATTAAAA
ACCTGCAGTACAACAGTTTAGATACTCATTAGTGACTTCAAAGGGAAATC
AAGGAAAGACCAGTTTCCATTTATGCGAAATCTGTGGTTGTAAATTT
HPC3

SEQ ID NO:69

>gi|10190681|ref|NM_020649.1|Homo sapiens chromo-
box homolog 8 (Pc class homolog, Drosophila)
(CBX8), mRNA
CTGACGTCAGCGGGAGAGTATTATGGTCTGTCGTGCGCTGGCTGCTGCTT
TTCTGCTCCTGGAAGCGGCCTAGGGGGGAAGCGGCGAGTCAACATGGAGC
TTTCAGCGGTGGGGGAGCGGGTGTTCGCGGCCGAAGCCCTCCTGAAGCGG
CGCATACGGAAAGGACGCATGGAATACCTCGTGAAATGGAAGGGATGGTC
GCAGAAGTACAGCACATGGGAACCGGAGGAAAACATCCTGGATGCTCGCT
TGCTCGCAGCCTTTGAGGAAAGGGAAAGAGAGATGGAGCTCTATGGCCCC
AAAAAGCGTGGACCCAAGCCCAAAACCTTCCTCCTCAAAGCGCAGGCCAA
GGCAAAGGCCAAAACTTACGAGTTTCGAAGTGACTCAGCCAGGGGCATCC
GGATCCCCTACCCTGGCCGCTCGCCCCAGGACCTGGCCTCCACTTCCCGG
GCCCGGGAGGGCCTTCGAAACATGGGTTTGTCCCCGCCAGCGAGCAGCAC
CAGCACCAGCAGCACCTGCCGCGCAGAGGCCCCTCGGGACCGGGACCGAG
ACCGGGATAGGGACCGGGAGCGGGATCGAGAAAGGGAGAGGGAGCGAGAG
AGGGAGCGGGAACGTGAGAGGGAACGAGAGCGGGGTACCAGCAGAGTGGA
TGACAAGCCCAGCTCACCGGGGGACAGCTCGAAGAAGCGAGGCCCCAAGC
CCCGGAAGGAGCTCCCGGACCCCTCACAGAGGCCCTTAGGCGAACCCAGC
GCCGGCCTCGGAGAGTACCTCAAGGGCAGGAAGCTGGACGACACCCCTTC
CGGGGCAGGAAAGTTTCCAGCCGGCCACAGTGTGATCCAGCTGGCCCGAA
GACAGGACTCGGACCTGGTGCAGTGTGGTGTGACCAGCCCTAGCTCAGCT
GAGGCCACGGGCAAACTGGCTGTGGACACCTTCCCGGCCAGGGTGATAAA
GCACAGGGCTGCCTTCCTGGAGGCCAAAGGCCAGGGTGCCCTAGATCCCA
ATGGCACCCGGGTCCGACATGGCTCAGGCCCCCCCAGCTCTGGGGGGGGC
CTGTACCGGGACATGGGGGCCCAGGGGGGAAGGCCCTCCCTCATCGCCAG
GATCCCTGTGGCCAGAATCCTGGGGGACCCGGAGGAAGAGTCCTGGAGCC
CCTCCCTGACTAACCTGGAGAAGGTGGTGGTCACGGACGTGACCTCAAAC
TTTTTGACCGTCACCATTAAGGAAAGTAACACGGACCAAGGCTTTTTTAA
AGAGAAAAGATGAATGCTGGGTGGGTGTGTCCAGGAGGAGAGCAGGGGAG
AGAGTGAGCGTGAGCTTGGCATAGTGATTTTTATTTCTGGGTGGGATGTG
GCCTTTTGGCTGGTCCCGTCCCTGATGTCACCCCCACCCCACCAGCCCCT
TTCATCCCTCCTTCCTCCCCCTCAGTTTTTGTTGGAAAGATTATCTCTAG
AGTTATATTTTCTATTAGATGTAAATATGTTATTTAAGAAAAATATCTAA
ATATATATATTTCAACTCGAAAAAAAAAAAAAAAAACTCGAG
PHF1

SEQ ID NO:70

>gi|13435396|ref|NM_024165.1|Homo sapiens PHD
finger protein 1 (PHF1), transcript variant 2,
mRNA
CTCCTCCTGCCGCTGCCGCTGCTTTGGCTGCTGCGTCATACGCCCCAGAG
CCGCCGGGACGGAGGGGCTGGGCCTGGGGACCCCCCGGCCTCCGCCTGCA
CGCCCCCCCACGCCCGGACGTGCCCTCTCCGCGCGGGGGACTCGCCTAGG
TCTCCTACGTCTGCCCCTGCCCGGCTCCCGGCGGCCCCAGCTGTCACCGG
CCCCCCCAGGATGCAATGGCGCAGCCCCCCCGGCTGAGCCGCTCTGGTGC
CTCCTCACTTTGGGACCCAGCTTCTCCTGCTCCCACCTCTGGCCCCAGGC
CTCGGCTTTGGGAGGGTCAAGATGTGCTGGCCAGATGGACTGATGGGCTG
CTATACTTGGGTACCATCAAAAAGGTGGACAGTGCTAGGGAGGTGTGTCT
GGTCCAGTTTGAGGATGATTCGCAGTTTCTGGTTCTATGGAAAGACATTA
GCCCTGCTGCCCTCCCTGGAGAGGAACTCCTCTGTTGTGTCTGTCGCTCT
GAGACTGTGGTCCCTGGGAACCGGCTGGTCAGCTGTGAGAAGTGTCGCCA
TGCTTATCACCAGGACTGCCATGTTCCCAGGGCTCCAGCCCCTGGAGAGG
GAGAGGGCACATCCTGGGTATGCCGCCAGTGTGTCTTTGCGATCGCCACC
AAGAGGGGAGGTGCCCTGAAGAAGGGCCCCTATGCCCGGGCCATGCTGGG
TATGAAGCTTTCTCTGCCATATGGACTGAAGGGGCTGGACTGGGATGCTG
GACATCTGAGCAACCGACAGCAGAGTTACTGTTACTGTGGTGGCCCTGGG
GAGTGGAACCTGAAAATGCTGCAGTGCCGGAGCTGCCTGCAGTGGTTCCA
TGAGGCCTGCACCCAGTGTCTGAGCAAGCCCCTCCTCTATGGGGACAGGT
TCTATGAATTTGAATGCTGTGTGTGTCGCGGGGGCCCTGAGAAAGTCCGG
AGACTACAGCTTCGCTGGGTGGATGTGGCCCATCTTGTCCTGTATCACCT
CAGTGTTTGCTGTAAGAAGAAATACTTTGATTTTGATCGTGAGATCCTCC
CCTTCACTTCTGAGAATTGGGACAGTTTGCTCCTGGGGGAGCTTTCAGAC
ACCCCCAAAGGAGAACGTTCTTCCAAGCTCCTCTCTGCTCTTAACAGCCA
CAAGGACCGTTTCATTTCAGGGAGAGAGATTAAGAAGAGGAAATGTTTGT
TTGGTCTCCATGCTCGGATGCCTCCCCCTGTGGAGCCCCCTACTGGAGAT
GGAGCACTCACCAGCTTCCCTTCAGGGCAGGGCCCTGGGGGAGGGGTCTC
ACGTCCCCTGGGGAAGCGCCGGAGGCCGGAGCCAGAGCCCCTGAGGAGGA
GGCAGAAGGGGAAAGTGGAGGAGCTGGGGCCACCCTCAGCAGTGCGCAAT
CAGCCCGAGCCCCAGGAGCAGAGGGAGCGGGCTCATCTGCAGAGGGCACT
GCAGGCCTCAGTGTCTCCACCATCCCCCAGCCCTAACCAGAGTTACCAGG
GCAGCAGCGGCTACAACTTCCGGCCCACAGATGCCCGCTGCCTGCCCAGC
AGCCCCATCCGGATGTTTGCTTCCTTCCACCCTTCTGCCAGCACCGCAGG
GACCTCTGGGGACAGTGGACCCCCAGACAGGTCACCCCTGGAACTTCACA
TTGGTTTCCCCACAGACATCCCTAAAAGTGCCCCCCACTCGATGACTGCC
TCATCTTCCTCAGTTTCATCCCCATCCCCAGGTCTTCCTAGACGCTCAGC
ACCCCCTTCTCCCCTGTGCCGTAGTTTGTCTCCTGGGACTGGGGGAGGAG
TCCGAGGTGGGGTTGGTTACCTGTCCCGAGGGGACCCTGTCCGGGTCCTT
GCTCGGAGAGTACGGCCTGATGGCTCTGTGCAGTACCTGGTTGAGTGGGG
AGGAGGGGGCATCTTCTGAACAGCCTGCCTCTGCCCAGCTCCCCATTCAC
ACACACCGGCACTTTCATACCCTGACCTCTGACCTCACCTACAGCTGGGA
TGTACCTGGAGAGATAGGGGGTAGTTCTCCCTACTGCCCAGGCTGGAATC
CAAGAGTGGGGAGTGGGGAAGAGGCCCTCTTCTCTACCCTCCTTCATGAT
TCCTGACCCCTCCCATCCTTCCCATTTCCTTTGATGTTATTTTGTTACAG
CTTTTTAAATATTTTTTAAAATTATTTAACCCCTGGGGGCAGAGACTGAG
GAGGGAGGATGATAAGGGATCCCGGACTCTGTATGATTGAAATAAAGAGA
AATAAACAAA
PHF2

SEQ ID NO:71

>gi|4885546|ref|NM_005392.1|Homo sapiens PHD
finger protein 2 (PHF2), mRNA
GCGGCCGCTCGGCGGCCGGGGGTCCCTTCGGTGGGGCCGCGGCTCCCCGC
CCGCCGCCCCCGCGCGTCCATTCGCTTTGTGTCCCGCGCGCGGCCGGGCC
CCCCGCGCACTCTCAGCCCTGCGCCCCGCGGCCCGGCGGGCGGCTCCCGG
CGCGGCCCCAGCAGCCCGCGCCGGCATTGTGTGGACGCGCCCGGCCGCGA
GCGCGCGCGCGGGCCCTGCCGAGCGCCCCCGGCCCCGTCCGCTCCGGCCG
CGGCGCCCGCGCCCGCCGCCCCCGCCGCCCTCGCCGCGCGGCCCCCGGCC
CGGCCCGGCCCGACCCGGGCAGCGCAGCGGCGGGGCGAGCGGCGGCGCGG
CAACATGGCGACGGTGCCCGTGTACTGCGTCTGCCGGCTGCCCTACGACG
TTACCCGCTTTATGATCGAGTGCGATGCCTGCAAGGACTGGTTCCACGGC
AGCTGTGTTGGGGTGGAAGAGGAAGAGGCACCAGACATCGACATTTACCA
CTGCCCGAACTGCGAGAAAACCCATGGCAAGTCCACACTCAAGAAAAAGC
GGACTTGGCACAAACACGGCCCTGGGCCAACACCGGACGTGAAACCAGTG
CAGAATGGCAGTCAGCTGTTCATCAAGGAGCTGCGGAGCCGAACCTTCCC
CAGTGCTGAAGACGTGGTGTCCCGTGTGCCAGGTAGCCAGCTCACCGTGG
GCTACATGGAGGAGCATGGCTTCACTGAGCCCATCCTTGTCCCCAAGAAA
GATGGCCTGGGCTTAGCTGTCCCTGCCCCAACATTCTACGTGAGTGACGT
CGAGAACTACGTGGGGCCGGAACGGAGTGTGGATGTGACAGATGTCACCA
AGCAGAAGGACTGCAAGATGAAGCTGAAGGAGTTTGTGGACTATTACTAC
AGCACCAACCGCAAGCGGGTCCTCAACGTCACCAACCTCGAGTTCTCTGA
CACCCGAATGTCCAGCTTCGTGGAGCCACCTGACATTGTAAAGAAACTGT
CATGGGTAGAAAACTACTGGCCAGATGATGCATTGCTGGCCAAGCCCAAA
GTGACCAAGTACTGCCTAATCTGCGTGAAGGACAGTTACACCGACTTCCA
CATCGACTCTGGGGGCGCCTCTGCCTGGTACCACGTGCTCAAGGGGGAGA
AGACCTTCTATCTCATCAGGCCGGCCTCGGCCAACATCTCCCTGTATGAG
CGCTGGCGGTCTGCCTCTAACCACAGCGAGATGTTCTTTGCTGACCAGGT
CGACAAATGCTACAAGTGCATCGTCAAGCAGGGCCAGACCCTCTTCATCC
CCTCAGGCTGGATCTACGCCACACTCACCCCTGTGGACTGCCTGGCCTTC
GCGGGACATTTCCTCCACAGCCTGAGTGTGGAGATGCAGATGAGAGCATA
CGAGGTGGAAAGGAGGTTGAAACTTGGCAGCCTGACTCAGTTTCCCAACT
TTGAAACTGCGTGCTGGTACATGGGAAAGCACCTATTGGAGGCGTTCAAA
GGTTCTCACAAGTCTGGGAAGCAGCTGCCCCCACATCTAGTCCAAGGAGC
TAAAATTCTCAATGGTGCTTTCCGATCGTGGACGAAGAAGCAGGCTTTGG
CAGAGCATGAGGACGAGCTCCCGGAGCACTTCAAACCTTCACAGCTAATC
AAGGACCTGGCCAAAGAGATCCGGCTCAGTGAGAATGCCTCCAAAGCCGT
CCGACCGGAAGTGAATACTGTCGCCTCGTCAGATGAGGTGTGTGACGGGG
ACCGGGAGAAGGAGGAGCCCCCGTCTCCCATTGAGGCCACCCCGCCTCAA
TCCCTCCTGGAGAAAGTGTCCAAAAAAAAGACTCCCAAAACTGTGAAGAT
GCCCAAGCCATCCAAAATCCCCAAGCCCCCGAAGCCCCCTAAGCCCCCAA
GGCCCCCCAAAACGCTGAAGCTCAAAGATGGAGGCAAGAAGAAAGGGAAG
AAGTCCCGGGAGTCAGCCTCACCCACCATCCCCAACCTGGACCTGCTCGA
AGCCCACACCAAGGAGGCACTGACCAAGATGGAGCCGCCCAAGAAGGGCA
AGGCCACAAAGAGTGTCCTGAGTGTGCCCAACAAAGATGTGGTTCACATG
CAGAATGATGTGGAGAGGCTGGAAATTCGAGAGCAAACAAAGAGCAAGTC
AGAAGCCAAGTGGAAATACAAGAACAGCAAACCTGACTCGTTACTGAAGA
TGGAGGAGGAGCAGAGGCTGGAGAAGTCGCCCCTGGCTGGGAACAAGGAC
AAGTTTTCCTTTTCTTTCTCCAACAGAAAACTCCTGGGCTCCAAGGCCCT
CAGGCCCCCGAGCAGCCCTGGTGTGTTCGGCGCCTTGCAGAGCTTCAAGG
AGGACAAGGCCAAGCCCGTGCGCGATGAGTATGAGTACGTATCAGATGAT
GGGGAGCTGAAGATAGACGAGTTTCCCATCAGGAGGAAGAAGAGCGCCCC
CAAAAGGGACTTGTCCTTCTTGTTAGACAAGAAGGAGGCTCTCCTCATGC
CCACCTCGAAGCCAAAGCTGGATTCTGCGGTGTACAAGAGCGATGACTCC
TCTGACGAGGGCTCTCTGCACATCGACACGGACACCAAGCCAGGCAGAAA
TGCCAAAGTGAAGAAGGAGAGTGGGAGCTCCGCGGCCGGCATCCTGGACC
TGCTGCAGGCCAGCGAGGAGGTTGGCGCACTCGAGTACAACCCCAACAGC
CAGCCCCCTGCCTCCCCCAGCACACAGGAAGCCATTCAGGGAATGCTCTC
CATGGCCAATCTGCAGGCCTCTGACTCTTGCCTGCAGACCACATGGGGCA
CGGGGCAGGCCAAGGGTGGCTCACTGGCAGCCCATGGTGCCCGGAAGATT
GGTGGTGGCAACAAAGGCACAGGCAAGCGCCTGCTGAAGAGGACTGCCAA
GAACAGTGTGGATCTGGAGGACTACGAGGAGCAGGATCACCTGGATGCCT
GCTTCAAGGACTCAGACTATGTTTACCCCTCACTGGAGTCTGACGAAGAT
AACCCCGTCTTCAAGTCCCGGTCAAAGAAGAGGAAAGGCTCAGACGATGC
TCCGTACAGCCCCACAGCCAGGGTCGGTCCATCGGTGCCAAGACAAGACA
GGCCTGTGCGTGAGGGGACCAGAGTGGCCTCCATTGAGACGGGGCTGGCA
GCTGCTGCAGCCAAGCTGTCCCAGCAGGAGGAGCAGAAAAACAGGAAGAA
GAAGAACACCAAAAGGAAGCCGGCTCCTAACACTGCCTCCCCCTCCATCT
CCACCTCTGCCTCCGCCTCCACGGGTACCACCTCGGCCTCCACCACCCCA
GCATCCACCACCCCGGCCTCCACCACCCCAGCATCCACCACCCCGGCCTC
CACCAGCACAGCCAGCAGCCAGGCCTCACAGGAGGGCAGCTCACCTGAGC
CCCCACCTGAATCACACAGCAGTAGCCTGGCTGACCACGAATATACAGCA
GCCGGCACATTCTCGGGGTCCCAGGCTGGCCGTGCCTCCCAGCCCATGGC
CCCTGGAGTCTTTCTCACACAGAGGCGGCCTTCTGCATCATCCCCCAACA
ACACTGCTGCCAAAGGAAAACGTACAAAAAAGGGCATGGCCACCGCCAAG
CAAAGGCTTGGAAAGATCTTGAAGATCCATCGGAATGGGAAACTGCTCCT
CTAAGGCTTGGAAAGCCAGGATCCTTCTGATATGCTAAGGACCCCCGGAG
CCCCGCTACATCAGCCCCTCCCAGGACGGTGGCTGTGCCGCCTGGCCCGG
GGAGGGCTTGCTTCATTCCGACCAATTTTCCAATCAA
HPH1

SEQ ID NO:72

>gi|11038623|ref|NM_004426.1|Homo sapiens early
development regulator 1 (polyhomeotic 1 homolog)
(EDR1), mRNA
CCCGCCCTCGGCGCCCCCGCCCCTCCAGAAAGGGGAGGAGGCGAGGGGAG
CCCGCCGCGGAGGCCGAGCGAGCCGGCGCCCAGCCCAGCCTGGCGACTGG
GGACCCCGGCACATGAGGTGGACGCCCCGGGGAAGACTTGGGTGCACAGC
CAGGCGAGAAGGTCTTGAGTCAGACAGAGCACCAGCCTTGGGGACCCTGG
ACCACTATCATGGAGACTGAGAGCGAGCAGAACTCCAATTCCACCAATGG
GAGTTCTAGCTCAGGGGGCAGCTCTCGGCCCCAGATAGCTCAAATGTCAC
TTTATGAACGACAAGCAGTGCAGGCTCTGCAAGCACTGCAGCGGCAGCCC
AATGCAGCTCAGTATTTCCACCAGTTCATGCTCCAGCAGCAGCTCAGTAA
TGCCCAGCTGCATAGCCTGGCTGCCGTCCAGCAGGCCACAATTGCTGCCA
GTCGGCAGGCCAGCTCCCCAAACACCAGCACTACACAGCAGCAGACTACC
ACCACCCAGGCCTCGATCAATCTGGCCACCACATCGGCCGCCCAGCTCAT
CAGCCGATCCCAGAGTGTGAGCTCTCCCAGTGCTACCACCTTGACCCAAT
CTGTGCTACTGGGGAACACCACCTCCCCACCCCTCAACCAGTCTCAGGCC
CAGATGTATCTACGGCCACAGCTGGGAAACCTATTGCAGGTAAACCGAAC
CCCTGGGTCGAATGTGCCTCTAGCCTCCCAACTCATCCTGATGCCTAATG
GGGCGGTGGCTGCAGTCCAGCAGGAGGTGCCATCTGCTCAGTCTCCTGGA
GTTCATGCAGATGCAGATCAGGTTCAGAACTTGGCAGTAAGGAATCAACA
GGCCTCAGCTCAAGGACCTCAGATGCAAGGCTCCACTCAGAAGGCCATTC
CTCCAGGAGCCTCCCCTGTCTCTAGCCTCTCCCAGGCCTCTAGCCAGGCC
CTAGCGGTGGCACAGGCTTCCTCTGGGGCCACAAACCAGTCCCTCAACCT
TAGTCAAGCTGGTGGAGGCAGTGGGAATAGCATCCCAGGGTCCATGGGTC
CAGGTGGAGGTGGGCAGGCACATGGTGGTTTGGGTCAGTTGCCTTCCTCA
GGAATGGGTGGTGGGAGCTGTCCCAGGAAGGGTACAGGAGTGGTGCAGCC
CTTGCCTGCAGCCCAAACAGTAACTGTGAGCCAGGGCAGCCAGACAGAGG
CAGAAAGTGCAGCAGCCAAGAAGGCAGAAGCAGATGGGAGTGGCCAGCAG
AATGTGGGCATGAACCTGACACGGACAGCCACACCTGCGCCCAGCCAGAC
ACTTATTAGCTCAGCCACCTACACACAGATCCAGCCCCATTCACTGATTC
AGCAACAGCAACAGATCCACCTCCAGCAGAAACAGGTGGTGATCCAGCAG
CAGATTGCCATCCACCACCAGCAGCAGTTCCAGCACCGGCAGTCCCAGCT
CCTTCACACAGCTACACACCTCCAGTTGGCGCAGCAGCAGCAGCAGCAAC
AACAGCAACAGCAGCAACAGCAGCAGCCGCAAGCCACCACCCTCACTGCC
CCTCAGCCACCACAGGTCCCACCTACTCAGCAGGTCCCACCTTCCCAGTC
CCAGCAGCAAGCCCAAACCCTGGTCGTTCAGCCCATGCTTCAGTCTTCAC
CCTTGTCTCTTCCACCTGATGCAGCCCCTAAGCCACCAATTCCCATCCAA
TCCAAACCACCTGTAGCACCTATCAAGCCGCCTCAGTTAGGGGCCGCTAA
GATGTCAGCTGCCCAGCAACCACCACCCCATATCCCTGTGCAAGTTGTAG
GCACTCGACAGCCAGGTACAGCCCAGGCACAGGCTTTGGGGTTGGCACAG
CTGGCAGCTGCTGTACCTACTTCCCGGGGGATGCCAGGTACAGTGCAGTC
TGGTCAGGCCCTAAAGGCTTCCTCGCCACCTTCATCCCAGGCTCCTGGTG
CACTGCAGGAGTGCCCTCCCACATTGGCCCCGGGGATGACCCTTGCTCCT
GTGCAGGGGACAGCACATGTGGTAAAGGGTGGGGCTACCACCTCCACACC
TGTTGTAGCCCAGGTCCCTGCTGCCTTCTATATGCAGTCTGTGCACTTGC
CGGGAAAACCCCAGACATTGGCTGTCAAACGCAAGGCTGACTCTGAGGAG
GAGAGAGATGATGTCTCCACATTGGGTTCAATGCTTCCTGCCAAGGCATC
TCCAGTAGCAGAAAGCCCAAAAGTCATGGACGAGAAGAGCAGTCTTGGAG
AAAAAGCTGAATCAGTGGCTAATGTGAATGCTAATACTCCAAGCAGTGAA
CTAGTAGCCTTGACCCCCGCCCCTTCAGTACCGCCTCCTACACTAGCCAT
GGTGTCTAGACAAATGGGTGACTCAAAACCCCCACAGGCCATCGTGAAGC
CCCAGATTCTCACCCACATCATTGAAGGCTTTGTTATCCAGGAAGGAGCA
GAACCTTTCCCCGGTGGTTGTTCTCAGTTACTGAAGGAGTCTGAGAAGCC
ACTACAGACTGGCCTTCCGACAGGCTTGACTGAGAATCAGTCAGGTGGCC
CCTTTGGAGTGGACAGCCCATCTGCTGAGTTAGATAAGAAGGCGAATCTC
CTGAAGTGCGAGTACTGTGGGAAGTACGCCCCCGCAGAGCAGTTTCGTGG
CTCTAAGAGGTTCTGCTCCATGACCTGCGCTAAGAGGTACAATGTGAGCT
GTAGCCATCAGTTCCGGCTGAAGAGGAAAAAAATGAAAGAGTTTCAAGAA
GCCAACTATGCTCGCGTTCGCAGGCGTGGACCCCGCCGCAGCTCCTCTGA
CATTGCCCGTGCCAAGATTCAGGGCAAGTGCCACCGGGGTCAAGAAGACT
CTAGCCGGGGTTCAGATAATTCCAGTTATGATGAAGCACTCTCTCCAACA
TCTCCTGGGCCTTTATCAGTAAGAGCTGGGCATGGAGAACGTGACCTGGG
GAATCCCAATACAGCTCCACCTACACCGGAATTACATGGCATCAACCCTG
TGTTCCTGTCCAGTAATCCCAGCCGTTGGAGTGTAGAGGAGGTGTACGAG
TTTATTGCTTCTCTCCAAGGCTGCCAAGAGATTGCAGAGGAATTTCGCTC
ACAGGAGATTGATGGACAGGCCTTTTTATTACTTAAAGAAGAACATCTTA
TGAGTGCCATGAACATCAAGCTGGGCCCTGCCCTCAAGATCTGTGCCAAG
ATCAATGTCCTCAAGGAGACCTAAGGTGGCCCTCTTGCACAAACCAGCCT
AAGGCAGACACTCTCCACTGTCCAGGTTATAACCTGGTACCAGCAGACTT
TGCAGGGAAGAAAGAGTTGTTCCAATCATGTAACCTTCTGTAGGGGATTA
CTGAGACAGGGAAGAGAAGTGCAAGAATTGGTTGCTGGTGCTACATGGCG
GCAGCTTTGACATTTTCTCTGGGTTCTACTTTATTTTTTAAAATCTTTAC
AGTTCTCACCATTTCACGTACCTTAATCCAATCTTTATAAAAGAGGCAGT
CTAGAGAACTAGGACTGCTCAGCCTTATCCTGGAGTGGAGCATTTAGCCC
AGGTCTTAATTCTCCAAGAGGAGGAATACATAGTATGGTAAGGCAAGGAA
CTGGGTGGAATGTCAGGTTGCCTGCCCAATGGGAGAGGTAGGGTTTTTCT
AGCTTGTGTGACAGAAGTAGCAAAATCTGGTCCTCCCCCCTCCCAGTGTA
GCTGTGGCTCAGAGTTTTTTCTTTTTGTTGTCACTTACTCCCTTGTGATT
GAATTTTTTCTCCTGCATCCATGGCAGGATCCCCAGCCAGTATAGAGACT
TGGTTGGCATCTTCTGCTGCAGGGACTAAAAGTATTTGACTGGGGCACAT
GTGGCTGTTGTCATTCTTTCTGCATCCCACTGTTCCCCTCCAATTTATGT
TATTTTCTACCCTGTTTTTCAGTTCCATCTCTGCTCTGTCCTATAGCTTT
ATAAAACCAGAGTGTGTGGGGCTGAGGTCAGGAGTATAAGTACCTGCCTT
AGGCACTATTCCTTATATAACAAAAATATTAAATATTTTTTTCCTCAGTA
AAAGGATG
HPH2

SEQ ID NO:73

>gi|4758241|ref|NM_004427.1|Homo sapiens early
development regulator 2 (polyhomeotic 2 homolog)
(EDR2), mRNA
GGCGCCGCATGTGTCTCCGCGGCGGCTGCAGCCCTCGAGCGCCCGCCGCC
GCGCCCCAACCCCGGCCGCCGCCCGCCCTCCCGCCCCGGCCTCGCGCCCC
CGTCCCGGCCTCGCGCCCCGGCCGCCCTTTGTTGACGCCGGCCAGGCCGT
GCGGTCGGATGCGCCGCGGCAGCCCCGGGCCCCGGCTCGGAGGCTCCCGG
GGCGAGAGGAGGCGGCCCGCCGGCCGGGACCCCGCGCGAGTCGGCCCCGG
CCAGGGGCTGCGTAGGCCCGCCCGGCCAGGCCCAGCCGCCTGGACAGAGA
CAGGGCAGGGCATTGTTCATGCACTGACCGACCTCAGCATCCCCGGCATG
ACCTCAGGGAACGGAAACTCTGCCTCCAGCATCGCCGGCACTGCCCCCCA
GAATGGTGAGAATAAACCACCACAGGCCATTGTGAAACCCCAAATCCTGA
CGCATGTTATCGAAGGGTTTGTGATCCAGGAGGGGGCGGACGTTTCCCGG
TGGGACGCTCGTCTGCTGGTGGGGAATCTCAAGAAGAAGTATGCACAGGG
GTTCCTGCCTGAGAAACTTCCACAGCAGGATCACACCACCACCACTGACT
CGGAGATGGAGGAGCCCTATCTGCAAGAATCCAAAGAGGAGGGTGCTCCC
CTCAAACTCAAGTGTGAGCTCTGTGGCCGGGTGGACTTTGCCTATAAGTT
CAAGCGTTCCAAGCGCTTCTGTTCCATGGCTTGTGCAAAGAGGTACAACG
TGGGATGCACCAAACGGGTGGGACTTTTCCACTCAGACCGGAGCAAGCTG
CAGAAGGCAGGAGCTGCGACCCACAACCGCCGTCGGCCAGCAAAGCCAGT
CTGCCACCACTTACCAAGGATACCAAGAAGCAGCCAACAGGCACTGTGCC
CCTTTCGGTTACTGCTGCTTTGCGTAACACACAGCCAGGAAGACTCCAGC
CGTTGCTCAGATAACTCAAGCTATGAGGAACCCTTGTCACCCATCTCAGC
CAGCTCATCTACTTCCGCCGGCGACAAGGCCAGCGGGACCTGGAGCTCCC
CGACATGCATATGCGGGACCTGGTGGGCATGGGACACCACTTCCTGCCAA
GTGAGCCACCAAGTGAATGTAGAAGACGTCTACGAATTCATCCGCTCTCT
GCCAGGCTGCCAGGAGATAGCAGAGGAATTCCGTGCCCAGGAAATCGACG
GGCAAGCCCTGCTGCTGCTCAAGGAGGACCACCTGATGAGCGTTATGAAC
ATCAAGCTGGGGCCCGCCCTGAAGATCTACGCCCGCATCAGCATGCTCAA
GGACTCCTAGGGCTGGTGGCACCAGGATTCTGGCCCAGGGCGCCTCCTCC
CGACTGAGCAGAGCCAGACAGACATTCCTGAGGGGCCCAGAAATGGCGGC
GTTGGAGGGCAGGGGCTCTCCCTAGGGGCATAGCTGGTGAGGAGGTCTGG
GCACCTCCTCCATGGCTCTCAGGGGCCTTTCATTTCTGTGGGAGGGGCAG
AGAGGTAGGTGGCACAGAAGATGGGGCTTTATGCTTGTAAATATTGATAG
CACTGGCTTCCTCCAAAGTCCCAATACTCTAGCCCCGCTCTCTTCCCCTC
TTTCTGTCCCCCATTTTCCAGGGGGTATATGGTCAGGGCTCCCCAACCTG
AGTTGGTTACTTCAAGGGCAGCCAGCAGGCCTGGATGGAGGCCTAGAAAG
CCCTTGCCTTCCTTCCTCCCACTTCTTTCTCCAGGCCTGGTTAACTCTTC
CGTTGTCAGCTTCTCCCCCTTCAGCCTGTTTCTGCAGCAGCCAGGGTTCT
CCCCCCTACACCCTCTGCAGGTGGAGAGAGAGAAGCTGGGCCCAGCCGCG
GTGCCTGCTGGCCAAGACGCCTTAACGCTGTGTGTATGACTGTGTGACTG
TGTGGGAGCCTGGACTGACAGATAGGCCAAGGGCTACTCTCTGGCATCTC
CAGGTGTTTTGTAGCAAACAGCCACTTAGTGCTTTGTCCTGGACTCCACT
CAGCCTCAGGATGGGGAATAGCCAAGAATGGCAGCCTCAGCGCAGAGGCA
AGGTCAGAAAGAGACGGCGCTTCAGAGTTTCCTTTCCAGACACCCCTCCC
CGCACTGTGAAGTTCCCCTGACCGCCCTCCTGGTTCACAAAGAGCATTAA
GAAAGCTGCGGTGGTCTGAGCAACATAGCCCAGACGTGGAGCCTCCTGGC
CTGCCTGCCCGCCCACCCTGGGAGTCCAGTGGTGAGGCTCAGAGAACTTC
TAAGGGGAAAGAACAGCTGGAGTTTCTGTTGATGTGAAGAAGGCAGCTCT
TGGCCTCCCACTCCCACACTTCTTTGCCTATAAATCTTCCTAGCAGCAAT
TTGAGCTACCTGAGGAGGAGGCAGGGCAGAAGGGCAAGGGCCTGCCTCTG
ACCTGCCGTGTCCTTTGCAGGAAGGAGGTAGGCACCTTTCTGAGCTTATT
CTATTCCCCACCCACACCCCCAGGCAGGGTTGGAAATGAAGGACTTTTTT
AACCTTTGTTTTGTTTTTTAAAAATAAATCTGTAAAATCTGAAAAAAAAA
AAAAA
SSX1

SEQ ID NO:74

>gi|5032120|ref|NM_005635.1|Homo sapiens synovial
sarcoma, X breakpoint 1 (SSX1), mRNA
CACTTTGTCACCAACTGCTGCCAACTCGCCACCACTGCTGCCGCAATCGC
AACCACTGCTTTGTCTCTGAAGTGAGACTGCTCCTGGTGCCATGAACGGA
GACGACACCTTTGCAAAGAGACCCAGGGATGATGCTAAAGCATCAGAGAA
GAGAAGCAAGGCCTTTGATGATATTGCCACATACTTCTCTAAGAAAGAGT
GGAAAAAGATGAAATACTCGGAGAAAATCAGCTATGTGTATATGAAGAGA
AACTATAAGGCCATGACTAAACTAGGTTTCAAAGTCACCCTCCCACCTTT
CATGTGTAATAAACAGGCCACAGACTTCCAGGGGAATGATTTTGATAATG
ACCATAACCGCAGGATTCAGGTTGAACATCCTCAGATGACTTTCGGCAGG
CTCCACAGAATCATCCCGAAGATCATGCCCAAGAAGCCAGCAGAGGACGA
AAATGATTCGAAGGGAGTGTCAGAAGCATCTGGCCCACAAAACGATGGGA
AACAACTGCACCCCCCAGGAAAAGCAAATATTTCTGAGAAGATTAATAAG
AGATCTGGACCCAAAAGGGGGAAACATGCCTGGACCCACAGACTGCGTGA
GAGAAAGCAGCTGGTGATTTATGAAGAGATCAGTGACCCTGAGGAAGATG
ACGAGTAACTCCCCTGGGGGATACGACACATGCCCTTGATGAGAAGCAGA
ACGTGGTGACCTTTCACGAACATGGGCATGGCTGCGGCTCCCTCGTCATC
AGGTGCATAGCAAGTG
SSX2

SEQ ID NO:75

>gi|10337582|ref|NM_003147.1|Homo sapiens synovial
sarcoma, X breakpoint 2 (SSX2), mRNA
CTCTCTTTCGATTCTTCCATACTCAGAGTACGCACGGTCTGATTTTCTCT
TTGGATTCTTCCAAAATCAGAGTCAGACTGCTCCCGGTGCCATGAACGGA
GACGACGCCTTTGCAAGGAGACCCACGGTTGGTGCTCAAATACCAGAGAA
GATCCAAAAGGCCTTCGATGATATTGCCAAATACTTCTCTAAGGAAGAGT
GGGAAAAGATGAAAGCCTCGGAGAAAATCTTCTATGTGTATATGAAGAGA
AAGTATGAGGCTATGACTAAACTAGGTTTCAAGGCCACCCTCCCACCTTT
CATGTGTAATAAACGGGCCGAAGACTTCCAGGGGAATGATTTGGATAATG
ACCCTAACCGTGGGAATCAGGTTGAACGTCCTCAGATGACTTTCGGCAGG
CTCCAGGGAATCTCCCCGAAGATCATGCCCAAGAAGCCAGCAGAGGAAGG
AAATGATTCGGAGGAAGTGCCAGAAGCATCTGGCCCACAAAATGATGGGA
AAGAGCTGTGCCCCCCGGGAAAACCAACTACCTCTGAGAAGATTCACGAG
AGATCTGGACCCAAAAGGGGGGAACATGCCTGGACCCACAGACTGCGTGA
GAGAAAACAGCTGGTGATTTATGAAGAGATCAGCGACCCTGAGGAAGATG
ACGAGTAACTCCCCTCAGGGATACGACACATGCCCATGATGAGAAGCAGA
ACGTGGTGACCTTTCACGAACATGGGCATGGCTGCGGACCCCTCGTCATC
AGGTGCATAGCAAGTG

TABLE 3
Enhancers of trithorax and polycomb.

EPC1

SEQ ID NO:76

>gi|13376809|ref|NM_025209.1|Homo sapiens enhancer
of polycomb1 (EPC1), mRNA
ATGAGTAAACTGTCGTTTCGGGCGCGGGCGCTAGACGCCTCGAAGCGGCT
GCCGGTTTTCCGCTGTGAGGATCTGCCCCACCTGCACGAATACGCCTCGA
TAAACAGGGCCGTGCCGCAGATGCCCACCGGAATGGAGAAGGAAGAGGAG
TCGGAACATCATCTTCAGCGGGCTATTTCAGCACAGCAGGTGTATGGCGA
GAAGAGGGATAATATGGTTATACCGGTCCCAGAGGCAGAAAGTAATATTG
CTTACTATGAGTCTATATATCCTGGGGAATTTAAGATGCCAAAGCAGCTC
ATTCACATACAGCCTTTTAGTTTGGATGCTGAACAGCCTGATTATGATTT
GGATTCTGAAGATGAAGTATTTGTGAATAAACTGAAAAAGAAAATGGACA
TCTGCCCATTGCAATTTGAGGAGATGATTGACCGCCTAGAAAAAGGCAGT
GGTCAGCAGCCAGTCAGTCTGCAGGAAGCCAAACTACTGCTAAAAGAAGA
TGATGAACTAATTAGAGAAGTTTATGGATATTGGATTAAAAAGAGAAAAA
ACTGTCGAGGGCCATCTCTTATTCCATCAGTAAAACAAGAGAAGCGAGAT
GGTTCCAGCACAAATGATCCTTATGTGGCTTTTAGAAGGCGTACTGAAAA
AATGCAGACTCGAAAAAATCGCAAAAATGATGAAGCCTCTTACGAAAAAA
TGCTTAAGCTGCGACGAGATCTAAGTCGAGCTGTTACTATTCTAGAGATG
ATAAAAAGAAGAGAAAAAAGTAAAAGAGAGCTATTGCACTTAACACTGGA
AATTATGGAAAAGAGGTATAATTTGGGCGACTACAATGGAGAGATCATGT
CTGAGGTTATGGCACAGAGACAGCCAATGAAACCTACTTATGCCATCCCC
ATCATCCCTATTACTAATAGCAGTCAATTTAAACACCAGGAAGCAATGGA
TGTGAAGGAGTTCAAAGTTAATAAGCAAGATAAAGCCGATCTTATCCGAC
CGAAACGGAAATATGAAAAGAAGCCCAAAGTCTTACCATCGTCTGCCGCT
GCTACTCCCCAACAGACGAGTCCTGCTGCACTGCCAGTCTTCAATGCTAA
AGATCTGAATCAGTATGACTTTCCCAGCTCAGACGAAGAACCTCTCTCCC
AGGTTTTGTCTGGCTCTTCGGAAGCTGAGGAAGACAATGATCCTGATGGT
CCTTTTGCTTTCCGTAGGAAAGCAGGCTGTCAGTACTATGCTCCTCACTT
AGACCAAACTGGCAACTGGCCTTGGACTAGTCCTAAAGATGGAGGATTAG
GGGATGTGCGATATAGATACTGCTTAACTACTCTCACCGTACCCCAAAGG
TGTATTGGATTTGCACGAAGACGGGTTGGGCGCGGTGGAAGGGTCTTACT
GGACAGAGCTCATTCAGACTATGACAGTGTGTTTCACCATCTGGATTTGG
AAATGCTTTCCTCACCACAACATTCTCCAGTCAATCAGTTTGCCAATACC
TCAGAAACAAATACCTCGGACAAATCTTTCTCTAAAGACCTCAGTCAGAT
ACTAGTCAATATCAAATCATGTAGATGGCGGCATTTTAGGCCTCGGACAC
CATCCCTACATGACAGTGACAATGATGAACTCTCCTGTAGAAAATTATAT
AGGAGTATAAACCGAACAGGAACAGCACAACCTGGGACCCAGACATGCAG
TACCTCTACGCAAAGTAAAAGTAGCAGTGGTTCAGCACACTTTGCATTTA
CAGCCGAACAATACCAGCAACATCAACAGCAACTGGCACTCATGCAGAAA
CAGCAGCTTGCACAAATTCAGCAACAGCAAGCAAATAGTAATTCCTCCAC
CAACACATCACAGAACCTTGCATCTAACCAGCAGAAAAGTGGCTTTCGCC
TGAATATACAGGGTTTAGAAAGAACACTACAGGGTTTTGTTTCTAAGACT
TTGGATTCTGCTAGTGCACAGTTTGCTGCTTCTGCTTTGGTGACATCAGA
ACAACTGATGGGATTCAAGATGAAGGATGATGTGGTGCTTGGAATCGGGG
TGAATGGCGTCCTTCCAGCCTCAGGAGTATACAAGGGCTTACACCTCAGT
AGTACTACACCAACAGCACTTGTACATACAAGTCCATCAACGGCAGGTTC
AGCTTTGTTACAGCCTTCAAATATTACACAGACTTCAAGTTCCCACAGTG
CACTGAGTCATCAAGTAACTGCTGCCAATTCTGCAACAACTCAGGTTCTG
ATTGGGAACAACATTCGATTAACTGTACCTTCATCAGTTGCCACTGTAAA
CTCTATTGCCCCAATAAATGCACGACATATACCTAGGACTTTAAGTGCTG
TTCCATCATCTGCCTTAAAGCTGGCCGCTGCAGCAAACTGTCAAGTTTCC
AAGGTCCCATCTTCATCCTCTGTAGATTCAGTTCCAAGGGAAAATCATGA
ATCAGAAAAGCCAGCACTGAACAACATAGCAGACAACACAGTAGCGATGG
AGGTGACGTAG
EZH1

SEQ ID NO:77

>gi|4503622|ref|NM_001991.1|Homo sapiens enhancer
of zeste homolog 1 (Drosophila) (EZH1), mRNA
GAGGCTGGACACCTGTTCTGCTGTTGTGTCCTGCCATTCTCCTGAAGAAC
AGAGGCACACTGTAAAACCCAACACTTCCCCTTGCATTCTATAAGATTAC
AGCAAGATGGAAATACCAAATCCCCCTACCTCCAAATGTATCACTTACTG
GAAAAGAAAAGTGAAATCTGAATACATGCGACTTCGACAACTTAAACGGC
TTCAGGCAAATATGGGTGCAAAGGCTTTGTATGTGGCAAATTTTGCAAAG
GTTCAAGAAAAAACCCAGATCCTCAATGAAGAATGGAAGAAGCTTCGTGT
CCAACCTGTTCAGTCAATGAAGCCTGTGAGTGGACACCCTTTTCTCAAAA
AGTGTACCATAGAGAGCATTTTCCCGGGATTTGCAAGCCAACATATGTTA
ATGAGGTCACTGAACACAGTTGCATTGGTTCCCATCATGTATTCCTGGTC
CCCTCTCCAACAGAACTTTATGGTAGAAGATGAGACGGTTTTGTGCAATA
TTCCCTACATGGGAGATGAAGTGAAAGAAGAAGATGAGACTTTTATTGAG
GAGCTGATCAATAACTATGATGGGAAAGTCCATGGTGAAGAAGAGATGAT
CCCTGGATCCGTTCTGATTAGTGATGCTGTTTTTCTGGAGTTGGTCGATG
CCCTGAATCAGTACTCAGATGAGGAGGAGGAAGGGCACAATGACACCTCA
GATGGAAAGCAGGATGACAGCAAAGAAGATCTGCCAGTAACAAGAAAGAG
AAAGCGACATGCTATTGAAGGCAACAAAAAGAGTTCCAAGAAACAGTTCC
CAAATGACATGATCTTCAGTGCAATTGCCTCAATGTTCCCTGAGAATGGT
GTCCCAGATGACATGAAGGAGAGGTATCGAGAACTAACAGAGATGTCAGA
CCCCAATGCACTTCCCCCTCAGTGCACACCCAACATCGATGGCCCCAATG
CCAAGTCTGTGCAGCGGGAGCAATCTCTGCACTCCTTCCACACACTTTTT
TGCCGGCGCTGCTTTAAATACGACTGCTTCCTTCACCCTTTTCATGCCAC
CCCTAATGTATATAAACGCAAGAATAAAGAAATCAAGATTGAACCAGAAC
CATGTGGCACAGACTGCTTCCTTTTGCTGGAAGGAGCAAAGGAGTATGCC
ATGCTCCACAACCCCCGCTCCAAGTGCTCTGGTCGTCGCCGGAGAAGGCA
CCACATAGTCAGTGCTTCCTGCTCCAATGCCTCAGCCTCTGCTGTGGCTG
AGACTAAAGAAGGAGACAGTGACAGGGACACAGGCAATGACTGGGCCTCC
AGTTCTTCAGAGGCTAACTCTCGCTGTCAGACTCCCACAAAACAGAAGGC
TAGTCCAGCCCCACCTCAACTCTGCGTAGTGGAAGCACCCTCGGAGCCTG
TGGAATGGACTGGGGCTGAAGAATCTCTTTTTCGAGTCTTCCATGGCACC
TACTTCAACAACTTCTGTTCAATAGCCAGGCTTCTGGGGACCAAGACGTG
CAAGCAGGTCTTTCAGTTTGCAGTCAAAGAATCACTTATCCTGAAGCTGC
CAACAGATGAGCTCATGTACCCCTCACAGAAGAAGAAAAGAAAGCACAGA
TTGTGGGCTGCACACTGCAGGAAGATTCAGCTGAAGAAAGATAACTCTTC
CACACAAGTGTACAACTACCAACCCTGCGACCACCCAGACCGCCCCTGTG
ACAGCACCTGCCCCTGCATCATGACTCAGAATTTCTGTGAGAAGTTCTGC
CAGTGCAACCCAGACTGTCAGAATCGTTTCCCTGGCTGTCGCTGTAAGAC
CCAGTGCAATACCAAGCAATGTCCTTGCTATCTGGCAGTGCGAGAATGTG
ACCCTGACCTGTGTCTCACCTGTGGGGCCTCAGAGCACTGGGACTGCAAG
GTGGTTTCCTGTAAAAACTGCAGCATCCAGCGTGGACTTAAGAAGCACCT
GCTGCTGGCCCCCTCTGATGTGGCCGGATGGGGCACCTTCATAAAGGAGT
CTGTGCAGAAGAACGAATTCATTTCTGAATACTGTGGTGAGCTCATCTCT
CAGGATGAGGCTGATCGACGCGGAAAGGTCTATGACAAATACATGTCCAG
CTTCCTCTTCAACCTCAATAATGATTTTGTAGTGGATGCTACTCGGAAAG
GAAACAAAATTCGATTTGCAAATCATTCAGTGAATCCCAACTGTTATGCC
AAAGTGGTCATGGTGAATGGAGACCATCGGATTGGGATCTTTGCCAAGAG
GGCAATTCAAGCTGGCGAAGAGCTCTTCTTTGATTACAGGTACAGCCAAG
CTGATGCTCTCAAGTACGTGGGGATCGAGAGGGAGACCGACGTCCTTTAG
CCCTCCCAGGCCCCAACGGCAGCACTTATGGTAGCGGCACTGTCTTGGCT
TTCGTGCTCACACCACTGCTGCTCGAGTCTCCTGCACTGTGTCTCCCACA
CTGAGAAACCCCCCAACCCACTCCCCCTGTAGTGAGGCCTCTGCCATGTC
CAGAGGGCACAAAACTGTCTCAATGAGAGGGGAGACAGAGGCAGCTAGGG
CTTGGTCTCCCAGGACAGAGAGTTACAGAAATGGGAGACTGTTT
EZH2

SEQ ID NO:78

gi|4758323|ref|NM_004456.1|Homo sapiens enhancer
of zeste homolog 2 (Drosophila) (EZH2), mRNA
GAATTCCGGGCGACGCGCGGGAACAACGCGAGTCGGCGCGCGGGACGAAG
AATAATCATGGGCCAGACTGGGAAGAAATCTGAGAAGGGACCAGTTTGTT
GGCGGAAGCGTGTAAAATCAGAGTACATGCGACTGAGACAGCTCAAGAGG
TTCAGACGAGCTGATGAAGTAAAGAGTATGTTTAGTTCCAATCGTCAGAA
AATTTTGGAAAGAACGGAAATCTTAAACCAAGAATGGAAACAGCGAAGGA
TACAGCCTGTGCACATCCTGACTTCTGTGAGCTCATTGCGCGGGACTAGG
GAGTGTTCGGTGACCAGTGACTTGGATTTTCCAACACAAGTCATCCCATT
AAAGACTCTGAATGCAGTTGCTTCAGTACCCATAATGTATTCTTGGTCTC
CCCTACAGCAGAATTTTATGGTGGAAGATGAAACTGTTTTACATAACATT
CCTTATATGGGAGATGAAGTTTTAGATCAGGATGGTACTTTCATTGAAGA
ACTAATAAAAAATTATGATGGGAAAGTACACGGGGATAGAGAATGTGGGT
TTATAAATGATGAAATTTTTGTGGAGTTGGTGAATGCCCTTGGTCAATAT
AATGATGATGACGATGATGATGATGGAGACGATCCTGAAGAAAGAGAAGA
AAAGCAGAAAGATCTGGAGGATCACCGAGATGATAAAGAAAGCCGCCCAC
CTCGGAAATTTCCTTCTGATAAAATTTTGGAGGCCATTTCCTCAATGTTT
CCAGATAAGGGCACAGCAGAAGAACTAAAGGAAAAATATAAAGAACTCAC
CGAACAGCAGCTCCCAGGCGCACTTCCTCCTGAATGTACCCCCAACATAG
ATGGACCAAATGCTAAATCTGTTCAGAGAGAGCAAAGCTTACACTCCTTT
CATACGCTTTTCTGTAGGCGATGTTTTAAATATGACTGCTTCCTACATCC
TTTTCATGCAACACCCAACACTTATAAGCGGAAGAACACAGAAACAGCTC
TAGACAACAAACCTTGTGGACCACAGTGTTACCAGCATTTGGAGGGAGCA
AAGGAGTTTGCTGCTGCTCTCACCGCTGAGCGGATAAAGACCCCACCAAA
ACGTCCAGGAGGCCGCAGAAGAGGACGGCTTCCCAATAACAGTAGCAGGC
CCAGCACCCCCACCATTAATGTGCTGGAATCAAAGGATACAGACAGTGAT
AGGGAAGCAGGGACTGAAACGGGGGGAGAGAACAATGATAAAGAAGAAGA
AGAGAAGAAAGATGAAACTTCGAGCTCCTCTGAAGCAAATTCTCGGTGTC
AAACACCAATAAAGATGAAGCCAAATATTGAACCTCCTGAGAATGTGGAG
TGGAGTGGTGCTGAAGCCTCAATGTTTAGAGTCCTCATTGGCACTTACTA
TGACAATTTCTGTGCCATTGCTAGGTTAATTGGGACCAAAACATGTAGAC
AGGTGTATGAGTTTAGAGTCAAAGAATCTAGCATCATAGCTCCAGCTCCC
GCTGAGGATGTGGATACTCCTCCAAGGAAAAAGAAGAGGAAACACCGGTT
GTGGGCTGCACACTGCAGAAAGATACAGCTGAAAAAGGACGGCTCCTCTA
ACCATGTTTACAACTATCAACCCTGTGATCATCCACGGCAGCCTTGTGAC
AGTTCGTGCCCTTGTGTGATAGCACAAAATTTTTGTGAAAAGTTTTGTCA
ATGTAGTTCAGAGTGTCAAAACCGCTTTCCGGGATGCCGCTGCAAAGCAC
AGTGCAACACCAAGCAGTGCCCGTGCTACCTGGCTGTCCGAGAGTGTGAC
CCTGACCTCTGTCTTACTTGTGGAGCCGCTGACCATTGGGACAGTAAAAA
TGTGTCCTGCAAGAACTGCAGTATTCAGCGGGGCTCCAAAAAGCATCTAT
TGCTGGCACCATCTGACGTGGCAGGCTGGGGGATTTTTATCAAAGATCCT
GTGCAGAAAAATGAATTCATCTCAGAATACTGTGGAGAGATTATTTCTCA
AGATGAAGCTGACAGAAGAGGGAAAGTGTATGATAAATACATGTGCAGCT
TTCTGTTCAACTTGAACAATGATTTTGTGGTGGATGCAACCCGCAAGGGT
AACAAAATTCGTTTTGCAAATCATTCGGTAAATCCAAACTGCTATGCAAA
AGTTATGATGGTTAACGGTGATCACAGGATAGGTATTTTTGCCAAGAGAG
CCATCCAGACTGGCGAAGAGCTGTTTGTTGATTACAGATACAGCCAGGCT
GATGCCCTGAAGTATGTCGGCATCGAAAGAGAAATGGAAATCCCTTGACA
TCTGCTACCTCCTCCCCCTCCTCTGAAACAGCTGCCTTAGCTTCAGGAAC
CTCGAGTACTGTGGGCAATTTAGAAAAAGAACATGCAGTTTGAAATTCTG
AATTTGCAAAGTACTGTAAGAATAATTTATAGTAATGAGTTTAAAAATCA
ACTTTTTATTGCCTTCTCACCAGCTGCAAAGTGTTTTGTACCAGTGAATT
TTTGCAATAATGCAGTATGGTACATTTTTCAACTTTGAATAAAGAATACT
TGAACTTGAAAAAAAAAAAAAAAAAA
BMI1

SEQ ID NO:79

>gi|4885094|ref|NM_005180.1|Homo sapiens B
lymphoma Mo-MLV insertion region (mouse) (BMI1),
mRNA
GAGAGGCAGAGATCGGGGCGAGACAATGGGGATGTGGGCGCGGGAGCCCC
GTTCCGGCTTAGCAGCACCTCCCAGCCCCGCAGAATAAAACCGATCGCGC
CCCCTCCGCGCGCGCCCTCCCCCGAGTGCGGAGCGGGAGGAGGCGGCGGC
GGCCGAGGAGGAGGAGGAGGAGGCCCCGGAGGAGGAGGCGTTGGAGGTCG
AGGCGGAGGCGGAGGAGGAGGAGGCCGAGGCGCCGGAGGAGGCCGAGGCG
CCGGAGCAGGAGGAGGCCGGCCGGAGGCGGCATGAGACGAGCGTGGCGGC
CGCGGCTGCTCGGGGCCGCGCTGGTTGCCCATTGACAGCGGCGTCTGCAG
CTCGCTTCAAGATGGCCGCTTGGCTCGCATTCATTTTCTGCTGAACGACT
TTTAACTTTCATTGTCTTTTCCGCCCGCTTCGATCGCCTCGCGCCGGCTG
CTCTTTCCGGGATTTTTTATCAAGCAGAAATGCATCGAACAACGAGAATC
AAGATCACTGAGCTAAATCCCCACCTGATGTGTGTGCTTTGTGGAGGGTA
CTTCATTGATGCCACAACCATAATAGAATGTCTACATTCCTTCTGTAAAA
CTTGTATTGTTCGTTACCTGGAGACCAGCAAGTATTGTCCTATTTGTGAT
GTCCAAGTTCACAAGACCAGACCACTACTGAATATAAGGTCAGATAAAAC
TCTCCAAGATATTGTATACAAATTAGTTCCAGGGCTTTTCAAAAATGAAA
TGAAGAGAAGAAGGGATTTTTATGCAGCTCATCCTTCTGCTGATGCTGCC
AATGGCTCTAATGAAGATAGAGGAGAGGTTGCAGATGAAGATAAGAGAAT
TATAACTGATGATGAGATAATAAGCTTATCCATTGAATTCTTTGACCAGA
ACAGATTGGATCGGAAAGTAAACAAAGACAAAGAGAAATCTAAGGAGGAG
GTGAATGATAAAAGATACTTACGATGCCCAGCAGCAATGACTGTGATGCA
CTTAAGAAAGTTTCTCAGAAGTAAAATGGACATACCTAATACTTTCCAGA
TTGATGTCATGTATGAGGAGGAACCTTTAAAGGATTATTATACACTAATG
GATATTGCCTACATTTATACCTGGAGAAGGAATGGTCCACTTCCATTGAA
ATACAGAGTTCGACCTACTTGTAAAAGAATGAAGATCAGTCACCAGAGAG
ATGGACTGACAAATGCTGGAGAACTGGAAAGTGACTCTGGGAGTGACAAG
GCCAACAGCCCAGCAGGAGGAGTTCCCTCCACCTCTTCTTGTTTGCCTAG
CCCCAGTACTCCAGTGCAGTCTCCTCATCCACAGTTTCCTCACATTTCCA
GTACTATGAATGGAACCAGCAACAGCCCCAGCGGTAACCACCAATCTTCT
TTTGCCAATAGACCTCGAAAATCATCAGTAAATGGGTCATCAGCAACTTC
TTCTGGTTGATACCTGAGACTGTTAAGGAAAAAAATTTTAAACCCCTGAT
TTATATAGATATCTTCAGCCATTACGACTTTCTAGAGCTAATACATGTGA
CTATCGTCCAATTTGCTTTCTTTTGTAGTGACATTAAATTTGGCTATAAA
AGATGGACTACATGTGATACTCCTGTCCGTCTTGGTTCAAAAGAAAGATT
GTTGTTATAAAGAATTGGTTTCTTGGAAAGCAGGCAAGACTTTTTCTCTG
TGTTAGGAAAGATGGGAAATGGTTTCTGTAACCATTGTTTGGATTTGGAA
GTACTCTGCAGTGGACATAAGCATTGGGCCATAGTTTGTTAATCTCAACT
AACGCCTACATTACATTCTCCTTGATCGTTCTTGTTATTACGCTGTTTTG
TGAACCTGTAGAAAAACAAGTGCTTTTTATCTTGAAATTCAACCAACGGA
AAGAATATGCATAGAATAATGCATTCTATGATGCCATGTCACTGTGAATA
ACGATTTCTTGCAGCTATTTAGCCATTTTGATTGCTGTTTGATTTATACT
TCTCTGTTGCTACGCAAAACCGATCAAAGAAAAGTGAACTTCAGTTTTAC
AATCTGTATGCCTAAAAGCGGGTACTACCGTTTATTTTACTGACTTGTTT
AAATGATTCGCTTTTGTAAGAATCAGATGGCATTATGCTTGTTGTACAAT
GCCATATTGGTATATGACATAACAGGAAACAGTATTGTATGATATATCTA
TAAATGCTATAAAGAAATATTGTGTTTCATGCATTCAGAAATGATTGTTA
AAATTCTCCCAACTGGTTCGACCTTTGCAGATACCCATAACCTATGTTGA
GCCTTGCTTACCAGCAAAGAATATTTTTAATGTGGATATCTAATTCTAAA
GTCTGTTCCATTAGAAGCAATTGGCACATCTTTCTATACTTTATATACTT
TTCTCCAGTAATACATGTTTACTTTAAAAATTGTTGCAGTGAAGAAAAAC
CTTTAACTGAGAAATATGGAAACCGTCTTAATTTTCCATTGGCTATGATG
GAATTAATATTGTATTTTAAAAATGCATATTGATCACTATAATTCTAAAA
CAATTTTTTAAATAAACCAGCAGGTTGCTAAAAGAAGGCATTTTATCTAA
AGTTATTTTAATAGGTGGTATAGCAGTAATTTTAAATTTAAGAGTTGCTT
TTACAGTTAACAATGGAATATGCCTTCTCTGCTATGTCTGAAAATAGAAG
CTATTTATTATGAGCTTCTACAGGTATTTTTAAATAGAGCAAGCATGTTG
AATTTAAAATATGAATAACCCCACCCAACAATTTTCAGTTTATTTTTTGC
TTTGGTCGAACTTGGTGTGTGTTCATCAGTTATTTGTGAGGGTGTTTATT
CTATATGAATATTGTTTCATGTTTGTAGGGAAATTGTAGCTAAACATTTC
ATTGTCCCCAGTCTGCAAAAGAAGCACAATTCTATTGCTTTGTCTTGCTT
ATAGTCATTAAATCATTACTTTTACATATATTGCTGTTACTTCTGCTTTC
TTTAAAAATATAGTAAAGGATGTTTTATGAAGTCACAAGATACATATATT
TTTATTTTGACCTAAATTTGTACAGTCCCATTGTAAGTGTTGTTTCTAAT
TATAGATGTAAAATGAAATTTCATTTGTAATTGGAAAAAATCCAATAAAA
AGGATATTCATTTAGAAAATAGCTAAGATCTTTAATAAAAATTTGATATG
AAA
ZNF144 (MEL18)

SEQ ID NO:80

>gi|6005963|ref|NM_007144.1|Homo sapiens zinc
finger protein 144 (Mel-18) (ZNF144), MRNA
GAGAGCCCGAACAGGAAGAGGGTACAGCTTTGTGCAGGTCACATGCCCAC
TGCAGCCCTCCAGCCTCTGGTCCCCAGAGCGGACTTTGGAAGCTGAACTG
CTTTTGTTGCTGGAAGACTTATGTTATAATTTACCCTGGGTGGACCAGGG
TCGTACAAAAGGGCAACGCTCCCCAGTCCCCCCACTCCCGACCCCGGAAT
CATGCATCGGACTACACGGATCAAAATCACAGAGCTGAACCCCCACCTCA
TGTGTGCCCTCTGCGGGGGGTACTTCATCGACGCCACCACTATCGTGGAG
TGCCTGCATTCCTTCTGCAAAACCTGCATCGTGCGCTACCTGGAGACCAA
CAAATACTGCCCCATGTGTGACGTGCAGGTCCATAAAACCCGGCCGCTGC
TGAGCATCAGGTCTGACAAAACACTTCAAGACATTGTCTACAAATTGGTC
CCTGGGCTTTTTAAAGATGAGATGAAACGGCGGCGGGATTTCTATGCAGC
GTACCCCCTGACGGAGGTCCCCAACGGCTCCAATGAGGACCGCGGCGAGG
TCTTGGAGCAGGAGAAGGGGGCTCTGAGTGATGATGAGATTGTCAGCCTC
TCCATCGAATTCTACGAAGGTGCCAGGGACCGGGATGAGAAGAAGGGCCC
CCTGGAGAATGGGGATGGGGACAAAGAGAAAACAGGGGTGCGCTTCCTGC
GATGCCCAGCAGCCATGACCGTCATGCATCTTGCCAAGTTTCTCCGCAAC
AAGATGGATGTGCCCAGCAAGTACAAGGTGGAGGTTCTGTACGAGGACGA
GCCACTGAAGGAATACTACACCCTCATGGACATCGCCTACATCTACCCCT
GGCGGCGGAACGGGCCTCTCCCCCTCAAGTACCGTGTCCAGCCAGCCTGC
AAGCGGCTCACCCTAGCCACGGTGCCCACCCCCTCCGAGGGCACCAACAC
CAGCGGGGCGTCCGAGTGTGAGTCAGTCAGCGACAAGGCTCCCAGCCCTG
CCACCCTGCCAGCCACCTCCTCCTCCCTGCCCAGCCCAGCCACCCCATCC
CATGGCTCTCCCAGTTCCCATGGGCCTCCAGCCACCCACCCTACCTCCCC
CACTCCCCCTTCGACAGCCAGTGGGGCCACCACAGCTGCCAACGGGGGTA
GCTTGAACTGCCTGCAGACACCATCCTCCACCAGCAGGGGGCGCAAGATG
ACTGTCAACGGCGCTCCCGTGCCCCCCTTAACTTGAGGCCAGGGACCCTC
TCCCTTCTTCCAGCCAAGCCTCTCCACTCCTTCCACTTTTTCTGGGCCCT
TTTTTCCACTTCTTCTACTTTCCCCAGCTCTTCCCACCTTGGGGGTGGGG
GGCGGGTTTTATAAATAAATATATATATATATGTACATAGGAAAAACCAA
ATATACATACTTATTTTCTATGGACCAACCAGATTAATTTAAATGCCACA
GGAAACAAACTTTATGTGTGTGTGTATGTGTGGAAAATGGTGTTCATTTT
TTTTGGGGGGGGTCTTGTGTAATTTGCTGTTTTTGGGGGTGCCTGGAGAT
GAACTGGATGGGCCACTGGAGTCTCAATAAAGCTCTGCACCATCCTCGCT
GTTTCCCAAGGCAGGTGGTGTGTTGGGGGCCCCTTCAGACCCAAAGCTTT
AGGCATGATTCCAACTGGCTGCATATAGGAGTCAGTTAGAATTGTTTCTT
TCTCTCCCCGTTTCTCTCCCCATCTTGGCTGCTGTCCTGCCTCTGACCAG
TGGCCGCCCCCCGCGTTGTTGAATGTCCAGAAATTGCTAAGAACAGTGCC
TTTTACAAATGCAGTTTATCCCTGGTTCTGAGGAGCAAGTGCAGGGTGGA
GGTGGCACCTGCATCACCTCCTCCTCTTGCAGTGGAAACTTTGTGCAAAG
AATAGATAGTTCTGCCTCTTTTTTTTTTTTTTCCTGTGTGTGTGGCCTTT
GCATCATTTATCTTGTGGAAAAGAAGATTCAGGCCCTGAGAGGTCTCAGC
TCTTGGAGGAGGGCTAAGGCTTTAGCATTGTGAAGCGCTGCACCCCCACC
AACCTTACCCTCACCGGGGAACCCTCACTAGCAGGACTGGTGGTGGAGTC
TCACCTGGGGCCTAGAGTGGAAGTGGGGGTGGGTTAACCTCACACAAGCA
CAGATCCCAGACTTTGCCAGAGGCAAACAGGGAATTCCGCCGATACTGAC
GGGCTCCAGGAGTCGTCGCCACACTCG
SCML1

SEQ ID NO:81

>gi|5803158|ref|NM_006746.1|Homo sapiens sex comb
on midleg-like 1 (Drosophila) (SCML1), mRNA
ATCACTGGTCTCGCGTGCGCGTGACCAGGCCCGGTTTCCGGTGCCAGGAC
CTTTCCGAAGCGTCGAGTGGCCTAACGGTCACAGCTGTCGCCCATCGGAG
AGGCAGGACTACTGCGAGCAGTTTTACCGCGACCTCCGGAGGCCGGCGTG
ACAGGCTCTGTCACTAAAATAGGAACCGAATATTGTATCTGACGCATCCT
GTAATACTGAAGAGCAACTGAAGACAGTTGATGATGTCCTTATTCATTGC
CAGGTTATATATGATGCTCTGCAAAACCTGGATAAGAAGATTGATGTGAT
TCGTAGAAAGGTTTCAAAAATCCAACGTTTCCATGCGAGATCCCTGTGGA
CAAATCATAAGCGATATGGATATAAAAAGCATTCTTACCGGCTTGTTAAA
AAGCTTAAACTCCAGAAAATGAAGAAAAATGAGGTTTACGAGACATTCTC
CTACCCTGAAAGTTACAGCCCCACTTTACCAGTGTCAAGGCGTGAGAATA
ATTCCCCGAGCAACCTTCCAAGGCCATCCTTTTGCATGGAAGAATACCAG
CGAGCTGAGCTGGAGGAGGACCCGATCCTCAGCCGCACTCCGAGTCCAGT
GCATCCCTCAGATTTCTCTGAGCATAATTGTCAGCCGTATTATGCATCTG
ATGGTGCAACGTATGGTTCTTCTTCAGGGCTCTGCCTTGGCAACCCTCGG
GCTGACAGCATCCACAACACTTACTCAACTGACCATGCTTCTGCAGCACC
ACCTTCAGTTACAAGGTCACCAGTTGAAAATGACGGTTACATAGAGGAAG
GAAGCATCACTAAGCACCCTTCAACCTGGTCGGTGGAAGCAGTGGTCCTA
TTTCTAAAACAAACAGATCCTCTTGCATTATGCCCTCTTGTCGACCTCTT
CAGAAGCCATGAAATTGACGGGAAGGCTCTGCTCCTACTCACGAGTGACG
TGTTGCTGAAGCACTTGGGGGTGAAGCTGGGAACGGCTGTGAAGCTATGC
TACTACATTGACCGACTTAAACAAGGAAAATGCTTTGAAAATTGAAAAAA
TCCTTGTGCAAATTTAGATTGGGCCAACTTCTAGAGGCACCAATGCCTTC
TTAGTGTGGAATCATTTTTCTGCCCTTTAGTCGTTTTTGTTTTGTAGAAA
GTATCTCTCAAAATATATTATAGCTAGAATTGTAGAACTATGTTATAGTC
CAGTCTACTTCTTTAAAAACCATTTAAACTGCTAGATAGTATTAGAATAG
TCCAATAGAAAATTCATTCTTTATAGGTCTTTAAAAATTACTTTTATTAT
ATTGTTTACAAATATATTTCATGCAAGAAACAGAAAAAAAAAAAAACCCT
TTGATTCTGGTTCATCTCGATACAGAGAACCAAAACAGCTAAGAGAGGTA
TTATCAGGGTTGACAACTCCTATGATTGAATCTATGGGAATTATTCCTCA
GAAGAGAATTTAAAGGTGTACCCATATATATCTCTTTCTGGAGTATTTTA
TCTGTCTGATGTTGCAGTATTCTACAAGTTTCCAGAAAGAGAATAGCCAT
ATAAATTATTTTCCTTTCTGCTATTATTTCTCTATATGTTTTATTTATTC
AGATTTAGAGTAAAAAATAAGCATATAAACTTTTATTATGTGCTCTTAAC
AGTTTTAAGATAAACTATAGGATAGATAGAATGGTTATTTTATGCAAGAA
ATATTGTACCGCAAGGGTGGTTTGGATGAAGTCTGACTACTTTTTTTCAA
ACAAACTATTATATTAAAACTGTCATATTTTGGCTAAGTTTGGACCTATA
ACTACACTTTCATTGTTTGCATCTCTCTATGAAGATACGTCTGTCCAAAC
TTTTAAAAGGCATAACTGTATTTTATGTGTTTATTCTTTATATAGATAGT
ATTTTATATTTTATTCTCACCCGAAGTATTCACACAATCTTTTTAAAAAA
AATTTGAAATGGCATTTTGTATTGCCACAGAGGTAGGATGAGCCATATAT
TAGTGAAATGTTTTATTTTGTAAAATATAAATGGATTATTTGCCATCATT
AGTACCTCTCAACTTACTTTTTAGAGGACAAGAAACAATCTGTAGATTGG
TTTCCATACAGGGAAGTTCTCCGTCCTATGCAATGTTTCTAATTAATTTG
CTTAATTCTGAGCCATTAATCCTGCTACACTTTGAATGATACATTAATTC
AGACTAATCTTTGGGGGCTTTATTTTGTAAGTTAGAACTTTCAAGGGAAA
CATGTTCAACACTATTATTTTGTTATAAATTTATAACTTTGTTATTACAT
TGTGTAACAAATATAAGGTTTACGAGCTATGAGAATTGGTGCTATCACCA
TTAGCTATTTGCTGTAATGTCAAGAAAATGTTCACCAGATGCAAGAATGT
ACCTTTTCTTTTTAGAAAGCCAAATGTACTTTAGACATGAATGCAACTAT
TTAAAGAATAGCTTCATCAATGTTATTCCTTACATGTCATAAGATTCTTA
CTTAAACTTGGTCTTCTTTCAAATTGTTTGTATGAAGATGCTGTACCCAC
TTGAACAGTCCTCAGGTGTTTACATAAATACTATGTTTTACAGTTTTCAT
ATTTTAAAATATTAATAAAGTTAAATCACAATAGT
SCML2

SEQ ID NO:82

>gi|5174668|ref|NM_006089.1|Homo sapiens sex comb
on midleg-like 2 (Drosophila) (SCML1), mRNA
GAAGCTCGGTGCCGGCTCGCGCGATCGGTGGGACAGAATTTCGTTGTTTT
CACCGACGAGACTGGAGGAAACAACACCAAATAGGGATACCATGGGACAA
ACAGTGAATGAAGATTCCATGGATGTCAAGAAGGAGAATCAAGAGAAAAC
TCCTCAGTCAAGTACATCTTCTGTACAAAGGGATGATTTCCACTGGGAGG
AGTATTTGAAAGAGACTGGGTCTATAAGTGCTCCTTCAGAGTGCTTCCGT
CAGTCTCAGATTCCACCTGTGAATGATTTCAAAGTTGGTATGAAATTGGA
AGCCCGTGACCCTCGCAATGCCACTTCAGTATGTATTGCTACGGTTATTG
GAATTACTGGGGCCAGGTTACGGTTACGACTGGATGGTAGTGACAACAGA
AATGATTTTTGGAGGCTTGTCGATTCCCCAGACATACAACCTGTTGGGAC
ATGTGAAAAGGAAGGAGACTTACTTCAACCTCCACTAGGGTACCAGATGA
ATACATCCTCCTGGCCGATGTTCCTCTTAAAGACACTAAATGGGTCTGAA
ATGGCATCTGCCACATTATTTAAGAAGGAACCACCAAAGCCCCCACTAAA
TAATTTTAAAGTGGGGATGAAACTGGAAGCTATTGACAAAAAGAACCCGT
ATCTCATCTGTCCTGCGACCATTGGAGATGTTAAAGGGGATGAAGTTCAT
ATCACATTTGATGGCTGGAGTGGAGCTTTTGATTACTGGTGCAAGTATGA
TTCTCGAGATATTTTCCCAGCTGGGTGGTGTCGCCTGACAGGAGATGTAT
TACAACCCCCAGGAACTAGTGTTCCTATTGTAAAGAATATAGCAAAAACA
GAGTCTTCTCCTTCCGAAGCAAGCCAGCATTCAATGCAGTCTCCACAGAA
AACTACTCTAATATTACCAACACAGCAGGTCAGGAGATCAAGTCGAATTA
AACCACCTGGACCTACTGCAGTCCCCAAAAGGAGCAGTTCTGTTAAAAAT
ATCACACCAAGGAAAAAAGGTCCAAACTCAGGAAAAAAGGAAAAACCTTT
GCCCGTGATATGTTCTACATCTGCAGCTTCTCTAAAATCGCTGACCAGAG
ACCGTGGCATGTTATATAAAGATGTCGCTTCTGGGCCATGTAAAATAGTG
ATGTCTACAGTCTGTGTCTATGTAAACAAACATGGAAACTTTGGCCCTCA
TCTGGATCCCAAGAGAATCCAGCAGCTGCCTGACCACTTCGGCCCGGGCC
CGGTGAATGTGGTGCTTCGCCGGATTGTGCAGGCCTGTGTGGATTGTGCC
CTTGAAACTAAAACTGTTTTTGGATACCTGAAGCCAGATAATCGTGGAGG
AGAAGTGATAACTGCCTCCTTTGATGGGGAAACTCATTCCATCCAGCTCC
CTCCAGTGAACAGTGCATCATTTGCTCTTCGCTTTCTTGAGAACTTCTGC
CACAGTCTGCAGTGTGATAACCTTTTGAGTAGCCAGCCTTTTAGTTCTTC
CAGGGGTCATACTCACAGCTCTGCAGAGCATGATAAAAATCAGTCAGCAA
AAGAAGATGTAACAGAAAGGCAAAGCACCAAACGATCTCCTCAGCAAACT
GTACCATATGTTGTTCCTCTCTCTCCTAAGCTCCCCAAAACAAAGGAGTA
TGCGTCTGAAGGAGAACCATTGTTTGCTGGGGGAAGTGCCATTCCCAAAG
AGGAGAATCTTTCAGAAGATTCTAAGAGCTCATCACTAAATTCAGGAAAT
TATTTGAATCCTGCCTGTAGAAATCCTATGTATATTCATACTTCAGTCTC
CCAGGATTTTTCTCGAAGTGTGCCAGGCACCACAAGTTCACCACTAGTTG
GGGACATATCCCCCAAGAGCAGTCCCCATGAAGTTAAATTCCAAATGCAG
AGGAAAAGTGAAGCTCCAAGTTATATAGCTGTACCTGATCCCAGTGTCCT
GAAACAAGGCTTCTCTAAGGACCCTTCAACCTGGTCTGTGGATGAAGTGA
TACAGTTTATGAAACATACAGATCCTCAGATATCAGGCCCCCTCGCCGAC
CTCTTCAGGCAACATGAAATTGATGGGAAGGCTCTGTTCCTACTCAAGAG
TGATGTGATGATGAAGTATATGGGGCTGAAGCTGGGGCCAGCATTAAAGC
TGTGTTACTACATTGAAAAGCTTAAAGAAGGAAAATACAGTTAAAAAATG
TGTAAGTTTAGATTGGACATAATTCTCAGGTGTACTGTTAACATTTTAAT
TTAAAAGTATTTCTCTTAGCAGTTTTTGTTTTGTAGACAGTTCCCATAAA
AATATTTTATCAGAATTGCAGAACTGTAGTAACAGTTCAGTCAACTTTGT
TTTTTTCCTGGAGTCACCAACCAGCTTTGGGAGACACAGCCGCCACTCCC
CCAGTCTACTTCTTTAAAAAGCATTTAACAGGTTAGTATTGGCATATTCA
AATTGGCAGTTCTTTATGTCTTTTTAAATTTTCATTGTACAGTTTACAAA
TATACTTAATGTAGTTAACAGAGAAAAACCTTTGATTTTGGTTAACCTTT
ATATCTAGAACCAAAACAGCTAAATCCCAAAGGGGAAAATATCAGGGATT
GACAACTTCTATAATTAAATCCATGAGAATTTTTCCTCACTAGAGAATTT
AAAGGTGCACCTGTAGATATCATCTTTTCTCAGATATTTTGTGTGATACT
CTGTGGTGTTCTGTTCATGTTCTATCAGTATATCTAGAAAGGGAATAGCC
ATATAAATTATTTTCCTTTTATTATTTCTCTGTATGTTGTATTTGATCAT
ATTTAAAGGAAAAAAGCAAGCTTATAAGCTTTCATGAAGTGTTCTTACCA
GTTTTTGATAAATTTTTTAAATTATAGGATAGAATTGTCATTTTATGCAG
GAGATATTTATACTACGAGGGTTGTTTGGATGGAGTCAGATTAAATTTTT
TCAGTGAAATTCCTATTATTTTAAAACTTTCCATATTTTCACTACGCTTG
GACATTTAACTAGGCATTCTTTTCTTACATCTCTATATGAAGATACCTGT
GTCCAAAATTTTTGAAGATATATATTGTATGTGTTTATTCTTCATATGGT
ACTTTACCATATTTATATATTGTTTTATACCTGTAGGTTTACACACAAGT
AAATCTTTTTTTCTTGAATTTAATCTGGGCACTTTGCACTGCCACAGGGG
TGACGATGGAACTATGTATATAGTTAGGATGTTTTGATTTCGGTAAAAAA
TATATGTCCCATCGTTGCTATCACCAGTACCTCTCAGCTTACTCTTCAGG
GGATATGAAACAATCTGTAGATTGGGTTTCCATAACAGGGAAGTCCGTCC
TAATGCCAAGTTTCTAATTAATTTGCTTAGTTCTGAGCCATTTATTCTGC
TACACTTTGAAAGATATATTAGTTCTGACTTATTGTTGGGGCTTTATTTT
ATTTTTATTTTTTTGAGATGGAGTTTCACTCTTGTGCCCAGGCTGGAGTG
CAATAGCTCGATCTCGGCTCACTGCAACCTCGCCTCCCTGGTTCAAGCAA
TTCTCCTGCCTCAGCGATTACAGGAATGCACCACCACGCCCAGCTAATTT
TGTAGAGATGGGGTTTCTCCATGTTGGTCAGGCTGGTCTCAAACTCCTGA
CCTCAGGTGATCCGCCCACCTCGGCCTTGCAAAATGCTGGGATTACAGGC
ATGAGCCACCTGCCCAGCCATGTTTGGGGCTTTATTTTATAAGTTAGAAC
TTTGAAGAGGAAATGGTGCTATATGTTTATTGTTATTACTTTGTGTAACT
TTGATGTAATGTTTATAAGCTATGAGAATCAGTTATAAAAGTATTAGCCA
TTTGTTGTAAATGCCAATAAAATATTCACCAGGGGCAAGAATGTACATTT
TCTTTTTAGAAAACCAAATGTACTTTAGACATGAATGCAACTATTTAAAG
AATAGCTTCATTTATGTTATTCCTTACATGTCATAAGATTCTTACTTAAA
CTTGGTCTTCTTTCAAGTTGTTTGTATGAAGATGCTGTACCCACTTGAAC
AGTCCTCAGGTGTTTACATAAATACTATGTTTTACAGTTTTCATATTTTA
AAATATTAATAAAGTTAATCGCAACGATTC
RING1

SEQ ID NO:83

>gi|11863157|ref|NM_002931.2|Homo sapiens ring
finger protein 1 (RING1), mRNA
GGCTGCTGTTTCTAAAACCCCTTTCCCTCTAACCCACACCACCTTTCTAC
TCACTGATGCCTTCAGGAAGCCATAATGGATGGCACAGAGATTGCTGTTT
CCCCTCGGTCACTGCATTCAGAACTCATGTGCCCTATCTGCCTGGACATG
CTGAAGAATACGATGACCACCAAGGAGTGCCTCCACAGATTCTGCTCTGA
CTGCATTGTCACAGCCCTACGGAGCGGGAACAAGGAGTGTCCTACCTGCC
GAAAGAAGCTGGTGTCCAAGCGATCCCTACGGCCAGACCCCAACTTTGAT
GCCCTGATCTCTAAGATCTATCCTAGCCGGGAGGAATACGAGGCCCATCA
AGACCGAGTGCTTATCCGCCTGAGCCGCCTGCACAACCAGCAGGCATTGA
GCTCCAGCATTGAGGAGGGGCTACGCATGCAGGCCATGCACAGGGCCCAG
CGTGTGAGGCGGCCGATACCAGGGTCAGATCAGACCACAACGATGAGTGG
GGGGGAAGGAGAGCCCGGGGAGGGAGAAGGGGATGGAGAAGATGTGAGCT
CAGACTCCGCCCCTGACTCTGCCCCAGGCCCTGCTCCCAAGCGACCCCGT
GGAGGGGGCGCAGGGGGGAGCAGTGTAGGGACAGGGGGAGGCGGCACTGG
TGGGGTGGGTGGGGGTGCCGGTTCGGAAGACTCTGGTGACCGGGGAGGGA
CTCTGGGAGGGGGAACGCTGGGCCCCCCAAGCCCTCCTGGGGCCCCCAGC
CCCCCAGAGCCAGGTGGAGAAATTGAGCTCGTGTTCCGGCCCCACCCCCT
GCTCGTGGAGAAGGGAGAATACTGCCAGACGAGGTATGTGAAGACAACTG
GGAATGCCACAGTGGACCACCTCTCCAAGTACTTGGCCCTGCGCATTGCC
CTCGAGCGGAGGCAACAGCAGGAAGCAGGGGAGCCAGGAGGGCCTGGAGG
GGGCGCCTCTGACACCGGAGGACCTGATGGGTGTGGCGGGGAGGGTGGGG
GTGCCGGAGGAGGTGACGGTCCTGAGGAGCCTGCTTTGCCCAGCCTGGAG
GGCGTCAGTGAAAAGCAGTACACCATCTACATCGCACCTGGAGGCGGGGC
GTTCACGACGTTGAATGGCTCGCTGACCCTGGAGCTGGTGAATGAGAAAT
TCTGGAAGGTGTCCCGGCCACTGGAGCTGTGCTATGCTCCCACCAAGGAT
CCAAAGTGACCCCACCAGGGGACAGCCAGAGGAAGGGGACCATGGGGTAT
CCCTGTGTCCTGGTCTATCACCCCAGCTTCTTTGTCCCCCAGTACCCCCA
GCCCAGCCAGCCAATAAGAGGACACAAATGAGGACACGTGGCTTTTATAC
AAAGTATCTATATGAGATTCTTCTATATTGTACAGAGTGGGGCAAAACAC
GCCCCCATCTGCTGCCTTTTCTATTGCCCTGCAACGTCCCATCTATACGA
GGTGTTGGAGAAGGTGAAGAACCCTCCCATTCACGCCCGCCTACCAACAA
CAAACGTGCTTTTTTCCTCTTTGAAAAAA
RYBP

SEQ ID NO:84

>gi|6912639|ref|NM_012234.1|Homo sapiens Ring1 and
YY1 binding protein (RYBP), mRNA
CCCGGACGGCGTTTCTCCTCCGAGCGGCGCCGGTTTCGGCTTGGGGGGGG
CGGGGTACAGCCCATCCATGACCATGGGCGACAAGAAGAGCCCGACCAGG
CCAAAAAGACAAGCGAAACCTGCCGCAGACGAAGGGTTTTGGGATTGTAG
CGTCTGCACCTTCAGAAACAGTGCTGAAGCCTTTAAATGCAGCATCTGCG
ATGTGAGGAAAGGCACCTCCACCAGAAAACCTCGGATCAATTCTCAGCTG
GTGGCACAACAAGTGGCACAACAGTATGCCACCCCACCACCCCCTAAAAA
GGAGAAGAAGGAGAAAGTTGAAAAGCAGGACAAAGAGAAACCTGAGAAAG
ACAAGGAAATTAGTCCTAGTGTTACCAAGAAAAATACCAACAAGAAAACC
AAACCAAAGTCTGACATTCTGAAAGATCCTCCTAGTGAAGCAAACAGCAT
ACAGTCTGCAAATGCTACAACAAAGACCAGCGAAACAAATCACACCTCAA
GGCCCCGGCTGAAAAACGTGGACAGGAGCACTGCACAGCAGTTGGCAGTA
ACTGTGGGCAACGTCACCGTCATTATCACAGACTTTAAGGAAAAGACTCG
CTCCTCATCGACATCCTCATCCACAGTGACCTCCAGTGCAGGGTCAGAAC
AGCAGAACCAGAGCAGCTCGGGGTCAGAGAGCACAGACAAGGGCTCCTCC
CGTTCCTCCACGCCAAAGGGCGACATGTCAGCAGTCAATGATGAATCTTT
CTGAAATTGCACATGGAATTGTGAAAACTATGAATCAGGGTATGAAATTC
AAAACCTCCACCTGCCCATGCTGCTTGCATCCCTGGAGAATCTTCTGTGG
ACATCGACCTCTTAGTGATGCTGCCAGGATAATTTCTGCTTGCCATGGGC
ATCTGGCCACCAAGGAATTTCGCACCCTGACGATTACTCTTGACACTTTT
ATGTATTCCATTGTTTTATATGATTTTCCTAACAATCATTTATAATTGGA
TGTGCTCCTGAATCTACTTTTAATAAAAAAAAAAAA
MLL

SEQ ID NO:85

>gi|5174568|ref|NM_005933.1|Homo sapiens myeloid/
lymphoid or mixed-lineage leukemia (trithorax
homolog, Drosophila) (MLL), mRNA
ATGGCGCACAGCTGTCGGTGGCGCTTCCCCGCCCGACCCGGGACCACCGG
GGGCGGCGGCGGCGGGGGGCGCCGGGGCCTAGGGGGCGNCCCGCGGCAAC
GCGTCCCGGCCCTGCTGCTTCCCCCCGGGCCCCCGGTCGGCGGTGGCGGC
CCCGGGGCGCCCCCCTCCCCCCCGGCTGTGGCGGCCGCGGCGGCGGCGGC
GGGAAGCAGCGGGGCTGGGGTTCCAGGGGGAGCGGCCGCCGCCTCAGCAG
CCTCCTCGTCGTCCGCCTCGTCTTCGTCTTCGTCATCGTCCTCAGCCTCT
TCAGGGCCGGCCCTGCTCCGGGTGGGCCCGGGCTTCGACGCGGCGCTGCA
GGTCTCGGCCGCCATCGGCACCAACCTGCGCCGGTTCCGGGCCGTGTTTG
GGGAGAGCGGCGGGGGAGGCGGCAGCGGAGAGGATGAGCAATTCTTAGGT
TTTGGCTCAGATGAAGAAGTCAGAGTGCGAAGTCCCACAAGGTCTCCTTC
AGTTAAAACTAGTCCTCGAAAACCTCGTGGGAGACCTAGAAGTGGCTCTG
ACCGAAATTCAGCTATCCTCTCAGATCCATCTGTGTTTTCCCCTCTAAAT
AAATCAGAGACCAAATCTGGAGATAAGATCAAGAAGAAAGATTCTAAAAG
TATAGAAAAGAAGAGAGGAAGACCTCCCACCTTCCCTGGAGTAAAAATCA
AAATAACACATGGAAAGGACATTTCAGAGTTACCAAAGGGAAACAAAGAA
GATAGCCTGAAAAAAATTAAAAGGACACCTTCTGCTACGTTTCAGCAAGC
CACAAAGATTAAAAAATTAAGAGCAGGTAAACTCTCTCCTCTCAAGTCTA
AGTTTAAGACAGGGAAGCTTCAAATAGGAAGGAAGGGGGTACAAATTGTA
CGACGGAGAGGAAGGCCTCCATCAACAGAAAGGATAAAGACCCCTTCGGT
CTCCTCATTAATTCTGAACTGGAAAAGCCCCAGAAAGTCCGGAAAGACAA
GGAAGGAACACCTCCACTTACAAAAGAAGATAAGACAGTTGTCAGACAAA
GCCCTCGAAGGATTAAGCCAGTTAGGATTATTCCTTCTTCAAAAAGGACA
GATGCAACCATTGCTAAGCAACTCTTACAGAGGGCAAAAAAAGGGGGCTC
AAAAGAAAATTGAAAAAGAAGCAGCTCAGCTGCAGGGAAGAAAGGTGAAG
ACACAGGTCAAAAATATTCGACAGTTCATCATGCCTGTTGTCAGTGCTAT
CTCCTCGCGGATCATTAAGACCCCTCGGCGGTTTATAGAGGATGAGGATT
ATGACCCTCCAATTAAAATTGCCCGATTAGAGTCTACACCGAATAGTAGA
TTCAGTGCCCCGTCCTGTGGATCTTCTGAAAAATCAAGTGCAGCTTCTCA
GCACTCCTCTCAAATGTCTTCAGACTCCTCTCGATCTAGTAGCCCCAGTG
TTGATACCTCCACAGACTCTCAGGCTTCTGAGGAGATTCAGGTACTTCCT
GAGGAGCGGAGCGATACCCCTGAAGTTCATCCTCCACTGCCCATTTCCCA
GTCCCCAGAAAATGAGAGTAATGATAGGAGAAGCAGAAGGTATTCAGTGT
CGGAGAGAAGTTTTGGATCTAGAACGACGAAAAAATTATCAACTCTACAA
AGTGCCCCCCAGCAGCAGACCTCCTCGTCTCCACCTCCACCTCTGCTGAC
TCCACCGCCACCACTGCAGCCAGCCTCCAGTATCTCTGACCACACACCTT
GGCTTATGCCTCCAACAATCCCCTTAGCATCACCATTTTTGCCTGCTTCC
ACTGCTGGTATGCAAGGGAAGCGAAAATCTATTTTGCGAGAACCGACATT
TAGGTGGACTTCTTTAAAGCATTCTAGGTCAGAGCCACAATACTTTTCCT
CAGCAAAGTATGCCAAAGAAGGTCTTATTCGCAAACCAATATTTGATAAT
TTCCGACCCCCTCCACTAACTCCCGAGGACGTTGGCTTTGCATCTGGTTT
TTCTGCATCTGGTACCGCTGCTTCAGCCCGATTGTTTTCGCCACTCCATT
CTGGAACAAGGTTTGATATGCACAAAAGGAGCCCTCTTCTGAGAGCTCCA
AGATTTACTCCAAGTGAGGCTCACTCTAGAATATTTGAGTCTGTAACCTT
GCCTAGTAATCGAACTTCTGCTGGAACATCTTCTTCAGGAGTATCCAATA
GAAAAAGGAAAAGAAAAGTGTTTAGTCCTATTCGATCTGAACCAAGATCT
CCTTCTCACTCCATGAGGACAAGAAGTGGAAGGCTTAGTAGTTCTGAGCT
CTCACCTCTCACCCCCCCGTCTTCTGTCTCTTCCTCGTTAAGCATTTCTG
TTAGTCCTCTTGCCACTAGTGCCTTAAACCCAACTTTTACTTTTCCTTCT
CATTCCCTGACTCAGTCTGGGGAATCTGCAGAGAAAAATCAGAGACCAAG
GAAGCAGACTAGTGCTCCGGCAGAGCCATTTTCATCAAGTAGTCCTACTC
CTCTCTTCCCTTGGTTTACCCCAGGCTCTCAGACTGAAAGAGGGAGAAAT
AAAGACAAGGCCCCCGAGGAGCTGTCGAAAGATCGAGATGCTGACAAGAG
CGTGGAGAAGGACAAGAGTAGAGAGAGAGACCGGGAGAGAGAAAAGGAGA
ATAAGCGGGAGTCAAGGAAAGAGAAAAGGAAAAAGGGATCAGAAATTCAG
AGTAGTTCTGCTTTGTATCCTGTGGGTAGGGTTTCCAAAGAGAAGGTTGT
TGGTGAAGATGTTGCCACTTCATCTTCTGCCAAAAAAGCAACAGGGCGGA
AGAAGTCTTCATCACATGATTCTGGGACTGATATTACTTCTGTGACTCTT
GGGGATACAACAGCTGTCAAAACCAAAATACTTATAAAGAAAGGGAGAGG
AAATCTGGAAAAAACCAACTTGGACCTCGGCCCAACTGCCCCATCCCTGG
AGAAGGAGAAAACCCTCTGCCTTTCCACTCCTTCATCTAGCACTGTTAAA
CATTCCACTTCCTCCATAGGCTCCATGTTGGCTCAGGCAGACAAGCTTCC
AATGACTGACAAGAGGGTTGCCAGCCTCCTAAAAAAGGCCAAAGCTCAGC
TCTGCAAGATTGAGAAGAGTAAGAGTCTTAAACAAACCGACCAGCCCAAA
GCACAGGGTCAAGAAAGTGACTCATCAGAGACCTCTGTGCGAGGACCCCG
GATTAAACATGTCTGCAGAAGAGCAGCTGTTGCCCTTGGCCGAAAACGAG
CTGTGTTTCCTGATGACATGCCCACCCTGAGTGCCTTACCATGGGAAGAA
CGAGAAAAGATTTTGTCTTCCATGGGGAATGATGACAAGTCATCAATTGC
TGGCTCAGAAGATGCTGAACCTCTTGCTCCACCCATCAAACCAATTAAAC
CTGTCACTAGAAACAAGGCACCCCAGGAACCTCCAGTAAAGAAAGGACGT
CGATCGAGGCGGTGTGGGCAGTGTCCCGGCTGCCAGGTGCCTGAGGACTG
TGGTGTTTGTACTAATTGCTTAGATAAGCCCAAGTTTGGTGGTCGCAATA
TAAAGAAGCAGTGCTGCAAGATGAGAAAATGTCAGAATCTACAATGGATG
CCTTCCAAAGCCTACCTGCAGAAGCAAGCTAAAGCTGTGAAAAAGAAAGA
GAAAAAGTCTAAGACCAGTGAAAAGAAAGACAGCAAAGAGAGCAGTGTTG
TGAAGAACGTGGTGGACTCTAGTCAGAAACCTACCCCATCAGCAAGAGAG
GATCCTGCCCCAAAGAAAAGCAGTAGTGAGCCTCCTCCACGAAAGCCCGT
CGAGGAAAAGAGTGAAGAAGGGAATGTCTCGGCCCCTGGGCCTGAATCCA
AACAGGCCACCACTCCAGCTTCCAGGAAGTCAAGCAAGCAGGTCTCCCAG
CCAGCACTGGTCATCCCGCCTCAGCCACCTACTACAGGACCGCCAAGAAA
AGAAGTTCCCAAAACCACTCCTAGTGAGCCCAAGAAAAAGCAGCCTCCAC
CACCAGAATCAGGTCCAGAGCAGAGCAAACAGAAAAAAGTGGCTCCCCGC
CCAAGTATCCCTGTAAAACAAAAACCAAAAGAAAAGGAAAAACCACCTCC
GGTCAATAAGCAGGAGAATGCAGGCACTTTGAACATCCTCAGCACTCTCT
CCAATGGCAATAGTTCTAAGCAAAAAATTCCAGCAGATGGAGTCCACAGG
ATCAGAGTGGACTTTAAGGAGGATTGTGAAGCAGAAAATGTGTGGGAGAT
GGGAGGCTTAGGAATCTTGACTTCTGTTCCTATAACACCCAGGGTGGTTT
GCTTTCTCTGTGCCAGTAGTGGGCATGTAGAGTTTGTGTATTGCCAAGTC
TGTTGTGAGCCCTTCCACAAGTTTTGTTTAGAGGAGAACGAGCGCCCTCT
GGAGGACCAGCTGGAAAATTGGTGTTGTCGTCGTTGCAAATTCTGTCACG
TTTGTGGAAGGCAACATCAGGCTACAAAGCAGCTGCTGGAGTGTAATAAG
TGCCGAAACAGCTATCACCCTGAGTGCCTGGGACCAAACTACCCCACCAA
ACCCACAAAGAAGAAGAAAGTCTGGATCTGTACCAAGTGTGTTCGCTGTA
AGAGCTGTGGATCCACAACTCCAGGCAAAGGGTGGGATGCACAGTGGTCT
CATGATTTCTCACTGTGTCATGATTGCGCCAAGCTCTTTGCTAAAGGAAA
CTTCTGCCCTCTCTGTGACAAATGTTATGATGATGATGACTATGAGAGTA
AGATGATGCAATGTGGAAAGTGTGATCGCTGGGTCCATTCCAAATGTGAG
AATCTTTCAGATGAGATGTATGAGATTCTATCTAATCTGCCAGAAAGTGT
GGCCTACACTTGTGTGAACTGTACTGAGCGGCACCCTGCAGAGTGGCGAC
TGGCCCTTGAAAAAGAGCTGCAGATTTCTCTGAAGCAAGTTCTGACAGCT
TTGTTGAATTCTCGGACTACCAGCCATTTGCTACGCTACCGGCAGGCTGC
CAAGCCTCCAGACTTAAATCCCGAGACAGAGGAGAGTATACCTTCCCGCA
GCTCCCCCGAAGGACCTGATCCACCAGTTCTTACTGAGGTCAGCAAACAG
GATGATCAGCAGCCTTTAGATCTAGAAGGAGTCAAGAGGAAGATGGACCA
AGGGAATTACACATCTGTGTTGGAGTTCAGTGATGATATTGTGAAGATCA
TTCAAGCAGCCATTAATTCAGATGGAGGACAGCCAGAAATTAAAAAAGCC
AACAGCATGGTCAAGTCCTTCTTCATTCGGCAAATGGAACGTGTTTTTCC
ATGGTTCAGTGTCAAAAAGTCCAGGTTTTGGGAGCCAAATAAAGTATCAA
GCAACAGTGGGATGTTACCAAACGCAGTGCTTCCACCTTCACTTGACCAT
AATTATGCTCAGTGGCAGGAGCGAGAGGAAAACAGCCACACTGAGCAGCC
TCCTTTAATGAAGAAAATCATTCCAGCTCCCAAACCCAAAGGTCCTGGAG
AACCAGACTCACCAACTCCTCTGCATCCTCCTACACCACCAATTTTGAGT
ACTGATAGGAGTCGAGAAGACAGTCCAGAGCTGAACCCACCCCCAGGCAT
AGAAGACAATAGACAGTGTGCGTTATGTTTGACTTATGGTGATGACAGTG
CTAATGATGCTGGTCGTTTACTATATATTGGCCAAAATGAGTGGACACAT
GTAAATTGTGCTTTGTGGTCAGCGGAAGTGTTTGAAGATGATGACGGATC
ACTAAAGAATGTGCATATGGCTGTGATCAGGGGCAAGCAGCTGAGATGTG
AATTCTGCCAAAAGCCAGGAGCCACCGTGGGTTGCTGTCTCACATCCTGC
ACCAGCAACTATCACTTCATGTGTTCCCGAGCCAAGAACTGTGTCTTTCT
GGATGATAAAAAAGTATATTGCCAACGACATCGGGATTTGATCAAAGGCG
AAGTGGTTCCTGAGAATGGATTTGAAGTTTTCAGAAGAGTGTTTGTGGAC
TTTGAAGGAATCAGCTTGAGAAGGAAGTTTCTCAATGGCTTGGAACCAGA
AAATATCCACATGATGATTGGGTCTATGACAATCGACTGCTTAGGAATTC
TAAATGATCTCTCCGACTGTGAAGATAAGCTCTTTCCTATTGGATATCAG
TGTTCCAGGGTATACTGGAGCACCACAGATGCTCGCAAGCGCTGTGTATA
TACATGCAAGATAGTGGAGTGCCGTCCTCCAGTCGTAGAGCCGGATATCA
ACAGCACTGTTGAACATGATGAAAACAGGACCATTGCCCATAGTCCAACA
TCTTTTACAGAAAGTTCATCAAAAGAGAGTCAAAACACAGCTGAAATTAT
AAGTCCTCCATCACCAGACCGACCTCCTCATTCACAAACCTCTGGCTCCT
GTTATTATCATGTCATCTCAAAGGTCCCCAGGATTCGAACACCCAGTTAT
TCTCCAACACAGAGATCCCCTGGCTGTCGACCGTTGCCTTCTGCAGGAAG
TCCTACCCCAACCACTCATGAAATAGTCACAGTAGGTGATCCTTTACTCT
CCTCTGGACTTCGAAGCATTGGCTCCAGGCGTCACAGTACCTCTTCCTTA
TCACCCCAGCGGTCCAAACTCCGGATAATGTCTCCAATGAGAACTGGGAA
TACTTACTCTAGGAATAATGTTTCCTCAGTCTCCACCACCGGGACCGCTA
CTGATCTTGAATCAAGTGCCAAAGTAGTTGATCATGTCTTAGGGCCACTG
AATTCAAGTACTAGTTTAGGGCAAAACACTTCCACCTCTTCAAATTTGCA
AAGGACAGTGGTTACTGTAGGCAATAAAAACAGTCACTTGGATGGATCTT
CATCTTCAGAAATGAAGCAGTCCAGTGCTTCAGACTTGGTGTCCAAGAGC
TCCTCTTTAAAGGGAGAGAAGACCAAAGTGCTGAGTTCCAAGAGCTCAGA
GGGATCTGCACATAATGTGGCTTACCCTGGAATTCCTAAACTGGCCCCAC
AGGTTCATAACACAACATCTAGAGAACTGAATGTTAGTAAAATCGGCTCC
TTTGCTGAACCCTCTTCAGTGTCGTTTTCTTCTAAAGAGGCCCTCTCCTT
CCCACACCTCCATTTGAGAGGGCAAAGGAATGATCGAGACCAACACACAG
ATTCTACCCAATCAGCAAACTCCTCTCCAGATGAAGATACTGAAGTCAAA
ACCTTGAAGCTATCTGGAATGAGCAACAGATCATCCATTATCAACGAACA
TATGGGATCTAGTTCCAGAGATAGGAGACAGAAAGGGAAAAAATCCTGTA
AAGAAACTTTCAAAGAAAAGCATTCCAGTAAATCTTTTTTGGAACCTGGT
CAGGTGACAACTGGTGAGGAAGGAAACTTGAAGCCAGAGTTTATGGATGA
GGTTTTGACTCCTGAGTATATGGGCCAACGACCATGTAACAATGTTTCTT
CTGATAAGATTGGTGATAAAGGCCTTTCTATGCCAGGAGTCCCCAAAGCT
CCACCCATGCAAGTAGAAGGATCTGCCAAGGAATTACAGGCACCACGGAA
ACGCACAGTCAAAGTGACACTGACACCTCTAAAAATGGAAAATGAGAGTC
AATCCAAAAATGCCCTGAAAGAAAGTAGTCCTGCTTCCCCTTTGCAAATA
GAGTCAACATCTCCCACAGAACCAATTTCAGCCTCTGAAAATCCAGGAGA
TGGTCCAGTGGCCCAACCAAGCCCCAATAATACCTCATGCCAGGATTCTC
AAAGTAACAACTATCAGAATCTTCCAGTACAGGACAGAAACCTAATGCTT
CCAGATGGCCCCAAACCTCAGGAGGATGGCTCTTTTAAAAGGAGGTATCC
CCGTCGCAGTGCCCGTGCACGTTCTAACATGTTTTTTGGGCTTACCCCAC
TCTATGGAGTAAGATCCTATGGTGAAGAAGACATTCCATTCTACAGCAGC
TCAACTGGGAAGAAGCGAGGCAAGAGATCAGCTGAAGGACAGGTGGATGG
GGCCGATGACTTAAGCACTTCAGATGAAGACGACTTATACTATTACAACT
TCACTAGAACAGTGATTTCTTCAGGTGGAGAGGAACGACTGGCATCCCAT
AATTTATTTCGGGAGGAGGAACAGTGTGATCTTCCAAAAATCTCACAGTT
GGATGGTGTTGATGATGGGACAGAGAGTGATACTAGTGTCACAGCCACAA
CAAGGAAAAGCAGCCAGATTCCAAAAAGAAATGGTAAAGAAAATGGAACA
GAGAACTTAAAGATTGATAGACCTGAAGATGCTGGGGAGAAAGAACATGT
CACTAAGAGTTCTGTTGGCCACAAAAATGAGCCAAAGATGGATAACTGCC
ATTCTGTAAGCAGAGTTAAAACACAGGGACAAGATTCCTTGGAAGCTCAG
CTCAGCTCATTGGAGTCAAGCCGCAGAGTCCACACAAGTACCCCCTCCGA
CAAAAATTTACTGGACACCTATAATACTGAGCTCCTGAAATCAGATTCAG
ACAATAACAACAGTGATGACTGTGGGAATATCCTGCCTTCAGACATTATG
GACTTTGTACTAAAGAATACTCCATCCATGCAGGCTTTGGGTGAGAGCCC
AGAGTCATCTTCATCAGAACTCCTGAATCTTGGTGAAGGATTGGGTCTTG
ACAGTAATCGTGAAAAAGACATGGGTCTTTTTGAAGTATTTTCTCAGCAG
CTGCCTACAACAGAACCTGTGGATAGTAGTGTCTCTTCCTCTATCTCAGC
AGAGGAACAGTTTGAGTTGCCTCTAGAGCTACCATCTGATCTGTCTGTCT
TGACCACCCGGAGTCCCACTGTCCCCAGCCAGAATCCCAGTAGACTAGCT
GTTATCTCAGACTCAGGGGAGAAGAGAGTAACCATCACAGAAAAATCTGT
AGCCTCCTCTGAAAGTGACCCAGCACTGCTGAGCCCAGGAGTAGATCCAA
CTCCTGAAGGCCACATGACTCCTGATCATTTTATCCAAGGACACATGGAT
GCAGACCACATCTCTAGCCCTCCTTGTGGTTCAGTAGAGCAAGGTCATGG
CAACAATCAGGATTTAACTAGGAACAGTAGCACCCCTGGCCTTCAGGTAC
CTGTTTCCCCAACTGTTCCCATCCAGAACCAGAAGTATGTGCCCAATTCT
ACTGATAGTCCTGGCCCGTCTCAGATTTCCAATGCAGCTGTCCAGACCAC
TCCACCCCACCTGAAGCCAGCCACTGAGAAACTCATAGTTGTTAACCAGA
ACATGCAGCCACTTTATGTTCTCCAAACTCTTCCAAATGGAGTGACCCAA
AAAATCCAATTGACCTCTTCTGTTAGTTCTACACCCAGTGTGATGGAGAC
AAATACTTCAGTATTGGGACCCATGGGAGGTGGTCTCACCCTTACCACAG
GACTAAATCCAAGCTTGCCAACTTCTCAATCTTTGTTCCCTTCTGCTAGC
AAAGGATTGCTACCCATGTCTCATCACCAGCACTTACATTCCTTCCCTGC
AGCTACTCAAAGTAGTTTCCCACCAAACATCAGCAATCCTCCTTCAGGCC
TGCTTATTGGGGTTCAGCCTCCTCCGGATCCCCAACTTTTGGTTTCAGAA
TCCAGCCAGAGGACAGACCTCAGTACCACAGTAGCCACTCCATCCTCTGG
ACTCAAGAAAAGACCCATATCTCGTCTACAGACCCGAAAGAATAAAAAAC
TTGCTCCCTCTAGTACCCCTTCAAACATTGCCCCTTCTGATGTGGTTTCT
AATATGACATTGATTAACTTCACACCCTCCCAGCTTCCTAATCATCCAAG
TCTGTTAGATTTGGGGTCACTTAATACTTCATCTCACCGAACTGTCCCCA
ACATCATAAAAAGATCTAAATCTAGCATCATGTATTTTGAACCGGCACCC
CTGTTACCACAGAGTGTGGGAGGAACTGCTGCCACAGCGGCAGGCACATC
AACAATAAGCCAGGATACTAGCCACCTCACATCAGGGTCTGTGTCTGGCT
TGGCATCCAGTTCCTCTGTCTTGAATGTTGTATCCATGCAAACTACCACA
ACCCCTACAAGTAGTGCGTCAGTTCCAGGACACGTCACCTTAACCAACCC
AAGGTTGCTTGGTACCCCAGATATTGGCTCAATAAGCAATCTTTTAATCA
AAGCTAGCCAGCAGAGCCTGGGGATTCAGGACCAGCCTGTGGCTTTACCG
CCAAGTTCAGGAATGTTTCCACAACTGGGGACATCACAGACCCCCTCTAC
TGCTGCAATAACAGCGGCATCTAGCATCTGTGTGCTCCCCTCCACTCAGA
CTACGGGCATAACAGCCGCTTCACCTTCTGGGGAAGCAGACGAACACTAT
CAGCTTCAGCATGTGAACCAGCTCCTTGCCAGCAAAACTGGGATTCATTC
TTCCCAGCGTGATCTTGATTCTGCTTCAGGGCCCCAGGTATCCAACTTTA
CCCAGACGGTAGACGCTCCTAATAGCATGGGACTGGAGCAGAACAAGGCT
TTATCCTCAGCTGTGCAAGCCAGCCCCACCTCTCCTGGGGGTTCTCCATC
CTCTCCATCTTCTGGACAGCGGTCAGCAAGCCCTTCAGTGCCGGGTCCCA
CTAAACCCAAACCAAAAACCAAACGGTTTCAGCTGCCTCTAGACAAAGGG
AATGGCAAGAAGCACAAAGTTTCCCATTTGCGGACCAGTTCTTCTGAAGC
ACACATTCCAGACCAAGAAACGACATCCCTGACCTCAGGCACAGGGACTC
CAGGAGCAGAGGCTGAGCAGCAGGATACAGCTAGCGTGGAGCAGTCCTCC
CAGAAGGAGTGTGGGCAACCTGCAGGGCAAGTCGCTGTTCTTCCGGAAGT
TCAGGTGACCCAAAATCCAGCAAATGAACAAGAAAGTGCAGAACCTAAAA
CAGTGGAAGAAGAGGAAAGTAATTTCAGCTCCCCACTGATGCTTTGGCTT
CAGCAAGAACAAAAGCGGAAGGAAAGCATTACTGAGAAAAAACCCAAGAA
AGGACTTGTTTTTGAAATTTCCAGTGATGATGGCTTTCAGATCTGTGCAG
AAAGTATTGAAGATGCCTGGAAGTCATTGACAGATAAAGTCCAGGAAGCT
CGATCAAATGCCCGCCTAAAGCAGCTCTCATTTGCAGGTGTTAACGGTTT
GAGGATGCTGGGGATTCTCCATGATGCAGTTGTGTTCCTCATTGAGCAGC
TGTCTGGTGCCAAGCACTGTCGAAATTACAAATTCCGTTTCCACAAGCCA
GAGGAGGCCAATGAACCCCCCTTGAACCCTCACGGCTCAGCCAGGGCTGA
AGTCCACCTCAGGAAGTCAGCATTTGACATGTTTAACTTCCTGGCTTCTA
AACATCGTCAGCCTCCTGAATACAACCCCAATGATGAAGAAGAGGAGGAG
GTACAGCTGAAGTCAGCTCGGAGGGCAACTAGCATGGATCTGCCAATGCC
CATGCGCTTCCGGCACTTAAAAAAGACTTCTAAGGAGGCAGTTGGTGTCT
ACAGGTCTCCCATCCATGGCCGGGGTCTTTTCTGTAAGAGAAACATTGAT
GCAGGTGAGATGGTGATTGAGTATGCCGGCAACGTCATCCGCTCCATCCA
GACTGACAAGCGGGAAAAGTATTACGACAGCAAGGGCATTGGTTGCTATA
TGTTCCGAATTGATGACTCAGAGGTAGTGGATGCCACCATGCATGGAAAT
CGTGCACGCTTCATCAATCACTCGTGTGAGCCTAACTGCTATTCTCGGGT
CATCAATATTGATGGGCAGAAGCACATTGTCATCTTTGCCATGCGTAAGA
TCTACCGAGGAGAGGAACTCACTTACGACTATAAGTTCCCCATTGAGGAT
GCCAGCAACAAGCTGCCCTGCAACTGTGGCGCCAAGAAATGCCGGAAGTT
CCTAAACTAA
MLLT1

SEQ ID NO:86

>gi|5174570|ref|NM_005934.1|Homo sapiens myeloid/
lymphoid or mixed-lineage leukemia (trithorax
homolog, Drosophila); translocated to, 1 (MLLT1),
mRNA
ATGGACAATCAGTGCACCGTCCAGGTGAGGTTAGAGCTGGGGCATCGCGC
CCAACTGCGCAAGAAGCCCACCACGGAGGGGTTCACTCACGACTGGATGG
TGTTTGTCCGCGGCCCCGAGCAATGTGACATCCAGCACTTCGTGGAGAAG
GTGGTCTTCTGGCTGCACGACAGCTTCCCCAAGCCCAGACGCGTGTGCAA
GGAGCCCCCCTACAAAGTAGAGGAGTCGGGGTACGCTGGCTTCATCATGC
CCATCGAGGTGCACTTCAAAAACAAGGAGGAGCCGAGGAAGGTCTGCTTC
ACCTACGACCTGTTCCTGAACCTGGAAGGCAACCCGCCCGTGAACCACCT
GCGCTGCGAGAAGCTCACCTTCAACAACCCCACCACGGAGTTCCGGTACA
AGCTCCTGCGGGCCGGCGGGGTGATGGTAATGCCCGAAGGAGCAGACACG
GTGTCCAGGCCCAGTCCCGACTACCCCATGTTACCCACAATTCCACTCTC
TGCCTTCTCTGACCCCAAGAAGACCAAACCATCCCACGGCTCCAAGGACG
CCAACAAGGAGAGCAGCAAGACCTCCAAGCCACACAAGGTGACCAAGGAG
CACCGGGAGCGCCCCCGCAAAGACTCCGAGAGCAAGAGCTCCTCCAAGGA
GCTGGAGCGTGAGCAGGCCAAAAGCTCCAAGGACACCTCGCGGAAGCTGG
GCGAGGGCCGGCTGCCCAAGGAGGAGAAGGCGCCACCGCCCAAGGCTGCC
TTCAAGGAACCCAAGATGGCCCTGAAAGAGACCAAGCTGGAAAGCACGTC
CCCCAACCCTGGGCCCCCACCCCCACCCCCACCCCCACCCCGGGCTTCCA
GCAAGCGGCCGGCCACCGCCGACTCGCCAAAGCCCAGCGCCAAGAAGCAG
AAGAAGAGCAGCTCGAAGGGGTCCCGGAGTGCTCCAGGCACCTCGCCCCG
CACCTCCTCCTCCTCCTCCTTCTCGGACAAGAAGCCGGCCAAGGACAAGA
GCAGCACCAGAGGGGAGAAGGTGAAGGCCGAGAGTGAGCCCCGGGAGGCC
AAAAAGGCCCTGGAGGTGGAGGAGTCCAACTCAGAGGACGAGGCCTCCTT
CAAGTCCGAGTCTGCCCAGTCAAGCCCGTCCAACTCCAGCTCCAGCTCAG
ACTCCAGCTCAGACTCAGACTTCGAGCCATCCCAGAACCACAGCCAAGGA
CCCCTGCGCTCCATGGTGGAGGACCTGCAGTCCGAGGAGTCCGACGAGGA
CGACTCTTCGTCAGGCGAGGAGGCTGCCGGCAAGACCAACCCGGGGAGGG
ACTCCAGGTTGAGCTTCAGCGACAGCGAGAGTGACAACAGCGCCGACTCC
TCCCTGCCCAGCCGTGAGCCCCCACCCCCCCAGAAGCCACCCCCGCCCAA
CAGCAAGGTGTCAGGCCGGAGGAGCCCCGAGTCCTGCAGCAAGCCTGAGA
AGATCCTCAAGAAGGGCACCTACGACAAGGCCTACACGGATGAGCTGGTG
GAGCTACACCGGAGGCTGATGGCGCTGCGGGAGCGCAACGTGCTGCAGCA
GATTGTGAATCTGATCGAGGAGACTGGCCACTTCAATGTCACCAACACCA
CCTTCGACTTCGACCTCTTCTCCCTGGACGAGACCACCGTGCGCAAACTG
CAGAGCTGCCTGGAGGCCGTGGCCACATGA
MLLT7

SEQ ID NO:87

>gi|5174578|ref|NM_005938.1|Homo sapiens myeloid/
lymphoid or mixed-lineage leukemia (trithorax
homolog, Drosophila); translocated to, 7 (MLLT7),
mRNA
GGGACAGCTTAGGGACTATCGTCCTGGGACTAGGGGGAAGTTCGCGACTT
TCTGAAGACTGGCAGGAATGTGCCTCCTGGCCCTCGATGCTTCCCCCCTG
AGGGGAGGCATCGTGAGGGACTGTGGCAGGCTTCACTGAACGCTGAGCCG
GGGAGGTCCAACTCCACGTATGGATCCGGGGAATGAGAATTCAGCCACAG
AAGGCCGCCGCGATCATAGACCTAGATCCCGACTTCGAACCCCAGAGCCG
TCCCCGCTCCTGCACCTGGCCCCTTCCCCGACCAGAGATCGCTAACCAGC
CGTCCGAGCCGCCCGAGGTGGAGCCAGATCTGGGGGAAAAGGTACACACG
GAGGGGCGCTCAGAGCCGATCCTGTTGCCCTCTCGGCTCTCAGAGCCGGC
CGGGGGCCCCCAGCCCGGAATCCTGGGGGCTGTAACAGGTCCTCGGAAGG
GAGGCTCCCGCCGGAATGCCTGGGGAAATCAGTCATATGCAGAATTCATC
AGCCAGGCCATTGAAAGCGCCCCGGAGAAGCGACTGACACTTGCCCAGAT
TTACGAGTGGATGGTCCGTACTGTACCCTACTTCAAGGACAAGGGTGACA
GCAACAGCTCAGCAGGATGGAAGAACTCGATCCGCCACAACCTGTCCCTG
CACAGCAAGTTCATCAAGGTTCACAACGAGGCCACCGGCAAAAGCTCTTG
GTGGATGCTGAACCCTGAGGGAGGCAAGAGCGGCAAAGCCCCCCGCCGCC
GGGCCGCCTCCATGGATAGCAGCAGCAAGCTGCTCCGGGGCCGCAGTAAA
GCCCCCAAGAAGAAACCATCTGTGCTGCCAGCTCCACCCGAAGGTGCCAC
TCCAACGAGCCCTGTCGGCCACTTTGCCAAGTGGTCAGGCAGCCCTTGCT
CTCGAAACCGTGAAGAAGCCGATATGTGGACCACCTTCCGTCCACGAAGC
AGTTCAAATGCCAGCAGTGTCAGCACCCGGCTGTCCCCCTTGAGGCCAGA
GTCTGAGGTGCTGGCGGAGGAAATACCAGCTTCAGTCAGCAGTTATGCAG
GGGGTGTCCCTCCCACCCTCAATGAAGGTCTAGAGCTGTTAGATGGGCTC
AATCTCACCTCTTCCCATTCCCTGCTATCTCGGAGTGGTCTCTCTGGCTT
CTCTTTGCAGCATCCTGGGGTTACCGGCCCCTTACACACCTACAGCAGCT
CCCTTTTCAGCCCAGCAGAGGGGCCCCTGTCAGCAGGAGAAGGGTGCTTC
TCCAGCTCCCAGGCTCTGGAGGCCCTGCTCACCTCTGATACGCCACCACC
CCCTGCTGACGTCCTCATGACCCAGGTAGATCCCATTCTGTCCCAGGCTC
CGACTCTTCTGTTGCTGGGGGGGCTTCCTTCCTCCAGTAAGCTGGCCACG
GGCGTCGGCCTGTGTCCCAAGCCCCTAGAGGCTCGAGGCCCCAGCAGTCT
GGTTCCCACCCTTTCTATGATAGCACCACCTCCAGTCATGGCAAGTGCCC
CCATCCCCAAGGCTCTGGGGACTCCTGTGCTCACACCCCCTACTGAAGCT
GCAAGCCAAGACAGAATGCCTCAGGATCTAGATCTTGATATGTATATGGA
GAACCTGGAGTGTGACATGGATAACATCATCAGTGACCTCATGGATGAGG
GCGAGGGACTGGACTTCAACTTTGAGCCAGATCCCTGAGTCATGCCTGGA
AGCTTTGTCCCCTGCTTCAGATGTGGAGCCAGGCGTGTTCATATCTACTC
TTTACCCTTGAGCCCTCCCCAGGAATTTGGGACCCTGCTTTAGAGCTAGG
GTGGGGTCTGGTCACACACAGGTGTTGAAGAAATTATAAAGATAAAGCTG
CCCCATCTGGGGACGATATGGGGAGGGAGATGGGAGGGGAAAGGGGAGAG
GGTTTTTCTCACTGTGCCAATTAGGGGGTAAGGCCCCCTCTCAGGAGCCA
TCATCGGCTTTCCCCATTCCTACCCACTTAGGCTTTGTAGCAAGATGAGC
AATGCTGTTGGAAATGTGAAGTCACCAGTGGCCTTACCCCTGCCTTTGGG
AGCAGGATTTTTTTGTAGAGAGTCTTATCTGAGCTGAGCCAGGCTAGCTG
GAGCCTGGGATTTCTATGCAGTGGCCCCTTAGGCCAGTGATGTGCGGTGG
GTGGGCTGTTTAGGGGATCTGGAAGGGCCAAGGTCTGAGCACTGGAGTGG
CTCGCCAGGCCAAATCACCCTTAGAAGGCTGCAGATAACAGAAAGGCTTT
TTATAAACTTTTAAAGAAATATAAACACAAATATAGAGATTTTTTAACCA
TGGCAGGGTGCTAGTGGTGGGCAGAATGCTTTTTTTTCTTTCTGAAGGCT
TTGTGATAGTGACATGATACAAACACTACAGACAATAAATATTAGGAGAC
ACAGGGAAGTGGGGAGAGGTGGGGAGTAATAGTAAACACAGGGAAGAGCT
CCCCTACGGACCAGGTATAGAGAAAGGTCTATGCAGAAATAGGTTAGAGT
TTCCCTAACAAAAAAGCTAACCCAGGTCCCCTCATTCCTTCAACTTGTGC
CTGGGAGTGTGTGGTGTTAGGGTGCAGCCACACTCTTCTATGACCCAGCA
TGGGTTAGTGCTATGGTGGGAGAGTACATTGAAGGCCTGGAATTAGCTTG
GGGCCAGGGAAGGGACTGGGAGGGGAGAGAAGAGAAGGAGGGAAGGATTT
AGGATGGTAAAGTTAGGTACAGAGACCTCCCTGTTCAAGGCCCCTGACAG
CTGTCCCTGCCCTTCTTCCCCTTCCCTGACTGCAGGGGTTATGTGGAAGT
GTGTGTGGCAGCAGGCAGCGGGGAGGGGAGGAACAGGGAAGGGGGAGCTG
GGGAGCTTGGCTGAGGGTCTGGGAAATGAGCAGGGATGGGGGGGGATGTG
GATCAGGTTTACTAGCACCTGCCAGGGAGGCCATCTGGGGCTCCTTCTCC
ACCCCAGCCCCCAAAGCAGCCCTTCCCCCAGTGCCCTTTGCATCGTCCCC
TCCCCCACCCCTGCTGTGGGTTCCCATCATTTCCTGTGTCAGCGCCTGGC
CTACCCAGATTGTATCATGTGCTAGATTGGAGTGGGGAAGTGTGTCAAAT
CAATAAATGAATAAATTCAATAAATGCCTATAACCAGCAGAAAAAAAAAA
AAAAAAAAAAAAAAAAAAAAA
MLLT6

SEQ ID NO:88

>gi|5174576|ref|NM_005937.1|Homo sapiens myeloid/
lymphoid or mixed-lineage leukemia (trithorax
homolog, Drosophila); translocated to, 6 (MLLT6),
mRNA
ATGAAGGAGATGGTAGGAGGCTGCTGCGTATGTTCGGACGAGAGGGGCTG
GGCCGAGAACCCGCTGGTCTACTGCGATGGGCACGCGTGCAGCGTGGCCG
TCCACCAAGCTTGCTATGGCATCGTTCAGGTGCCAACGGGACCCTGGTTC
TGCCGGAAATGTGAATCTCAGGAGCGAGCAGCCAGGGTGAGGTGTGAGCT
GTGCCCACACAAAGACGGGGCATTGAAGAGGACTGATAATGGAGGCTGGG
CACACGTGGTGTGTGCCCTCTACATCCCCGAGGTGCAATTTGCCAACGTG
CTCACCATGGAGCCCATCGTGCTGCAGTACGTGCCTCATGATCGCTTCAA
CAAGACCTGTTACATCTGCGAGGAGACGGGCCGGGAGAGCAAGGCGGCCT
CGGGAGCCTGCATGACCTGTAACCGCCATGGATGTCGACAAGCTTTCCAC
GTCACCTGTGCCCAAATGGCAGGCTTGCTGTGTGAGGAAGAAGTGCTGGA
GGTGGACAACGTCAAGTACTGCGGCTACTGCAAATACCACTTCAGCAAGA
TGAAGACATCCCGGCACAGCAGCGGGGGAGGCGGAGGAGGCGCTGGAGGA
GGAGGTGGCAGCATGGGGGGAGGTGGCAGTGGTTTCATCTCTGGGAGGAG
AAGCCGGTCAGCCTCACCATCCACGCAGCAGGAGAAGCACCCCACCCACC
ACGAGAGGGGCCAGAAGAAGAGTCGAAAGGACAAAGAACGCCTTAAGCAG
AAGCACAAGAAGCGGCCTGAGTCGCCCCCCAGCATCCTCACCCCGCCCGT
GGTCCCCACTGCTGACAAGGTCTCCTCCTCGGCTTCCTCTTCCTCCCACC
ACGAGGCCAGCACGCAGGAGACCTCTGAGAGCAGCAGGGAGTCAAAGGGG
AAAAAGTCTTCCAGCCATAGCCTGAGTCATAAAGGGAAGAAACTGAGCAG
TGGGAAAGGTGTGAGCAGTTTTACCTCCGCCTCCTCTTCTTCCTCCTCCT
CTTCCTCCTCCTCTGGGGGGCCCTTCCAGCCTGCAGTCTCGTCCCTGCAG
AGCTCCCCTGACTTCTCTGCATTCCCCAAGCTGGAGCAGCCAGAGGAGGA
CAAGTACTCCAAGCCCACAGCCCCCGCCCCTTCAGCCCCTCCTTCTCCCT
CAGCTCCCGAGCCCCCCAAGGCTGACCTTTTTGAGCAGAAGGTGGTCTTC
TCTGGCTTTGGGCCCATCATGCGCTTCTCCACCACCACCTCCAGCTCAGG
CCGGGCCCGGGCGCCCTCCCCTGGGGACTATAAGTCTCCCCACGTCACGG
GGTCTGGGGCCTCGGCAGGCACCCACAAACGGATGCCCGCACTGAGTGCC
ACCCCTGTGCCTGCTGATGAGACCCCTGAGACAGGCCTGAAGGAGAAGAA
GCACAAAGCCAGCAAGAGGAGCCGCCATGGGCCAGGCCGTCCCAAGGGCA
GCCGGAACAAGGAGGGCACTGGGGGCCCAGCTGCCCCATCCTTGCCCAGT
GCCCAGCTGGCTGGCTTTACCGCCACTGCTGCCTCACCCTTCTCTGGAGG
TTCCCTGGTCAGCTCCGGCCTGGGAGGTCTGTCCTCCCGAACCTTTGGGC
CTTCTGGGAGCTTGCCCAGCTTGAGCCTGGAGTCCCCCTTACTAGGGGCA
GGCATCTACACCAGTAATAAGGACCCCATCTCCCACAGTGGCGGGATGCT
GCGGGCTGTCTGCAGCACCCCTCTCTCCTCCAGCCTCCTGGGGCCCCCAG
GGACCTCGGCCCTGCCCCGCCTCAGCCGCTCCCCGTTCACCAGCACCCTC
CCCTCCTCTTCTGCTTCTATCTCCACCACTCAGGTGTTTTCTCTGGCTGG
CTCTACCTTTAGCCTCCCTTCTACCCACATCTTTGGAACCCCCATGGGTG
CCGTTAATCCCCTCCTCTCCCAAGCTGAGAGCAGCCACACAGAGCCAGAC
CTGGAGGACTGCAGCTTCCGGTGTCGGGGGACCTCCCCTCAGGAGAGTCT
GTCTTCCATGTCCCCCATCAGCAGCCTCCCCGCACTCTTCGACCAGACAG
CCTCTGCACCCTGTGGGGGCGGCCAGTTAGACCCGGCGGCCCCAGGGACG
ACTAACATGGAGCAGCTTCTGGAGAAGCAGGGCGACGGGGAGGCCGGCGT
CAACATCGTGGAGATGCTGAAGGCGCTGCACGCGCTGCAGAAGGAGAACC
AGCGGCTGCAAGAGCAGATCCTGAGCCTGACGGCCAAAAAGGAGCGGCTG
CAGATTCTCAACGTGCAGCTCTCTGTGCCCTTCCCTGCCCTGCCTGCTGC
CCTGCCTGCCGCCAACGGCCCTGTCCCTGGGCCCTATGGCCTGCCTCCCC
AAGCCGGGAGCAGCGACTCCTTGAGCACCAGCAAGAGCCCTCCGGGAAAG
AGCAGCCTCGGCCTGGACAACTCGCTGTCCACTTCTTCTGAGGACCCACA
CTCAGGCTGCCCGAGCCGCAGCAGCTCGTCGCTGTCCTTCCACAGCACGC
CCCCACCGCTGCCCCTCCTCCAGCAGAGCCCTGCCACTCTGCCCCTGGCC
CTGCCTGGGGCCCCTGCCCCACTCCCGCCCCAGCCGCAGAACGGGTTGGG
CCGGGCACCCGGGGCAGCGGGGCTGGGGGCCATGCCCATGGCTGAGGGGC
TGTTGGGGGGGCTGGCAGGCAGTGGGGGCCTGCCCCTCAATGGGCTCCTT
GGGGGGTTGAATGGGGCCGCTGCCCCCAACCCCGCAAGCTTGAGCCAGGC
TGGCGGGGCCCCCACGCTGCAGCTGCCAGGCTGTCTCAACAGCCTTACAG
AGCAGCAGAGACATCTCCTTCAGCAGCAAGAGCAGCAGCTCCAGCAACTC
CAGCAGCTCCTGGCCTCCCCGCAGCTGACCCCGGAACACCAGACTGTTGT
CTACCAGATGATCCAGCAGATCCAGCAGAAACGGGAGCTGCAGCGTCTGC
AGATGGCTGGGGGCTCCCAGCTGCCCATGGCCAGCCTGCTGGCAGGAAGC
TCCACCCCGCTGCTGTCTGCGGGTACCCCTGGCCTGCTGCCCACAGCGTC
TGCTCCACCCCTGCTGCCCGCTGGAGCCCTAGTGGCTCCCTCGCTTGGCA
ACAACACAAGTCTCATGGCCGCAGCAGCTGCAGCTGCAGCAGTAGCAGCA
GCAGGCGGACCTCCAGTCCTCACTGCCCAGACCAACCCCTTCCTCAGCCT
GTCGGGAGCAGAGGGCAGTGGCGGTGGCCCCAAAGGAGGGACCGCTGACA
AAGGAGCCTCAGCCAACCAGGAAAAAGGCTAA
MLLT4

SEQ ID NO:89

>gi|5174574|ref|NM_005936.1|Homo sapiens myeloid/
lymphoid or mixed-lineage leukemia (trithorax
homolog, Drosophila); translocated to, 4 (MLLT4),
mRNA
ATGTCGGCGGGCGGCCGTGACGAGGAGCGGCGGAAGCTGGCCGACATCAT
CCACCACTGGAACGCCAACCGGCTGGACCTGTTCGAGATCAGCCAGCCGA
CCGAGGATTTGGAGTTCCATGGAGTGATGAGATTTTATTTTCAAGATAAA
GCTGCTGGAAACTTTGCAACAAAATGTATTCGGGTCTCTAGTACTGCCAC
CACTCAAGATGTAATCGAAACGCTCGCGGAGAAATTTCGACCTGATATGC
GAATGCTGTCCTCTCCCAAGTATTCACTCTATGAAGTGCATGTCAGCGGA
GAAAGAAGATTGGATATAGATGAGAAACCTCTAGTTGTACAACTGAATTG
GAACAAAGATGATCGGGAAGGCAGATTTGTTCTTAAGAATGAGAATGACG
CCATTCCTCCTAAGGCTCAAAGTAATGGACCTGAAAAGCAGGAAAAAGAA
GGGGTTATCCAGAACTTCAAGAGAACTCTCTCAAAGAAAGAAAAGAAGGA
AAAAAAGAAGAGAGAAAAAGAGGCATTGCGACAGGCATCTGATAAAGATG
ATAGACCTTTCCAAGGGGAGGATGTTGAAAATTCTCGACTGGCTGCTGAG
GTTTACAAAGACATGCCGGAAACCAGCTTTACTCGAACCATTTCTAATCC
TGAGGTGGTTATGAAACGACGGAGGCAGCAAAAATTGGAAAAGAGAATGC
AGGAATTTCGGAGCTCAGATGGGCGGCCTGATTCAGGTGGAACATTGAGA
ATTTATGCAGATAGTTTAAAACCAAATATTCCCTACAAGACAATCCTGCT
GTCTACTACAGATCCTGCAGACTTTGCTGTGGCTGAAGCTTTAGAGAAGT
ATGGTCTGGAAAAAGAAAACCCTAAGGATTACTGCATCGCCCGGGTTATG
CTTCCTCCTGGAGCCCAGCATTCTGATGAAAAGGGTGCTAAAGAAATTAT
TCTTGATGATGATGAGTGTCCTTTACAAATCTTCAGGGAATGGCCAAGTG
ACAAAGGGATTTTAGTCTTTCAGTTGAAGAGGAGGCCACCAGACCACATC
CCAAAGAAAACCAAGAAACACTTGGAAGGCAAGACACCCAAGGGAAAGGA
GAGAGCTGACGGGTCTGTCTATGGCTCCACCCTTCCTCCGGAGAAGCTGC
CCTATTTAGTAGAGTTAAGCCCAGATGGTTCTGACTCTAGAGATAAGCCA
AAGCTTTACCGCCTTCAGTTAAGTGTTACTGAAGTTGGGACAGAAAAGTT
GGATGACAACTCTATCCAGTTGTTTGGCCCAGGAATTCAGCCCCATCACT
GTGACCTTACCAACATGGATGGAGTGGTCACTGTGACGCCCAGAAGTATG
GACGCAGAAACCTACGTGGAAGGCCAGCGCATCTCAGAAACCACCATGCT
GCAGAGTGGCATGAAAGTGCAGTTTGGGGCGTCCCATGTATTTAAGTTTG
TGGACCCCAGTCAGGATCATGCTCTTGCAAAAAGATCTGTGGATGGAGGC
CTGATGGTTAAGGGCCCAAGACATAAACCTGGAATTGTTCAGGAGACAAC
TTTTGATTTGGGAGGAGATATTCATAGTGGGACAGCATTACCGACAAGCA
AGAGCACCACTAGGCTGGACAGCGACAGAGTGTCGTCTGCCTCTAGCACA
GCCGAGCGGGGAATGGTGAAGCCGATGATCAGAGTAGAACAGCAGCCAGA
TTATCGCAGGCAAGAAAGCAGAACACAGGATGCTTCTGGGCCTGAGCTGA
TACTACCTGCAAGCATTGAATTCAGGGAAAGTTCTGAAGATTCATTTTTG
TCTGCCATTATAAATTATACTAATAGCTCTACAGTCCACTTTAAGTTGTC
CCCTACATATGTATTATATATGGCATGCCGGTATGTATTGTCCAACCAGT
ACAGACCTGACATCAGCCCTACAGAGCGCACACATAAAGTCATTGCAGTC
GTCAACAAGATGGTGAGCATGATGGAGGGTGTCATCCAGAAACAGAAGAA
TATTGCAGGGGCACTTGCCTTCTGGATGGCAAATGCATCTGAACTTCTCA
ACTTCATTAAGCAAGACCGAGACCTTAGTCGGATCACACTGGATGCTCAA
GATGTTTTAGCACATTTGGTTCAAATGGCATTTAAATACTTGGTTCACTG
TCTTCAATCAGAACTTAATAATTACATGCCAGCCTTTCTAGATGACCCTG
AAGAGAACAGTCTGCAACGACCAAAAATAGATGATGTGCTGCACACGCTC
ACAGGAGCCATGTCCTTGCTACGACGCTGCAGAGTCAATGCCGCCCTGAC
CATCCAGCTCTTCTCTCAGCTCTTCCACTTCATCAATATGTGGCTGTTCA
ATAGATTGGTGACCGACCCAGATTCGGGGCTGTGCTCCCATTACTGGGGT
GCGATTATCCGTCAGCAGTTGGGCCATATTGAAGCCTGGGCTGAGAAGCA
GGGGCTGGAACTGGCTGCGGACTGTCATCTGAGCAGGATCGTGCAGGCAA
CGACTTTGCTTACCATGGATAAGTATGCACCTGATGACATTCCAAATATA
AACAGCACCTGCTTTAAGTTAAATTCATTACAACTTCAAGCCTTATTACA
GAACTATCACTGTGCACCTGATGAGCCTTTTATCCCAACGGATCTTATAG
AAAATGTAGTGACTGTGGCTGAAAACACTGCCGATGAGCTGGCCCGCAGT
GATGGAAGGGAAGTGCAGTTGGAGGAGGATCCTGATCTGCAGCTGCCGTT
TCTTTTGCCAGAAGATGGTTATTCTTGTGATGTTGTCAGAAACATTCCAA
ATGGTTTACAAGAATTTTTAGACCCTCTGTGCCAGAGAGGATTTTGCAGG
TTAATTCCTCACACACGTTCACCAGGTACTTGGACAATATATTTTGAAGG
TGCAGATTATGAAAGTCACCTTCTGCGTGAGAACACAGAGCTGGCTCAGC
CTCTGAGGAAAGAACCTGAAATAATCACTGTGACCCTAAAAAAGCAGAAT
GGAATGGGCCTTAGCATTGTTGCAGCAAAGGGTGCTGGTCAAGATAAACT
AGGAATCTACGTGAAGTCGGTTGTGAAAGGAGGTGCTGCAGATGTGGATG
GACGTCTAGCTGCAGGTGATCAGCTCCTCAGTGTGGATGGACGAAGTCTG
GTTGGACTCTCTCAGGAAAGGGCGGCAGAACTCATGACAAGAACAAGCTC
TGTGGTGACACTGGAAGTAGCAAAGCAGGGTGCCATCTACCACGGTCTGG
CCACCCTTCTCAATCAGCCATCCCCCATGATGCAGAGAATTTCAGATCGT
CGTGGCTCAGGTAAACCCCGACCAAAGAGTGAAGGCTTTGAGCTCTATAA
TAATTCAACTCAAAATGGGTCTCCTGAGAGTCCTCAGCTGCCTTGGGCAG
AATATAGTGAACCAAAGAAATTGCCTGGTGATGACAGACTGATGAAAAAT
AGAGCTGATCACCGTTCCAGCCCCAACGTAGCAAATCAGCCTCCTAGTCC
TGGAGGGAAAAGTGCATATGCCTCTGGAACAACAGCGAAGATAACATCTG
TCTCTACTGGAAACCTCTGCACTGAGGAGCAGACGCCTCCGCCTAGACCT
GAAGCCTACCCCATTCCCACTCAGACGTACACCAGAGAGTATTTTACCTT
CCCAGCTTCCAAATCCCAGGATCGGATGGCTCCTCCTCAGAACCAGTGGC
CAAATTATGAGGAAAAGCCACATATGCACACAGATAGTAATCATTCCAGT
ATTGCAATTCAGCGTGTTACACGTTCCCAAGAAGAACTTCGAGAAGATAA
AGCTTACCAACTTGAGCGGCATCGAATAGAGGCAGCTATGGACCGAAAGT
CTGATAGTGATATGTGGATAAATCAGAGCTCCTCACTGGACTCCAGTACC
TCTAGCCAGGAGCATCTGAACCATTCCTCTAAGTCGGTCACCCCTGCTTC
CACACTGACCAAAAGTGGCCCTGGCCGTTGGAAAACACCAGCAGCCATAC
CGGCCACCCCTGTGGCCGTCTCCCAGCCAATCCGAACAGACCTGCCTCCG
CCACCCCCGCCACCTCCAGTCCACTATGCCGGTGATTTCGATGGAATGTC
CATGGATTTGCCTCTCCCACCACCCCCTTCCGCCAACCAGATAGGGCTGC
CGTCTGCGCAGGTGGCTGCTGCTGAACGGAGAAAGAGAGAAGAACATCAG
CGTTGGTATGAGAAGGAGAAGGCCCCCCTGGAGGAGGAGCGGGAGAGGAA
GCGGAGAGAGCAGGAGAGGAAGTTGGGCCAGATGCGCACTCAGTCCTTAA
ACCCTGCTCCGTTTTCTCCCCTGACTGCACAGCAGATGAAGCCCGAAAAG
CCTTCCACACTCCAGCGGCCACAGGAAACAGTCATTCGGGAGCTGCAGCC
TCAGCAGCAGCCCCGCACGATCGAGCGCAGAGACTTGCAGTACATTACAG
TCAGCAAAGAGGAGCTTTCCTCGGGGGACAGTCTGTCCCCCGACCCGTGG
AAGCGGGACGCCAAGGAGAAGCTGGAGAAGCAGCAGCAGATGCACATCGT
GGACATGCTGAGCAAGGAGATCCAGGAGCTCCAGAGCAAACCGGACCGCA
GCGCCGAGGAGAGCGACCGGCTGCGCAAGCTCATGCTGGAGTGGCAGTTC
CAGAAGAGACTCCAGGAGTCGAAGCAGAAGGACGAAGATGACGAGGAGGA
GGAGGACGATGATGTGGACACCATGCTGATCATGCAGCGCCTGGAGGCTG
AACGAAGAGCGAGGGTAAAGGGGGGAGTGCTTTGGCTGTGCCCATCTGTG
GTCCCTATTTTAGCTTCTGCGTGTTTCCCATGGGGATAG
MLLT3

SEQ ID NO:90

gi|4758719|ref|NM_004529.1|Homo sapiens myeloid/
lymphoid or mixed-lineage leukemia (trithorax
homolog, Drosophila); translocated to, 3 (MLLT3),
mRNA
TTTGGGGCTGAGTTTAATAAGCGAGCGAGCGAGCAAGCGAGCGCGGGGGG
AAAAAGGCAGAGAATGTCCGCCATCTACCCTCCGCTCCTGGGCGCGCTCT
CATTCATAGCAGCCTCTTCATGAATTACAGCTGAGGGGGGGCGGAGGAGG
GGGGGGTACCACACAACACCCCAGCAAACCTCCGGGCCCCCAGGCATGGC
TAGCTCGTGTTCCGTGCAGGTGAAGCTGGAGCTGGGGCACCGCGCCCAGG
TGAGGAAAAAACCCACCGTGGAGGGCTTCACCCACGACTGGATGGTGTTC
GTACGCGGTCCGGAGCACAGTAACATACAGCACTTTGTGGAGAAAGTCGT
CTTCCACTTGCACGAAAGCTTTCCTAGGCCAAAAAGAGTGTGCAAAGATC
CACCTTACAAAGTAGAAGAATCTGGGTATGCTGGTTTCATTTTGCCAATT
GAAGTTTATTTTAAAAACAAGGAAGAACCTAGGAAAGTCCGCTTTGATTA
TGACTTATTCCTGCATCTTGAAGGCCATCCACCAGTGAATCACCTCCGCT
GTGAAAAGCTAACTTTCAACAACCCCACAGAGGACTTTAGGAGAAAGTTG
CTGAAGGCAGGAGGGGACCCTAATAGGAGTATTCATACCAGCAGCAGCAG
CAGCAGCAGCAGTAGCAGCAGCAGCAGCAGCAGCAGCAGCAGCAGTAGCA
GCAGCAGCAGCAGCAGCAGCAGCAGCAGTAGCAGCAGCAGTAGCAGCAGC
AGCAGCAGCAGTAGTACCAGTTTTTCAAAGCCTCACAAATTAATGAAGGA
GCACAAGGAAAAACCTTCTAAAGACTCCAGAGAACATAAAAGTGCCTTCA
AAGAACCTTCCAGGGATCACAACAAATCTTCCAAAGAATCCTCTAAGAAA
CCCAAAGAAAATAAACCACTGAAAGAAGAGAAAATAGTTCCTAAGATGGC
CTTCAAGGAACCTAAACCCATGTCAAAAGAGCCAAAACCAGATAGTAACT
TACTCACCATCACCAGTGGACAAGATAAGAAGGCTCCTAGTAAAAGGCCG
CCCATTTCAGATTCTGAAGAACTCTCAGCCAAAAAAAGGAAAAAGAGTAG
CTCAGAGGCTTTATTTAAAAGTTTTTCTAGCGCACCACCACTGATACTCA
CTTGTTCTGCTGACAAAAAACAGATAAAAGATAAATCTCATGTCAAGATG
GGAAAGGTCAAAATTGAAAGTGAGACATCAGAGAAGAAGAAATCAACGTT
ACCGCCATTTGATGATATTGTGGATCCCAATGATTCAGATGTGGAGGAGA
ATATATCCTCTAAATCTGATTCTGAACAACCCAGTCCTGCCAGCTCCAGC
TCCAGCTCCAGCTCCAGCTTCACACCATCCCAGACCAGGCAACAAGGTCC
TTTGAGGTCTATAATGAAAGATCTGCATTCTGATGACAATGAGGAGGAAT
CAGATGAAGTGGAGGATAACGACAATGACTCTGAAATGGAGAGGCCTGTA
AATAGAGGAGGCAGCCGAAGTCGCAGAGTTAGCTTAAGTGATGGCAGCGA
TAGTGAAAGCAGTTCTGCTTCTTCACCCCTACATCACGAACCTCCACCAC
CCTTACTAAAAACCAACAACAACCAGATTCTTGAAGTGAAAAGTCCAATA
AAGCAAAGCAAATCAGATAAGCAAATAAAGAATGGTGAATGTGACAAGGC
ATACCTAGATGAACTGGTAGAGCTTCACAGAAGGTTAATGACATTGAGAG
AAAGACACATTCTGCAGCAGATCGTGAACCTTATAGAAGAAACTGGACAC
TTTCATATCACAAACACAACATTTGATTTTGATCTTTGCTCGCTGGACAA
AACCACAGTCCGTAAACTACAGAGTTACCTGGAAACATCTGGAACATCCT
GAGGATATAACAACTGGATGCATCAAGAACTATTGTGTTTTTTTTTTTTG
GTTTTTTTTTTTTTTGGTTGTGATTTTTTGTTCTTGTTGTTTATATGAAA
ACACTCAAAATGATGCAACCAAAAGGGAAAAAATAAAAATCAAACAACCT
TCAGCTTTATTTTTCTTTAAAGCCAGTCATCATCTCTTGATAAAGGAGAG
GTTAAAGCAAACCAGCCTCAGCGGACCACTCTTCTCTCCAAGGAAATCCC
CGGGAAGAGTTAGCCTGGATAGCCTTGAAAACAAACAAATCAAACACAAC
ACAAGAAAACTCAAAGAATGTGTATGGTATCATGTATCTCTCTGTGGTGG
TTCATTCCACAGGACGAATGCATATTCAACACACTGCCTTATTACATAAC
TGATCTATTTATTATCGCATACAGATATTCTAAGTCGTTGAGGGAATGAC
ACCATCAGACATTATAAGTACTTGGTCCCGTGGATGCTCTTTCAATGCAG
CACCCTTGCCATCCCAAGCCCAGTGACCTTACTCGTATACCGTGCCACTT
TCCACCAACTTTTTCCAAGTCCTTTAACTCGTTGCAGTCTGTATTTTCCA
CCTTTTGTTTTTCCAGTTCCAGGACACAGATTATCAACTGGGGGGACCAA
ATAGCCACCTTGATTTTCTTCTTTGTGGTCTTTTTCCTGAAAGTTGGGGC
CCAGTCCTTGGCTGTATCCATGTAATGATCTTGGACCATGGTAGAAAATG
CACCAAATAGGATCATATGAATTGCTGTCTAGCCTTAGTCAATAAACTTG
TAGGACTTTTAAACAAAAGTGTACCTGTAAATGTCCTGAATCCAGCATTG
TTGAGCTGTCATCAACATTCTTGTGTCTGTTTTACTGTTACAATATTAGG
TGAATATGGAAGTAAAGGCATTCCACAGGATCATCATTTAAAAAAAAAGA
ATTCTGGTCCTGTTTTCTAAAAAAAAAAAACTGTTGTAGAAATTCTTAAT
TTGGATCTATTTATTAGTCAGAGTTTCAGCTTTCTTCAGCTGCCAGTGTG
TTACTCATCTTTATCCTAAAAATCTGGAATCAGAGATTTTTGTTTGTTCA
CATATGATTCTCTTAGACACTTTTATATTTGAAAAAATTAAAATCTTTCT
TTGGGGAAAAATTCTTGGTTATTCTGCCATAACAGATTATGTATTAACTT
GTAGATTCAGTGGTTCAATACCTGTTTAGTTGCTTGCTAATATTTCCAGA
AGGATTTCTTGTATTGGTGAAAGACGGTTGGGGATGGGGGGATTTTTTTG
TTCTTGTTGTACCCTTGTTTTGAAACTAGAAATCTGTCCTGTGGCATGCA
AAAGAAAGCAAATTATTTTTAAAAGAAAAAAACCAAAGTACTTTTGGTGT
CATTATTCCATCTTCTCCATAAGTGGAGAAATGAAAAGTAAGAACAGCTC
ATCTTCAAAGTTTTTACTAGAAATTC
MLLT2

SEQ ID NO:91

>gi|5174572|ref|NM_005935.1|Homo sapiens myeloid/
lymphoid or mixed-lineage leukemia (trithorax
homolog, Drosophila); translocated to, 2 (MLLT2),
mRNA
GGCAATTTCTTTTCCTTTCTAACTGTGGCCCGCGTTGTGCTGTTGCTGGG
CAGGCGTTGGGCGCCGGCGGTCTTCGAGCGTGGGGGCCCGCTGGCTTTCC
CTTCTCAGAAACTGCGCCGGGGGCGCTCGCTTGCCCCGGATTCGGACGCG
GCGCTCCCCGGGCTCGTCTGAAGTGCAGATCGCCGCAGAGGCCCCAGTGC
CCGGATGTCCATCAGGATTAGCGCGAGCCAATACGGGCCGAGCCCGGGGC
TGCGCCGAGGACGCCCGGGGCTCGAGAGCAGGTAGTCCCGTAACATCGGG
GCGCCGCGCCGGGACGCGTCCCCGCCCGGCTCCGCCAAATGGTGAGCGCG
GCGCTGGCAGCAGGGCCCGCGGGGTGAAGGCGCTCATGGACGGAAGACCC
CTGGCTCTATAAGCTGAATTATGGCAGCCCAGTCAAGTTTGTACAATGAC
GACAGAAACCTGCTTCGAATTAGAGAGAAGGAAAGACGCAACCAGGAAGC
CCACCAAGAGAAAGAGGCATTTCCTGAAAAGATTCCCCTTTTTGGAGAGC
CCTACAAGACAGCAAAAGGTGATGAGCTGTCTAGTCGAATACAGAACATG
TTGGGAAACTACGAAGAAGTGAAGGAGTTCCTTAGTACTAAGTCTCACAC
TCATCGCCTGGATGCTTCTGAAAATAGGTTGGGAAAGCCGAAATATCCTT
TAATTCCTGACAAAGGGAGCAGCATTCCATCCAGCTCCTTCCACACTAGT
GTCCACCACCAGTCCATTCACACTCCTGCGTCTGGACCACTTTCTGTTGG
CAACATTAGCCACAATCCAAAGATGGCGCAGCCAAGAACTGAACCAATGC
CAAGTCTCCATGCCAAAAGCTGCGGCCCACCGGACAGCCAGCACCTGACC
CAGGATCGCCTTGGTCAGGAGGGGTTCGGCTCTAGTCATCACAAGAAAGG
TGACCGAAGAGCTGACGGAGACCACTGTGCTTCGGTGACAGATTCGGCTC
CAGAGAGGGAGCTTTCTCCCTTAATCTCTTTGCCTTCCCCAGTTCCCCCT
TTGTCACCTATACATTCCAACCAGCAAACTCTTCCCCGGACGCAAGGAAG
CAGCAAGGTTCATGGCAGCAGCAATAACAGTAAAGGCTATTGCCCAGCCA
AATCTCCCAAGGACCTAGCAGTGAAAGTCCATGATAAAGAGACCCCTCAA
GACAGTTTGGTGGCCCCTGCCCAGCCGCCTTCTCAGACATTTCCACCTCC
CTCCCTCCCCTCAAAAAGTGTTGCAATGCAGCAGAAGCCCACGGCTTATG
TCCGGCCCATGGATGGTCAAGATCAGGCCCCTAGTGAATCCCCTGAACTG
AAACCACTGCCGGAGGACTATCGACAGCAGACCTTTGAAAAAACAGACTT
GAAAGTGCCTGCCAAAGCCAAGCTCACCAAACTGAAGATGCCTTCTCAGT
CAGTTGAGCAGACCTACTCCAATGAAGTCCATTGTGTTGAAGAGATTCTG
AAGGAAATGACCCATTCATGGCCGCCTCCTTTGACAGCAATACATACGCC
TAGTACAGCTGAGCCATCCAAGTTTCCTTTCCCTACAAAGGACTCTCAGC
ATGTCAGTTCTGTAACCCAAAACCAAAAACAATATGATACATCTTCAAAA
ACTCACTCAAATTCTCAGCAAGGAACGTCATCCATGCTCGAAGACGACCT
TCAGCTCAGTGACAGTGAGGACAGTGACAGTGAACAAACCCCAGAGAAGC
CTCCCTCCTCATCTGCACCTCCAAGTGCTCCACAGTCCCTTCCAGAACCA
GTGGCATCAGCACATTCCAGCAGTGCAGAGTCAGAAAGCACCAGTGACTC
AGACAGTTCCTCAGACTCAGAGAGCGAGAGCAGTTCAAGTGACAGCGAAG
AAAATGAGCCCCTAGAAACCCCAGCTCCGGAGCCTGAGCCTCCAACAACA
AACAAATGGCAGCTGGACAACTGGCTGACCAAAGTCAGCCAGCCAGCTGC
GCCACCAGAGGGCCCCAGGAGCACAGAGCCCCCACGGCGGCACCCAGAGA
GTAAGGGCAGCAGCGACAGTGCCACGAGTCAGGAGCATTCTGAATCCAAA
GATCCTCCCCCTAAAAGCTCCAGCAAAGCCCCCCGGGCCCCACCCGAAGC
CCCCCACCCCGGAAAGAGGAGCTGTCAGAAGTCTCCGGCACAGCAGGAGC
CCCCACAAAGGCAAACCGTTGGAACCAAACAACCCAAAAAACCTGTCAAG
GCCTCTGCCCGGGCAGGTTCACGGACCAGCCTGCAGGGGGAAAGGGAGCC
AGGGCTTCTTCCCTATGGCTCCCGAGACCAGACTTCCAAAGACAAGCCCA
AGGTGAAGACGAAAGGACGGCCCCGGGCCGCAGCAAGCAACGAACCCAAG
CCAGCAGTGCCCCCCTCCAGTGAGAAGAAGAAGCACAAGAGCTCCCTCCC
TGCCCCCTCTAAGGCTCTCTCAGGCCCAGAACCCGCGAAGGACAATGTGG
AGGACAGGACCCCTGAGCACTTTGCTCTTGTTCCCCTGACTGAGAGCCAG
GGCCCACCCCACAGTGGCAGCGGCAGCAGGACTAGTGGCTGCCGCCAAGC
CGTGGTGGTCCAGGAGGACAGCCGCAAAGACAGACTCCCATTGCCTTTGA
GAGACACCAAGCTGCTCTCACCGCTCAGGGACACTCCTCCCCCACAAAGC
TTGATGGTGAAGATCACCCTAGACCTGCTCTCTCGGATACCCCAGCCTCC
CGGGAAGGGGAGCCGCCAGAGGAAAGCAGAAGATAAACAGCCGCCCGCAG
GGAAGAAGCACAGCTCTGAGAAGAGGAGCTCAGACAGCTCAAGCAAGTTG
GCCAAAAAGAGAAAGGGTGAAGCAGAAAGAGACTGTGATAACAAGAAAAT
CAGACTGGAGAAGGAAATCAAATCACAGTCATCTTCATCTTCATCCTCCC
ACAAAGAATCTTCTAAAACAAAGCCCTCCAGGCCCTCCTCACAGTCCTCA
AAGAAGGAAATGCTCCCCCCGCCACCCGTGTCCTCGTCCTCCCAGAAGCC
AGCCAAGCCTGCACTTAAGAGGTCAAGGCGGGAAGCAGACACCTGTGGCC
AGGACCCTCCCAAAAGTGCCAGCAGTACCAAGAGCAACCACAAAGACTCT
TCCATTCCCAAGCAGAGAAGAGTAGAGGGGAAGGGCTCCAGAAGCTCCTC
GGAGCACAAGGGTTCTTCCGGAGATACTGCAAATCCTTTTCCAGTGCCTT
CTTTGCCAAATGGTAACTCTAAACCAGGGAAGCCTCAAGTGAAGTTTGAC
AAACAACAAGCAGACCTTCACATGAGGGAGGCAAAAAAGATGAAGCAGAA
AGCAGAGTTAATGACGGACAGGGTTGGAAAGGCTTTTAAGTACCTGGAAG
CCGTCTTGTCCTTCATTGAGTGCGGAATTGCCACAGAGTCTGAAAGCCAG
TCATCCAAGTCAGCTTACTCTGTCTACTCAGAAACTGTAGATCTCATTAA
ATTCATAATGTCATTAAAATCCTTCTCAGATGCCACAGCGCCAACACAAG
AGAAAATATTTGCTGTTTTATGCATGCGTTGCCAGTCCATTTTGAACATG
GCGATGTTTCGTTGTAAAAAAGACATAGCAATAAAGTATTCTCGTACTCT
TAATAAACACTTCGAGAGTTCTTCCAAAGTCGCCCAGGCACCTTCTCCAT
GCATTGCAAGCACAGGCACACCATCCCCTCTTTCCCCAATGCCTTCTCCT
GCCAGCTCCGTAGGGTCCCAGTCAAGTGCTGGCAGTGTGGGGAGCAGTGG
GGTGGCTGCCACTATCAGCACCCCAGTCACCATCCAGAATATGACATCTT
CCTATGTCACCATCACATCCCATGTTCTTACCGCCTTTGACCTTTGGGAA
CAGGCCGAGGCCCTCACGAGGAAGAATAAAGAATTCTTTGCTCGGCTCAG
CACAAATGTGTGCACCTTGGCCCTCAACAGCAGTTTGGTGGACCTGGTGC
ACTATACACGACAGGGTTTTCAGCAGCTACAAGAATTAACCAAAACACCT
TAATGGAGCCCCAGGTTGATTCAATGCCTTGGGAACTATTTTTGCACATT
GGAAGCCTCAAAAACAGTCCAGACGTTTGTTTCATCAGGACACCAAACTC
TAAAAAAGAAGCACCACGAGATGGCCAGGACATTTGTCCACTTAAACTCT
CAACAACAGTGTGATCATTGGTTGGACACTGTGGTTATGCAGAAGCAGAG
ATGAGGAGGCTGGCCCCAGAGATGATCTTGCCCTTCCTAACTAAAGGACA
GAAGTGCAATTTAGCTTAAATGGGTGTATGAATGGTCTAGAAACATTTCT
ATTTTTTTTTTAAACCAGCAGGATACAAGTTGCAAATGAAATGAGGAGAA
ACAGTTTCAACTCTGAAAGTGAATTTCACGTCATCTCAGTAGCCACGCTA
GTCCATTCCCAGAAGGAAATTTTTTTTTTTAACAATGACTTTTGGTAAAG
GGTTTTGTGGATGATTTTTTTTCTTTTGAGTTTTGGGAGAAATATTTGTT
TAATAACTTCTAATGGCCATCTGTAAACCATAAGTAATGAAGGACTCCAC
TGTGCCCCACTTTCTGCCAATGAACAGTGGCTTGATAATACCAAGTATTG
TTGTAATTTATAAAATTGAAGGCAACCCCCGCTCCTGCCGCCCCCAATCT
CCCCATTGCCTAGAGCGCTGCACATTGACCCCAGCTCTGACTTCTCATTA
CTGTGCTGAAAGTCAGCCCACGTCGGAGCGGTGAGGAGGAGCCACAGCAC
ATGGGGTGCCACCTCGAGGTCTGCACAGGAGGACTTGGCGCTGCCATTTC
CTACCCCTGCCATTTCCCACCCCTGCTTCAGCGAAAGGGACTCTCTAACA
GGGCAGTCACTGTTGACTCTATTCTGAATTTCCTCCCTTGGGGAAGAAGG
GAACCAACATTTATACCTGACCAGATGGCTAAAGTGCTTTTAAAGTTTTG
TTTAAGTAGAGCTGGAATTTGAGGTGCTGATCTGTGGTCTACAGTTATGT
GGTAACTCATGTTGTCCAGCCAACTCAGAGTTTCGTCAGTGAACAAGAAA
CATGAAATCTGCTTCTTAGAGAGGCTATATTTTTCTGCTACAAATATTTT
ATATTTATAGCAAAACTAGACTTTCAGAGTCCTTGATTGTCTAGGGGAAG
TTAACTCCCTGAGAGGATGTAGAGATTTGGGGTGGTTGATTAGACTTTTG
AAAAACTCATCACCACATGCCTTCACTCCAGAGTGTTCTCAGCTAGATTT
GATTTGGTTGAGGAGGAACTGTGGCCCTCCGTAAGTTATTGCCATAGTGT
ATGCATTAAACCAAGTCCATTTTGAATGACCTAAAATGAAGTAACACAAT
CAGAAATCCCATGTGCCCATAAGCACAGATTTTTCTTTTTCATTGAAACT
TTAAAGGTTATTATTGGAAACATTACTTTGAGTGCAGTGTTTTTAAAAGC
CAATTCTTTTTTATCCCTTTTAGAAGTAGAATTTGCACACTTACTACAAT
TGAGGAGTGTCATCTCTATAACTTTTTCTCCGCCTTTGTCCCATTCTGCC
CCTGGACATGTTTCCTACCAAGCATGTTTCACATTTTCCTATTAGTGGAG
GAGGGAGAACCATATTTATTTATAATGAAGACATCTAAGATCCCTATGAT
GAATGCAGGAACTCTCTTGGTAGTTTGTAAATACACAAAGGGATGTGTCG
AGGGATGGGAGCGATGCTTATCTCTCACAGTGTGAGTGGTCTGTGTGAGG
CTGTTCCTTCAGTTCTTCTCCAGACTGTTCTTTGGTTGTCACTTAAGTCA
GAGGTCTGGTCCCTCATGTTTAGGTGAAAGCCAGAGAATGACAGCTGTAG
TCATATCTGAGCATAAGACCTTGATGTGTGATTCCTGATGACCGGTTTCA
TTTATTCATGTAATAAAGCAAAGGCCCTGGTCCTTTTTAAACTACTAGTT
TTAAAAACCTGTGTTAAATGAACAGTAATTGCCTGGTAGGTTTGGTGTGT
GTGTAGCATTGTGTGTCCATCTGTTATATGTAAAGGACAAGGCACCAGAA
TCAGGCTTTATTTCGATATTGAAGATGTTATTTAACATCTTTCTTTTTTC
CTTACTCCCTTAGCCATCCCCTCCCCTTTTGTCCTATCATTCCCTAGAAC
AAGCCACCTGTCAATTGTGAAGGGTTGTGTTCTTTATGGCAGGTTCTATG
CAGATTGTGCCAGAGCATGTGCGTGTTCTGTTGGCAAGCCACAGTGCTCC
CTTGACTGAAGACATTTCCAGGTAGATTTCTCAGCCAGCTCTAAAACAGA
TTGCTTTTTCAGTGGCCTTACTCTTTGTGGGTTTTTTTTTTTCTCTGAAC
TTGATATAAAGATTTTATTTGTCCCTTGAAAAAGTAACAAATGTGCATAG
ATCAATTTGTACTACTTTGGTCATTGGATATTTCTGATCCTTATTGCATT
GTACCTAAAGGAGAGTAACTAATGGTAACCTTTTTAATAGAGTATGTGAA
AGGTAGTGGCTGATGAATCCTTAACGTTCATAGGGTCTTTTTGCTGTTAC
GGTTGTATATAGAGGTCTGAAGGATTTTTAAAATGATTTGCACTTTTTCA
CTGCATGCTTACAATTCCCAAAGGCAAAATCTGTACTGAGGTAGATCATT
TGAAAGGGCTAGATTATAAAATTAAGCCTTAGAGTATGGAAAGTTCTTAT
AACAATAATAGTACACACTTCAGAGTAAGACAAATGCAAAGCATCTTAAG
GAGTGAAAATAGAGTCTAAATCTTGCCTTTGGCACTACAAGGTGTGTGTG
TGTGTGTGTGTTGTGTGTCTTTAGTAGGAAATGGAAGAACACTGTTTTAT
TTTTTAAAGTGTTTAATGTTTCTGTCCTTTCTGTGAATTATTGAATTTAA
GAGCCCTGCTAAATAATGAAAAAACACTTTACTAAAATTTATCAAATTAT
ACTGGGTTCGGATTGTGAAAACATTGGCCACCTAGTAGCAGTGGTGAGGA
GTGGGAGGGCCCAGCAAGCATTTATCAGAAATAGAATCACAATAGGAGGA
GAATTTGGCTCTCTGATATTATGATTTGATTACAATACTGAATGGGAAAA
GTATCTAATATTTTGTAACAAAAAGACCTTCATATTATCTGTTTTGACCA
AAATATGTAGCTATTTCCCTTACACAGATTGGACCGCACTTATCTCCCTT
GTCCTGTATCCTTTAATTTCAGGTCTCAGGATGTTTAGAAAGCTAAAACC
CCCTACCCCTTTCTGGCTGAAAACTTGCCTTATTTGGTATCTTACACATT
AATGTTACTAGCATCAGGAGCTTACTGTTTTATTATGATTCATCTTCAGT
AATTTTTAGAAGCAAGAAGAAAGCCATTGTGTCCTCTACAATTAACAAAA
CTTATCTCTGATATACAAAGGGATATAAATATATACACTTAAATAGAGAA
AAAGAGGTTGATTGAATTGTGCCTTTGAGTGAACCCAGTTTTTAAATACC
GCTGTGTTTGTTTCGCCATGGCTTCAGGGATGCTACATGGCTCTTGCACC
TTTTACTCCTCTGCTTTATGAAGTTTGAGTTGTATTTGTGCATCTTAAAG
TAGGTTGAGGCTTGAGGCTGGGCTTTCGGGTTTTTTTGTTTTTTGTTTTG
TTTTGTTTTGTTTTGTTTTCTTGTACTTAAACCTGCTTGCTTCCTACCAC
AGATTCTTTATTTTCCCAAACACTACAAAAAAACTTTTAAAACTTTGCCA
TTTCATCTGTTTACACTCTTTGCCACTGATTAGCAGTATTTAAATCTTGC
AAGAATATTTTGTGCTTTCTTTAGAAACACAAGAGTATAGATTTTTCTCA
CTGAAAAGTGAGAGTTACGCATTGCAGCCATGAAGGGATGCTAGGATCAA
TTATGGCAGTACCTTTTTTCCCCTCCTGTTCTTGAGCCAGTTGTCTCTTT
TGTTTTGGGTCCCACTTAGGATTAACGGATGTAAGGTATTTTCCTGTGCC
TTTATTTTGTGTCATTCTATTGGAAGGAGGTGTAACGGCAGAATAGCATC
GTGTTGGGGGTTTTCCTTCAAACACTGCAAGTGATATTGCCACCATGTGA
ACCTCAAATATGCAATCCAGTTGTGTTGGTTTCTCGGTGACTTGGAGTGT
TCATCTCTTCATGAATTGTGAGCACTGACCATGTTCTTCAGTTCTTAATT
ATGGTGAGTTGACAAATACCAACTACTGCTTTTCTTTAGGTGGCTATAAA
TTTCTTACTGTCAGGAGGAAATGACATTATATTCTGTTCCACTGAACGTC
AGAGATCAGCAGGCACTGTACTGGGTAGAGAAGTGCCTATACTTCTCTAC
CTAAGAGGGCAGGAGGGAAACCCTACAGCTCCTTGTGAGCCTATATATTA
GTATATCGGCCTGGAGAGGACAAGGGAATAAGACCACTCATAGTGAGGCT
GGCCAAGCTGCACTGGTCGGACCAGGCAGTGGCTGACCTAAGGAAGGCAA
CTTGCTTTGCTTAAAAGTAGATTTTTTAAGCAATGCTTAACACAGGCAGC
ATTCACCTTTGTTCAGGCCATCGACATGTATTGTTAAAATTACTGCATAT
CCCCCTCAGATATCAAGTATACACTGTTCATGTTGGGGTTGTGTGTGTGT
ATGTGTGTATGTACGCACGCATGTGTCCCAAATCTTGTTTTAATTTTTTT
TTTCTGAATGTGATCATGTTTTGGATAATACCTGAGCAGGGTTGCCTTTT
TTTTATTTATTACCATTATATATTATATTATATTATATATTTTTTGCTTT
CTTATAACTTTGGAGGAAAGTCAAATCTTGGTATTATTAAAATTGTTTTA
AAAAGGAGTAAATTTTCCAGTTGATAAATGAAAATCACTGGCCTATGTTT
AATAAGTTTTTCTTTAATTACTGTGGAATAACGTGCCAGCTATCATCAAC
ACAATGATTTTGTACATAGGGTAGGGAAGCAGTGATGCTCTCAATGGGAA
GATGTGCAACACAAATTAAGGGGAACTCCATGTATTTTACCTACTTCAGC
AATGGAACTGCAACTTGGGGCTTTGTGAATAAAATTTAGCTGCCTTGTAT
AGTCGTTTGAAAGAATATGTGATCTGTGAGAGAATTATAGTTTTTTTTTA
GAAGAAAAATCTGCAAAAGATCTTTCCAAAGACAATGTGCCACAGATCTT
TTGTTCTCTGTAATGAGGATTAATTGCTGTTTAAACAAAAATGTAATTGT
TCATCTTTAAATTCTTTCCTTTTCATAAGAGGATCAAGCTGTAAAAAAAC
AAAAAAATTAATAAAAATTTCGAGAAATCAAAAAAAAAAA
MLLT10

SEQ ID NO:92

>gi|4757725|ref|NM_004641.1|Homo sapiens myeloid/
lymphoid or mixed-lineage leukemia (trithorax
homolog, Drosophila); translocated to, 10
(MLLT10), mRNA
GCCCTCTTGATTATGTGTGCCCTCTCCGGGCGCCCGCGTTAGCGGCCGGG
TGGAGGTGGGGAGGGAAGACGCTGAGGAGGAGGAGGAGGCGGAGGAGGCG
GTGGAGGGGAGGTGGGGGGAATCAGCAAGGACATGGCTCCTGACTCCTGT
GCGGAACGTGAGTGACTGAGCGGCAAAGCCCGAATGGTCTCTAGCGACCG
GCCCGTGTCACTGGAGGACGAGGTCTCCCATAGTATGAAGGAGATGATTG
GAGGCTGTTGCGTTTGCTCAGACGAGAGAGGCTGGGCCGAGAACCCGCTG
GTTTATTGCGACGGGCACGGCTGCAGCGTCGCGGTGCATCAAGCTTGCTA
TGGCATTGTTCAAGTACCCACTGGACCGTGGTTTTGCAGGAAATGTGAAT
CTCAGGAGAGAGCAGCCAGAGTGAGATGTGAACTTTGTCCCCATAAGGAT
GGAGCTTTAAAAAGAACAGATAATGGGGGTTGGGCCCATGTGGTTTGTGC
CCTGTATATTCCAGAGGTACAATTTGCCAATGTTTCCACAATGGAACCAA
TTGTTTTACAGTCTGTTCCGCATGATCGTTATAATAAGACTTGCTACATT
TGTGATGAACAAGGAAGAGAAAGCAAAGCAGCCACTGGTGCTTGCATGAC
ATGTAATAAACATGGATGTCGACAGGCTTTCCATGTAACATGCGCTCAGT
TTGCCGGACTGCTTTGTGAAGAAGAAGGTAATGGTGCCGATAATGTCCAA
TACTGTGGCTACTGTAAATACCATTTTAGTAAGCTGAAAAAGAGCAAACG
GGGATCTAATAGGTCATATGATCAAAGTTTAAGTGATTCTTCCTCTCACT
CTCAGGATAAACATCATGAGAAAGAGAAAAAAAAATATAAAGAGAAGGAC
AAACACAAACAGAAACACAAGAAGCAGCCAGAACCATCACCTGCATTGGT
TCCATCCTTGACTGTTACTACAGAAAAAACTTATACAAGCACTAGCAACA
ACTCTATATCTGGATCATTGAAGCGCTTGGAAGATACTACTGCACGATTT
ACAAATGCAAATTTCCAGGAAGTCTCTGCACACACCTCTAGTGGAAAAGA
TGTTTCAGAGACTAGAGGGTCAGAGGGCAAAGGGAAGAAATCTTCAGCTC
ACAGCTCAGGTCAAAGGGGAAGAAAGCCTGGTGGTGGAAGAAATCCAGGA
ACAACTGTGTCAGCAGCTAGCCCTTTTCCTCAAGGCAGTTTTTCAGGAAC
TCCAGGCAGTGTAAAGTCATCTTCTGGAAGTTCAGTGCAGTCTCCCCAGG
ATTTCCTGAGCTTTACAGACTCAGATCTGCGTAATGACAGTTACTCTCAC
TCCCAACAGTCATCAGCAACCAAAGATGTACATAAAGGAGAGTCTGGAAG
CCAGGAAGGGGGGGTAAATAGTTTTAGTACCTTAATTGGCCTCCCTTCAA
CCTCAGCTGTTACTTCACAGCCTAAAAGCTTTGAAAATTCACCTGGAGAT
TTGGGTAATTCCAGCCTTCCTACAGCAGGATATAAGCGGGCTCAAACTTC
TGGCATAGAAGAAGAAACTGTAAAGGAAAAGAAAAGGAAAGGAAATAAAC
AAAGTAAGCATGGGCCTGGCAGACCCAAAGGAAACAAAAATCAAGAGAAT
GTTTCTCATCTCTCAGTTTCTTCTGCTTCACCAACATCATCTGTAGCATC
AGCTGCAGGAAGCATAACAAGCTCTAGTCTGCAGAAATCTCCTACATTGC
TCAGGAATGGAAGTTTACAGAGCCTCAGTGTTGGCTCATCTCCAGTTGGT
TCAGAAATTTCCATGCAGTATCGGCATGATGGAGCTTGCCCAACAACTAC
GTTCTCAGAGTTGCTGAATGCAATACACAACGACAGAGGTGACAGTTCTA
CACTAACAAAGCAAGAACTTAAATTCATAGGTATTTATAACAGCAATGAT
GTAGCAGTATCGTTTCCAAATGTAGTATCTGGCTCGGGATCTAGTACTCC
TGTCTCCAGCTCTCACTTACCTCAGCAGTCTTCTGGGCATTTGCAACAAG
TAGGAGCGCTCTCTCCCTCAGCTGTGTCATCTGCAGCCCCTGCTGTTGCT
ACAACTCAGGCAAATACTCTATCTGGATCTTCTCTCAGTCAGGCACCATC
TCATATGTATGGCAATAGATCAAATTCATCAATGGCAGCTCTTATAGCTC
AGTCTGAAAACAATCAAACAGATCAAGATCTTGGAGACAATAGCCGCAAC
CTAGTTGGCAGAGGAAGCTCACCCCGAGGAAGTCTCTCGCCACGATCCCC
TGTAAGCAGCTTACAGATTCGCTATGATCAACCAGGCAACAGCAGTTTGG
AAAATCTGCCTCCAGTAGCAGCCAGCATAGAACAGCTTTTGGAGAGGCAG
TGGAGTGAAGGACAGCAATTTTTACTAGAACAGGGTACTCCTAGTGACAT
TTTAGGAATGCTGAAGTCATTACACCAACTTCAAGTTGAAAACCGAAGAT
TAGAGGAACAAATTAAAAACTTGACTGCCAAAAAGGAACGGCTTCAGTTA
TTGAATGCACAGCTTTCAGTGCCTTTTCCAACAATAACAGCAAATCCTAG
TCCGTCTCATCAAATACACACATTTTCAGCACAGACTGCTCCTACTACTG
ATTCCTTGAACAGCAGTAAGAGCCCTCATATAGGAAACAGCTTTTTACCT
GATAATTCTCTTCCTGTATTAAATCAGGACTTAACCTCCAGTGGACAAAG
TACCAGCAGCTCATCAGCTCTTTCTACCCCACCTCCTGCTGGGCAGAGTC
CGGCTCAACAAGGCTCAGGAGTGAGTGGAGTTCAGCAGGTCAATGGCGTG
ACAGTGGGGGCACTAGCTAGTGGAATGCAGCCTGTAACTTCCACCATTCC
TGCCGTGTCTGCAGTGGGTGGAATAATTGGAGCTTTGCCAGGTAACCAAC
TGGCAATTAATGGCATTGTAGGAGCTTTAAATGGGGTTATGCAGACTCCT
GTCACAATGTCCCAGAACCCTACCCCTCTCACCCACACAACCGTACCACC
TAATGCAACACATCCAATGCCAGCTACACTGACTAACAGTGCCTCAGGAC
TAGGATTACTTTCTGACCAGCAACGACAAATACTTATTCATCAACAGCAG
TTTCAGCAGTTGTTAAATTCTCAACAGCTCACACCAGTACACAGGCACCC
CCACTTCACACAGCTACCACCAACCCATTTCTCACCATCCATGGAGATAA
TGCAAGTCAGAAAGTAGCAAGACTTAGTGATAAAACTGGGCCTGTAGCTC
AAGAGAAAAGTTGACACCTGAGAAACATCTAGAAATTGCCTATCCTGCTG
TTCTAGCACTTCATCTGGCTGCCTTTGCAGTCCTTTTACTACAGCTATGA
AGAAACGCAACAAGAAACTCAATGCACAACAAAGGATTAATTGCTGCAAG
GACATTCTTGTAAGGCTTTGATTAGTTTTCTTGTTGCTTTGTTGCACTGA
AATGGAATTCCCATGCCCCTACCCCTTACCCCAGTTTTTTGAACATGGAA
AGAAAATTTAATAACTTTTTAAAGTGACATAATTTACATGCAATATGTTT
ATCAACTCAAGAATTTAATATAGTTGGTACACAACTAGTTTTGTTTATAA
ATTGGAGATGCAAATAGCAAAACTAAATACTTGCTCCATTTACAAACTAC
TTGATTTTATTGTACAAGTTGAAATATGCTCTTTTGTTTGGGTTACAGTA
TGCTTGCTCTAAGTCAAATTCCAAGGAACTAATTTCTTCTCCTGGAGTTG
CATTGATTCAGTATTACAAATATATAGCACATCACCTGGGAC
MLL2

SEQ ID NO:93

>gi|4505196|ref|NM_003482.1|Homo sapiens myeloid/
lymphoid or mixed-lineage leukemia 2 (MLL2), mRNA
ATGGACAGCCAGAACCTGGCTGGTGAGGATAAAGATTCACAACCGGCAGC
TGATGGACCTGCAGCCTCTGAGGACCCAAGTGCCACTGAGTCAGACCTGC
CCAACCCACATGTGGGAGAGGTCTCTGTCCTTAGTTCTGGGAGTCCCAGG
CTTCAGGAGACTCCTCAGGACTGCAGTGGGGGTCCGGTGCGGCGTTGTGC
TCTCTGTAACTGCGGGGAGCCCGCTCTACACGGGCAGCGGGAGCTACGGC
GCTTTGAGTTGCCATTTGATTGGCCCCGGTGTCCAGTGGTGTCCCCTGGG
GGGAGCCCAGGGCCCAATGAGGCAGTGCTGCCCAGTGAGGACCTATCACA
GATTGGTTTCCCTGAGGGCCTTACACCTGCCCACCTAGGAGAACCTGGAG
GGTCCTGCTGGGCTCACCATTGGTGTGCTGCATGGTCGGCAGGCGTATGG
GGGCAGGAGGGCCCACAACTATGTGGTGTGGACAAGGCCATCTTCTCAGG
GATCTCACAGCGCTGCTCCCACTGCACCAGGCTCGGTGCCTCCATCCCTT
GCCGCTCACCTGGATGTCCACGGCTTTACCACTTCCCCTGCGCGACTGCC
AGCGGTTCCTTCCTATCCATGAAAACACTGCAGCTGCTATGCCCAGAGCA
CAGTGAGGGGGCTGCATATCTGGAGGAGGCTCGCTGTGCAGTGTGTGAGG
GGCCAGGGGAGCTGTGTGACCTGTTCTTCTGTACCAGCTGTGGGCATCAC
TATCACGGGGCCTGCCTGGACACTGCTCTGACTGCCCGCAAACGTGCTGG
CTGGCAGTGCCCTGAATGCAAAGTGTGCCAAGCCTGCAGGAAACCTGGGA
ATGACTCTAAGATGTTGGTTTGCGAGACGTGTGACAAAGGATACCATACT
TTCTGCCTAAAACCACCCATGGAGGAACTGCCTGCTCACTCTTGGAAGTG
CAAGGCGTGCCGGGTGTGCCGGGCCTGTGGGGCGGGCTCAGCAGAACTGA
ATCCCAACTCGGAGTGGTTTGAGAACTACTCTCTCTGTCACCGCTGTCAC
AAAGCCCAGGGAGGTCAGACTATCCGCTCCGTTGCTGAGCAGCATACCCC
GGTGTGTAGCAGATTTTCACCCCCAGAGCCTGGCGATACCCCCACTGACG
AGCCCGATGCTCTGTACGTTGCATGCCAAGGGCAGCCAAAGGGTGGGCAC
GTGACCTCTATGCAACCCAAGGAACCAGGGCCCCTGCAATGTGAAGCCAA
ACCACTAGGGAAAGCAGGGGTCCAACTTGAGCCCCAGTTGGAGGCCCCCC
TAAACGAGGAGATGCCACTGCTGCCCCCACCTGAGGAGTCACCCCTGTCC
CCACCACCTGAGGAATCACCCACGTCCCCACCACCTGAGGCATCACGCCT
GTCGCCACCACCTGAGGAATTGCCCGCATCCCCACTTCCTGAGGCATTGC
ACCTGTCCCGGCCGCTGGAGGAATCGCCCCTCTCTCCGCCGCCTGAGGAG
TCTCCTCTGTCTCCCCCACCTGAATCATCACCTTTTTCTCCACTGGAGGA
GTCGCCCTTGTCTCCACCGGAAGAGTCACCCCCATCTCCTGCACTTGAGA
CGCCTCTATCCCCACCACCTGAAGCATCGCCCCTGTCCCCACCATTTGAA
GAATCTCCTTTGTCCCCGCCACCTGAGGAATTGCCCACTTCCCCGCCACC
TGAAGCATCTCGCCTGTCTCCACCACCTGAGGAGTCACCCATGTCCCCTC
CACCTGAAGAGTCACCCATGTCTCCACCACCGGAGGCATCTCGTCTGTTC
CCACCATTTGAAGAGTCTCCTCTGTCCCCTCCACCTGAGGAGTCTCCCCT
TTCCCCACCACCTGAGGCATCACGCCTGTCCCCACCACCTGAGGACTCGC
CTATGTCCCCACCACCTGAAGAATCACCTATGTCCCCCCCACCTGAGGTA
TCGCGCCTATCCCCCCTGCCTGTGGTGTCACGCCTGTCTCCACCGCCTGA
GGAATCTCCCTTGTCCCCACCGGCCCTGTCTCCTTTGGGGGAGTTAGAGT
ACCCCTTTGGTGCCAAAGGGGACAGTGACCCTGAGTCACCGTTGGCTGCC
CCCATCCTGGAGACACCCATCAGCCCTCCACCAGAAGCTAACTGCACTGA
CCCTGAGCCTGTCCCCCCTATGATCCTTCCCCCATCTCCAGGCTCCCCAG
TGGGGCCGGCTTCTCCCATCCTGATGGAGCCCCTTCCTCCTCAGTGTTCG
CCACTCCTTCAGCATTCCCTGGTTCCCCAAAACTCCCCTCCTTCCCAGTG
CTCTCCTCCTGCCCTACCACTGTCCGTTCCCTCCCCGTTGAGTCCCATAG
GGAAGGTAGTGGGGGTCTCAGATGAGGCTGAGCTGCACGAGATGGAGACT
GAGAAAGTTTCAGAACCTGAATGCCCAGCCTTGGAACCCAGTGCCACCAG
TCCTCTCCCTTCCCCAATGGGGGACCTTTCCTGCCCCGCCCCCAGCCCTG
CCCCAGCCCTGGATGACTTCTCTGGCCTAGGGGAAGACACAGCCCCTCTG
GATGGGATTGATGCTCCGGGTTCACAGCCAGAGCCTGGACAGACCCCTGG
CAGTTTGGCTAGTGAACTTAAAGGCTCCCCTGTGCTCCTGGACCCCGAGG
AGCTGGCCCCTGTGACCCCTATGGAGGTCTACCCCGAATGCAAGCAGACA
GCAGGGCGGGGCTCACCATGTGAAGAACAGGAAGAGCCACGTGCACCGGT
GGCCCCCACACCACCCACTCTCATCAAATCCGACATCGTTAACGAGATCT
CTAATCTGAGCCAGGGTGATGCCAGTGCCAGTTTTCCTGGCTCAGAGCCC
CTCCTGGGCTCTCCAGACCCGGAGGGGGGTGGCTCCCTGTCCATGGAGTT
GGGGGTCTCTACGGATGTTAGTCCAGCCCGAGATGAGGGCTCCCTACGGC
TCTGTACTGACTCACTGCCAGAGACTGATGACTCACTATTGTGCGATGCT
GGGACAGCTATCAGCGGAGGCAAAGCTGAGGGGGAGAAGGGGCGGCGGCG
CAGCTCCCCAGCCCGTTCCCGCATCAAACAGGGTCGCAGCAGCAGTTTCC
CAGGAAGACGCCGGCCTCGTGGAGGAGCCCATGGAGGGCGTGGTAGAGGA
CGGGCCCGGCTAAAGTCAACTGCTTCTTCCATTGAGACTCTGGTAGTTGC
TGACATTGATAGCTCTCCCAGTAAGGAGGAGGAGGAAGAAGATGATGACA
CCATGCAGAATACCGTGGTTCTCTTCTCCAACACAGACAAATTTGTCCTA
ATGCAGGACATGTGTGTGGTATGTGGCAGCTTTGGCCGGGGGGCAGAGGG
CCACCTCCTTGCCTGTTCGCAGTGCTCTCAGTGCTATCACCCTTACTGTG
TCAACAGCAAGATCACCAAGGTGATGCTGCTCAAGGGCTGGCGTTGTGTG
GAGTGTATTGTGTGTGAGGTGTGTGGCCAGGCCTCCGACCCCTCACGCCT
GCTGCTCTGTGATGACTGTGATATTAGCTACCACACATACTGCCTGGACC
CCCCACTGCTCACCGTCCCCAAGGGCGGCTGGAAGTGCAAGTGGTGTGTG
TCCTGTATGCAGTGTGGGGCTGCTTCCCCTGGCTTCCACTGTGAATGGCA
GAATAGTTACACACACTGTGGGCCCTGTGCCAGCCTGGTGACCTGCCCTA
TCTGTCATGCTCCTTACGTAGAAGAGGACCTACTAATCCAGTGCCGCCAC
TGTGAACGGTGGATGCATGCAGGCTGTGAGAGCCTCTTCACAGAGGACGA
TGTGGACCACGCACCCGATGAAGGCTTTGACTGTGTCTCCTGCCAGCCCT
ACGTGGTAAAGCCTGTGGCGCCTGTTGCACCTCCAGAGCTGGTGCCCATG
AAGGTGAAAGAGCCAGAGCCCCAGTACTTTCGCTTCGAAGGCGTGTGGCT
GACAGAAACTGGCATGGCCTTGCTGCGTAACCTGACCATGTCACCACTGC
ACAAGCGGCGCCAACGGCGAGGACGGCTTGGCCTCCCAGGCGAGGCAGGA
TTGGAGGGTTCTGAGCCCTCAGATGCCCTTGGCCCTGATGACAAGAAGGA
TGGGGACCTGGACACCGATGAGCTGCTCAAGGGTGAAGGTGGTGTGGAGC
ACATGGAGTGCGAAATTAAACTGGAGGGCCCCGTCAGCCCTGATGTGGAG
CCTGGCAAAGAGGAGACCGAGGAAAGCAAAAAACGCAAGCGTAAACCATA
TCGGCCTGGCATTGGTGGTTTCATGGTGCGACAGCGGAAATCCCACACAC
GCACGAAAAAGGGGCCTGCTGCACAGGCGGAGGTGTTGAGTGGGGATGGG
CAGCCCGACGAGGTGATACCTGCTGACCTGCCTGCAGAGGGCGCCGTGGA
GCAGAGCTTAGCTGAAGGGGATGAGAAGAAGAAGCAACAGCGGCGAGGGC
GCAAGAGGAGCAAACTGGAGGGCATGTTCCCTGCTTACTTGCAGGAAGCC
TTCTTTGGGAAGGAGCTGCTGGACCTGAGCCGTAAGGCCCTTTTTGCAGT
TGGGGTGGGCCGGCCAAGCTTTGGACTAGGGACCCCAAAAGCCAAGGGAG
ATGGAGGCTCAGAAAGGAAGGAACTCCCCACATCGCAGAAAGGAGATGAT
GGTCCAGATATTGCAGATGAAGAATCCCGTGGCCTCGAGGGCAAAGCCGA
TACACCAGGACCTGAGGATGGGGGCGTGAAGGCATCCCCAGTGCCCAGTG
ACCCTGAGAAGCCAGGCACCCCAGGTGAAGGGATGCTTAGCTCTGACTTA
GACAGGATTTCCACAGAAGAACTGCCCAAGATGGAATCCAAGGACCTGCA
GCAGCTCTTCAAGGATGTTCTGGGCTCTGAACGAGAACAGCATCTGGGTT
GTGGAACCCCTGGCCTAGAAGGCAGCCGTACGCCACTGCAGAGGCCCTTT
CTTCAAGGTGGACTCCCTTTGGGCAATCTGCCCTCCAGCAGCCCAATGGA
CTCCTACCCAGGCCTCTGCCAGTCCCCGTTCCTGGATTCTAGGGAGCGCG
GGGGCTTCTTTAGCCCGGAACCCGGTGAGCCCGACAGCCCCTGGACGGGC
TCAGGTGGCACCACGCCCTCCACCCCCACAACCCCCACCACGGAGGGTGA
GGGCGACGGACTCTCCTATAACCAGCGGAGTCTTCAGCGCTGGGAGAAGG
ATGAGGAGTTGGGCCAGCTGTCCACCATCTCGCCTGTGCTCTATGCCAAC
ATTAATTTTCCTAATCTCAAGCAAGACTACCCAGACTGGTCAAGCCGTTG
CAAACAAATCATGAAGCTCTGGAGAAAGGTTCCAGCAGCTGACAAAGCCC
CCTACCTGCAAAAGGCCAAAGATAACCGGGCAGCTCACCGCATCAACAAG
GTGCAGAAGCAGGCTGAGAGCCAGATCAACAAGCAGACCAAGGTGGGCGA
CATAGCCCGTAAGACTGACCGACCGGCCCTACATCTCCGCATTCCCCCGC
AGCCAGGGGCACTGGGCAGCCCGCCCCCCGCTGCTGCCCCCACCATTTTC
ATTGGCAGCCCCACTACCCCCGCCGGCTTGTCTACCTCTGCGGACGGGTT
CCTGAAGCCGCCGGCGGGCTCGGTGCCTGGCCCTGACTCGCCTGGTGAGC
TCTTCCTCAAGCTCCCACCCCAGGTGCCCGCCCAAGCGCCTTCGCAGGAC
CCCTTTGGACTGGCCCCTGCCTATCCCCTGGAGCCCCGCTTCCCCACGGC
ACCGCCCACCTATCCCCCCTATCCTAGTCCTACGGGGGCCCCTGCGCAGC
CCCCGATGCTGGGCGCCTCATCTCGTCCTGGGGCTGGCCAGCCAGGGGAA
TTCCACACTACCCCACCTGGCACCCCCAGACACCAGCCCTCCACACCTGA
CCCGTTCCTCAAACCCCGCTGCCCCTCGCTGGATAACTTGGCTGTGCCTG
AGAGCCCTGGGGTAGGGGGAGGCAAAGCTTCCGAGCCCCTGCTCTCGCCC
CCACCTTTTGGGGAGTCCCGGAAGGCCCTAGAGGTGAAGAAGGAAGAGCT
TGGGGCATCCTCTCCTAGCTATGGGCCCCCAAACCTGGGCTTTGTTGACT
CACCCTCCTCAGGCACCCACCTGGGTGGCCTGGAGTTAAAGACACCTGAT
GTCTTCAAAGCCCCCCTGACCCCTCGGGCATCTCAGGTAGAGCCCCAGAG
CCCGGGCTTGGGCCTAAGGCCCCAGGAGCCACCCCCTGCCCAGGCTTTGG
CACCTTCTCCTCCAAGTCACCCAGACATCTTTCGCCCTGGCTCCTACACT
GACCCATATGCTCAGCCCCCATTGACTCCTCGGCCCCAACCTCCGCCCCC
TGAGAGCTGCTGTGCTCTGCCCCCTCGCTCACTGCCCTCCGACCCTTTCT
CCCGAGTGCCTGTCAGTCCTCAGTCCCAGTCCAGCTCCCAGTCTCCACTG
ACACCCCGGCCTCTGTCTGCTGAAGCTTTTTGCCCATCACCCGTTACCCC
TCGCTTCCAGTCCCCTGACCCTTATTCTCGCCCACCCTCACGCCCTCAGT
CCCGTGACCCATTTGCCCCATTGCATAAGCCACCCCGACCCCAGCCCCCT
GAAGTTGCCTTTAAGGCTGGGTCTCTAGCCCACACTTCGCTGGGGGCTGG
GGGGTTCCCAGCAGCCCTGCCCGCGGGGCCAGCAGGTGAGCTCCATGCCA
AGGTCCCAAGTGGGCAGCCCCCCAATTTTGTCCGGTCCCCTGGGACGGGT
GCATTTGTGGGCACCCCCTCTCCCATGCGTTTCACTTTCCCTCAGGCAGT
AGGGGAGCCTTCCCTAAAGCCCCCTGTCCCTCAGCCTGGTCTCCCGCCAC
CCCATGGGATCAACAGCCATTTTGGGCCCGGCCCCACCTTGGGCAAGCCT
CAAAGCACAAACTACACAGTAGCCACAGGGAACTTCCACCCATCGGGCAG
CCCCCTGGGGCCCAGCAGCGGGTCCACAGGGGAGAGCTATGGGCTGTCCC
CACTACGCCCTCCGTCGGTTCTGCCACCACCTGCACCCGACGGATCCCTC
CCCTACCTGTCCCATGGAGCCTCACAGCGATCAGGCATCACCTCTCCTGT
CGAAAAGCGAGAAGACCCAGGGACTGGAATGGGTAGCTCTTTGGCGACAG
CTGAACTCCCAGGTACCCAGGACCCAGGCATGTCCGGCCTTAGCCAAACA
GAGCTGGAGAAGCAACGGCAGCGCCAGCGGCTACGAGAGCTGCTGATTCG
GCAGCAGATCCAGCGCAACACCCTGCGGCAGGAGAAGGAAACAGCTGCAG
CAGCTGCGGGAGCAGTGGGGCCTCCAGGCAGCTGGGGTGCTGAGCCCAGC
AGCCCTGCCTTTGAGCAGCTGAGTCGAGGCCAGACCCCCTTTGCTGGGAC
ACAGGACAAGAGCAGCCTTGTGGGGTTGCCCCCAAGCAAGCTGAGTGGCC
CCATCCTGGGGCCAGGGTCCTTCCCTAGCGATGACCGACTCTCCCGGCCA
CCTCCACCAGCCACGCCTTCCTCTATGGATGTGAACAGCCGGCAACTGGT
AGGAGGCTCCCAAGCTTTCTATCAGCGAGCACCCTATCCTGGGTCCCTGC
CCTTACAGCAGCAACAGCAACAACTGTGGCAGCAACAACAGGCAACAGCA
GCAACCTCCATGCGATTTGCCATGTCAGCTCGCTTTCCATCAACTCCTGG
ACCTGAACTTGGCCGCCAAGCCCTAGGTTCCCCGTTGGCGGGAATTTCCA
CCCGTCTGCCAGGCCCTGGTGAGCCAGTGCCTGGTCCAGCTGGTCCTGCC
CAGTTCATTGAGCTGCGGCACAATGTACAGAAAGGACTGGGACCTGGGGG
CACTCCGTTTCCTGGTCAGGGCCCACCTCAGAGACCCCGTTTTTACCCTG
TAAGTGAGGACCCCCACCGACTGGCTCCTGAAGGGCTTCGGGGCCTGGCG
GTATCAGGTCTTCCCCCACAGAAACCCTCAGCCCCACCGGCCCCTGAATT
GAACAACAGTCTTCATCCAACACCCCACACCAAGGGTCCTACCCTGCCAA
CTGGTTTGGAGCTGGTCAACCGGCCCCCGTCGAGCACTGAGCTTGGCCGC
CCCAATCCTCTGGCCCTGGAAGCTGGGAAGTTGCCCTGTGAGGATCCCGA
GCTGGATGACGATTTTGATGCCCACAAGGCCCTAGAGGATGATGAAGAGC
TTGCTCACCTGGGTCTGGGTGTGGATGTGGCCAAGGGTGATGATGAACTT
GGCACCTTAGAAAACCTGGAGACCAATGACCCCCACTTGGATGACCTGCT
CAATGGAGACGAGTTTGACCTGCTGGCATATACTGATCCTGAGCTGGACA
CTGGGGACAAGAAGGATATCTTCAATGAGCACCTGAGGCTGGTAGAATCG
GCTAATGAGGAGGCTGAACGGGAGGCCCTGCTGCGGGGGGTGGAGCCAGG
ACCCTTGGGCCCTGAGGAGCGCCCTCCCCCTGCTGCTGATGCCTCTGAAC
CCCGCCTGGCATCTGTGCTCCCTGAGGTGAAGCCCAAGGTGGAGGAGGGT
GGACGCCACCCTTCTCCTTGCCAATTCACCATTGCTACCCCCAAGGTAGA
GCCCGCACCTGCTGCCAATTCCCTTGGCCTGGGGCTAAAGCCAGGACAGA
GCATGATGGGCAGCCGGGATACCCGGATGGGCACAGGGCCATTTTCTAGC
AGTGGGCACACAGCTGAGAAGGCCTCCTTTGGGGCCACGGGAGGGCCACC
AGCTCACCTGCTGACCCCCAGCCCACTGAGTGGCCCAGGAGGATCCTCCC
TGCTGGAAAAGTTTGAGCTCGAGAGTGGGGCTTTGACCTTGCCTGGTGGA
CCTGCAGCATCTGGGGATGAGCTAGACAAGATGGAGAGCTCACTGGTAGC
CAGCGAGTTACCCCTGCTCATTGAGGACCTGTTGGAGCATGAGAAGAAGG
AGCTGCAGAAGAAGCAGCAGCTTTCAGCACAGTTGCAGCCTGCCCAGCAG
CAGCAGCAACAGCAGCAGCAGCATTCCCTACTGCCTGCACCAGGCCCTGC
CCAGGCCATGTCTTTGCCACATGAGGGCTCTTCTCCCAGTTTGGCTGGGT
CCCAACAGCAGCTTTCCCTGGGTCTTGCAGTTGCCCGACAGCCAGGTTTG
CCCCAGCCACTGATGCCCACCCAGCCACCAGCTCATGCCCTCCAGCAACG
CCTGGCTCCATCCATGGCTATGGTGTCCAATCAAGGGCATATGCTAAGTG
GGCAGCATGGAGGGCAGGCAGGCTTGGTACCCCAGCAGAGCTCACAGCCA
GTGCTATCACAGAAGCCCATGGGCACCATGCCACCTTCCATGTGCATGAA
GCCGCAGCAATTGGCAATGCAGCAGCAGCTGGCAAACAGCTTCTTCCCAG
ATACAGACCTGGACAAATTTGCTGCAGAAGATATCATTGGTCCCATTGCA
AAGGCCAAGATGGTGGCTTTGAAAGGCATCAAGAAAGTGATGGCTCAGGG
CAGCATTGGGGTGGCACCTGGTATGAACAGACAGCAAGTGTCTCTGCTAG
CCCAGAGGCTCTCGGGGGGACCTAGCAGTGATCTGCAGAACCATGTGGCA
GCTGGGAGTGGCCAGGAGCGGAGTGCTGGTGATCCCTCCCAGCCTCGTCC
CAACCCGCCCACTTTTGCTCAGGGAGTGATCAATGAAGCTGACCAGCGGC
AGTATGAGGAGTGGCTGTTCCATACCCAGCAGCTCCTACAGATGCAGCTG
AAGGTGCTAGAGGAGCAGATTGGTGTACACCGCAAGTCCCGGAAGGCTCT
GTGTGCCAAGCAGCGCACTGCCAAAAAAGCTGGCCGTGAGTTCCCAGAAG
CTGATGCTGAGAAGCTCAAGCTGGTTACAGAGCAGCAGAGCAAGATCCAG
AAACAACTGGATCAGGTCCGGAAACAGCAGAAGGAGCACACTAATCTCAT
GGCAGAATATCGGAACAAGCAGCAGCAACAACAGCAGCAGCAGCAGCAAC
AACAGCAACAGCACTCAGCTGTGCTGGCTCTCAGCCCTTCCCAGAGTCCC
CGGCTGCTCACCAAGCTCCCTGGTCAGCTGCTCCCTGGCCATGGGCTGCA
GCCACCACAGGGGCCTCCGGGTGGGCAAGCCGGAGGTCTTCGCCTGACCC
CTGGGGGTATGGCACTACCTGGACAGCCTGGTGGCCCCTTCCTTAATACA
GCTCTGGCCCAACAGCAGCAACAGCAACATTCTGGTGGGGCTGGATCCCT
GGCTGGCCCTTCAGGGGGCTTCTTCCCTGGCAACCTTGCTCTTCGAAGCC
TCGGACCTGATTCAAGGCTTTTACAGGAAAGGCAGCTGCAGCTGCAGCAG
CAACGTATGCAGCTGGCCCAGAAACTGCAGCAGCAGCAGCAGCAGCAACA
GCAGCAGCAGCACCTTCTAGGACAGGTGGCAATCCAGCAGCAACAGCAGC
AGGGTCCTGGAGTACAGACAAACCAAGCTCTGGGTCCCAAGCCCCAGGGC
CTTATGCCTCCCAGCAGCCACCAAGGCCTCCTGGTCCAGCAGCTGTCCCC
TCAACCACCCCAGGGGCCCCAGGGCATGCTGGGCCCTGCCCAGGTGGCTG
TGTTGCAGCAGCAGCACCCTGGAGCTTTGGGCCCCCAGGGCCCTCACAGA
CAGGTGCTTATGACCCAGTCCCGGGTGCTCAGTTCCCCCCAGCTGGCACA
GCAGGGTCAGGGCCTTATGGGACACAGGCTGGTCACAGCCCAGCAGCAGC
AGCAGCAACAACAGCACCAACAGCAAGGGTCCATGGCAGGGCTGTCCCAT
CTTCAGCAAAGTCTGATGTCACACAGTGGGCAGCCCAAACTGAGCGCTCA
GCCCATGGGCTCTTTACAGCAGCTTCAGCAGCAGCAGCAGCTGCAACAGC
AACAGCAACTTCAGCAGCAGCAGCAGCAGCAGCTACAACAGCAACAGCAA
CTTCAGCAGCAACAGCTTCAACAGCAGCAACAGCAGCAGCAGCTTCAACA
ACAGCAGCAGCAACAGCTTCAACAGCAGCAACAGCAGCTACAACAGCAAC
AGCAACAACAACAGCAGCAGTTTCAACAGCAGCAGCAACAGCAGCAGATG
GGCCTTTTAAACCAGAGTCGAACTTTACTGTCCCCTCAGCAACAACAGCA
GCAGCAAGTGGCACTTGGCCCTGGCATGCCAGCAAAGCCTCTTCAACACT
TTTCTAGCCCTGGAGCCCTGGGTCCAACCCTCCTCCTGACGGGCAAGGAA
CAAAACACCGTAGACCCAGCCGTTTCTTCAGAGGCCACTGAGGGGCCCTC
TACACATCAGGGAGGGCCGTTAGCAATAGGAACTACCCCTGAGTCAATGG
CCACTGAACCAGGAGAGGTAAAGCCCTCACTCTCTGGGGACTCACAACTC
CTGCTTGTCCAACCCCAGCCCCAGCCTCAGCCCAGCTCTCTGCAGCTGCA
GCCACCTCTGAGGCTTCCAGGACAACAGCAGCAGCAAGTTAGCCTGCTCC
ACACAGCAGGTGGAGGAAGCCATGGGCAGCTAGGCAGTGGATCATCTTCT
GAGGCCTCATCTGTGCCCCACCTGCTGGCTCAGCCCTCTGTTTCCTTAGG
GGATCAGCCTGGGTCCATGACCCAGAACCTTCTGGGCCCCCAACAGCCCA
TGCTAGAGCGGCCCATGCAAAATAATACAGGGCCACAACCTCCCAAACCA
GGACCTGTCCTCCAGTCTGGGCAGGGTCTGCCTGGGGTTGGAATCATGCC
TACGGTGGGTCAGCTTCGAGCACAGCTCCAAGGAGTCCTGGCCAAAAACC
CACAGCTGCGGCACTTAAGTCCTCAGCAGCAGCAGCAGCTACAGGCACTC
CTCATGCAGCGGCAGCTGCAGCAGAGTCAGGCAGTACGCCAGACCCCACC
CTACCAGGAGCCTGGGACCCAGACCTCTCCCCTCCAGGGCCTCCTGGGCT
GCCAACCTCAACTTGGGGGCTTCCCTGGACCACAGACAGGCCCCCTCCAG
GAGCTAGGGGCAGGGCCTCGACCTCAGGGCCCACCCCGGCTCCCTGCCCC
ACCAGGAGCCTTATCTACAGGACCAGTCCTTGGCCCTGTCCATCCCACAC
CTCCACCATCCAGCCCTCAAGAGCCAAAGAGACCTTCACAATTACCTTCC
CCCAGCTCCCAGCTTCCCACTGAGGCCCAGCTCCCTCCCACCCATCCAGG
GACCCCCAAACCTCAGGGGCCAACCTTGGAGCCGCCTCCTGGGAGGGTCT
GACCTGCTGCTGCCCAGCTTGCAGATACCTTGTTTAGCAAGGGTCTGGGA
CCTTGGGATCCCCCAGACAACCTAGCAGAAACCCAGAAGCCAGAGCAGAG
CAGCCTGGTACCTGGGCATCTGGACCAGGTGAATGGACAGGTGGTGCCTG
AGGCATCCCAACTCAGCATCAAGCAGGAACCTCGGGAAGAGCCATGTGCC
CTGGGAGCCCAGTCAGTGAAGAGGGAGGCCAATGGGGAGCCAATAGGGGC
ACCAGGAACCAGCAACCACCTCCTGCTGGCAGGCCCTCGCTCAGAAGCTG
GGCATCTGCTCTTGCAGAAGCTACTCCGGGCAAAGAATGTGCAACTCAGC
ACTGGGCAGGGGTCCGAGGGGCTGCGAGCTGAGATCAACGGGCACATTGA
CAGCAAGCTGGCTGGGCTGGAGCAGAAACTACAGGGTACCCCCAGCAACA
AGGAGGATGCAGCAGCAAGGAAGCCTTTGACACCGAAGCCCAAGCGGGTA
CAGAAGGCAAGCGACAGGTTGGTGAGCTCCCGAAAGAAGCTGCGGAAGGA
GGACGGCGTCAGGGCCAGCGAGGCCTTGCTGAAACAGCTGAAACAGGAGC
TGTCCCTGCTGCCCCTAACGGAGCCTGCTATCACCGCCAATTTTAGCCTC
TTTGCCCCCTTTGGCAGTGGCTGCCCAGTCAATGGGCAGAGCCAGCTGAG
GGGGGCCTTTGGAAGTGGGGCGCTGCCCACTGGCCCTGACTACTATTCCC
AGCTGCTTACCAAGAATAACCTGAGTAACCCGCCGACACCACCCTCGTCG
CTGCCCCCCACCCCACCCCCATCGGTGCAGCAGAAGATGGTGAATGGCGT
CACCCCATCTGAAGAGCTGGGGGAGCACCCCAAGGATGCTGCCTCTGCCC
GGGATAGTGAAAGGGCACTGAGGGATACTTCAGAGGTGAAGAGTCTAGAC
CTGCTGGCTGCCTTGCCTACACCCCCTCACAATCAGACTGAGGATGTCAG
GATGGAGAGTGATGAGGATAGCGATTCTCCTGACAGCATTGTGCCAGCTT
CATCCCCTGAGAGCATCTTGGGGGAGGAGGCCCCTCGTTTCCCTCATCTG
GGCTCAGGCCGGTGGGAGCAAGAGGACCGGGCCCTCTCCCCTGTCATCCC
CCTCATTCCTCGGGACAGCATCCCAGTCTTCCCAGATACCAAACCTTATG
GGGCCCTTGGCCTGGAGGTCCCTGGAAAGCTGCCTGTCACAACTTGGGAA
AAGGGCAAAGGAAGTGAGGTGTCAGTCATGCTCACAGTCTCTGCTGCTGC
AGACAAGAACCTGAATGGCGTGATGGTGGCAGTGGCGGAGCTGCTGAGCA
TGAAGATCCCCAACTCCTATGAGGTGCTGTTCCCAGAGAGCCCCGCCCGG
GGAGGCACTGAGCCAAAGAAGGGGGAAGCTGAGGGTCCTGGTGGGAAGGA
AAAGGGTCTGGAAGGCAAGAGCCCAGACACTGGCCCTGATTGGCTGAAGC
AGTTTGATGCAGTGTTGGCTGGCTATACCCTGAAGAGGCAACTAGACATC
TTGAGCCTCCTGAAACAGGAGAGCCCCGCCCCAGAGCCACCCACTCAGCA
CAGGTATACCTACAATGTCTCCAATCTGGATGTGCGACAGCTCTCGGCCC
CACCTCCTGAAGAACCCTCCCCGCCCCCTTCCCCCTTGGCACCTTCTCCT
GCCAGTCCCCCTACTGAGCCCTTGGTTGAACTTCCCACCGAACCCTTGGC
TGAGCCACCCGTCCCCTCACCTCTGCCACTGGCCTCATCCCCTGAATCAG
CCCGACCCAAGCCCCGTGCCCGGCCCCCTGAAGAAGGTGAAGATACCCGT
CCTCCTCGCCTCAAGAAATGGAAAGGAGTGCGCTGGAAGCGGCTTCGGCT
GCTGCTGACCATCCAGAAGGGCAGTGGACGGCAGGAGGATGAGCGGGAAG
TGGCAGAGTTTATGGAGCAGCTTGGCACAGCCTTGCGACCTGACAAGGTA
CCGCGAGACATGCGTCGCTGCTGTTTCTGTCATGAGGAGGGTGACGGGGC
CACTGATGGGCCTGCCCGTCTGCTGAACCTGGACCTGGACCTGTGGGTGC
ACCTCAACTGTGCCCTTTGGTCCACGGAGGTGTATGAGACCCAGGGCGGA
GCACTGATGAATGTGGAGGTTGCCCTGCACCGAGGACTGCTAACCAAGTG
CTCCCTGTGCCAGCGAACTGGTGCCACCAGCAGCTGCAATCGCATGCGTT
GCCCCAATGTCTACCATTTTGGTTGTGCCATCCGCGCCAAGTGCATGTTC
TTCAAGGACAAGACCATGCTGTGTCCAATGCATAAGATCAAGGGGCCCTG
TGAGCAAGAGCTGAGCTCTTTTGCTGTCTTCCGGCGGGTCTACATTGAGC
GGGACGAGGTGAAGCAAATCGCTAGCATCATTCAGCGGGGAGAACGGCTG
CACATGTTCCGTGTGGGGGGGCTTGTGTTCCACGCCATCGGACAGCTGCT
GCCTCACCAGATGGCTGACTTTCATAGTGCCACTGCCCTCTATCCCGTGG
GCTACGAGGCCACGCGCATCTATTGGAGCCTCCGCACCAACAATCGTCGC
TGCTGCTATCGCTGTTCTATTGGTGAGAACAACGGGCGGCCGGAGTTTGT
AATCAAAGTCATCGAGCAGGGCCTGGAGGACCTGGTCTTCACTGACGCCT
CTCCCCAGGCCGTGTGGAATCGCATCATTGAGCCTGTGGCTGCCATGAGA
AAAGAGGCTGACATGCTGCGACTCTTCCCTGAGTATCTGAAGGGCGAGGA
GCTCTTTGGGCTGACGGTGCATGCCGTGCTTCGCATAGCTGAATCACTGC
CCGGGGTGGAGAGCTGTCAAAACTATTTATTCCGCTATGGGCGCCACCCC
CTTATGGAGCTGCCACTCATGATCAACCCCACTGGCTGTGCCCGATCAGA
GCCTAAAATCCTCACACACTACAAACGGCCCCATACCCTGAACAGCACCA
GCATGTCTAAGGCATATCAGAGCACCTTCACAGGCGAGACCAACACCCCC
TACAGCAAGCAGTTTGTGCACTCCAAGTCATCTCAGTACCGGCGGCTGCG
CACCGAATGGAAGAACAACGTGTACCTGGCTCGCTCCCGTATCCAGGGCC
TGGGGCTCTATGCAGCCAAGGACCTAGAAAAGCACACAATGGTTATCGAG
TACATTGGCACCATCATTCGGAACGAGGTGGCCAACCGGCGGGAGAAAAT
CTACGAAGAGCAGAATCGAGGCATCTACATGTTCCGAATAAACAATGAAC
ATGTGATTGATGCTACGTTGACCGGCGGCCCTGCCAGGTACATTAACCAT
TCCTGTGCCCCTAACTGTGTGGCCGAAGTCGTGACATTTGACAAAGAGGA
CAAAATCATCATCATCTCCAGCCGGCGAATCCCCAAAGGAGAGGAGCTAA
CCTATGACTATCAGTTTGATTTTGAGGACGATCAGCACGAGATCCCCTGC

TABLE 4
Histone Deacetylases

HDAC1

SEQ ID NO:94

>gi|13128859|ref|NM_004964.2|Homo sapiens histone
deacetylase 1 (HDAC1), mRNA
GAGCGGAGCCGCGGGCGGGAGGGCGGACGGACCGACTGACGGTAGGGACG
GGAGGCGAGCAAGATGGCGCAGACGCAGGGCACCCGGAGGAAAGTCTGTT
ACTACTACGACGGGGATGTTGGAAATTACTATTATGGACAAGGCCACCCA
ATGAAGCCTCACCGAATCCGCATGACTCATAATTTGCTGCTCAACTATGG
TCTCTACCGAAAAATGGAAATCTATCGCCCTCACAAAGCCAATGCTGAGG
AGATGACCAAGTACCACAGCGATGACTACATTAAATTCTTGCGCTCCATC
CGTCCAGATAACATGTCGGAGTACAGCAAGCAGATGCAGAGATTCAACGT
TGGTGAGGACTGTCCAGTATTCGATGGCCTGTTTGAGTTCTGTCAGTTGT
CTACTGGTGGTTCTGTGGCAAGTGCTGTGAAACTTAATAAGCAGCAGACG
GACATCGCTGTGAATTGGGCTGGGGGCCTGCACCATGCAAAGAAGTCCGA
GGCATCTGGCTTCTGTTACGTCAATGATATCGTCTTGGCCATCCTGGAAC
TGCTAAAGTATCACCAGAGGGTGCTGTACATTGACATTGATATTCACCAT
GGTGACGGCGTGGAAGAGGCCTTCTACACCACGGACCGGGTCATGACTGT
GTCCTTTCATAAGTATGGAGAGTACTTCCCAGGAACTGGGGACCTACGGG
ATATCGGGGCTGGCAAAGGCAAGTATTATGCTGTTAACTACCCGCTCCGA
GACGGGATTGATGACGAGTCCTATGAGGCCATTTTCAAGCCGGTCATGTC
CAAAGTAATGGAGATGTTCCAGCCTAGTGCGGTGGTCTTACAGTGTGGCT
CAGACTCCCTATCTGGGGATCGGTTAGGTTGCTTCAATCTAACTATCAAA
GGACACGCCAAGTGTGTGGAATTTGTCAAGAGCTTTAACCTGCCTATGCT
GATGCTGGGAGGCGGTGGTTACACCATTCGTAACGTTGCCCGGTGCTGGA
CATATGAGACAGCTGTGGCCCTGGATACGGAGATCCCTAATGAGCTTCCA
TACAATGACTACTTTGAATACTTTGGACCAGATTTCAAGCTCCACATCAG
TCCTTCCAATATGACTAACCAGAACACGAATGAGTACCTGGAGAAGATCA
AACAGCGACTGTTTGAGAACCTTAGAATGCTGCCGCACGCACCTGGGGTC
CAAATGCAGGCGATTCCTGAGGACGCCATCCCTGAGGAGAGTGGCGATGA
GGACGAAGACGACCCTGACAAGCGCATCTCGATCTGCTCCTCTGACAAAC
GAATTGCCTGTGAGGAAGAGTTCTCCGATTCTGAAGAGGAGGGAGAGGGG
GGCCGCAAGAACTCTTCCAACTTGAAAAAAGCCAAGAGAGTCAAAACAGA
GGATGAAAAAGAGAAAGACCCAGAGGAGAAGAAAGAAGTCACCGAAGAGG
AGAAAACCAAGGAGGAGAAGCCAGAAGCCAAAGGGGTCAAGGAGGAGGTC
AAGTTGGCCTGAATGGACCTCTCCAGCTCTGGCTTCCTGCTGAGTCCCTC
ACGTTTCTTCCCCAACCCCTCAGATTTTATATTTTCTATTTCTCTGTGTA
TTTATATAAAAATTTATTAAATATAAATATCCCCAGGGACAGAAACCAAG
GCCCCGAGCTCAGGGCAGCTGTGCTGGGTGAGCTCTTCCAGGAGCCACCT
TGCCACCCATTCTTCCCGTTCTTAACTTTGAACCATAAAGGGTGCCAGGT
CTGGGTGAAAGGGATACTTTTATGCAACCATAAGACAAACTCCTGAAATG
CCAAGTGCCTGCTTAGTAGCTTTGGAAAGGTGCCCTTATTGAACATTCTA
GAAGGGGTGGCTGGGTCTTCAAGGATCTCCTGTTTTTTTCAGGCTCCTAA
AGTAACATCAGCCATTTTTAGATTGGTTCTGTTTTCGTACCTTCCCACTG
GCCTCAAGTGAGCCAAGAAACACTGCCTGCCCTCTGTCTGTCTTCTCCTA
ATTCTGCAGGTGGAGGTTGCTAGTCTAGTTTCCTTTTTGAGATACTATTT
TCATTTTTGTGAGCCTCTTTGTAATAAAATGGTACATTTCT
HDAC2

SEQ ID NO:95

>gi|4557640|ref|NM_001527.1|Homo sapiens histone
deacetylase 2 (HDAC2), mRNA
CGCCGAGCTTTCGGCACCTCTGCCGGGTGGTACCGAGCCTTCCCGGCGCC
CCCTCCTCTCCTCCCACCGGCCTGCCCTTCCCCGCGGGACTATCGCCCCC
ACGTTTCCCTCAGCCCTTTTCTCTCCCGGCCGAGCCGCGGCGGCAGCAGC
AGCAGCAGCAGCAGCAGGAGGAGGAGCCCGGTGGCGGCGGTGGCCGGGGA
GCCCATGGCGTACAGTCAAGGAGGCGGCAAAAAAAAAGTCTGCTACTACT
ACGACGGTGATATTGGAAATTATTATTATGGACAGGGTCATCCCATGAAG
CCTCATAGAATCCGCATGACCCATAACTTGCTGTTAAATTATGGCTTATA
CAGAAAAATGGAAATATATAGGCCCCATAAAGCCACTGCCGAAGAAATGA
CAAAATATCACAGTGATGAGTATATCAAATTTCTACGGTCAATAAGACCA
GATAACATGTCTGAGTATAGTAAGCAGATGCATATATTTAATGTTGGAGA
AGATTGTCCAGCGTTTGATGGACTCTTTGAGTTTTGTCAGCTCTCAACTG
GCGGTTCAGTTGCTGGAGCTGTGAAGTTAAACCGACAACAGACTGATATG
GCTGTTAATTGGGCTGGAGGATTACATCATGCTAAGAAATACGAAGCATC
AGGATTCTGTTACGTTAATGATATTGTGCTTGCCATCCTTGAATTACTAA
AGTATCATCAGAGAGTCTTATATATTGATATAGATATTCATCATGGTGAT
GGTGTTGAAGAAGCTTTTTATACAACAGATCGTGTAATGACGGTATCATT
CCATAAATATGGGGAATACTTTCCTGGCACAGGAGACTTGAGGGATATTG
GTGCTGGAAAAGGCAAATACTATGCTGTCAATTTTCCAATGTGTGATGGT
ATAGATGATGAGTCATATGGGCAGATATTTAAGCCTATTATCTCAAAGGT
GATGGAGATGTATCAACCTAGTGCTGTGGTATTACAGTGTGGTGCAGACT
CATTATCTGGTGATAGACTGGGTTGTTTCAATCTAACAGTCAAAGGTCAT
GCTAAATGTGTAGAAGTTGTAAAAACTTTTAACTTACCATTACTGATGCT
TGGAGGAGGTGGCTACACAATCCGTAATGTTGCTCGATGTTGGACATATG
AGACTGCAGTTGCCCTTGATTGTGAGATTCCCAATGAGTTGCCATATAAT
GATTACTTTGAGTATTTTGGACCAGACTTCAAACTGCATATTAGTCCTTC
AAACATGACAAACCAGAACACTCCAGAATATATGGAAAAGATAAAACAGC
GTTTGTTTGAAAATTTGCGCATGTTACCTCATGCACCTGGTGTCCAGATG
CAAGCTATTCCAGAAGATGCTGTTCATGAAGACAGTGGAGATGAAGATGG
AGAAGATCCAGACAAGAGAATTTCTATTCGAGCATCAGACAAGCGGATAG
CTTGTGATGAAGAATTCTCAGATTCTGAGGATGAAGGAGAAGGAGGTCGA
AGAAATGTGGCTGATCATAAGAAAGGAGCAAAGAAAGCTAGAATTGAAGA
AGATAAGAAAGAAACAGAGGACAAAAAAACAGACGTTAAGGAAGAAGATA
AATCCAAGGACAACAGTGGTGAAAAAACAGATACCAAAGGAACCAAATCA
GAACAGCTCAGCAACCCCTGAATTTGACAGTCTCACCAATTTCAGAAAAT
CATTAAAAAGAAAATATTGAAAGGAAAATGTTTTCTTTTTGAAGACTTCT
GGCTTCATTTTATACTACTTTGGCATGGACTGTATTTATTTTCAAATGGG
ACTTTTTCGTTTTTGTTTTTCTGGGCAAGTTTTATTGTGAGATTTTCTAA
TTATGAAGCAAAATTTCTTTTCTCCACCATGCTTTATGTGATAGTATTTA
AAATTGATGTGAGTTATTATGTCAAAAAAACTGATCTATTAAAGAAGTAA
TTGGCCTTTCTGAGCTGAAAAAAAAAAAAAAAAAG
HDAC3

SEQ ID NO:96

>gi|13128861|ref|NM_003883.2|Homo sapiens histone
deacetylase 3 (HDAC3), mRNA
GCGGCGGCCGCGGGCGGCGGGCGGCGGAGGTGCGGGGCCTGCTCCCGCCG
GCACCATGGCCAAGACCGTGGCCTATTTCTACGACCCCGACGTGGGCAAC
TTCCACTACGGAGCTGGACACCCTATGAAGCCCCATCGCCTGGCATTGAC
CCATAGCCTGGTCCTGCATTACGGTCTCTATAAGAAGATGATCGTCTTCA
AGCCATACCAGGCCTCCCAGCATGACATGTGCCGCTTCCACTCCGAGGAC
TACATTGACTTCCTGCAGAGAGTCAGCCCCACCAATATGCAAGGCTTCAC
CAAGAGTCTTAATGCCTTCAACGTAGGCGATGACTGCCCAGTGTTTCCCG
GGCTCTTTGAGTTCTGCTCGCGTTACACAGGCGCATCTCTGCAAGGAGCA
ACCCAGCTGAACAACAAGATCTGTGATATTGCCATTAACTGGGCTGGTGG
TCTGCACCATGCCAAGAAGTTTGAGGCCTCTGGCTTCTGCTATGTCAACG
ACATTGTGATTGGCATCCTGGAGCTGCTCAAGTACCACCCTCGGGTGCTC
TACATTGACATTGACATCCACCATGGTGACGGGGTTCAAGAAGCTTTCTA
CCTCACTGACCGGGTCATGACGGTGTCCTTCCACAAATACGGAAATTACT
TCTTCCCTGGCACAGGTGACATGTATGAAGTCGGGGCAGAGAGTGGCCGC
TACTACTGTCTGAACGTGCCCCTGCGGGATGGCATTGATGACCAGAGTTA
CAAGCACCTTTTCCAGCCGGTTATCAACCAGGTAGTGGACTTCTACCAAC
CCACGTGCATTGTGCTCCAGTGTGGAGCTGACTCTCTGGGCTGTGATCGA
TTGGGCTGCTTTAACCTCAGCATCCGAGGGCATGGGGAATGCGTTGAATA
TGTCAAGAGCTTCAATATCCCTCTACTCGTGCTGGGTGGTGGTGGTTATA
CTGTCCGAAATGTTGCCCGCTGCTGGACATATGAGACATCGCTGCTGGTA
GAAGAGGCCATTAGTGAGGAGCTTCCCTATAGTGAATACTTCGAGTACTT
TGCCCCAGACTTCACACTTCATCCAGATGTCAGCACCCGCATCGAGAATC
AGAACTCACGCCAGTATCTGGACCAGATCCGCCAGACAATCTTTGAAAAC
CTGAAGATGCTGAACCATGCACCTAGTGTCCAGATTCATGACGTGCCTGC
AGACCTCCTGACCTATGACAGGACTGATGAGGCTGATGCAGAGGAGAGGG
GTCCTGAGGAGAACTATAGCAGGCCAGAGGCACCCAATGAGTTCTATGAT
GGAGACCATGACAATGACAAGGAAAGCGATGTGGAGATTTAAGAGTGGCT
TGGGATGCTGTGTCCCAAGGAATTTCTTTTCACCTCTTGGTTGGGCTGGA
GGGAAAAGGAGTGGCTCCTAGAGTCCTGGGGGTCACCCCAGGGCTTTTGC
TGACTCTGGGAAAGAGTCTGGAGACCACATTTGGTTCTCGAACCATCTAC
CTGCTTTTCCTCTCTCTCCCAAGGCCTGACAATGGTACCTATTAGGGATG
GAGATACAGACAAGGATAGCTATCTGGGACATTATTGGCAGTGGGCCCTG
GAGGCCAGTCCCTAGCCCCCCTTGCCCCTTATTTCTTCCCTGCTTCCCTC
GAACCCAGAGATTTTTGAGGGATGAACGGGTAGACAAGGACTGAGATTGC
CTCTGACTTCCTCCTCCCCTGGGTTCTGACTTCTTCCTCCCCTTGCTTCC
AGGGAAGATGAAGAGAGAGAGATTTGGAAGGGGCTCTGGCTCCCTAACAC
CTGAATCCCAGATGATGGGAAGTATGTTTTCAAGTGTGGGGAGGATATGA
AAATGTTCTGTTCTCACTTTTGGCTTTATGTCCATTTTACCACTGTTTTT
ATCCAATAAACTAAGTCGGTATTTTTTGTACCTTTAAAAAAAAAAAAAAA
AAAAA
HDAC4

SEQ ID NO:97

>gi|13259519|ref|NM_006037.2|Homo sapiens histone
deacetylase 4 (HDAC4), mRNA
GGAGGTTGTGGGGCCGCCGCCGCGGAGCACCGTCCCCGCCGCCGCCCGAG
CCCGAGCCCGAGCCCGCGCACCCGCCCGCGCCGCCGCCGCCGCCGCCCGA
ACAGCCTCCCAGCCTGGGCCCCCGGCGGCGCCGTGGCCGCGTCCCGGCTG
TCGCCGCCCGAGCCCGAGCCCGCGCGCCGGCGGGTGGCGGCGCAGGCTGA
GGAGATGCGGCGCGGAGCGCCGGAGCAGGGCTAGAGCCGGCCGCCGCCGC
CCGCCGCGGTAAGCGCAGCCCCGGCCCGGCGCCCGCGGGCCATTGTCCGC
CGCCCGCCCCGCGCCCCGCGCAGCCTGCAGGCCTTGGAGCCCGCGGCAGG
TGGACGCCGCCGGTCCACACCCGCCCCGCGCGCGGCCGTGGGAGGCGGGG
GCCAGCGCTGGCCGCGCGCCGTGGGACCCGCCGGTCCCCAGGGCCGCCCG
GCCCCTTCTGGACCTTTCCACCCGCGCCGCGAGGCGGCTTCGCCCGCCGG
GGCGGGGGCGCGGGGGTGGGCACGGCAGGCAGCGGCGCCGTCTCCCGGTG
CGGGGCCCGCGCCCCCCGAGCAGGTTCATCTGCAGAAGCCAGCGGACGCC
TCTGTTCAACTTGTGGGTTACCTGGCTCATGAGACCTTGCCGGCGAGGCT
CGGCGCTTGAACGTCTGTGACCCAGCCCTCACCGTCCCGGTACTTGTATG
TGTTGGTGGGAGTTTGGAGCTCGTTGGAGCTATCGTTTCCGTGGAAATTT
TGAGCCATTTCGAATCACTTAAAGGAGTGGACATTGCTAGCAATGAGCTC
CCAAAGCCATCCAGATGGACTTTCTGGCCGAGACCAGCCAGTGGAGCTGC
TGAATCCTGCCCGCGTGAACCACATGCCCAGCACGGTGGATGTGGCCACG
GCGCTGCCTCTGCAAGTGGCCCCCTCGGCAGTGCCCATGGACCTGCGCCT
GGACCACCAGTTCTCACTGCCTGTGGCAGAGCCGGCCCTGCGGGAGCAGC
AGCTGCAGCAGGAGCTCCTGGCGCTCAAGCAGAAGCAGCAGATCCAGAGG
CAGATCCTCATCGCTGAGTTCCAGAGGCAGCACGAGCAGCTCTCCCGGCA
GCACGAGGCGCAGCTCCACGAGCACATCAAGCAACAACAGGAGATGCTGG
CCATGAAGCACCAGCAGGAGCTGCTGGAACACCAGCGGAAGCTGGAGAGG
CACCGCCAGGAGCAGGAGCTGGAGAAGCAGCACCGGGAGCAGAAGCTGCA
GCAGCTCAAGAACAAGGAGAAGGGCAAAGAGAGTGCCGTGGCCAGCACAG
AAGTGAAGATGAAGTTACAAGAATTTGTCCTCAATAAAAAGAAGGCGCTG
GCCCACCGGAATCTGAACCACTGCATTTCCAGCGACCCTCGCTACTGGTA
CGGGAAAACGCAGCACAGTTCCCTTGACCAGAGTTCTCCACCCCAGAGCG
GAGTGTCGACCTCCTATAACCACCCGGTCCTGGGAATGTACGACGCCAAA
GATGACTTCCCTCTTAGGAAAACAGCTTCTGAACCGAATCTGAAATTACG
GTCCAGGCTAAAGCAGAAAGTGGCCGAAAGACGGAGCAGCCCCCTGTTAC
GCAGGAAAGACGGGCCAGTGGTCACTGCTCTAAAAAAGCGTCCGTTGGAT
GTCACAGACTCCGCGTGCAGCAGCGCCCCAGGCTCCGGACCCAGCTCACC
CAACAACAGCTCCGGGAGCGTCAGCGCGGAGAACGGTATCGCGCCCGCCG
TCCCCAGCATCCCGGCGGAGACGAGTTTGGCGCACAGACTTGTGGCACGA
GAAGGCTCGGCCGCTCCACTTCCCCTCTACACATCGCCATCCTTGCCCAA
CATCACGCTGGGCCTGCCTGCCACCGGCCCCTCTGCGGGCACGGCGGGCC
AGCAGGACACCGAGAGACTCACCCTTCCCGCCCTCCAGCAGAGGCTCTCC
CTTTTCCCCGGCACCCACCTCACTCCCTACCTGAGCACCTCGCCCTTGGA
GCGGGACGGAGGGGCAGCGCACAGCCCTCTTCTGCAGCACATGGTCTTAC
TGGAGCAGCCACCGGCACAAGCACCCCTCGTCACAGGCCTGGGAGCACTG
CCCCTCCACGCACAGTCCTTGGTTGGTGCAGACCGGGTGTCCCCCTCCAT
CCACAAGCTGCGGCAGCACCGCCCACTGGGGCGGACCCAGTCGGCCCCGC
TGCCCCAGAACGCCCAGGCTCTGCAGCACCTGGTCATCCAGCAGCAGCAT
CAGCAGTTTCTGGAGAAACACAAGCAGCAGTTCCAGCAGCAGCAACTGCA
GATGAACAAGATCATCCCCAAGCCAAGCGAGCCAGCCCGGCAGCCGGAGA
GCCACCCGGAGGAGACGGAGGAGGAGCTCCGTGAGCACCAGGCTCTGCTG
GACGAGCCCTACCTGGACCGGCTGCCGGGGCAGAAGGAGGCGCACGCACA
GGCCGGCGTGCAGGTGAAGCAGGAGCCCATTGAGAGCGATGAGGAAGAGG
CAGAGCCCCCACGGGAGGTGGAGCCGGGCCAGCGCCAGCCCAGTGAGCAG
GAGCTGCTCTTCAGACAGCAAGCCCTCCTGCTGGAGCAGCAGCGGATCCA
CCAGCTGAGGAACTACCAGGCGTCCATGGAGGCCGCCGGCATCCCCGTGT
CCTTCGGCGGCCACAGGCCTCTGTCCCGGGCGCAGTCCTCACCCGCGTCT
GCCACCTTCCCCGTGTCTGTGCAGGAGCCCCCCACCAAGCCGAGGTTCAC
GACAGGCCTCGTGTATGACACGCTGATGCTGAAGCACCAGTGCACCTGCG
GGAGTAGCAGCAGCCACCCCGAGCACGCCGGGAGGATCCAGAGCATCTGG
TCCCGCCTGCAGGAGACGGGCCTCCGGGGCAAATGCGAGTGCATCCGCGG
ACGCAAGGCCACCCTGGAGGAGCTACAGACGGTGCACTCGGAAGCCCACA
CCCTCCTGTATGGCACGAACCCCCTCAACCGGCAGAAACTGGACAGTAAG
AAACTTCTAGGCTCGCTCGCCTCCGTGTTCGTCCGGCTCCCTTGCGGTGG
TGTTGGGGTGGACAGTGACACCATATGGAACGAGGTGCACTCGGCGGGGG
CAGCCCGCCTGGCTGTGGGCTGCGTGGTAGAGCTGGTCTTCAAGGTGGCC
ACAGGGGAGCTGAAGAATGGCTTTGCTGTGGTCCGCCCCCCTGGACACCA
TGCGGAGGAGAGCACGCCCATGGGCTTTTGCTACTTCACCTCCGTGGCCG
TGGCAGCCAAGCTTCTGCAGCAGAGGTTGAGCGTGAGCAAGATCCTCATC
GTGGACTGGGACGTGCACCATGGAAACGGGACCCAGCAGGCTTTCTACAG
CGACCCTAGCGTCCTGTACATGTCCCTCCACCGCTACGACGATGGGAACT
TCTTCCCAGGCAGCGGGGCTCCTGATGAGGTGGGCACAGGGCCCGGCGTG
GGTTTCAACGTCAACATGGCTTTCACCGGCGGCCTGGACCCCCCCATGGG
AGACGCTGAGTACTTGGCGGCCTTCAGAACGGTGGTCATGCCGATCGCCA
GCGAGTTTGCCCCGGATGTGGTGCTGGTGTCATCAGGCTTCGATGCCGTG
GAGGGCCACCCCACCCCTCTTGGGGGCTACAACCTCTCCGCCAGATGCTT
CGGGTACCTGACGAAGCAGCTGATGGGCCTGGCTGGCGGCCGGATTGTCC
TGGCCCTCGAGGGAGGCCACGACCTGACCGCCATTTGCGACGCCTCGGAA
GCATGTGTTTCTGCCTTGCTGGGAAACGAGCTTGATCCTCTCCCAGAAAA
GGTTTTACAGCAAAGACCCAATGCAAACGCTGTCCGTTCCATGGAGAAAG
TCATGGAGATCCACAGCAAGTACTGGCGCTGCCTGCAGCGCACAACCTCC
ACAGCGGGGCGTTCTCTGATCGAGGCTCAGACTTGCGAGAACGAAGAAGC
CGAGACGGTCACCGCCATGGCCTCGCTGTCCGTGGGCGTGAAGCCCGCCG
AAAAGAGACCAGATGAGGAGCCCATGGAAGAGGAGCCGCCCCTGTAGCAC
TCCCTCGAAGCTGCTGTTCTCTTGTCTGTCTGTCTCTGTCTTGAAGCTCA
GCCAAGAAACTTTCCCGTGTCACGCCTGCGTCCCACCGTGGGGCTCTCTT
GGAGCACCCAGGGACACCCAGCGTGCAACAGCCACGGGAAGCCTTTCTGC
CGCCCAGGCCCACAGGTCTCGAGACGCACATGCACGCCTGGGCGTGGCAG
CCTCACAGGGAACACGGGACAGACGCCGGCGACGCGCAGACACACGGACA
CGCGGAAGCCAAGCACACTCTGGCGGGTCCCGCAAGGGACGCCGTGGAAG
AAAGGAGCCTGTGGCAACAGGCGGCCGAGCTGCCGAATTCAGTTGACACG
AGGCACAGAAAACAAATATCAAAGATCTAATAATACAAAACAAACTTGAT
TAAAACTGGTGCTTAAAGTTTATTACCCACAACTCCACAGTCTCTGTGTA
AACCACTCGACTCATCTTGTAGCTTATTTTTTTTTTAAAGAGGACGTTTT
CTACGGCTGTGGCCCGCCTCTGTGAACCATAGCGGTGTGCGGCGGGGGGT
CTGCACCCGGGTGGGGGACAGAGGGACCTTTAAAGAAAACAAAACTGGAC
AGAAACAGGAATGTGAGCTGGGGGAGCTGGCTTGAGTTTCTCAAAAGCCA
TCGGAAGATGCGAGTTTGTGCCTTTTTTTTTATTGCTCTGGTGGATTTTT
GTGGCTGGGTTTTCTGAAGTCTGAGGAACAATGCCTTAAGAAAAAACAAA
CAGCAGGAATCGGTGGGACAGTTTCCTGTGGCCAGCCGAGCCTGGCAGTG
CTGGCACCGCGAGCTGGCCTGACGCCTCAAGCACGGGCACCAGCCGTCAT
CTCCGGGGCCAGGGGCTGCAGCCCGGCGGTCCCTGTTTTGCTTTATTGCT
GTTTAAGAAAAATGGAGGTAGTTCCAAAAAAGTGGCAAATCCCGTTGGAG
GTTTTGAAGTCCAACAAATTTTAAACGAATCCAAAGTGTTCTCACACGTC
ACATACGATTGAGCATCTCCATCTGGTCGTGAAGCATGTGGTAGGCACAC
TTGCAGTGTTACGATCGGAATGCTTTTTATTAAAAGCAAGTAGCATGAAG
TATTGCTTAAATTTTAGGTATAAATAAATATATATATGTATAATATATAT
TCCAATGTATTCCAAGCTAAGAAACTTACTTGATTCTTATGAAATCTTGA
TAAAATATTTATAATGCATTTATAGAAAAAGTATATATATATATATAAAA
TGAATGCAGATTGCGAAGGTCCCTGCAAATGGATGGCTTGTGAATTTGCT
CTCAAGGTGCTTATGGAAAGGGATCCTGATTGATTGAAATTCATGTTTTC
TCAAGCTCCAGATTGGCTAGATTTCAGATCGCCAACACATTCGCCACTGG
GCAACTACCCTACAAGTTTGTACTTTCATTTTAATTATTTTCTAACAGAA
CCGCTCCCGTCTCCAAGCCTTCATGCACATATGTACCTAATGAGTTTTTA
TAGCAAAGAATATAAATTTGCTGTTGATTTTTGTATGAATTTTTTCACAA
AAAGATCCTGAATAAGCATTGTTTTATGAATTTTACATTTTTCCTCACCA
TTTAGCAATTTTCTGAATGGTAATAATGTCTAAATCTTTTTCCTTTCTGA
ATTCTTGCTTGTACATTTTTTTTTACCTTTCAAAGGTTTTTAATTATTTT
TGTTTTTATTTTTGTACGATGAGTTTTCTGCAGCGTACAGAATTGTTGCT
GTCAGATTCTATTTTCAGAAAGTGAGAGGAGGGACCGTAGGTCTTTTCGG
AGTGACACCAACGATTGTGTCTTTCCTGGTCTGTCCTAGGAGCTGTATAA
AGAAGCCCAGGGGCTCTTTTTAACTTTCAACACTAGTAGTATTACGAGGG
GTGGTGTGTTTTTCCCCTCCGTGGCAAGGGCAGGGAGGGTTGCTTAGGAT
GCCCGGCCACCCTGGGAGGCTTGCCAGATGCCGGGGGCAGTCAGCATTAA
TGAAACTCATGTTTAAACTTCTCTGACCACATCGTCAGGATAGAATTCTA
ACTTGAGTTTTCCAAAGACCTTTTGAGCATGTCAGCAATGCATGGGGCAC
ACGTGGGGCTCTTTACCCACTTGGGTTTTTCCACTGCAGCCACGTGGCCA
GCCCTGGATTTTGGAGCCTGTGGCTGCAAGGAACCCAGGGACCCTTGTTG
CCTGGTGAACCTGCAGGGAGGGTATGATTGCCTGACCAGGACAGCCAGTC
TTTACTCTTTTTCTCTTCAACAGTAACTGACAGTCACGTTTTACTGGTAA
CTTATTTTCCAGCACATGAAGCCACCAGTTTCATTCCAAAGTGTATATTG
GGTTCAGACTTGGGGGCAGAAGTTCAGACACACCGTGCTCAGGAGGGACC
CAGAGCCGAGTTTCGGAGTTTGGTAAAGTTTACAGGGTAGCTTCTGAAAT
TAACTCAAACTTTTGACCAAATGAGTGCAGATTCTTGGATTCACTTGGTC
ACTGGGCTGCTGATGGTCAGCTCTGAGACAGTGGTTTGAGAGCAGGCAGA
ACGGTCTTGGGACTTGTTTGACTTTCCCCTCCCTGGTGGCCACTCTTTGC
TCTGAAGCCCAGATTGGCAAGAGGAGCTGGTCCATTCCCCATTCATGGCA
CAGAGCAGTGGCAGGGCCCAGCTAGCAGGCTCTTCTGGCCTCCTTGGCCT
CATTCTCTGCATAGCCCTCTGGGGATCCTGCCACCTGCCCTCTTACCCCG
CCGTGGCTTATGGGGAGGAATGCATCATCTCACTTTTTTTTTTTAAGCAG
ATGATGGGATAACATGGACTGCTCAGTGGCCAGGTTATCAGTGGGGGGAC
TTAATTCTAATCTCATTCAAATGGAGACGCCCTCTGCAAAGGCCTGGCAG
GGGGAGGCACGTTTCATCTGTCAGCTCACTCCAGCTTCACAAATGTGCTG
AGAGCATTACTGTGTAGCCTTTTCTTTGAAGACACACTCGGCTCTTCTCC
ACAGCAAGCGTCCAGGGCAGATGGCAGAGGATCTGCCTCGGCGTCTGCAG
GCGGGACCACGTCAGGGAGGGTTCCTTCATGTGTTCTCCCTGTGGGTCCT
TGGACCTTTAGCCTTTTTCTTCCTTTGCAAAGGCCTTGGGGGCACTGGCT
GGGAGTCAGCAAGCGAGCACTTTATATCCCTTTGAGGGAAACCCTGATGA
CGCCACTGGGCCTCTTGGCGTCTGCCCTGCCCTCGCGGCTTCCCGCCGTG
CCGCAGCGTGCCCACGTGCCCACGCCCCACCAGCAGGCGGCTGTCCCGGA
GGCCGTGGCCCGCTGGGACTGGCCGCCCCTCCCCAGCGTCCCAGGGCTCT
GGTTCTGGAGGGCCACTTTGTCAAGGTGTTTCAGTTTTTCTTTACTTCTT
TTGAAAATCTGTTTGCAAGGGGAAGGACCATTTCGTAATGGTCTGACACA
AAAGCAAGTTTGATTTTTGCAGCACTAGCAATGGACTTTGTTGTTTTTCT
TTTTGATCAGAACATTCCTTCTTTACTGGTCACAGCCACGTGCTCATTCC
ATTCTTCTTTTTGTAGACTTTGGGCCCACGTGTTTTATGGGCATTGATAC
ATATATAAATATATAGATATAAATATATATGAATATATTTTTTTAAGTTT
CCTACACCTGGAGGTTGCATGGACTGTACGACCGGCATGACTTTATATTG
TATACAGATTTTGCACGCCAAACTCGGCAGCTTTGGGGAAGAAGAAAAAT
GCCTTTCTGTTCCCCTCTCATGACATTTGCAGATACAAAAGATGGAAATT
TTTCTGTAAAACAAAACCTTGAAGGAGAGGAGGGCGGGGAAGTTTGCGTC
TTATTGAACTTATTCTTAAGAAATTGTACTTTTTATTGTAAGAAAAATAA
AAAGGACTACTTAAACATTTGTCATATTAAGAAAAAAAGTTTATCTAGCA
CTTGTGACATACCAATAATAGAGTTTATTGTATTTATGTGGAAACAGTGT
TTTAGGGAAACTACTCAGAATTCACAGTGAACTGCCTGTCTCTCTCGAGT
TGATTTGGAGGAATTTTGTTTTGTTTTGTTTTGTTTGTTTCCTTTTATCT
CCTTCCACGGGCCAGGCGAGCGCCGCCCGCCCTCACTGGCCTTGTGACGG
TTTATTCTGATTGAGAACTGGGCGGACTCGAAAGAGTCCCCTTTTCCGCA
CAGCTGTGTTGACTTTTTAATTACTTTTAGGTGATGTATGGCTAAGATTT
CACTTTAAGCAGTCGTGAACTGTGCGAGCACTGTGGTTTACAATTATACT
TTGCATCGAAAGGAAACCATTTCTTCATTGTAACGAAGCTGAGCGTGTTC
TTAGCTCGGCCTCACTTTGTCTCTGGCATTGATTAAAAGTCTGCTATTGA
AAGAAAAAG
HDAC5

SEQ ID NO:98

>gi|13259520|ref|NM_005474.2|Homo sapiens histone
deacetylase 5 (HDAC5), mRNA
ATGAACTCTCCCAACGAGTCGGATGGGATGTCAGGTCGGGAACCATCCTT
GGAAATCCTGCCGCGGACTTCTCTGCACAGCATCCCTGTGACAGTGGAGG
TGAAGCCGGTGCTGCCAAGAGCCATGCCCAGTTCCATGGGGGGTGGGGGT
GGAGGCAGCCCCAGCCCTGTGGAGCTACGGGGGGCTCTGGTGGGCTCTGT
GGACCCCACACTGCGGGAGCAGCAACTGCAGCAGGAGCTCCTGGCGCTCA
AGCAGCAGCAGCAGCTGCAGAAGCAGCTCCTGTTCGCTGAGTTCCAGAAA
CAGCATGACCACCTGACAAGGCAGCATGAGGTCCAGCTGCAGAAGCACCT
CAAGCAGCAGCAGGAGATGCTGGCAGCCAAGCAGCAGCAGGAGATGCTGG
CAGCCAAGCGGCAGCAGGAGCTGGAGCAGCAGCGGCAGCGGGAGCAGCAG
CGGCAGGAAGAGCTGGAGAAGCAGCGGCTGGAGCAGCAGCTGCTCATCCT
GCGGAACAAGGAGAAGAGCAAAGAGAGTGCCATTGCCAGCACTGAGGTAA
AGCTGAGGCTCCAGGAATTCCTCTTGTCGAAGTCAAAGGAGCCCACACCA
GGCGGCCTCAACCATTCCCTCCCACAGCACCCCAAATGCTGGGGAGCCCA
CCATGCTTCTTTGGACCAGAGTTCCCCTCCCCAGAGCGGCCCCCCTGGGA
CGCCTCCCTCCTACAAACTGCCTTTGCCTGGGCCCTACGACAGTCGAGAC
GACTTCCCCCTCCGCAAAACAGCCTCTGAACCCAACTTGAAAGTGCGTTC
AAGGCTAAAACAGAAGGTGGCTGAGCGGAGAAGCAGTCCCCTCCTGCGTC
GCAAGGATGGGACTGTTATTAGCACCTTTAAGAAGAGAGCTGTTGAGATC
ACAGGTGCCGGGCCTGGGGCGTCGTCCGTGTGTAACAGCGCACCCGGCTC
CGGCCCCAGCTCTCCCCAACAGTCCCACAGCACCATCGCTGAGAATGGCT
TTACTGGCTCAGTCCCCAACATCCCCACTGAGATGCTCCCTCAGCACCGA
GCCCTCCCTCTGGACAGCTCCCCCAACCAGTTCAGCCTCTACACGTCTCC
TTCTCTGCCCAACATCTCCCTAGGGCTGCAGGCCACGGTCACTGTCACCA
ACTCACACCTCACTGCCTCCCCGAAGCTGTCGACACAGCAGGAGGCCGAG
AGGCAGGCCCTCCAGTCCCTGCGGCAGGGTGGCACGCTGACCGGCAAGTT
CATGAGCACATCCTCTATTCCTGGCTGCCTGCTGGGCGTGGCACTGGAGG
GCGACGGGAGCCCCCACGGGCATGCCTCCCTGCTGCAGCATGTGCTGTTG
CTGGAGCAGGCCCGGCAGCAGAGCACCCTCATTGCTGTGCCACTCCACGG
GCAGTCCCCACTAGTGACGGGTGAACGTGTGGCCACCAGCATGCGGACGG
TAGGCAAGCTCCCGCGGCATCGGCCCCTGAGCCGCACTCAGTCCTCACCG
CTGCCGCAGAGTCCCCAGGCCCTGCAGCAGCTGGTCATGCAACAACAGCA
CCAGCAGTTCCTGGAGAAGCAGAAGCAGCAGCAGCTACAGCTGGGCAAGA
TCCTCACCAAGACAGGGGAGCTGCCCAGGCAGCCCACCACCCACCCTGAG
GAGACAGAGGAGGAGCTGACGGAGCAGCAGGAGGTCTTGCTGGGGGAGGG
AGCCCTGACCATGCCCCGGGAGGGCTCCACAGAGAGTGAGAGCACACAGG
AAGACCTGGAGGAGGAGGACGAGGAAGAGGATGGGGAGGAGGAGGAGGAT
TGCATCCAGGTTAAGGACGAGGAGGGCGAGAGTGGTGCTGAGGAGGGGCC
CGACTTGGAGGAGCCTGGTGCTGGATACAAAAAACTGTTCTCAGATGCCC
AGCCGCTGCAGCCTTTGCAGGTGTACCAGGCGCCCCTCAGCCTGGCCACT
GTGCCCCACCAGGCCCTGGGCCGTACCCAGTCCTCCCCTGCTGCCCCTGG
GGGCATGAAGAGCCCCCCAGACCAGCCCGTCAAGCACCTCTTCACCACAG
GTGTGGTCTACGACACGTTCATGCTAAAGCACCAGTGCATGTGCGGGAAC
ACACACGTGCACCCTGAGCATGCTGGCCGGATCCAGAGCATCTGGTCCCG
GCTGCAGGAGACAGGCCTGCTTAGCAAGTGCGAGCGGATCCGAGGTCGCA
AAGCCACGCTAGATGAGATCCAGACAGTGCACTCTGAATACCACACCCTG
CTCTATGGGACCAGTCCCCTCAACCGGCAGAAGCTAGACAGCAAGAAGTT
GCTCGGCCCCATCAGCCAGAAGATGTATGCTGTGCTGCCTTGTGGGGGCA
TCGGGGTGGACAGTGACACCGTGTGGAATGAGATGCACTCCTCCAGTGCT
GTGCGCATGGCAGTGGGCTGCCTGCTGGAGCTGGCCTTCAAGGTGGCTGC
AGGAGAGCTCAAGAATGGATTTGCCATCATCCGGCCCCCAGGACACCACG
CCGAGGAATCCACAGCCATGGGATTCTGCTTCTTCAACTCTGTAGCCATC
ACCGCAAAACTCCTACAGCAGAAGTTGAACGTGGGCAAGGTCCTCATCGT
GGACTGGGACATTCACCATGGCAATGGCACCCAGCAGGCGTTCTATAATG
ACCCCTCTGTGCTCTACATCTCTCTGCATCGCTATGACAACGGGAACTTC
TTTCCAGGCTCTGGGGCTCCTGAAGAGGTTGGTGGAGGACCAGGCGTGGG
GTACAATGTGAACGTGGCATGGACAGGAGGTGTGGACCCCCCCATTGGAG
ACGTGGAGTACCTTACAGCCTTCAGGACAGTGGTGATGCCCATTGCCCAC
GAGTTCTCACCTGATGTGGTCCTAGTCTCCGCCGGGTTTGATGCTGTTGA
AGGACATCTGTCTCCTCTGGGTGGCTACTCTGTCACCGCCAGATGTTTTG
GCCACTTGACCAGGCAGCTGATGACCCTGGCAGGGGGCCGGGTGGTGCTG
GCCCTGGAGGGAGGCCATGACTTGACCGCCATCTGTGATGCCTCTGAGGC
TTGTGTCTCGGCTCTGCTCAGTGTAGAGCTGCAGCCCTTGGATGAGGCAG
TCTTGCAGCAAAAGCCCAACATCAACGCAGTGGCCACGCTAGAGAAAGTC
ATCGAGATCCAGAGCAAACACTGGAGCTGTGTGCAGAAGTTCGCCGCTGG
TCTGGGCCGGTCCCTGCGAGAGGCCCAAGCAGGTGAGACCGAGGAGGCCG
AGACTGTGAGCGCCATGGCCTTGCTGTCGGTGGGGGCCGAGCAGGCCCAG
GCTGCGGCAGCCCGGGAACACAGCCCCAGGCCGGCAGAGGAGCCCATGGA
GCAGGAGCCTGCCCTGTGACGCCCCGGCCCCCATCCCTCTGGGCTTCACC
ATTGTGATTTTGTTTATTTTTTCTATTAAAAACAAAAAGTCACACATTC
HDAC6

SEQ ID NO:99

>gi|13128863|ref|NM_006044.2|Homo sapiens histone
deacetylase 6 (HDAC6), mRNA
GGGCAGTCCCCTGAGGAGCGGGGCTGGTTGAAACGCTAGGGGCGGGATCT
GGCGGAGTGGAAGAACCGCGGCAGGGGCCAAGCCTCCTCAACTATGACCT
CAACCGGCCAGGATTCCACCACAACCAGGCAGCGAAGAAGTAGGCAGAAC
CCCCAGTCGCCCCCTCAGGACTCCAGTGTCACTTCGAAGCGAAATATTAA
AAAGGGAGCCGTTCCCCGCTCTATCCCCAATCTAGCGGAGGTAAAGAAGA
AAGGCAAAATGAAGAAGCTCGGCCAAGCAATGGAAGAAGACCTAATCGTG
GGACTGCAAGGGATGGATCTGAACCTTGAGGCTGAAGCACTGGCTGGCAC
TGGCTTGGTGTTGGATGAGCAGTTAAATGAATTCCATTGCCTCTGGGATG
ACAGCTTCCCGGAAGGCCCTGAGCGGCTCCATGCCATCAAGGAGCAACTG
ATCCAGGAGGGCCTCCTAGATCGCTGCGTGTCCTTTCAGGCCCGGTTTGC
TGAAAAGGAAGAGCTGATGTTGGTTCACAGCCTAGAATATATTGATCTGA
TGGAAACAACCCAGTACATGAATGAGGGAGAACTCCGTGTCCTAGCAGAC
ACCTACGACTCAGTTTATCTGCATCCGAACTCATACTCCTGTGCCTGCCT
GGCCTCAGGCTCTGTCCTCAGGCTGGTGGATGCGGTCCTGGGGGCTGAGA
TCCGGAATGGCATGGCCATCATTAGGCCTCCTGGACATCACGCCCAGCAC
AGTCTTATGGATGGCTATTGCATGTTCAACCACGTGGCTGTGGCAGCCCG
CTATGCTCAACAGAAACACCGCATCCGGAGGGTCCTTATCGTAGATTGGG
ATGTGCACCACGGTCAAGGAACACAGTTCACCTTCGACCAGGACCCCAGT
GTCCTCTATTTCTCCATCCACCGCTACGAGCAGGGTAGGTTCTGGCCCCA
CCTGAAGGCCTCTAACTGGTCCACCACAGGTTTCGGCCAAGGCCAAGGAT
ATACCATCAATGTGCCTTGGAACCAGGTGGGGATGCGGGATGCTGACTAC
ATTGCTGCTTTCCTGCACGTCCTGCTGCCAGTCGCCCTCGAGTTCCAGCC
TCAGCTGGTCCTGGTGGCTGCTGGATTTGATGCCCTGCAAGGGGACCCCA
AGGGTGAGATGGCCGCCACTCCGGCAGGGTTCGCCCAGCTAACCCACCTG
CTCATGGGTCTGGCAGGAGGCAAGCTGATCCTGTCTCTGGAGGGTGGCTA
CAACCTCCGCGCCCTGGCTGAAGGCGTCAGTGCTTCGCTCCACACCCTTC
TGGGAGACCCTTGCCCCATGCTGGAGTCACCTGGTGCCCCCTGCCGGAGT
GCCCAGGCTTCAGTTTCCTGTGCTCTGGAAGCCCTTGAGCCCTTCTGGGA
GGTTCTTGTGAGATCAACTGAGACCGTGGAGAGGGACAACATGGAGGAGG
ACAATGTAGAGGAGAGCGAGGAGGAAGGACCCTGGGAGCCCCCTGTGCTC
CCAATCCTGACATGGCCAGTGCTACAGTCTCGCACAGGGCTGGTCTATGA
CCAAAATATGATGAATCACTGCAACTTGTGGGACAGCCACCACCCTGAGG
TACCCCAGCGCATCTTGCGGATCATGTGCCGTCTGGAGGAGCTGGGCCTT
GCCGGGCGCTGCCTCACCCTGACACCGCGCCCTGCCACAGAGGCTGAGCT
GCTCACCTGTCACAGTGCTGAGTACGTGGGTCATCTCCGGGCCACAGAGA
AAATGAAAACCCGGGAGCTGCACCGTGAGAGTTCCAACTTTGACTCCATC
TATATCTGCCCCAGTACCTTCGCCTGTGCACAGCTTGCCACTGGCGCTGC
CTGCCGCCTGGTGGAGGCTGTGCTCTCAGGAGAGGTTCTGAATGGTGCTG
CTGTGGTGCGTCCCCCAGGACACCACGCAGAGCAGGATGCAGCTTGCGGT
TTTTGCTTTTTCAACTCTGTGGCTGTGGCTGCTCGCCATGCCCAGACTAT
CAGTGGGCATGCCCTACGGATCCTGATTGTGGATTGGGATGTCCACCACG
GTAATGGAACTCAGCACATGTTTGAGGATGACCCCAGTGTGCTATATGTG
TCCCTGCACCGCTATGATCATGGCACCTTCTTCCCCATGGGGGATGAGGG
TGCCAGCAGCCAGATCGGCCGGGCTGCGGGCACAGGCTTCACCGTCAACG
TGGCATGGAACGGGCCCCGCATGGGTGATGCTGACTACCTAGCTGCCTGG
CATCGCCTGGTGCTTCCCATTGCCTACGAGTTTAACCCAGAACTGGTGCT
GGTCTCAGCTGGCTTTGATGCTGCACGGGGGGATCCGCTGGGGGGCTGCC
AGGTGTCACCTGAGGGTTATGCCCACCTCACCCACCTGCTGATGGGCCTT
GCCAGTGGCCGCATTATCCTTATCCTAGAGGGTGGCTATAACCTGACATC
CATCTCAGAGTCCATGGCTGCCTGCACTCGCTCCCTCCTTGGAGACCCAC
CACCCCTGCTGACCCTGCCACGGCCCCCACTATCAGGGGCCCTGGCCTCA
ATCACTGAGACCATCCAAGTCCATCGCAGATACTGGCGCAGCTTACGGGT
CATGAAGGTAGAAGACAGAGAAGGACCCTCCAGTTCTAAGTTGGTCACCA
AGAAGGCACCCCAACCAGCCAAACCTAGGTTAGCTGAGCGGATGACCACA
CGAGAAAAGAAGGTTCTGGAAGCAGGCATGGGGAAAGTCACCTCGGCATC
ATTTGGGGAAGAGTCCACTCCAGGCCAGACTAACTCAGAGACAGCTGTGG
TGGCCCTCACTCAGGACCAGCCCTCAGAGGCAGCCACAGGGGGAGCCACT
CTGGCCCAGACCATTTCTGAGGCAGCCATTGGGGGAGCCATGCTGGGCCA
GACCACCTCAGAGGAGGCTGTCGGGGGAGCCACTCCGGACCAGACCACCT
CAGAGGAGACTGTGGGAGGAGCCATTCTGGACCAGACCACCTCAGAGGAT
GCTGTTGGGGGAGCCACGCTGGGCCAGACTACCTCAGAGGAGGCTGTAGG
AGGAGCTACACTGGCCCAGACCACCTCGGAGGCAGCCATGGAGGGAGCCA
CACTGGACCAGACTACGTCAGAGGAGGCTCCAGGGGGCACCGAGCTGATC
CAAACTCCTCTAGCCTCGAGCACAGACCACCAGACCCCCCCAACCTCACC
TGTGCAGGGAACTACACCCCAGATATCTCCCAGTACACTGATTGGGAGTC
TCAGGACCTTGGAGCTAGGCAGCGAATCTCAGGGGGCCTCAGAATCTCAG
GCCCCAGGAGAGGAGAACCTACTAGGAGAGGCAGCTGGAGGTCAGGACAT
GGCTGATTCGATGCTGATGCAGGGATCTAGGGGCCTCACTGATCAGGCCA
TATTTTATGCTGTGACACCACTGCCCTGGTGTCCCCATTTGGTGGCAGTA
TGCCCCATACCTGCAGCAGGCCTAGACGTGACCCAACCTTGTGGGGACTG
TGGAACAATCCAAGAGAATTGGGTGTGTCTCTCTTGCTATCAGGTCTACT
GTGGTCGTTACATCAATGGCCACATGCTCCAACACCATGGAAATTCTGGA
CACCCGCTGGTCCTCAGCTACATCGACCTGTCAGCCTGGTGTTACTACTG
TCAGGCCTATGTCCACCACCAGGCTCTCCTAGATGTGAAGAACATCGCCC
ACCAGAACAAGTTTGGGGAGGATATGCCCCACCCACACTAAGCCCCAGAA
TACGGTCCCTCTTCACCTTCTGAGGCCCACGATAGACCAGCTGTAGCTCA
TTCCAGCCTGTACCTTGGATGAGGGGTAGCCTCCCACTGCATCCCATCCT
GAATATCCTTTGCAACTCCCCAAGAGTGCTTATTTAAGTGTTAATACTTT
TAAGAGAACTGCGACGATTAATTGTGGATCTCCCCCTGCCCATTGCCTGC
TTGAGGGGCACCACTACTCCAGCCCAGAAGGAAAGGGGGGCAGCTCAGTG
GCCCCAAGAGGGAGCTGATATCATGAGGATAACATTGGCGGGAGGGGAGT
TAACTGGCAGGCATGGCAAGGTTGCATATGTAATAAAGTACAAGCTGTT
HDAC7

SEQ ID NO:100

>gi|13259521|ref|NM_015401.1|Homo sapiens histone
deacetylase 7A (HDAC7A), transcript variant 1,
mRNA
ATAATACCTACCTTGCAGGACCACGACAGGATTAAGTGAGGAAAAACCCC
CATGAGAGTGTTTTGCCATTGTCAAGTGAGCCTGAGGGAGGCTGAGGGGG
GATCAGGCTGTATCATGCCCCCGAGGACAAACTTTCCAGTTTACCCTGCT
CCCTCTCTCTGTCCCTAGGCTGCCCCAGGCCCTGTGCAGACACACCAGGC
CCTCAGCCGCAGCCCATGGACCTGCGGGTGGGCCAGCGGCCCCCAGTGGA
GCCCCCACCAGAGCCCACATTGCTGGCCCTGCAGCGTCCCCAGCGCCTGC
ACCACCACCTCTTCCTAGCAGGCCTGCAGCAGCAGCGCTCGGTGGAGCCC
ATGAGGCTCTCCATGGACACGCCGATGCCCGAGTTGCAGGTGGGACCCCA
GGAACAAGAGCTGCGGCAGCTTCTCCACAAGGACAAGAGCAAGCGAAGTG
CTGTAGCCAGCAGCGTGGTCAAGCAGAAGCTAGCGGAGGTGATTCTGAAA
AAACAGCAGGCGGCCCTAGAAAGAACAGTCCATCCCAACAGCCCCGGCAT
TCCCTACAGAACCCTGGAGCCCCTGGAGACGGAAGGAGCCACCCGCTCCA
TGCTCAGCAGCTTTTTGCCTCCTGTTCCCAGCCTGCCCAGTGACCCCCCA
GAGCACTTCCCTCTGCGCAAGACAGTCTCTGAGCCCAACCTGAAGCTGCG
CTATAAGCCCAAGAAGTCCCTGGAGCGGAGGAAGAATCCACTGCTCCGAA
AGGAGAGTGCGCCCCCCAGCCTCCGGCGGCGGCCCGCAGAGACCCTCGGA
GACTCCTCCCCAAGTAGTAGCAGCACGCCCGCATCAGGGTGCAGCTCCCC
CAATGACAGCGAGCACGGCCCCAATCCCATCCTGGGCTCGGAGGCGCTCT
TGGGCCAGCGGCTGCGGCTGCAGGAGACTTCTGTGGCCCCGTTCGCCTTG
CCGACAGTGTCCTTGCTGCCCGCAATCACTCTGGGGCTGCCCGCCCCTGC
CAGGGCTGACAGTGACCGCAGGACCCATCCGACTCTGGGCCCTCGGGGGC
CAATCCTGGGGAGCCCCCACACTCCCCTCTTCCTGCCCCATGGCTTGGAG
CCCGAGGCTGGGGGCACCTTGCCCTCTCGCCTGCAGCCCATTCTCCTCCT
GGACCCCTCAGGCTCTCATGCCCCGCTGCTGACTGTGCCCGGGCTTGGGC
CCTTGCCCTTCCACTTTGCCCAGTCCTTAATGACCACCGAGCGGCTCTCT
GGGTCAGGCCTCCACTGGCCACTGAGCCGGACTCGCTCAGAGCCCCTGCC
CCCCAGTGCCACCGCTCCCCCACCGCCGGGCCCCATGCAGCCCCGCCTGG
AGCAGCTCAAAACTCACGTCCAGGTGATCAAGAGGTCAGCCAAGCCGAGT
GAGAAGCCCCGGCTGCGGCAGATACCCTCGGCTGAAGACCTGGAGACAGA
TGGCGGGGGACCGGGCCAGGTGGTGGACGATGGCCTGGAGCACAGGGAGC
TGGGCCATGGGCAGCCTGAGGCCAGAGGCCCCGCTCCTCTCCAGCAGCAC
CCTCAGGTGTTGCTCTGGGAACAGCAGCGACTGGCTGGGCGGCTCCCCCG
GGGCAGCACCGGGGACACTGTGCTGCTTCCTCTGGCCCAGGGTGGGCACC
GGCCTCTGTCCCGGGCTCAGTCTTCCCCAGCCGCACCTGCCTCACTGTCA
GCCCCAGAGCCTGCCAGCCAGGCCCGAGTCCTCTCCAGCTCAGAGACCCC
TGCCAGGACCCTGCCCTTCACCACAGGGCTGATCTATGACTCGGTCATGC
TGAAGCACCAGTGCTCCTGCGGTGACAACAGCAGGCACCCGGAGCACGCC
GGCCGCATCCAGAGCATCTGGTCCCGGCTGCAGGAGCGGGGGCTCCGGAG
CCAGTGTGAGTGTCTCCGAGGCCGGAAGGCCTCCCTGGAAGAGCTGCAGT
CGGTCCACTCTGAGCGGCACGTGCTCCTCTACGGCACCAACCCGCTCAGC
CGCCTCAAACTGGACAACGGGAAGCTGGCAGGGCTCCTGGCACAGCGGAT
GTTTGTGATGCTGCCCTGTGGTGGGGTTGGGGTGGACACTGACACCATCT
GGAATGAGCTTCATTCCTCCAATGCAGCCCGCTGGGCCGCTGGCAGTGTC
ACTGACCTCGCCTTCAAAGTGGCTTCTCGTGAGCTAAAGAATGGTTTCGC
TGTGGTGCGGCCCCCAGGACACCATGCAGATCATTCAACAGCCATGGGCT
TCTGCTTCTTCAACTCAGTGGCCATCGCCTGCCGGCAGCTGCAACAGCAG
AGCAAGGCCAGCAAGATCCTCATTGTAGACTGGGACGTGCACCATGGCAA
CGGCACCCAGCAAACCTTCTACCAAGACCCCAGTGTGCTCTACATCTCCC
TGCATCGCCATGACGACGGCAACTTCTTCCCGGGGAGTGGGGCTGTGGAT
GAGGTAGGGGCTGGCAGCGGTGAGGGCTTCAATGTCAATGTGGCCTGGGC
TGGAGGTCTGGACCCCCCCATGGGGGATCCTGAGTACCTGGCTGCTTTCA
GGATAGTCGTGATGCCCATCGCCCGAGAGTTCTCTCCAGACCTAGTCCTG
GTGTCTGCTGGATTTGATGCTGCTGAGGGTCACCCGGCCCCACTGGGTGG
CTACCATGTTTCTGCCAAATGTTTTGGATACATGACGCAGCAACTGATGA
ACCTGGCAGGAGGCGCAGTGGTGCTGGCCTTGGAGGGTGGCCATGACCTC
ACAGCCATCTGTGACGCCTCTGAGGCCTGTGTGGCTGCTCTTCTGGGTAA
CAGGGTGGATCCCCTTTCAGAAGAAGGCTGGAAACAGAAACCCAACCTCA
ATGCCATCCGCTCTCTGGAGGCCGTGATCCGGGTGCACAGTAAATACTGG
GGCTGCATGCAGCGCCTGGCCTCCTGTCCAGACTCCTGGGTGCCTAGAGT
GCCAGGGGCTGACAAAGAAGAAGTGGAGGCAGTGACCGCACTGGCGTCCC
TCTCTGTGGGCATCCTGGCTGAAGATAGGCCCTCGGAGCAGCTGGTGGAG
GAGGAAGAACCTATGAATCTCTAAGGCTCTGGAACCATCTGCCCGCCCAC
CATGCCCTTGGGACCTGGTTCTCTTCTAACCCCTGGCAATAGCCCCCATT
CCTGGGTCTTTAGAGATCCTGTGGGCAAGTAGTTGGAACCAGAGAACAGC
CTGCCTGCTTTGACAGTTATCCCAGGGAGCGTGAGAAAATCCCTGGGTCT
AGAATGGGAACTGGAGAGGACCCTGAGAGGAGACGGGCTGGGCGGCGACC
CCCACAGGGCTCTCGAGAACAGATTCTCCCCTCCAGTATGGGCCCTGGCT
GTGGCCCCCATTCCTCAGGACTGCACAGAGGAGGACTGGCTCCGGCTCCG
TCGGGCTCACCCTTAACCACTATTCCTGGCTCTGCAAACCCCAGACTTTG
CACACAGCCCCAGGCTCCACACAGAAATGTGAACTTGGCCTCAGACAGGC
TGGCCCTTCCTAGGCTCTAGGGGCTAGGGGGGAGTGGGGAGCCAAGAGGT
CCCATATTCCTGAGTGCAGGGGTAGTCCCTCTCACCTGCTTCCTCAGACG
ACTCTGGAAGCTTCCCTCTACCACCGGGCACTGAGACGAAGCTCCCTGAC
AGCCGAGACTGGCAGCCCTCCATCTGGTCCGTACCCTCGCCAGAGGCCCC
CCTACATCAACCTCCTGGCGATGCCCTGGTGGAGCAGATGGGTGCTCTGG
GAGTCCTGTGCTTCCTGATCCAATGGTGCCAAACCCTTCATCTCCCCCAG
AAGCGCAGCATACCCCTGGGACCCCTCGGCCACTGCCCACTCGGGGAGCC
TTCTCTGTTTCTGGGGCCTCCCCCACCATAGCTCTGATTCCCACCCCACA
TAGGAATAGCCTGACTGAGGGGGAAGGGGTGGGAGAGAAGATACAGACAT
GGAGGAGGGGAGGCTGCTCTGGCAAAGTCTTCAAGGCTTTTGGGGGTCCA
GGCCTGGGGTCAAGAAGGAAAATGTGTGTGAGCATGTGTGTGAGTGAGGC
GTGTGTGTGAGCGTGTGTGTGAGTGAGGCGTGTGTGTGTGTCTTTCCTAG
GACCCACCATACCCTGTGTATGTATGCATGTTTTTGTAAAAAGGAAGAAA
ATGGAAAAAAATCTGAACAATAAATGTTTTATTTGCTTTAAAAAAAAAAA
AAAAAA

SEQ ID NO:101

>gi|13259523|ref|NM_016596.2|Homo sapiens histone
deacetylase 7A (HDAC7A), transcript variant 2,
mRNA
ATAATACCTACCTTGCAGGACCACGACAGGATTAAGTGAGGAAAAACCCC
CATGAGAGTGTTTTGCCATTGTCAAGTGAGCCTGAGGGAGGCTGAGGGGG
GATCAGGCTGTATCATGCCCCCGAGGACAAACTTTCCAGTTTACCCTGCT
CCCTCTCTCTGTCCCTAGGCTGCCCCAGGCCCTGTGCAGACACACCAGGC
CCTCAGCCGCAGCCCATGGACCTGCGGGTGGGCCAGCGGCCCCCAGTGGA
GCCCCCACCAGAGCCCACATTGCTGGCCCTGCAGCGTCCCCAGCGCCTGC
ACCACCACCTCTTCCTAGCAGGCCTGCAGCAGCAGCGCTCGGTGGAGCCC
ATGAGGCTCTCCATGGACACGCCGATGCCCGAGTTGCAGGTGGGACCCCA
GGAACAAGAGCTGCGGCAGCTTCTCCACAAGGACAAGAGCAAGCGAAGTG
CTGTAGCCAGCAGCGTGGTCAAGCAGAAGCTAGCGGAGGTGATTCTGAAA
AAACAGCAGGCGGCCCTAGAAAGAACAGTCCATCCCAACAGCCCCGGCAT
TCCCTACAGAACCCTGGAGCCCCTGGAGACGGAAGGAGCCACCCGCTCCA
TGCTCAGCAGCTTTTTGCCTCCTGTTCCCAGCCTGCCCAGTGACCCCCCA
GAGCACTTCCCTCTGCGCAAGACAGTCTCTGAGCCCAACCTGAAGCTGCG
CTATAAGCCCAAGAAGTCCCTGGAGCGGAGGAAGAATCCACTGCTCCGAA
AGGAGAGTGCGCCCCCCAGCCTCCGGCGGCGGCCCGCAGAGACCCTCGGA
GACTCCTCCCCAAGTAGTAGCAGCACGCCCGCATCAGGGTGCAGCTCCCC
CAATGACAGCGAGCACGGCCCCAATCCCATCCTGGGCGACAGTGACCGCA
GGACCCATCCGACTCTGGGCCCTCGGGGGCCAATCCTGGGGAGCCCCCAC
ACTCCCCTCTTCCTGCCCCATGGCTTGGAGCCCGAGGCTGGGGGCACCTT
GCCCTCTCGCCTGCAGCCCATTCTCCTCCTGGACCCCTCAGGCTCTCATG
CCCCGCTGCTGACTGTGCCCGGGCTTGGGCCCTTGCCCTTCCACTTTGCC
CAGTCCTTAATGACCACCGAGCGGCTCTCTGGGTCAGGCCTCCACTGGCC
ACTGAGCCGGACTCGCTCAGAGCCCCTGCCCCCCAGTGCCACCGCTCCCC
CACCGCCGGGCCCCATGCAGCCCCGCCTGGAGCAGCTCAAAACTCACGTC
CAGGTGATCAAGAGGTCAGCCAAGCCGAGTGAGAAGCCCCGGCTGCGGCA
GATACCCTCGGCTGAAGACCTGGAGACAGATGGCGGGGGACCGGGCCAGG
TGGTGGACGATGGCCTGGAGCACAGGGAGCTGGGCCATGGGCAGCCTGAG
GCCAGAGGCCCCGCTCCTCTCCAGCAGCACCCTCAGGTGTTGCTCTGGGA
ACAGCAGCGACTGGCTGGGCGGCTCCCCCGGGGCAGCACCGGGGACACTG
TGCTGCTTCCTCTGGCCCAGGGTGGGCACCGGCCTCTGTCCCGGGCTCAG
TCTTCCCCAGCCGCACCTGCCTCACTGTCAGCCCCAGAGCCTGCCAGCCA
GGCCCGAGTCCTCTCCAGCTCAGAGACCCCTGCCAGGACCCTGCCCTTCA
CCACAGGGCTGATCTATGACTCGGTCATGCTGAAGCACCAGTGCTCCTGC
GGTGACAACAGCAGGCACCCGGAGCACGCCGGCCGCATCCAGAGCATCTG
GTCCCGGCTGCAGGAGCGGGGGCTCCGGAGCCAGTGTGAGTGTCTCCGAG
GCCGGAAGGCCTCCCTGGAAGAGCTGCAGTCGGTCCACTCTGAGCGGCAC
GTGCTCCTCTACGGCACCAACCCGCTCAGCCGCCTCAAACTGGACAACGG
GAAGCTGGCAGGGCTCCTGGCACAGCGGATGTTTGTGATGCTGCCCTGTG
GTGGGGTTGGGGTGGACACTGACACCATCTGGAATGAGCTTCATTCCTCC
AATGCAGCCCGCTGGGCCGCTGGCAGTGTCACTGACCTCGCCTTCAAAGT
GGCTTCTCGTGAGCTAAAGAATGGTTTCGCTGTGGTGCGGCCCCCAGGAC
ACCATGCAGATCATTCAACAGCCATGGGCTTCTGCTTCTTCAACTCAGTG
GCCATCGCCTGCCGGCAGCTGCAACAGCAGAGCAAGGCCAGCAAGATCCT
CATTGTAGACTGGGACGTGCACCATGGCAACGGCACCCAGCAAACCTTCT
ACCAAGACCCCAGTGTGCTCTACATCTCCCTGCATCGCCATGACGACGGC
AACTTCTTCCCGGGGAGTGGGGCTGTGGATGAGGTAGGGGCTGGCAGCGG
TGAGGGCTTCAATGTCAATGTGGCCTGGGCTGGAGGTCTGGACCCCCCCA
TGGGGGATCCTGAGTACCTGGCTGCTTTCAGGATAGTCGTGATGCCCATC
GCCCGAGAGTTCTCTCCAGACCTAGTCCTGGTGTCTGCTGGATTTGATGC
TGCTGAGGGTCACCCGGCCCCACTGGGTGGCTACCATGTTTCTGCCAAAT
GTTTTGGATACATGACGCAGCAACTGATGAACCTGGCAGGAGGCGCAGTG
GTGCTGGCCTTGGAGGGTGGCCATGACCTCACAGCCATCTGTGACGCCTC
TGAGGCCTGTGTGGCTGCTCTTCTGGGTAACAGGGTGGATCCCCTTTCAG
AAGAAGGCTGGAAACAGAAACCCAACCTCAATGCCATCCGCTCTCTGGAG
GCCGTGATCCGGGTGCACAGTAAATACTGGGGCTGCATGCAGCGCCTGGC
CTCCTGTCCAGACTCCTGGGTGCCTAGAGTGCCAGGGGCTGACAAAGAAG
AAGTGGAGGCAGTGACCGCACTGGCGTCCCTCTCTGTGGGCATCCTGGCT
GAAGATAGGCCCTCGGAGCAGCTGGTGGAGGAGGAAGAACCTATGAATCT
CTAAGGCTCTGGAACCATCTGCCCGCCCACCATGCCCTTGGGACCTGGTT
CTCTTCTAACCCCTGGCAATAGCCCCCATTCCTGGGTCTTTAGAGATCCT
GTGGGCAAGTAGTTGGAACCAGAGAACAGCCTGCCTGCTTTGACAGTTAT
CCCAGGGAGCGTGAGAAAATCCCTGGGTCTAGAATGGGAACTGGAGAGGA
CCCTGAGAGGAGACGGGCTGGGCGGCGACCCCCACAGGGCTCTCGAGAAC
AGATTCTCCCCTCCAGTATGGGCCCTGGCTGTGGCCCCCATTCCTCAGGA
CTGCACAGAGGAGGACTGGCTCCGGCTCCGTCGGGCTCACCCTTAACCAC
TATTCCTGGCTCTGCAAACCCCAGACTTTGCACACAGCCCCAGGCTCCAC
ACAGAAATGTGAACTTGGCCTCAGACAGGCTGGCCCTTCCTAGGCTCTAG
GGGCTAGGGGGGAGTGGGGAGCCAAGAGGTCCCATATTCCTGAGTGCAGG
GGTAGTCCCTCTCACCTGCTTCCTCAGACGACTCTGGAAGCTTCCCTCTA
CCACCGGGCACTGAGACGAAGCTCCCTGACAGCCGAGACTGGCAGCCCTC
CATCTGGTCCGTACCCTCGCCAGAGGCCCCCCTACATCAACCTCCTGGCG
ATGCCCTGGTGGAGCAGATGGGTGCTCTGGGAGTCCTGTGCTTCCTGATC
CAATGGTGCCAAACCCTTCATCTCCCCCAGAAGCGCAGCATACCCCTGGG
ACCCCTCGGCCACTGCCCACTCGGGGAGCCTTCTCTGTTTCTGGGGCCTC
CCCCACCATAGCTCTGATTCCCACCCCACATAGGAATAGCCTGACTGAGG
GGGAAGGGGTGGGAGAGAAGATACAGACATGGAGGAGGGGAGGCTGCTCT
GGCAAAGTCTTCAAGGCTTTTGGGGGTCCAGGCCTGGGGTCAAGAAGGAA
AATGTGTGTGAGCATGTGTGTGAGTGAGGCGTGTGTGTGAGCGTGTGTGT
GAGTGAGGCGTGTGTGTGTGTCTTTCCTAGGACCCACCATACCCTGTGTA
TGTATGCATGTTTTTGTAAAAAGGAAGAAAATGGAAAAAAATCTGAACAA
TAAATGTTTTATTTGCTTTAAAAAAAAAAAAAAAAA

SEQ ID NO:102

>gi|7662279|ref|NM_014707.1|Homo sapiens histone
deacetylase 9 (HDAC9-PENDING), transcript variant
3, mRNA
GGGGAAGAGAGGCACAGACACAGATAGGAGAAGGGCACCGGCTGGAGCCA
CTTGCAGGACTGAGGGTTTTTGCAACAAAACCCTAGCAGCCTGAAGAACT
CTAAGCCAGATGGGGTGGCTGGACGAGAGCAGCTCTTGGCTCAGCAAAGA
ATGCACAGTATGATCAGCTCAGTGGATGTGAAGTCAGAAGTTCCTGTGGG
CCTGGAGCCCATCTCACCTTTAGACCTAAGGACAGACCTCAGGATGATGA
TGCCCGTGGTGGACCCTGTTGTCCGTGAGAAGCAATTGCAGCAGGAATTA
CTTCTTATCCAGCAGCAGCAACAAATCCAGAAGCAGCTTCTGATAGCAGA
GTTTCAGAAACAGCATGAGAACTTGACACGGCAGCACCAGGCTCAGCTTC
AGGAGCATATCAAGGAACTTCTAGCCATAAAACAGCAACAAGAACTCCTA
GAAAAGGAGCAGAAACTGGAGCAGCAGAGGCAAGAACAGGAAGTAGAGAG
GCATCGCAGAGAACAGCAGCTTCCTCCTCTCAGAGGCAAAGATAGAGGAC
GAGAAAGGGCAGTGGCAAGTACAGAAGTAAAGCAGAAGCTTCAAGAGTTC
CTACTGAGTAAATCAGCAACGAAAGACACTCCAACTAATGGAAAAAATCA
TTCCGTGAGCCGCCATCCCAAGCTCTGGTACACGGCTGCCCACCACACAT
CATTGGATCAAAGCTCTCCACCCCTTAGTGGAACATCTCCATCCTACAAG
TACACATTACCAGGAGCACAAGATGCAAAGGATGATTTCCCCCTTCGAAA
AACTGCCTCTGAGCCCAACTTGAAGGTGCGGTCCAGGTTAAAACAGAAAG
TGGCAGAGAGGAGAAGCAGCCCCTTACTCAGGCGGAAGGATGGAAATGTT
GTCACTTCATTCAAGAAGCGAATGTTTGAGGTGACAGAATCCTCAGTCAG
TAGCAGTTCTCCAGGCTCTGGTCCCAGTTCACCAAACAATGGGCCAACTG
GAAGTGTTACTGAAAATGAGACTTCGGTTTTGCCCCCTACCCCTCATGCC
GAGCAAATGGTTTCACAGCAACGCATTCTAATTCATGAAGATTCCATGAA
CCTGCTAAGTCTTTATACCTCTCCTTCTTTGCCCAACATTACCTTGGGGC
TTCCCGCAGTGCCATCCCAGCTCAATGCTTCGAATTCACTCAAAGAAAAG
CAGAAGTGTGAGACGCAGACGCTTAGGCAAGGTGTTCCTCTGCCTGGGCA
GTATGGAGGCAGCATCCCGGCATCTTCCAGCCACCCTCATGTTACTTTAG
AGGGAAAGCCACCCAACAGCAGCCACCAGGCTCTCCTGCAGCATTTATTA
TTGAAAGAACAAATGCGACAGCAAAAGCTTCTTGTAGCTGGTGGAGTTCC
CTTACATCCTCAGTCTCCCTTGGCAACAAAAGAGAGAATTTCACCTGGCA
TTAGAGGTACCCACAAATTGCCCCGTCACAGACCCCTGAACCGAACCCAG
TCTGCACCTTTGCCTCAGAGCACGTTGGCTCAGCTGGTCATTCAACAGCA
ACACCAGCAATTCTTGGAGAAGCAGAAGCAATACCAGCAGCAGATCCACA
TGAACAAACTGCTTTCGAAATCTATTGAACAACTGAAGCAACCAGGCAGT
CACCTTGAGGAAGCAGAGGAAGAGCTTCAGGGGGACCAGGCGATGCAGGA
AGACAGAGCGCCCTCTAGTGGCAACAGCACTAGGAGCGACAGCAGTGCTT
GTGTGGATGACACACTGGGACAAGTTGGGGCTGTGAAGGTCAAGGAGGAA
CCAGTGGACAGTGATGAAGATGCTCAGATCCAGGAAATGGAATCTGGGGA
GCAGGCTGCTTTTATGCAACAGGTAATAGGCAAAGATTTAGCTCCAGGAT
TTGTAATTAAAGTCATTATCTGAACATGAAATGCATTGCAGGTTTGGTAA
ATGGATATGATTTCCTATCAGTTTATATTTCTCTATGATTTGAGTTCAGT
GTTTAAGGATTCTACCTAATGCAGATATATGTATATATCTATATAGAGGT
CTTTCTATATACTGATCTCTATATAGATATCAATGTTTCATTGAAAATCC
ACTGGTAAGGAAATACCTGTTATACTAAAATTATGATACATAATATCTGA
GCAGTTAATAGGCTTTAAATTTATCCCAAAGCCTGCTACACCAATTACTT
CTAAAGAAAACAAATTCACTGTTATTTTGAGTTTATGTGTTGAGATCAGT
GACTGCTGGATAGTCTCCCAGTCTGATCAATGAAGCATTCGATTAGTTTT
TGATTTTTTGCAACATCTAGAATTTAATTTTCACATCACTGTACATAATG
TATCATACTATAGTCTTGAACACTGTTAAAGGTAGTCTGCCCCTTCCTTC
CTCTCTCTTTTTTTAGTTAAGTAGAAATGTTCTGGTCACCATGCCAGTAG
TCCTAGGTTATTGTGTAGGTTGCAATTGAACATATTAGGAATACAGGTGG
TTTTAAATATATAGATGCAAATTGCAGCACTACTTTAAATATTAGATTAT
GTCTCACATAGCACTGCTCATTTTACTTTTATTTTGTGTAATTTGATGAC
ACTGTCTATCAAAAAAGAGCAAATGAAGCAGATGCAAATGTTAGTGAGAA
GTAATGTGCAGCATTATGGTCCAATCAGATACAATATTGTGTCTACAATT
GCAAAAAACACAGTAACAGGATGAATATTATCTGATATCAAGTCAAAATC
AGTTTGAAAAGAAGGTGTATCATATTTTATATTGTCACTAGAATCTCTTA
AGTATAATTCCATAATGACATGGGCATATACCGTAACATTCTGGCAAATA
ACAATTAGAAAAGATAGGTTTAACAAAAAAATTTACTTGTATATAATGCA
CCTTCAGGAGGACTATGTCCTTTGATGCTATAAAATACAAACAACTTTGA
AGGCAACAGAAGACACTGTTTATTCAAGTCAGTTCTTTGTCAGGTTCCTG
CTGTTCTCCTACAGAAAAGTGATTCTGTGAGGGTGAACAGGAAATGCCTT
GTGGAAACAGGAAGTCCAAGTGATTCATGTACTGAGGAATGTAGGAAAAA
AAATCTGAGGATAGTGCTTTACTCTTTCTGTTTTTAAAGGGCACTCTATG
AATTGATTTATTGTCTAAGAAAATAACACCACAAGTAGGGAAATTGTTAC
GGAAGCTTTTCACTGGAACATTTCCTTCATATTCCCTTTTGATATGTTTA
CCTTGTTTTATAGGTTTACTTTTGTTAAGCTAGTTAAAGGTTCGTTGTAT
TAAGACCCCTTTAATATGGATAATCCAAATTGACCTAGAATCTTTGTGAG
GTTTTTTCTATTAAAATATTTATATTTCTAAATCCGAGGTATTTCAAGGT
GTAGTATCCTATTTCAAAGGAGATATAGCAGTTTTGCCAAATGTAGACAT
TGTTCAACTGTATGTTATTGGCACGTGTTGTTTACATTTTGCTGTGACAT
TTAAAAATATTTCTTTAAAAATGTTACTGCTAAAGATACATTATCCTTTT
TTAAAAAGTCTCCATTCAAATTAAATTAACATAACTAGAAGTTAGAAAGT
TTAAAAGTTTTCCACATAATGAAAGTCCTTCTGATAATTTGACAAATAGC
TATAATAGGAACACTCCCTATCACCAACATATTTTGGTTAGTATATTCCT
TCATATTAAAATGACTTTTTGTCAGTTGTTTTGCATTAAAAATATGGCAT
GCCTAAGATAAAATTGTATATTTTTTCCATCTCATAAATATTCATTTTCT
TCAAAGTCTTTTTTCAATCTCATAAAAAAGGGATAGTGCATCTTTTAAAA
TACATTTTATTTGGGGAGGAACATGTGGCTGAGCAGACTTTTGTATAATA
TTACTTCAAAGATATGTAATCACAAACAAAAAAAACTATTTTTTATAATG
TCATTTGAGAGAGTTTCATCAGTACAGTTGGTGGACGTTAATTGTTTGAA
TTTGATAGTCTTTGAATTTAATCAAGAAACTACCTGGAACCAGTGAAAAG
GAAAGCTGGACTTAAATAATCTTAGAATTAATTGATAAATGTCTCTTTTA
AAATCTACTGTATTTATTATAATTTACACCCTTGAAGGTGATCTCTTGTT
TTGTGTTGTAAATATATTGTTTGTATGTTTCCCTTCTTGCCTTCTGTTAT
AAGTCTCTTCCTTTCTCAAATAAAGTTTTTTTTAAAAG
HDAC8

SEQ ID NO:103

>gi|8923768|ref|NM_018486.1|Homo sapiens histone
deacetylase 8 (HDAC8), mRNA
GCCAGATCTGGAAGGTGGCTGCGGAACGGTTTTAAGCGGAAGATGGAGGA
GCCGGAGGAACCGGCGGACAGTGGGCAGTCGCTGGTCCCGGTTTATATCT
ATAGTCCCGAGTATGTCAGTATGTGTGACTCCCTGGCCAAGATCCCCAAA
CGGGCCAGTATGGTGCATTCTTTGATTGAAGCATATGCACTGCATAAGCA
GATGAGGATAGTTAAGCCTAAAGTGGCCTCCATGGAGGAGATGGCCACCT
TCCACACTGATGCTTATCTGCAGCATCTCCAGAAGGTCAGCCAAGAGGGC
GATGATGATCATCCGGACTCCATAGAATATGGGCTAGGTTATGACTGCCC
AGCCACTGAAGGGATATTTGACTATGCAGCAGCTATAGGAGGGGCTACGA
TCACAGCTGCCCAATGCCTGATTGACGGAATGTGCAAAGTAGCAATTAAC
TGGTCTGGAGGGTGGCATCATGCAAAGAAAGATGAAGCATCTGGTTTTTG
TTATCTCAATGATGCTGTCCTGGGAATATTACGATTGCGACGGAAATTTG
AGCGTATTCTCTACGTGGATTTGGATCTGCACCATGGAGATGGTGTAGAA
GACGCATTCAGTTTCACCTCCAAAGTCATGACCGTGTCCCTGCACAAATT
CTCCCCAGGATTTTTCCCAGGAACAGGTGACGTGTCTGATGTTGGCCTAG
GGAAGGGACGGTACTACAGTGTAAATGTGCCCATTCAGGATGGCATACAA
GATGAAAAATATTACCAGATCTGTGAAAGTGTACTAAAGGAAGTATACCA
AGCCTTTAATCCCAAAGCAGTGGTCTTACAGCTGGGAGCTGACACAATAG
CTGGGGATCCCATGTGCTCCTTTAACATGACTCCAGTGGGAATTGGCAAG
TGTCTTAAGTACATCCTTCAATGGCAGTTGGCAACACTCATTTTGGGAGG
AGGAGGCTATAACCTTGCCAACACGGCTCGATGCTGGACATACTTGACCG
GGGTCATCCTAGGGAAAACACTATCCTCTGAGATCCCAGATCATGAGTTT
TTCACAGCATATGGTCCTGATTATGTGCTGGAAATCACGCCAAGCTGCCG
GCCAGACCGCAATGAGCCCCACCGAATCCAACAAATCCTCAACTACATCA
AAGGGAATCTGAAGCATGTGGTCTAGTTGACAGAAAGAGATCAGGTTTCC
AGAGCTGAGGAGTGGTGCCTATAATGAAGACAGCGTGTTTATGCAAGCAG
TTTGTGGAATTTGTGACTGCAGGGAAAATTTGAAAGAAATTACTTCCTGA
AAATTTCCAAGGGGCATCAAGTGGCAGCTGGCTTCCTGGGGTGAAGAGGC
AGGCACCCCAGAGTCCTCAACTGGACCTAGGGGAAGAAGGAGATATCCCA
CATTTAAAGTTCTTATTTAAAAAAACACACACACACAAATGAAATTTTTA
ATCTTTGAAAATTATTTTTAAGCGAATTGGGGAGGGGAGTATTTTAATCA
TCTTAAATGAAACAGATCAGAAGCTGGATGAGAGCAGTCACCAGTTTGTA
GGGCAGGAGGCAGCTGAGAGGCAGGGTTTGGGCCTCAGGACCATCCAGGT
GGAGCCCTGGGAGAGAGGGTACTGATCAGCAGACTGGGAGGTGGGGAGAA
GTCCGCTGGTGTTGTTTTAGTGTTATATATCTTTGGTTTTTTTAATAAAA
TCTTTGAAAACCTAAAAAAAAAAAAAAAAAAAAAAAAAAAAAA
hSIRT2

SEQ ID NO:104

>gi|13775599|ref|NM_012237.2|Homo sapiens sirtuin
silent mating type information regulation 2
homolog 2 (S. cerevisiae) (SIRT2), transcript
variant 1, mRNA
GTGTTGTACGAAAGCGCGTCTGCGGCCGCAATGTCTGCTGAGAGTTGTAG
TTCTGTGCCCTATCACGGCCACTCCCATTTCTGGTGCCGTCACGGGACAG
AGCAGTCGGTGACAGGACAGAGCAGTCGGTGACGGGACACAGTGGTTGGT
GACGGGACAGAGCGGTCGGTGACAGCCTCAAGGGCTTCAGCACCGCGCCC
ATGGCAGAGCCAGACCCCTCTCACCCTCTGGAGACCCAGGCAGGGAAGGT
GCAGGAGGCTCAGGACTCAGATTCAGACTCTGAGGGAGGAGCCGCTGGTG
GAGAAGCAGACATGGACTTCCTGCGGAACTTATTCTCCCAGACGCTCAGC
CTGGGCAGCCAGAAGGAGCGTCTGCTGGACGAGCTGACCTTGGAAGGGGT
GGCCCGGTACATGCAGAGCGAACGCTGTCGCAGAGTCATCTGTTTGGTGG
GAGCTGGAATCTCCACATCCGCAGGCATCCCCGACTTTCGCTCTCCATCC
ACCGGCCTCTATGACAACCTAGAGAAGTACCATCTTCCCTACCCAGAGGC
CATCTTTGAGATCAGCTATTTCAAGAAACATCCGGAACCCTTCTTCGCCC
TCGCCAAGGAACTCTATCCTGGGCAGTTCAAGCCAACCATCTGTCACTAC
TTCATGCGCCTGCTGAAGGACAAGGGGCTACTCCTGCGCTGCTACACGCA
GAACATAGATACCCTGGAGCGAATAGCCGGGCTGGAACAGGAGGACTTGG
TGGAGGCGCACGGCACCTTCTACACATCACACTGCGTCAGCGCCAGCTGC
CGGCACGAATACCCGCTAAGCTGGATGAAAGAGAAGATCTTCTCTGAGGT
GACGCCCAAGTGTGAAGACTGTCAGAGCCTGGTGAAGCCTGATATCGTCT
TTTTTGGTGAGAGCCTCCCAGCGCGTTTCTTCTCCTGTATGCAGTCAGAC
TTCCTGAAGGTGGACCTCCTCCTGGTCATGGGTACCTCCTTGCAGGTGCA
GCCCTTTGCCTCCCTCATCAGCAAGGCACCCCTCTCCACCCCTCGCCTGC
TCATCAACAAGGAGAAAGCTGGCCAGTCGGACCCTTTCCTGGGGATGATT
ATGGGCCTCGGAGGAGGCATGGACTTTGACTCCAAGAAGGCCTACAGGGA
CGTGGCCTGGCTGGGTGAATGCGACCAGGGCTGCCTGGCCCTTGCTGAGC
TCCTTGGATGGAAGAAGGAGCTGGAGGACCTTGTCCGGAGGGAGCACGCC
AGCATAGATGCCCAGTCGGGGGCGGGGGTCCCCAACCCCAGCACTTCAGC
TTCCCCCAAGAAGTCCCCGCCACCTGCCAAGGACGAGGCCAGGACAACAG
AGAGGGAGAAACCCCAGTGACAGCTGCATCTCCCAGGCGGGATGCCGAGC
TCCTCAGGGACAGCTGAGCCCCAACCGGGCCTGGCCCCCTCTTAACCAGC
AGTTCTTGTCTGGGGAGCTCAGAACATCCCCCAATCTCTTACAGCTCCCT
CCCCAAAACTGGGGTCCCAGCAACCCTGGCCCCCAACCCCAGCAAATCTC
TAACACCTCCTAGAGGCCAAGGCTTAAACAGGCATCTCTACCAGCCCCAC
TGTCTCTAACCACTCCTGGGCTAAGGAGTAACCTCCCTCATCTCTAACTG
CCCCCACGGGGCCAGGGCTACCCCAGAACTTTTAACTCTTCCAGGACAGG
GAGCTTCGGGCCCCCACTCTGTCTCCTGCCCCCGGGGGCCTGTGGCTAAG
TAAACCATACCTAACCTACCCCAGTGTGGGTGTGGGCCTCTGAATATAAC
CCACACCCAGCGTAGGGGGAGTCTGAGCCGGGAGGGCTCCCGAGTCTCTG
CCTTCAGCTCCCAAAGTGGGTGGTGGGCCCCCTTCACGTGGGACCCACTT
CCCATGCTGGATGGGCAGAAGACATTGCTTATTGGAGACAAATTAAAAAC
AAAAACAACTAAC

SEQ ID NO:105

>gi|13775601|ref|NM_030593.1|Homo sapiens sirtuin
silent mating type information regulation 2
homolog 2 (S. cerevisiae) (SIRT2), transcript
variant 2, mRNA
CGAAAGCGCGTCTGCGGCCGCAATGTCTGCTGAGAGTTGTAGTTCTGTGC
CCTATCACGGCCACTCCCATTTCTGGTGCCGTCACGGGACAGAGCAGTCG
GTGACAGGACAGAGCAGTCGGTGACGGGACACAGTGGTTGGTGACGGGAC
AGAGCGGTCGGTGACAGCCTCAAGGGCTTCAGCACCGCGCCCATGGCAGA
GCCAGACCGACTCAGATTCAGACTCTGAGGGAGGAGCCGCTGGTGGAGAA
GCAGACATGGACTTCCTGCGGAACTTATTCTCCCAGACGCTCAGCCTGGG
CAGCCAGAAGGAGCGTCTGCTGGACGAGCTGACCTTGGAAGGGGTGGCCC
GGTACATGCAGAGCGAACGCTGTCGCAGAGTCATCTGTTTGGTGGGAGCT
GGAATCTCCACATCCGCAGGCATCCCCGACTTTCGCTCTCCATCCACCGG
CCTCTATGACAACCTAGAGAAGTACCATCTTCCCTACCCAGAGGCCATCT
TTGAGATCAGCTATTTCAAGAAACATCCGGAACCCTTCTTCGCCCTCGCC
AAGGAACTCTATCCTGGGCAGTTCAAGCCAACCATCTGTCACTACTTCAT
GCGCCTGCTGAAGGACAAGGGGCTACTCCTGCGCTGCTACACGCAGAACA
TAGATACCCTGGAGCGAATAGCCGGGCTGGAACAGGAGGACTTGGTGGAG
GCGCACGGCACCTTCTACACATCACACTGCGTCAGCGCCAGCTGCCGGCA
CGAATACCCGCTAAGCTGGATGAAAGAGAAGATCTTCTCTGAGGTGACGC
CCAAGTGTGAAGACTGTCAGAGCCTGGTGAAGCCTGATATCGTCTTTTTT
GGTGAGAGCCTCCCAGCGCGTTTCTTCTCCTGTATGCAGTCAGACTTCCT
GAAGGTGGACCTCCTCCTGGTCATGGGTACCTCCTTGCAGGTGCAGCCCT
TTGCCTCCCTCATCAGCAAGGCACCCCTCTCCACCCCTCGCCTGCTCATC
AACAAGGAGAAAGCTGGCCAGTCGGACCCTTTCCTGGGGATGATTATGGG
CCTCGGAGGAGGCATGGACTTTGACTCCAAGAAGGCCTACAGGGACGTGG
CCTGGCTGGGTGAATGCGACCAGGGCTGCCTGGCCCTTGCTGAGCTCCTT
GGATGGAAGAAGGAGCTGGAGGACCTTGTCCGGAGGGAGCACGCCAGCAT
AGATGCCCAGTCGGGGGCGGGGGTCCCCAACCCCAGCACTTCAGCTTCCC
CCAAGAAGTCCCCGCCACCTGCCAAGGACGAGGCCAGGACAACAGAGAGG
GAGAAACCCCAGTGACAGCTGCATCTCCCAGGCGGGATGCCGAGCTCCTC
AGGGACAGCTGAGCCCCAACCGGGCCTGGCCCCCTCTTAACCAGCAGTTC
TTGTCTGGGGAGCTCAGAACATCCCCCAATCTCTTACAGCTCCCTCCCCA
AAACTGGGGTCCCAGCAACCCTGGCCCCCAACCCCAGCAAATCTCTAACA
CCTCCTAGAGGCCAAGGCTTAAACAGGCATCTCTACCAGCCCCACTGTCT
CTAACCACTCCTGGGCTAAGGAGTAACCTCCCTCATCTCTAACTGCCCCC
ACGGGGCCAGGGCTACCCCAGAACTTTTAACTCTTCCAGGACAGGGAGCT
TCGGGCCCCCACTCTGTCTCCTGCCCCCGGGGGCCTGTGGCTAAGTAAAC
CATACCTAACCTACCCCAGTGTGGGTGTGGGCCTCTGAATATAACCCACA
CCCAGCGTAGGGGGAGTCTGAGCCGGGAGGGCTCCCGAGTCTCTGCCTTC
AGCTCCCAAAGTGGGTGGTGGGCCCCCTTCACGTGGGACCCACTTCCCAT
GCTGGATGGGCAGAAGACATTGCTTATTGGAGACAAATTAAAAACAAAAA
CAACTAACAAAAAAAAAAAAAAAAAAAAAAA
hSIRT3

SEQ ID NO:106

>gi|13775603|ref|NM_012239.3|Homo sapiens sirtuin
silent mating type information regulation 2
homolog 3 (S. cerevisiae) (SIRT3), mRNA
GGCGCCGGGGGCGGGGGTGGGAGGCGGAGGCGGGGCCGGGGCGCCGCGGG
CGGGGCGCCGGGGGCGGGGCGAGTCCGGAGGACTCCTCGGACTGCGCGGA
ACATGGCGTTCTGGGGTTGGCGCGCCGCGGCAGCCCTCCGGCTGTGGGGC
CGGGTAGTTGAACGGGTCGAGGCCGGGGGAGGCGTGGGGCCGTTTCAGGC
CTGCGGCTGTCGGCTGGTGCTTGGCGGCAGGGACGATGTGAGTGCGGGGC
TGAGAGGCAGCCATGGGGCCCGCGGTGAGCCCTTGGACCCGGCGCGCCCC
TTGCAGAGGCCTCCCAGACCCGAGGTGCCCAGGGCATTCCGGAGGCAGCC
GAGGGCAGCAGCTCCCAGTTTCTTCTTTTCGAGTATTAAAGGTGGAAGAA
GGTCCATATCTTTTTCTGTGGGTGCTTCAAGTGTTGTTGGAAGTGGAGGC
AGCAGTGACAAGGGGAAGCTTTCCCTGCAGGATGTAGCTGAGCTGATTCG
GGCCAGAGCCTGCCAGAGGGTGGTGGTCATGGTGGGGGCCGGCATCAGCA
CACCCAGTGGCATTCCAGACTTCAGATCGCCGGGGAGTGGCCTGTACAGC
AACCTCCAGCAGTACGATCTCCCGTACCCCGAGGCCATTTTTGAACTCCC
ATTCTTCTTTCACAACCCCAAGCCCTTTTTCACTTTGGCCAAGGAGCTGT
ACCCTGGAAACTACAAGCCCAACGTCACTCACTACTTTCTCCGGCTGCTT
CATGACAAGGGGCTGCTTCTGCGGCTCTACACGCAGAACATCGATGGGCT
TGAGAGAGTGTCGGGCATCCCTGCCTCAAAGCTGGTTGAAGCTCATGGAA
CCTTTGCCTCTGCCACCTGCACAGTCTGCCAAAGACCCTTCCCAGGGGAG
GACATTCGGGCTGACGTGATGGCAGACAGGGTTCCCCGCTGCCCGGTCTG
CACCGGCGTTGTGAAGCCCGACATTGTGTTCTTTGGGGAGCCGCTGCCCC
AGAGGTTCTTGCTGCATGTGGTTGATTTCCCCATGGCAGATCTGCTGCTC
ATCCTTGGGACCTCCCTGGAGGTGGAGCCTTTTGCCAGCTTGACCGAGGC
CGTGCGGAGCTCAGTTCCCCGACTGCTCATCAACCGGGACTTGGTGGGGC
CCTTGGCTTGGCATCCTCGCAGCAGGGACGTGGCCCAGCTGGGGGACGTG
GTTCACGGCGTGGAAAGCCTAGTGGAGCTTCTGGGCTGGACAGAAGAGAT
GCGGGACCTTGTGCAGCGGGAAACTGGGAAGCTTGATGGACCAGACAAAT
AGGATGATGGCTGCCCCCACACAATAAATGGTAACATAGGAGACATCCAC
ATCCCAATTCTGACAAGACCTCATGCCTGAAGACAGCTTGGGCAGGTGAA
ACCAGAATATGTGAACTGAGTGGACACCCGAGGCTGCCACTGGAATGTCT
TCTCAGGCCATGAGCTGCAGTGACTGGTAGGGCTGTGTTTACAGTCAGGG
CCACCCCGTCACATATACAAAGGAGCTGCCTGCCTGTTTGCTGTGTTGAA
CTCTTCACTCTGCTGAAGCTCCTAATGGAAAAAGCTTTCTTCTGACTGTG
ACCCTCTTGAACTGAATCAGACCAACTGGAATCCCAGACCGAGTCTGCTT
TCTGTGCCTAGTTGAACGGCAAGCTCGGCATCTGTTGGTTACAAGATCCA
GACTTGGGCCGAGCGGTCCCCAGCCCTCTTCATGTTCCGAAGTGTAGTCT
TGAGGCCCTGGTGCCGCACTTCTAGCATGTTGGTCTCCTTTAGTGGGGCT
ATTTTTAATGAGAGAAAATCTGTTCTTTCCAGCATGAAATACATTTAGTC
TCCTCAAAGGGAAAAAAAAAAAAAAAAAAAAAAAAAAAAAAAAAAAAAAA
AAAAAAAAAAAAAAAAAAAAAAAAAAA
hSIRT4

SEQ ID NO:107

>gi|6912661|ref|NM_012240.1|Homo sapiens sirtuin
silent mating type information regulation 2
homolog 4 (S. cerevisiae) (SIRT4), mRNA
GTCCGTAGAGCTGTGAGAGAATGAAGATGAGCTTTGCGTTGACTTTCAGG
TCAGCAAAAGGCCGTTGGATCGCAAACCCCAGCCAGCCGTGCTCGAAAGC
CTCCATTGGGTTATTTGTGCCAGCAAGTCCTCCTCTGGACCCTGAGAAGG
TCAAAGAGTTACAGCGCTTCATCACCCTTTCCAAGAGACTCCTTGTGATG
ACTGGGGCAGGAATCTCCACCGAATCGGGGATACCAGACTACAGGTCAGA
AAAAGTGGGGCTTTATGCCCGCACTGACCGCAGGCCCATCCAGCATGGTG
ATTTTGTCCGGAGTGCCCCAATCCGCCAGCGGTACTGGGCGAGAAACTTC
GTAGGCTGGCCTCAATTCTCCTCCCACCAGCCTAACCCTGCACACTGGGC
TTTGAGCACCTGGGAGAAACTCGGAAAGCTGTACTGGTTGGTGACCCAAA
ATGTGGATGCTTTGCACACCAAGGCGGGGAGTCGGCGCCTGACAGAGCTC
CACGGATGCATGGACAGGGTCCTGTGCTTGGATTGTGGGGAACAGACTCC
CCGGGGGGTGCTGCAAGAGCGTTTCCAAGTCCTGAACCCCACCTGGAGTG
CTGAGGCCCATGGCCTGGCTCCTGATGGTGACGTCTTTCTCTCAGAGGAG
CAAGTCCGGAGCTTTCAGGTCCCAACCTGCGTTCAATGTGGAGGCCATCT
GAAACCAGATGTCGTTTTCTTCGGGGACACAGTGAACCCTGACAAGGTTG
ATTTTGTGCACAAGCGTGTAAAAGAAGCCGACTCCCTCTTGGTGGTGGGA
TCATCCTTGCAGGTATACTCTGGTTACAGGTTTATCCTCACTGCCTGGGA
GAAGAAGCTCCCGATTGCAATACTGAACATTGGGCCCACACGGTCGGATG
ACTTGGCGTGTCTGAAACTGAATTCTCGTTGTGGAGAGTTGCTGCCTTTG
ATAGACCCATGCTGACCACAGCCTGATATTCCAGAACCTGGAACAGGGAC
TTTCACTTGAATCTTGCTGCTAAATGTAAATGCCTTCTCAAATGACAGAT
TCCAGTTCCCATTCAACAGAGTAGGGTGCACTGACAAAGTATAGAAGGTT
CTAGGTATCTTAATGTGTGGATATTCTTAATTAAAACTCATTTTTTTTAA
ATAAAAAATTGTTCAGCTTTAAAA
hSIRT5

SEQ ID NO:108

>gi|13787213|ref|NM_012241.2|Homo sapiens sirtuin
silent mating type information regulation 2
homolog 5 (S. cerevisiae) (SIRT5), transcript
variant 1, mRNA
CCGGAGCGCGGTCGGGACACAGCGCCTCTAGGAGAAAGCCTGGAAGGCGC
TCCGGGGGTACCCAGAGCTCTTAGCGGGCCGGCAGCATGTGCGGGGCCCA
AGTAAATGGAAATGTTTTCTAACATATAAAAACCTACAGAAGAAGAAAAT
AATTTTCTGGATCAAATTAGAAGTCTGTATTATATTGATGTCTCCAGATT
CAAATATATTAGAAAGCAGCCGTGGAGACAACCATCTTCATTTTGGGAGA
AATAACTAAAGCCCGCCTCAAGCATTAGAACTACAGACAAACCCTGATGC
GACCTCTCCAGATTGTCCCAAGTCGATTGATTTCCCAGCTATATTGTGGC
CTGAAGCCTCCAGCGTCCACACGAAACCAGATTTGCCTGAAAATGGCTCG
GCCAAGTTCAAGTATGGCAGATTTTCGAAAGTTTTTTGCAAAAGCAAAGC
ACATAGTCATCATCTCAGGAGCTGGTGTTAGTGCAGAAAGTGGTGTTCCG
ACCTTCAGAGGAGCTGGAGGTTATTGGAGAAAATGGCAAGCCCAGGACCT
GGCGACTCCCCTGGCCTTTGCCCACAACCCGTCCCGGGTGTGGGAGTTCT
ACCACTACCGGCGGGAGGTCATGGGGAGCAAGGAGCCCAACGCCGGGCAC
CGCGCCATAGCCGAGTGTGAGACCCGGCTGGGCAAGCAGGGCCGGCGAGT
CGTGGTCATCACCCAGAACATCGATGAGCTGCACCGCAAGGCTGGCACCA
AGAACCTTCTGGAGATCCATCGTAGCTTATTTAAAACTCGATGTACCTCT
TGTGGAGTTGTGGCTGAGAATTACAAGAGTCCAATTTGTCCAGCTTTATC
AGGAAAAGGTGCTCCAGAACCTGGAACTCAAGATGCCAGCATCCCAGTTG
AGAAACTTCCCCGGTGTGAAGAGGCAGGCTGCGGGGGCTTGCTGCGACCT
CACGTCGTGTGGTTTGGAGAAAACCTGGATCCTGCCATTCTGGAGGAGGT
TGACAGAGAGCTCGCCCACTGTGATTTATGTCTAGTGGTGGGCACTTCCT
CTGTGGTGTACCCAGCAGCCATGTTTGCCCCCCAGGTGGCTGCCAGGGGC
GTGCCAGTGGCTGAATTTAACACGGAGACCACCCCAGCTACGAACAGATT
CAGGTTTCATTTCCAGGGACCCTGTGGAACGACTCTTCCTGAAGCCCTTG
CCTGTCATGAAAATGAAACTGTTTCTTAAGTGTCCTGGGGAAGAAAGAAA
TTACAGTATATCTAAGAACTAGGCCACACGCAGAGGAGAAATGGTCTTAT
GGGTGGTGAGCTGAGTACTGAACAATCTAAAAATAGCCTCTGATTCCCTC
GCTGGAATCCAACCTGTTGATAAGTGATGGGGGTTTAGAAGTAGCAAAGA
GCACCCACATTCAAAAGTCACAGAACTGGAAAGTTAATTCATATTATTTG
GTTTGAACTGAAACGTGAGGTATCTTTGATGTGTATGGTTGGTTATTGGG
AGGGAAAAATTTTGTAAATTAGATTGTCTAAAAAAAATAGTTATTCTGAT
TATATTTTTGTTATCTGGGCAAAGTAGAAGTCAAGGGGTAAAAACCCTAC
TATTCTGATTTTTGCACAAGTTTTAGTGGAAAATAAAATCACACTCTACA
GTAAAAAAAAAAAAAAAAAA

SEQ ID NO:109

>gi|13787214|ref|NM_031244.1|Homo sapiens sirtuin
silent mating type information regulation 2
homolog 5 (S. cerevisiae) (SIRT5), transcript
variant 2, mRNA
ATTCGGGGGCGCGAGCTGCCCCAGTAAATGGAAATGTTTTCTAACATATA
AAAACCTACAGAAGAAGAAAATAATTTTCTGGATCAAATTAGAAGTCTGT
ATTATATTGATGTCTCCAGATTCAAATATATTAGAAAGCAGCCGTGGAGA
CAACCATCTTCATTTTGGGCGAAATAACTAAAGCCCGCCTCAAGCATTAG
AACTACAGACAAACCCTGATGCGACCTCTCCAGATTGTCCCAAGTCGATT
GATTTCCCAGCTATATTGTGGCCTGAAGCCTCCAGCGTCCACACGAAACC
AGATTTGCCTGAAAATGGCTCGGCCAAGTTCAAGTATGGCAGATTTTCGA
AAGTTTTTTGCAAAAGCAAAGCACATAGTCATCATCTCAGGAGCTGGTGT
TAGTGCAGAAAGTGGTGTTCCGACCTTCAGAGGAGCTGGAGGTTATTGGA
GAAAATGGCAAGCCCAGGACCTGGCGACTCCCCTGGCCTTTGCCCACAAC
CCGTCCCGGGTGTGGGAGTTCTACCACTACCGGCGGGAGGTCATGGGGAG
CAAGGAGCCCAACGCCGGGCACCGCGCCATAGCCGAGTGTGAGACCCGGC
TGGGCAAGCAGGGCCGGCGAGTCGTGGTCATCACCCAGAACATCGATGAG
CTGCACCGCAAGGCTGGCACCAAGAACCTTCTGGAGATCCATGGTAGCTT
ATTTAAAACTCGATGTACCTCTTGTGGAGTTGTGGCTGAGAATTACAAGA
GTCCAATTTGTCCAGCTTTATCAGGAAAAGGTGCTCCAGAACCTGGAACT
CAAGATGCCAGCATCCCAGTTGAGAAACTTCCCCGGTGTGAAGAGGCAGG
CTGCGGGGGCTTGCTGCGACCTCACGTCGTGTGGTTTGGAGAAAACCTGG
ATCCTGCCATTCTGGAGGAGGTTGACAGAGAGCTCGCCCACTGTGATTTA
TGTCTAGTGGTGGGCACTTCCTCTGTGGTGTACCCAGCAGCCATGTTTGC
CCCCCAGGTGGCTGCCAGGGGCGTGCCAGTGGCTGAATTTAACACGGAGA
CCACCCCAGCTACGAACAGATTCAGTCATTTGATCTCCATCTCATCTCTA
ATTATTATAAAGAATTAAAACAAGTCATCATTGTAGAAAAGCAAGAAAAT
GCAGATAGAGAAAAAGAAGAAAATAAAACTGGAGTATTTCCACAACCCAA
GTTTAGAGTTGGCCCCCACCTCCCATGCCATGGACTGAGCAGCAGGGGCC
CAGCATCCCTTGGATATGGTGGCTGTGTCTTCATGTGAAAGAAACTGAAC
TTGGTGGTTTTTCCTGCCAGTTCAGGAGAGATTCTTGGCATGTAATATAT
ATCACTGCTCAAGTCAAGCCTCCTAAAACCACAGACCTGTTTCAGCTGCT
ACTTCAGCCAAAATTCTTCAGCTTCATATTGTCTTGAAAACCTATGATTG
TCTCTAACAAACAGGCTACTTGCTAGTTAGAAATTCTTATCAATTTGGCA
AGCTACTTATCAACCAGACTGACCACAAGAACTGTCATCTCATCAATGAA
GGAGTAACTGATCAATGAAGCCAGCAATGCTTTTTTCTTGGCATCATCAA
AGCTGACATTTAGAAGAGATGCTGGTGATAGTCATCTCATCCTACTCAAT
TTTTCAAAGGCAGAAACCAACCCTGGAGCAATTGAGAGGACTGTTTAAAC
ACAGAGCTTAACAATGGCAGAATTGTATATCTCGTGCTTAACAGATTTTG
GTTGAACTTTACCCTAGGTCAGGGGTCAGCAAACTACTGCCTGTGGGCCA
AATTTGCCCACCACCTGTATCTGTAAATAAGGTTTCATTGGAACACAGCT
GTGGCCATATGTTTGTATATTGTGTGTGGCTGCTTTTGCATTAGGATGAC
AGAGGTGAATAGTTGCAACAGAGACTGGCTGGTCTGCAAAGCCTAAAATA
TGTCCTGTGTGGCCCTTTACAGAAAAAGTTTTCTAACCCCTGCTCTAGGT
TACGGAGAAAAAAAAATGGAATAATGTTCTCTGCTACTTTTAACCTGATT
TTCTTTGTACCTAAATAGGCAGCTAGAATGCTGCCTATATTTTAATAAGG
ATTTGGATCTCACAAGACACCTTAGGCCTACACAAGTTGTTCAGATTCTT
TGCCCCAGTTCTAATCTAGTGACAAAGGCATAGAATTCTCCTCCCACAGG
AATGTATTTCTATTTTCAAGGTGTTAATTAGTTCCAGTTTTGGTTTTGTC
GTTTTCCCCATGTCCGATGCTTATATTGGATGATTTCTGATAAACCTGAC
TATTCCAATAAACCCTAGGCATTTTTGAATTTAAAAAAAAAAAAAAAAAA
hSIRT6

SEQ ID NO:110

>gi|7706709|ref|NM_016539.1|Homo sapiens sirtuin
silent mating type information regulation 2
homolog 6 (S. cerevisiae) (SIRT6), mRNA
GCTTCCGGCGGAAGCGGCCTCAACAAGGGAAACTTTATTGTTCCCGTGGG
GCAGTCGAGGATGTCGGTGAATTACGCGGCGGGGCTGTCGCCGTACGCGG
ACAAGGGCAAGTGCGGCCTCCCGGAGATCTTCGACCCCCCGGAGGAGCTG
GAGCGGAAGGTGTGGGAACTGGCGAGGCTGGTCTGGCAGTCTTCCAGTGT
GGTGTTCCACACGGGTGCCGGCATCAGCACTGCCTCTGGCATCCCCGACT
TCAGGGGTCCCCACGGAGTCTGGACCATGGAGGAGCGAGGTCTGGCCCCC
AAGTTCGACACCACCTTTGAGAGCGCGCGGCCCACGCAGACCCACATGGC
GCTGGTGCAGCTGGAGCGCGTGGGCCTCCTCCGCTTCCTGGTCAGCCAGA
ACGTGGACGGGCTCCATGTGCGCTCAGGCTTCCCCAGGGACAAACTGGCA
GAGCTCCACGGGAACATGTTTGTGGAAGAATGTGCCAAGTGTAAGACGCA
GTACGTCCGAGACACAGTCGTGGGCACCATGGGCCTGAAGGCCACGGGCC
GGCTCTGCACCGTGGCTAAGGCAAGGGGGCTGCGAGCCTGCAGGGGAGAG
CTGAGGGACACCATCCTAGACTGGGAGGACTCCCTGCCCGACCGGGACCT
GGCACTCGCCGATGAGGCCAGCAGGAACGCCGACCTGTCCATCACGCTGG
GTACATCGCTGCAGATCCGGCCCAGCGGGAACCTGCCGCTGGCTACCAAG
CGCCGGGGAGGCCGCCTGGTCATCGTCAACCTGCAGCCCACCAAGCACGA
CCGCCATGCTGACCTCCGCATCCATGGCTACGTTGACGAGGTCATGACCC
GGCTCATGGAGCACCTGGGGCTGGAGATCCCCGCCTGGGACGGCCCCCGT
GTGCTGGAGAGGGCGCTGCCACCCCTGCCCCGCCCGCCCACCCCCAAGCT
GGAGCCCAAGGAGGAATCTCCCACCCGGATCAACGGCTCTATCCCCGCCG
GCCCCAAGCAGGAGCCCTGCGCCCAGCACAACGGCTCAGAGCCCGCCAGC
CCCAAACGGGAGCGGCCCACCAGCCCTGCCCCCCACAGACCCCCCAAAAG
GGTGAAGGCCAAGGCGGTCCCCAGCTGACCAGGGTGCTTGGGGAGGGTGG
GGCTTTTTGTAGAAACTGTGGATTCTTTTTCTCTCGTGGTCTCACTTTGT
TACTTGTTTCTGTCCCCGGGAGCCTCAGGGCTCTGAGAGCTGTGCTCCAG
GCCAGGGGTTACACCTGCCCTCCGTGGTCCCTCCCTGGGCTCCAGGGGCC
TCTGGTGCGGTTCCGGGAAGAAGCCACACCCCAGAGGTGACAGCTGAGCC
CCTGCCACACCCCAGCCTCTGACTTGCTGTGTTGTCCAGAGGTGAGGCTG
GGCCCTCCCTGGTCTCCAGCTTAAACAGGAGTGAACTCCCTCTGTCCCCA
GGGCCTCCCTTCTGGGCCCCCTACAGCCCACCCTACCCCTCCTCCATGGG
CCCTGCAGGAGGGGAGACCCACCTTGAAGTGGGGGATCAGTAGAGGCTTG
CACTGCCTTTGGGGCTGGAGGGAGACGTGGGTCCACCAGGCTTCTGGAAA
AGTCCTCAATGCAATAAAAACAATTTCTTTCTTGCAAA
hSIRT7

SEQ ID NO:111

>gi|7706711|ref|NM_016538.1|Homo sapiens sirtuin
silent mating type information regulation 2
homolog 7 (S. cerevisiae) (SIRT7), mRNA
GCGGAAGCGGAAGAGCAGGTCTCCAGGGGAGCGATGGCAGCCGGGGGTCT
GAGCCGCTCCGAGCGCAAAGCGGCGGAGCGGGTCCGGAGGTTGCGGGAGG
AGCAGCAGAGGGAGCGCCTCCGCCAGGTGTCGCGCATCCTGAGGAAGGCG
GCGGCGGAGCGCAGCGCCGAGGAGGGCCGGCTGCTGGCCGAGAGCGCGGA
CCTGGTAACGGAGCTGCAGGGCCGGAGCCGGCGGCGCGAGGGCCTGAAGC
GGCGGCAGGAGGAGGTGTGCGACGACCCGGAGGAGCTGCGGGGGAAGGTC
CGGGAGCTGGCCAGCGCCGTCCGGAACGCCAAATACTTGGTCGTCTACAC
AGGCGCGGGAATCAGCACGGCAGCGTCTATCCCAGACTACCGGGGCCCTA
ATGGAGTGTGGACACTGCTTCAGAAAGGGAGAAGCGTTAGTGCTGCCGAC
CTGAGCGAGGCCGAGCCAACCCTCACCCACATGAGCATCACCCGTCTGCA
TGAGCAGAAGCTGGTGCAGCATGTGGTGTCTCAGAACTGTGACGGGCTCC
ACCTGAGGAGTGGGCTGCCGCGCACGGCCATCTCCGAGCTCCACGGGAAC
ATGTACATTGAAGTCTGTACCTCCTGCGTTCCCAACAGGGAGTACGTGCG
GGTGTTCGATGTGACGGAGCGCACTGCCCTCCACAGACACCAGACAGGCC
GGACCTGCCACAAGTGTGGGACCCAGCTGCGGGACACCATTGTGCACTTT
GGGGAGAGGGGGACGTTGGGGCAGCCTCTGAACTGGGAAGCGGCGACCGA
GGCTGCCAGCAGAGCAGACACCATCCTGTGTCTAGGGTCCAGCCTGAAGG
TTCTAAAGAAGTACCCACGCCTCTGGTGCATGACCAAGCCCCCTAGCCGG
CGGCCGAAGCTTTACATCGTGAACCTGCAGTGGACCCCGAAGGATGACTG
GGCTGCCCTGAAGCTACATGGGAAGTGTGATGACGTCATGCGGCTCCTCA
TGGCCGAGCTGGGCTTGGAGATCCCCGCCTATAGCAGGTGGCAGGATCCC
ATTTTCTCACTGGCGACTCCCCTGCGTGCTGGTGAAGAAGGCAGCCACAG
TCGGAAGTCGCTGTGCAGAAGCAGAGAGGAGGCCCCGCCTGGGGACCGGG
GTGCACCGCTTAGCTCGGCCCCCATCCTAGGGGGCTGGTTTGGCAGGGGC
TGCACAAAACGCACAAAAAGGAAGAAAGTGACGTAATCACGTGCTCGATG
AAGAACAGTTGGCACTTTGCAGATGGCCAGTGTCACGGTGAAGGCTGGGT
TGCCCCCACGGGTCTAGGGAGAACGAACTCTTTGGGGATGACATTTTCAC
CGTGACATTTTTAGCCATTTGTCCTTGAGGAAGCCCCTTGCACTGCTGCG
GTTGTACCCTGATACGGCCTGGCCATCGAGGACACCTGCCCATCCGGCCT
CTGTGTCAAGAGGTGGCAGCCGCACCTTTCTGTGAGAACGGAACTCGGGT
TATTTCAGCCCCGGCCTGCAGAGTGGAAGCGCCCAGCGGCCTTTCCTCGC
TCACCAGGCCAGTCTCAGGGCCTCACCGTATTTCTACTACTACTTAATGA
AAAAGTGTGAACTTTATAGAATCCTCTCTGTACTGGATGTGCGGCAGAGG
GGTGGCTCCGAGCCTCGGCTCTATGCAGACCTTTTTATTTCTATTAAACG
TTTCTGCACTGGCAAAAA
MECP2

SEQ ID NO:112

>gi|7710148|ref|NM_004992.2|Homo sapiens methyl
CpG binding protein 2 (Rett syndrome) (MECP2),
mRNA
CCGGAAAATGGCCGCCGCCGCCGCCGCCGCGCCGAGCGGAGGAGGAGGAG
GAGGCGAGGAGGAGAGACTGCTCCATAAAAATACAGACTCACCAGTTCCT
GCTTTGATGTGACATGTGACTCCCCAGAATACACCTTGCTTCTGTAGACC
AGCTCCAACAGGATTCCATGGTAGCTGGGATGTTAGGGCTCAGGGAAGAA
AAGTCAGAAGACCAGGACCTCCAGGGCCTCAAGGACAAACCCCTCAAGTT
TAAAAAGGTGAAGAAAGATAAGAAAGAAGAGAAAGAGGGCAAGCATGAGC
CCGTGCAGCCATCAGCCCACCACTCTGCTGAGCCCGCAGAGGCAGGCAAA
GCAGAGACATCAGAAGGGTCAGGCTCCGCCCCGGCTGTGCCGGAAGCTTC
TGCCTCCCCCAAACAGCGGCGCTCCATCATCCGTGACCGGGGACCCATGT
ATGATGACCCCACCCTGCCTGAAGGCTGGACACGGAAGCTTAAGCAAAGG
AAATCTGGCCGCTCTGCTGGGAAGTATGATGTGTATTTGATCAATCCCCA
GGGAAAAGCCTTTCGCTCTAAAGTGGAGTTGATTGCGTACTTCGAAAAGG
TAGGCGACACATCCCTGGACCCTAATGATTTTGACTTCACGGTAACTGGG
AGAGGGAGCCCCTCCCGGCGAGAGCAGAAACCACCTAAGAAGCCCAAATC
TCCCAAAGCTCCAGGAACTGGCAGAGGCCGGGGACGCCCCAAAGGGAGCG
GCACCACGAGACCCAAGGCGGCCACGTCAGAGGGTGTGCAGGTGAAAAGG
GTCCTGGAGAAAAGTCCTGGGAAGCTCCTTGTCAAGATGCCTTTTCAAAC
TTCGCCAGGGGGCAAGGCTGAGGGGGGTGGGGCCACCACATCCACCCAGG
TCATGGTGATCAAACGCCCCGGCAGGAAGCGAAAAGCTGAGGCCGACCCT
CAGGCCATTCCCAAGAAACGGGGCCGAAAGCCGGGGAGTGTGGTGGCAGC
CGCTGCCGCCGAGGCCAAAAAGAAAGCCGTGAAGGAGTCTTCTATCCGAT
CTGTGCAGGAGACCGTACTCCCCATCAAGAAGCGCAAGACCCGGGAGACG
GTCAGCATCGAGGTCAAGGAAGTGGTGAAGCCCCTGCTGGTGTCCACCCT
CGGTGAGAAGAGCGGGAAAGGACTGAAGACCTGTAAGAGCCCTGGGCGGA
AAAGCAAGGAGAGCAGCCCCAAGGGGCGCAGCAGCAGCGCCTCCTCACCC
CCCAAGAAGGAGCACCACCACCATCACCACCACTCAGAGTCCCCAAAGGC
CCCCGTGCCACTGCTCCCACCCCTGCCCCCACCTCCACCTGAGCCCGAGA
GCTCCGAGGACCCCACCAGCCCCCCTGAGCCCCAGGACTTGAGCAGCAGC
GTCTGCAAAGAGGAGAAGATGCCCAGAGGAGGCTCACTGGAGAGCGACGG
CTGCCCCAAGGAGCCAGCTAAGACTCAGCCCGCGGTTGCCACCGCCGCCA
CGGCCGCAGAAAAGTACAAACACCGAGGGGAGGGAGAGCGCAAAGACATT
GTTTCATCCTCCATGCCAAGGCCAAACAGAGAGGAGCCTGTGGACAGCCG
GACGCCCGTGACCGAGAGAGTTAGCTGACTTTACACGGAGCGGATTGCAA
AGCAAACCAACAAGAATAAAGGCAGCTGTTGTCTCTTCTCCTTATGGGTA
GGGCTCTGACAAAGCTTCCCGATTAACTGAAATAAAAAATATTTTTTTTT
CTTTCAGTAAACTTAGAGTTTCGTGGCTTCAGGGTGGGAGTAGTTGGAGC
ATTGGGGATGTTTTTCTTACCGACAAGCACAGTCAGGTTGAAGACCTAAC
CAGGGCCAGAAGTAGCTTTGCACTTTTCTAAACTAGGCTCCTTCAACAAG
GCTTGCTGCAGATACTACTGACCAGACAAGCTGTTGACCAGGCACCTCCC
CTCCCGCCCAAACCTTTCCCCCATGTGGTCGTTAGAGACAGAGCGACAGA
GCAGTTGAGAGGACACTCCCGTTTTCGGTGCCATCAGTGCCCCGTCTACA
GCTCCCCCAGCTCCCCCCACCTCCCCCACTCCCAACCACGTTGGGACAGG
GAGGTGTGAGGCAGGAGAGACAGTTGGATTCTTTAGAGAAGATGGATATG
ACCAGTGGCTATGGCCTGTGCGATCCCACCCGTGGTGGCTCAAGTCTGGC
CCCACACCAGCCCCAATCCAAAACTGGCAAGGACGCTTCACAGGACAGGA
AAGTGGCACCTGTCTGCTCCAGCTCTGGCATGGCTAGGAGGGGGGAGTCC
CTTGAACTACTGGGTGTAGACTGGCCTGAACCACAGGAGAGGATGGCCCA
GGGTGAGGTGGCATGGTCCATTCTCAAGGGACGTCCTCCAACGGGTGGCG
CTAGAGGCCATGGAGGCAGTAGGACAAGGTGCAGGCAGGCTGGCCTGGGG
TCAGGCCGGGCAGAGCACAGCGGGGTGAGAGGGATTCCTAATCACTCAGA
GCAGTCTGTGACTTAGTGGACAGGGGAGGGGGCAAAGGGGGAGGAGAAGA
AAATGTTCTTCCAGTTACTTTCCAATTCTCCTTTAGGGACAGCTTAGAAT
TATTTGCACTATTGAGTCTTCATGTTCCCACTTCAAAACAAACAGATGCT
CTGAGAGCAAACTGGCTTGAATTGGTGACATTTAGTCCCTCAAGCCACCA
GATGTGACAGTGTTGAGAACTACCTGGATTTGTATATATACCTGCGCTTG
TTTTAAAGTGGGCTCAGCACATAGGGTTCCCACGAAGCTCCGAAACTCTA
AGTGTTTGCTGCAATTTTATAAGGACTTCCTGATTGGTTTCTCTTCTCCC
CTTCCATTTCTGCCTTTTGTTCATTTCATCCTTTCACTTCTTTCCCTTCC
TCCGTCCTCCTCCTTCCTAGTTCATCCCTTCTCTTCCAGGCAGCCGCGGT
GCCCAACCACACTTGTCGGCTCCAGTCCCCAGAACTCTGCCTGCCCTTTG
TCCTCCTGCTGCCAGTACCAGCCCCACCCTGTTTTGAGCCCTGAGGAGGC
CTTGGGCTCTGCTGAGTCCGACCTGGCCTGTCTGTGAAGAGCAAGAGAGC
AGCAAGGTCTTGCTCTCCTAGGTAGCCCCCTCTTCCCTGGTAAGAAAAAG
CAAAAGGCATTTCCCACCCTGAACAACGAGCCTTTTCACCCTTCTACTCT
AGAGAAGTGGACTGGAGGAGCTGGGCCCGATTTGGTAGTTGAGGAAAGCA
CAGAGGCCTCCTGTGGCCTGCCAGTCATCGAGTGGCCCAACAGGGGCTCC
ATGCCAGCCGACCTTGACCTCACTCAGAAGTCCAGAGTCTAGCGTAGTGC
AGCAGGGCAGTAGCGGTACCAATGCAGAACTCCCAAGACCCGAGCTGGGA
CCAGTACCTGGGTCCCCAGCCCTTCCTCTGCTCCCCCTTTTCCCTCGGAG
TTCTTCTTGAATGGCAATGTTTTGCTTTTGCTCGATGCAGACAGGGGGCC
AGAACACCACACATTTCACTGTCTGTCTGGTCCATAGCTGTGGTGTAGGG
GCTTAGAGGCATGGGCTTGCTGTGGGTTTTTAATTGATCAGTTTTCATGT
GGGATCCCATCTTTTTAACCTCTGTTCAGGAAGTCCTTATCTAGCTGCAT
ATCTTCATCATATTGGTATATCCTTTTCTGTGTTTACAGAGATGTCTCTT
ATATCTAAATCTGTCCAACTGAGAAGTACCTTATCAAAGTAGCAAATGAG
ACAGCAGTCTTATGCTTCCAGAAACACCCACAGGCATGTCCCATGTGAGC
TGCTGCCATGAACTGTCAAGTGTGTGTTGTCTTGTGTATTTCAGTTATTG
TCCCTGGCTTCCTTACTATGGTGTAATCATGAAGGAGTGAAACATCATAG
AAACTGTCTAGCACTTCCTTGCCAGTCTTTAGTGATCAGGAACCATAGTT
GACAGTTCCAATCAGTAGCTTAAGAAAAAACCGTGTTTGTCTCTTCTGGA
ATGGTTAGAAGTGAGGGAGTTTGCCCCGTTCTGTTTGTAGAGTCTCATAG
TTGGACTTTCTAGCATATATGTGTCCATTTCCTTATGCTGTAAAAGCAAG
TCCTGCAACCAAACTCCCATCAGCCCAATCCCTGATCCCTGATCCCTTCC
ACCTGCTCTGCTGATGACCCCCCCAGCTTCACTTCTGACTCTTCCCCAGG
AAGGGAAGGGGGGTCAGAAGAGAGGGTGAGTCCTCCAGAACTCTTCCTCC
AAGGACAGAAGGCTCCTGCCCCCATAGTGGCCTCGAACTCCTGGCACTAC
CAAAGGACACTTATCCACGAGAGCGCAGCATCCGACCAGGTTGTCACTGA
GAAGATGTTTATTTTGGTCAGTTGGGTTTTTATGTATTATACTTAGTCAA
ATGTAATGTGGCTTCTGGAATCATTGTCCAGAGCTGCTTCCCCGTCACCT
GGGCGTCATCTGGTCCTGGTAAGAGGAGTGCGTGGCCCACCAGGCCCCCC
TGTCACCCATGACAGTTCATTCAGGGCCGATGGGGCAGTCGTGGTTGGGA
ACACAGCATTTCAAGCGTCACTTTATTTCATTCGGGCCCCACCTGCAGCT
CCCTCAAAGAGGCAGTTGCCCAGCCTCTTTCCCTTCCAGTTTATTCCAGA
GCTGCCAGTGGGGCCTGAGGCTCCTTAGGGTTTTCTCTCTATTTCCCCCT
TTCTTCCTCATTCCCTCGTCTTTCCCAAAGGCATCACGAGTCAGTCGCCT
TTCAGCAGGCAGCCTTGGCGGTTTATCGCCCTGGCAGGCAGGGGCCCTGC
AGCTCTCATGCTGCCCCTGCCTTGGGGTCAGGTTGACAGGAGGTTGGAGG
GAAAGCCTTAAGCTGCAGGATTCTCACCAGCTGTGTCCGGCCCAGTTTTG
GGGTGTGACCTCAATTTCAATTTTGTCTGTACTTGAACATTATGAAGATG
GGGGCCTCTTTCAGTGAATTTGTGAACAGCAGAATTGACCGACAGCTTTC
CAGTACCCATGGGGCTAGGTCATTAAGGCCACATCCACAGTCTCCCCCAC
CCTTGTTCCAGTTGTTAGTTACTACCTCCTCTCCTGACAATACTGTATGT
CGTCGAGCTCCCCCCAGGTCTACCCCTCCCGGCCCTGCCTGCTGGTGGGC
TTGTCATAGCCAGTGGGATTGCCGGTCTTGACAGCTCAGTGAGCTGGAGA
TACTTGGTCACAGCCAGGCGCTAGCACAGCTCCCTTCTGTTGATGCTGTA
TTCCCATATCAAAAGACACAGGGGACACCCAGAAACGCCACATCCCCCAA
TCCATCAGTGCCAAACTAGCCAACGGCCCCAGCTTCTCAGCTCGCTGGAT
GGCGGAAGCTGCTACTCGTGAGCGCCAGTGCGGGTGCAGACAATCTTCTG
TTGGGTGGCATCATTCCAGGCCCGAAGCATGAACAGTGCACCTGGGACAG
GGAGCAGCCCCAAATTGTCACCTGCTTCTCTGCCCAGCTTTTCATTGCTG
TGACAGTGATGGCGAAAGAGGGTAATAACCAGACACAAACTGCCAAGTTG
GGTGGAGAAAGGAGTTTCTTTAGCTGACAGAATCTCTGAATTTTAAATCA
CTTAGTAAGCGGCTCAAGCCCAGGAGGGAGCAGAGGGATACGAGCGGAGT
CCCCTGCGCGGGACCATCTGGAATTGGTTTAGCCCAAGTGGAGCCTGACA
GCCAGAACTCTGTGTCCCCCGTCTAACCACAGCTCCTTTTCCAGAGCATT
CCAGTCAGGCTCTCTGGGCTGACTGGGCCAGGGGAGGTTACAGGTACCAG
TTCTTTAAGAAGATCTTTGGGCATATACATTTTTAGCCTGTGTCATTGCC
CCAAATGGATTCCTGTTTCAAGTTCACACCTGCAGATTCTAGGACCTGTG
TCCTAGACTTCAGGGAGTCAGCTGTTTCTAGAGTTCCTACCATGGAGTGG
GTCTGGAGGACCTGCCCGGTGGGGGGGCAGAGCCCTGCTCCCTCCGGGTC
TTCCTACTCTTCTCTCTGCTCTGACGGGATTTGTTGATTCTCTCCATTTT
GGTGTCTTTCTCTTTTAGATATTGTATCAATCTTTAGAAAAGGCATAGTC
TACTTGTTATAAATCGTTAGGATACTGCCTCCCCCAGGGTCTAAAATTAC
ATATTAGAGGGGAAAAGCTGAACACTGAAGTCAGTTCTCAACAATTTAGA
AGGAAAACCTAGAAAACATTTGGCAGAAAATTACATTTCGATGTTTTTGA
ATGAATACGAGCAAGCTTTTACAACAGTGCTGATCTAAAAATACTTAGCA
CTTGGCCTGAGATGCCTGGTGAGCATTACAGGCAAGGGGAATCTGGAGGT
AGCCGACCTGAGGACATGGCTTCTGAACCTGTCTTTTGGGAGTGGTATGG
AAGGTGGAGCGTTCACCAGTGACCTGGAAGGCCCAGCACCACCCTCCTTC
CCACTCTTCTCATCTTGACAGAGCCTGCCCCAGCGCTGACGTGTCAGGAA
AACACCCAGGGAACTAGGAAGGCACTTCTGCCTGAGGGGCAGCCTGCCTT
GCCCACTCCTGCTCTGCTCGCCTCGGATCAGCTGAGCCTTCTGAGCTGGC
CTGTCACTGCCTCCCCAAGGCCCCCTGCCTGCCCTGTCAGGAGGCAGAAG
GAAGCAGGTGTGAGGGCAGTGCAAGGAGGGAGCACAACCCCCAGCTCCCG
CTCCGGGCTCCGACTTGTGCACAGGCAGAGCCCAGACCCTGGAGGAAATC
CTACCTTTGAATTCAAGAACATTTGGGGAATTTGGAAATCTCTTTGCCCC
CAAACCCCCATTCTGTCCTACCTTTAATCAGGTCCTGCTCAGCAGTGAGA
GCAGATGAGGTGAAAAGGCCAAGAGGTTTGGCTCCTGCCCACTGATAGCC
CCTCTCCCCGCAGTGTTTGTGTGTCAAGTGGCAAAGCTGTTCTTCCTGGT
GACCCTGATTATATCCAGTAACACATAGACTGTGCGCATAGGCCTGCTTT
GTCTCCTCTATCCTGGGCTTTTGTTTTGCTTTTTAGTTTTGCTTTTAGTT
TTTCTGTCCCTTTTATTTAACGCACCGACTAGACACACAAAGCAGTTGAA
TTTTTATATATATATCTGTATATTGCACAATTATAAACTCATTTTGCTTG
TGGCTCCACACACACAAAAAAAGACCTGTTAAAATTATACCTGTTGCTTA
ATTACAATATTTCTGATAACCATAGCATAGGACAAGGGAAAATAAAAAAA
GAAAAAAAAGAAAAAAAAACGACAAATCTGTCTGCTGGTCACTTCTTCTG
TCCAAGCAGATTCGTGGTCTTTTCCTCGCTTCTTTCAAGGGCTTTCCTGT
GCCAGGTGAAGGAGGCTCCAGGCAGCACCCAGGTTTTGCACTCTTGTTTC
TCCCGTGCTTGTGAAAGAGGTCCCAAGGTTCTGGGTGCAGGAGCGCTCCC
TTGACCTGCTGAAGTCCGGAACGTAGTCGGCACAGCCTGGTCGCCTTCCA
CCTCTGGGAGCTGGAGTCCACTGGGGTGGCCTGACTCCCCCAGTCCCCTT
CCCGTGACCTGGTCAGGGTGAGCCCATGTGGAGTCAGCCTCGCAGGCCTC
CCTGCCAGTAGGGTCCGAGTGTGTTTCATCCTTCCCACTCTGTCGAGCCT
GGGGGCTGGAGCGGAGACGGGAGGCCTGGCCTGTCTCGGAACCTGTGAGC
TGCACCAGGTAGAACGCCAGGGACCCCAGAATCATGTGCGTCAGTCCAAG
GGGTCCCCTCCAGGAGTAGTGAAGACTCCAGAAATGTCCCTTTCTTCTCC
CCCATCCTACGAGTAATTGCATTTGCTTTTGTAATTCTTAATGAGCAATA
TCTGCTAGAGAGTTTAGCTGTAACAGTTCTTTTTGATCATCTTTTTTTAA
TAATTAGAAACACCAAAAAAATCCAGAAACTTGTTCTTCCAAAGCAGAGA
GCATTATAATCACCAGGGCCAAAAGCTTCCCTCCCTGCTGTCATTGCTTC
TTCTGAGGCCTGAATCCAAAAGAAAAACAGCCATAGGCCCTTTCAGTGGC
CGGGCTACCCGTGAGCCCTTCGGAGGACCAGGGCTGGGGCAGCCTCTGGG
CCCACATCCGGGGCCAGCTCCGGCGTGTGTTCAGTGTTAGCAGTGGGTCA
TGATGCTCTTTCCCACCCAGCCTGGGATAGGGGCAGAGGAGGCGAGGAGG
CCGTTGCCGCTGATGTTTGGCCGTGAACAGGTGGGTGTCTGCGTGCGTCC
ACGTGCGTGTTTTCTGACTGACATGAAATCGACGCCCGAGTTAGCCTCAC
CCGGTGACCTCTAGCCCTGCCCGGATGGAGCGGGGCCCACCCGGTTCAGT
GTTTCTGGGGAGCTGGACAGTGGAGTGCAAAAGGCTTGCAGAACTTGAAG
CCTGCTCCTTCCCTTGCTACCACGGCCTCCTTTCCGTTTGATTTGTCACT
GCTTCAATCAATAACAGCCGCTCCAGAGTCAGTAGTCAATGAATATATGA
CCAAATATCACCAGGACTGTTACTCAATGTGTGCCGAGCCCTTGCCCATG
CTGGGCTCCCGTGTATCTGGACACTGTAACGTGTGCTGTGTTTGCTCCCC
TTCCCCTTCCTTCTTTGCCCTTTACTTGTCTTTCTGGGGTTTTTCTGTTT
GGGTTTGGTTTGGTTTTTATTTCTCCTTTTGTGTTCCAAACATGAGGTTC
TCTCTACTGGTCCTCTTAACTGTGGTGTTGAGGCTTATATTTGTGTAATT
TTTGGTGGGTGAAAGGAATTTTGCTAAGTAAATCTCTTCTGTGTTTGAAC
TGAAGTCTGTATTGTAACTATGTTTAAAGTAATTGTTCCAGAGACAAATA
TTTCTAGACACTTTTTCTTTACAAACAAAAGCATTCGGAGGGAGGGGGAT
GGTGACTGAGATGAGAGGGGAGAGCTGAACAGATGACCCCTGCCCAGATC
AGCCAGAAGCCACCCAAAGCAGTGGAGCCCAGGAGTCCCACTCCAAGCCA
GCAAGCCGAATAGCTGATGTGTTGCCACTTTCCAAGTCACTGCAAAACCA
GGTTTTGTTCCGCCCAGTGGATTCTTGTTTTGCTTCCCCTCCCCCCGAGA
TTATTACCACCATCCCGTGCTTTTAAGGAAAGGCAAGATTGATGTTTCCT
TGAGGGGAGCCAGGAGGGGATGTGTGTGTGCAGAGCTGAAGAGCTGGGGA
GAATGGGGCTGGGCCCACCCAAGCAGGAGGCTGGGACGCTCTGCTGTGGG
CACAGGTCAGGCTAATGTTGGCAGATGCAGCTCTTCCTGGACAGGCCAGG
TGGTGGGCATTCTCTCTCCAAGGTGTGCCCCGTGGGCATTACTGTTTAAG
ACACTTCCGTCACATCCCACCCCATCCTCCAGGGCTCAACACTGTGACAT
CTCTATTCCCCACCCTCCCCTTCCCAGGGCAATAAAATGACCATGGAGGG
GGCTTGCACTCTCTTGGCTGTCACCCGATCGCCAGCAAAACTTAGATGTG
AGAAAACCCCTTCCCATTCCATGGCGAAAACATCTCCTTAGAAAAGCCAT
TACCCTCATTAGGCATGGTTTTGGGCTCCCAAAACACCTGACAGCCCCTC
CCTCCTCTGAGAGGCGGAGAGTGCTGACTGTAGTGACCATTGCATGCCGG
GTGCAGCATCTGGAAGAGCTAGGCAGGGTGTCTGCCCCCTCCTGAGTTGA
AGTCATGCTCCCCTGTGCCAGCCCAGAGGCCGAGAGCTATGGACAGCATT
GCCAGTAACACAGGCCACCCTGTGCAGAAGGGAGCTGGCTCCAGCCTGGA
AACCTGTCTGAGGTTGGGAGAGGTGCACTTGGGGCACAGGGAGAGGCCGG
GACACACTTAGCTGGAGATGTCTCTAAAAGCCCTGTATCGTATTCACCTT
CAGTTTTTGTGTTTTGGGACAATTACTTTAGAAAATAAGTAGGTCGTTTT
AAAAACAAAAATTATTGATTGCTTTTTTGTAGTGTTCAGAAAAAAGGTTC
TTTGTGTATAGCCAAATGACTGAAAGCACTGATATATTTAAAAACAAAAG
GCAATTTATTAAGGAAATTTGTACCATTTCAGTAAACCTGTCTGAATGTA
CCTGTATACGTTTCAAAAACACCCCCCCCCCACTGAATCCCTGTAACCTA
TTTATTATATAAAGAGTTTGCCTTATAAATTT
ZNF145

SEQ ID NO:113

>gi|5174752|ref|NM_006006.1|Homo sapiens zinc
finger protein 145 (Kruppel-like, expressed in
promyelocytic leukemia) (ZNF145), mRNA
CAGGAAGCCCACCCAGCCCCGCCACGCAGAGCCCAGAAGGAAAGAAAGCC
TCATGCCTGAGCCGAGGGGAGCACCATGGATCTGACAAAAATGGGCATGA
TCCAGCTGCAGAACCCTAGCCACCCCACGGGGCTACTGTGCAAGGCCAAC
CAGATGCGGCTGGCCGGGACTTTGTGCGATGTGGTCATCATGGTGGACAG
CCAGGAGTTCCACGCCCACCGGACGGTGCTGGCCTGCACCAGCAAGATGT
TTGAGATCCTCTTCCACCGCAATAGTCAACACTATACTTTGGACTTCCTC
TCGCCAAAGACCTTCCAGCAGATTCTGGAGTATGCATATACAGCCACGCT
GCAAGCCAAGGCGGAGGACCTGGATGACCTGCTGTATGCGGCCGAGATCC
TGGAGATCGAGTACCTGGAGGAACAGTGCCTGAAGATGCTGGAGACCATC
CAGGCCTCAGACGACAATGACACGGAGGCCACCATGGCCGATGGCGGGGC
CGAGGAAGAAGAGGACCGCAAGGCTCGGTACCTCAAGAACATCTTCATCT
CGAAGCATTCCAGCGAGGAGAGTGGGTATGCCAGTGTGGCTGGACAGAGC
CTCCCTGGGCCCATGGTGGACCAGAGCCCTTCAGTCTCCACTTCATTTGG
TCTTTCAGCCATGAGTCCCACCAAGGCTGCAGTGGACAGTTTGATGACCA
TAGGACAGTCTCTCCTGCAGGGAACTCTTCAGCCACCTGCAGGGCCCGAG
GAGCCAACTCTGGCTGGGGGTGGGCGGCACCCTGGGGTGGCTGAGGTGAA
GACGGAGATGATGCAGGTGGATGAGGTGCCCAGCCAGGACAGCCCTGGGG
CAGCCGAGTCCAGCATCTCAGGAGGGATGGGGGACAAGGTTGAGGAAAGA
GGCAAAGAGGGGCCTGGGACCCCGACTCGAAGCAGCGTCATCACCAGTGC
TAGGGAGCTACACTATGGGCGAGAGGAGAGTGCCGAGCAGGTGCCACCCC
CAGCTGAGGCTGGCCAGGCCCCCACTGGCCGACCTGAGCACCCAGCACCC
CCGCCTGAGAAGCATCTGGGCATCTACTCCGTGTTGCCCAACCACAAGGC
TGACGCTGTATTGAGCATGCCGTCTTCCGTGACCTCTGGCCTCCACGTGC
AGCCTGCCCTGGCTGTCTCCATGGACTTCAGCACCTATGGGGGGCTGCTG
CCCCAGGGCTTCATCCAGAGGGAGCTGTTCAGCAAGCTGGGGGAGCTGGC
TGTGGGCATGAAGTCAGAGAGCCGGACCATCGGAGAGCAGTGCAGCGTGT
GTGGGGTCGAGCTTCCTGATAACGAGGCTGTGGAGCAGCACAGGAAGCTG
CACAGTGGGATGAAGACGTACGGGTGCGAGCTCTGCGGGAAGCGGTTCCT
GGATAGTTTGCGGCTGAGAATGCACTTACTGGCTCATTCAGCGGGTGCCA
AAGCCTTTGTCTGTGATCAGTGCGGTGCACAGTTTTCGAAGGAGGATGCC
CTGGAGACACACAGGCAGACCCATACTGGCACTGACATGGCCGTCTTCTG
TCTGCTGTGTGGGAAGCGCTTCCAGGCGCAGAGCGCACTGCAGCAGCACA
TGGAGGTCCACGCGGGCGTGCGCAGCTACATCTGCAGTGAGTGCAACCGC
ACCTTCCCCAGCCACACGGCTCTCAAACGCCACCTGCGCTCACATACAGG
CGACCACCCCTACGAGTGTGAGTTCTGTGGCAGCTGCTTCCGGGATGAGA
GCACACTCAAGAGCCACAAACGCATCCACACGGGTGAGAAACCCTACGAG
TGCAATGGCTGTGACAAGAAGTTCAGCCTCAAGCATCAGCTGGAGACGCA
CTATAGGGTGCACACAGGTGAGAAGCCCTTTGAGTGTAAGCTCTGCCACC
AGCGCTCCCGGGACTACTCGGCCATGATCAAGCACCTGAGAACGCACAAC
GGCGCCTCGCCCTACCAGTGCACCATCTGCACAGAGTACTGCCCCAGCCT
CTCCTCCATGCAGAAGCACATGAAGGGCCACAAGCCCGAGGAGATCCCGC
CCGACTGGAGGATAGAGAAGACGTACCTCTACCTGTGCTATGTGTGAAGG
GAGGCCCGCGGCGGTGGAGCCGAGCGGGGAGCCAGGAAAGAAGAGTTGGA
GTGAGATGAAGGAAGGACTA
TFDP1

SEQ ID NO:114

>gi|6005899|ref|NM_007111.1|Homo sapiens tran-
scription factor Dp-1 (TFDP1), mRNA
GGAATTCCGTAGCTATTGATTTCCCGGATCTGGTAACATGGCAAAAGATG
CCGGTCTAATTGAAGCCAACGGAGAACTCAAGGTCTTCATAGACCAGAAC
CTTAGTCCCGGGAAAGGCGTGGTGTCCCTCGTGGCCGTTCACCCCTCCAC
CGTCAACCCGCTCGGGAAGCAGCTCTTGCCAAAAACCTTTGGACAGTCCA
ATGTCAACATTGCCCAGCAAGTGGTAATTGGTACGCCTCAGAGACCGGCA
GCGTCAAACACCCTGGTGGTAGGAAGCCCACACACCCCCAGCACTCACTT
TGCCTCTCAGAACCAGCCTTCCGACTCCTCACCTTGGTCTGCCGGGAAGC
GCAACAGGAAAGGAGAGAAGAATGGCAAGGGCCTACGGCATTTCTCCATG
AAGGTCTGCGAGAAGGTGCAGAGGAAAGGGACCACTTCCTACAACGAAGT
GGCAGACGAGCTGGTTGCGGAGTTCAGTGCTGCCGACAACCACATCTTAC
CAAACGAGTCAGCTTATGACCAGAAAAACATAAGACGGCGCGTCTACGAT
GCCTTAAACGTGCTAATGGCCATGAACATCATCTCCAAGGAGAAGAAGGA
GATCAAGTGGATTGGTCTGCCCACCAACTCGGCTCAGGAATGTCAGAACT
TAGAGGTGGAAAGACAGAGGAGACTTGAAAGAATAAAACAGAAACAGTCT
CAACTTCAAGAACTTATTCTACAGCAAATTGCCTTCAAGAACCTGGTGCA
GAGAAACCGGCATGCGGAGCAGCAGGCCAGCCGGCCACCGCCACCCAACT
CAGTCATCCACCTGCCCTTCATCATCGTCAACACCAGCAAGAAGACGGTC
ATCGACTGCAGCATCTCCAATGACAAATTTGAGTATCTGTTTAATTTTGA
CAACACATTTGAAATCCACGATGACATAGAAGTGCTGAAGCGGATGGGCA
TGGCTTGCGGGCTGGAGTCGGGGAGCTGCTCTGCCGAAGACCTTAAAATG
GCCAGAAGTCTGGTCCCCAAGGCTCTGGAGCCATACGTGACAGAAATGGC
TCAGGGAACTGTTGGAGGCGTGTTCATCACGACGGCAGGTTCCACGTCTA
ACGGCACAAGGTTCTCTGCCAGTGACCTGACCAACGGTGCAGATGGGATG
CTGGCCACAAGCTCCAATGGGTCTCAGTACAGCGGCTCCAGGGTGGAGAC
TCCGGTGTCCTACGTCGGGGAGGACGACGAGGAGGACGATGACTTCAACG
AGAATGACGAGGACGACTGACGTCCTCCCCACTTCAGATTCGGCTTCAGG
AAAACGTTTAGCGAAAAGAAACTTTTTTTTTAATGTGGGTTTTCTGTTTC
CTTTTGGCCTAGTCCCAAGAAGATATTGGTAAGCTATTGAATTTAGATAT
GCACCTCTGATAAGCAAGGATTGTTTCCCGTAGATTAGGA
SAP30

SEQ ID NO:115

>gi|4506782|ref|NM_003864.1|Homo sapiens sin3-
associated polypeptide, 30kD (SAP30), mRNA
CCCCCATGTGACAGTGACGGGGTCCCCGCTCCAGGAGACGCTCGAGTCTG
CGTCCCGGCCCTCAGCACTGTCCACTGTTTCGGTGCCAGCAGAGACCAGC
AGGCCCGGGACAGTTGGTGTTTGGCCGTGCCGCTGTCTAACTTGGTGTGC
AGAGTGAATTGCCGCTGCCGGAGCGGAGAGAGGCGGAGCGGCCAGGAGAG
AGGGGATTTCTGTCAGCGCCGGCCTCGGGAGCTCGGAGACATGAACGGCT
TCACGCCTGACGAGATGAGCCGCGGCGGGGATGCGGCCGCCGCAGTGGCC
GCAGTGGTCGCTGCCGCGGCCGCCGCCGCCTCGGCGGGGAACGGGACCGG
CGCGGGCACCGGGGCTGAGGTGCCGGGCGCGGGGGCGGTCTCAGCGGCTG
GGCCCCCGGGGGCGGCCGGGCCGGGCCCCGGGCAACTGTGCTGCCTGCGG
GAGGATGGTGAGCGGTGCGGCCGGGCGGCAGGCAACGCCAGCTTCAGCAA
GAGGATCCAGAAGAGCATCTCCCAGAAGAAGGTGAAGATCGAGCTGGATA
AGAGCGCAAGGCATCTTTACATATGTGATTATCATAAAAACTTAATTCAG
AGTGTTCGAAACAGAAGAAAGAGAAAAGGGAGTGATGATGATGGAGGTGA
TTCACCTGTTCAAGATATTGATACCCCAGAGGTTGATTTATACCAATTAC
AAGTAAATACACTTAGGAGATACAAAAGACACTTCAAGCTACCAACCAGA
CCAGGACTTAATAAAGCACAACTTGTTGAGATAGTTGGTTGCCACTTTAG
GTCTATTCCAGTGAATGAAAAAGACACCTTAACATATTTCATCTACTCAG
TGAAGAATGACAAGAACAAATCAGATCTCAAGGTTGATAGTGGTGTTCAC
TAGGAGACGTGGAATTGAGACTAATAACTTGGATGTTAACACTGTTTACT
GTTTTTTCACATGTAGAAATGTTCTTTGTGTATTTTTTCTACAGAGGATT
TTCTCTGATTTTATTTTCTTTGTTTCTGACTCTAATAATTAGTTGGAAAC
TCATATAAAATGAGCTTTCCTAAATTAAATCTATTTTAAATAAAGGTTAT
TACTATTAAAAAAAAAAAAAAAAAA
SAP18

SEQ ID NO:116

>gi|12056471|ref|NM_005870.2|Homo sapiens sin3-
associated polypeptide, 18kD (SAP18), mRNA
AGGTTTGAACTGACTTTATTATTTTGTAAATGTGAATTTTACAAAGCGCT
TTACAATTAATGATCACATCCTTTTGTTTGTCATGGATTTCCACTGTCTG
AAACGGCTCTGAGCACGCTTGAAGCCCTCGGTTTCCCTGTTCGCTTTTGA
ATGTTTCAGTTTTAGTTATTGATACAATGTCAGCCATGGCTAAAAAGTAA
CAGTCTTGACTCTACCGAGTAACAGCACAAAAACAGAGTGAGGGCTCAGG
AAAACAAAACAAAGGCTTCCTCCTTAAAAAAAAGACAAAAAAAAAAAGCT
AAGCATCTGTGGCTGAAATCTAACTCAGTGGTACTGTGAAACCTTCCTTT
ACAGCACAGGAAAATTTATTTTTTAACAGTCGTGAGTTACAGTACTTTAA
CCCCTAAACAGACTCTTTAAAACAACCGTCTCCTTTTTAAAAGTCTCTTT
TTTCCAAACATTCCATCCGAAGGATGGATGCTCTACTTGCACCCAGTGCC
ATCCAAATGTTCAAGTCAAAAATATTTATACATTTTATACTTAGGCGAGC
GTCTCGCAGGCCGTAGGAGGAAGATGGCGGTGGAGTCGCGCGTTACCCAG
GAGGAAATTAAGAAGGAGCCAGAGAAACCGATCGACCGCGAGAAGACATG
CCCACTGTTGCTACGGGTCTTCACCACCAATAACGGCCGCCACCACCGAA
TGGACGAGTTCTCCCGGGGAAATGTACCGTCCAGCGAGTTGCAGATCTAC
ACTTGGATGGATGCAACTTTGAAAGAACTGACAAGCTTAGTAAAAGAAGT
CTACCCAGAAGCTAGAAAGAAGGGCACTCACTTCAATTTTGCAATCGTTT
TTACAGATGTTAAAAGACCTGGCTATCGAGTTAAGGAGATTGGCAGCACC
ATGTCTGGCAGAAAGGGGACTGATGATTCCATGACCCTGCAGTCGCAGAA
GTTCCAGATAGGAGATTACTTGGACATAGCAATTACCCCTCCAAATCGGG
CACCACCTCCTTCAGGGCGCATGAGACCATATTAAATTCTATTTACTATT
TGTTGAATTTATTTTTCCGTCAGTTATGTAAAATAAACATACTCTTCTTC
CTCCCCTGATTATTGCCATTAAGCCTTTAAATTCTAAACAAATTATAATG
CATCATCTATTTAGGAGTTAGATTTGGATGTGCTATTGTATGATTACGAA
TAGTCTGTATGTTTCAAGCCCTTCTGTAAAATATGAAGAAAAGTCTCTTA
GCATTCTGTGTAAAACTGTACTGTTAAATATATGTGTGTAATC
RBBP7

SEQ ID NO:117

>gi|13259504|ref|NM_002893.2|Homo sapiens retino-
blastoma binding protein 7 (RBBP7), mRNA
GCCTCGTCAGCTGCCTGGGCGGGCTGGGAGGCGCGGGTTGAAAAGTCTCG
TTCCAAGTTTGGAGAGAGAGAGAAGAGCGCCTCAGACCTCGGTACCCGCG
AGCGGGGAGGAGGCAGGAAAGAAGGACGCGGCGTCTGGGGAGCACCCAGG
CAGCAAGACGGGGCCCGGGCTTTCGACAGTGGGGAGTGTGACGCGCTTGG
GAAAGGCAGGAGCGCCAGCGGTCGGGCTGCTCTTGGCTAACGAGAGGAGT
CCGAGGCGGCGGCGAGGGGCGAACGACCCGACGCAAGATGGCGAGTAAAG
AGATGTTTGAAGATACTGTGGAGGAGCGTGTCATCAATGAAGAATATAAA
ATCTGGAAGAAGAATACACCGTTTCTATATGACCTGGTTATGACCCATGC
TCTTCAGTGGCCCAGTCTTACCGTTCAGTGGCTTCCTGAAGTGACTAAAC
CTGAAGGAAAAGATTATGCCCTTCATTGGCTAGTGCTGGGGACTCATACG
TCTGATGAGCAGAATCATCTGGTGGTTGCTCGAGTACATATTCCCAATGA
TGATGCACAGTTTGATGCTTCCCATTGTGACAGTGACAAGGGTGAATTTG
GTGGCTTTGGTTCTGTAACAGGAAAAATTGAATGTGAAATTAAAATCAAT
CACGAAGGAGAAGTAAACCGTGCTCGTTACATGCCGCAGAATCCTCACAT
CATTGCTACAAAAACACCATCTTCTGATGTGTTGGTTTTTGACTATACAA
AACACCCTGCTAAACCAGACCCAAGTGGAGAATGTAATCCTGATCTCAGA
TTAAGAGGTCACCAGAAGGAAGGCTATGGTCTCTCCTGGAATTCAAATTT
GAGTGGACATCTCCTAAGTGCATCTGATGACCATACTGTTTGTCTGTGGG
ATATAAACGCAGGACCAAAAGAAGGCAAAATTGTGGATGCTAAAGCCATC
TTTACTGGCCACTCAGCTGTTGTAGAGGATGTGGCCTGGCACCTGCTGCA
CGAGTCATTGTTTGGATCTGTTGCTGATGATCAGAAACTTATGATATGGG
ACACCAGGTCCAATACCACCTCCAAGCCGAGTCACTTGGTGGATGCGCAC
ACTGCCGAAGTCAACTGCCTCTCATTCAATCCCTACAGCGAATTTATTCT
AGCCACCGGCTCTGCGGATAAGACCGTAGCTTTATGGGATCTGCGTAACT
TAAAATTAAAACTCCATACCTTCGAATCTCATAAAGATGAAATTTTCCAG
GTCCACTGGTCTCCACATAATGAAACTATTCTGGCTTCAAGTGGTACTGA
CCGCCGCCTGAATGTGTGGGATTTAAGTAAAATTGGGGAAGAACAATCAG
CAGAAGATGCAGAAGATGGGCCTCCAGAACTCCTGTTTATTCATGGAGGA
CACACTGCTAAGATTTCAGATTTTAGCTGGAACCCCAATGAGCCTTGGGT
CATTTGCTCAGTGTCTGAGGATAACATCATGCAGATATGGCAAATGGCTG
AAAATATTTACAATGATGAAGAGTCAGATGTCACGACATCCGAACTGGAG
GGACAAGGATCTTAAACCCAAAGTACGAGAAATGTTTCTGTTGAATGTAA
TGCTACATGAATGCTTGATTTATCAAGCGCCAAAAAGGCATTGTATAGTA
GGAAATGTAAGTGGGGTGGCTTATGGCTTCTTTATCCTCTGATTCTAGCA
CTTTCAAGTGAGCTGTTGCGTACTGTATCATATTGTAGCTATTAGGGAAG
AGAAGAATGTTGCTTAAGAAAGAACATCACCATTGATTTTAAATACAAGT
AGCAGGGTATTGCCTTTGATTCAACTGTTTTAAGTCCTCATTTTCTCAAA
CTAAGTGCTTGCTGTTCCCAAATATGCAAGAATAACTTTTACACTTTTTC
CTTCCAACACTTCTTGATTGGCTTTGCAGAAATAAAGTTTTAAAAT
RBBP4

SEQ ID NO:118

>gi|5032026|ref|NM_005610.1|Homo sapiens retino-
blastoma binding protein 4 (RBBP4), mRNA
CGCGCGCACAGAGCGAGCTCTTGCAGCCTCCCCGCCCCTCCCGCAACGCT
CGACCCCAGGATTCCCCCGGCTCGCCTGCCCGCCATGGCCGACAAGGAAG
CAGCCTTCGACGACGCAGTGGAAGAACGAGTGATCAACGAGGAATACAAA
ATATGGAAAAAGAACACCCCTTTTCTTTATGATTTGGTGATGACCCATGC
TCTGGAGTGGCCCAGCCTAACTGCCCAGTGGCTTCCAGATGTAACCAGAC
CAGAAGGGAAAGATTTCAGCATTCATCGACTTGTCCTGGGGACACACACA
TCGGATGAACAAAACCATCTTGTTATAGCCAGTGTGCAGCTCCCTAATGA
TGATGCTCAGTTTGATGCGTCACACTACGACAGTGAGAAAGGAGAATTTG
GAGGTTTTGGTTCAGTTAGTGGAAAAATTGAAATAGAAATCAAGATCAAC
CATGAAGGAGAAGTAAACAGGGCCCGTTATATGCCCCAGAACCCTTGTAT
CATCGCAACAAAGACTCCTTCCAGTGATGTTCTTGTCTTTGACTATACAA
AACATCCTTCTAAACCAGATCCTTCTGGAGAGTGCAACCCAGACTTGCGT
CTCCGTGGACATCAGAAGGAAGGCTATGGGCTTTCTTGGAACCCAAATCT
CAGTGGGCACTTACTTAGTGCTTCAGATGACCATACCATCTGCCTGTGGG
ACATCAGTGCCGTTCCAAAGGAGGGAAAAGTGGTAGATGCGAAGACCATC
TTTACAGGGCATACGGCAGTAGTAGAAGATGTTTCCTGGCATCTACTCCA
TGAGTCTCTGTTTGGGTCAGTTGCTGATGATCAGAAACTTATGATTTGGG
ATACTCGTTCAAACAATACTTCCAAACCAAGCCACTCAGTTGATGCTCAC
ACTGCTGAAGTGAACTGCCTTTCTTTCAATCCTTATAGTGAGTTCATTCT
TGCCACAGGATCAGCTGACAAGACTGTTGCCTTGTGGGATCTGAGAAATC
TGAAACTTAAGTTGCATTCCTTTGAGTCACATAAGGATGAAATATTCCAG
GTTCAGTGGTCACCTCACAATGAGACTATTTTAGCTTCCAGTGGTACTGA
TCGCAGACTGAATGTCTGGGATTTAAGTAAAATTGGAGAGGAACAATCCC
CAGAAGATGCAGAAGACGGGCCACCAGAGTTGTTGTTTATTCATGGTGGT
CATACTGCCAAGATATCTGATTTCTCCTGGAATCCCAATGAACCTTGGGT
GATTTGTTCTGTATCAGAAGACAATATCATGCAAGTGTGGCAAATGGCAG
AGAACATTTATAATGATGAAGACCCTGAAGGAAGCGTGGATCCAGAAGGA
CAAGGGTCCTAGATATGTCTTTACTTGTTGTGATTTTAGACTCCCCTTTT
TTCTTCTCAACCCTGAGAGTGATTTAACACTGGTTTTGAGACAGACTTTA
TTCAGCTATCCCTCTATATAATAGGTACCACCGATAATGCTATTAGCCCA
AACCGTGGGTTTTTCTAAATATTAATAGGGGGGCTTGATTCAACAAAGCC
ACAGACTTAACGTTGAAATTTTCTTCAGGAATTTTCTAGTAACCCAGGTC
TAAAGTAGCTACAGAAAGGGGAATATTATGTGTGATTATTTTTCTTCTTA
TGCTATATCCCCAAGTTTTTCAGACTCATTTAAGTAAAGGCTAGAGTGAG
TAAGGAATAGAGCCAAATGAGGTAGGTGTCTGAGCCATGAAGTATAAATA
CTGAAAGATGTCACTTTTATTCAGGAAATAGGGGGAGTTCAAGTCGTATA
GATTCCTACTCGAAAATCTTGACACCTGACTTTCCAGGATGCACATTTTC
ATACGTAGACCAGTTTCCTCTTGGTTTCTTCAGTTAAGTCAAAACAACAC
GTTCCTCTTTCCCCATATATTCATATATTTTTGCTCGTTAGTGTATTTCT
TGAGCTGTTTTCATGTTGTTTATTTCCTGTCTGTGAAATGGTGTTTTTTT
TTTTGTTGTTGGTTTTTTTTTTTTTTTTTTAACTTGGGACCACCAAGTTG
TAAAGATGTATGTTTTTACCTGACAGTTATACCACAGGTAGACTGTCAAG
TTGAGAAGAGTGAATCAATAACTTGTATTTGTTTTAAAAATTAAATTAAT
CCTTGATAAGAGTTGCTTTTTTTTTTTAGGAGTTAGTCCTTGACCACTAG
TTTGATGCCATCTCCATTTTGGGTGACCTGTTTCACCAGCAGGCCTGTTA
CTCTCCATGACTAACTGTGTAAGTGCTTAAAATGGAATAAATTGCTTTTC
TACATAAAAAAAAA
RB1

SEQ ID NO:119

gi|4506434|ref|NM_000321.1|Homo sapiens retino-
blastoma 1 (including osteosarcoma) (RB1), mRNA
TTCCGGTTTTTCTCAGGGGACGTTGAAATTATTTTTGTAACGGGAGTCGG
GAGAGGACGGGGCGTGCCCCGCGTGCGCGCGCGTCGTCCTCCCCGGCGCT
CCTCCACAGCTCGCTGGCTCCCGCCGCGGAAAGGCGTCATGCCGCCCAAA
ACCCCCCGAAAAACGGCCGCCACCGCCGCCGCTGCCGCCGCGGAACCCCC
GGCACCGCCGCCGCCGCCCCCTCCTGAGGAGGACCCAGAGCAGGACAGCG
GCCCGGAGGACCTGCCTCTCGTCAGGCTTGAGTTTGAAGAAACAGAAGAA
CCTGATTTTACTGCATTATGTCAGAAATTAAAGATACCAGATCATGTCAG
AGAGAGAGCTTGGTTAACTTGGGAGAAAGTTTCATCTGTGGATGGAGTAT
TGGGAGGTTATATTCAAAAGAAAAAGGAACTGTGGGGAATCTGTATCTTT
ATTGCACGAGTTGACCTAGATGAGATGTCGTTCACTTTACTGAGCTACAG
AAAAACATACGAAATCAGTGTCCATAAATTCTTTAACTTACTAAAAGAAA
TTGATACCAGTACCAAAGTTGATAATGCTATGTCAAGACTGTTGAAGAAG
TATGATGTATTGTTTGCACTCTTCAGCAAATTGGAAAGGACATGTGAACT
TATATATTTGACACAACCCAGCAGTTCGATATCTACTGAAATAAATTCTG
CATTGGTGCTAAAAGTTTCTTGGATCACATTTTTATTAGCTAAAGGGGAA
GTATTACAAATGGAAGATGATCTGGTGATTTCATTTCAGTTAATGCTATG
TGTCCTTGACTATTTTATTAAACTCTCACCTCCCATGTTGCTCAAAGAAC
CATATAAAACAGCTGTTATACCCATTAATGGTTCACCTCGAACACCCAGG
CGAGGTCAGAACAGGAGTGCACGGATAGCAAAACAACTAGAAAATGATAC
AAGAATTATTGAAGTTCTCTGTAAAGAACATGAATGTAATATAGATGAGG
TGAAAAATGTTTATTTCAAAAATTTTATACCTTTTATGAATTCTCTTGGA
CTTGTAACATCTAATGGACTTCCAGAGGTTGAAAATCTTTCTAAACGATA
CGAAGAAATTTATCTTAAAAATAAAGATCTAGATCGAAGATTATTTTTGG
ATCATGATAAAACTCTTCAGACTGATTCTATAGACAGTTTTGAAACACAG
AGAACACCACGAAAAAGTAACCTTGATGAAGAGGTGAATATAATTCCTCC
ACACACTCCAGTTAGGACTGTTATGAACACTATCCAACAATTAATGATGA
TTTTAAATTCTGCAAGTGATCAACCTTCAGAAAATCTGATTTCCTATTTT
AACAACTGCAGAGTGAATCCAAAAGAAAGTATACTGAAAAGAGTGAAGGA
TATAGGATACATCTTTAAAGAGAAATTTGCTAAAGCTGTGGGACAGGGTT
GTGTCGAAATTGGATCACAGCGATACAAACTTGGAGTTCGCTTGTATTAC
CGAGTAATGGAATCCATGCTTAAATCAGAAGAAGAACGATTATCCATTCA
AAATTTTAGCAAACTTCTGAATGACAACATTTTTCATATGTCTTTATTGG
CGTGCGCTCTTGAGGTTGTAATGGCCACATATAGCAGAAGTACATCTCAG
AATCTTGATTCTGGAACAGATTTGTCTTTCCCATGGATTCTGAATGTGCT
TAATTTAAAAGCCTTTGATTTTTACAAAGTGATCGAAAGTTTTATCAAAG
CAGAAGGCAACTTGACAAGAGAAATGATAAAACATTTAGAACGATGTGAA
CATCGAATCATGGAATCCCTTGCATGGCTCTCAGATTCACCTTTATTTGA
TCTTATTAAACAATCAAAGGACCGAGAAGGACCAACTGATCACCTTGAAT
CTGCTTGTCCTCTTAATCTTCCTCTCCAGAATAATCACACTGCAGCAGAT
ATGTATCTTTCTCCTGTAAGATCTCCAAAGAAAAAAGGTTCAACTACGCG
TGTAAATTCTACTGCAAATGCAGAGACACAAGCAACCTCAGCCTTCCAGA
CCCAGAAGCCATTGAAATCTACCTCTCTTTCACTGTTTTATAAAAAAGTG
TATCGGCTAGCCTATCTCCGGCTAAATACACTTTGTGAACGCCTTCTGTG
TGAGCACCCAGAATTAGAACATATCATCTGGACCCTTTTCCAGCACACCC
TGCAGAATGAGTATGAACTCATGAGAGACAGGCATTTGGACCAAATTATG
ATGTGTTCCATGTATGGCATATGCAAAGTGAAGAATATAGACCTTAAATT
CAAAATCATTGTAACAGCATACAAGGATCTTCCTCATGCTGTTCAGGAGA
CATTCAAACGTGTTTTGATCAAAGAAGAGGAGTATGATTCTATTATAGTA
TTCTATAACTCGGTCTTCATGCAGAGACTGAAAACAAATATTTTGCAGTA
TGCTTCCACCAGGCCCCCTACCTTGTCACCAATACCTCACATTCCTCGAA
GCCCTTACAAGTTTCCTAGTTCACCCTTACGGATTCCTGGAGGGAACATC
TATATTTCACCCCTGAAGAGTCCATATAAAATTTCAGAAGGTCTGCCAAC
ACCAACAAAAATGACTCCAAGATCAAGAATCTTAGTATCAATTGGTGAAT
CATTCGGGACTTCTGAGAAGTTCCAGAAAATAAATCAGATGGTATGTAAC
AGCGACCGTGTGCTCAAAAGAAGTGCTGAAGGAAGCAACCCTCCTAAACC
ACTGAAAAAACTACGCTTTGATATTGAAGGATCAGATGAAGCAGATGGAA
GTAAACATCTCCCAGGAGAGTCCAAATTTCAGCAGAAACTGGCAGAAATG
ACTTCTACTCGAACACGAATGCAAAAGCAGAAAATGAATGATAGCATGGA
TACCTCAAACAAGGAAGAGAAATGAGGATCTCAGGACCTTGGTGGACACT
GTGTACACCTCTGGATTCATTGTCTCTCACAGATGTGACTGTATAACTTT
CCCAGGTTCTGTTTATGGCCACATTTAATATCTTCAGCTCTTTTTGTGGA
TATAAAATGTGCAGATGCAATTGTTTGGGTGAGTCCTAAGCCACTTGAAA
TGTTAGTCATTGTTATTTATACAAGATTGAAAATCTTGTGTAAATCCTGC
CATTTAAAAAGTTGTAGCAGATTGTTTCCTCTTCCAAAGTAAAATTGCTG
TGCTTTATGGATAGTAAGAATGGCCCTAGAGTGGGAGTCCTGATAACCCA
GGCCTGTCTGACTACTTTGCCTTCTTTTGTAGCATATAGGTGATGTTTGC
TCTTGTTTTTATTAATTTATATGTATATTTTTTTAATTTAACATGAACAC
CCTTAGAAAATGTGTCCTATCTATCTTCCAAATGCAATTTGATTGACTGC
CCATTCACCAAAATTATCCTGAACTCTTCTGCAAAAATGGATATTATTAG
AAATTAGAAAAAAATTACTAATTTTACACATTAGATTTTATTTTACTATT
GGAATCTGATATACTGTGTGCTTGTTTTATAAAATTTTGCTTTTAATTAA
ATAAAAGCTGGAAGCAAAGTATAACCATATGATACTATCATACTACTGAA
ACAGATTTCATACCTCAGAATGTAAAAGAACTTACTGATTATTTTCTTCA
TCCAACTTATGTTTTTAAATGAGGATTATTGATAGTACTCTTGGTTTTTA
TACCATTCAGATCACTGAATTTATAAAGTACCCATCTAGTACTTGAAAAA
GTAAAGTGTTCTGCCAGATCTTAGGTATAGAGGACCCTAACACAGTATAT
CCCAAGTGCACTTTCTAATGTTTCTGGGTCCTGAAGAATTAAGATACAAA
TTAATTTTACTCCATAAACAGACTGTTAATTATAGGAGCCTTAATTTTTT
TTTCATAGAGATTTGTCTAATTGCATCTCAAAATTATTCTGCCCTCCTTA
ATTTGGGAAGGTTTGTGTTTTCTCTGGAATGGTACATGTCTTCCATGTAT
CTTTTGAACTGGCAATTGTCTATTTATCTTTTATTTTTTTAAGTCAGTAT
GGTCTAACACTGGCATGTTCAAAGCCACATTATTTCTAGTCCAAAATTAC
AAGTAATCAAGGGTCATTATGGGTTAGGCATTAATGTTTCTATCTGATTT
TGTGCAAAAGCTTCAAATTAAAACAGCTGCATTAGAAAAAGAGGCGCTTC
TCCCCTCCCCTACACCTAAAGGTGTATTTAAACTATCTTGTGTGATTAAC
TTATTTAGAGATGCTGTAACTTAAAATAGGGGATATTTAAGGTAGCTTCA
GCTAGCTTTTAGGAAAATCACTTTGTCTAACTCAGAATTATTTTTAAAAA
GAAATCTGGTCTTGTTAGAAAACAAAATTTTATTTTGTGCTCATTTAAGT
TTCAAACTTACTATTTTGACAGTTATTTTGATAACAATGACACTAGAAAA
CTTGACTCCATTTCATCATTGTTTCTGCATGAATATCATACAAATCAGTT
AGTTTTTAGGTCAAGGGCTTACTATTTCTGGGTCTTTTGCTACTAAGTTC
ACATTAGAATTAGTGCCAGAATTTTAGGAACTTCAGAGATCGTGTATTGA
GATTTCTTAAATAATGCTTCAGATATTATTGCTTTATTGCTTTTTTGTAT
TGGTTAAAACTGTACATTTAAAATTGCTATGTTACTATTTTCTACAATTA
ATAGTTTGTCTATTTTAAAATAAATTAGTTGTTAAGAGTC
MEN1

SEQ ID NO:120

>gi|4557744|ref|NM_000244.1|Homo sapiens multiple
endocrine neoplasia I (MEN1), mRNA
GGTGTCCGGAGCCGCGGACCTAGAGATCCCAGAAGCCACAGCGCAGCGGC
CCGGCCCGCCACTATTTCCAGGCTCTGCGGGGCAGGGGCCGCCGCCCACC
GCCCGCCGCCATGGGGCTGAAGGCCGCCCAGAAGACGCTGTTCCCGCTGC
GCTCCATCGACGACGTGGTGCGCCTGTTTGCTGCCGAGCTGGGCCGAGAG
GAGCCGGACCTGGTGCTCCTTTCCTTGGTGCTGGGCTTCGTGGAGCATTT
TCTGGCTGTCAACCGCGTCATCCCTACCAACGTTCCCGAGCTCACCTTCC
AGCCCAGCCCCGCCCCCGACCCGCCTGGCGGCCTCACCTACTTTCCCGTG
GCCGACCTGTCTATCATCGCCGCCCTCTATGCCCGCTTCACCGCCCAGAT
CCGAGGCGCCGTCGACCTGTCCCTCTATCCTCGAGAAGGGGGTGTCTCCA
GCCGTGAGCTGGTGAAGAAGGTCTCCGATGTCATATGGAACAGCCTCAGC
CGCTCCTACTTCAAGGATCGGGCCCACATCCAGTCCCTCTTCAGCTTCAT
CACAGGCACCAAATTGGACAGCTCCGGTGTGGCCTTTGCTGTGGTTGGGG
CCTGCCAGGCCCTGGGTCTCCGGGATGTCCACCTCGCCCTGTCTGAGGAT
CATGCCTGGGTAGTGTTTGGGCCCAATGGGGAGCAGACAGCTGAGGTCAC
CTGGCACGGCAAGGGCAACGAGGACCGCAGGGGCCAGACAGTCAATGCCG
GTGTGGCTGAGCGGAGCTGGCTGTACCTGAAAGGATCATACATGCGCTGT
GACCGCAAGATGGAGGTGGCGTTCATGGTGTGTGCCATCAACCCTTCCAT
TGACCTGCACACCGACTCGCTGGAGCTTCTGCAGCTGCAGCAGAAGCTGC
TCTGGCTGCTCTATGACCTGGGACATCTGGAAAGGTACCCCATGGCCTTA
GGGAACCTGGCAGATCTAGAGGAGCTGGAGCCCACCCCTGGCCGGCCAGA
CCCACTCACCCTCTACCACAAGGGCATTGCCTCAGCCAAGACCTACTATC
GGGATGAACACATCTACCCCTACATGTACCTGGCTGGCTACCACTGTCGC
AACCGCAATGTGCGGGAAGCCCTGCAGGCCTGGGCGGACACGGCCACTGT
CATCCAGGACTACAACTACTGCCGGGAAGACGAGGAGATCTACAAGGAGT
TCTTTGAAGTAGCCAATGATGTCATCCCCAACCTGCTGAAGGAGGCAGCC
AGCTTGCTGGAGGCGGGCGAGGAGCGGCCGGGGGAGCAAAGCCAGGGCAC
CCAGAGCCAAGGTTCCGCCCTCCAGGACCCTGAGTGCTTCGCCCACCTGC
TGCGATTCTACGACGGCATCTGCAAATGGGAGGAGGGCAGTCCCACGCCT
GTGCTGCACGTGGGCTGGGCCACCTTTCTTGTGCAGTCCCTAGGCCGTTT
TGAGGGACAGGTGCGGCAGAAGGTGCGCATAGTGAGCCGAGAGGCCGAGG
CGGCCGAGGCCGAGGAGCCGTGGGGCGAGGAAGCCCGGGAAGGCCGGCGG
CGGGGCCCACGGCGGGAGTCCAAGCCAGAGGAGCCCCCGCCGCCCAAGAA
GCCAGCACTGGACAAGGGCCTGGGCACCGGCCAGGGTGCAGTGTCAGGAC
CCCCCCGGAAGCCTCCTGGGACTGTCGCTGGCACAGCCCGAGGCCCTGAA
GGTGGCAGCACGGCTCAGGTGCCAGCACCCGCAGCATCACCACCGCCGGA
GGGTCCAGTGCTCACTTTCCAGAGTGAGAAGATGAAGGGCATGAAGGAGC
TGCTGGTGGCCACCAAGATCAACTCGAGCGCCATCAAGCTGCAACTCACG
GCACAGTCGCAAGTGCAGATGAAGAAGCAGAAAGTGTCCACCCCTAGTGA
CTACACTCTGTCTTTCCTCAAGCGGCAGCGCAAAGGCCTCTGAACTACTG
GGGACTTCGGACCGCTTGTGGGGACCCAGGCTCCGCCTTAGTCCCCCAAC
TCTGAGCCCATGTTCTGCCCCCAGCCCAAAGGGGACAGGCCTCACCTCTA
CCCAAACCCTAGGTTCCCGGTCCCGAGTACAGTCTGTATCAAACCCACGA
TTTTCTCCAGCTCAGAACCCAGGGCTCTGCCCCAGTCGTTAGAATATAGG
TCTCTTCTCCCAGAATCCCAGCCGGCCAATGGAAACCTCACGCTGGGTCC
TAATTACCAGTCTTTAAAGGCCCAGCCCCTAGAAACCCAAGCTCCTCCTC
GGAACCGCTCACCTAGAGCCAGACCAACGTTACTCAGGGCTCCTCCCAGC
TTGTAGGAGCTGAGGTTTCACCCTTAACCCAAGGGAGCACAGGTCCCACC
TCCAGCCCGGGGAGCCTAGGACCACTCAGCCCCTAGGAGTATATTTCCGC
ACTTCAGAATTCCATATCTTGCGAATCCAAGCTCCCTGCCCCAAATAACT
TCAGTCCTGCTTCCAGAATTTGGAAATCCTAGTTTCCTCTCCTTCGTATC
CCGAGTCTGGGACACAAAACTCCGCCCCCAGCCTATGAGCATCCTGAGCC
CCGCCCTCTTCCTGACGAAACTGGCCCCGGATCAGAGCAGGACCTCCCTT
CCGACCCTCTGGGAACCTCCCAGAGGTCCAGCCCATCTCGGAGCATCCCG
GAGGAAATCTGCAGAGGGGTTAGGAGTGGGTGACAAGAGCCTGATCTCTT
CCTGTTTTGTACATAGATTTATTTTTCAGTTCCAAGAAAGATGAATACAT
TTTGTTAAAAAAAAAAAAAAAA

TABLE 5
Histone Acetyltranferases.
GCN5/PCAP Family.

Gcn5

SEQ ID NO:121

>gi|4503954|ref|NM_001487.1|Homo sapiens GCN5
general control of amino-acid synthesis 5-like 1
(yeast) (GCN5L1), mRNA
CAGCGGGCACGTGACATGGCCCCGGGGAGCCGAGGTGAGCGTCCAGCTTC
CGGAGCCGGAGGGGGCCCGGCGTACCCAGCCCCCAGCCCGACGTGACCAT
GCTGTCCCGCCTCCTAAAAGAACACCAGGCCAAGCAGAATGAACGCAAGG
AGCTGCAGGAAAAGAGGAGGCGAGAGGCTATCACTGCAGCGACCTGCCTG
ACAGAAGCTTTGGTGGATCACCTCAATGTGGGTGTGGCCCAGGCCTACAT
GAACCAGAGAAAGCTGGACCATGAGGTGAAGACCCTACAGGTCCAGGCTG
CCCAATTTGCCAAGCAGACAGGCCAGTGGATCGGAATGGTGGAGAACTTC
AACCAGGCACTCAAGGAAATTGGGGATGTGGAGAACTGGGCTCGGAGCAT
CGAGCTGGACATGCGCACCATTGCCACTGCACTGGAATATGTCTACAAAG
GGCAGCTGCAGTCTGCCCCTTCCTAGCCCCTGTTCCCTCCCCAAACCCTA
TCCCTCCTACCTCACCCGCAGGGGGAAGGAGGGAGGCTGACAAGCCTTGA
ATAAAACACAAGCCTCC
GCN5L2

SEQ ID NO:122

>gi|10835100|ref|NM_021078.1|Homo sapiens GCN5
general control of amino-acid synthesis 5-like 2
(yeast) (GCN5L2), mRNA
CCGCTCTCCGCTGCGGGGGAGGCCATGGCGGAACCTTCCCAGGCCCCGAC
CCCGGCCCCGGCTGCGCAGCCCCGGCCCCTTCAGTCCCCAGCCCCTGCCC
CAACTCCGACTCCTGCACCCAGCCCGGCTTCAGCCCCGATTCCGACTCCC
ACCCCGGCACCAGCCCCTGCCCCAGCTGCAGCCCCAGCCGGCAGCACAGG
GACTGGGGGGCCCGGGGTAGGAAGTGGGGGGGCCGGGAGCGGGGGGGATC
CGGCTCGGCCTGGCCTGAGCCAGCAGCAGCGCGCCAGTCAGAGGAAGGCG
CAAGTCCGGGGGCTGCCCCGCGCCAAGAAGCTTGAGAAGCTAGGGGTCTT
CTCGGCTTGCAAGGCCAATGGAACCTGTAAGTGTAATGGCTGGAAAAACC
CCAAGCCCCCCACTGCACCCCGCATAGATCTGCAGCAGCCAGCTGCCAAC
CTGAGTGAGCTGTGCCGCAGTTGTGAGCACCCCTTGGCTGACCACGTATC
CCACTTGGAGAATGTGTCAGAGGATGAGATAAACCGACTGCTGGGGATGG
TGGTGGATGTGGAGAATCTCTTCATGTCTGTTCACAAGGAAGAGGACACA
GACACCAAGCAGGTCTATTTCTACCTCTTCAAGCTACTGCGGAAATGCAT
CCTGCAGATGACCCGGCCTGTGGTGGAGGGGTCCCTGGGCAGCCCTCCAT
TTGAGAAACCTAATATTGAGCAGGGTGTGCTGAACTTTGTGCAGTACAAG
TTTAGTCACCTGGCTCCCCGGGAGCGGCAGACGATGTTCGAGCTCTCAAA
GATGTTCTTGCTCTGCCTTAACTACTGGGAGCTTGAGACACCTGCCCAGT
TTCGGCAGAGGTCTCAGGCTGAGGACGTGGCTACCTACAAGGTCAATTAC
ACCAGATGGCTCTGTTACTGCCACGTGCCCCAGAGCTGTGATAGCCTCCC
CCGCTACGAAACCACTCATGTCTTTGGGCGAAGCCTTCTCCGGTCCATTT
TCACCGTTACCCGCCGGCAGCTGCTGGAAAAGTTCCGAGTGGAGAAGGAC
AAATTGGTGCCCGAGAAGAGGACCCTCATCCTCACTCACTTCCCCAAATT
CCTGTCCATGCTGGAGGAGGAGATCTATGGGGCAAACTCTCCAATCTGGG
AGTCAGGCTTCACCATGCCACCCTCAGAGGGGACACAGCTGGTTCCCCGG
CCAGCTTCAGTCAGTGCAGCGGTTGTTCCCAGCACCCCCATCTTCAGCCC
CAGCATGGGTGGGGGCAGCAACAGCTCCCTGAGTCTGGATTCTGCAGGGG
CCGAGCCTATGCCAGGCGAGAAGAGGACGCTCCCAGAGAACCTGACCCTG
GAGGATGCCAAGCGGCTCCGTGTGATGGGTGACATCCCCATGGAGCTGGT
CAATGAGGTCATGCTGACCATCACTGACCCTGCTGCCATGCTGGGGCCTG
AGACGAGCCTGCTTTCGGCCAATGCGGCCCGGGATGAGACAGCCCGCCTG
GAGGAGCGCCGCGGCATCATCGAGTTCCATGTCATCGGCAACTCACTGAC
GCCCAAGGCCAACCGGCGGGTGTTGCTGTGGCTCGTGGGGCTGCAGAATG
TCTTTTCCCACCAGCTGCCGCGCATGCCTAAGGAGTATATCGCCCGCCTC
GTCTTTGACCCGAAGCACAAGACTCTGGCCTTGATCAAGGATGGGCGGGT
CATCGGTGGCATCTGCTTCCGCATGTTTCCCACCCAGGGCTTCACGGAGA
TTGTCTTCTGTGCTGTCACCTCGAATGAGCAGGTCAAGGGTTATGGGACC
CACCTGATGAACCACCTGAAGGAGTATCACATCAAGCACAACATTCTCTA
CTTCCTCACCTACGCCGACGAGTACGCCATCGGCTACTTCAAAAAGCAGG
GTTTCTCCAAGGACATCAAGGTGCCCAAGAGCCGCTACCTGGGCTACATC
AAGGACTACGAGGGAGCGACGCTGATGGAGTGTGAGCTGAATCCCCGCAT
CCCCTACACGGAGCTGTCCCACATCATCAAGAAGCAGAAAGAGATCATCA
AGAAGCTGATTGAGCGCAAACAGGCCCAGATCCGCAAGGTCTACCCGGGG
CTCAGCTGCTTCAAGGAGGGCGTGAGGCAGATCCCTGTGGAGAGCGTTCC
TGGCATTCGAGAGACAGGCTGGAAGCCATTGGGGAAGGAGAAGGGGAAGG
AGCTGAAGGACCCCGACCAGCTCTACACAACCCTCAAAAACCTGCTGGCC
CAAATCAAGTCTCACCCCAGTGCCTGGCCCTTCATGGAGCCTGTGAAGAA
GTCGGAGGCCCCTGACTACTACGAGGTCATCCGCTTCCCCATTGACCTGA
AGACCATGACTGAGCGGCTGCGAAGCCGCTACTACGTGACCCGGAAGCTC
TTTGTGGCCGACCTGCAGCGGGTCATCGCCAACTGTCGCGAGTACAACCC
CCCGGACAGCGAGTACTGCCGCTGTGCCAGCGCCCTGGAGAAGTTCTTCT
ACTTCAAGCTCAAGGAGGGAGGCCTCATTGACAAGTAGGCCCATCTTTGG
GCCGCAGCCCTGACCTGGAATGTCTCCACCTCGGATTCTGATCTGATCCT
TAGGGGGTGCCCTGGCCCCACGGACCCGACTCAGCTTGAGACACTCCAGC
CAAGGGTCCTCCGGACCCGATCCTGCAGCTCTTTCTGGACCTTCAGGCAC
CCCCAAGCGTGCAGCTCTGTCCCAGCCTTCACTGTGTGTGAGAGGTCTCC
TGGGTTGGGGCCCAGCCCCTCTAGAGTAGCTGGTGGCCAGGGATGAACCT
TGCCCAGCCGTGGTGGCCCCCAGGCCTGGTCCCCAAGAGCTTTGGAGGCT
TGGATTCCTGGGCCTGGCCCAGGTGGCTGTTTCCCTGAGGACCAGAACTG
CTCATTTTAGCTTGAGTGATGGCTTCAGGGGTTGGAAGTTCAGCCCAAAC
TGAAGGGGGCCATGCCTTGTCCAGCACTGTTCTGTCAGTCTCCCCCAGGG
GTGGGGGGTATGGGGACCATTCATTCCCTGGCATTAATCCCTTAGAGGGA
ATAATAAAGCTTTTTATTTCTCTG
PCAF

SEQ ID NO:123

>gi|6382075|ref|NM_003884.2|Homo sapiens p300/CBP-
associated factor (PCAF), mRNA
GGGGCCGCGTCGACGCGGAAAAGAGGCCGTGGGGGGCCTCCCAGCGCTGG
CAGACACCGTGAGGCTGGCAGCCGCCGGCACGCACACCTAGTCCGCAGTC
CCGAGGAACATGTCCGCAGCCAGGGCGCGGAGCAGAGTCCCGGGCAGGAG
AACCAAGGGAGGGCGTGTGCTGTGGCGGCGGCGGCAGCGGCAGCGGAGCC
GCTAGTCCCCTCCCTCCTGGGGGAGCAGCTGCCGCCGCTGCCGCCGCCGC
CACCACCATCAGCGCGCGGGGCCCGGCCAGAGCGAGCCGGGCGAGCGGCG
CGCTAGGGGGAGGGCGGGGGCGGGGAGGGGGGTGGGCGAAGGGGGCGGGA
GGGCGTGGGGGGAGGGTCTCGCTGTCCCGACTACCAGAGCCCGAGGGAGA
CCCTGGCGGCGGCGGCGGCGCCTGACACTCGGCGCCTCCTGCCGTGCTCC
GGGGCGGCATGTCCGAGGCTGGCGGGGCCGGGCCGGGCGGCTGCGGGGCA
GGAGCCGGGGCAGGGGCCGGGCCCGGGGCGCTGCCCCCGCAGCCTGCGGC
GCTTCCGCCCGCGCCCCCGCAGGGCTCCCCCTGCGCCGCTGCCGCCGGGG
GCTCGGGCGCCTGCGGTCCGGCGACGGCAGTGGCTGCAGCGGGCACGGCC
GAAGGACCGGGAGGCGGTGGCTCGGCCCGAATCGCCGTGAAGAAAGCGCA
ACTACGCTCCGCTCCGCGGGCCAAGAAACTGGAGAAACTCGGAGTGTACT
CCGCCTGCAAGGCCGAGGAGTCTTGTAAATGTAATGGCTGGAAAAACCCT
AACCCCTCACCCACTCCCCCCAGAGCCGACCTGCAGCAAATAATTGTCAG
TCTAACAGAATCCTGTCGGAGTTGTAGCCATGCCCTAGCTGCTCATGTTT
CCCACCTGGAGAATGTGTCAGAGGAAGAAATGAACAGACTCCTGGGAATA
GTATTGGATGTGGAATATCTCTTTACCTGTGTCCACAAGGAAGAAGATGC
AGATACCAAACAAGTTTATTTCTATCTATTTAAGCTCTTGAGAAAGTCTA
TTTTACAAAGAGGAAAACCTGTGGTTGAAGGCTCTTTGGAAAAGAAACCC
CCATTTGAAAAACCTAGCATTGAACAGGGTGTGAATAACTTTGTGCAGTA
CAAATTTAGTCACCTGCCAGCAAAAGAAAGGCAAACAATAGTTGAGTTGG
CAAAAATGTTCCTAAACCGCATCAACTATTGGCATCTGGAGGCACCATCT
CAACGAAGACTGCGATCTCCCAATGATGATATTTCTGGATACAAAGAGAA
CTACACAAGGTGGCTGTGTTACTGCAACGTGCCACAGTTCTGCGACAGTC
TACCTCGGTACGAAACCACACAGGTGTTTGGGAGAACATTGCTTCGCTCG
GTCTTCACTGTTATGAGGCGACAACTCCTGGAACAAGCAAGACAGGAAAA
AGATAAACTGCCTCTTGAAAAACGAACTCTAATCCTCACTCATTTCCCAA
AATTTCTGTCCATGCTAGAAGAAGAAGTATATAGTCAAAACTCTCCCATC
TGGGATCAGGATTTTCTCTCAGCCTCTTCCAGAACCAGCCAGCTAGGCAT
CCAAACAGTTATCAATCCACCTCCTGTGGCTGGGACAATTTCATACAATT
CAACCTCATCTTCCCTTGAGCAGCCAAACGCAGGGAGCAGCAGTCCTGCC
TGCAAAGCCTCTTCTGGACTTGAGGCAAACCCAGGAGAAAAGAGGAAAAT
GACTGATTCTCATGTTCTGGAGGAGGCCAAGAAACCCCGAGTTATGGGGG
ATATTCCGATGGAATTAATCAACGAGGTTATGTCTACCATCACGGACCCT
GCAGCAATGCTTGGACCAGAGACCAATTTTCTGTCAGCACACTCGGCCAG
GGATGAGGCGGCAAGGTTGGAAGAGCGCAGGGGTGTAATTGAATTTCACG
TGGTTGGCAATTCCCTCAACCAGAAACCAAACAAGAAGATCCTGATGTGG
CTGGTTGGCCTACAGAACGTTTTCTCCCACCAGCTGCCCCGAATGCCAAA
AGAATACATCACACGGCTCGTCTTTGACCCGAAACACAAAACCCTTGCTT
TAATTAAAGATGGCCGTGTTATTGGTGGTATCTGTTTCCGTATGTTCCCA
TCTCAAGGATTCACAGAGATTGTCTTCTGTGCTGTAACCTCAAATGAGCA
AGTCAAGGGCTATGGAACACACCTGATGAATCATTTGAAAGAATATCACA
TAAAGCATGACATCCTGAACTTCCTCACATATGCAGATGAATATGCAATT
GGATACTTTAAGAAACAGGGTTTCTCCAAAGAAATTAAAATACCTAAAAC
CAAATATGTTGGCTATATCAAGGATTATGAAGGAGCCACTTTAATGGGAT
GTGAGCTAAATCCACGGATCCCGTACACAGAATTTTCTGTCATCATTAAA
AAGCAGAAGGAGATAATTAAAAAACTGATTGAAAGAAAACAGGCACAAAT
TCGAAAAGTTTACCCTGGACTTTCATGTTTTAAAGATGGAGTTCGACAGA
TTCCTATAGAAAGCATTCCTGGAATTAGAGAGACAGGCTGGAAACCGAGT
GGAAAAGAGAAAAGTAAAGAGCCCAGAGACCCTGACCAGCTTTACAGCAC
GCTCAAGAGCATCCTCCAGCAGGTGAAGAGCCATCAAAGCGCTTGGCCCT
TCATGGAACCTGTGAAGAGAACAGAAGCTCCAGGATATTATGAAGTTATA
AGGTTCCCCATGGATCTGAAAACCATGAGTGAACGCCTCAAGAATAGGTA
CTACGTGTCTAAGAAATTATTCATGGCAGACTTACAGCGAGTCTTTACCA
ATTGCAAAGAGTACAACGCCGCTGAGAGTGAATACTACAAATGTGCCAAT
ATCCTGGAGAAATTCTTCTTCAGTAAAATTAAGGAAGCTGGATTAATTGA
CAAGTGATTTTTTTTCCCCCTCTGCTTCTTAGAAACTCACCAAGCAGTGT
GCCTAAAGCAAGGT
MOZ

SEQ ID NO:124

>gi|5803097|ref|NM_006766.1|Homo sapiens zinc
finger protein 220 (ZNF220), mRNA
GGCACGAGGTTTGGGGCATCTCCGCGGTCCGGCCCGGGGCCCCGGGATCT
CGGCTGTCCTTCCTCCCGGCATAAGATGCACATTTTTCTGCTCTGGAGCC
GGGAATGAAATATTCTTGAGTTCTTACAACTTTATGACGAGACCCATGTG
TGGTGCTATTGAGAAATTCATTGGGAAGTTGGAAGACATTTCAAACAACA
GGTTGTTTTGGTTTCTATAGTACAATTGGGGTGGCATTCTGTTTTGTGAA
AGGAGGAAGGACTTAGGCCAGAAAACTCATATGCTATGGTTAACTGGTTC
CCAGCCTCCGAGAATCTTGTTTTCCATGGTGTAAAACTTACTCAGCATCA
GGATAAGGGATAACGACTCTATGGATATACAGAATCCTTCACCATGGTAA
AACTCGCAAACCCGCTTTATACTGAGTGGATTTTGGAGGCCATCAAAAAA
GTGAAAAAGCAGAAACAGCGTCCTTCAGAAGAAAGGATATGCAATGCTGT
GTCTTCATCCCATGGCTTGGATCGTAAAACTGTTTTAGAACAATTGGAGT
TGAGTGTTAAAGATGGAACAATTTTAAAAGTCTCAAATAAAGGACTCAAT
TCCTATAAAGATCCTGATAATCCTGGGCGAATAGCACTTCCTAAGCCTCG
GAACCATGGAAAATTGGATAATAAACAAAATGTGGATTGGAATAAACTGA
TAAAGCGGGCAGTTGAGGGCTTGGCAGAGTCTGGTGGCTCAACTTTGAAA
AGCATTGAACGTTTTTTGAAAGGTCAGAAGGATGTGTCTGCATTATTCGG
AGGCAGTGCTGCCTCTGGCTTTCACCAGCAGTTACGATTGGCTATCAAAC
GTGCCATTGGCCACGGCAGACTCCTTAAAGATGGACCTCTTTATCGGCTC
AACACTAAAGCAACCAACGTGGATGGGAAAGAGAGTTGTGAGTCTCTTTC
CTGTTTACCTCCAGTGTCCCTTCTTCCACATGAAAAGGATAAGCCGGTTG
CTGAACCAATCCCCATCTGTAGTTTCTGTCTTGGTACAAAAGAACAAAAC
CGAGAAAAGAAGCCAGAGGAACTCATCTCCTGTGCCGACTGTGGCAACAG
TGGCCATCCATCCTGTTTAAAGTTTTCCCCTGAACTAACGGTTCGAGTGA
AGGCCTTACGGTGGCAGTGCATCGAGTGTAAAACATGCAGCTCCTGTCGA
GATCAAGGCAAAAATGCGGATAACATGCTCTTTTGTGATTCATGTGACCG
AGGTTTTCACATGGAGTGTTGTGATCCGCCACTCACCCGTATGCCAAAAG
GCATGTGGATATGTCAAATATGTCGACCTAGGAAAAAAGGACGAAAACTT
CTACAAAAGAAGGCAGCACAGATAAAACGGCGCTATACTAATCCAATAGG
ACGTCCAAAAAACAGGTTAAAGAAACAAAACACGGTATCAAAAGGTCCCT
TCAGCAAAGTTCGAACTGGCCCTGGAAGGGGTAGGAAACGAAAAATCACT
CTTTCCAGCCAATCAGCATCATCATCATCAGAAGAAGGATATTTAGAGCG
GATAGATGGCTTGGACTTCTGCAGAGATAGCAATGTGTCCTTGAGGTTCA
ACAAGAAAACCAAAGGGCTCATTGATGGCCTTACCAAATTTTTTACCCCT
TCCCCTGATGGGCGGAAAGCTCGGGGGGAAGTGGTGGACTACTCTGAGCA
ATATCGAATCAGAAAGAGGGGCAACAGGAAATCAAGCACTTCAGATTGGC
CCACAGACAATCAGGATGGCTGGGATGGCAAACAAGAAAATGAGGAGCGA
CTTTTTGGGAGCCAGGAAATCATGACTGAGAAAGATATGGAATTATTTCG
TGATATCCAAGAACAAGCACTGCAGAAAGTTGGAGTGACTGGTCCCCCTG
ATCCACAGGTCCGCTGTCCCTCTGTCATTGAGTTTGGGAAGTATGAAATT
CACACCTGGTACTCCTCCCCATATCCTCAAGAATACTCAAGGCTGCCCAA
ATTGTATCTTTGTGAATTTTGTCTAAAATATATGAAAAGTAGAACTATTC
TGCAGCAGCACATGAAGAAATGTGGTTGGTTCCATCCTCCTGCCAATGAG
ATTTACAGAAAGAATAATATTTCTGTCTTTGAGGTTGATGGGAATGTGAG
TACCATTTATTGTCAAAACCTGTGTCTTTTGGCAAAGTTGTTTCTTGACC
ACAAAACCCTCTATTACGATGTGGAGCCATTTCTTTTTTATGTACTAACA
CAGAATGATGTCAAGGGCTGCCACCTTGTTGGCTACTTTTCTAAGGAAAA
GCACTGCCAACAGAAGTACAATGTTTCCTGTATAATGATTCTTCCTCAAT
ACCAGCGTAAGGGCTATGGCAGGTTTCTCATCGATTTCAGTTATTTGTTA
TCAAAGCGTGAAGGCCAAGCAGGGTCTCCAGAGAAACCGTTATCTGATCT
GGGTCGTCTTTCCTACATGGCATATTGGAAAAGTGTAATATTGGAGTGCC
TTTATCACCAAAATGACAAGCAGATCAGCATTAAGAAGTTAAGCAAGTTG
ACTGGAATCTGCCCTCAAGACATCACTTCCACACTCCACCACCTACGAAT
GCTGGACTTCCGTAGTGACCAATTTGTGATTATCCGCCGGGAAAAACTTA
TCCAGGATCACATGGCAAAGCTTCAGCTGAATTTGCGACCTGTAGATGTA
GATCCAGAATGTTTGCGCTGGACTCCAGTCATAGTGTCCAACTCTGTGGT
CTCAGAGGAGGAAGAAGAGGAGGCTGAGGAAGGAGAAAACGAAGAGCCAC
AGTGCCAGGAAAGAGAATTAGAGATCAGTGTGGGAAAGTCTGTGTCTCAT
GAGAACAAAGAACAAGATTCTTATTCAGTAGAAAGTGAAAAGAAACCAGA
AGTTATGGCTCCAGTCAGTTCTACACGTTTGAGCAAACAAGTCCTTCCTC
ATGATAGTCTTCCTGCAAATAGCCAGCCATCTCGGAGGGGCCGCTGGGGG
AGGAAGAACAGAAAAACCCAGGAACGTTTTGGTGATAAAGATTCTAAACT
GCTCTTGGAAGAGACGTCTTCAGCTCCTCAGGAACAATATGGAGAATGTG
GGGAGAAATCAGAAGCCACCCAGGAACAATACACTGAAAGTGAAGAACAG
CTGGTGGCTTCTGAGGAGCAGCCAAGCCAGGACGGGAAACCTGACCTTCC
CAAGAGAAGACTCAGTGAGGGGGTTGAGCCCTGGCGAGGACAGCTCAAGA
AAAGCCCTGAGGCTCTGAAGTGCAGATTAACAGAAGGAAGTGAGAGGCTG
CCCCGTCGCTACAGTGAGGGTGACAGGGCTGTCCTCAGGGGCTTCAGTGA
GAGCAGCGAGGAGGAGGAGGAGCCGGAAAGCCCTCGGTCAAGCTCGCCAC
CAATTCTCACAAAGCCCACGCTGAAGCGAAAGAAACCATTTCTCCACCGA
AGGAGGAGAGTCCGAAAGCGCAAACACCACAATAGCAGTGTAGTCACAGA
AACTATTTCTGAGACCACTGAAGTGTTAGATGAACCTTTTGAAGATTCTG
ACTCCGAGAGGCCAATGCCAAGATTAGAACCCACATTTGAGATCGATGAA
GAAGAGGAGGAAGAGGATGAAAATGAACTTTTCCCTAGAGAATACTTCCG
TCGTTTGTCTTCGCAGGATGTACTCAGGTGTCAGTCCTCTTCTAAGAGGA
AGTCTAAAGATGAAGAAGAAGATGAAGAGTCAGATGATGCTGATGACACT
CCTATCTTAAAGCCAGTATCTCTTTTGCGAAAACGTGATGTGAAGAATTC
TCCTCTTGAGCCAGATACATCCACACCTTTGAAAAAGAAAAAGGGATGGC
CCAAAGGCAAGAGCCGCAAACCAATCCACTGGAAGAAAAGACCTGGTCGA
AAACCAGGATTTAAGTTGAGTCGGGAAATCATGCCAGTTTCTACTCAAGC
ATGCGTCATTGAGCCCATCGTTTCCATTCCTAAAGCTGGACGTAAACCCA
AGATCCAGGAGAGTGAAGAAACTGTTGAGCCAAAAGAAGACATGCCCCTA
CCCGAGGAGAGGAAGGAGGAGGAGGAGATGCAAGCAGAGGCAGAAGAGGC
TGAAGAGGGTGAGGAAGAGGATGCAGCCAGCAGTGAAGTCCCAGCAGCCT
CTCCAGCAGACAGCAGCAATAGTCCTGAGACCGAAACCAAGGAGCCTGAG
GTGGAGGAGGAAGAAGAGAAGCCCCGTGTCTCAGAGGAGCAGAGGCAGTC
AGAGGAGGAGCAGCAGGAATTAGAGGAGCCAGAGCCAGAGGAGGAGGAAG
ATGCAGCTGCAGAGACTGCCCAGAATGACGACCACGACGCTGATGATGAG
GATGATGGCCACCTGGAGTCCACAAAGAAAAAGGAGCTAGAGGAACAGCC
CACGAGGGAAGATGTCAAGGAGGAGCCTGGTGTTCAAGAGTCTTTTTTAG
ATGCTAATATGCAGAAGAGTAGGGAAAAGATAAAGGATAAAGAGGAAACC
GAGCTGGATTCCGAAGAGGAGCAGCCTTCCCATGACACGTCCGTGGTGTC
AGAGCAGATGGCTGGGTCTGAGGACGACCACGAAGAAGACTCCCACACTA
AGGAAGAGTTAATCGAATTAAAAGAGGAGGAAGAGATTCCTCATAGTGAG
CTGGATCTGGAAACTGTACAGGCAGTGCAGTCTTTGACTCAAGAAGAAAG
CAGTGAGCATGAGGGCGCCTACCAGGACTGTGAGGAAACTCTTGCGGCGT
GTCAGACCCTGCAGAGTTACACCCAGGCTGACGAGGACCCTCAGATGTCC
ATGGTTGAAGACTGTCATGCGTCAGAACATAATAGCCCTATCTCCTCCGT
TCAGTCTCACCCCAGCCAGTCAGTCCGTTCGGTCAGCAGTCCCAACGTGC
CTGCCCTTGAGAGTGGCTACACCCAGATCAGCCCAGAACAAGGATCCCTG
TCCGCACCCTCTATGCAGAACATGGAGACCAGCCCCATGATGGATGTGCC
TTCCGTATCAGACCACTCTCAGCAGGTGGTGGACAGCGGCTTCAGTGACC
TGGGCAGCATTGAGAGCACCACTGAAAACTATGAGAACCCAAGCAGTTAC
GACTCCACGATGGGCGGCAGCATCTGTGGGAACAGCTCTTCCCAGAGCAG
CTGCTCCTACGGTGGGCTGTCGTCCTCCAGCAGCCTCACCCAGAGCAGCT
GTGTGGTCACTCAGCAGATGGCCAGCATGGGCAGCAGCTGCAGCATGATG
CAGCAGAGCAGCGTCCAGCCTGCTGCCAACTGCAGCATCAAGTCACCTCA
GAGCTGCGTGGTGGAGAGGCCTCCCAGTAACCAGCAGCAGCAGCCGCCAC
CACCGCCTCCACAGCAGCCACAGCCGCCGCCGCCACAACCACAACCAGCA
CCACAGCCTCCACCACCCCAGCAGCAGCCGCAACAGCAGCCGCAGCCTCA
GCCCCAGCAGCCTCCACCCCCACCCCCTCCCCAGCAGCAGCCCCCGCTGT
CACAGTGTAGTATGAATAACAGTTTCACCCCAGCTCCTATGATCATGGAG
ATACCAGAATCTGGAAGCACTGGGAACATAAGTATCTATGAGAGGATTCC
AGGGGATTTTGGTGCCGGCAGCTACTCTCAACCATCAGCCACCTTCAGCC
TAGCCAAGCTGCAGCAGCTGACCAACACCATTATGGACCCTCATGCCATG
CCTTATAGCCATTCTCCTGCTGTGACTTCCTATGCAACCAGTGTTTCTCT
GTCCAATACAGGACTGGCTCAGCTGGCTCCATCTCATCCCTTAGCTGGGA
CTCCTCAAGCACAAGCCACCATGACGCCACCCCCAAACTTGGCATCCACT
ACCATGAACCTCACATCTCCTCTGCTTCAGTGCAACATGTCTGCCACCAA
CATTGGCATTCCTCACACGCAGAGATTGCAAGGGCAAATGCCAGTGAAGG
GGCACATTTCCATCCGCTCCAAGTCTGCGCCACTGCCCTCTGCGGCTGCT
CACCAGCAGCAGCTGTATGGCCGTAGCCCATCGGCAGTTGCCATGCAGGC
TGGCCCTCGCGCACTGGCTGTTCAGCGTGGCATGAACATGGGGGTTAATC
TGATGCCTACTCCCGCCTATAATGTCAATTCCATGAATATGAACACCTTG
AATGCCATGAACAGCTATCGAATGACACAGCCCATGATGAACAGCAGTTA
CCATAGTAACCCTGCCTACATGAACCAGACAGCACAGTATCCTATGCAGA
TGCAGATGGGAATGATGGGGAGCCAGGCCTATACCCAGCAGCCTATGCAG
CCTAACCCTCATGGGAACATGATGTACACAGGCCCCTCCCATCACAGCTA
CATGAACGCTGCTGGCGTGCCCAAGCAGTCACTCAACGGACCTTACATGA
GAAGATGAGCAAGATGAACTTGCAATCAAAAACTTAAATATATATAAATA
AAGGAACCTTTTATACTGACAAACCAGAGAAAAATGGACCTTTTTCCAGT
TAAAATATTGCTGTAGATTTAGAGGAATTTTTCTTTGGTTTATTTTATTT
TTTAGAAAACCTGATCTTCTCTTTTTTTTGGGTTCATTTTGTTGTGGGTT
TTGGTTTTCTTCACAATCTTGAACATTTTACAGTAGAACTCATCTAAAAA
TGGATTTGGGGATGGGGAAACATGCACAAAATCTTTTCATAATTAAAAAG
AGCCTTACTTTCTTTACATACCACATGGACAGAATTTGTGTAAAAGTGAA
TTATCTTTATTTTAAAATGTATGTTTCCCCTCACTGTTTGCAGCTCCCAA
TGTTGTCATTTTTAAATGTTATATACATCTCAAGGGTTAACCAGACCCTT
TCCTCCAAACCCAACCTTTCATTTCCTACTTCATTCCAGCAGGAGGCACT
TAGGGGAGACTCGGATGGGGACATGGAGAACAACCCAAGCTCCTTAAACT
TATTATTATTGTTAATATTATTATTATTATTATTAATAAAGTGAGGCAGG
AAAATGCTTCTCCTTTTAAAATCCCCTCCACTCCTCACACACACACACCT
CTTGAAACCCTTCCCCAAGAATGTTTCTTTATAGACGGACTTCATTGAAA
TCTTTGTTGTTCTTGAATCAAGTGTAATATAATTTTTTTCTTCTTTTTTA
AAATATTCCCACTCAGCACTCAGAGACACAAAAATACTGTAAGTCTCAAT
TAACAGCAGAATCTCAGAGAAAAGCTGTTTGCAATCCAAATCCAGCCTTT
GGAGGAATAGAGATGGTCAATTAACAATCAAAAAGAGGAGATTAACCTCT
TGTTTTTTTACCACCTGGTGAATCAGCCATAACGCACACACACGCCACCC
AGCCTCTTGTTTCTAGTATGTACTTTGAAATGCTAACTGAGGGTCTTGAT
GCTTGAGCCTTTGACTGATAAAACTCAAATAGCAGTCCCCAGTGATTTGC
CTCTTAGGTTCTTTCTTAAATTGTTGGTGGATGACTGTACATTTTAGTGA
TTTGAAAAATAACTGACAAACCATTGAAACAGTTTATTTTATGTTGGAAG
AGATGGCGCAGATGTGTGTCAGAAGGGAGATCACGGTGTGAGTTTCGTAG
CTATTTAAGTGATACATACCTCTAGTTTTTGTATGTCTTTTGAGATCCTG
AGTTCATCCCCTGTGAATCAGAGTGCACAAGCACCTCTCCTGTGAGTGGC
TAATGAGAAGAGGGACAGACCGACCACCAGCACAGTAGGGCAGATCTGGA
CAGCAGAATGTTATAACGCAAGTTCATGTGTTGCTCCCAACTCCATTCTC
TTTTCTCTCGTGCAACCAGTTTGCCCATTCTCTTCCTATTACTTGCTCCA
GGGATAGGTAAAAAAAAAA
HBO1

SEQ ID NO:125

>gi|5901961|ref|NM_007067.1|Homo sapiens histone
acetyltransferase (HBOA), mRNA
GCCGCTGCCCGAATCGGAACCGTCGGGCCGCAGCCGCCGGCAATGCCGCG
AAGGAAGAGGAATGCAGGCAGTAGTTCAGATGGAACCGAAGATTCCGATT
TTTCTACAGATCTCGAGCACACAGACAGTTCAGAAAGTGATGGCACATCC
CGACGATCTGCTCGAGTCACCCGCTCCTCAGCCAGGCTAAGCCAGAGTTC
TCAAGATTCCAGTCCTGTTCGAAATCTGCAGTCTTTTGGCACTGAGGAGC
CTGCTTACTCTACCAGAAGAGTGACCCGTAGTCAGCAGCAGCCTACCCCA
GTGACACCGAAAAAATACCCTCTTCGGCAGACTCGTTCATCTGGTTCAGA
AACTGAGCAAGTGGTTGATTTTTCAGATAGAGAAACTAAAAATACAGCTG
ATCATGATGAGTCACCGCCTCGAACTCCAACTGGAAATGCGCCTTCTTCT
GAGTCTGACATAGATATCTCCAGCCCCAATGTATCTCACGATGAGAGCAT
TGCCAAGGACATGTCCCTGAAGGACTCAGGCAGTGATCTCTCTCATCGCC
CCAAGCGCCGTCGCTTCCATGAAAGCTACAACTTCAATATGAAGTGTCCT
ACACCAGGCTGTAACTCTCTAGGACACCTTACAGGAAAACATGAGAGACA
TTTCTCCATCTCAGGATGCCCACTGTATCATAACCTCTCAGCTGACGAAT
GCAAGGTGAGAGCACAGAGCCGGGATAAGCAGATAGAAGAAAGGATGCTG
TCTCACAGGCAAGATGACAACAACAGGCATGCAACCAGGCACCAGGCACC
AACGGAGAGGCAGCTTCGATATAAGGAAAAAGTGGCTGAACTCAGGAAGA
AAAGAAATTCTGGACTGAGCAAAGAACAGAAAGAGAAATATATGGAACAC
AGACAGACCTATGGGAACACACGGGAACCTCTTTTAGAAAACCTGACAAG
CGAGTATGACTTGGATCTTTTCCGAAGAGCACAAGCCCGGGCTTCAGAGG
ATTTGGAGAAGTTAAGGCTGCAAGGCCAAATCACAGAGGGAAGCAACATG
ATTAAAACAATTGCTTTTGGCCGCTATGAGCTTGATACCTGGTATCATTC
TCCATATCCTGAAGAATATGCACGGCTGGGACGTCTCTATATGTGTGAAT
TCTGTTTAAAATATATGAAGAGCCAAACGATACTCCGCCGGCACATGGCC
AAATGTGTGTGGAAACACCCACCTGGTGATGAGATATATCGCAAAGGTTC
AATCTCTGTGTTTGAAGTGGATGGCAAGAAAAACAAGATCTACTGCCAAA
ACCTGTGCCTGTTGGCCAAACTTTTTCTGGACCACAAGACATTATATTAT
GATGTGGAGCCCTTCCTGTTCTATGTTATGACAGAGGCGGACAACACTGG
CTGTCACCTGATTGGATATTTTTCTAAGGAAAAGAATTCATTCCTCAACT
ACAACGTCTCCTGTATCCTTACTATGCCTCAGTACATGAGACAGGGCTAT
GGCAAGATGCTTATTGATTTCAGTTATTTGCTTTCCAAAGTCGAAGAAAA
AGTTGGCTCCCCAGAACGTCCACTCTCAGATCTGGGGCTTATAAGCTATC
GCAGTTACTGGAAAGAAGTACTTCTCCGCTACCTGCATAATTTTCAAGGC
AAAGAGATTTCTATCAAAGAAATCAGTCAGGAGACGGCTGTGAATCCTGT
GGACATTGTCAGCACTCTGCAAGCCCTTCAGATGCTCAAATACTGGAAGG
GAAAACACCTAGTTTTAAAGAGACAGGACCTGATTGATGAGTGGATAGCC
AAAGAGGCCAAAAGGTCCAACTCCAATAAAACCATGGATCCCAGCTGCTT
AAAATGGACCCCTCCCAAGGGCACTTAAAGTGACCTGTCATTCCGAGCCA
GCGAACCCCAGCAGTAGGAATCCGTACCCTAGGGATCTGTCTGTCATTTC
TCTGTTGCTCTTGTGATTGGCAAGTACAGTATCCTTTGGGAAGGCCATCC
CCCTCAGGACTGTCCTGGCTCCGACCTTTGTGTACACTGCAGACGCTGGT
TCTGAGGAACTGTTGTTTCGGCCTCAGTGAGGTTGCCTGGATGGGATCTG
TATTAGACTTGAGTGCAGGTCTCTCAGCACTGACCCAAGGAGTTCTGTTA
TGGTACTGTACCTGTCCAGTCACTGGTTCTCTCCTCATGTCCTCTCGCCC
CATGAGGTTGTGTTGTGTCTTCTAAGCGTGGTACTAGTGCTTGCCACCTG
GTCACCAGACCTCCAAATATGGCTGCCACCACCAGGACCTTTCCAGTTAC
TCCTTATATGTGTGTTCTATGGAGGGGCAGGGAAAAGGTGGCACTTGTGA
GTGTGTGTGGATTGGCAGGGGGTCCATTCACTTTGGGTTCCATCTTGCTT
TAAATTTCTTCATTTTGATTAAGAGACCTCTTTTTGATCTGTATTGGGCT
AACCAGAGCCAAATACTTTTGAAGAGTTTCCCAGGGACTAGTCATGGTAA
TAGCATATAATTGATCTGAATGAGATGGAGAGAAGAATGAAGGGGTGGTG
GTTCTGGGTTTGATTTGAGTTCACCTGTGGGCAGTGGGCAGTGGGCAGTG
TCTTGGTGAAAGGGAACGGATACTACTTTTTGCCTCACCGTAAAGTACTC
ACTAGTAAATATTTCCTTCTCTCTTTACTCCCACTTTTTACGTTTGCAGG
TGCCAAAGTAATGTCCACTTTTCCCTTTCATGCTGCATATTAACTGGTTA
ATTATACTGCAGAAACCTTTTCACCTCCACTAGTCTGATACAGTACATCT
GTACTTCCATATACCTTGCACTGATTTTGTCTGAGTGCCCTGGGAGAAGT
AGAAAATGATTGAAAGTGACTTCCGTATCTCAGCCCATGACTCAGCAAGG
CAGAATGGCCACCCCTGCCAAAGTTTGCTTCTCTTTTCAACAGTGCCTCA
CCCTCCCTCTAGGATTAAAGTGCTTCTGCCCTTCCACGAACTCCTCCTCC
ATTTCCTTTTTGGGATTTGTCACCATCCTTCTATTCTCTGGTCTTCTATT
TTTGGTGTTGTTCAAGTGAAGGAAGAGATGTTCCCTCTAATTTCTCTCTA
GCCCATTATAACCTGCTATCTTGGGGCAACTTTTGATGTATGACATGTCA
CCCTTCCCAACTTGGTCTCCTCCAACATGCTGTCTTCATGTGGAGCCCTC
ACCACAATCCCTGACTCCGGTCATTTGTGCCTTTCTCTTGTCATCTCTGT
ACACTACTTATATTCACTGTGGGTTGGGGGAGCTAATTTTAAGCATGTTC
AGTGGCAGCTCCCCTCCAGTTTCAGTGTCACTGTTAAAATTTATCAAAAA
GCAACTTCACTAGGGGTTTTCTTAAGGGATAAAGGCCTTTTACAGAAGCT
AAACCCTTCCCCACATGTGGTAGAATGTGCTCTTCTATATCTACTCCTCA
ATAAAGCATGTTCTCTGCTCAAAAAAAAAAAAAAAAAAAAAAAAAAAAAA
AAAA

SRC Family

SRC-1

SEQ ID NO:126

>gi|4507198|ref|NM_003743.1|Homo sapiens nuclear
receptor coactivator 1 (NCOA1), mRNA
ATATCATCGACAGGGAGCACAGTGGGCTTTCTCCTCAAGATGACACTAAT
TCTGGAATGTCAATTCCCCGAGTAAATCCCTCGGTCAATCCTAGTATCTC
TCCAGCTCATGGTGTGGCTCGTTCATCCACATTGCCACCATCCAACAGCA
ACATGGTATCCACCAGAATAAACCGCCAGCAGAGCTCAGACCTTCATAGC
AGCAGTCATAGTAATTCTAGCAACAGCCAAGGAAGTTTCGGATGCTCACC
CGGAAGTCAGATTGTAGCCAATGTTGCCTTAAACAAAGGACAGGCCAGTT
CACAGAGCAGTAAACCCTCTTTAAACCTCAATAATCCTCCTATGGAAGGT
ACAGGAATATCCCTAGCACAGTTCATGTCTCCAAGGAGACAGGTTACTTC
TGGATTGGCAACAAGGCCCAGGATGCCAAACAATTCCTTTCCTCCTAATA
TTTCGACATTAAGCTCTCCCGTTGGCATGACAAGTAGTGCCTGTAATAAT
AATAACCGATCTTATTCAAACATCCCAGTAACATCTTTACAGGGTATGAA
TGAAGGACCCAATAACTCCGTTGGCTTCTCTGCCAGTTCTCCAGTCCTCA
GGCAGATGAGCTCACAGAATTCACCTAGCAGATTAAATATACAACCAGCA
AAAGCTGAGTCCAAAGATAACAAAGAGATTGCCTCAACTTTAAATGAAAT
GATTCAATCTGACAACAGCTCTAGTGATGGCAAACCTCTGGATTCAGGGC
TTCTGCATAACAATGACAGACTTTCAGATGGAGACAGTAAATACTCTCAA
ACCAGTCACAAACTAGTGCAGCTTTTGACAACAACTGCCGAACAGCAGTT
ACGGCATGCTGATATAGACACAAGCTGCAAAGATGTCCTGTCTTGCACAG
GCACTTCCAACTCTGCCTCTGCTAACTCTTCAGGAGGTTCTTGTCCCTCT
TCTCATAGCTCATTGACAGCACGGCATAAAATTCTACACCGGCTCTTACA
GGAGGGTAGCCCCTCAGATATCACCACTTTGTCTGTCGAGCCTGATAAAA
AGGACAGTGCATCTACTTCTGTGTCAGTGACTGGACAGGTACAAGGAAAC
TCCAGTATAAAACTAGAACTGGATGCTTCAAAGAAAAAAGAATCAAAAGA
CCATCAGCTCCTACGCTATCTTTTAGATAAAGATGAGAAAGATTTAAGAT
CACCTCCAAACCTGAGCCTGGATGATGTAAAGGTGAAAGTGGAAAGAAAA
GAACAGATGGATCCATGTAATACAAACCCAACCCCAATGACGAAGGCCAC
TCCTGAGGAAATAAAACTGGAGGCCCAGAGCCAGTTTACAGCTGACCTTG
ACCAGTTTGATCAGTTACTGCCCACGCTGGAGAAGGCAGCACAGTTGCCA
GGCTTATGTGAGACAGACAGGATGGATGGTGCGGTCACCAGTGTAACCAT
CAAATCGGAGATCCTGCCAGCTTCACTTCAGTCCGCCACTGCCAGACCCA
CTTCCAGGCTGAATAGATTACCTGAGCTGGAATTGGAAGCAATTGATAAC
CAATTTGGACAACCAGGAACAGGCGATCAGATTCCATGGACAAATAATAC
AGTGACAGCTATAAATCAGAGTAAATCAGAAGACCAGTGTATTAGCTCAC
AATTAGATGAGCTTCTCTGTCCACCCACAACAGTAGAAGGGAGAAATGAT
GAGAAGGCTCTTCTTGAACAGCTGGTATCCTTCCTTAGTGGCAAAGATGA
AACTGAGCTAGCTGAACTAGACAGAGCTCTGGGAATTGACAAACTTGTTC
AGGGGGGTGGATTAGATGTATTATCAGAGAGATTTCCACCACAACAAGCA
ACGCCACCTTTGATCATGGAAGAAAGACCCAACCTTTATTCCCAGCCTTA
CTCTTCTCCTTTTCCTACTGCCAATCTCCCTAGCCCTTTCCAAGGCATGG
TCAGGCAAAAACCTTCACTGGGGACGATGCCTGTTCAAGTAACACCTCCC
CGAGGTGCTTTTTCACCTGGCATGGGCATGCAGCCCAGGCAAACTCTAAA
CAGACCTCCGGCTGCACCTAACCAGCTTCGACTTCAACTACAGCAGCGAT
TACAGGGACAACAGCAGTTGATACACCAAAATCGGCAAGCTATCTTAAAC
CAGTTTGCAGCAACTGCTCCTGTTGGCATCAATATGAGATCAGGCATGCA
ACAGCAAATTACACCTCAGCCACCCCTGAATGCTCAAATGTTGGCACAAC
GTCAGCGGGAACTGTACAGTCAACAGCACCGACAGAGGCAGCTAATACAG
CAGCAAAGAGCCATGCTTATGAGGCAGCAAAGCTTTGGGAACAACCTCCC
TCCCTCATCTGGACTACCAGTTCAAACGGGGAACCCCCGTCTTCCTCAGG
GTGCTCCACAGCAATTCCCCTATCCACCAAACTATGGTACAAATCCAGGA
ACCCCACCTGCTTCTACCAGCCCGTTTTCACAACTAGCAGCAAATCCTGA
AGCATCCTTGGCCAACCGCAACAGCATGGTGAGCAGAGGCATGACAGGAA
ACATAGGAGGACAGTTTGGCACTGGAATCAATCCTCAGATGCAGCAGAAT
GTCTTCCAGTATCCAGGAGCAGGAATGGTTCCCCAAGGTGAGGCCAACTT
TGCTCCATCTCTAAGCCCTGGGAGCTCCATGGTGCCGATGCCAATCCCTC
CTCCTCAGAGTTCTCTGCTCCAGCAAACTCCACCTGCCTCCGGGTATCAG
TCACCAGACATGAAGGCCTGGCAGCAAGGAGCGATAGGAAACAACAATGT
GTTCAGTCAAGCTGTCCAGAACCAGCCCACGCCTGCACAGCCAGGAGTAT
ACAACAACATGAGCATCACCGTTTCCATGGCAGGTGGAAATACGAATGTT
CAGAACATGAACCCAATGATGGCCCAGATGCAGATGAGCTCTTTGCAGAT
GCCAGGAATGAACACTGTGTGCCCTGAGCAGATAAATGATCCCGCACTGA
GACACACAGGCCTCTACTGCAACCAGCTCTCATCCACTGACCTTCTCAAA
ACAGAAGCAGATGGAACCCAGCAGGTGCAACAGGTTCAGGTGTTTGCTGA
CGTCCAGTGTACAGTGAATCTGGTAGGCGGGGACCCTTACCTGAACCAGC
CTGGTCCACTGGGAACTCAAAAGCCCACGTCAGGACCACAGACCCCCCAG
GCCCAGCAGAAGAGCCTCCTTCAGCAGCTACTGACTGAATAACCACTTTT
AAAGGAATGTGAAATTTAAATAATAGACATACAGAGATATACAAATATAT
TATATATTTTTCTGAGATTTTTGATATCTCAATCTGCAGCCATTCTTCAG
GTCGTAGCATTTGGAGCAAAAAAAAAAAAAAAAAATCGATGTCGACTCGA
CGACACTACGGCGACAGCAACGG
PCIP

SEQ ID NO:127
>gi|5729725|ref|NM_006534.1|Homo sapiens nuclear
receptor coactivator 3 (NCOA3), mRNA
GTCGACGTGGCGGCCGGCGGCGGCTGCGGGCTGAGCGGCGAGTTTCCGAT
TTAAAGCTGAGCTGCGAGGAAAATGGCGGCGGGAGGATCAAAATACTTGC
TGGATGGTGGACTCAGAGACCAATAAAAATAAACTGCTTGAACATCCTTT
GACTGGTTAGCCAGTTGCTGATGTATATTCAAGATGAGTGGATTAGGAGA
AAACTTGGATCCACTGGCCAGTGATTCACGAAAACGCAAATTGCCATGTG
ATACTCCAGGACAAGGTCTTACCTGCAGTGGTGAAAAACGGAGACGGGAG
CAGGAAAGTAAATATATTGAAGAATTGGCTGAGCTGATATCTGCCAATCT
TAGTGATATTGACAATTTCAATGTCAAACCAGATAAATGTGCGATTTTAA
AGGAAACAGTAAGACAGATACGTCAAATAAAAGAGCAAGGAAAAACTATT
TCCAATGATGATGATGTTCAAAAAGCCGATGTATCTTCTACAGGGCAGGG
AGTTATTGATAAAGACTCCTTAGGACCGCTTTTACTTCAGGCATTGGATG
GTTTCCTATTTGTGGTGAATCGAGACGGAAACATTGTATTTGTATCAGAA
AATGTCACACAATACCTGCAATATAAGCAAGAGGACCTGGTTAACACAAG
TGTTTACAATATCTTACATGAAGAAGACAGAAAGGATTTTCTTAAGAATT
TACCAAAATCTACAGTTAATGGAGTTTCCTGGACAAATGAGACCCAAAGA
CAAAAAAGCCATACATTTAATTGCCGTATGTTGATGAAAACACCACATGA
TATTCTGGAAGACATAAACGCCAGTCCTGAAATGCGCCAGAGATATGAAA
CAATGCAGTGCTTTGCCCTGTCTCAGCCACGAGCTATGATGGAGGAAGGG
GAAGATTTGCAATCTTGTATGATCTGTGTGGCACGCCGCATTACTACAGG
AGAAAGAACATTTCCATCAAACCCTGAGAGCTTTATTACCAGACATGATC
TTTCAGGAAAGGTTGTCAATATAGATACAAATTCACTGAGATCCTCCATG
AGGCCTGGCTTTGAAGATATAATCCGAAGGTGTATTCAGAGATTTTTTAG
TCTAAATGATGGGCAGTCATGGTCCCAGAAACGTCACTATCAAGAAGTTA
CCAGTGATGGGATATTTTCCCCAACAGCTTATCTTAATGGCCATGCAGAA
ACCCCAGTATATCGATTCTCGTTGGCTGATGGAACTATAGTGACTGCACA
GACAAAAAGCAAACTCTTCCGAAATCCTGTAACAAATGATCGACATGGCT
TTGTCTCAACCCACTTCCTTCAGAGAGAACAGAATGGATATAGACCAAAC
CCAAATCCTGTTGGACAAGGGATTAGACCACCTATGGCTGGATGCAACAG
TTCGGTAGGCGGCATGAGTATGTCGCCAAACCAAGGCTTACAGATGCCGA
GCAGCAGGGCCTATGGCTTGGCAGACCCTAGCACCACAGGGCAGATGAGT
GGAGCTAGGTATGGGGGTTCCAGTAACATAGCTTCATTGACCCCTGGGCC
AGGCATGCAATCACCATCTTCCTACCAGAACAACAACTATAGGCTCAACA
TGAGTAGCCCCCCACATGGGAGTCCTGGTCTTGCCCCAAACCAGCAGAAT
ATCATGATTTCTCCTCGTAATCGTGGGAGTCCAAAGATAGCCTCACATCA
GTTTTCTCCTGTTGCAGGTGTGCACTCTCCCATGGCATCTTCTGGCAATA
CTGGGAACCACAGCTTTTCCAGCAGCTCTCTCAGTGCCCTGCAAGCCATC
AGTGAAGGTGTGGGGACTTCCCTTTTATCTACTCTGTCATCACCAGGCCC
CAAATTGGATAACTCTCCCAATATGAATATTACCCAACCAAGTAAAGTAA
GCAATCAGGATTCCAAGAGTCCTCTGGGCTTTTATTGCGACCAAAATCCA
GTGGAGAGTTCAATGTGTCAGTCAAATAGCAGAGATCACCTCAGTGACAA
AGAAAGTAAGGAGAGCAGTGTTGAGGGGGCAGAGAATCAAAGGGGTCCTT
TGGAAAGCAAAGGTCATAAAAAATTACTGCAGTTACTTACCTGTTCTTCT
GATGACCGGGGTCATTCCTCCTTGACCAACTCCCCCCTAGATTCAAGTTG
TAAAGAATCTTCTGTTAGTGTCACCAGCCCCTCTGGAGTCTCCTCCTCTA
CATCTGGAGGAGTATCCTCTACATCCAATATGCATGGGTCACTGTTACAA
GAGAAGCACCGGATTTTGCACAAGTTGCTGCAGAATGGGAATTCACCAGC
TGAGGTAGCCAAGATTACTGCAGAAGCCACTGGGAAAGACACCAGCAGTA
TAACTTCTTGTGGGGACGGAAATGTTGTCAAGCAGGAGCAGCTAAGTCCT
AAGAAGAAGGAGAATAATGCACTTCTTAGATACCTGCTGGACAGGGATGA
TCCTAGTGATGCACTCTCTAAAGAACTACAGCCCCAAGTGGAAGGAGTGG
ATAATAAAATGAGTCAGTGCACCAGCTCCACCATTCCTAGCTCAAGTCAA
GAGAAAGACCCTAAAATTAAGACAGAGACAAGTGAAGAGGGATCTGGAGA
CTTGGATAATCTAGATGCTATTCTTGGTGATCTGACTAGTTCTGACTTTT
ACAATAATTCCATATCCTCAAATGGTAGTCATCTGGGGACTAAGCAACAG
GTGTTTCAAGGAACTAATTCTCTGGGTTTGAAAAGTTCACAGTCTGTGCA
GTCTATTCGTCCTCCATATAACCGAGCAGTGTCTCTGGATAGCCCTGTTT
CTGTTGGCTCAAGTCCTCCAGTAAAAAATATCAGTGCTTTCCCCATGTTA
CCAAAGCAACCCATGTTGGGTGGGAATCCAAGAATGATGGATAGTCAGGA
AAATTATGGCTCAAGTATGGGAGACTGGGGCTTACCAAACTCAAAGGCCG
GCAGAATGGAACCTATGAATTCAAACTCCATGGGAAGACCAGGAGGAGAT
TATAATACTTCTTTACCCAGACCTGCACTGGGTGGCTCTATTCCCACATT
GCCTCTTCGGTCTAATAGCATACCAGGTGCGAGACCAGTATTGCAACAGC
AGCAGCAGATGCTTCAAATGAGGCCTGGTGAAATCCCCATGGGAATGGGG
GCTAATCCCTATGGCCAAGCAGCAGCATCTAACCAACTGGGTTCCTGGCC
CGATGGCATGTTGTCCATGGAACAAGTTTCTCATGGCACTCAAAATAGGC
CTCTTCTTAGGAATTCCCTGGATGATCTTGTTGGGCCACCTTCCAACCTG
GAAGGCCAGAGTGACGAAAGAGCATTATTGGACCAGCTGCACACTCTTCT
CAGCAACACAGATGCCACAGGCCTGGAAGAAATTGACAGAGCTTTGGGCA
TTCCTGAACTTGTCAATCAGGGACAGGCATTAGAGCCCAAACAGGATGCT
TTCCAAGGCCAAGAAGCAGCAGTAATGATGGATCAGAAGGCAGGATTATA
TGGACAGACATACCCAGCACAGGGGCCTCCAATGCAAGGAGGCTTTCATC
TTCAGGGACAATCACCATCTTTTAACTCTATGATGAATCAGATGAACCAG
CAAGGCAATTTTCCTCTCCAAGGAATGCACCCACGAGCCAACATCATGAG
ACCCCGGACAAACACCCCCAAGCAACTTAGAATGCAGCTTCAGCAGAGGC
TGCAGGGCCAGCAGTTTTTGAATCAGAGCCGACAGGCACTTGAATTGAAA
ATGGAAAACCCTACTGCTGGTGGTGCTGCGGTGATGAGGCCTATGATGCA
GCCCCAGCAGGGTTTTCTTAATGCTCAAATGGTCGCCCAACGCAGCAGAG
AGCTGCTAAGTCATCACTTCCGACAACAGAGGGTGGCTATGATGATGCAG
CAGCAGCAACAGCAGCAGCAGCAGCAGCAGCAGCAGCAACAGCAACAGCA
ACAGCAACAGCAGCAACAGCAGCAAACCCAGGCCTTCAGCCCACCTCCTA
ATGTGACTGCTTCCCCCAGCATGGATGGGCTTTTGGCAGGACCCACAATG
CCACAAGCTCCTCCGCAACAGTTTCCATATCAACCAAATTATGGAATGGG
ACAACAACCAGATCCAGCCTTTGGTCGAGTGTCTAGTCCTCCCAATGCAA
TGATGTCGTCAAGAATGGGTCCCTCCCAGAATCCCATGATGCAACACCCG
CAGGCTGCATCCATCTATCAGTCCTCAGAAATGAAGGGCTGGCCATCAGG
AAATTTGGCCAGGAACAGCTCCTTTTCCCAGCAGCAGTTTGCCCACCAGG
GGAATCCTGCAGTGTATAGTATGGTGCACATGAATGGCAGCAGTGGTCAC
ATGGGACAGATGAACATGAACCCCATGCCCATGTCTGGCATGCCTATGGG
TCCTGATCAGAAATACTGCTGACATCTCTGCACCAGGACCTCTTAAGGAA
ACCACTGTACAAATGACACTGCACTAGGATTATTGGGAAGGAATCATTGT
TCCAGGCATCCATCTTGGAAGAAAGGACCAGCTTTGAGCTCCATCAAGGG
TATTTTAAGTGATGTCATTTGAGCAGGACTGGATTTTAAGCCGAAGGGCA
ATATCTACGTGTTTTTCCCCCCTCCTTCTGCTGTGTATCATGGTGTTCAA
AACAGAAATGTTTTTTGGCATTCCACCTCCTAGGGATATAATTCTGGAGA
CATGGAGTGTTACTGATCATAAAACTTTTGTGTCACTTTTTTCTGCCTTG
CTAGCCAAAATCTCTTAAATACACGTAGGTGGGCCAGAGAACATTGGAAG
AATCAAGAGAGATTAGAATATCTGGTTTCTCTAGTTGCAGTATTGGACAA
AGAGCATAGTCCCAGCCTTCAGGTGTAGTAGTTCTGTGTTGACCCTTTGT
CCAGTGGAATTGGTGATTCTGAATTGTCCTTTACTAATGGTGTTGAGTTG
CTCTGTCCCTATTATTTGCCCTAGGCTTTCTCCTAATGAAGGTTTTCATT
TGCCATTCATGTCCTGTAATACTTCACCTCCAGGAACTGTCATGGATGTC
CAAATGGCTTTGCAGAAAGGAAATGAGATGACAGTATTTAATCGCAGCAG
TAGCAAACTTTTCACATGCTAATGTGCAGCTGAGTGCACTTTATTTAAAA
AGAATGGATAAATGCAATATTCTTGAGGTCTTGAGGGAATAGTGAAACAC
ATTCCTGGTTTTTGCCTACACTTACGTGTTAGACAAGAACTATGATTTTT
TTTTTTAAAGTACTGGTGTCACCCTTTGCCTATATGGTAGAGCAATAATG
CTTTTTAAAAATAAACTTCTGAAAACCCAAGGCCAGGTACTGCATTCTGA
ATCAGAATCTCGCAGTGTTTCTGTGAATAGATTTTTTTGTAAATATGACC
TTTAAGATATTGTATTATGTAAAATATGTATATACCTTTTTTTGTAGGTC
ACAACAACTCATTTTTACAGAGTTTGTGAAGCTAAATATTTAACATTGTT
GATTTCAGTAAGCTGTGTGGTGAGGCTACCAGTGGAAGAGACATCCCTTG
ACTTTTGTGGCCTGGGGGAGGGGTAGTGCACCACAGCTTTTCCTTCCCCA
CCCCCCAGCCTTAGATGCCTCGCTCTTTTCAATCTCTTAATCTAAATGCT
TTTTAAAGAGATTATTTGTTTAGATGTAGGCATTTTAATTTTTTAAAAAT
TCCTCTACCAGAACTAAGCACTTTGTTAATTTGGGGGGAAAGAATAGATA
TGGGGAAATAAACTTAAAAAAAAATCAGGAATTTAAAAAAAACGAGCAAT
TTGAAGAGAATCTTTTGGATTTTAAGCAGTCCGAAATAATAGCAATTCAT
GGGCTGTGTGTGTGTGTGTATGTGTGTGTGTGTGTGTGTATGTTTAATTA
TGTTACCTTTTCATCCCCTTTAGGAGCGTTTTCAGATTTTGGTTCGTAAG
ACCTGAATCCCATATTGAGATCTCGAGTAGAATCCTTGGTGTGGTTTCTG
GTGTCTGCTCAGCTGTCCCCTCATTCTACTAATGTGATGCTTTCATTATG
TCCCTGTGGATTAGAATAGTGTCAGTTATTTCTTAAGTAACTCAGTACCC
AGAACAGCCAGTTTTACTGTGATTCAGAGCCACAGTCTAACTGAGCACCT
TTTAAACCCCTCCCTCTTCTGCCCCCTACCACTTTTCTGCTGTTGCCTCT
CTTTGACACCTGTTTTAGTCAGTTGGGAGGAAGGGAAAAATCAAGTTTAA
TTCCCTTTATCTGGGTTAATTCATTTGGTTCAAATAGTTGACGGAATTGG
GTTTCTGAATGTCTGTGAATTTCAGAGGTCTCTGCTAGCCTTGGTATCAT
TTTCTAGCAATAACTGAGAGCCAGTTAATTTTAAGAATTTCACACATTTA
GCCAATCTTTCTAGATGTCTCTGAAGGTAAGATCATTTAATATCTTTGAT
ATGCTTACGAGTAAGTGAATCCTGATTATTTCCAGACCCACCACCAGAGT
GGATCTTATTTTCAAAGCAGTATAGACAATTATGAGTTTGCCCTCTTTCC
CCTACCAAGTTCAAAATATATCTAAGAAAGATTGTAAATCCGAAAACTTC
CATTGTAGTGGCCTGTGCTTTTCAGATAGTATACTCTCCTGTTTGGAGAC
AGAGGAAGAACCAGGTCAGTCTGTCTCTTTTTCAGCTCAATTGTATCTGA
CCCTTCTTTAAGTTATGTGTGTGGGGAGAAATAGAATGGTGCTCTTATGT
CGAC
GRIP1
SEQ ID NO:127

gi|5729857|ref|NM_006540.1|Homo sapiens nuclear
receptor coactivator 2 (NCOA2), mRNA
GGCGGCCGCAGCCTCGGCTACAGCTTCGGCGGCGAAGGTCAGCGCCGACG
GCAGCCGGCACCTGACGGCGTGACCGACCCGAGCCGATTTCTCTTGGATT
TGGCTACACACTTATAGATCTTCTGCACTGTTTACAGGCACAGTTGCTGA
TATGTGTTCAAGATGAGTGGGATGGGAGAAAATACCTCTGACCCCTCCAG
GGCAGAGACAAGAAAGCGCAAGGAATGTCCTGACCAACTTGGACCCAGCC
CCAAAAGGAACACTGAAAAACGTAATCGTGAACAGGAAAATAAATATATA
GAAGAACTTGCAGAGTTGATTTTTGCAAATTTTAATGATATAGACAACTT
TAACTTCAAACCTGACAAATGTGCAATCTTAAAAGAAACTGTGAAGCAAA
TTCGTCAGATCAAAGAACAAGAGAAAGCAGCAGCTGCCAACATAGATGAA
GTGCAGAAGTCAGATGTATCCTCTACAGGGCAGGGTGTCATCGACAAGGA
TGCGCTGGGGCCTATGATGCTTGAGGCCCTTGATGGGTTCTTCTTTGTAG
TGAACCTGGAAGGCAACGTTGTGTTTGTGTCAGAGAATGTGACACAGTAT
CTAAGGTATAACCAAGAAGAGCTGATGAACAAAAGTGTATATAGCATCTT
GCATGTTGGGGACCACACGGAATTTGTCAAAAACCTGCTGCCAAAGTCTA
TAGTAAATGGGGGATCTTGGTCTGGCGAACCTCCGAGGCGGAACAGCCAT
ACCTTCAATTGTCGGATGCTGGTAAAACCTTTACCTGATTCAGAAGAGGA
GGGTCATGATAACCAGGAAGCTCATCAGAAATATGAAACTATGCAGTGCT
TCGCTGTCTCTCAACCAAAGTCCATCAAAGAAGAAGGAGAAGATTTGCAG
TCCTGCTTGATTTGCGTGGCAAGAAGAGTTCCCATGAAGGAAAGACCAGT
TCTTCCCTCATCAGAAAGTTTTACTACTCGCCAGGATCTCCAAGGCAAGA
TCACGTCTCTGGATACCAGCACCATGAGAGCAGCCATGAAACCAGGCTGG
GAGGACCTGGTAAGAAGGTGTATTCAGAAGTTCCATGCGCAGCATGAAGG
AGAATCTGTGTCCTATGCTAAGAGGCATCATCATGAAGTACTGAGACAAG
GATTGGCATTCAGTCAAATCTATCGTTTTTCCTTGTCTGATGGCACTCTT
GTTGCTGCACAAACGAAGAGCAAACTCATCCGTTCTCAGACTACTAATGA
ACCTCAACTTGTAATATCTTTACATATGCTTCACAGAGAGCAGAATGTGT
GTGTGATGAATCCGGATCTGACTGGACAAACGATGGGGAAGCCACTGAAT
CCAATTAGCTCTAACAGCCCTGCCCATCAGGCCCTGTGCAGTGGGAACCC
AGGTCAGGACATGACCCTCAGTAGCAATATAAATTTTCCCATAAATGGCC
CAAAGGAACAAATGGGCATGCCCATGGGCAGGTTTGGTGGTTCTGGGGGA
ATGAACCATGTGTCAGGCATGCAAGCAACCACTCCTCAGGGTAGTAACTA
TGCACTCAAAATGAACAGCCCCTCACAAAGCAGCCCTGGCATGAATCCAG
GACAGCCCACCTCCATGCTTTCACCAAGGCATCGCATGAGCCCTGGAGTG
GCTGGCAGCCCTCGAATCCCACCCAGTCAGTTTTCCCCTGCAGGAAGCTT
GCATTCCCCTGTGGGAGTTTGCAGCAGCACAGGAAATAGCCATAGTTATA
CCAACAGCTCCCTCAATGCACTTCAGGCCCTCAGCGAGGGGCACGGGGTC
TCATTAGGGTCATCGTTGGCTTCACCAGACCTAAAAATGGGCAATTTGCA
AAACTCCCCAGTTAATATGAATCCTCCCCCACTCAGCAAGATGGGAAGCT
TGGACTCAAAAGACTGTTTTGGACTATATGGGGAGCCCTCTGAAGGTACA
ACTGGACAAGCAGAGAGCAGCTGCCATCCTGGAGAGCAAAAGGAAACAAA
TGACCCCAACCTGCCCCCGGCCGTGAGCAGTGAGAGAGCTGACGGGCAGA
GCAGACTGCATGACAGCAAAGGGCAGACCAAACTCCTGCAGCTGCTGACC
ACCAAATCTGATCAGATGGAGCCCTCGCCCTTAGCCAGCTCTTTGTCGGA
TACAAACAAAGACTCCACAGGTAGCTTGCCTGGTTCTGGGTCTACACATG
GAACCTCGCTCAAGGAGAAGCATAAAATTTTGCACAGACTCTTGCAGGAC
AGCAGTTCCCCTGTGGACTTGGCCAAGTTAACAGCAGAAGCCACAGGCAA
AGACCTGAGCCAGGAGTCCAGCAGCACAGCTCCTGGATCAGAAGTGACTA
TTAAACAAGAGCCGGTGAGCCCCAAGAAGAAAGAGAATGCACTACTTCGC
TATTTGCTAGATAAAGATGATACTAAAGATATTGGTTTACCAGAAATAAC
CCCCAAACTTGAGAGACTGGACAGTAAGACAGATCCTGCCAGTAACACAA
AATTAATAGCAATGAAAACTGAGAAGGAGGAGATGAGCTTTGAGCCTGGT
GACCAGCCTGGCAGTGAGCTGGACAACTTGGAGGAGATTTTGGATGATTT
GCAGAATAGTCAATTACCACAGCTTTTCCCAGACACGAGGCCAGGCGCCC
CTGCTGGATCAGTTGACAAGCAAGCCATCATCAATGACCTCATGCAACTC
ACAGCTGAAAACAGCCCTGTCACACCTGTTGGAGCCCAGAAAACAGCACT
GCGAATTTCACAGAGCACTTTTAATAACCCACGACCAGGGCAACTGGGCA
GGTTATTGCCAAACCAGAATTTACCACTTGACATCACATTGCAAAGCCCA
ACTGGTGCTGGACCTTTCCCACCAATCAGAAACAGTAGTCCCTACTCAGT
GATACCTCAGCCAGGAATGATGGGTAATCAAGGGATGATAGGAAACCAAG
GAAATTTAGGGAACAGTAGCACAGGAATGATTGGTAACAGTGCTTCTCGG
CCTACTATGCCATCTGGAGAATGGGCACCGCAGAGTTCGGCTGTGAGAGT
CACCTGTGCTGCTACCACCAGTGCCATGAACCGGCCAGTCCAAGGAGGTA
TGATTCGGAACCCAGCAGCCAGCATCCCCATGAGGCCCAGCAGCCAGCCT
GGCCAAAGACAGACGCTTCAGTCTCAGGTCATGAATATAGGGCCATCTGA
ATTAGAGATGAACATGGGGGGACCTCAGTATAGCCAACAACAAGCTCCTC
CAAATCAGACTGCCCCATGGCCTGAAAGCATCCTGCCTATAGACCAGGCG
TCTTTTGCCAGCCAAAACAGGCAGCCATTTGGCAGTTCTCCAGATGACTT
GCTATGTCCACATCCTGCAGCTGAGTCTCCGAGTGATGAGGGAGCTCTCC
TGGACCAGCTGTATCTGGCCTTGCGGAATTTTGATGGCCTGGAGGAGATT
GATAGAGCCTTAGGAATACCCGAACTGGTCAGCCAGAGCCAAGCAGTAGA
TCCAGAACAGTTCTCAAGTCAGGATTCCAACATCATGCTGGAGCAGAAGG
CGCCCGTTTTCCCACAGCAGTATGCATCTCAGGCACAAATGGCCCAGGGT
AGCTATTCTCCCATGCAAGATCCAAACTTTCACACCATGGGACAGCGGCC
TAGTTATGCCACACTCCGTATGCAGCCCAGACCGGGCCTCAGGCCCACGG
GCCTAGTGCAGAACCAGCCAAATCAACTAAGACTTCAACTTCAGCATCGC
CTCCAAGCACAGCAGAATCGCCAGCCACTTATGAATCAAATCAGCAATGT
TTCCAATGTGAACTTGACTCTGAGGCCTGGAGTACCAACACAGGCACCTA
TTAATGCACAGATGCTGGCCCAGAGACAGAGGGAAATCCTGAACCAGCAT
CTTCGACAGAGACAAATGCATCAGCAACAGCAAGTTCAGCAACGAACTTT
GATGATGAGAGGACAAGGGTTGAATATGACACCAAGCATGGTGGCTCCTA
GTGGTATGCCAGCAACTATGAGCAACCCTCGGATTCCCCAGGCAAATGCA
CAGCAGTTTCCATTTCCTCCAAACTACGGAATAAGTCAGCAACCTGATCC
AGGCTTTACTGGGGCTACGACTCCCCAGAGCCCACTTATGTCACCCCGAA
TGGCACATACACAGAGTCCCATGATGCAACAGTCTCAGGCCAACCCAGCC
TATCAGGCCCCCTCCGACATAAATGGATGGGCGCAGGGGAACATGGGCGG
AAACAGCATGTTTTCCCAGCAGTCCCCACCACACTTTGGGCAGCAAGCAA
ACACCAGCATGTACAGTAACAACATGAACATCAATGTGTCCATGGCGACC
AACACAGGTGGCATGAGCAGCATGAACCAGATGACAGGACAGATCAGCAT
GACCTCAGTGACCTCCGTGCCTACGTCAGGGCTGTCCTCCATGGGTCCCG
AGCAGGTTAATGATCCTGCTCTGAGGGGAGGCAACCTGTTCCCAAACCAG
CTGCCTGGAATGGATATGATTAAGCAGGAGGGAGACACAACACGGAAATA
TTGCTGACACTGCTGAAGCCAGTTGCTTCTTCAGCTGACCGGGCTCACTT
GCTCAAAACACTTCCAGTCTGGAGAGCTGTGTCTATTTGTTTCAACCCAA
CTGACCTGCCAGCCGGTTCTGCTAGAGCAGACAGGCCTGGCCCTGGTTCC
CAGGGTGGCGTCCACTCGGCTGTGGCAGGAGGAGCTGCCTGTTCTCTTGA
CAGTCTGAAGCTCGCATCCAGACAGTCGCTCAGTCTGTTCCCTGCATTCA
CCTTAGTGCAACTTAGATCTCTCCTCCCCAAGTAAATGTTGACAGGCCAA
TTTCATACCCATGTCAGATTGAATGTATTTAAATGTATGTATTTAAGGAG
AACCATGCTCTTGTTCTGTTCCTGTTCGGTTCCAGACACTGGTTTCTTGC
TTTGTTTTCCCTGGCTAACAGTCTAGTGCCAAAGATTAAGATTTTATCTG
GGGGAAAGAAAAGAATTTTTTAAAAAATTAAACTAAAGATGTTTTAAGCT
AAAGCCTGAATTTGGGATGGAAGCAGGACAGACACCGTGGACAGCGCTGT
ATTTACAGACACACCCAGTGCGTGAAGACCAACAAAGTCACAGTCGTATC
TCTAGAAAGCTCTAAAGACCATGTTGGAAAGAGTCTCCAGTTACTGAACA
GATGAAAAGGAGCCTGTGAGAGGGCTGTTAACATTAGCAAATATTTTTTC
CTTGTTTTTTCTTTGTTAAAACCAAACTGGTTCACCTGAATCATGAATTG
AGAAGAAATAATTTTCATTTCTAAATTAAGTCCCTTTTAGTTTGATCAGA
CAGCTTGAATCAGCATCTCTTCTTCCCTGTCAGCCTGACTCTTCCCTTCC
CCTCTCTCATTCCCCATACTCCCTATTTTCATTCCTTTTTTAAAAAATAA
TATAAGCTACAGAAACCAGGTAAGCCCTTTATTTCCTTAAATGTTTTGCC
AGCCACTTACCAATTGCTAAGTATTGAATTTCAGAAAAAAAAAATGCATT
TACTGGCAAGGAGAAGAGCAAAGTTAAGGCTTGATACCAATCGAGCTAAG
GATACCTGCTTTGGAAGCATGTTTATTCTGTTCCCCAGCAACTCTGGCCT
CCAAAATGGGAGAAACGCCAGTGTGTTTAAATTGATAGCAGATATCACGA
CAGATTTAACCTCTGCCATGTGTTTTTTATTTTGTTTTTTAGCAGTGCTG
ACTAAGCCGAAGTTTTGTAAGGTACATAAAATCCAATTTATATGTAAACA
AGCAATAATTTAAGTTGAGAACTTATGTGTTTTAATTGTATAATTTTTGT
GAGGTATACATATTGTGGAATTGACTCAAAAATGAGGTACTTCAGTATTA
AATTAGATATCTTCATAGCAATGTCTCCTAAAGGTGTTTTGTAAAGGATA
TCAATGCCTTGATTAGACCTAATTTGTAGACTTAAGACTTTTTATTTTCT
AAACCTTGTGATTCTGCTTATAAGTCATTTATCTAATCTATATGATATGC
AGCCGCTGTAGGAACCAATTCTTGATTTTTATATGTTTATATTCTTTCTT
AATGAACCTTAGAAAGACTACATGTTACTAAGCAGGCCACTTTTATGGTT
GTTTTT
ATF-2

SEQ ID NO:128

>gi|4503032|ref|NM_001880.1|Homo sapiens acti-
vating transcription factor 2 (ATF2), mRNA
GAATTCTGTGATAAGTTATTCAACTTATGAAATTCAAGTTACATGTGAAT
TCTGCCAGGCAATACAAGGACCTGTGGAATATGAGTGATGACAAACCCTT
TCTATGTACTGCGCCTGGATGTGGCCAGCGTTTTACCAACGAGGATCATT
TGGCTGTCCATAAACATAAACATGAGATGACACTGAAATTTGGTCCAGCA
CGTAATGACAGTGTCATTGTGGCTGATCAGACCCCAACACCAACAAGATT
CTTGAAAAACTGTGAAGAAGTGGGTTTGTTTAATGAGTTGGCGAGTCCAT
TTGAGAATGAATTCAAGAAAGCTTCAGAAGATGACATTAAAAAAATGCCT
CTAGATTTATCCCCTCTTGCAACACCTATCATAAGAAGCAAAATTGAGGA
GCCTTCTGTTGTAGAAACAACTCACCAGGATAGTCCTTTACCTCACCCAG
AGTCTACTACCAGTGATGAGAAGGAAGTACCATTGGCACAAACTGCACAG
CCCACATCAGCTATTGTTCGTCCAGCATCATTACAGGTTCCCAATGTGCT
GCTTACAAGTTCTGACTCAAGTGTAATTATTCAGCAGGCAGTACCTTCAC
CAACCTCAAGTACTGTAATCACCCAGGCACCATCCTCTAACAGGCCAATT
GTCCCTGTACCAGGCCCATTTCCTCTTCTGTTACATCTTCCTAGTGGACA
AACCATGCCTGTTGCTATTCCTGCATCAATTACAAGTTCTAATGTGCATG
TTCCAGCTGCAGTCCCACTCGTTCGACCAGTCACCATGGTGCCTAGTGTT
CCAGGAATCCCAGGTCCTTCCTCTCCCCAACCAGTACAGTCAGAAGCAAA
AATGAGATTAAAAGCTGCTTTGACCCAGCAACATCCTCCAGTTACCAATG
GTGATACTGTCAAAGGTCATGGTAGCGGATTGGTTAGGACTCAGTCAGAG
GAATCTCGACCGCAGTCATTACAACAGCCAGCCACATCCACTACAGAAAC
TCCGGCTTCTCCAGCTCACACAACTCCACAGACCCAAAGTACAAGTGGTC
GTCGGAGAAGAGCAGCTAACGAAGATCCTGATGAAAAAAGGAGAAAGTTT
TTAGAGCGAAATAGAGCAGCAGCTTCAAGATGCCGACAAAAAAGGAAAGT
CTGGGTTCAGTCTTTAGAGAAGAAAGCTGAAGACTTGAGTTCATTAAATG
GTCAGCTGCAGAGTGAAGTCACCCTGCTGAGAAATGAAGTGGCACAGCTG
AAACAGCTTCTTCTGGCTCATAAAGATTGCCCTGTAACCGCCATGCAGAA
GAAATCTGGCTATCATACTGCTGATAAAGATGATAGTTCAGAAGACATTT
CAGTGCCGAGTAGTCCACATACGGAAGCTATACAGCATAGTTCGGTCAGC
ACATCCAATGGAGTCAGTTCAACCTCCAAGGCAGAAGCTGTAGCCACTTC
AGTCCTCACCCAGATGGCGGACCAGAGTACAGAGCCTGCTCTTTCACAGA
TCGTTATGGCTCCTTCCTCCCAGTCACAGCCCTCAGGAAGTTGATTAAAA
ACCTGCAGTACAACAGTTTAGATACTCATTAGTGACTTCAAAGGGAAATC
AAGGAAAGACCAGTTTCCATTTATGCGAAATCTGTGGTTGTAAATTT

GNAT related

HAT1

SEQ ID NO:129

>gi|4504340|ref|NM_003642.1|Homo sapiens histone
acetyltransferase 1 (HAT1), mRNA
CGTCCTTCCTCAGCCGCGGGTGATCGTAGCTCGGAAATGGCGGGATTTGG
TGCTATGGAGAAATTTTTGGTAGAATATAAGAGTGCAGTGGAGAAGAAAC
TGGCAGAGTACAAATGTAACACCAACACAGCAATTGAACTAAAATTAGTT
CGTTTTCCTGAAGATCTTGAAAATGACATTAGAACTTTCTTTCCTGAGTA
TACCCATCAACTCTTTGGGGATGATGAAACTGCTTTTGGTTACAAGGGTC
TAAAGATCCTGTTATACTATATTGCTGGTAGCCTGTCAACAATGTTCCGT
GTTGAATATGCATCTAAAGTTGATGAGAACTTTGACTGTGTAGAGGCAGA
TGATGTTGAGGGCAAAATTAGACAAATCATTCCACCTGGATTTTGCACAA
ACACGAATGATTTCCTTTCTTTACTGGAAAAGGAAGTTGATTTCAAGCCA
TTCGGAACCTTACTTCATACCTACTCAGTTCTCAGTCCAACAGGAGGAGA
AAACTTTACCTTTCAGATATATAAGGCTGACATGACATGTAGAGGCTTTC
GAGAATATCATGAAAGGCTTCAGACCTTTTTGATGTGGTTTATTGAAACT
GCTAGCTTTATTGACGTGGATGATGAAAGATGGCACTACTTTCTAGTATT
TGAGAAGTATAATAAGGATGGAGCTACGCTCTTTGCGACCGTAGGCTACA
TGACAGTCTATAATTACTATGTGTACCCAGACAAAACCCGGCCACGTGTA
AGTCAGATGCTGATTTTGACTCCATTTCAAGGTCAAGGCCATGGTGCTCA
ACTTCTTGAAACAGTTCATAGATACTACACTGAATTTCCTACAGTTCTTG
ATATTACAGCGGAAGATCCATCCAAAAGCTATGTGAAATTACGAGACTTT
GTGCTTGTGAAGCTTTGTCAAGATTTGCCCTGTTTTTCCCGGGAAAAATT
AATGCAAGGATTCAATGAAGATATGGCGATAGAGGCACAACAGAAGTTCA
AAATAAATAAGCAACACGCTAGAAGGGTTTATGAAATTCTTCGACTACTG
GTAACTGACATGAGTGATGCCGAACAATACAGAAGCTACAGACTGGATAT
TAAAAGAAGACTAATTAGCCCATATAAGAAAAAGCAGAGAGATCTTGCTA
AGATGAGAAAATGTCTCAGACCAGAAGAACTGACAAACCAGATGAACCAA
ATAGAAATAAGCATGCAACATGAACAGCTGGAAGAGAGTTTTCAGGAACT
AGTGGAAGATTACCGGCGTGTTATTGAACGACTTGCTCAAGAGTAAAGAT
TATACTGCTCTGTACAGGAAGCTTGCAAATTTTCTGTACAATGTGCTGTG
AAAAATCTGATGACTTTAATTTTAAAATCTTGTGACATTTTGCTTATACT
AAAAGTTATCTATCTTTAGTTGAATATTTTCTTTTGGAGAGATTGTATAT
TTTAAAATACTGTTTAGAGTTTATGAGCATATATTGCATTTAAAGAAAGA
TAAAGCTTCTGAAATACTACTGCAATTGCTTCCCTTCTTAAACAGTATAA
TAAATGCTTAGTTGTGAT

TABLE 6
ATP-dependent Chromatin Remodelling.

SMARCA5

SEQ ID NO:130

>gi|4507074|ref|NM_003601.1|Homo sapiens SWI/SNF
related, matrix associated, actin dependent
regulator of chromatin, subfamily a, member 5
(SMARCA5), mRNA
GCGGAAGAGCAGAACGTTTGGGAGTGTGCAGCTCCTGGGCCCGGCTCAGG
CCCGTCGCGGAGGCGCGGCGCAGGGGAGCGCTCGGGTGGGAGTCTCGCTC
CTCCACCAGTTTATTGCGACGTAGCATCCAGGCCTAGGCCTCCCCGTCCA
TCCCCGCCGGACTCGGGCCTCTGGCAGCAGCGGGTGACGCAGACGGAACA
TCATGTCGTCCGCGGCCGAGCCTCCGCCACCCCCGCCTCCCGAGAGCGCG
CCTTCCAAGCCCGCAGCCTCGATCGCCAGCGGCGGGAGCAACAGCAGCAA
CAAAGGCGGCCCCGAAGGCGTCGCGGCGCAGGCGGTTGCGTCTGCGGCCA
GCGCTGGTCCCGCAGACGCCGAGATGGAGGAAATATTTGATGATGCGTCA
CCTGGAAAGCAAAAGGAAATCCAAGAACCAGATCCTACCTATGAAGAAAA
AATGCAAACTGACCGGGCAAATAGATTCGAGTATTTATTAAAGCAGACAG
AACTTTTTGCACATTTCATTCAACCTGCTGCTCAGAAGACTCCAACTTCA
CCTTTGAAGATGAAACCAGGGCGCCCACGAATAAAAAAAGATGAGAAGCA
GAACTTACTATCCGTTGGCGATTACCGACACCGTAGAACAGAGCAAGAGG
AGGATGAAGAGCTATTAACAGAAAGCTCCAAAGCAACCAATGTTTGCACT
CGATTTGAAGACTCTCCATCGTATGTAAAATGGGGTAAACTGAGAGATTA
TCAGGTCCGAGGATTAAACTGGCTCATTTCTTTGTATGAGAATGGCATCA
ATGGTATCCTTGCAGATGAAATGGGCCTAGGAAAGACTCTTCAAACAATT
TCTCTTCTTGGGTACATGAAACATTATAGAAACATTCCTGGGCCTCATAT
GGTTTTGGTTCCTAAGTCTACATTACACAACTGGATGAGTGAATTCAAGA
GATGGGTACCAACACTTAGATCTGTTTGTTTGATAGGAGATAAAGAACAA
AGAGCTGCTTTTGTCAGAGACGTTTTATTACCGGGAGAATGGGATGTATG
TGTAACATCTTATGAAATGCTTATTAAAGAGAAGTCTGTGTTCAAAAAAT
TTAATTGGAGATACTTAGTAATAGATGAAGCTCACAGGATCAAAAATGAA
AAATCTAAGTTGTCAGAAATAGTGAGGGAATTCAAGACTACAAATAGACT
ATTATTAACTGGAACACCTCTTCAGAACAACTTGCATGAGCTGTGGTCAC
TTCTTAACTTTCTGTTGCCAGATGTGTTTAATTCAGCAGATGACTTTGAT
TCCTGGTTTGATACAAACAACTGCCTTGGGGATCAAAAACTAGTTGAGAG
GCTTCATATGGTTTTGCGTCCATTCCTCCTTCGTCGAATTAAGGCTGATG
TTGAAAAGAGTTTGCCTCCAAAGAAGGAAGTAAAAATCTATGTGGGCCTC
AGCAAAATGCAAAGGGAATGGTATACTCGGATATTAATGAAGGATATAGA
TATACTCAACTCAGCAGGCAAGATGGACAAAATGAGGTTATTGAACATCC
TAATGCAGTTGAGAAAATGTTGTAATCATCCATATGTCTTTGATGGAGCA
GAACCTGGTCCACCTTATACAACAGATATGCATCTAGTAACCAACAGTGG
CAAAATGGTGGTTTTAGACAAGCTGCTCCCTAAGTTAAAAGAACAAGGTT
CACGAGTACTAATCTTCAGTCAAATGACAAGGGTATTGGACATTTTGGAA
GATTATTGCATGTGGAGAAATTATGAGTACTGCAGGTTGGATGGTCAGAC
ACCCCATGATGAGAGACAAGACTCCATCAATGCATACAATGAACCAAACA
GCACAAAGTTTGTTTTCATGTTAAGCACGCGTGCTGGTGGTCTTGGCATC
AATCTTGCGACTGCTGATGTAGTAATTTTGTATGATTCTGATTGGAATCC
CCAAGTAGATCTTCAGGCTATGGACCGAGCACATAGAATTGGGCAGACTA
AGACAGTCAGAGTGTTCCGCTTTATAACTGATAACACTGTAGAAGAAAGA
ATAGTAGAACGTGCTGAGATGAAACTCAGACTGGATTCAATAGTCATTCA
ACAAGGGAGGCTTGTGGATCAGAATCTGAACAAAATTGGGAAAGATGAAA
TGCTTCAAATGATTAGACATGGAGCAACACATGTGTTTGCTTCAAAGGAA
AGTGAGATCACTGATGAAGATATCGATGGTATTTTGGAAAGAGGTGCAAA
GAAGACTGCAGAGATGAATGAAAAGCTCTCCAAGATGGGCGAAAGTTCAC
TTAGAAACTTTACAATGGATACAGAGTCAAGTGTTTATAACTTCGAAGGA
GAAGACTATAGAGAAAAACAAAAGATTGCATTCACAGAGTGGATTGAACC
ACCTAAACGAGAAAGAAAAGCCAACTATGCCGTTGATGCATATTTCAGGG
AAGCTCTTCGTGTTAGTGAACCTAAAGCACCCAAGGCTCCTCGACCTCCA
AAACAACCCAATGTTCAGGATTTCCAGTTCTTTCCTCCACGTTTATTTGA
ATTACTGGAAAAAGAAATTCTGTTTTACAGAAAAACTATTGGGTACAAGG
TACCTCGAAATCCTGAGCTGCCTAACGCAGCACAGGCACAAAAAGAAGAA
CAGCTTAAAATTGATGAAGCTGAATCCCTTAATGATGAAGAGTTAGAGGA
AAAAGAGAAGCTTCTAACACAGGGATTTACCAATTGGAATAAGAGAGATT
TTAACCAGTTTATCAAAGCTAATGAGAAGTGGGGTCGTGATGATATTGAA
AATATAGCAAGAGAAGTAGAAGGCAAAACTCCAGAAGAAGTCATTGAATA
TTCAGCTGTGTTTTGGGAAAGGTGCAACGAGCTCCAGGACATAGAGAAGA
TTATGGCTCAGATTGAAAGGGGAGAGGCGAGAATTCAAAGAAGAATAAGC
ATCAAGAAAGCACTTGACACAAAGATTGGACGGTACAAAGCACCTTTTCA
TCAGCTGAGAATATCATATGGTACTAACAAAGGAAAAAACTATACTGAAG
AAGAAGATCGTTTTCTGATTTGTATGCTTCACAAACTTGGATTTGACAAA
GAAAATGTTTATGATGAATTGCGACAGTGTATTCGCAACTCTCCTCAGTT
CAGATTTGACTGGTTTCTTAAGTCCAGAACTGCAATGGAGCTCCAGAGGA
GATGTAATACCTTAATTACTTTGATTGAAAGAGAAAACATGGAACTAGAA
GAAAAGGAGAAGGCAGAGAAAAAGAAACGAGGACCAAAGCCTTCAACACA
GAAACGTAAAATGGATGGCGCACCTGATGGTCGAGGAAGAAAAAAGAAGC
TGAAACTATGAATATGTTTTTGTTTCATAATCACTAACTTTAAACCAGTA
GTTCTTTAATTTACGGGTCTTCATAAGATGTACTGTACAATGCTCAATTG
TTATGTCATTTAAAGACATCAGGTTCATCTGTTTACTGAGCTAGAAACAT
AGTATGTAGTTTCACTTTTTTAAATGCAACAGCTGTGCTGAAATTTTTTT
ATCATTAACACTTGAAGTAATAAAATAGGCTTCATTTATTAAAAAAAAAA
AAAAAA
SMARCA2

SEQ ID NO:131

gi|4507068|ref|NM-003070.1|Homo sapiens SWI/SNF
related, matrix associated, actin dependent
regulator of chromatin, subfamily a, member 2
(SMARCA2), mRNA
AAAAATTTTCTGTTACCAAATTTTACAACTTCTAATAAGACTACTATAAC
TTTATGTAAACTGATGAAGATGTGCTGATTAACATATTCTGTGATATGGT
TTACAACTTTTAATCATAATTGTCCATGATTTTGGAATGCTGTTATTTAT
CAGTAAATGTAAAATATTTGAGGCATTTAGCCATACACACACTAGAACTT
TTTAAAACTTTGTCCTATAGTGTAATTATAAACTGATGACTATTATCTTC
ATACATTGAGTCTTCATGCATCAATGAAATGAAAAATATAGGAGTAGATG
TCCACGCCCACAGACCCTGGTGCGATGCCCCACCCAGGGCCTTCGCCGGG
GCCTGGGCCTTCCCCTGGGCCAATTCTTGGGCCTAGTCCAGGACCAGGAC
CATCCCCAGGTTCCGTCCACAGCATGATGGGGCCAAGTCCTGGACCTCCA
AGTGTCTCCCATCCTATGCCGACGATGGGGTCCACAGACTTCCCACAGGA
AGGCATGCATCAAATGCATAAGCCCATCGATGGTATACATGACAAGGGGA
TTGTAGAAGACATCCATTGTGGATCCATGAAGGGCACTGGTATGCGACCA
CCTCACCCAGGCATGGGCCCTCCCCAGAGTCCAATGGATCAACACAGCCA
AGGTTATATGTCACCACACCCATCTCCATTAGGAGCCCCAGAGCACGTCT
CCAGCCCTATGTCTGGAGGAGGCCCAACTCCACCTCAGATGCCACCAAGC
CAGCCGGGGGCCCTCATCCCAGGTGATCCGCAGGCCATGAGCCAGCCCAA
CAGAGGTCCCTCACCTTTCAGTCCTGTCCAGCTGCATCAGCTTCGAGCTC
AGATTTTAGCTTATAAAATGCTGGCCCGAGGCCAGCCCCTCCCCGAAACG
CTGCAGCTTGCAGTCCAGGGGAAAAGGACGTTGCCTGGCTTGCAGCAACA
ACAGCAGCAGCAACAGCAGCAGCAGCAGCAGCAGCAGCAGCAGCAGCAGC
AGCAACAGCAGCCGCAGCAGCAGCCGCCGCAACCACAGACGCAGCAACAA
CAGCAGCCGGCCCTTGTTAACTACAACAGACCATCTGGCCCGGGGCCGGA
GCTGAGCGGCCCGAGCACCCCGCAGAAGCTGCCGGTGCCCGCGCCCGGCG
GCCGGCCCTCGCCCGCGCCCCCCGCAGCCGCGCAGCCGCCCGCGGCCGCA
GTGCCCGGGCCCTCAGTGCCGCAGCCGGCCCCGGGGCAGCCCTCGCCCGT
CCTCCAGCTGCAGCAGAAGCAGAGCCGCATCAGCCCCATCCAGAAACCGC
AAGGCCTGGACCCCGTGGAAATTCTGCAAGAGCGGGAATACAGACTTCAG
GCCCGCATAGCTCATAGGATACAAGAACTGGAAAATCTGCCTGGCTCTTT
GCCACCAGATTTAAGAACCAAAGCAACCGTGGAACTAAAAGCACTTCGGT
TACTCAATTTCCAGCGTCAGCTGAGAGAGGAGGTGGTGGCCTGCATGCGC
AGGGACACGACCCTGGAGACGGCTCTCAACTCCAAAGCATACAAACGGAG
CAAGCGCCAGACTCTGAGAGAAGCTCGCATGACCGAGAAGCTGGAGAAGC
AGCAGAAGATTGAGCAGGAGAGGAAACGCCGTCAGAAACACCAGGAATAC
CTGAACAGTATTTTGCAACATGCAAAAGATTTTAAGGAATATCATCGGTC
TGTGGCCGGAAAGATCCAGAAGCTCTCCAAAGCAGTGGCAACTTGGCATG
CCAACACTGAAAGAGAGCAGAAGAAGGAGACAGAGCGGATTGAAAAGGAG
AGAATGCGGCGACTGATGGCTGAAGATGAGGAGAGTTATAGAAAACTGAT
TGATCAAAAGAAAGACAGGCGTTTAGCTTACCTTTTGCAGCAGACCGATG
AGTATGTAGCCAATCTGACCAATCTGGTTTGGGAGCACAAGCAAGCCCAG
GCAGCCAAAGAGAAGAAGAAGAGGAGGAGGAGGAAGAAGAAGGCTGAGGA
GAATGCAGAGGGTGGGGAGTCTGCCCTGGGACCGGATGGAGAGCCCATAG
ATGAGAGCAGCCAGATGAGTGACCTCCCTGTCAAAGTGACTCACACAGAA
ACCGGCAAGGTTCTGTTCGGACCAGAAGCACCCAAAGCAAGTCAGCTGGA
CGCCTGGCTGGAAATGAATCCTGGTTATGAAGTTGCCCCTAGATCTGACA
GTGAAGAGAGTGATTCTGATTATGAGGAAGAGGATGAGGAAGAAGAGTCC
AGTAGGCAGGAAACCGAAGAGAAAATACTCCTGGATCCAAATAGCGAAGA
AGTTTCTGAGAAGGATGCTAAGCAGATCATTGAGACAGCTAAGCAAGACG
TGGATGATGAATACAGCATGCAGTACAGTGCCAGGGGCTCCCAGTCCTAC
TACACCGTGGCTCATGCCATCTCGGAGAGGGTGGAGAAACAGTCTGCCCT
CCTAATTAATGGGACCCTAAAGCATTACCAGCTCCAGGGCCTGGAATGGA
TGGTTTCCCTGTATAATAACAACTTGAACGGAATCTTAGCCGATGAAATG
GGGCTTGGAAAGACCATACAGACCATTGCACTCATCACTTATCTGATGGA
GCACAAAAGACTCAATGGCCCCTATCTCATCATTGTTCCCCTTTCGACTC
TATCTAACTGGACATATGAATTTGACAAATGGGCTCCTTCTGTGGTGAAG
ATTTCTTACAAGGGTACTCCTGCCATGCGTCGCTCCCTTGTCCCCCAGCT
ACGGAGTGGCAAATTCAATGTCCTCTTGACTACTTATGAGTATATTATAA
AAGACAAGCACATTCTTGCAAAGATTCGGTGGAAATACATGATAGTGGAC
GAAGGCCACCGAATGAAGAATCACCACTGCAAGCTGACTCAGGTCTTGAA
CACTCACTATGTGGCCCCCAGAAGGATCCTCTTGACTGGGACCCCGCTGC
AGAATAAGCTCCCTGAACTCTGGGCCCTCCTCAACTTCCTCCTCCCAACA
ATTTTTAAGAGCTGCAGCACATTTGAACAATGGTTCAATGCTCCATTTGC
CATGACTGGTGAAAGGGTGGACTTAAATGAAGAAGAAACTATATTGATCA
TCAGGCGTCTACATAAGGTGTTAAGACCATTTTTACTAAGGAGACTGAAG
AAAGAAGTTGAATCCCAGCTTCCCGAAAAAGTGGAATATGTGATCAAGTG
TGACATGTCAGCTCTGCAGAAGATTCTGTATCGCCATATGCAAGCCAAGG
GGATCCTTCTCACAGATGGTTCTGAGAAAGATAAGAAGGGGAAAGGAGGT
GCTAAGACACTTATGAACACTATTATGCAGTTGAGAAAAATCTGCAACCA
CCCATATATGTTTCAGCACATTGAGGAATCCTTTGCTGAACACCTAGGCT
ATTCAAATGGGGTCATCAATGGGGCTGAACTGTATCGGGCCTCAGGGAAG
TTTGAGCTGCTTGATCGTATTCTGCCAAAATTGAGAGCGACTAATCACCG
AGTGCTGCTTTTCTGCCAGATGACATCTCTCATGACCATCATGGAGGATT
ATTTTGCTTTTCGGAACTTCCTTTACCTACGCCTTGATGGCACCACCAAG
TCTGAAGATCGTGCTGCTTTGCTGAAGAAATTCAATGAACCTGGATCCCA
GTATTTCATTTTCTTGCTGAGCACAAGAGCTGGTGGCCTGGGCTTAAATC
TTCAGGCAGCTCATACAGTGGTCATCTTTGACAGCGACTGGAATCCTCAT
CAGGATCTGCAGGCCCAAGACCGAGCTCACCGCATCGGGCAGCAGAACGA
GGTCCGGGTACTGAGGCTCTGTACCGTGAACAGCGTGGAGGAAAAGATCC
TCGCGGCCGCAAAATACAAGCTGAACGTGGATCAGAAAGTGATCCAGGCG
GGCATGTTTGACCAAAAGTCTTCAAGCCACGAGCGGAGGGCATTCCTGCA
GGCCATCTTGGAGCATGAAGAGGAAAATGAGGAAGAAGATGAAGTACCGG
ACGATGAGACTCTGAACCAAATGATTGCTCGACGAGAAGAAGAATTTGAC
CTTTTTATGCGGATGGACATGGACCGGCGGAGGGAAGATGCCCGGAACCC
GAAACGGAAGCCCCGTTTAATGGAGGAGGATGAGCTGCCCTCCTGGATCA
TTAAGGATGACGCTGAAGTAGAAAGGCTCACCTGTGAAGAAGAGGAGGAG
AAAATATTTGGGAGGGGGTCCCGCCAGCGCCGTGACGTGGACTACAGTGA
CGCCCTCACGGAGAAGCAGTGGCTAAGGGCCATCGAAGACGGCAATTTGG
AGGAAATGGAAGAGGAAGTACGGCTTAAGAAGCGAAAAAGACGAAGAAAT
GTGGATAAAGATCCTGCAAAAGAAGATGTGGAAAAAGCTAAGAAGAGAAG
AGGCCGCCCTCCCGCTGAGAAACTGTCACCAAATCCCCCCAAACTGACAA
AGCAGATGAACGCTATCATCGATACTGTGATAAACTACAAAGATAGTTCA
GGGCGACAGCTCAGTGAAGTCTTCATTCAGTTACCTTCAAGGAAAGAATT
ACCAGAATACTATGAATTAATTAGGAAGCCAGTGGATTTCAAAAAAATAA
AGGAAAGGATTCGTAATCATAAGTACCGGAGCCTAGGCGACCTGGAGAAG
GATGTCATGCTTCTCTGTCACAACGCTCAGACGTTCAACCTGGAGGGATC
CCAGATCTATGAAGACTCCATCGTCTTACAGTCAGTGTTTAAGAGTGCCC
GGCAGAAAATTGCCAAAGAGGAAGAGAGTGAGGATGAAAGCAATGAAGAG
GAGGAAGAGGAAGATGAAGAAGAGTCAGAGTCCGAGGCAAAATCAGTCAA
GGTGAAAATTAAGCTCAATAAAAAAGATGACAAAGGCCGGGACAAAGGGA
AAGGCAAGAAAAGGCCAAATCGAGGAAAAGCCAAACCTGTAGTGAGCGAT
TTTGACAGCGATGAGGAGCAGGATGAACGTGAACAGTCAGAAGGAAGTGG
GACGGATGATGAGTGATCAGTATGGACCTTTTTCCTTGGTAGAACTGAAT
TCCTTCCTCCCCTGTCTCATTTCTACCCAGTGAGTTCATTTGTCATATAG
GCACTGGGTTGTTTCTATATCATCATCGTCTATAAACTAGCTTTAGGATA
GTGCCAGACAAACATATGATATCATGGTGTAAAAAACACACACATACACA
AATATTTGTGACCAAATGGGCCTCAAAGATTCAGATTGAAACAAACAAAA
AGCTTTT
SMARCA4

SEQ ID NO:132

>gi|4507072|ref|NN_003072.1|Homo sapiens SWI/SNF
related, matrix associated, actin dependent
regulator of chromatin, subfamily a, member 4
(SMARCA4), mRNA
TTCCGGCGGGGGAGGCGCCGGGAAGTCGATGGCGCCGGCGGCTCCTGCAG
GAGGCCACTGTCTGCAGCTCCCGTGAAGATGTCCACTCCAGACCCACCCC
TGGGCGGAACTCCTCGGCCAGGTCCTTCCCCGGGCCCTGGCCCTTCCCCT
GGAGCCATGCTGGGCCCTAGCCCCGGGTCCCTCGCCGGGCTCCGCCACAG
CATGATGGGGCCCAGCCCAGGGCCGCCCTCAGCAGGACACCCCATCCCCA
CCCAGGGGCCTGGAGGGTACCCTCAGGACAACATGCACCAGATGCACAAG
CCCATGGAGTCCATGCATGAGAAGGGCATGTCGGACGACCCGCGCTACAA
CCAGATGAAAGGAATGGGGATGCGGTCAGGGGGCCATGCTGGGATGGGGC
CCCCGCCCAGCCCCATGGACCAGCACTCCCAAGGTTACCCCTCGCCCCTG
GGTGGCTCTGAGCATGCCTCTAGTCCAGTTCCAGCCAGTGGCCCGTCTTC
GGGGCCCCAGATGTCTTCCGGGCCAGGAGGTGCCCCGCTGGATGGTGCTG
ACCCCCAGGCCTTGGGGCAGCAGAACCGGGGCCCAACCCCATTTAACCAG
AACCAGCTGCACCAGCTCAGAGCTCAGATCATGGCCTACAAGATGCTGGC
CAGGGGGCAGCCCCTCCCCGACCACCTGCAGATGGCGGTGCAGGGCAAGC
GGCCGATGCCCGGGATGCAGCAGCAGATGCCAACGCTACCTCCACCCTCG
GTGTCCGCAACAGGACCCGGCCCTGGCCCTGGCCCTGGCCCCGGCCCGGG
TCCCGGCCCGGCACCTCCAAATTACAGCAGGCCTCATGGTATGGGAGGGC
CCAACATGCCTCCCCCAGGACCCTCGGGCGTGCCCCCCGGGATGCCAGGC
CAGCCTCCTGGAGGGCCTCCCAAGCCCTGGCCTGAAGGACCCATGGCGAA
TGCTGCTGCCCCCACGAGCACCCCTCAGAAGCTGATTCCCCCGCAGCCAA
CGGGCCGCCCTTCCCCCGCGCCCCCTGCCGTCCCACCCGCCGCCTCGCCC
GTGATGCCACCGCAGACCCAGTCCCCCGGGCAGCCGGCCCAGCCCGCGCC
CATGGTGCCACTGCACCAGAAGCAGAGCCGCATCACCCCCATCCAGAAGC
CGCGGGGCCTCGACCCTGTGGAGATCCTGCAGGAGCGCGAGTACAGGCTG
CAGGCTCGCATCGCACACCGAATTCAGGAACTTGAAAACCTTCCCGGGTC
CCTGGCCGGGGATTTGCGAACCAAAGCGACCATTGAGCTCAAGGCCCTCA
GGCTGCTGAACTTCCAGAGGCAGCTGCGCCAGGAGGTGGTGGTGTGCATG
CGGAGGGACACAGCGCTGGAGACAGCCCTCAATGCTAAGGCCTACAAGCG
CAGCAAGCGCCAGTCCCTGCGCGAGGCCCGCATCACTGAGAAGCTGGAGA
AGCAGCAGAAGATCGAGCAGGAGCGCAAGCGCCGGCAGAAGCACCAGGAA
TACCTCAATAGCATTCTCCAGCATGCCAAGGATTTCAAGGAATATCACAG
ATCCGTCACAGGCAAAATCCAGAAGCTGACCAAGGCAGTGGCCACGTACC
ATGCCAACACGGAGCGGGAGCAGAAGAAAGAGAACGAGCGGATCGAGAAG
GAGCGCATGCGGAGGCTCATGGCTGAAGATGAGGAGGGGTACCGCAAGCT
CATCGACCAGAAGAAGGACAAGCGCCTGGCCTACCTCTTGCAGCAGACAG
ACGAGTACGTGGCTAACCTCACGGAGCTGGTGCCGCAGCACAAGGCTGCC
CAGGTCGCCAAGGAGAAAAAGAAGAAAAAGAAAAAGAAGAAGGCAGAAAA
TGCAGAAGGACAGACGCCTGCCATTGGGCCGGATGGCGAGCCTCTAGACG
AGACCAGCCAGATGAGCGACCTCCCGGTGAAGGTGATCCACGTGGAGAGT
GGGAAGATCCTCACAGGCACAGATGCCCCCAAAGCCGGGCAGCTGGAGGC
CTGGCTCGAGATGAACCCGGGGTATGAAGTAGCTCCGAGGTCTGATAGTG
AAGAAAGTGGCTCAGAAGAAGAGGAAGAGGAGGAGGAGGAAGAGCAGCCG
CAGGCAGCACAGCCTCCCACCCTGCCCGTGGAGGAGAAGAAGAAGATTCC
AGATCCAGACAGCGATGACGTCTCTGAGGTGGACGCGCGGCACATCATTG
AGAATGCCAAGCAAGATGTCGATGATGAATATGGCGTGTCCCAGGCCCTT
GCACGTGGCCTGCAGTCCTACTATGCCGTGGCCCATGCTGTCACTGAGAG
AGTGGACAAGCAGTCAGCGCTTATGGTCAATGGTGTCCTCAAACAGTACC
AGATCAAAGGTTTGGAGTGGCTGGTGTCCCTGTACAACAACAACCTGAAC
GGCATCCTGGCCGACGAGATGGGCCTGGGGAAGACCATCCAGACCATCGC
GCTCATCACGTACCTCATGGAGCACAAACGCATCAATGGGCCCTTCCTCA
TCATCGTGCCTCTCTCAACGCTGTCCAACTGGGCGTACGAGTTTGACAAG
TGGGCCCCCTCCGTGGTGAAGGTGTCTTACAAGGGATCCCCAGCAGCAAG
ACGGGCCTTTGTCCCCCAGCTCCGGAGTGGGAAGTTCAACGTCTTGCTGA
CGACGTACGAGTACATCATCAAAGACAAGCACATCCTCGCCAAGATCCGT
TGGAAGTACATGATTGTGGACGAAGGTCACCGCATGAAGAACCACCACTG
CAAGCTGACGCAGGTGCTCAACACGCACTATGTGGCACCCCGCCGCCTGC
TGCTGACGGGCACACCGCTGCAGAACAAGCTTCCCGAGCTCTGGGCGCTG
CTCAACTTCCTGCTGCCCACCATCTTCAAGAGCTGCAGCACCTTCGAGCA
GTGGTTTAACGCACCCTTTGCCATGACCGGGGAAAAGGTGGACCTGAATG
AGGAGGAAACCATTCTCATCATCCGGCGTCTCCACAAAGTGCTGCGGCCC
TTCTTGCTCCGACGACTCAAGAAGGAAGTCGAGGCCCAGTTGCCCGAAAA
GGTGGAGTACGTCATCAAGTGCGACATGTCTGCGCTGCAGCGAGTGCTCT
ACCGCCACATGCAGGCCAAGGGCGTGCTGCTGACTGATGGCTCCGAGAAG
GACAAGAAGGGCAAAGGCGGCACCAAGACCCTGATGAACACCATCATGCA
GCTGCGGAAGATCTGCAACCACCCCTACATGTTCCAGCACATCGAGGAGT
CCTTTTCCGAGCACTTGGGGTTCACTGGCGGCATTGTCCAAGGGCTGGAC
CTGTACCGAGCCTCGGGTAAATTTGAGCTTCTTGATAGAATTCTTCCCAA
ACTCCGAGCAACCAACCACAAAGTGCTGCTGTTCTGCCAAATGACCTCCC
TCATGACCATCATGGAAGATTACTTTGCGTATCGCGGCTTTAAATACCTC
AGGCTTGATGGAACCACGAAGGCGGAGGACCGGGGCATGCTGCTGAAAAC
CTTCAACGAGCCCGGCTCTGAGTACTTCATCTTCCTGCTCAGCACCCGGG
CTGGGGGGCTCGGCCTGAACCTCCAGTCGGCAGACACTGTGATCATTTTT
GACAGCGACTGGAATCCTCACCAGGACCTGCAAGCGCAGGACCGAGCCCA
CCGCATCGGGCAGCAGAACGAGGTGCGTGTGCTCCGCCTCTGCACCGTCA
ACAGCGTGGAGGAGAAGATCCTAGCTGCAGCCAAGTACAAGCTCAACGTG
GACCAGAAGGTGATCCAGGCCGGCATGTTCGACCAGAAGTCCTCCAGCCA
TGAGCGGCGCGCCTTCCTGCAGGCCATCCTGGAGCACGAGGAGCAGGATG
AGAGCAGACACTGCAGCACGGGCAGCGGCAGTGCCAGCTTCGCCCACACT
GCCCCTCCGCCAGCGGGCGTCAACCCCGACTTGGAGGAGCCACCTCTAAA
GGAGGAAGACGAGGTGCCCGACGACGAGACCGTCAACCAGATGATCGCCC
GGCACGAGGAGGAGTTTGATCTGTTCATGCGCATGGACCTGGACCGCAGG
CGCGAGGAGGCCCGCAACCCCAAGCGGAAGCCGCGCCTCATGGAGGAGGA
CGAGCTCCCCTCGTGGATCATCAAGGACGACGCGGAGGTGGAGCGGCTGA
CCTGTGAGGAGGAGGAGGAGAAGATGTTCGGCCGTGGCTCCCGCCACCGC
SMARCA3

SEQ ID NO:133

>gi|4507070|ref|NM_003071.1|Homo sapiens SWI/SNF
related, matrix associated, actin dependent
regulator of chromatin, subfamily a, member 3
(SMARCA3), cnRNA
ATTCCCGGGGTCTGACTGGACTCGCGGCGACTTACCTTTCAGTCGTGCGC
TCCTGATCCGGCGCTCGGAATTTGTCCCCGGCTTCAGGGCTGCGGGGCCT
GGAAGGAGGCGTATCGAGGCGGCTCGAAAACGATCCAGGGGAGCCGAGGC
GCTCCTCTTGTCATCCCACTCAGCGCCATGTCCTGGATGTTCAAGAGGGA
TCCAGTTTGGAAGTACTTGCAGACTGTCCAGTATGGAGTTCATGGAAATT
TTCCACGCCTCTCATATCCAACTTTCTTTCCACGTTTTGAATTCCAAGAT
GTTATCCCTCCAGATGACTTTCTAACTAGTGATGAAGAAGTAGATTCCGT
TTTATTTGGAAGTTTGAGAGGTCATGTGGTTGGACTACGCTATTACACGG
GAGTAGTTAATAATAATGAAATGGTTGCATTACAACGAGATCCTAATAAC
CCTTATGATAAGAATGCAATTAAAGTAAACAATGTGAATGGAAATCAAGT
TGGCCATTTAAAGAAAGAGCTTGCAGGTGCTTTGGCCTATATCATGGACA
ACAAATTGGCACAAATTGAAGGGGTAGTTCCTTTTGGTGCAAACAATGCT
TTTACCATGCCTCTGCATATGACTTTTTGGGGAAAAGAAGAAAATAGAAA
AGCGGTTTCAGATCAGTTGAAGAAACATGGATTTAAATTGGGTCCTGCAC
CAAAAACTTTAGGATTCAATTTGGAAAGTGGTTGGGGCTCTGGAAGAGCT
GGACCAAGCTATAGTATGCCAGTGCATGCTGCAGTACAGATGACAACTGA
ACAGCTTAAAACAGAATTTGACAAATTGTTTGAAGATTTAAAAGAAGATG
ATAAAACCCATGAAATGGAACCAGCTGAGGCTATTGAAACACCACTGCTT
CCACATCAAAAACAAGCTCTAGCTTGGATGGTGTCACGGGAAAATAGCAA
AGAACTTCCACCATTCTGGGAACAGCGAAATGACTTATACTATAACACAA
TAACAAATTTTTCTGAGAAGGACCGACCAGAAAATGTCCATGGAGGAATT
TTAGCTGATGATATGGGTTTGGGTAAAACTCTTACAGCCATTGCAGTAAT
CCTTACCAACTTCCATGATGGCAGACCTCTTCCTATTGAAAGAGTTAAAA
AGAATCTACTGAAGAAGGAATATAATGTTAACGATGACTCTATGAAACTT
GGAGGAAACAATACCAGTGAAAAGGCAGATGGACTAAGCAAAGACGCATC
TAGATGTAGTGAACAACCCAGTATTTCAGATATCAAGGAGAAGAGTAAGT
TTCGCATGTCAGAATTGTCTACGTCCCGCCCCAAAAGAAGAAAAACTGCT
GTCCAGTACATAGAAAGCAGTGATTCAGAGGAAATTGAAACAAGTGAATT
GCCGCAGAAAATGAAAGGCAAACTGAAAAATGTACAGTCTGAAACTAAAG
GCAGGGCGAAAGCAGGATCTTCTAAGGTTATAGAAGATGTGGCATTTGCA
TGTGCATTAACTTCATCTGTTCCTACAACAAAAAAGAAAATGTTGAAAAA
GGGAGCTTGTGCAGTGGAGGGGTCAAAGAAAACTGATGTTGAGGAGAGAC
CAAGAACAACACTGATCATCTGTCCGCTTTCTGTGTTAAGCAACTGGATT
GACCAGTTTGGACAACATATAAAATCAGATGTACACTTGAATTTTTATGT
TTATTATGGTCCTGATCGTATTAGAGAACCGGCCTTACTTTCAAAACAGG
ATATTGTTTTGACTACGTATAATATTTTAACTCATGACTATGGAACTAAA
GGAGATAGTCCATTACATAGCATAAGGTGGCTAAGAGTGATCCTGGATGA
AGGACATGCCATACGAAATCCAAATGCTCAGCAGACAAAAGCTGTACTTG
ACTTAGAATCAGAAAGAAGATGGGTTTTGACAGGTACTCCAATCCAGAAT
TCTTTAAAGGACTTGTGGTCTCTTCTTTCCTTTTTAAAACTTAAACCATT
TATTGATAGAGAATGGTGGCATAGAACAATACAGCGTCCTGTCACAATGG
GAGATGAAGGAGGACTTAGGCGTTTACAGTCCCTAATTAAAAATATTACA
CTTAGAAGAACAAAGACAAGCAAAATTAAAGGAAAACCTGTTTTGGAGTT
ACCAGAACGTAAAGTATTTATTCAGCACATTACACTTTCAGATGAAGAGA
GAAAGATTTATCAGTCTGTGAAAAATGAAGGCAGAGCCACTATTGGAAGG
TATTTTAATGAAGGGACTGTCCTGGCACATTATGCAGATGTCCTGGGTCT
TTTGCTTAGACTGCGGCAAATTTGTTGCCATACTTACCTTCTTACAAATG
CAGTGTCTTCCAATGGCCCCTCAGGAAATGATACACCTGAAGAACTGAGA
AAGAAGTTAATAAGGAAGATGAAGTTAATTCTGAGCTCAGGTTCAGATGA
GGAATGTGCAATTTGCCTGGATTCTTTAACAGTTCCTGTGATAACACATT
GTGCACATGTATTTTGTAAACCCTGTATTTGCCAAGTCATTCAGAATGAG
CAGCCACATGCTAAATGCCCTTTATGCAGAAATGATATACATGAAGATAA
TTTATTAGAATGTCCTCCAGAAGAATTAGCACGTGACAGTGAGAAAAAGT
CTGATATGGAATGGACATCCAGTTCAAAGATTAATGCGCTAATGCACGCA
TTGACTGACTTAAGAAAGAAGAATCCCAACATAAAAAGTTTGGTTGTTTC
TCAGTTTACAACATTCCTGTCTTTAATAGAAATACCACTTAAAGCCTCTG
GATTTGTGTTTACTCGTTTGGATGGTTCCATGGCCCAAAAGAAAAGAGTT
GAATCAATTCAGTGTTTTCAAAACACTGAAGCAGGATCTCCAACTATAAT
GCTTCTGTCCTTAAAAGCAGGTGGAGTTGGTTTGAATCTGTCTGCAGCTT
CTCGAGTGTTTTTAATGGATCCAGCCTGGAATCCTGCTGCTGAAGATCAG
TGCTTTGACAGATGCCATAGACTTGGTCAGAAGCAAGAAGTTATCATCAC
AAAATTCATTGTAAAGGACTCTGTTGAAGAAAATATGCTGAAAATACAAA
ACAAAAAGAGAGAACTTGCAGCAGGAGCCTTTGGAACTAAAAAACCAAAT
GCTGACGAAATGAAACAAGCCAAAATTAATGAAATCAGAACATTAATTGA
CTTATAATTTGTGGGATTTTAGTAAGAAGACTACTATATGTGAGAGGCGT
GATATCTGGATGGAAGTTGGGCTGGATGATCTCCAAAGTCGTTTCAACTC
TTAAAGACATCTTAATCCTGAATGTAAACAATTGTTATGTGTTTAGAATC
AGAATTTGATTTTGAACTTGAGTAATTCATCCTTACAGCTATCTGTAGAA
TTAGTCATCTTTTTTCTT
SMARCAL1

SEQ ID NO:134

>gi|7657149|ref|NM_014140.1|Homo sapiens SWI/SNF
related, matrix associated, actin dependent
regulator of chromatin, subfamily a-like 1
(SMARCAL1), mRNA
GCTGGGTTGGAAAAAGACTATGTTAGCAAGTGTCACGCCATGCTTTTGCC
AACTTTCCAATTAAAGGTTGACATTCCTGCATAAACATTTCTCTGTGAAA
ATGTCCTTGCCTCTTACAGAGGAGCAGAGGAAAAAGATTGAAGAGAATCG
ACAAAAGGCTCTGGCCCGCAGAGCTGAGAAGTTATTGGCAGAACAGCATC
AGAGGACTAGCTCGGGCACCTCCATTGCTGGCAACCCATTCCAGGCCAAG
CAAGGCCCATCCCAAAATTTCCCAAGGGAGTCTTGTAAGCCAGTGAGCCA
TGGTGTCATTTTCAAGCAACAGAATCTCAGTAGCTCATCTAATGCTGACC
AAAGACCTCATGATTCCCACAGTTTTCAGGCAAAGGGAATATGGAAAAAG
CCAGAAGAAATGCCCACAGCCTGCCCAGGCCACAGTCCACGTAGTCAAAT
GACTCTCACTGGAATCTCTCCTCCCTTGGCACAAAGTCCTCCAGAGGTCC
CTAAACAACAGCTCTTGAGTTATGAGTTAGGTCAAGGTCATGCTCAGGCT
TCACCTGAGATCAGGTTCACACCCTTTGCTAACCCAACTCATAAGCCTCT
GGCCAAACCAAAGAGTTCCCAAGAGACACCAGCTCATTCCTCTGGACAGC
CTCCCAGGGATGCTAAGTTAGAGGCCAAGACAGCAAAAGCCTCCCCTTCG
GGGCAGAACATTTCTTACATCCATTCTAGCTCAGAGAGTGTAACGCCCAG
GACAGAAGGAAGACTCCAGCAGAAGTCAGGGTCCTCAGTCCAAAAAGGAG
TGAACTCTCAGAAGGGAAAGTGCGTAAGGAACGGCGATCGTTTCCAGGTG
TTGATTGGGTACAATGCGGAACTCATTGCAGTGTTTAAGACCCTGCCCAG
CAAGAATTATGATCCTGACACCAAGACGTGGAACTTCAGCATGAATGACT
ATAGTGCCCTGATGAAAGCAGCCCAGAGCCTCCCCACGGTCAACCTGCAG
CCTCTGGAATGGGCCTATGGGAGCAGCGAGTCACCCTCCACCAGCAGTGA
GGGACAGGCCGGCCTTCCATCAGCTCCATCCCTTTCATTTGTCAAAGGGC
GAGGCATGCTCATCTCCAGGGCCTACTTCGAGGCAGACATCAGTTATTCA
CAGGACCTTATTGCGCTTTTTAAACAGATGGATTCCAGAAGATATGATGT
CAAGACCAGGAAGTGGAGCTTTCTCTTGGAAGAGCACAGTAAACTAATTG
CAAAGGTGCGCTGCCTCCCACAAGTTCAGCTGGACCCTCTGCCCACGACT
CTCACCCTGGCGTTTGCTTCTCAGCTCAAGAAGACATCTCTCAGTCTCAC
GCCAGATGTCCCAGAGGCAGACCTTTCTGAAGTGGACCCCAAGCTCGTGT
CTAATCTGATGCCCTTTCAGAGAGCTGGAGTCAATTTTGCCATAGCCAAA
GGAGGCCGCCTGCTGCTCGCTGACGACATGGGCCTGGGGAAGACCATCCA
AGCCATCTGCATCGCAGCCTTTTACCGGAAGGAGTGGCCGCTCCTGGTGG
TGGTGCCATCCTCCGTGCGCTTCACCTGGGAGCAGGCCTTCCTTCGGTGG
CTGCCATCTCTGAGCCCAGATTGCATCAACGTCGTGGTGACTGGGAAGGA
CCGCCTGACAGCTGGCCTGATCAACATTGTCAGCTTTGACCTTCTTAGCA
AGTTGGAAAAACAGCTAAAAACCCCTTTTAAAGTTGTCATCATTGATGAA
TCTCACTTCCTCAAAAACAGTAGGACTGCCCGCTGTCGAGCAGCTATGCC
GGTCCTAAAGGTCGCCAAGAGGGTGATCCTGTTGTCGGGCACACCAGCCA
TGTCCCGGCCCGCAGAGCTCTACACGCAGATCATCGCAGTCAAGCCAACT
TTCTTCCCCCAGTTTCATGCCTTTGGACTTCGCTACTGTGATGCCAAACG
GATGCCTTGGGGGTGGGACTACTCAGGTTCCTCCAACCTGGGAGAGCTGA
AGCTCCTGCTGGAGGAAGCAGTCATGCTGCGGCGCCTCAAGTCCGACGTC
CTTTCCCAGCTGCCTGCCAAGCAGCGCAAGATAGTGGTGATTGCCCCAGG
ACGGATCAATGCCAGGACCAGAGCTGCCCTGGATGCTGCAGCCAAGGAAA
TGACCACCAAGGACAAAACTAAACAGCAGCAGAAAGATGCCCTCATTCTC
TTCTTCAACAGAACAGCTGAAGCTAAAATCCCATCTGTCATTGAATATAT
CTTGGACCTACTGGAAAGTGGAAGAGAGAAGTTTTTAGTATTTGCACACC
ATAAGGTGGTCCTGGACGCAATTACGCAAGAGCTTGAGAGAAAGCACGTG
CAGCACATCCGCATCGATGGCTCCACCTCATCAGCTGAGCGGGAGGACCT
GTGCCAGCAGTTCCAACTGTCGGAGAGGCATGCTGTGGCCGTGCTGTCCA
TCACCGCTGCCAATATGGGCCTCACCTTCTCCTCGGCTGACCTGGTGGTG
TTTGCTGAGCTGTTTTGGAACCCAGGGGTGCTGATCCAGGCTGAGGACCG
CGTGCACCGCATTGGACAGACCAGCTCCGTGGGCATTCACTACCTCGTGG
CAAAGGGCACAGCTGATGACTACCTTTGGCCCCTGATTCAAGAGAAGATT
AAAGTTCTGGCAGAAGCCGGGCTTTCTGAGACCAATTTTTCAGAAATGAC
AGAATCCACTGATTACCTCTACAAGGACCCAAAGCAGCAGAAGATGTACG
ACCTATTCCAGAAGTCCTTTGAGAAAGAAGGAAGTGATATGGAGCTCGTG
GAAGCAGCAGAGTCCTTTGACCCAGGAAGTGGTTCAGGAACATCTGGAAG
TAGTTCCCAGAACATGGGAGACACCCTGGATGAAAGCTCATTGACAGCCA
GTCCACAGAAGAAAAGGAGATTTGAATTTTTTGATAACTGGGACAGCTTT
ACGTCTCCCCTGTAAAAGGGGCAAAAAAAAAAAAAAAAAAAGCATTTTAA
AATCAAAAAGAAAAAAATAAAA
SMARCA1

SEQ ID NO:135

>gi|450766|ref|NM_003069.1|Homo sapiens SWI/SNF
related, matrix associated, actin dependent
regulator of chromatin, subfamily a, member 1
(SMARCA1), mRNA
CAAGACTGGAAGCAGAGAGAGAGAGCAAGAGTGAGAGAGAGCGAGCGAGC
GTAGTCAGGAGATGGTGTTGTATTCCAGGAGAAAAACGTTTCTTCATTTC
AACTCAAACTTGCTGCTAAAGCGCCTAAATCTGAAAAGGAAATGGACCCA
GAATATGAAGAGAAAATGAAAGCCGACCGAGCAAAGAGATTTGAATTTTT
ACTGAAGCAGACAGAACTTTTTGCACATTTCATTCAGCCTTCAGCACAGA
AATCTCCAACATCTCCACTGAACATGAAATTGGGACGTCCCCGAATAAAG
AAAGATGAAAAGCAGAGCTTAATTTCTGCTGGAGACTACCGCCATAGGCG
CACAGAGCAAGAAGAAGATGAAGAGCTACTGTCTGAGAGTCGGAAAACAT
CTAATGTGTGTATTAGATTTGAGGTGTCACCTTCATATGTGAAAGGGGGG
CCACTGAGAGATTATCAGATTCGAGGACTGAATTGGTTGATCTCTTTATA
TGAAAATGGAGTCAATGGCATTTTGGCTGATGAAATGGGCCTTGGGAAAA
CTTTACAAACAATTGCTTTGCTTGGTTACCTGAAACACTACCGAAATATT
CCTGGACCTCACATGGTTTTAGTTCCAAAGTCTACTTTACACAACTGGAT
GAATGAATTTAAACGATGGGTCCCATCTCTCCGTGTCATTTGTTTTGTCG
GAGACAAGGATGCCAGAGCTGCTTTTATTCGTGATGAAATGATGCCAGGA
GAGTGGGATGTTTGCGTTACTTCTTATGAGATGGTAATTAAAGAAAAATC
TGTATTCAAAAAGTTTCACTGGCGATACCTGGTCATTGATGAAGCTCACA
GAATAAAGAATGAAAAATCTAAGCTTTCAGAGATTGTTCGTGAGTTCAAG
TCGACTAACCGCTTGCTCCTAACTGGAACACCTTTGCAGAATAACCTGCA
TGAACTGTGGGCCTTACTCAACTTTTTATTGCCTGATGTCTTTAATTCTG
CAGATGACTTTGATTCTTGGTTTGACACTAAAAATTGTCTTGGTGATCAA
AAACTCGTGGAAAGACTTCATGCAGTTTTAAAACCATTTTTGTTACGCCG
TATAAAAACTGATGTAGAGAAGAGTCTGCCACCTAAAAAGGAAATAAAGA
TTTACTTGGGGCTGAGTAAGATGCAACGAGAATGGTATACAAAAATCCTG
ATGAAAGATATTGATGTTTTAAACTCTTCTGGCAAGATGGACAAGATGCG
ACTCTTAAACATTCTGATGCAGCTTCGAAAGTGTTGTAATCATCCATATC
TGTTTGATGGTGCTGAACCTGGTCCACCTTATACCACTGATGAGCATATT
GTCAGCAACAGTGGTAAAATGGTAGTTCTGGATAAACTATTGGCCAAACT
CAAAGAACAGGGTTCAAGGGTTCTCATTTTCAGCCAGATGACTCGCTTGC
TGGATATTTTGGAAGATTATTGCATGTGGCGTGGTTATGAGTATTGTCGA
CTGGATGGACAAACCCCGCATGAAGAAAGAGAGGATAAATTCCTAGAAGT
GGAATTTCTGGGTCAAAGGGAAGCAATAGAGGCTTTTAATGCTCCTAATA
GTAGCAAATTCATCTTTATGCTAAGTACCAGGGCTGGAGGTCTCGGAATT
AACCTGGCAAGTGCTGATGTGGTTATACTATATGATTCAGACTGGAACCC
ACAGGTTGATCTACAAGCTATGGATCGAGCACATCGTATTGGTCAGAAGA
AACCAGTACGTGTATTCCGTCTCATCACTGACAACACTGTTGAAGAGAGG
ATTGTAGAAAGAGCTGAGATAAAACTGAGACTCGATTCAATTGTTATACA
ACAAGGAAGACTCATTGACCAACGGTCTAACAAGCTGGCAAAAGAGGAAA
TGTTACAAATGATACGGCATGGAGCCACCCATGTTTTTGCTTCTAAAGAG
AGTGAGTTGACAGATGAAGACATTACAACTATTCTGGAAAGAGGGGAAAA
GAAGACTGCAGAGATGAATGAACGCCTGCAAAAAATGGGAGAGTCTTCTC
TAAGAAATTTTAGAATGGACATTGAACAAAGTTTATACAAATTTGAGGGA
GAAGATTATAGAGAAAAACAGAAGCTTGGCATGGTGGAATGGATTGAACC
TCCTAAACGAGAACGCAAAGCAAACTACGCAGTGGATGCCTACTTTAGAG
AGGCTTTGCGTGTCAGCGAGCCAAAGATTCCAAAGGCTCCACGGCCTCCA
AAACAGCCAAATGTTCAGGATTTTCAATTTTTCCCACCACGCTTATTTGA
GCTCCTGGAAAAGGAAATTCTTTATTATCGGAAGACAATAGGCTATAAGG
TTCCAAGGAATCCTGATATCCCAAATCCAGCTCTGGCTCAAAGAGAAGAG
CAAAAAAAGATTGATGGAGCTGAACCTCTTACACCAGAAGAGACTGAAGA
AAAGGAAAAACTTCTCACACAAGGTTTCACAAACTGGACTAAACGAGATT
TTAACCAGTTTATTAAAGCTAATGAGAAATATGGAAGAGATGACATTGAT
AACATAGCTCGAGAGGTAGAGGGCAAATCCCCTGAGGAGGTCATGGAGTA
TTCAGCTGTATTTTGGGAACGTTGCAATGAATTACAGGACATTGAGAAAA
TTATGGCTCAAATTGAACGTGGAGAAGCAAGAATTCAACGAAGGATCAGT
ATCAAGAAAGCCCTGGATGCCAAAATTGCAAGATACAAGGCTCCATTTCA
TCAGTTGCGCATTCAGTATGGAACCAGCAAAGGAAAGAACTATACTGAGG
AAGAAGATAGATTCTTGATTTGTATGTTACACAAAATGGGCTTTGATAGA
GAAAATGTATATGAAGAATTAAGACAGTGTGTACGAAATGCTCCCCAGTT
TAGATTTGACTGGTTTATCAAGTCTAGGACTGCCATGGAATTCCAGAGAC
GCTGTAACACTCTGATTTCATTGATTGAGAAAGAAAATATGGAAATTGAG
GAAAGAGAGAGAGCAGAAAAGAAGAAACGGGCAACTAAAACTCCAATGGT
AAAATTTTCAGCATTTTCCTAACTTTTAGATTTAACATTGTTGGGCCATT
TAAAATGTGCATATTGGAGCAGAACATTAAATCTGTTTCCATTTTAGTCA
CAGAAAAGAAAAGCAGAGTCAGCTACTGAGAGCTCTGGAAAGAAGGATGT
CAAGAAGGTGAAATCCTAAAGCCTAGAAATAAAGTTTTAAATGGGAAACT
GCTATTTTCTTGTTCCCATCTTCAAATGCTAATTGCCAGTTCCAGTGTAT
TCATGGTACTCTAAGAAAAATCTCTTTGGTTTTGATTTCTTGCATATTTT
ATATATTTTACAATGCTTTCTACCTGAAATGTGTAGCTTTATATTTTATG
GCATTCTAGTATTTTTGTGTACTGTATTTTGTGCATTTCATGTCTTCATC
AAAATCCTCTCAGTCCTTGTTCTTTTGAAGCTTGTGCTGAGGTTTTAGCT
TTTCTATGTTTTATATGCCGCTGCTTTGAAAGAGAACCTAGATTCTATAG
TTGTATTATTGTTGTTTCATACTTTAAATTTATATGGCTGTGGAAAAACG
AATTAAAATGTTTTGAGGAGAAAGAAAAAAAAAA
CHRAC1

SEQ ID NO:136

>gi|8393115|ref|NM_017444.1|Homo sapiens chromatin
accessibility complex 1 (CHRAC1), mRNA
ATGGCGGACGTGGTCGTGGGTAAAGACAAGGGCGGGGAGCAGCGGCTCAT
CTCGCTGCCTCTATCCCGCATCCGGGTCATCATGAAGAGCTCCCCCGAGG
TGTCCAGCATCAACCAGGAGGCGTTGGTGCTCACGGCCAAGGCCACGGAG
CTCTTTGTTCAATGCCTAGCCACCTATTCCTACAGACACGGCAGTGGAAA
GGAAAAGAAAGTACTGACTTACAGTGATTTAGCAAACACTGCACAGCAAT
CAGAAACTTTTCAGTTTCTTGCAGATATATTACCAAAGAAGATTTTAGCT
AGTAAATACCTGAAAATGCTTAAAGAGGAAAAGAGGGAAGAAGATGAGGA
GAATGACAATGATAATGAAAGTGACCATGATGAAGCTGACTCCTAA

1-21. (canceled)

22. An inhibitory RNA (RNAi) molecule derived from a nucleic acid molecule comprising a nucleic acid sequence selected from the group consisting of:
a) nucleic acid sequences represented by any of SEQ ID NOs:7-23 or fragments thereof which encode a Notch signaling target;
b) nucleic acid sequences which hybridize to any of the nucleic acid sequences of SEQ ID NOs:7-23 and which encode a Notch signaling target gene; and
c) nucleic acid sequences which comprise sequences which are degenerate as a result of the genetic code to the nucleic acid sequences defined in a) and b).

23. An RNAi molecule according to claim 22, wherein said molecule comprises a first part linked to a second part, wherein said first and second parts are complementary over at least part of their length, and further wherein said first and second parts form a double stranded region by complementary base pairing over at least part of their length.

24. An RNAi molecule according to claim 23, wherein said first and second parts are linked by at least one nucleotide base.

25. An RNAi molecule according to claim 24, wherein said first and second parts are linked by 2, 3, 4, 5, 6, 7, 8, 9 or 10 nucleotide bases.

26. An RNAi molecule according to claim 24, wherein the linker is at least 10 nucleotide bases.

27. An RNAi molecule according to claim 22, wherein the length of said RNAi molecule is between 10 and 1000 nucleotide bases (nb).

28. An RNAi molecule according to claim 27, wherein the length of the RNA molecule is selected from 10 nb, 20 nb, 30 nb, 40 nb, 50 nb, 60 nb, 70 nb, 80 nb and 90 nb.

29. An RNAi molecule according to claim 27, wherein the RNA is 21 nb in length.

30. An RNAi molecule according to claim 27, wherein the RNA molecule is 100 nb, 200 nb, 300 nb, 400 nb, 500 nb, 600 nb, 700 nb, 800 nb, 900 nb or 1000 nb in length.

31. An RNAi molecule according to claim 22, wherein the RNA molecule is at least 1000 nb.

32. An RNAi molecule according to claim 22, wherein the RNAi molecule comprises modified nucleotide bases.

33. A nucleic acid molecule encoding at least part of a gene which modulates stem cell differentiation comprising a nucleic acid sequence selected from the group consisting of:
a) nucleic acid sequences represented by SEQ ID NOs:7-23 or fragments thereof which encode a Notch signaling target;
b) nucleic acid sequences which hybridize to any of the nucleic acid sequences of SEQ ID NOs:7-23 and which are Notch signaling target genes; and
c) nucleic acid sequences which comprise sequences which are degenerate as a result of the genetic code to any of the nucleic acid sequences defined in (a) and (b);
wherein said nucleic acid molecule comprises a first part linked to a second part, said first and second parts being complementary over at least part of their length, said nucleic acid molecule being operably linked to at least one further nucleic acid molecule capable of promoting transcription of said nucleic acid linked thereto, and said first and second parts forming a double stranded region by complementary base pairing over at least part of their length when said nucleic acid molecule is transcribed.

34. An expression vector including an expression cassette comprising at least one nucleic acid molecule according to claim 33.
